(19) United States
(12) Patent Application Publication
(10) Pub. No.: US 2024/0376076 A1
(43) Pub. Date: Nov. 14, 2024

(54) INHIBITORS TARGETING UBIQUITIN SPECIFIC PROTEASE 7 (USP7)

(71) Applicant: Dana-Farber Cancer Institute, Inc., Boston, MA (US)

(72) Inventors: Sara Buhrlage, Somerville, MA (US); Xiaoxi Liu, Burlington, MA (US); Ryan C. Schoenfeld, New York, NY (US)

(21) Appl. No.: 18/580,725

(22) PCT Filed: Jul. 20, 2022
(86) PCT No.: PCT/US2022/037756

§ 371 Date: Jan. 19, 2024

Related U.S. Application Data
(60) Provisional application No. 63/223,788, filed on Jul. 20, 2021.

(51) Int. Cl. C07D 401/14; A61K 31/506; A61K 31/517; A61K 31/519; A61K 31/53; A61K 31/5377; C07D 401/06; C07D 413/14; C07D 471/04; C07D 487/04; C07D 491/052; C07D 495/04; C07D 513/04

(52) U.S. Cl. CPC C07D 401/14; A61K 31/506; A61K 31/517; A61K 31/519; A61K 31/53; A61K 31/5377; C07D 401/06; C07D 413/14; C07D 471/04; C07D 487/04; C07D 491/052; C07D 495/04; C07D 513/04

(57) ABSTRACT

Disclosed herein are inhibitors of deubiquitinating (DUB) enzyme USP7 (Ubiquitin Specific Protease 7). Also provided are methods of treating a disease or disorder modulated by USP7.

### CROSS-REFERENCE TO RELATED APPLICATIONS

This application claims the benefit of priority to U.S. Provisional Application No. 63/223,788, filed Jul. 20, 2021, the entire contents of which are incorporated herein by reference.

### BACKGROUND

Deubiquitinating enzymes (DUBs) have garnered significant attention as drug targets in the last 5-10 years. DUB inhibitors effectively promote degradation of oncogenic proteins, especially proteins that are challenging to directly target because they are stabilized by DUB family members. Highly-optimized and well-characterized DUB inhibitors have thus become highly sought after tools. Most reported DUB inhibitors, however, are polypharmacological agents possessing weak (micromolar) potency toward their primary target, thereby limiting their utility in target validation and mechanism studies.

The DUB enzyme USP7 (Ubiquitin Specific Protease 7) has been shown to be involved in regulation of a myriad of cellular processes, including epigenetics, cell cycle, DNA repair, immunity, viral infection and tumorigenesis. USP7, also known as herpes virus-associated ubiquitin specific protease (HAUSP), was first discovered as a protein that plays a role in viral lytic growth. Interest in the enzyme intensified when USP7 was implicated in regulating degradation of the tumor suppressor p53, by stabilizing the major E3 ligase for p53, MDM2.

Consistent with its regulation of diverse substrates and biological processes USP7 has emerged as a drug target in a wide range of malignancies including multiple myeloma, breast cancer, neuroblastoma, glioma, and ovarian cancer. However, known USP7 inhibitors have been shown to exhibit modest potency against USP7 and poor selectivity over other DUBs. In addition to modest potency and selectivity, reported drawbacks of known USP7 inhibitor compounds include poor solubility and general toxicity. Therefore, there is a need for the development of more potent and selective irreversible USP7 inhibitors.

### SUMMARY OF THE DISCLOSURE

In some embodiments, the present disclosure relates to a compound of formula (I)

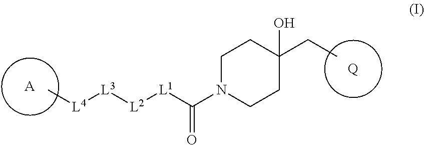

or a pharmaceutically acceptable salt thereof, wherein:
Q is

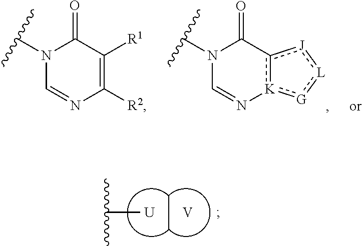

G is CR¹⁰, NR¹⁰, or N;
J is CR¹⁰, NR¹⁰, N, or S;
L is NR¹⁰, CR¹⁰, CR¹¹ or N;
K is C or N;
[symbol] is a single bond or a double bond, when valence permits, provided the ring containing J, L, and G is aromatic;
U is a 5-6 membered heterocyclyl optionally substituted with one or more Hal, —NH₂, —CN, —CF₃, —OH, oxo, or C₁₋₃ alkyl;
V is a 5-6 membered heteroaryl or a C₃₋₆ cycloalkyl, wherein the 5-6 membered heteroaryl or the C₅₋₆ cycloalkyl is optionally substituted with one or more Hal, —NH₂, —CN, —CF₃, —OH, oxo, or C₁₋₃ alkyl;
R¹ is H;
R² is H or a —NHC₁₋₃ alkylene(C₅₋₇ heterocyclyl),
   or R¹ and R² taken together with the carbon atoms to which they are attached form a C₆₋₁₀ aryl or a 5-6 membered heteroaryl, wherein the C₆₋₁₀ aryl or the 5-6 membered heteroaryl is optionally substituted with one or more substituents independently selected from Hal, C₁₋₃ alkyl, C₁₋₃ alkoxy, C₆₋₁₀ aryl, 5-6 membered heteroaryl, 4-7 membered heterocyclyl, —NHC(═O)C₁₋₃ alkylene(NR³R⁴), —C(═O)NHC₁₋₃ alkylene(NR³R⁴), —NHC₁₋₃ alkylene(4-7 membered heterocyclyl), —OC₁₋₃ alkylene(4-7 membered heterocyclyl), —OC₁₋₃ alkylene-O—(NR³R⁴), —NHC₁₋₃alkylene(NR³R⁴), or —OC₁₋₃ alkylene(NR³R⁴), and wherein the C₁₋₃ alkyl, the C₆₋₁₀ aryl, the 5-6 membered heteroaryl, or the 4-7 membered heterocyclyl is optionally independently substituted with one or more Hal, —NH₂, —CN, —CF₃, C₁₋₃ alkyl, or —C₀₋₂ alkyl(5-6 membered heterocyclyl);
R³ is a C₁₋₃ alkyl;
R⁴ is a C₁₋₃ alkyl,
   or R³ and R⁴ is together with the nitrogen atom to which they are attached form a 5-7 membered heterocyclyl or a 5-7 membered heteroaryl, wherein the 5-7 membered heterocyclyl is optionally substituted with one or more C₁₋₃ alkyl;
L¹ is a C₂₋₄ alkylene substituted with one or more —C₀₋₂ alkyl(C₆ aryl), —NHC₀₋₂ alkyl(C₆ aryl), or —C₀₋₂ alkyl(5-6 membered heteroaryl), wherein one or more carbons in the C₂₋₄ alkylene is optionally replaced with N, which is optionally substituted with C₁₋₃ alkyl; or L¹ is

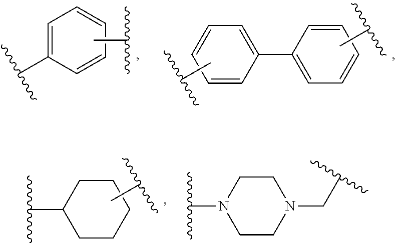

in either direction,

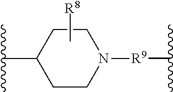

in either direction, or

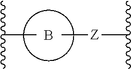

in either direction, wherein the —C₀₋₂ alkyl(C₆ aryl) or the —C₀₋₂ alkyl(5-6 membered heteroaryl) is optionally independently substituted with one or more Hal, —NH₂, —CN, —CF₃, or C₁₋₃ alkyl; Z is a C₁₋₄ alkylene, and [symbol] is a C₆₋₁₀ arylene, 5-7 membered heterocyclylene or a 5-7 membered heteroarylene, and wherein the C₁₋₄ alkylene, the 5-7 membered heterocyclylene, and the 5-7 membered heteroarylene is each optionally independently substituted with a C₀₋₂ alkyl(C₆ aryl) or a C₃₋₆ cycloalkyl;
L² is —NH—, a bond, or

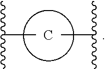

wherein [symbol] is a 5 membered heteroarylene;
L³ is —C(═O)—, —S(═O)₂—, a C₆₋₁₀ arylene, or a bond;
L⁴ is a C₁₋₃ alkylene, a C₂₋₄ alkenylene, or a bond;
[symbol] is H,

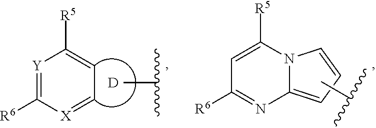

C₆₋₁₀ aryl, or a 5-7 membered heteroaryl, wherein the C₆₋₁₀ aryl or the 5-7 membered heteroaryl is optionally substituted with one or more Hal, —CN, a C₁₋₃ haloalkyl, —NHC(═O)C₁₋₃ alkenyl;
X is N or CH;
Y is N or CR⁷;
R⁵ is Hal, —NH₂, —NH₂(C₁₋₃ alkyl), —OH, a C₁₋₃ alkoxy, or C₁₋₃ alkyl;
R⁶ is H, Hal, —NH₂, —OH, —C(O)NH₂, a C₁₋₃ alkyl, a C₁₋₃ alkoxy, a C₆₋₁₀ aryl, a 5-6 membered heteroaryl, or a C₃₋₅ cycloalkyl, wherein —NH₂, —OH, the C₁₋₃ alkyl, the C₆₋₁₀ aryl, the 5-6 membered heteroaryl, or the C₃₋₅ cycloalkyl is optionally substituted with one or more Hal, —NH₂, —CN, —CF₃, a C₁₋₃ alkyl, C₁₋₃ alkyl(NH₂), or C₁₋₃ alkyl(CF₃);
R⁷ is H, Hal, a C₁₋₃ alkyl, C₁₋₃ haloalkyl, cyano, C₃₋₅ cycloalkyl, —C(O)NH₂, —C(O)N(C₁₋₃ alkyl)₂, —C(O)NHC₃₋₅ cycloalkyl, or —C(═O)NHC₁₋₃ alkyl, wherein when R⁷ is —C(O)N(C₁₋₃ alkyl)₂ the two C₁₋₃ alkyl groups are optionally taken together with the N atom to which they are attached to form a 3-7 membered heterocyclyl;
   or R⁷ and R⁶ taken together with the carbon atoms to which they are attached form a C₅₋₇ cycloalkyl, a C₆₋₈ bridged bicyclic cycloalkyl, or a 5-6 membered heterocyclyl, wherein the C₅₋₇ cycloalkyl or the 5-6 membered heterocyclyl is optionally substituted with one or more C₁₋₃ alkyl, oxo, or
   —C(═O)C₁₋₃ alkyl, wherein two C₁₋₃ alkyl on the same carbon are optionally taken together with the carbon atom to which they are attached to form a spiro ring;
[symbol] is a C₆₋₁₀ aryl, a 5-6 membered heterocyclyl, or a 5-6 membered heteroaryl;
R⁸ is a C₆₋₁₀ aryl, optionally substituted with one or more Hal, —NH₂, —CN, —CF₃, a C₁₋₃ alkyl, C₁₋₃ alkyl(NH₂), or C₁₋₃ alkyl(CF₃);

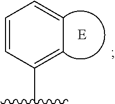

R⁹ is a bond, a C₂₋₄ alkylene, a C₆₋₁₀ arylene, or
[symbol] is a 5-6 membered heterocyclyl optionally substituted with one or more Hal, —NH₂, —CN, —CF₃, a C₁₋₃ alkyl, C₁₋₃ alkyl(NH₂), or C₁₋₃ alkyl(CF₃);
R¹⁰ is H, C₁₋₃ alkylene(C₅₋₇ heterocyclyl), or a C₆₋₁₀ aryl, wherein C₁₋₃ alkylene(C₅₋₇ heterocyclyl) or C₆₋₁₀ aryl is optionally substituted with one or more Hal, —NH₂, —CN, —CF₃, a C₁₋₃ alkyl, C₁₋₃ alkyl(NH₂), C₁₋₃ alkyl(CF₃) or C₁₋₃ alkyl(NH₂)(CF₃); and
R¹¹ is Hal, —NH₂, —CN, —CF₃, or C₁₋₃ alkyl, provided the compound is not

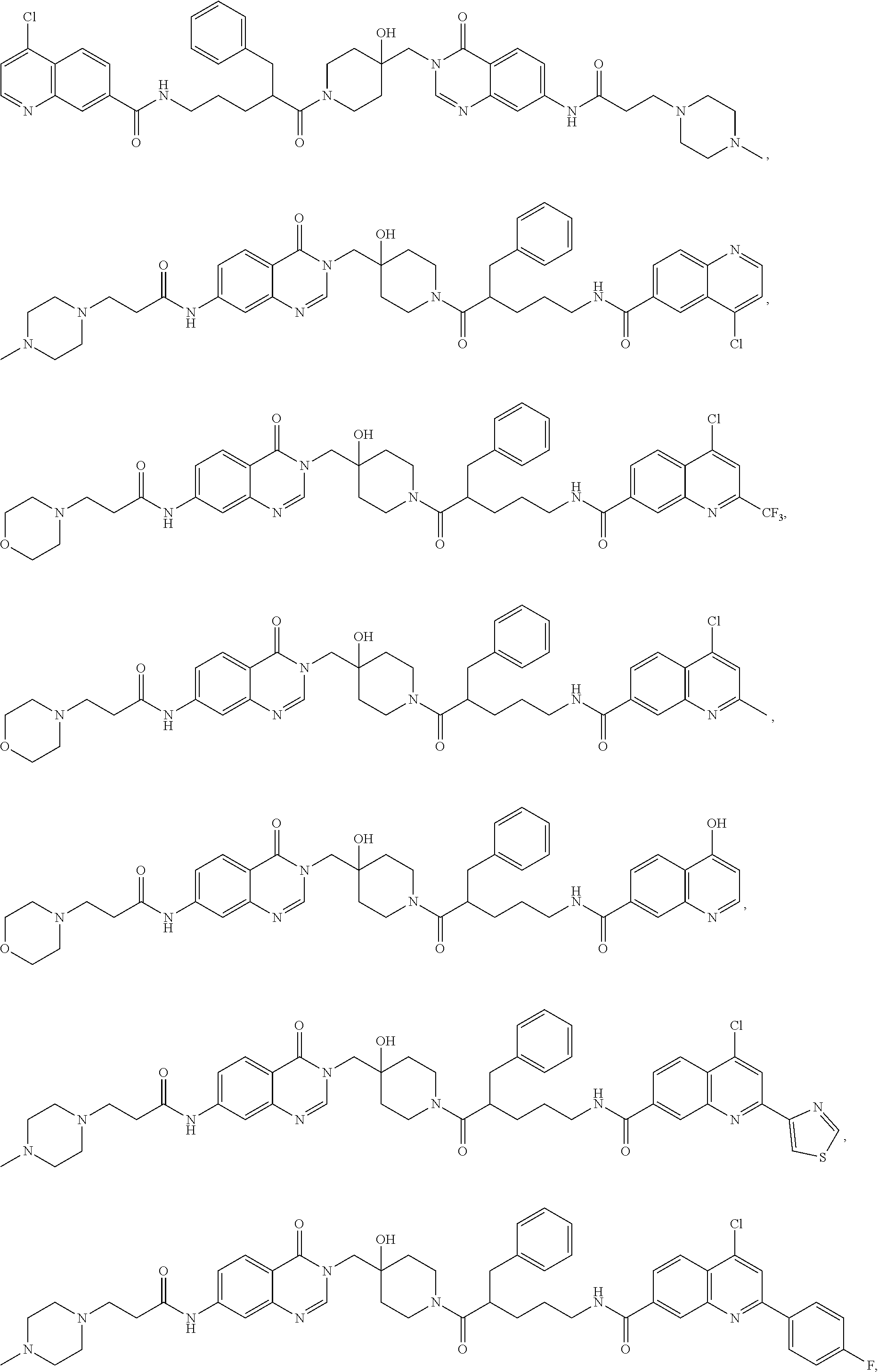

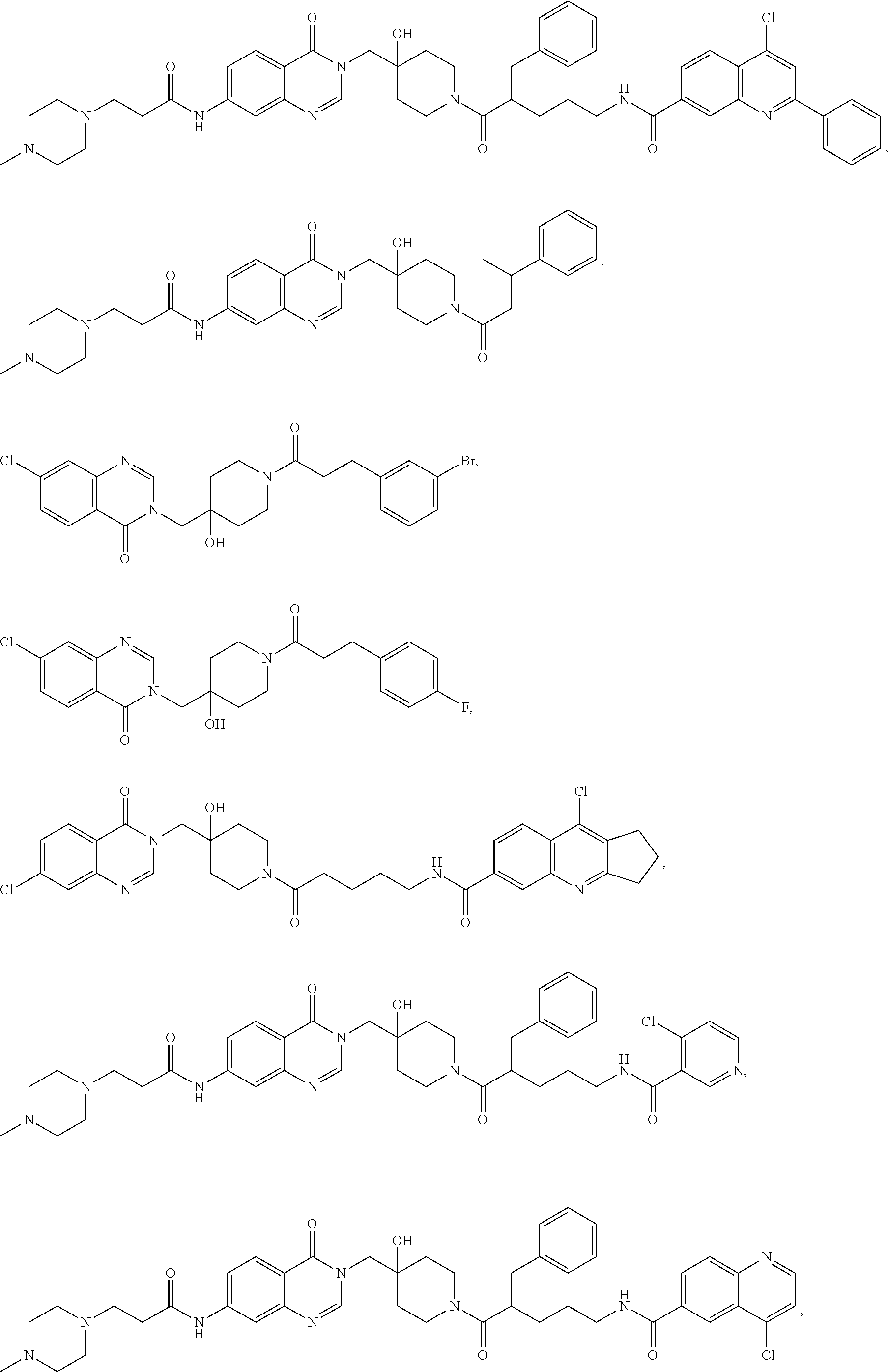

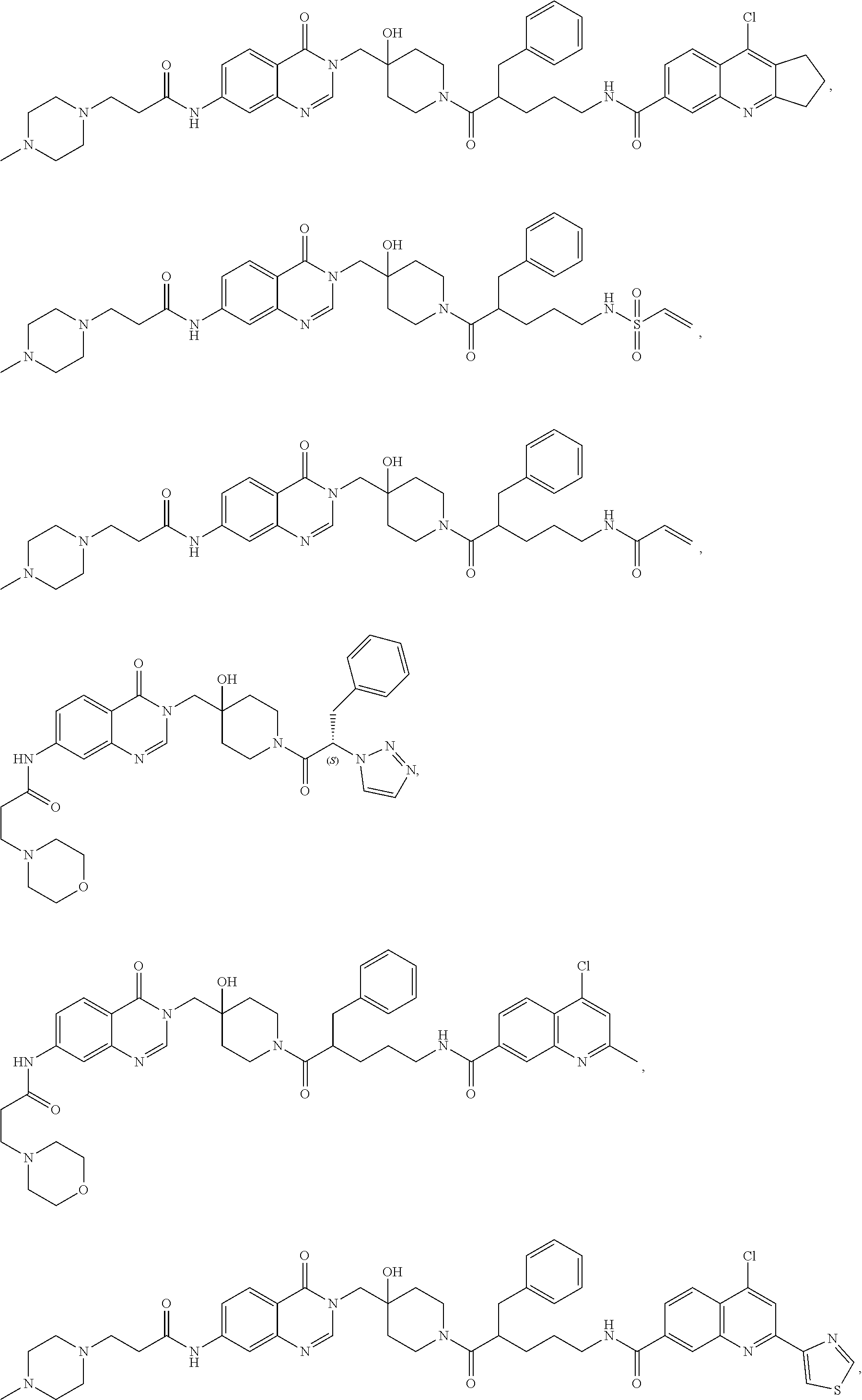

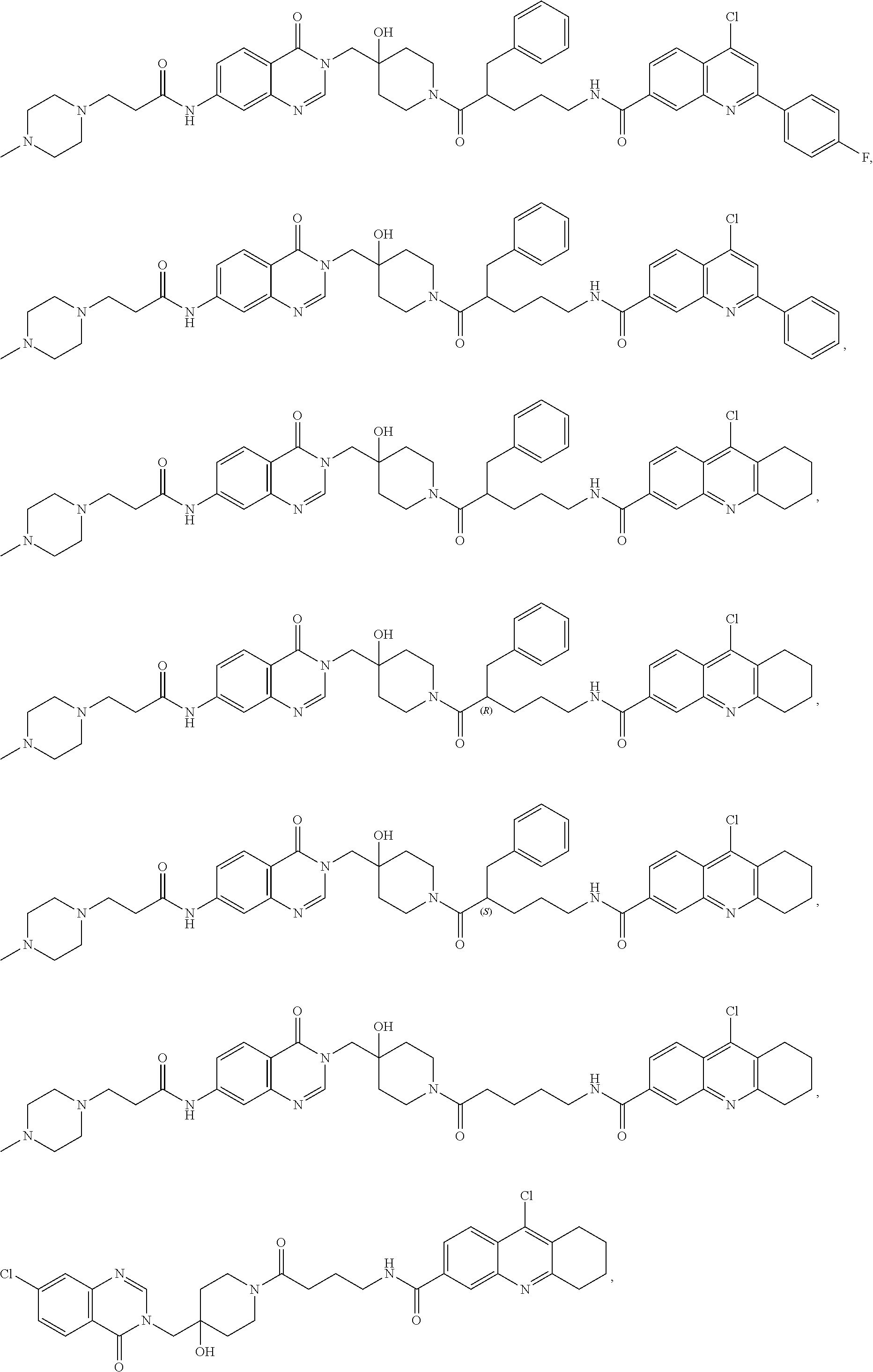

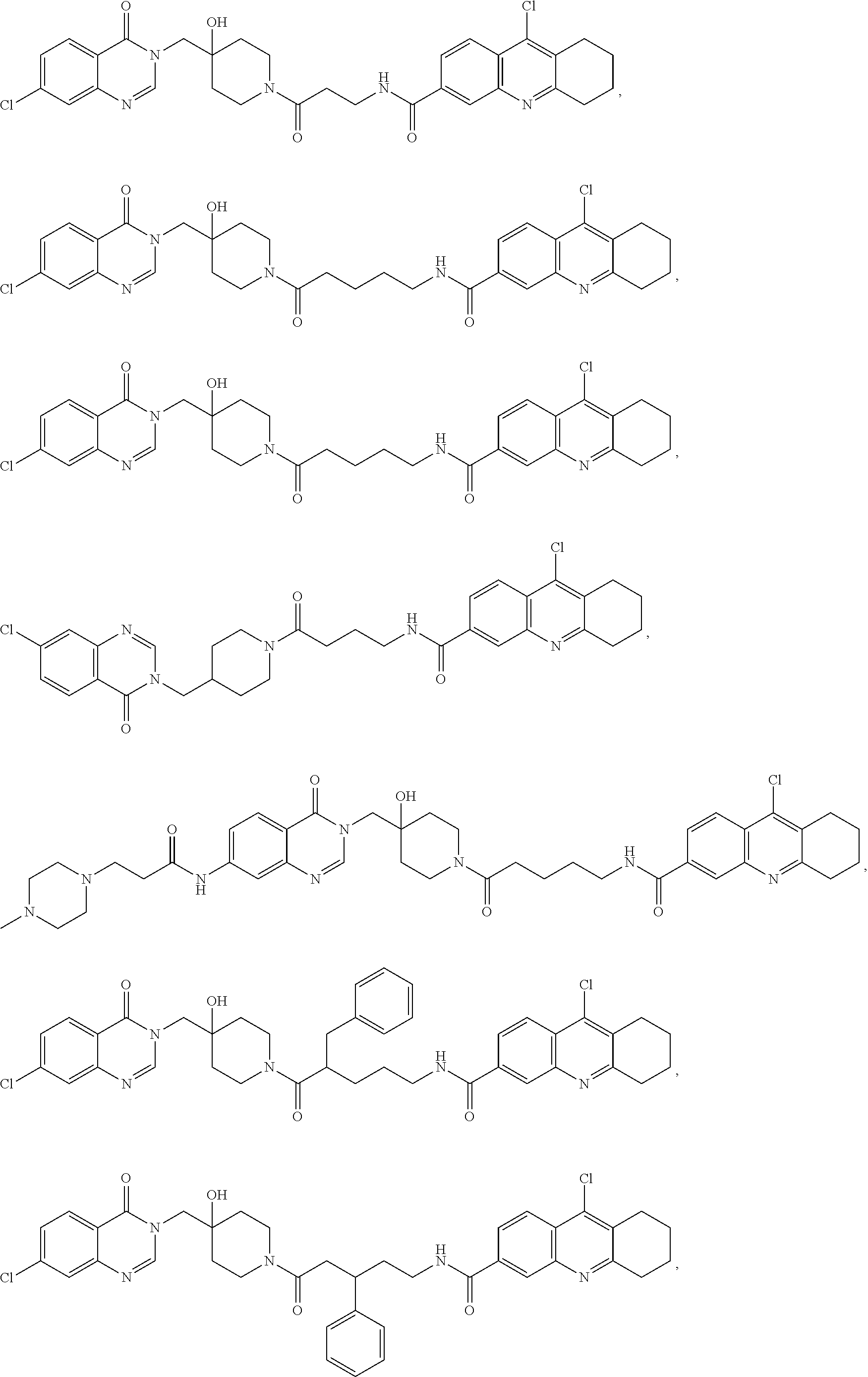

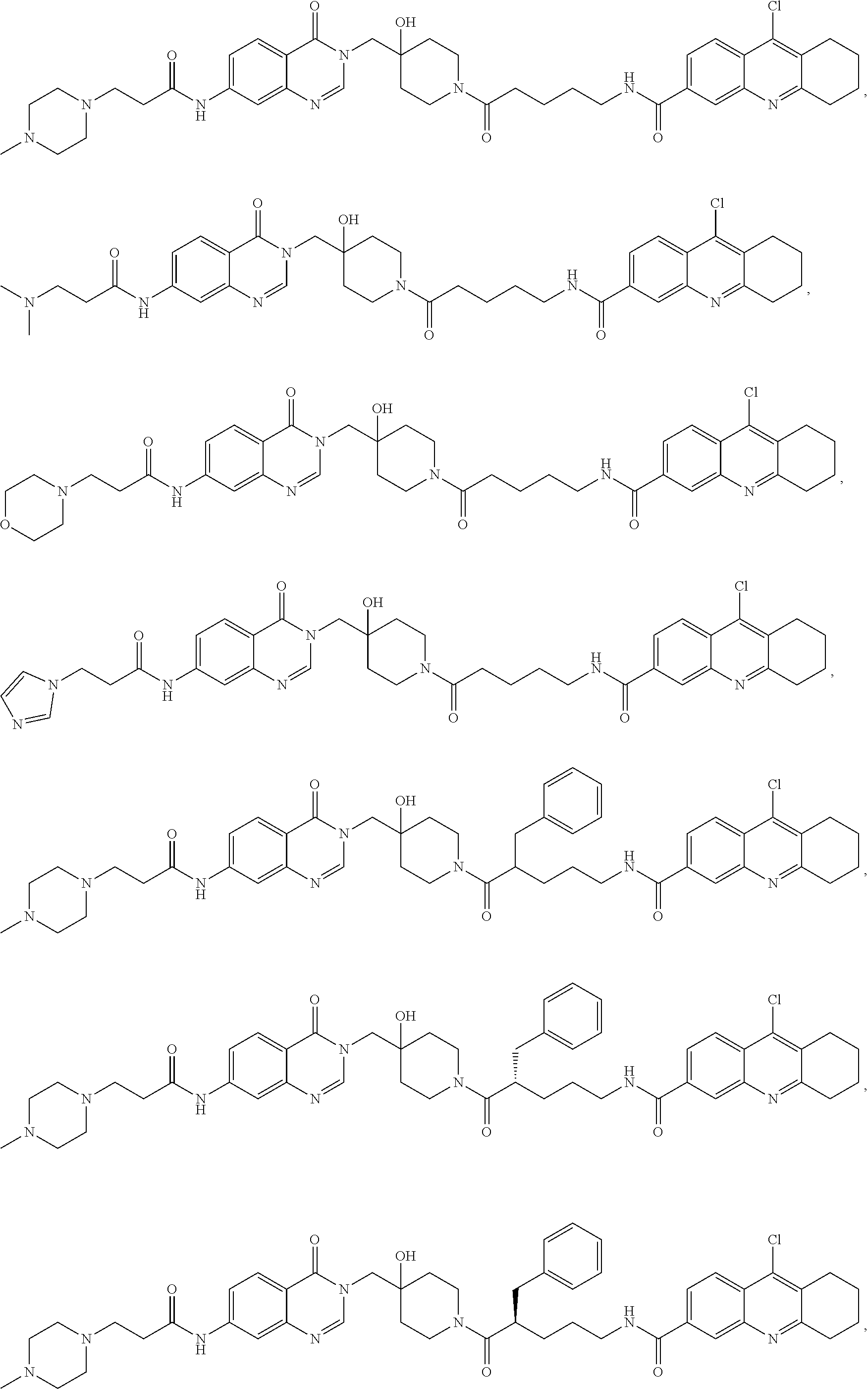

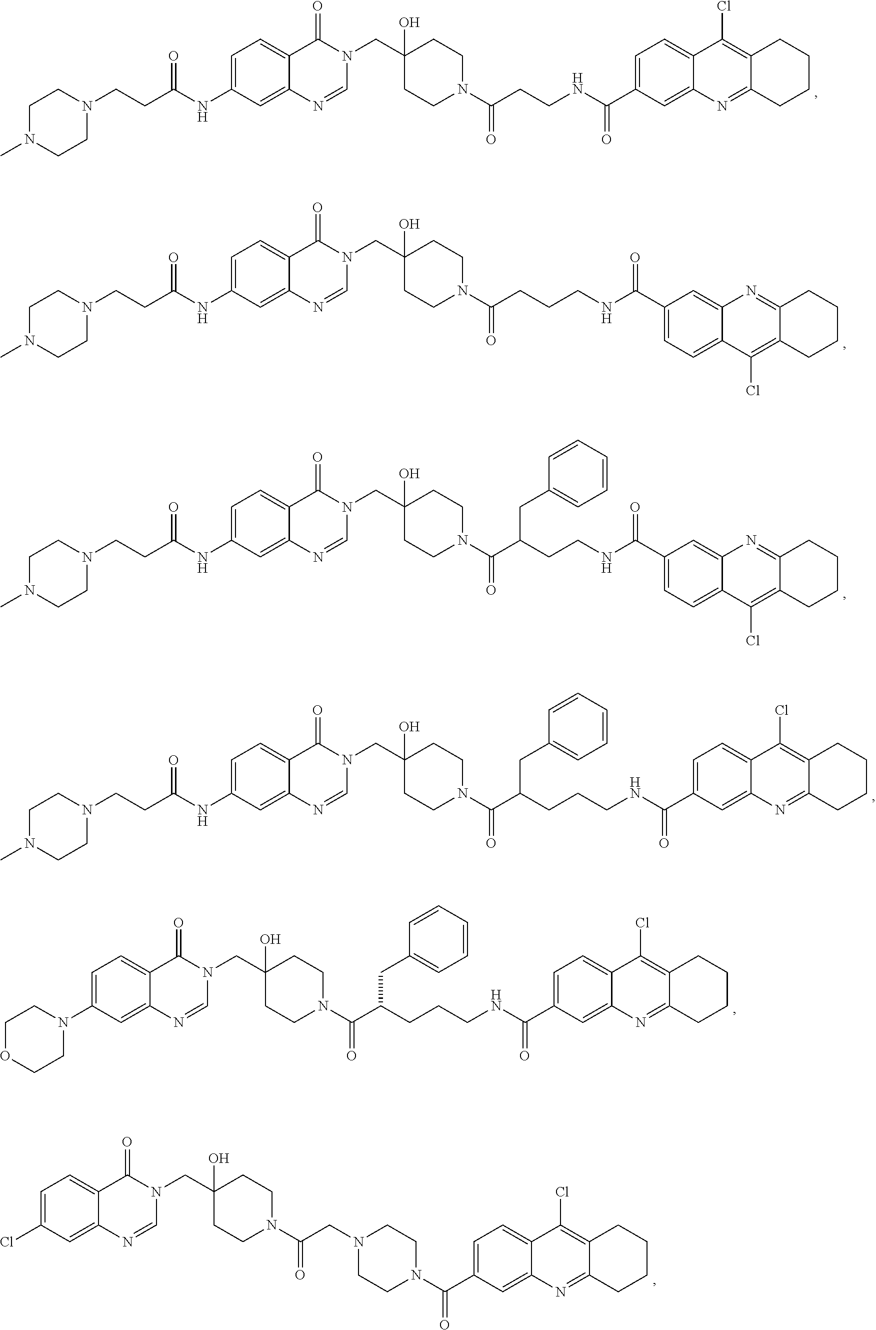

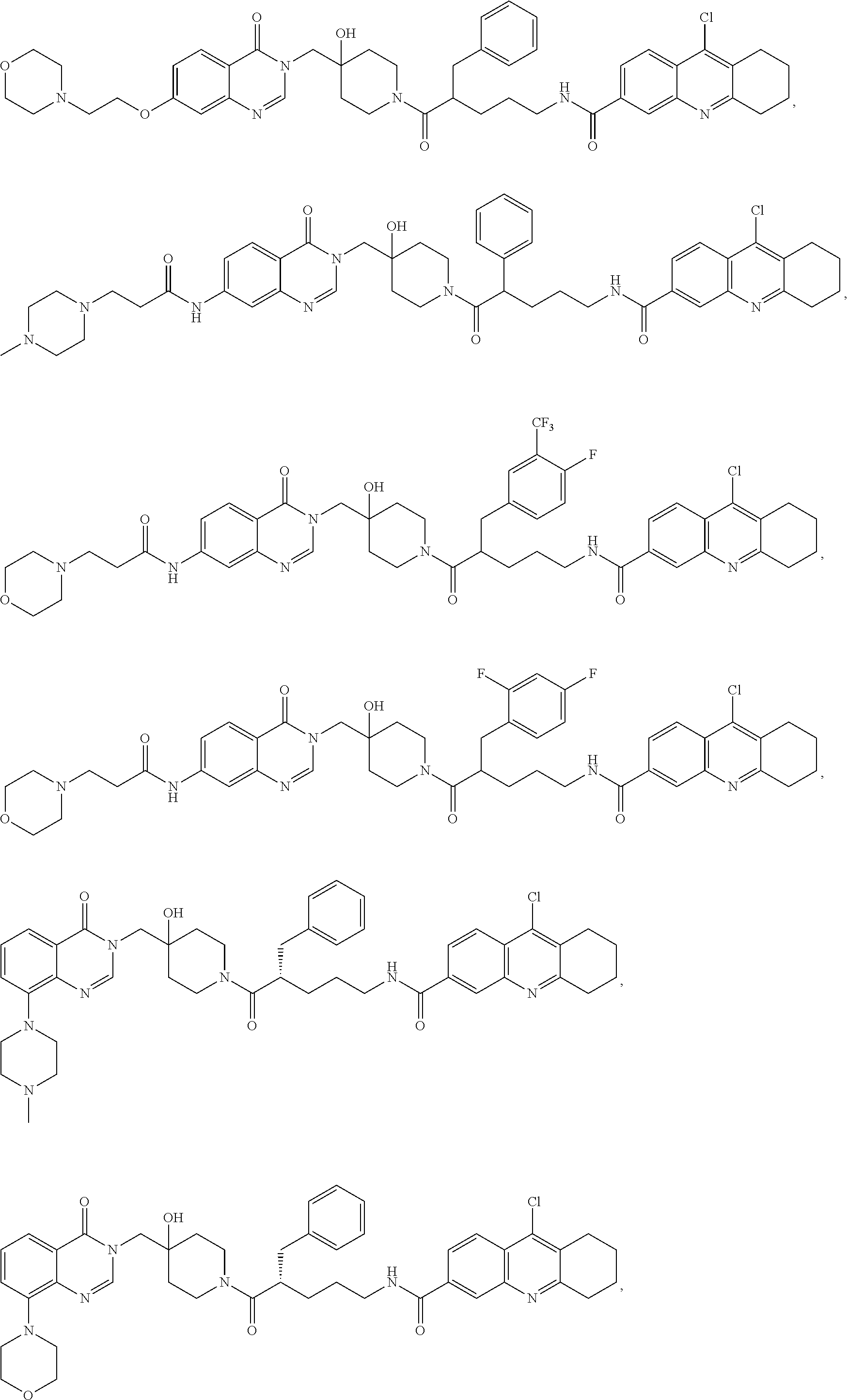

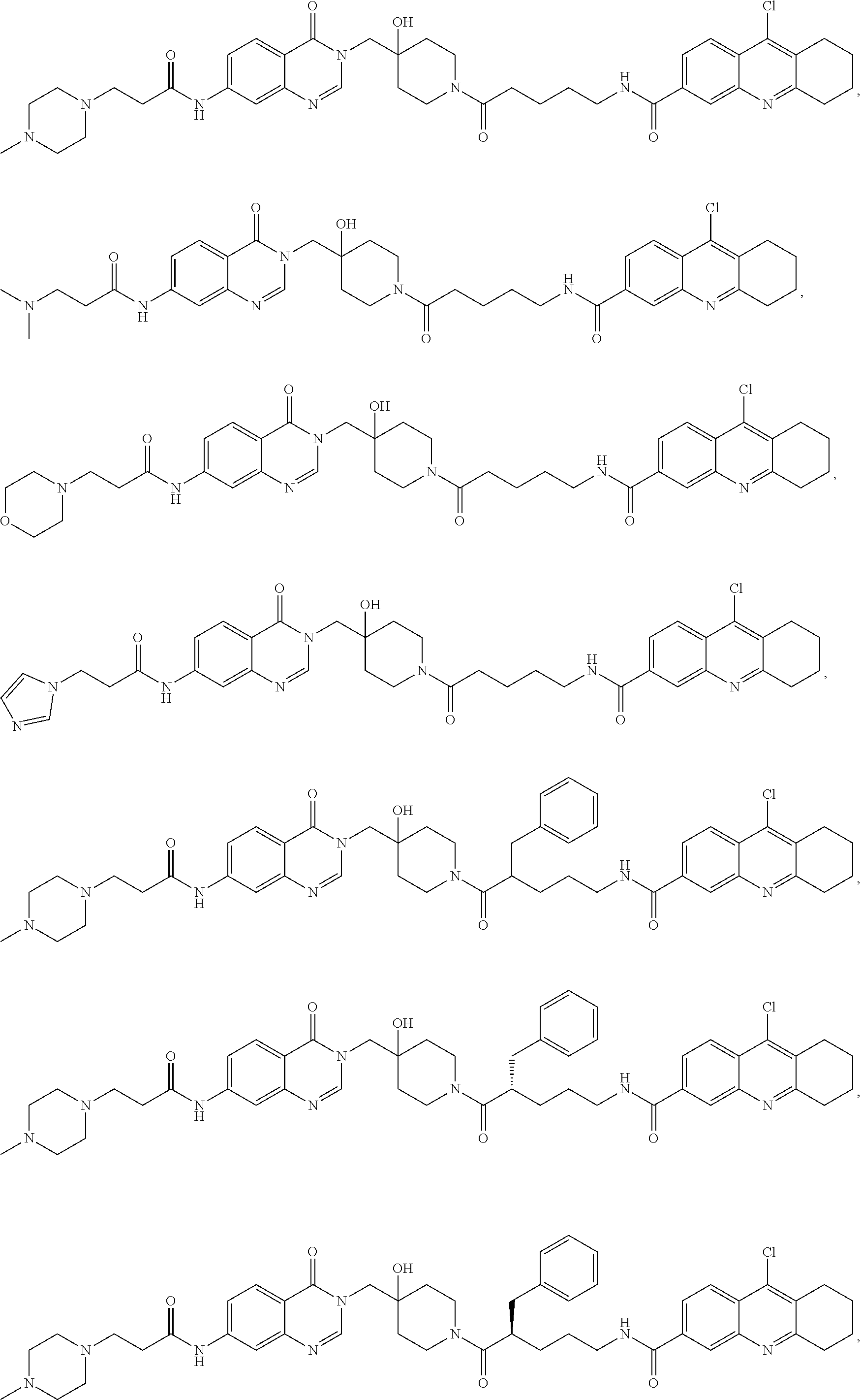

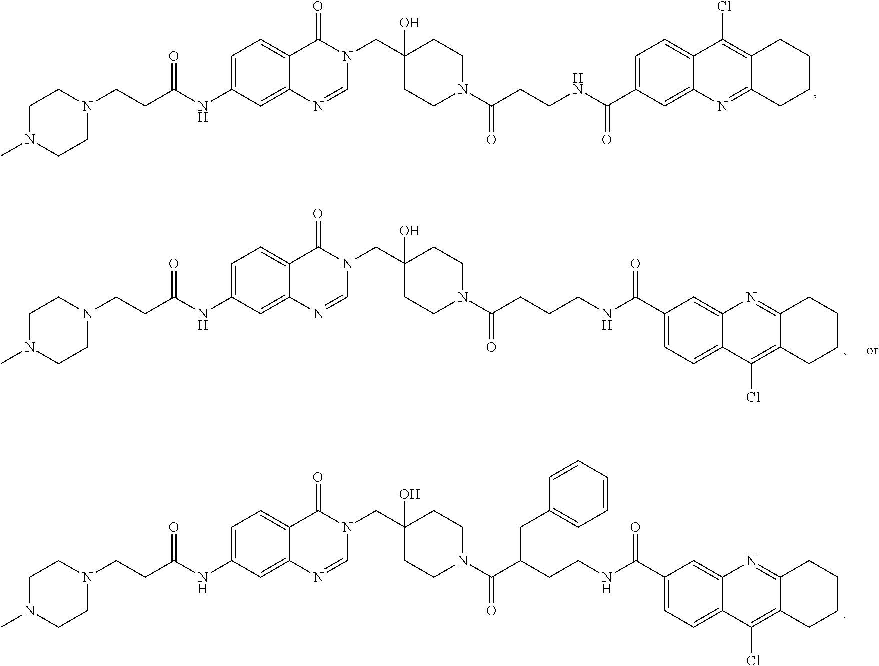 [SMILES: CN1CCN(CCC(=O)Nc2ccc3c(=O)n(CC4(O)CCN(C(=O)CCNC(=O)c5ccc6c(Cl)c7c(nc6c5)CCCC7)CC4)cnc3c2)CC1 ; CN1CCN(CCC(=O)Nc2ccc3c(=O)n(CC4(O)CCN(C(=O)CCCNC(=O)c5ccc6c(Cl)c7c(nc6c5)CCCC7)CC4)cnc3c2)CC1 ; CN1CCN(CCC(=O)Nc2ccc3c(=O)n(CC4(O)CCN(C(=O)C(CCNC(=O)c5ccc6c(Cl)c7c(nc6c5)CCCC7)Cc5ccccc5)CC4)cnc3c2)CC1]

In some embodiments, the present disclosure relates to a pharmaceutical composition comprising a compound of formula (I), such as a compound selected from compounds 4-144, and a pharmaceutically acceptable carrier.

In some embodiments, the present disclosure relates to a method of treating a disease or disorder modulated by USP7, comprising administering to a subject in need thereof a compound of formula (I), such as a compound selected from compounds 4-144, or a pharmaceutical composition comprising a compound of formula (I), such as a compound selected from compounds 4-144.

In some embodiments, the present disclosure relates to a method of inhibiting USP7, comprising administering to a subject in need thereof a compound of formula (I) or a pharmaceutical composition comprising a compound of formula (I).

In some embodiments, the present disclosure relates to a method of treating cancer, comprising administering to a subject in need thereof a compound of formula (I) or a pharmaceutical composition comprising a compound of formula (I).

In some embodiments, the present disclosure relates to a method of inhibiting USP7, wherein thereof a compound of formula (I) forms a covalent bond with USP7. In some embodiments, the covalent bond forms with a cysteine residue of USP7.

In some embodiments, the present disclosure relates to a use of thereof a compound of formula (I) for the manufacture of a medicament for treating a disease modulated by USP7.

In some embodiments, the present disclosure relates to thereof a compound of formula (I) for use in treating a disease modulated by USP7.

### DETAILED DESCRIPTION OF THE DISCLOSURE

USP7 (Ubiquitin Specific Protease 7)/HAUSP (Herpes Associated Ubiquitin Specific Protease) is a 135 kDa protein of the USP family. USP7 has been shown to interact with viral proteins, such as ICP0 (Vmw 110), a herpes simplex virus immediate-early gene stimulating initiation of the viral lytic cycle, and EBNA1 (Epstein-Barr Nuclear Antigen-1). The DUB USP7 has been shown to be involved in regulation of a myriad of cellular processes, including epigenetics, cell cycle, DNA repair, immunity, viral infection and tumorigenesis. Interest in the enzyme intensified when USP7 was implicated in regulating degradation of the tumor suppressor p53, by stabilizing the major E3 ligase for p53, MDM2. Consistent with recent reports, USP7 silencing has also been shown to increase steady-state p53 levels by promoting Mdm2 degradation. Binding of USP7 to p53 was recently shown to be regulated by TSPYL5, a protein potentially involved in breast oncogenesis through a competition with p53 for binding to the same region of USP7. More recently, both upregulation and downregulation of USP7 have been shown to inhibit colon cancer cell proliferation in vitro and tumor growth in vivo, by resulting in constitutively high p53 levels.

In some embodiments, the present disclosure relates to a compound of formula (I),

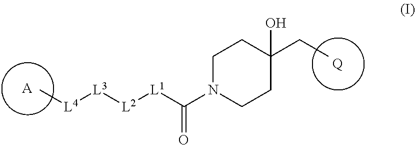

or a pharmaceutically acceptable salt thereof, wherein:
Q is

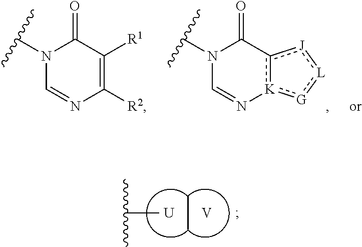

G is CR¹⁰, NR¹⁰, or N;
J is CR¹⁰, NR¹⁰, N, or S;
L is NR¹⁰, CR¹⁰, CR¹¹ or N;
K is C or N;
[symbol] is a single bond or a double bond, when valence permits, provided the ring containing J, L, and G is aromatic;
U is a 5-6 membered heterocyclyl optionally substituted with one or more Hal, —NH₂, —CN, —CF₃, —OH, oxo, or C₁₋₃ alkyl;
V is a 5-6 membered heteroaryl or a C₃₋₆ cycloalkyl, wherein the 5-6 membered heteroaryl or the C₅₋₆ cycloalkyl is optionally substituted with one or more Hal, —NH₂, —CN, —CF₃, —OH, oxo, or C₁₋₃ alkyl;
R¹ is H;
R² is a —NHC₁₋₃ alkylene(C₅₋₇ heterocyclyl),
   or R¹ and R² taken together with the carbon atoms to which they are attached form a C₆₋₁₀ aryl or a 5-6 membered heteroaryl, wherein the C₆₋₁₀ aryl or the 5-6 membered heteroaryl is optionally substituted with one or more substituents independently selected from Hal, C₁₋₃ alkyl, C₁₋₃ alkoxy, C₆₋₁₀ aryl, 5-6 membered heteroaryl, 4-7 membered heterocyclyl, —NHC(═O)C₁₋₃ alkylene(NR³R⁴), —C(═O)NHC₁₋₃ alkylene(NR³R⁴), —NHC₁₋₃ alkylene(4-7 membered heterocyclyl), —OC₁₋₃ alkylene(4-7 membered heterocyclyl), —OC₁₋₃ alkylene-O—(NR³R⁴), or —OC₁₋₃ alkylene(NR³R⁴), and wherein the C₁₋₃ alkyl, the C₆₋₁₀ aryl, the 5-6 membered heteroaryl, or the 4-7 membered heterocyclyl is optionally independently substituted with one or more Hal, —NH₂, —CN, —CF₃, C₁₋₃ alkyl, or —C₀₋₂ alkyl(5-6 membered heterocyclyl);
R³ is a C₁₋₃ alkyl;
R⁴ is a C₁₋₃ alkyl,
   or R³ and R⁴ is together with the nitrogen atom to which they are attached form a 5-7 membered heterocyclyl or a 5-7 membered heteroaryl, wherein the 5-7 membered heterocyclyl is optionally substituted with one or more C₁₋₃ alkyl;
L¹ is a C₂₋₄ alkylene substituted with one or more —C₀₋₂ alkyl(C₆ aryl), —NHC₀₋₂ alkyl(C₆ aryl), or —C₀₋₂ alkyl(5-6 membered heteroaryl),

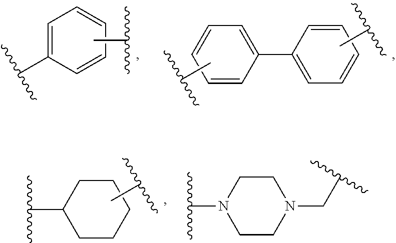

in either direction,

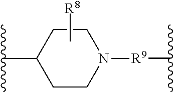

in either direction, or

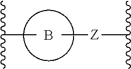

in either direction, wherein the —C₀₋₂ alkyl(C₆ aryl) or the —C₀₋₂ alkyl(5-6 membered heteroaryl) is optionally independently substituted with one or more Hal, —NH₂, —CN, —CF₃, or C₁₋₃ alkyl; Z is a C₁₋₄ alkylene, and [symbol] is a C₆₋₁₀ arylene, 5-7 membered heterocyclylene or a 5-7 membered heteroarylene, and wherein the C₁₋₄ alkylene, the 5-7 membered heterocyclylene, and the 5-7 membered heteroarylene is each optionally independently substituted with a C₀₋₂ alkyl(C₆ aryl) or a C₃₋₆ cycloalkyl;
L² is —NH—, a bond, or

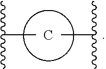

wherein [symbol] is a 5 membered heteroarylene;
L³ is —C(═O)—, —S(═O)₂—, a C₆₋₁₀ arylene, or a bond;
L⁴ is a C₁₋₃ alkylene, a C₂₋₄ alkenylene, or a bond;
[symbol] is H,

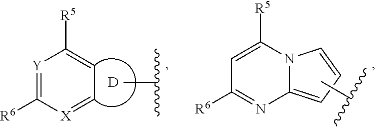

a C₆₋₁₀ aryl, or a 5-7 membered heteroaryl, wherein the C₆₋₁₀ aryl or the 5-7 membered heteroaryl is optionally substituted with one or more Hal, —CN, a C₁₋₃ haloalkyl, —NHC(═O)C₁₋₃ alkenyl;
X is N or CH;
Y is N or CR⁷;
R⁵ is Hal, —NH₂, —NH₂(C₁₋₃ alkyl), —OH, a C₁₋₃ alkoxy, or C₁₋₃ alkyl;
R⁶ is H, Hal, —NH₂, —OH, —C(O)NH₂, a C₁₋₃ alkyl, a C₁₋₃ alkoxy, a C₆₋₁₀ aryl, a 5-6 membered heteroaryl, or a C₃₋₅ cycloalkyl, wherein —NH₂, —OH, the C₁₋₃ alkyl, the C₆₋₁₀ aryl, the 5-6 membered heteroaryl, or the C₃₋₅ cycloalkyl is optionally substituted with one or more Hal, —NH₂, —CN, —CF₃, a C₁₋₃ alkyl, C₁₋₃ alkyl(NH₂), or C₁₋₃ alkyl(CF₃);
R⁷ is H, Hal, a C₁₋₃ alkyl, C₁₋₃ haloalkyl, or —C(═O)NHC₁₋₃ alkyl,
   or R⁷ and R⁶ taken together with the carbon atoms to which they are attached form a C₅ cycloalkyl, a C₆₋₈ bridged bicyclic cycloalkyl, or a 5-6 membered heterocyclyl, wherein the C₅ cycloalkyl or the 5-6 membered heterocyclyl is optionally substituted with one or more C₁₋₃ alkyl, oxo, or —C(═O)C₁₋₃ alkyl;
[symbol] is a C₆₋₁₀ aryl, a 5-6 membered heterocyclyl, or a 5-6 membered heteroaryl;
R⁸ is a C₆₋₁₀ aryl, optionally substituted with one or more Hal, —NH₂, —CN, —CF₃, a C₁₋₃ alkyl, C₁₋₃ alkyl(NH₂), or C₁₋₃ alkyl(CF₃);
R⁹ is a bond, a C₂₋₄ alkylene, a C₆₋₁₀ arylene, or

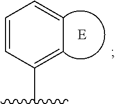

[symbol] is a 5-6 membered heterocyclyl optionally substituted with one or more Hal, —NH₂, —CN, —CF₃, a C₁₋₃ alkyl, C₁₋₃ alkyl(NH₂), or C₁₋₃ alkyl(CF₃);
R¹⁰ is H, C₁₋₃ alkylene(C₅₋₇ heterocyclyl), or a C₆₋₁₀ aryl, wherein C₁₋₃ alkylene(C₅₋₇ heterocyclyl) or C₆₋₁₀ aryl is optionally substituted with one or more Hal, —NH₂, —CN, —CF₃, a C₁₋₃ alkyl, C₁₋₃ alkyl(NH₂), C₁₋₃ alkyl(CF₃) or C₁₋₃ alkyl(NH₂)(CF₃); and
R¹¹ is Hal, —NH₂, —CN, —CF₃, or C₁₋₃ alkyl, provided the compound is not

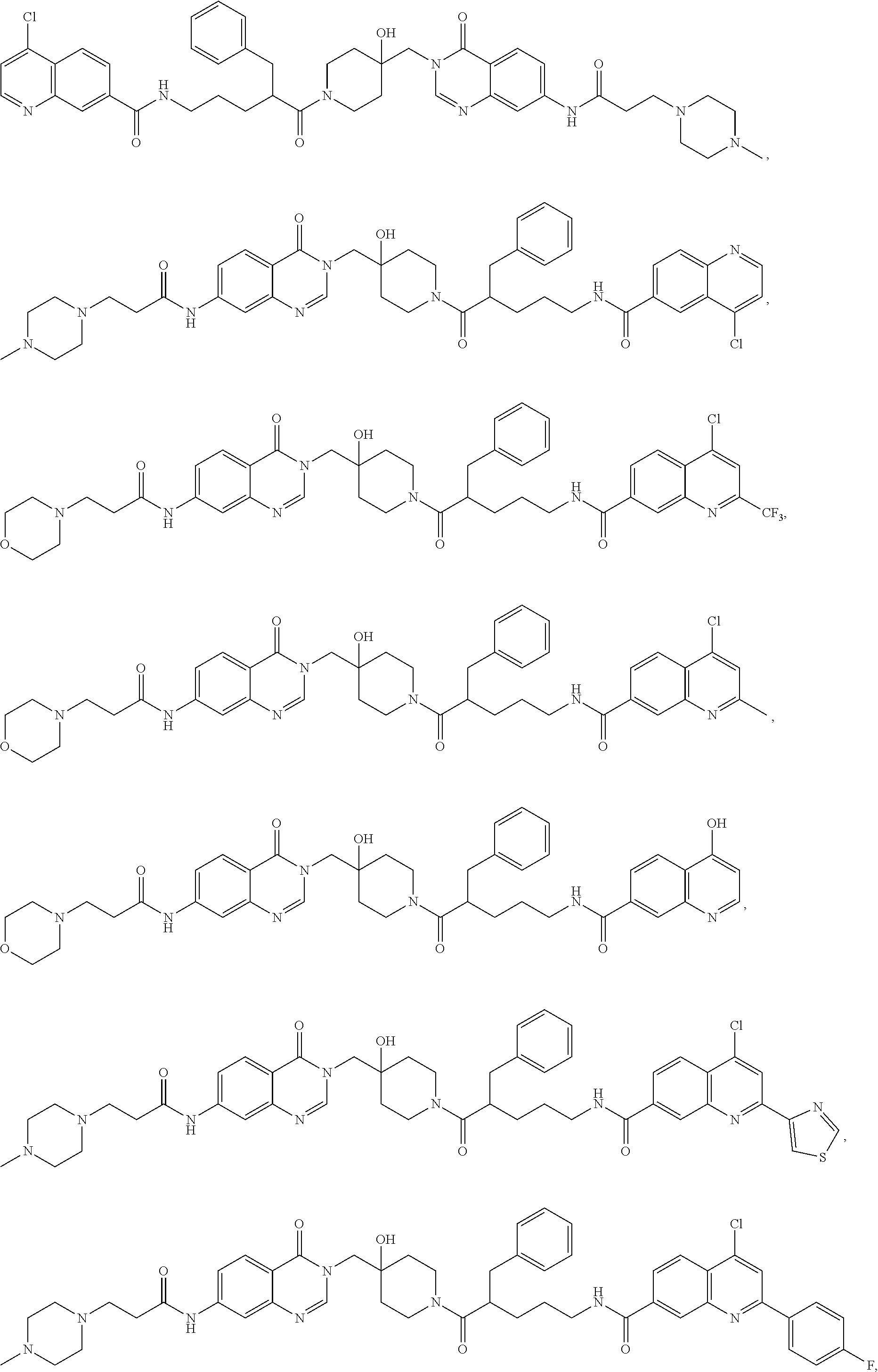

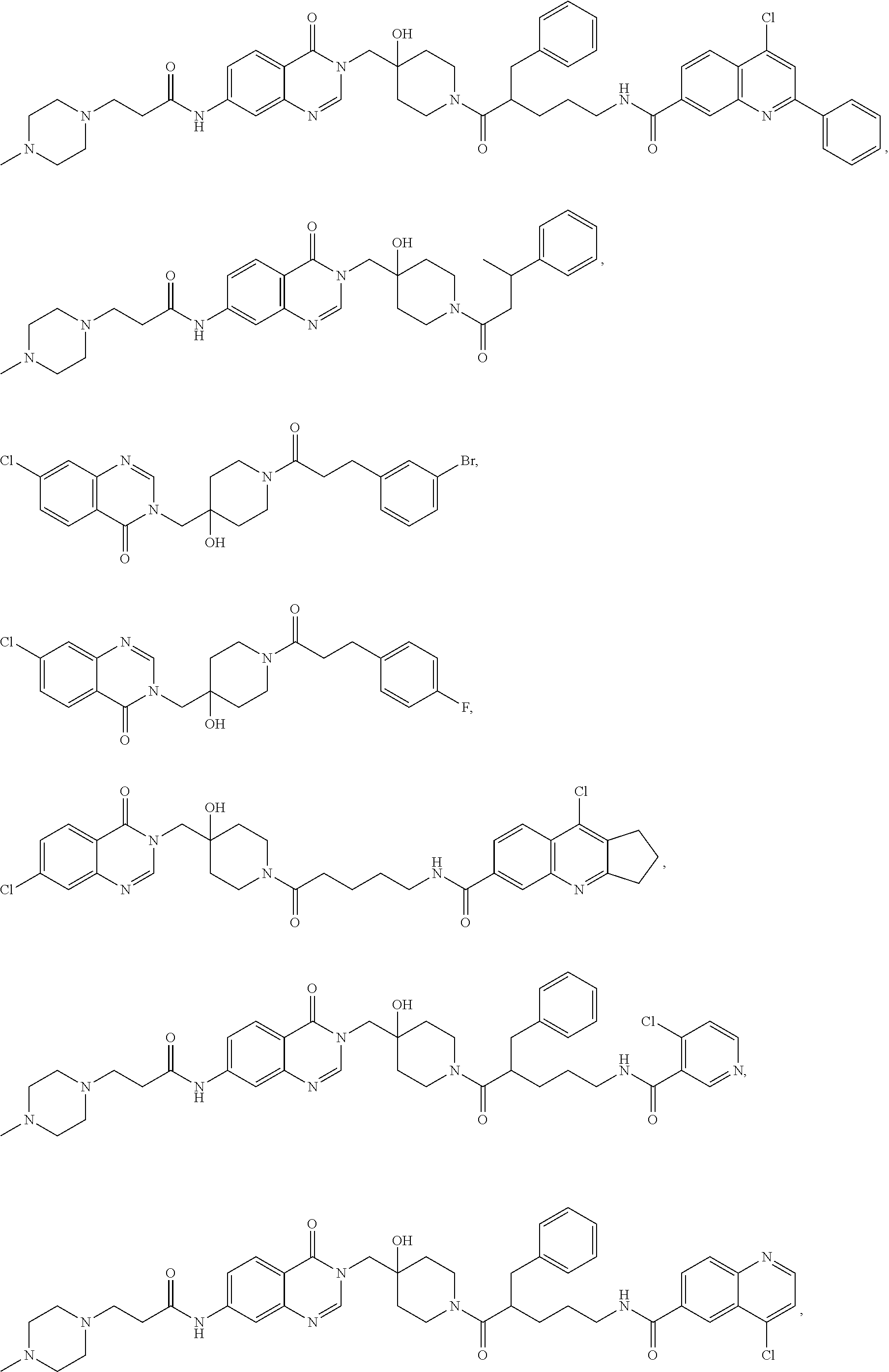

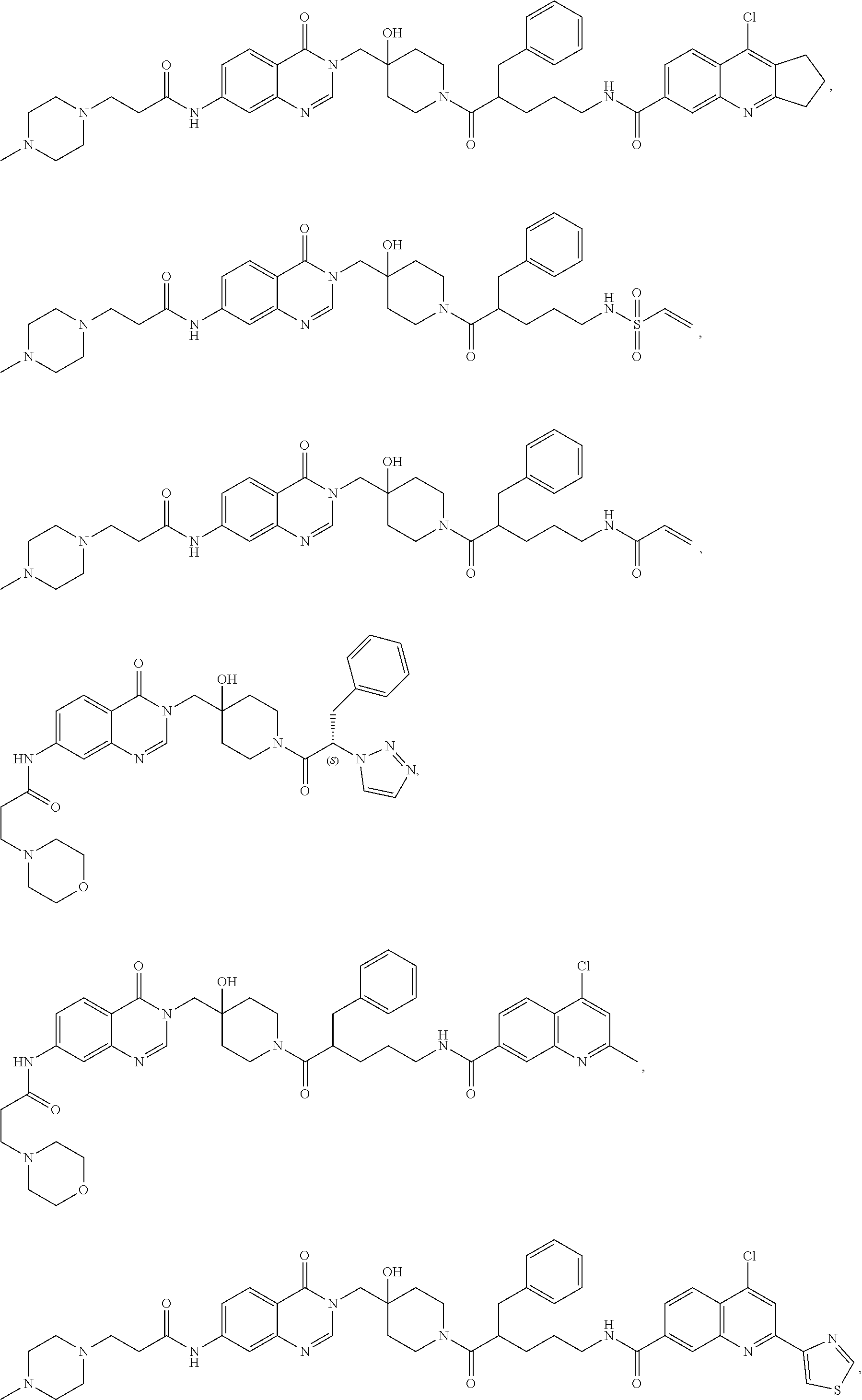

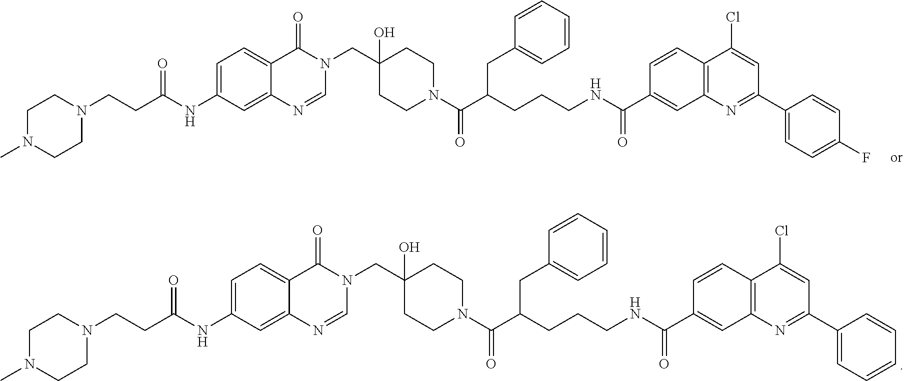

In some embodiments, Q is

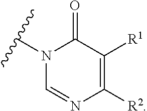

In some embodiments, Q is

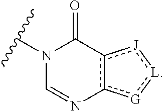

In some embodiments, Q is

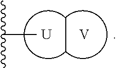

In some embodiments, L is

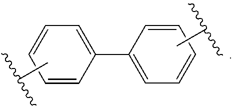

In some embodiments, L¹ is

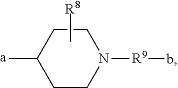

wherein a is the point of attachment to the carbonyl group and b is the point of attachment to L². In some embodiments, L¹ is

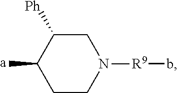

wherein a is the point of attachment to the carbonyl group and b is the point of attachment to L². In some embodiments, L¹ is

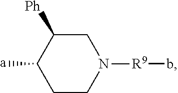

wherein a is the point of attachment to the carbonyl group and b is the point of attachment to L².

In some embodiments, R⁹ is a C₂₋₄ alkylene. In some embodiments, R⁹ is a C₆₋₁₀ arylene.

In some embodiments, L² is —NH—.

In some embodiments, L³ is —S(═O)₂—. In some embodiments, L³ is —C(═O)—.

In some embodiments, [symbol] is H and L⁴ is a C₁₋₃ alkyl or a C₃₋₄ alkenyl. In some embodiments, L⁴ is C₃₋₄ alkenyl. In some embodiments, [symbol] is

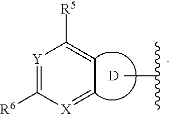

In some embodiments, [symbol] is a 5-6 membered heterocyclyl or a 5-6 membered heteroaryl. In some embodiments, [symbol] is

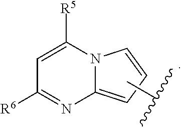

In some embodiments, the compound of Formula (I) is a compound of Formula (Ia):

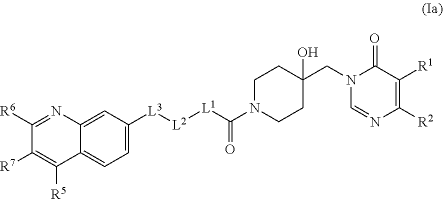

or a pharmaceutically acceptable salt thereof.

In some embodiments, L² is —NH—. In some embodiments, L² is

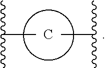

In some embodiments, L³ is —C(═O)—.

In some embodiments, the compound of Formula (I) is a compound of Formula (Ib):

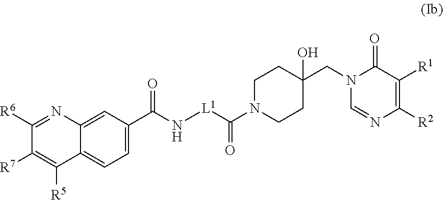

or a pharmaceutically acceptable salt thereof.

In some embodiments, R⁵ is Hal, such as R⁵ is C₁. In some embodiments, R⁵ is —OH. In some embodiments, R⁵ is Me.

In some embodiments, R⁷ is H.

In some embodiments, R⁶ is H. In some embodiments, R⁶ is F, —NH₂, or —OH, and the —NH₂ or the —OH is optionally substituted with a C₁₋₃ alkyl. In some embodiments, R⁶ is a C₁₋₃ alkyl, and the C₁₋₃ alkyl is optionally substituted with one or more Hal or —NH₂. In some embodiments, R⁶ is a C₆₋₁₀ aryl, and the C₆₋₁₀ aryl is optionally substituted with one or more Hal, —NH₂, —CN, —CF₃, a C₁₋₃ alkyl, C₁₋₃ alkyl(NH₂), or C₁₋₃ alkyl(CF₃). In some embodiments, R⁶ is a 5-6 membered heteroaryl. In some embodiments, R⁶ is a C₃₋₅ cycloalkyl, and the C₃₋₅ cycloalkyl is optionally substituted with one or more Hal.

In some embodiments, R⁷ and R⁶ taken together with the carbon atoms to which they are attached form a C₅ cycloalkyl or a 5-6 membered heterocyclyl, and the C₅ cycloalkyl or the 5-6 membered heterocyclyl is optionally substituted with one or more C₁₋₃ alkyl, oxo, or —C(═O)C₁₋₃ alkyl. In some embodiments, R⁷ and R⁶ taken together with the carbon atoms to which they are attached form a 5-6 membered heterocyclyl, and the 5-6 membered heterocyclyl is optionally substituted with one or more C₁₋₃ alkyl, oxo, or —C(═O)C₁₋₃ alkyl. In some embodiments, R⁷ and R⁶ taken together with the carbon atoms to which they are attached form a C₅ cycloalkyl.

In some embodiments, L¹ is a C₂₋₄ alkylene substituted with one or more —C₀₋₂ alkyl(C₆ aryl). In some embodiments, the one or more —C₀₋₂alkyl(C₆ aryl) is substituted with one or more Hal, —NH₂, —CN, —CF₃, or C₁₋₃ alkyl. In some embodiments, L¹ is a C₂₋₄ alkylene substituted with —CH₂Ph. In some embodiments, L¹ is

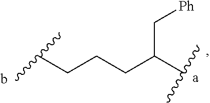

wherein a is the point of attachment to the carbonyl group and b is the point of attachment to L². In some embodiments, L¹ is

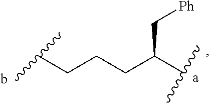

wherein a is the point of attachment to the carbonyl group and b is the point of attachment to L². In some embodiments, L¹ is a

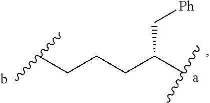

wherein a is the point of attachment to the carbonyl group and b is the point of attachment to L². In some embodiments, L¹ is

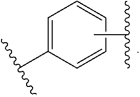

In some embodiments, L¹ is

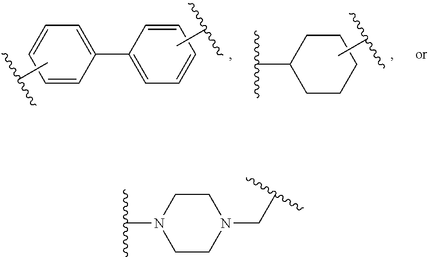

in either direction. In some embodiments, L¹ is

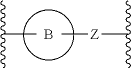

in either direction. In some embodiments, L¹ is

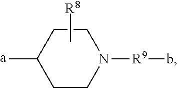

wherein a is the point of attachment to the carbonyl group and b is the point of attachment to L². In some embodiments, L¹ is

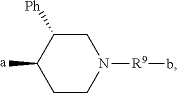

wherein a is the bond to the carbonyl group and b is the bond to L². In some embodiments, L¹ is

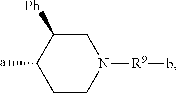

wherein a is the point of attachment to the carbonyl group and b is the point of attachment to L².

In some embodiments, R⁹ is a C₂₋₄ alkylene. In some embodiments, R⁹ is a C₆₋₁₀ arylene. In some embodiments, R⁹ is

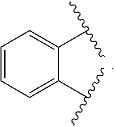 [SMILES: *c1ccccc1*]

In some embodiments, L² is a bond. In some embodiments, L² is —NH—. In some embodiments, L²

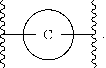

In some embodiments, L³ is —C(═O)—.

In some embodiments, R¹ is H.

In some embodiments, R² is a —NHC₁₋₃ alkylene(C₅₋₇ heterocyclyl). In some embodiments, R¹ and R² taken together with the carbon atoms to which they are attached form a C₆₋₁₀ aryl. In some embodiments, the C₆₋₁₀ aryl is substituted with one or more —NHC(═O)C₁₋₃ alkylene(NR³R⁴). In some embodiments, the C₆₋₁₀ aryl is substituted with one or more —NHC(═O)C₂alkylene(NMe₂). In some embodiments, the C₆₋₁₀ aryl is substituted with one or more —OC₁₋₃ alkylene(NR³R⁴)

In some embodiments, each R³ and R⁴ is independently a C₁₋₃ alkyl. In some embodiments, each R³ and R⁴ is Me. In some embodiments, R³ and R⁴ together with the nitrogen atom to which they are attached form a 5-7 membered heterocyclyl. In some embodiments, the 5-7 membered heterocyclyl is substituted with one or more C₁₋₃ alkyl. In some embodiments, R³ and R⁴ together with the nitrogen atom to which they are attached form a 5-7 membered heteroaryl.

In some embodiments, R¹ and R² taken together with the carbon atoms to which they are attached form a 5-6 membered heteroaryl. In some embodiments, the 5-6 membered heteroaryl is substituted with a one or more C₆₋₁₀ aryl. In some embodiments, the C₆₋₁₀ aryl is substituted with one or more Hal, —NH₂, —CN, —CF₃, or C₁₋₃ alkyl.

In some embodiments, the compound of Formula (I) is a compound of Formula (Ic) or a compound of Formula (Id):

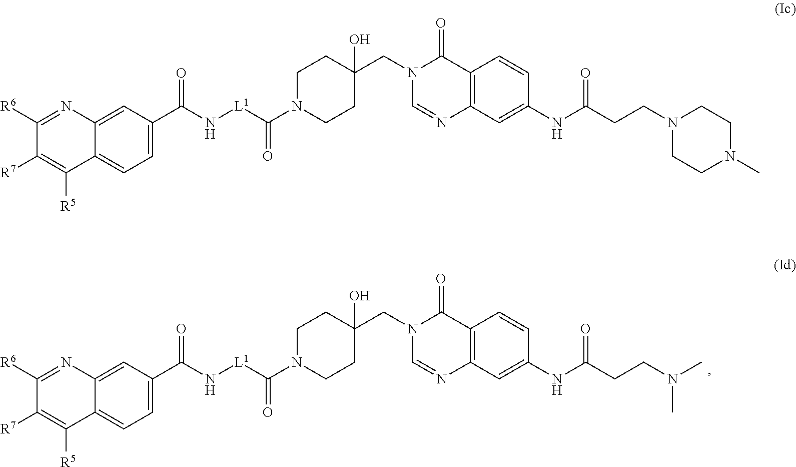

or a pharmaceutically acceptable salt thereof.

In some embodiments, the compound of formula (I) is not

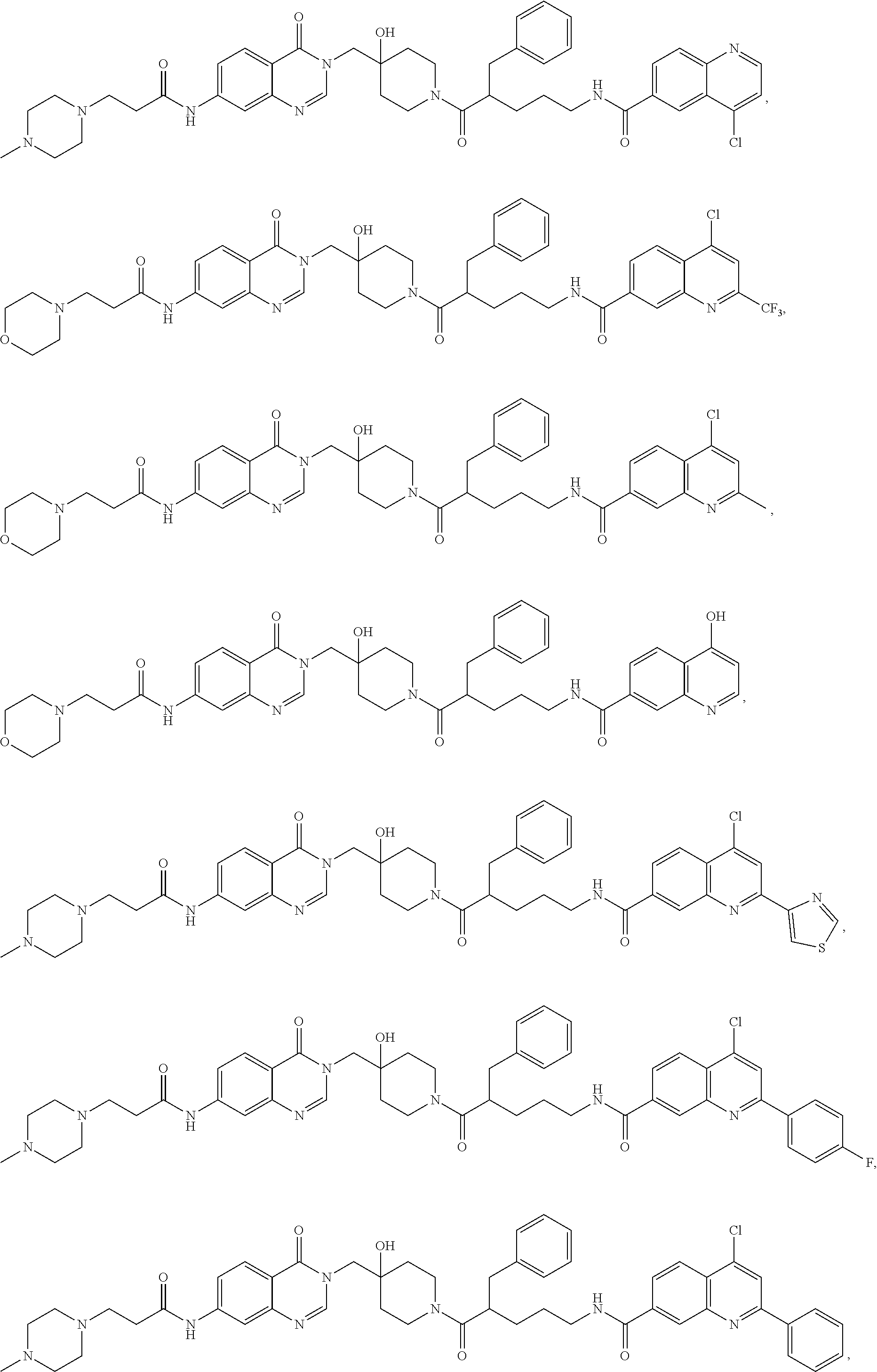

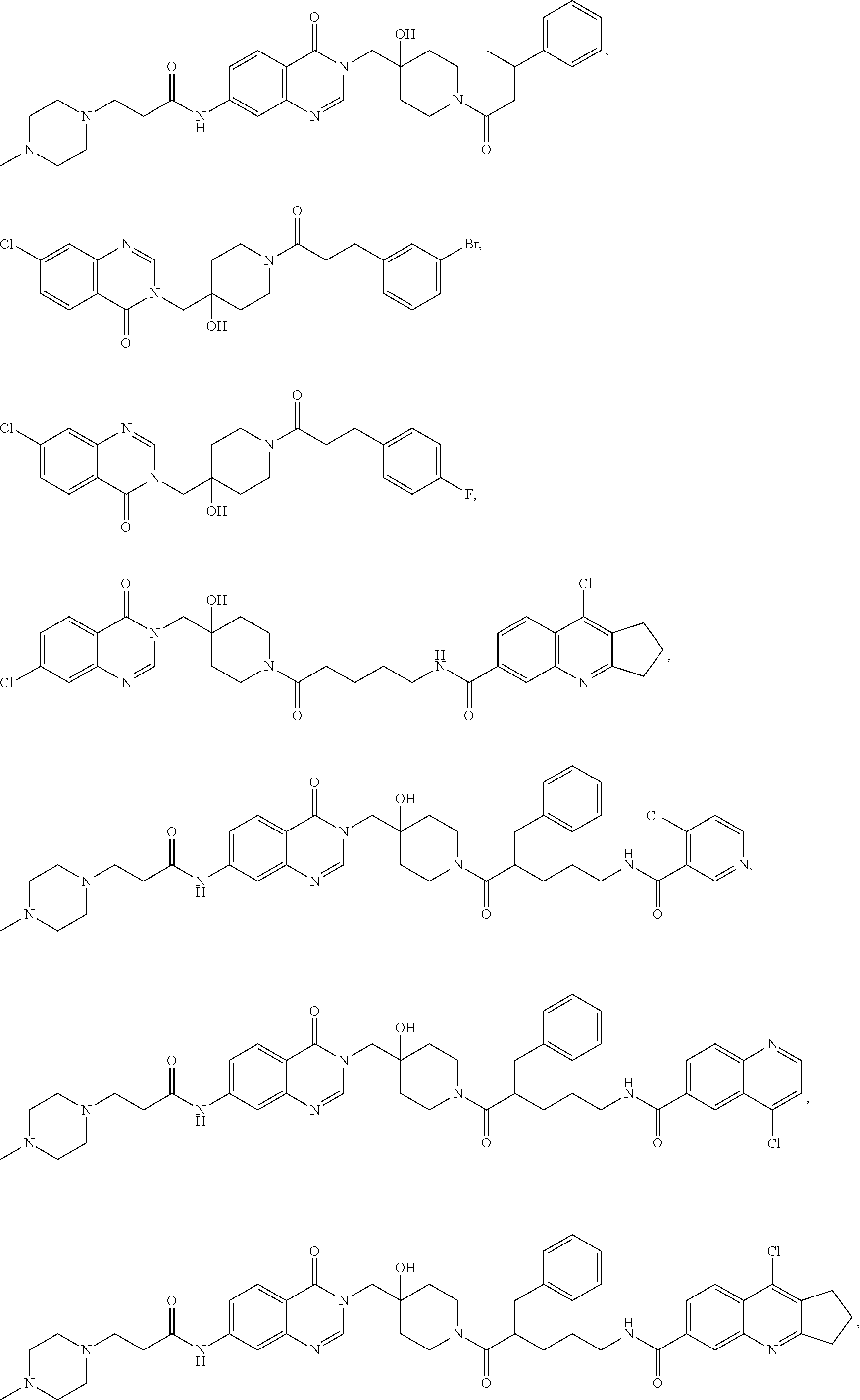

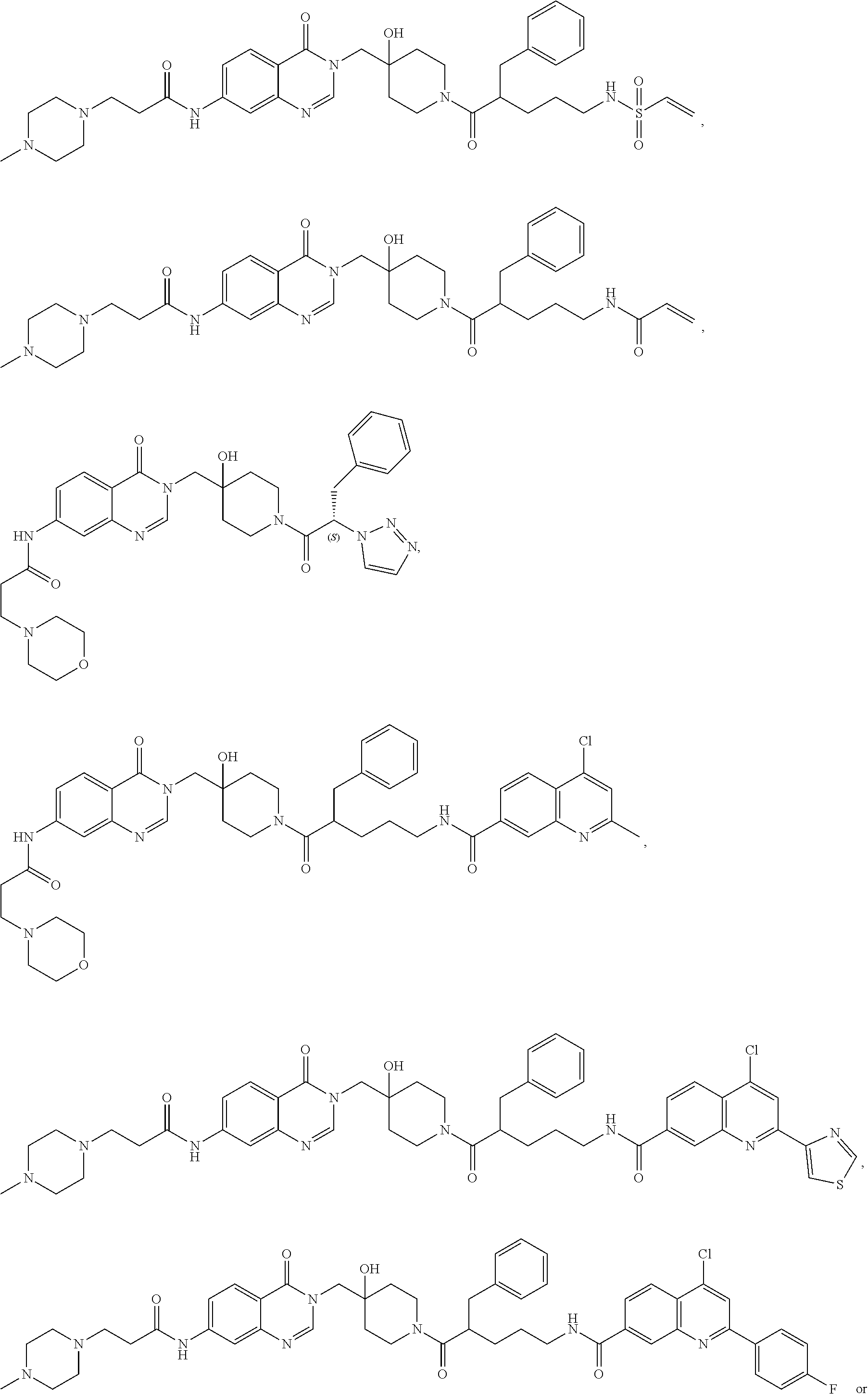

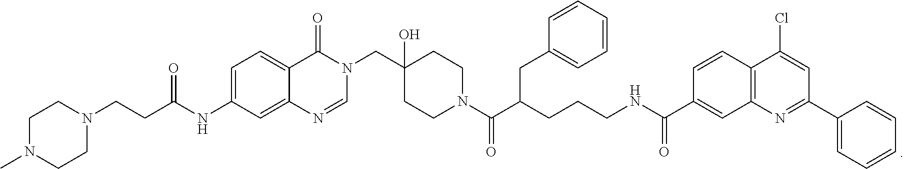 [SMILES: CN1CCN(CCC(=O)Nc2ccc3c(=O)n(CC4(O)CCN(C(=O)C(CCCNC(=O)c5ccc6c(Cl)cc(-c7ccccc7)nc6c5)Cc5ccccc5)CC4)cnc3c2)CC1]

In certain embodiments the compound of Formula (I) is selected from the group consisting of:

| Cmpnd | |
|---|---|
| No. | Compound structure |
| 4 | 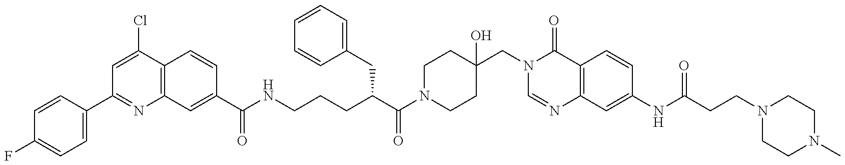 [SMILES: CN1CCN(CCC(=O)Nc2ccc3c(=O)n(CC4(O)CCN(C(=O)[C@H](CCCNC(=O)c5ccc6c(Cl)cc(-c7ccc(F)cc7)nc6c5)Cc5ccccc5)CC4)cnc3c2)CC1] |
| 5 | 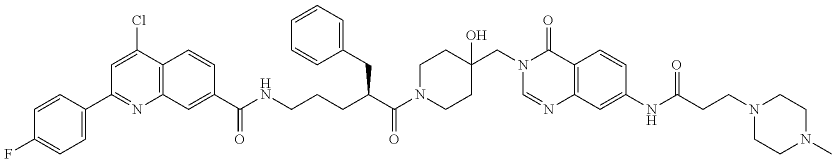 [SMILES: CN1CCN(CCC(=O)Nc2ccc3c(=O)n(CC4(O)CCN(C(=O)[C@@H](CCCNC(=O)c5ccc6c(Cl)cc(-c7ccc(F)cc7)nc6c5)Cc5ccccc5)CC4)cnc3c2)CC1] |
| 6 | 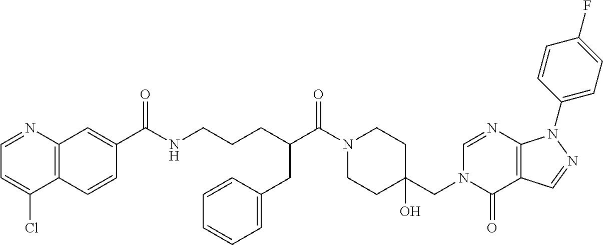 [SMILES: O=C(NCCCC(Cc1ccccc1)C(=O)N1CCC(O)(Cn2cnc3c(cnn3-c3ccc(F)cc3)c2=O)CC1)c1ccc2c(Cl)ccnc2c1] |
| 7 | 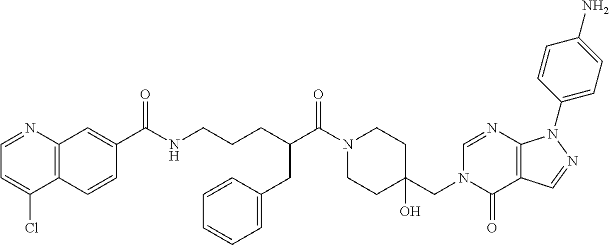 [SMILES: Nc1ccc(-n2ncc3c(=O)n(CC4(O)CCN(C(=O)C(CCCNC(=O)c5ccc6c(Cl)ccnc6c5)Cc5ccccc5)CC4)cnc32)cc1] |
| 8 | 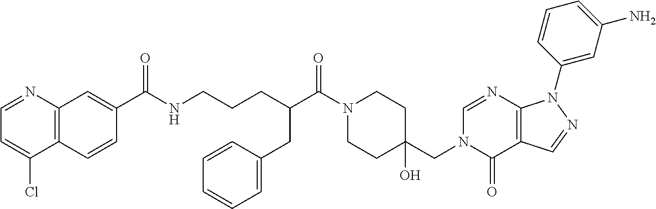 [SMILES: Nc1cccc(-n2ncc3c(=O)n(CC4(O)CCN(C(=O)C(CCCNC(=O)c5ccc6c(Cl)ccnc6c5)Cc5ccccc5)CC4)cnc32)c1] |
| 9 | 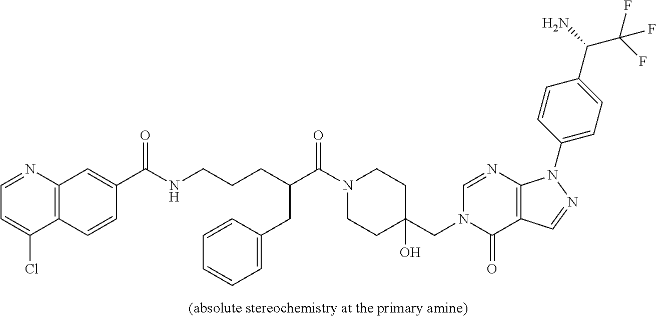 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F] |
| 10 | 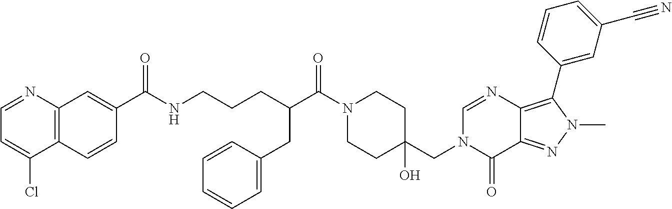 [SMILES: Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1] |
| 11 | 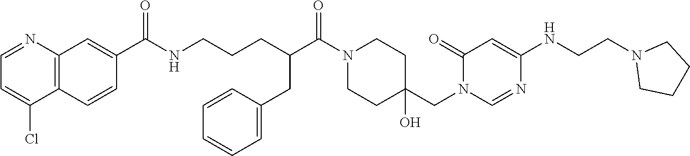 [SMILES: O=C(NCCCC(Cc1ccccc1)C(=O)N1CCC(O)(Cn2cnc(NCCN3CCCC3)cc2=O)CC1)c1ccc2c(Cl)ccnc2c1] |
| 12 | 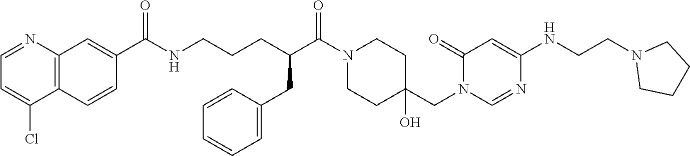 [SMILES: O=C(NCCC[C@H](Cc1ccccc1)C(=O)N1CCC(O)(Cn2cnc(NCCN3CCCC3)cc2=O)CC1)c1ccc2c(Cl)ccnc2c1] |
| 13 | 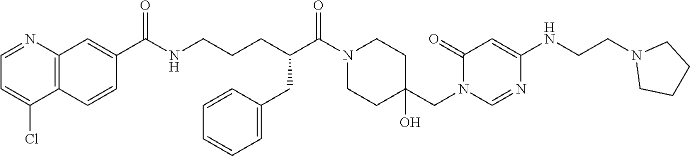 [SMILES: O=C(NCCC[C@@H](Cc1ccccc1)C(=O)N1CCC(O)(Cn2cnc(NCCN3CCCC3)cc2=O)CC1)c1ccc2c(Cl)ccnc2c1] |
| 14 | 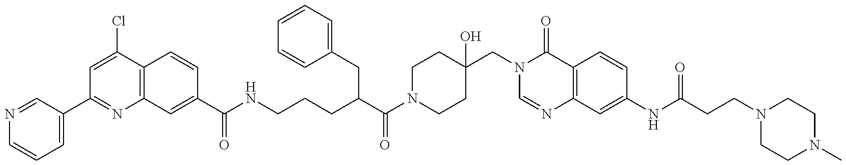 [SMILES: CN1CCN(CCC(=O)Nc2ccc3c(=O)n(CC4(O)CCN(C(=O)C(CCCNC(=O)c5ccc6c(Cl)cc(-c7cccnc7)nc6c5)Cc5ccccc5)CC4)cnc3c2)CC1] |
| 15 | 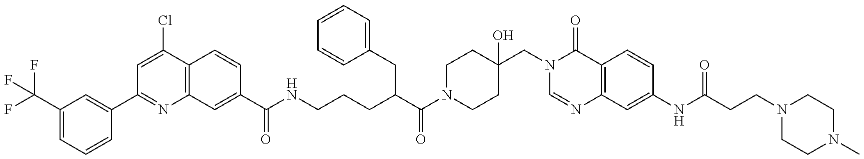 [SMILES: CN1CCN(CCC(=O)Nc2ccc3c(=O)n(CC4(O)CCN(C(=O)C(CCCNC(=O)c5ccc6c(Cl)cc(-c7cccc(C(F)(F)F)c7)nc6c5)Cc5ccccc5)CC4)cnc3c2)CC1] |
| 16 | 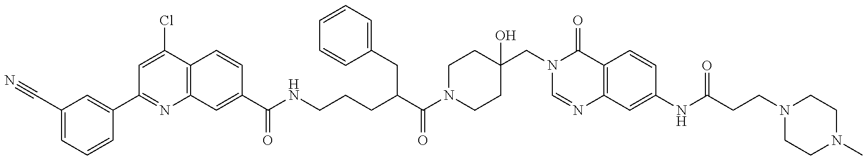 [SMILES: CN1CCN(CCC(=O)Nc2ccc3c(=O)n(CC4(O)CCN(C(=O)C(CCCNC(=O)c5ccc6c(Cl)cc(-c7cccc(C#N)c7)nc6c5)Cc5ccccc5)CC4)cnc3c2)CC1] |
| 17 | 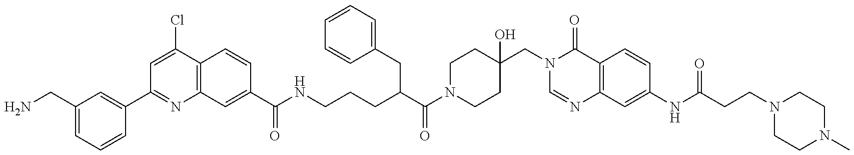 [SMILES: CN1CCN(CCC(=O)Nc2ccc3c(=O)n(CC4(O)CCN(C(=O)C(CCCNC(=O)c5ccc6c(Cl)cc(-c7cccc(CN)c7)nc6c5)Cc5ccccc5)CC4)cnc3c2)CC1] |
| 18 | 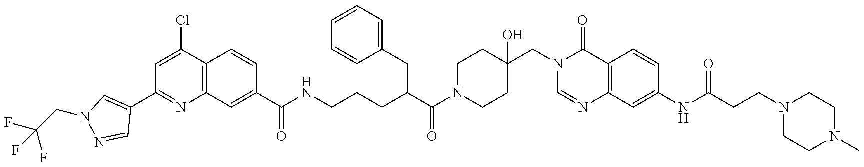 [SMILES: CN1CCN(CCC(=O)Nc2ccc3c(=O)n(CC4(O)CCN(C(=O)C(CCCNC(=O)c5ccc6c(Cl)cc(-c7cnn(CC(F)(F)F)c7)nc6c5)Cc5ccccc5)CC4)cnc3c2)CC1] |
| 19 | 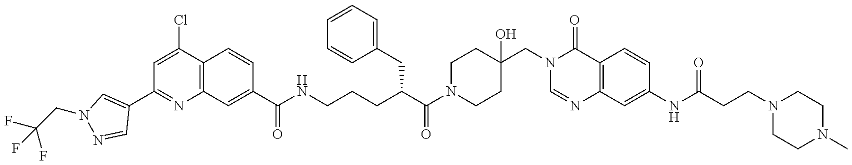 [SMILES: CN1CCN(CCC(=O)Nc2ccc3c(=O)n(CC4(O)CCN(C(=O)[C@H](CCCNC(=O)c5ccc6c(Cl)cc(-c7cnn(CC(F)(F)F)c7)nc6c5)Cc5ccccc5)CC4)cnc3c2)CC1] |
| 20 | 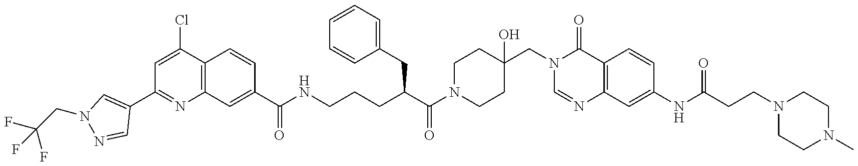 [SMILES: CN1CCN(CCC(=O)Nc2ccc3c(=O)n(CC4(O)CCN(C(=O)[C@@H](CCCNC(=O)c5ccc6c(Cl)cc(-c7cnn(CC(F)(F)F)c7)nc6c5)Cc5ccccc5)CC4)cnc3c2)CC1] |
| 21 | 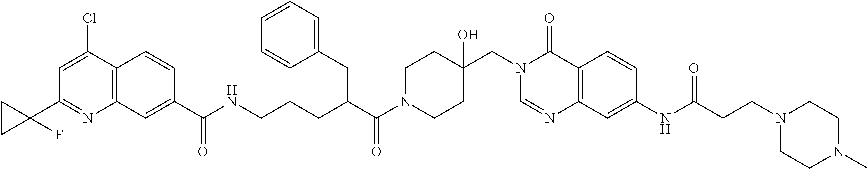 [SMILES: CN1CCN(CCC(=O)Nc2ccc3c(=O)n(CC4(O)CCN(C(=O)C(CCCNC(=O)c5ccc6c(Cl)cc(C7(F)CC7)nc6c5)Cc5ccccc5)CC4)cnc3c2)CC1] |
| 22 | 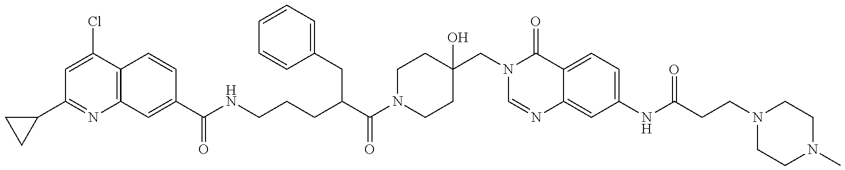 [SMILES: CN1CCN(CCC(=O)Nc2ccc3c(=O)n(CC4(O)CCN(C(=O)C(CCCNC(=O)c5ccc6c(Cl)cc(C7CC7)nc6c5)Cc5ccccc5)CC4)cnc3c2)CC1] |
| 23 | 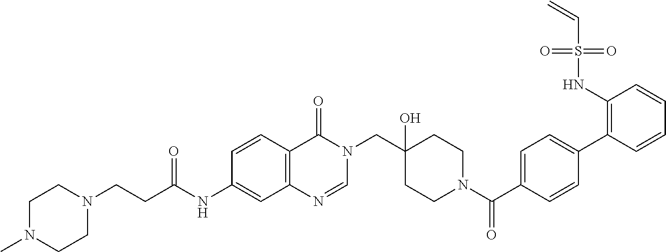 [SMILES: C=CS(=O)(=O)Nc1ccccc1-c1ccc(C(=O)N2CCC(O)(Cn3cnc4cc(NC(=O)CCN5CCN(C)CC5)ccc4c3=O)CC2)cc1] |
| 24 | 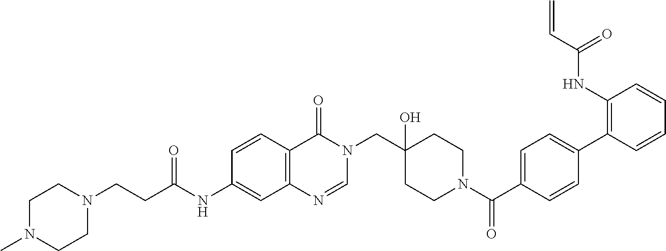 [SMILES: C=CC(=O)Nc1ccccc1-c1ccc(C(=O)N2CCC(O)(Cn3cnc4cc(NC(=O)CCN5CCN(C)CC5)ccc4c3=O)CC2)cc1] |
| 25 | 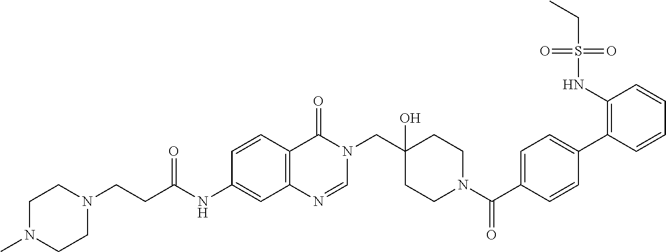 [SMILES: CCS(=O)(=O)Nc1ccccc1-c1ccc(C(=O)N2CCC(O)(Cn3cnc4cc(NC(=O)CCN5CCN(C)CC5)ccc4c3=O)CC2)cc1] |
| 26 | 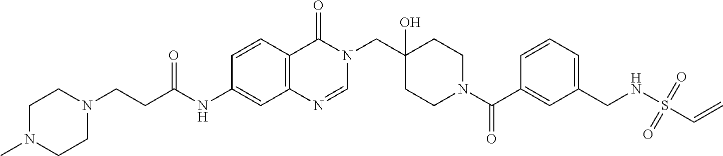 [SMILES: C=CS(=O)(=O)NCc1cccc(C(=O)N2CCC(O)(Cn3cnc4cc(NC(=O)CCN5CCN(C)CC5)ccc4c3=O)CC2)c1] |
| 27 | 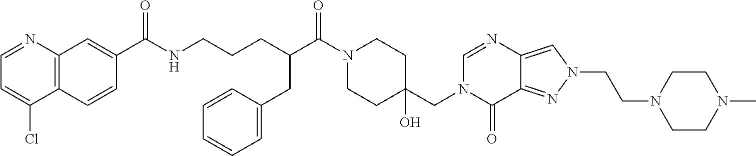 [SMILES: CN1CCN(CCn2cc3ncn(CC4(O)CCN(C(=O)C(CCCNC(=O)c5ccc6c(Cl)ccnc6c5)Cc5ccccc5)CC4)c(=O)c3n2)CC1] |
| 28 | 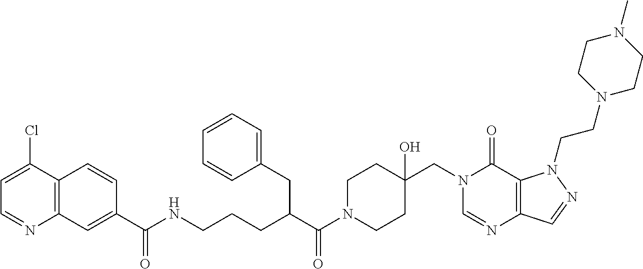 [SMILES: CN1CCN(CCn2ncc3ncn(CC4(O)CCN(C(=O)C(CCCNC(=O)c5ccc6c(Cl)ccnc6c5)Cc5ccccc5)CC4)c(=O)c32)CC1] |
| 29 | 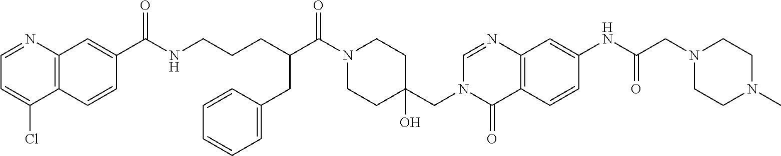 [SMILES: CN1CCN(CC(=O)Nc2ccc3c(=O)n(CC4(O)CCN(C(=O)C(CCCNC(=O)c5ccc6c(Cl)ccnc6c5)Cc5ccccc5)CC4)cnc3c2)CC1] |
| 30 | 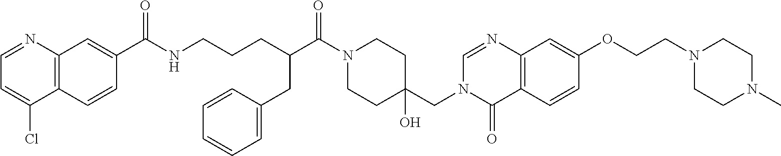 [SMILES: CN1CCN(CCOc2ccc3c(=O)n(CC4(O)CCN(C(=O)C(CCCNC(=O)c5ccc6c(Cl)ccnc6c5)Cc5ccccc5)CC4)cnc3c2)CC1] |
| 31 | 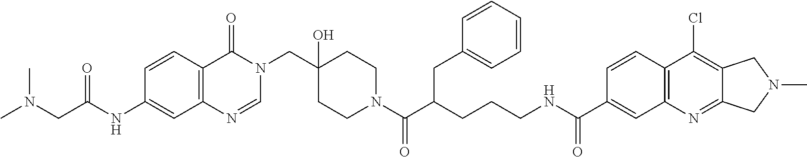 |
| 32 | 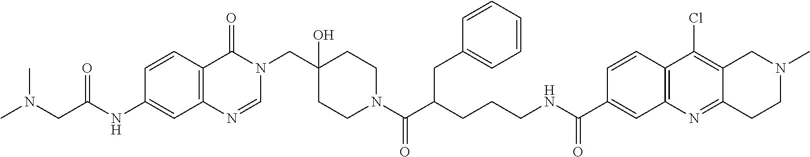 |
| 33 | 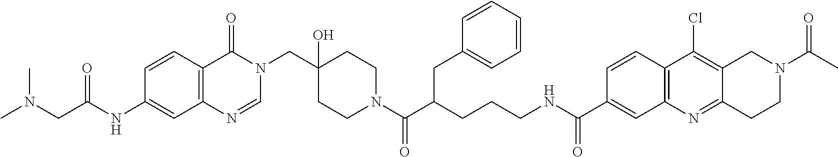 [SMILES: CC(=O)N1CCc2nc3cc(C(=O)NCCCC(Cc4ccccc4)C(=O)N4CCC(O)(Cn5cnc6cc(NC(=O)CN(C)C)ccc6c5=O)CC4)ccc3c(Cl)c2C1] |
| 34 | 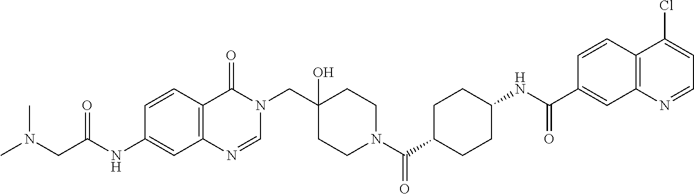 [SMILES: CN(C)CC(=O)Nc1ccc2c(=O)n(CC3(O)CCN(C(=O)[C@H]4CC[C@@H](NC(=O)c5ccc6c(Cl)ccnc6c5)CC4)CC3)cnc2c1] |
| 35 | 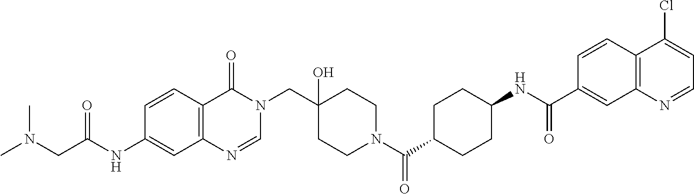 [SMILES: CN(C)CC(=O)Nc1ccc2c(=O)n(CC3(O)CCN(C(=O)[C@H]4CC[C@H](NC(=O)c5ccc6c(Cl)ccnc6c5)CC4)CC3)cnc2c1] |
| 36 | 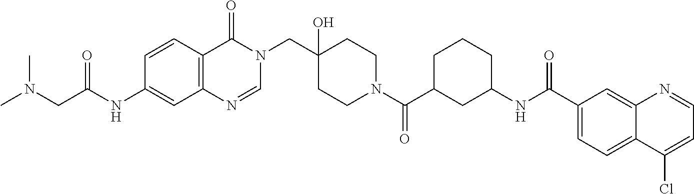 [SMILES: CN(C)CC(=O)Nc1ccc2c(=O)n(CC3(O)CCN(C(=O)C4CCCC(NC(=O)c5ccc6c(Cl)ccnc6c5)C4)CC3)cnc2c1] |
| 37 | 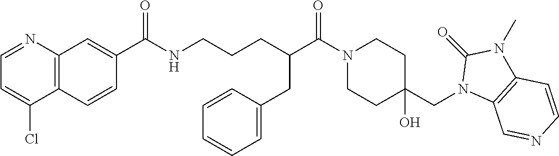 [SMILES: Cn1c(=O)n(CC2(O)CCN(C(=O)C(CCCNC(=O)c3ccc4c(Cl)ccnc4c3)Cc3ccccc3)CC2)c2cnccc21] |
| 38 | 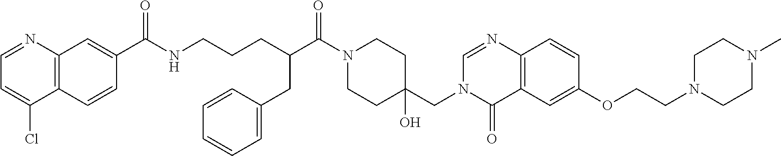 [SMILES: CN1CCN(CCOc2ccc3ncn(CC4(O)CCN(C(=O)C(CCCNC(=O)c5ccc6c(Cl)ccnc6c5)Cc5ccccc5)CC4)c(=O)c3c2)CC1] |
| 39 | 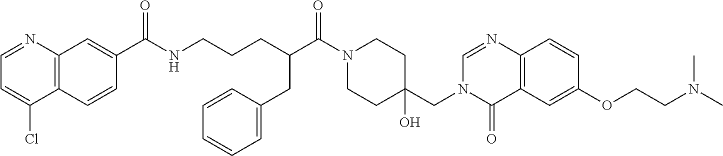 [SMILES: CN(C)CCOc1ccc2ncn(CC3(O)CCN(C(=O)C(CCCNC(=O)c4ccc5c(Cl)ccnc5c4)Cc4ccccc4)CC3)c(=O)c2c1] |
| 40 | 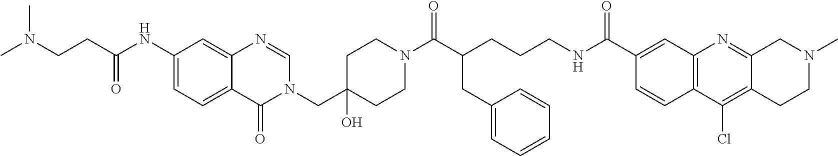 |
| 41 | 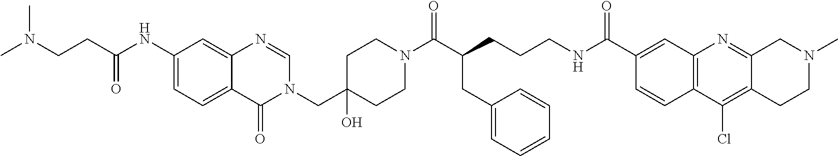 |
| 42 | 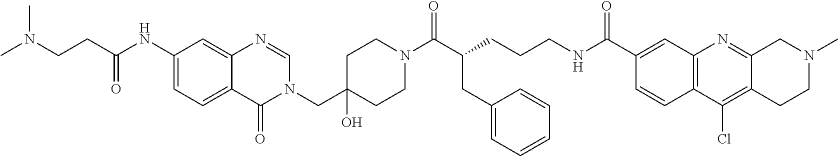 |
| 43 | 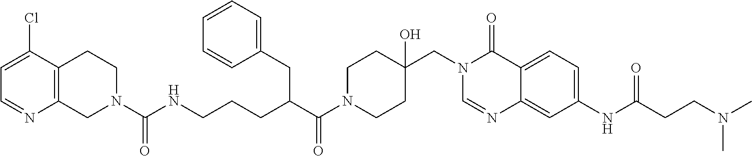 [SMILES: CN(C)CCC(=O)Nc1ccc2c(=O)n(CC3(O)CCN(C(=O)C(CCCNC(=O)N4CCc5c(Cl)ccnc5C4)Cc4ccccc4)CC3)cnc2c1] |
| 44 | 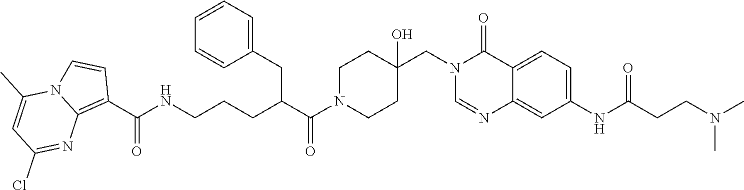 |
| 45 | 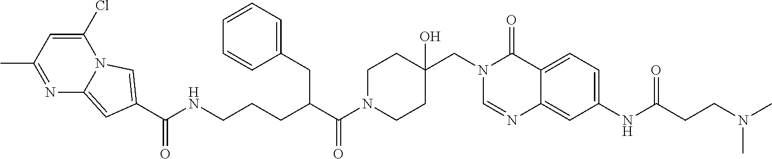 |
| 46 | 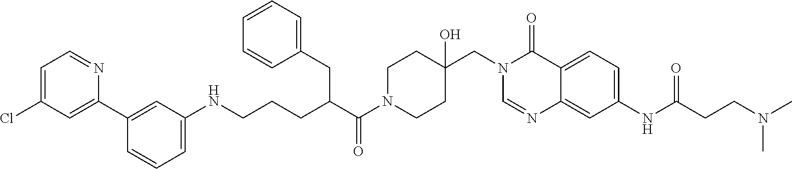 [SMILES: CN(C)CCC(=O)Nc1ccc2c(=O)n(CC3(O)CCN(C(=O)C(CCCNc4cccc(-c5cc(Cl)ccn5)c4)Cc4ccccc4)CC3)cnc2c1] |
| 47 | 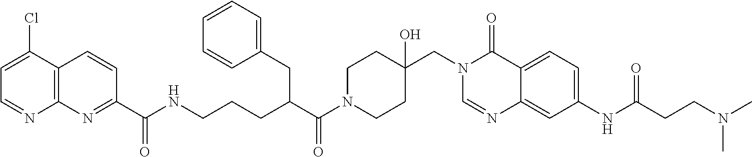 [SMILES: CN(C)CCC(=O)Nc1ccc2c(=O)n(CC3(O)CCN(C(=O)C(CCCNC(=O)c4ccc5c(Cl)ccnc5n4)Cc4ccccc4)CC3)cnc2c1] |
| 48 | 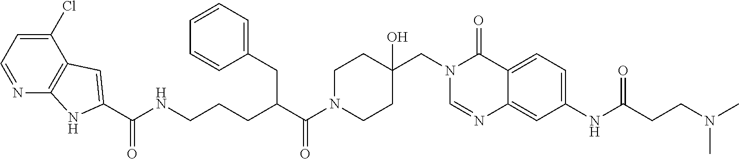 [SMILES: CN(C)CCC(=O)Nc1ccc2c(=O)n(CC3(O)CCN(C(=O)C(CCCNC(=O)c4cc5c(Cl)ccnc5[nH]4)Cc4ccccc4)CC3)cnc2c1] |
| 49 | 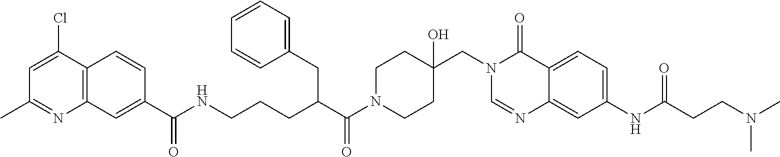 |
| 50 | 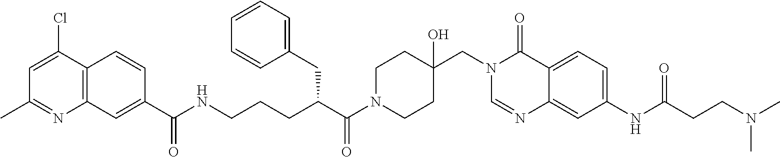 |
| 51 | 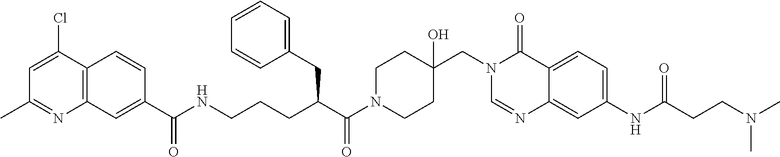 |
| 52 | 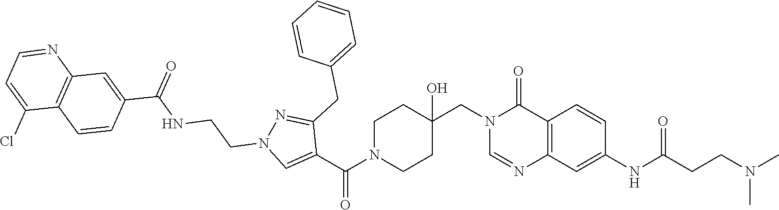 [SMILES: CN(C)CCC(=O)Nc1ccc2c(=O)n(CC3(O)CCN(C(=O)c4cn(CCNC(=O)c5ccc6c(Cl)ccnc6c5)nc4Cc4ccccc4)CC3)cnc2c1] |
| 53 | 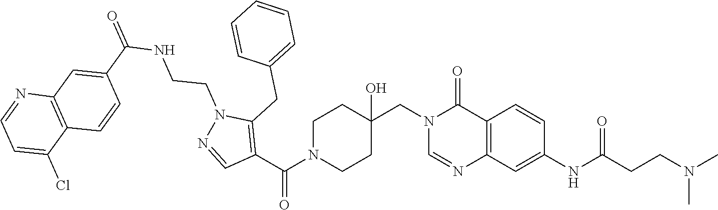 [SMILES: CN(C)CCC(=O)Nc1ccc2c(=O)n(CC3(O)CCN(C(=O)c4cnn(CCNC(=O)c5ccc6c(Cl)ccnc6c5)c4Cc4ccccc4)CC3)cnc2c1] |
| 54 | 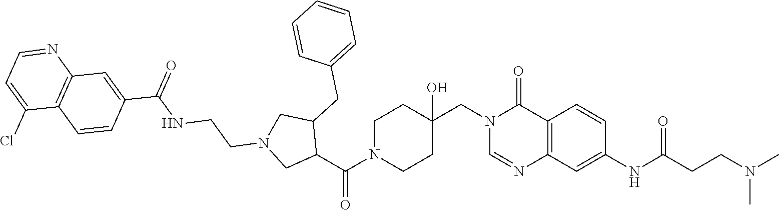 [SMILES: CN(C)CCC(=O)Nc1ccc2c(=O)n(CC3(O)CCN(C(=O)C4CN(CCNC(=O)c5ccc6c(Cl)ccnc6c5)CC4Cc4ccccc4)CC3)cnc2c1] |
| 55 | 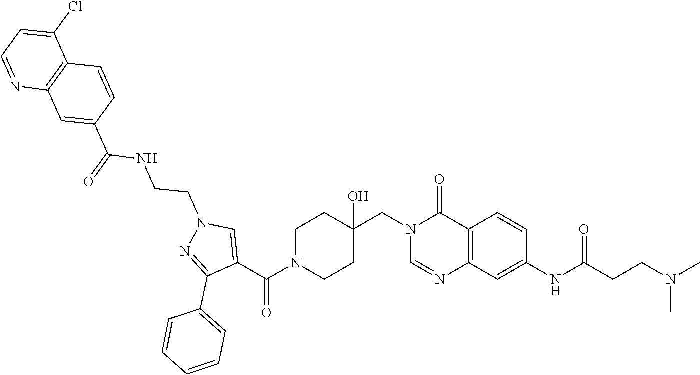 [SMILES: CN(C)CCC(=O)Nc1ccc2c(=O)n(CC3(O)CCN(C(=O)c4cn(CCNC(=O)c5ccc6c(Cl)ccnc6c5)nc4-c4ccccc4)CC3)cnc2c1] |
| 56 | 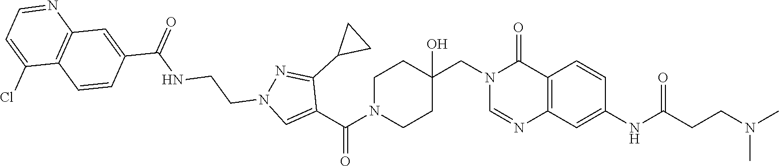 [SMILES: CN(C)CCC(=O)Nc1ccc2c(=O)n(CC3(O)CCN(C(=O)c4cn(CCNC(=O)c5ccc6c(Cl)ccnc6c5)nc4C4CC4)CC3)cnc2c1] |
| 57 | 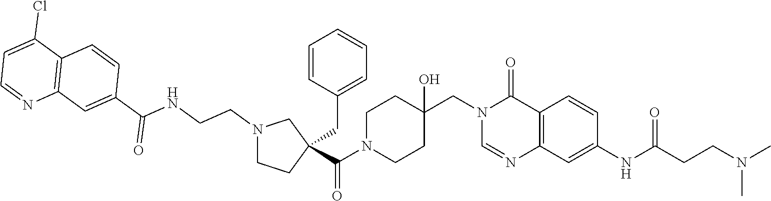 [SMILES: CN(C)CCC(=O)Nc1ccc2c(=O)n(CC3(O)CCN(C(=O)[C@@]4(Cc5ccccc5)CCN(CCNC(=O)c5ccc6c(Cl)ccnc6c5)C4)CC3)cnc2c1] |
| 58 | 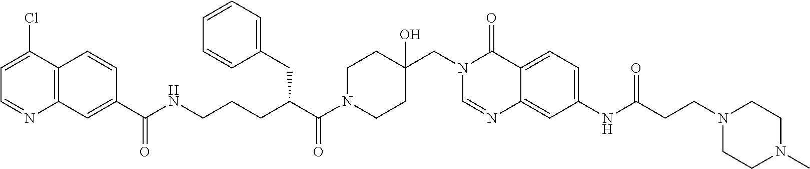 [SMILES: CN1CCN(CCC(=O)Nc2ccc3c(=O)n(CC4(O)CCN(C(=O)[C@H](CCCNC(=O)c5ccc6c(Cl)ccnc6c5)Cc5ccccc5)CC4)cnc3c2)CC1] |
| 59 | 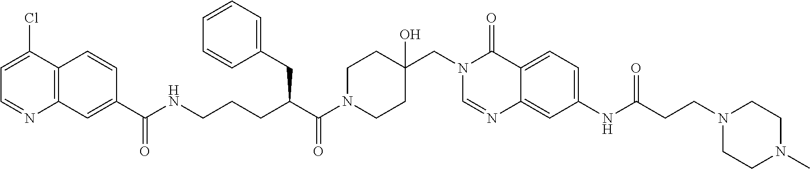 [SMILES: CN1CCN(CCC(=O)Nc2ccc3c(=O)n(CC4(O)CCN(C(=O)[C@@H](CCCNC(=O)c5ccc6c(Cl)ccnc6c5)Cc5ccccc5)CC4)cnc3c2)CC1] |
| 60 | 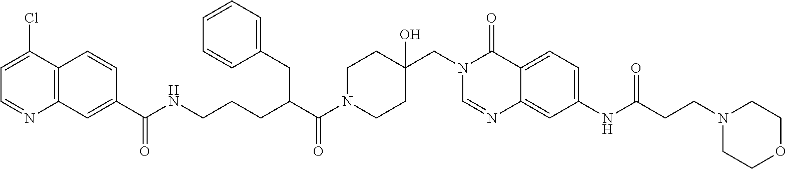 [SMILES: O=C(CCN1CCOCC1)Nc1ccc2c(=O)n(CC3(O)CCN(C(=O)C(CCCNC(=O)c4ccc5c(Cl)ccnc5c4)Cc4ccccc4)CC3)cnc2c1] |
| 61 | 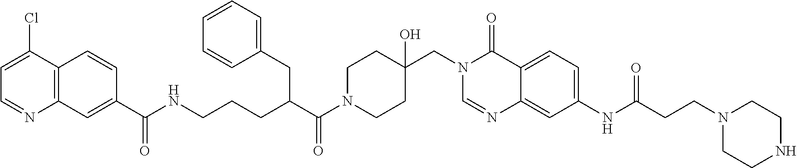 [SMILES: O=C(CCN1CCNCC1)Nc1ccc2c(=O)n(CC3(O)CCN(C(=O)C(CCCNC(=O)c4ccc5c(Cl)ccnc5c4)Cc4ccccc4)CC3)cnc2c1] |
| 62 | 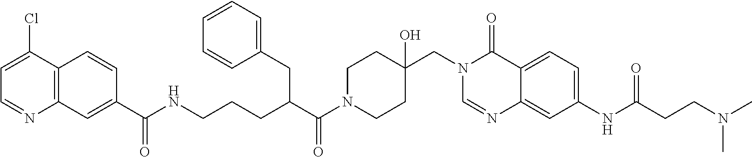 [SMILES: CN(C)CCC(=O)Nc1ccc2c(=O)n(CC3(O)CCN(C(=O)C(CCCNC(=O)c4ccc5c(Cl)ccnc5c4)Cc4ccccc4)CC3)cnc2c1] |
| 63 | 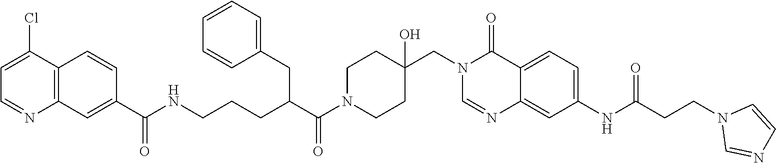 [SMILES: O=C(CCn1ccnc1)Nc1ccc2c(=O)n(CC3(O)CCN(C(=O)C(CCCNC(=O)c4ccc5c(Cl)ccnc5c4)Cc4ccccc4)CC3)cnc2c1] |
| 64 | 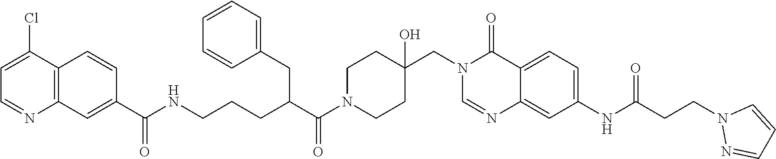 [SMILES: O=C(CCn1cccn1)Nc1ccc2c(=O)n(CC3(O)CCN(C(=O)C(CCCNC(=O)c4ccc5c(Cl)ccnc5c4)Cc4ccccc4)CC3)cnc2c1] |
| 65 | 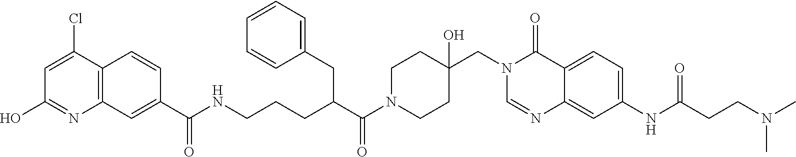 [SMILES: CN(C)CCC(=O)Nc1ccc2c(=O)n(CC3(O)CCN(C(=O)C(CCCNC(=O)c4ccc5c(Cl)cc(O)nc5c4)Cc4ccccc4)CC3)cnc2c1] |
| 66 | 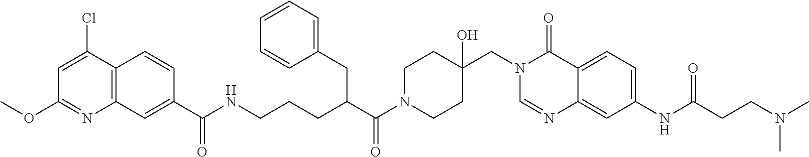 |
| 67 | 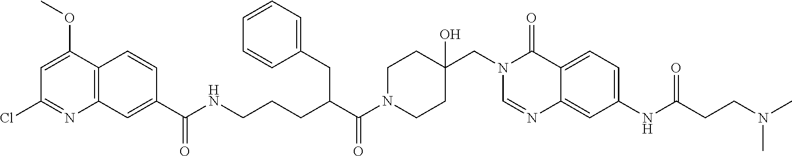 [SMILES: COc1cc(Cl)nc2cc(C(=O)NCCCC(Cc3ccccc3)C(=O)N3CCC(O)(Cn4cnc5cc(NC(=O)CCN(C)C)ccc5c4=O)CC3)ccc12] |
| 68 | 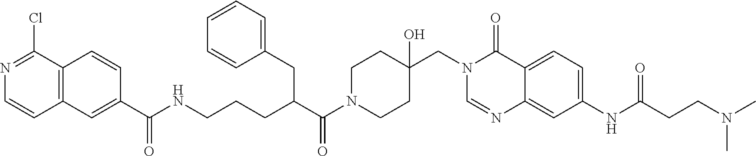 [SMILES: CN(C)CCC(=O)Nc1ccc2c(=O)n(CC3(O)CCN(C(=O)C(CCCNC(=O)c4ccc5c(Cl)nccc5c4)Cc4ccccc4)CC3)cnc2c1] |
| 69 | 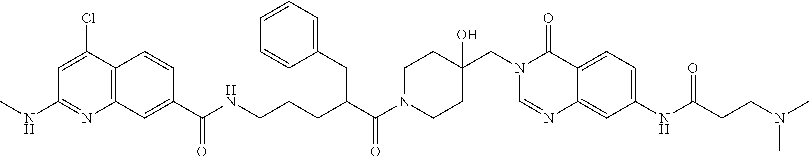 |
| 70 | 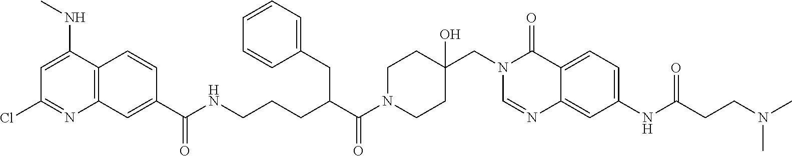 [SMILES: CNc1cc(Cl)nc2cc(C(=O)NCCCC(Cc3ccccc3)C(=O)N3CCC(O)(Cn4cnc5cc(NC(=O)CCN(C)C)ccc5c4=O)CC3)ccc12] |
| 71 | 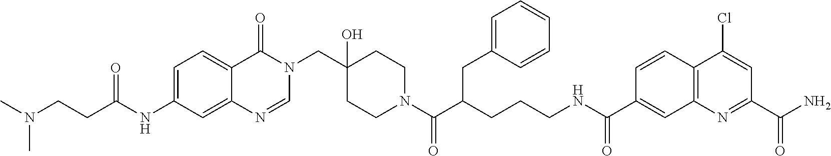 [SMILES: CN(C)CCC(=O)Nc1ccc2c(=O)n(CC3(O)CCN(C(=O)C(CCCNC(=O)c4ccc5c(Cl)cc(C(N)=O)nc5c4)Cc4ccccc4)CC3)cnc2c1] |
| 72 | 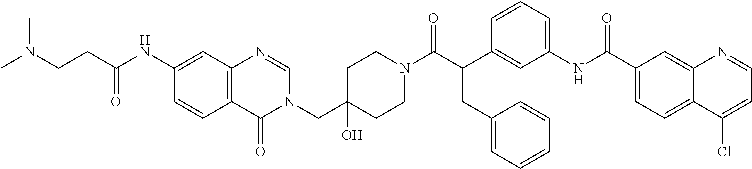 [SMILES: CN(C)CCC(=O)Nc1ccc2c(=O)n(CC3(O)CCN(C(=O)C(Cc4ccccc4)c4cccc(NC(=O)c5ccc6c(Cl)ccnc6c5)c4)CC3)cnc2c1] |
| 73 | 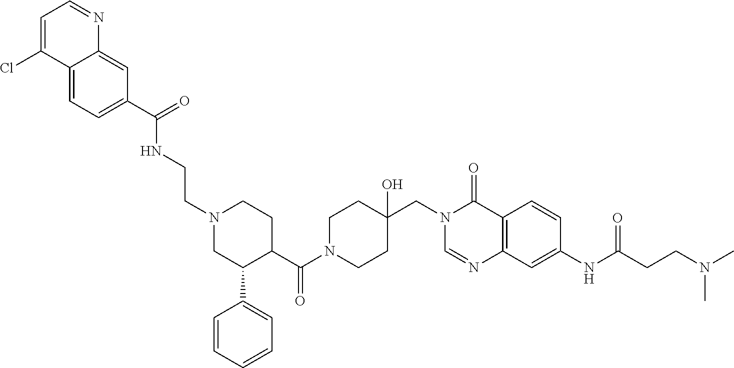 [SMILES: CN(C)CCC(=O)Nc1ccc2c(=O)n(CC3(O)CCN(C(=O)C4CCN(CCNC(=O)c5ccc6c(Cl)ccnc6c5)C[C@H]4c4ccccc4)CC3)cnc2c1] |
| 74 | 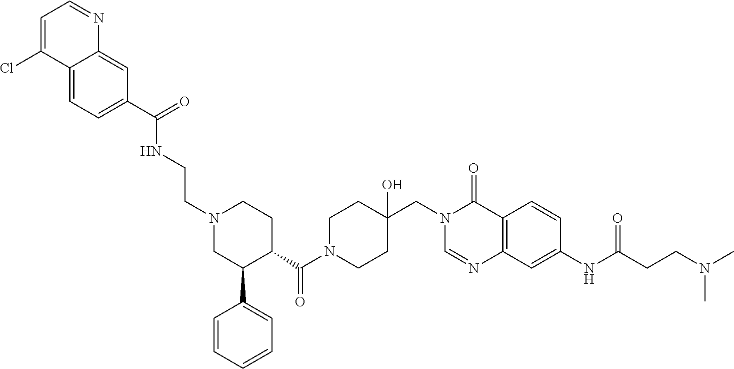 [SMILES: CN(C)CCC(=O)Nc1ccc2c(=O)n(CC3(O)CCN(C(=O)[C@H]4CCN(CCNC(=O)c5ccc6c(Cl)ccnc6c5)C[C@@H]4c4ccccc4)CC3)cnc2c1] |
| 75 | 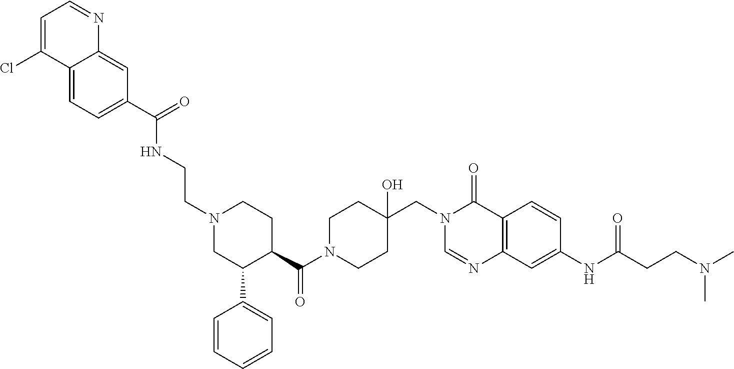 [SMILES: CN(C)CCC(=O)Nc1ccc2c(=O)n(CC3(O)CCN(C(=O)[C@@H]4CCN(CCNC(=O)c5ccc6c(Cl)ccnc6c5)C[C@H]4c4ccccc4)CC3)cnc2c1] |
| 76 | 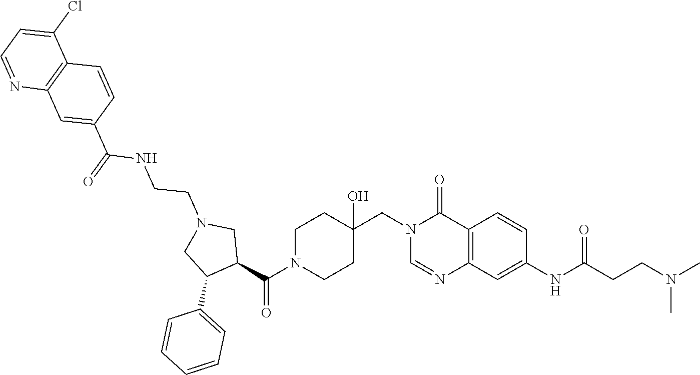 [SMILES: CN(C)CCC(=O)Nc1ccc2c(=O)n(CC3(O)CCN(C(=O)[C@@H]4CN(CCNC(=O)c5ccc6c(Cl)ccnc6c5)C[C@H]4c4ccccc4)CC3)cnc2c1] |
| 77 | 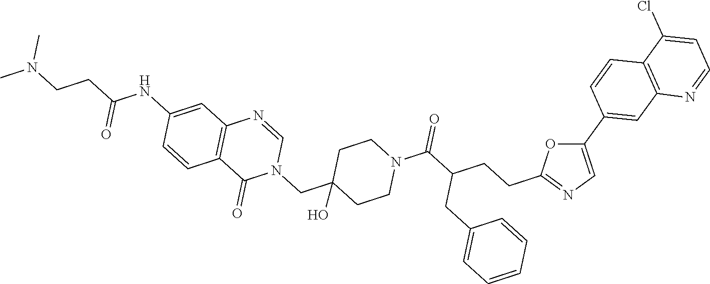 [SMILES: CN(C)CCC(=O)Nc1ccc2c(=O)n(CC3(O)CCN(C(=O)C(CCc4ncc(-c5ccc6c(Cl)ccnc6c5)o4)Cc4ccccc4)CC3)cnc2c1] |
| 78 | 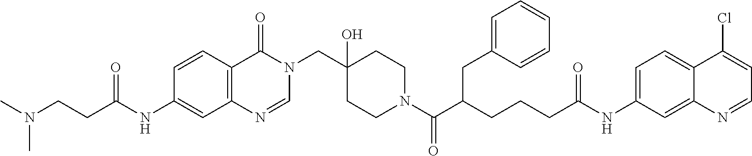 [SMILES: CN(C)CCC(=O)Nc1ccc2c(=O)n(CC3(O)CCN(C(=O)C(CCCC(=O)Nc4ccc5c(Cl)ccnc5c4)Cc4ccccc4)CC3)cnc2c1] |
| 79 | 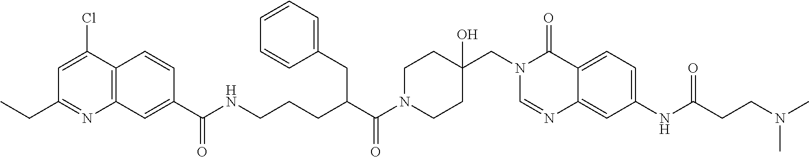 [SMILES: CCc1cc(Cl)c2ccc(C(=O)NCCCC(Cc3ccccc3)C(=O)N3CCC(O)(Cn4cnc5cc(NC(=O)CCN(C)C)ccc5c4=O)CC3)cc2n1] |
| 80 | 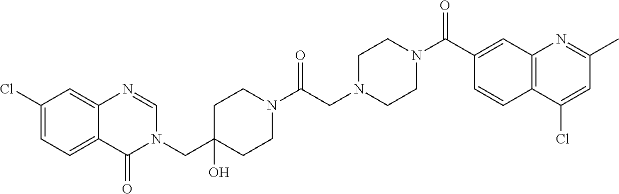 [SMILES: Cc1cc(Cl)c2ccc(C(=O)N3CCN(CC(=O)N4CCC(O)(Cn5cnc6cc(Cl)ccc6c5=O)CC4)CC3)cc2n1] |
| 81 | 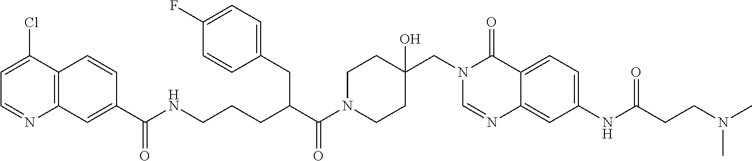 [SMILES: CN(C)CCC(=O)Nc1ccc2c(=O)n(CC3(O)CCN(C(=O)C(CCCNC(=O)c4ccc5c(Cl)ccnc5c4)Cc4ccc(F)cc4)CC3)cnc2c1] |
| 82 | 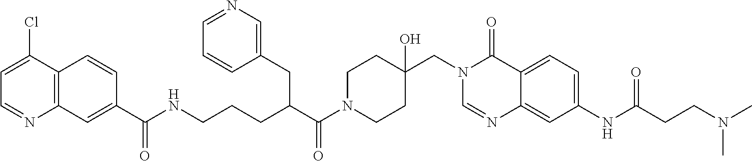 [SMILES: CN(C)CCC(=O)Nc1ccc2c(=O)n(CC3(O)CCN(C(=O)C(CCCNC(=O)c4ccc5c(Cl)ccnc5c4)Cc4cccnc4)CC3)cnc2c1] |
| 83 | 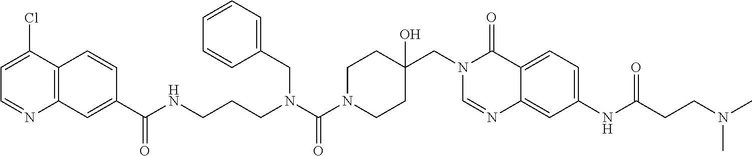 [SMILES: CN(C)CCC(=O)Nc1ccc2c(=O)n(CC3(O)CCN(C(=O)N(CCCNC(=O)c4ccc5c(Cl)ccnc5c4)Cc4ccccc4)CC3)cnc2c1] |
| 84 | 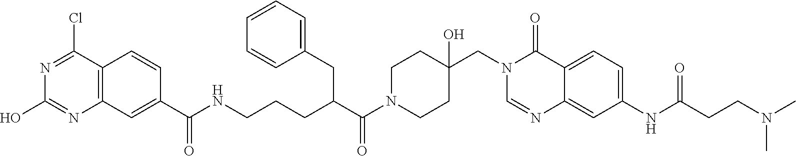 [SMILES: CN(C)CCC(=O)Nc1ccc2c(=O)n(CC3(O)CCN(C(=O)C(CCCNC(=O)c4ccc5c(Cl)nc(O)nc5c4)Cc4ccccc4)CC3)cnc2c1] |
| 85 | 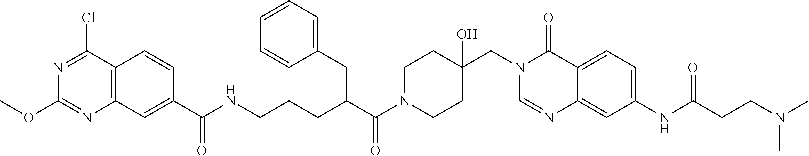 |
| 86 | 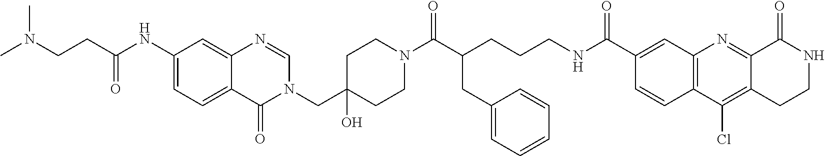 [SMILES: CN(C)CCC(=O)Nc1ccc2c(=O)n(CC3(O)CCN(C(=O)C(CCCNC(=O)c4ccc5c(Cl)c6c(nc5c4)C(=O)NCC6)Cc4ccccc4)CC3)cnc2c1] |
| 87 | 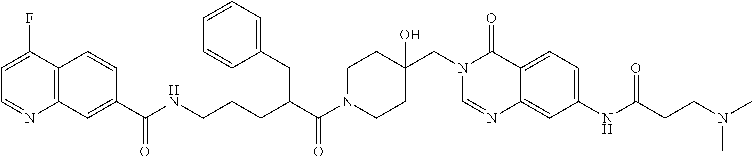 [SMILES: CN(C)CCC(=O)Nc1ccc2c(=O)n(CC3(O)CCN(C(=O)C(CCCNC(=O)c4ccc5c(F)ccnc5c4)Cc4ccccc4)CC3)cnc2c1] |
| 88 | 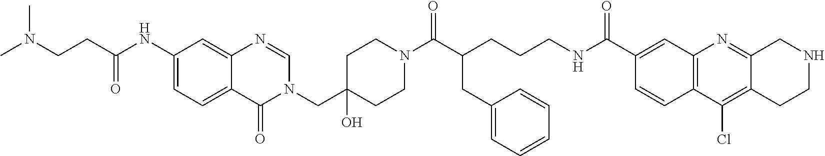 [SMILES: CN(C)CCC(=O)Nc1ccc2c(=O)n(CC3(O)CCN(C(=O)C(CCCNC(=O)c4ccc5c(Cl)c6c(nc5c4)CNCC6)Cc4ccccc4)CC3)cnc2c1] |
| 89 | 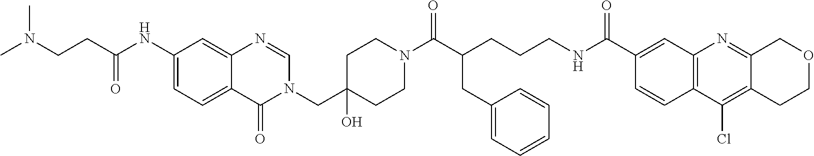 [SMILES: CN(C)CCC(=O)Nc1ccc2c(=O)n(CC3(O)CCN(C(=O)C(CCCNC(=O)c4ccc5c(Cl)c6c(nc5c4)COCC6)Cc4ccccc4)CC3)cnc2c1] |
| 90 | 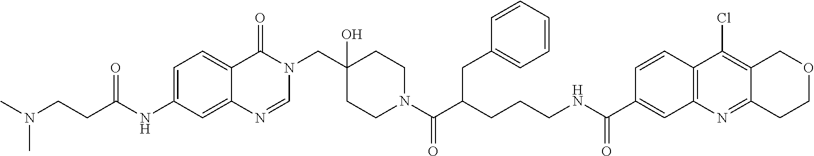 [SMILES: CN(C)CCC(=O)Nc1ccc2c(=O)n(CC3(O)CCN(C(=O)C(CCCNC(=O)c4ccc5c(Cl)c6c(nc5c4)CCOC6)Cc4ccccc4)CC3)cnc2c1] |
| 91 | 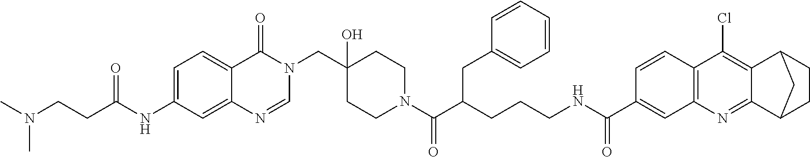 [SMILES: CN(C)CCC(=O)Nc1ccc2c(=O)n(CC3(O)CCN(C(=O)C(CCCNC(=O)c4ccc5c(Cl)c6c(nc5c4)C4CCC6C4)Cc4ccccc4)CC3)cnc2c1] |
| 92 | 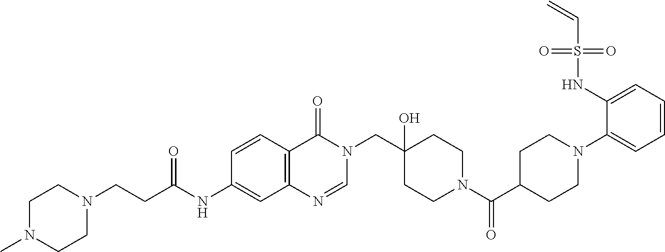 [SMILES: C=CS(=O)(=O)Nc1ccccc1N1CCC(C(=O)N2CCC(O)(Cn3cnc4cc(NC(=O)CCN5CCN(C)CC5)ccc4c3=O)CC2)CC1] |
| 93 | 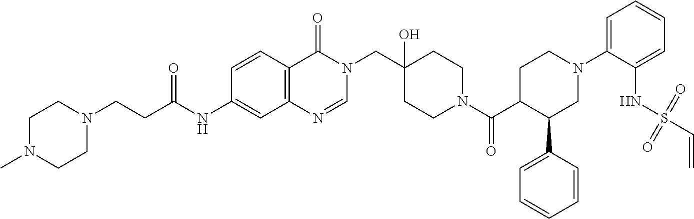 [SMILES: C=CS(=O)(=O)Nc1ccccc1N1CCC(C(=O)N2CCC(O)(Cn3cnc4cc(NC(=O)CCN5CCN(C)CC5)ccc4c3=O)CC2)[C@H](c2ccccc2)C1] |
| 94 | 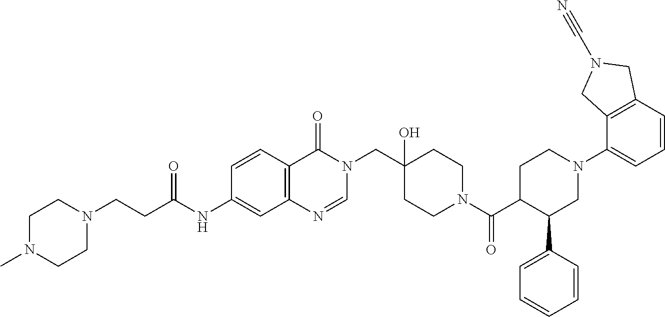 [SMILES: CN1CCN(CCC(=O)Nc2ccc3c(=O)n(CC4(O)CCN(C(=O)C5CCN(c6cccc7c6CN(C#N)C7)C[C@H]5c5ccccc5)CC4)cnc3c2)CC1] |
| 95 | 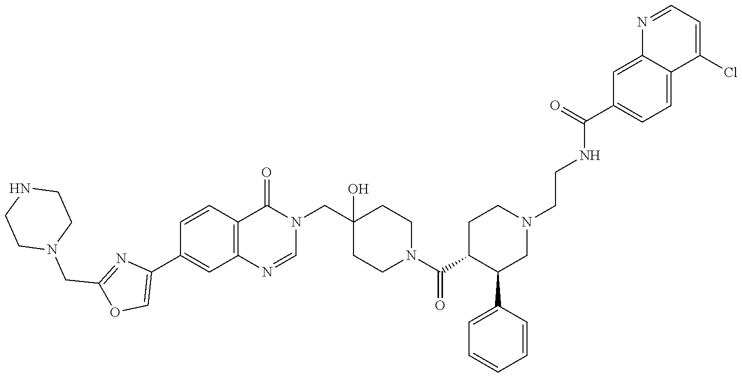 [SMILES: O=C(NCCN1CC[C@@H](C(=O)N2CCC(O)(Cn3cnc4cc(-c5coc(CN6CCNCC6)n5)ccc4c3=O)CC2)[C@H](c2ccccc2)C1)c1ccc2c(Cl)ccnc2c1] |
| 96 | 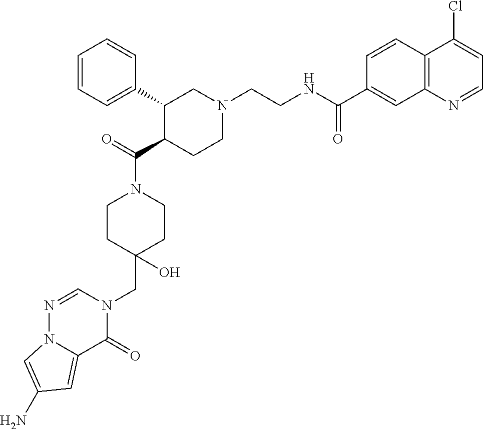 [SMILES: Nc1cc2c(=O)n(CC3(O)CCN(C(=O)[C@@H]4CCN(CCNC(=O)c5ccc6c(Cl)ccnc6c5)C[C@H]4c4ccccc4)CC3)cnn2c1] |
| 97 | 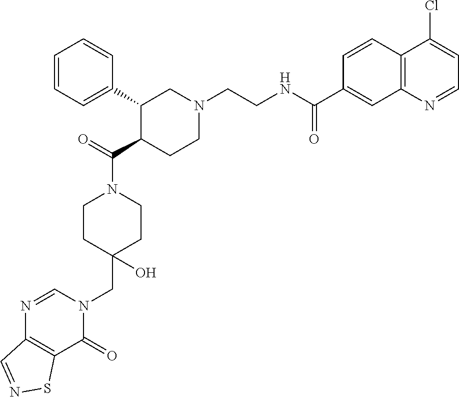 [SMILES: O=C(NCCN1CC[C@@H](C(=O)N2CCC(O)(Cn3cnc4cnsc4c3=O)CC2)[C@H](c2ccccc2)C1)c1ccc2c(Cl)ccnc2c1] |
| 98 | 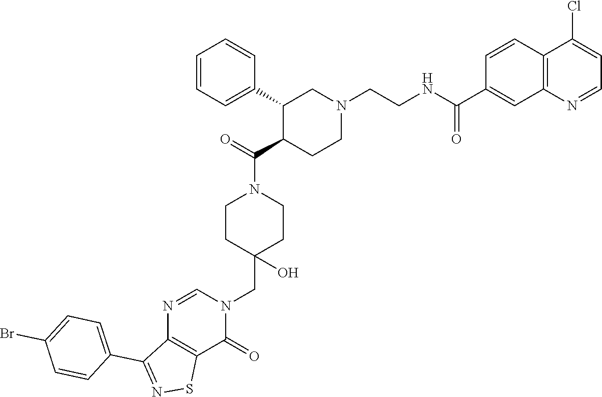 [SMILES: O=C(NCCN1CC[C@@H](C(=O)N2CCC(O)(Cn3cnc4c(-c5ccc(Br)cc5)nsc4c3=O)CC2)[C@H](c2ccccc2)C1)c1ccc2c(Cl)ccnc2c1] |
| 99 | 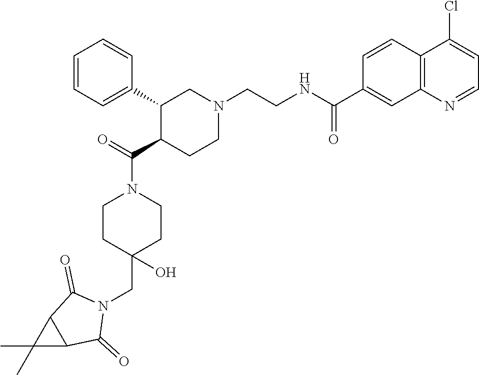 [SMILES: CC1(C)C2C(=O)N(CC3(O)CCN(C(=O)[C@@H]4CCN(CCNC(=O)c5ccc6c(Cl)ccnc6c5)C[C@H]4c4ccccc4)CC3)C(=O)C21] |
| 100 | 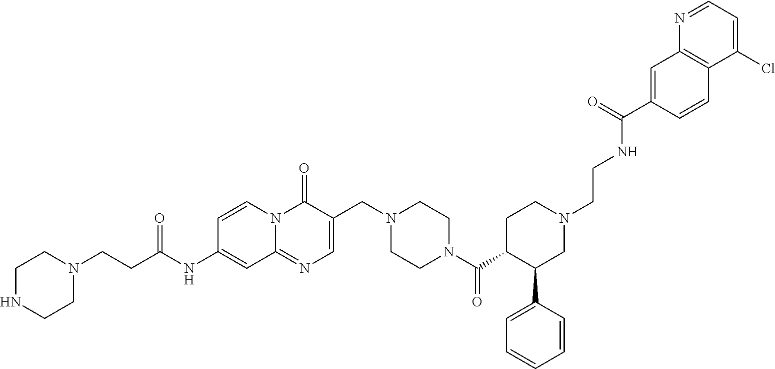 [SMILES: O=C(CCN1CCNCC1)Nc1ccn2c(=O)c(CN3CCN(C(=O)[C@@H]4CCN(CCNC(=O)c5ccc6c(Cl)ccnc6c5)C[C@H]4c4ccccc4)CC3)cnc2c1] |
| 101 | 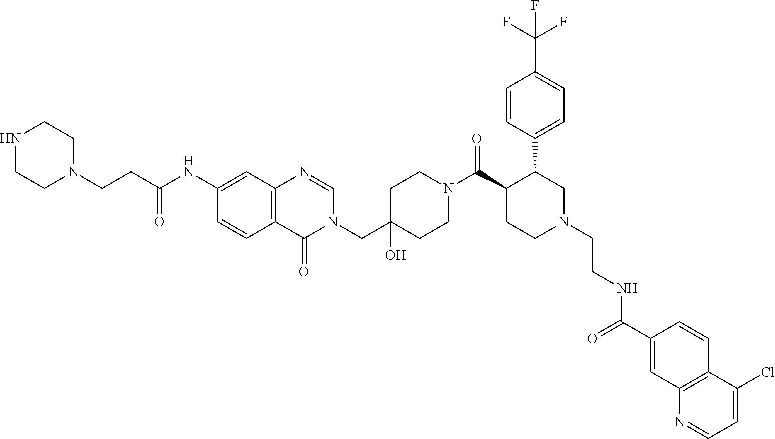 [SMILES: O=C(CCN1CCNCC1)Nc1ccc2c(=O)n(CC3(O)CCN(C(=O)[C@@H]4CCN(CCNC(=O)c5ccc6c(Cl)ccnc6c5)C[C@H]4c4ccc(C(F)(F)F)cc4)CC3)cnc2c1] |
| 102 | 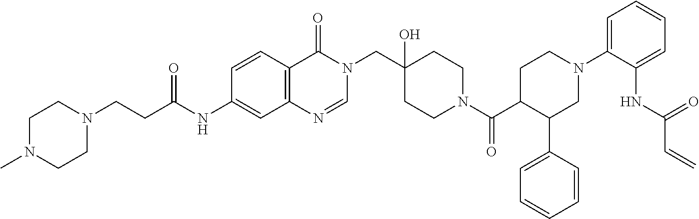 [SMILES: C=CC(=O)Nc1ccccc1N1CCC(C(=O)N2CCC(O)(Cn3cnc4cc(NC(=O)CCN5CCN(C)CC5)ccc4c3=O)CC2)C(c2ccccc2)C1] |
| 103 | 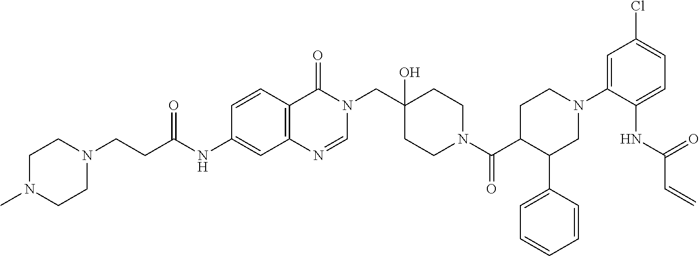 [SMILES: C=CC(=O)Nc1ccc(Cl)cc1N1CCC(C(=O)N2CCC(O)(Cn3cnc4cc(NC(=O)CCN5CCN(C)CC5)ccc4c3=O)CC2)C(c2ccccc2)C1] |
| 104 | 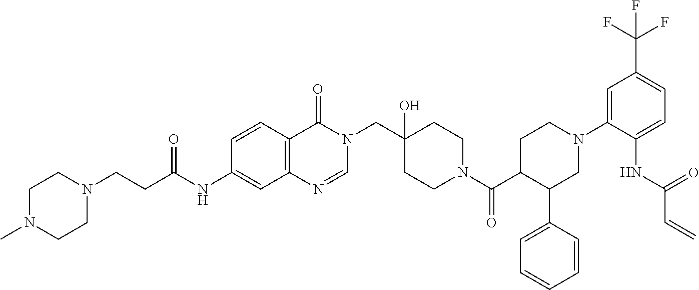 [SMILES: C=CC(=O)Nc1ccc(C(F)(F)F)cc1N1CCC(C(=O)N2CCC(O)(Cn3cnc4cc(NC(=O)CCN5CCN(C)CC5)ccc4c3=O)CC2)C(c2ccccc2)C1] |
| 106 | 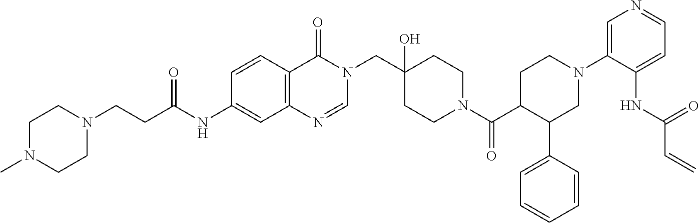 [SMILES: C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1] |
| 107 | 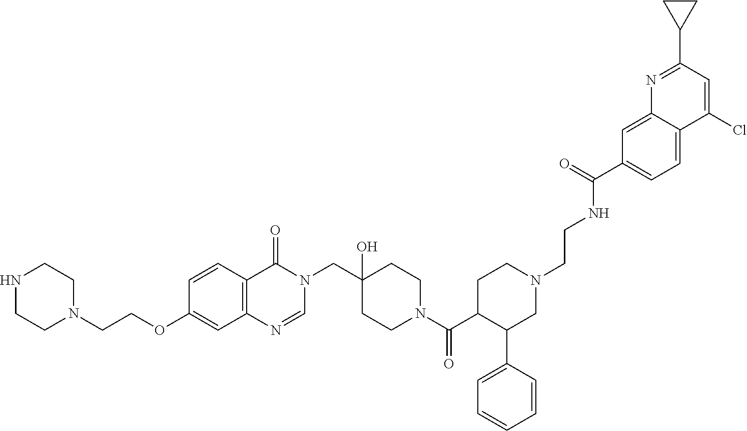 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1] |
| 108 | 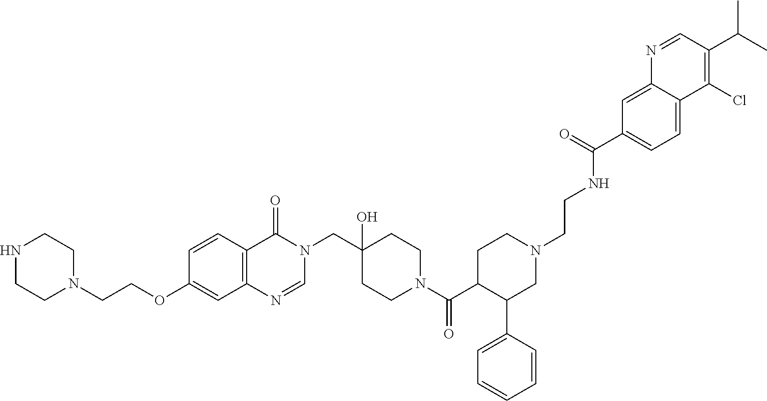 [SMILES: CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl] |
| 109 | 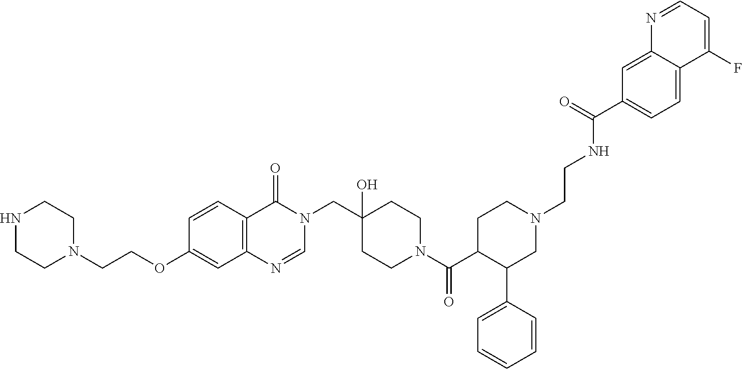 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1] |
| 110 | 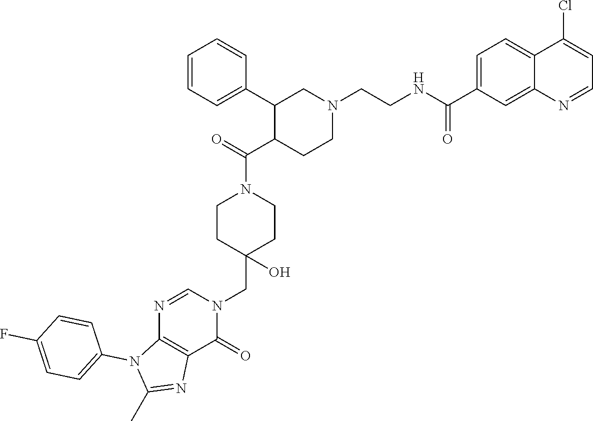 [SMILES: Cc1nc2c(=O)n(CC3(O)CCN(C(=O)C4CCN(CCNC(=O)c5ccc6c(Cl)ccnc6c5)CC4c4ccccc4)CC3)cnc2n1-c1ccc(F)cc1] |
| 111 | 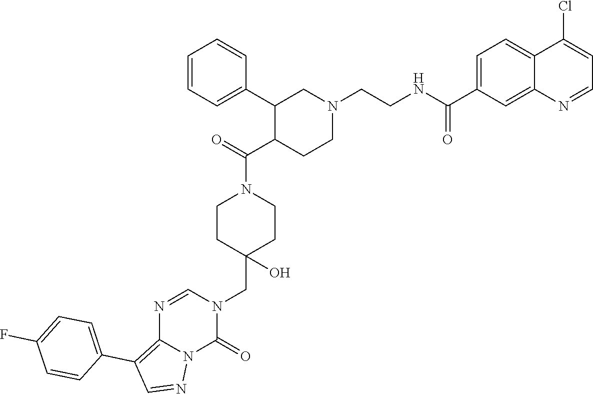 [SMILES: O=C(NCCN1CCC(C(=O)N2CCC(O)(Cn3cnc4c(-c5ccc(F)cc5)cnn4c3=O)CC2)C(c2ccccc2)C1)c1ccc2c(Cl)ccnc2c1] |
| 112 | 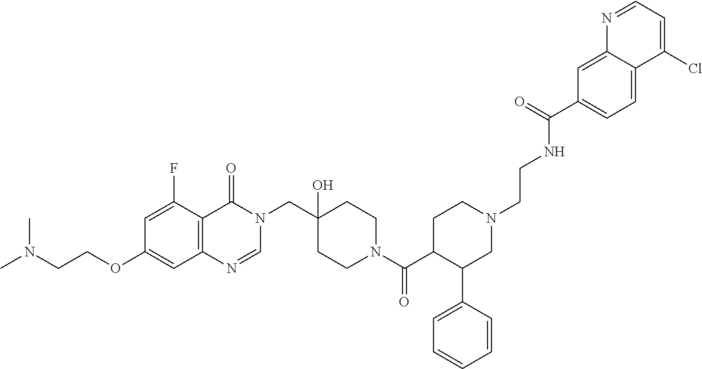 [SMILES: CN(C)CCOc1cc(F)c2c(=O)n(CC3(O)CCN(C(=O)C4CCN(CCNC(=O)c5ccc6c(Cl)ccnc6c5)CC4c4ccccc4)CC3)cnc2c1] |
| 113 | 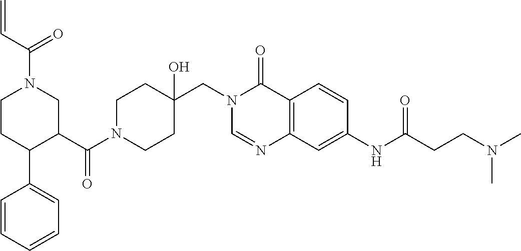 [SMILES: C=CC(=O)N1CCC(c2ccccc2)C(C(=O)N2CCC(O)(Cn3cnc4cc(NC(=O)CCN(C)C)ccc4c3=O)CC2)C1] |
| 114 | 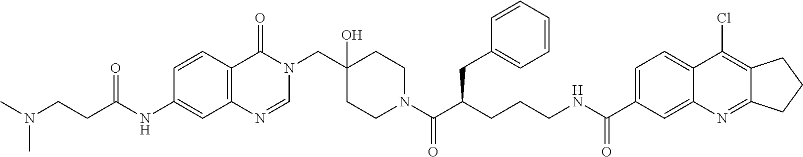 [SMILES: CN(C)CCC(=O)Nc1ccc2c(=O)n(CC3(O)CCN(C(=O)[C@H](CCCNC(=O)c4ccc5c(Cl)c6c(nc5c4)CCC6)Cc4ccccc4)CC3)cnc2c1] |
| 115 | 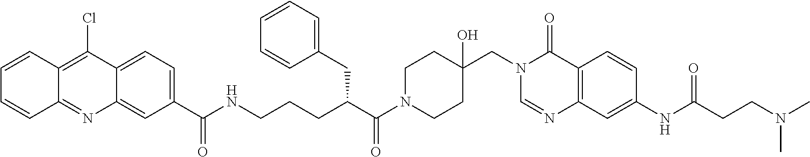 [SMILES: CN(C)CCC(=O)Nc1ccc2c(=O)n(CC3(O)CCN(C(=O)[C@H](CCCNC(=O)c4ccc5c(Cl)c6ccccc6nc5c4)Cc4ccccc4)CC3)cnc2c1] |
| 116 | 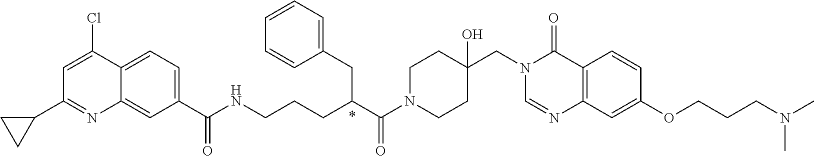 [SMILES: CN(C)CCCOc1ccc2c(=O)n(CC3(O)CCN(C(=O)C(CCCNC(=O)c4ccc5c(Cl)cc(C6CC6)nc5c4)Cc4ccccc4)CC3)cnc2c1] |
| | (absolute stereochemistry not verified; |
| | opposite stereochemistry to next compound at C*) |
| | 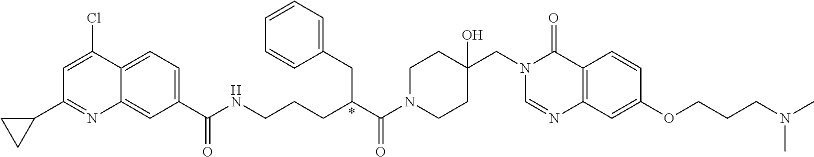 [SMILES: CN(C)CCCOc1ccc2c(=O)n(CC3(O)CCN(C(=O)C(CCCNC(=O)c4ccc5c(Cl)cc(C6CC6)nc5c4)Cc4ccccc4)CC3)cnc2c1] |
| | (absolute stereochemistry not verified; |
| | opposite stereochemistry to 116 at C*) |
| 117 | 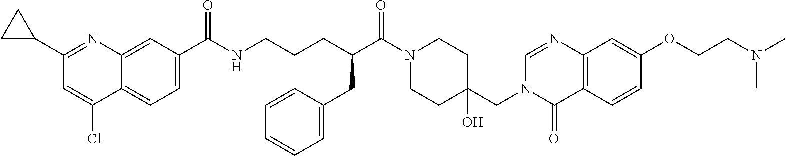 [SMILES: CN(C)CCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1] |
| 118 | 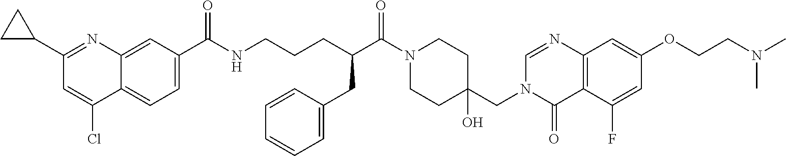 [SMILES: CN(C)CCOc1cc(F)c2c(=O)n(CC3(O)CCN(C(=O)[C@H](CCCNC(=O)c4ccc5c(Cl)cc(C6CC6)nc5c4)Cc4ccccc4)CC3)cnc2c1] |
| 119 | 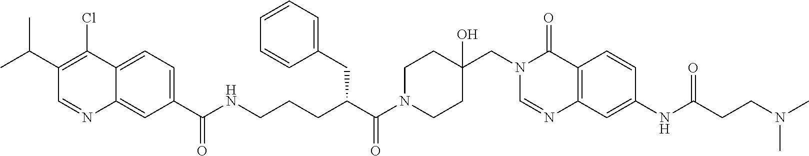 [SMILES: CC(C)c1cnc2cc(C(=O)NCCC[C@H](Cc3ccccc3)C(=O)N3CCC(O)(Cn4cnc5cc(NC(=O)CCN(C)C)ccc5c4=O)CC3)ccc2c1Cl] |
| 120 | 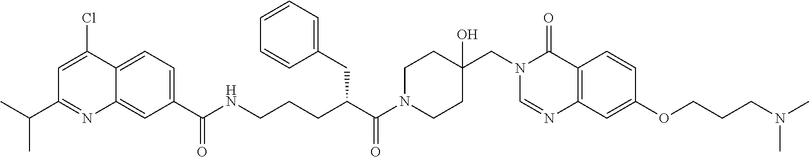 [SMILES: CC(C)c1cc(Cl)c2ccc(C(=O)NCCC[C@H](Cc3ccccc3)C(=O)N3CCC(O)(Cn4cnc5cc(OCCCN(C)C)ccc5c4=O)CC3)cc2n1] |
| 121 | 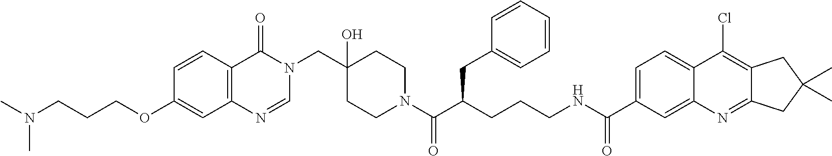 |
| 122 | 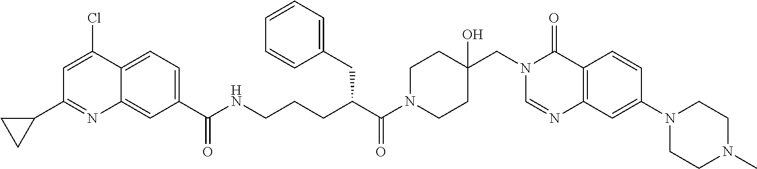 [SMILES: CN1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1] |
| 123 | 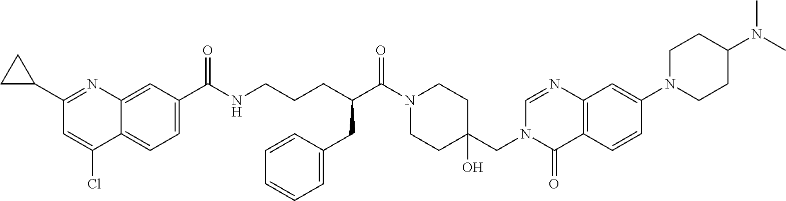 [SMILES: CN(C)C1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1] |
| 124 | 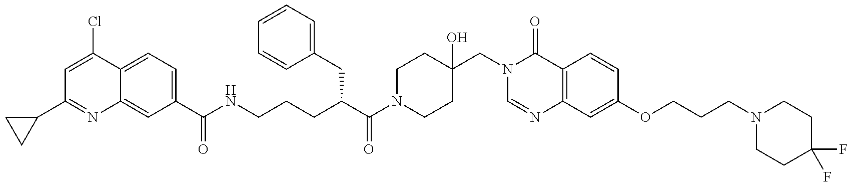 [SMILES: O=C(NCCC[C@H](Cc1ccccc1)C(=O)N1CCC(O)(Cn2cnc3cc(OCCCN4CCC(F)(F)CC4)ccc3c2=O)CC1)c1ccc2c(Cl)cc(C3CC3)nc2c1] |
| 125 | 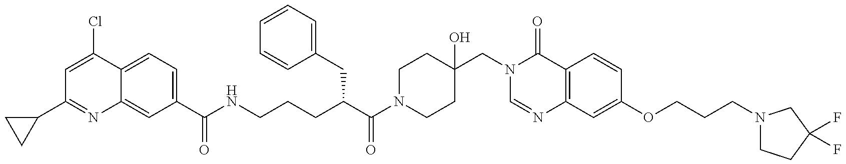 [SMILES: O=C(NCCC[C@H](Cc1ccccc1)C(=O)N1CCC(O)(Cn2cnc3cc(OCCCN4CCC(F)(F)C4)ccc3c2=O)CC1)c1ccc2c(Cl)cc(C3CC3)nc2c1] |
| 126 | 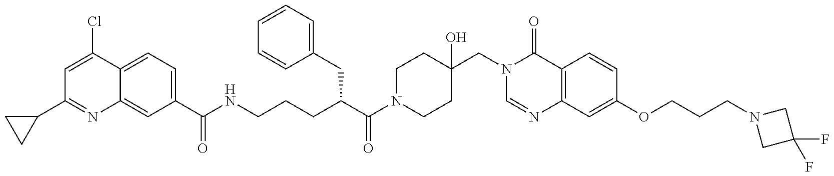 [SMILES: O=C(NCCC[C@H](Cc1ccccc1)C(=O)N1CCC(O)(Cn2cnc3cc(OCCCN4CC(F)(F)C4)ccc3c2=O)CC1)c1ccc2c(Cl)cc(C3CC3)nc2c1] |
| 127 | 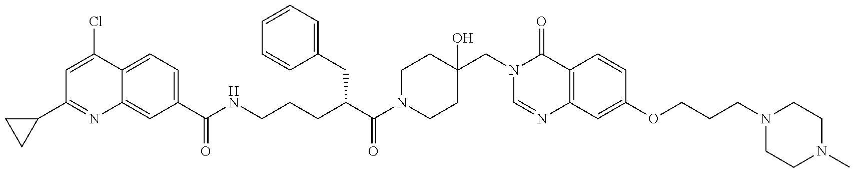 [SMILES: CN1CCN(CCCOc2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1] |
| 128 | 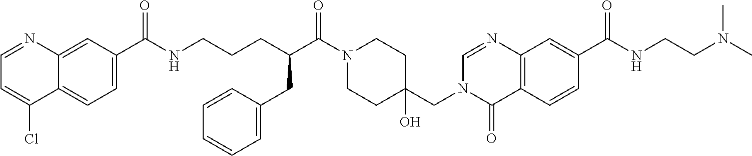 [SMILES: CN(C)CCNC(=O)c1ccc2c(=O)n(CC3(O)CCN(C(=O)[C@H](CCCNC(=O)c4ccc5c(Cl)ccnc5c4)Cc4ccccc4)CC3)cnc2c1] |
| 129 | 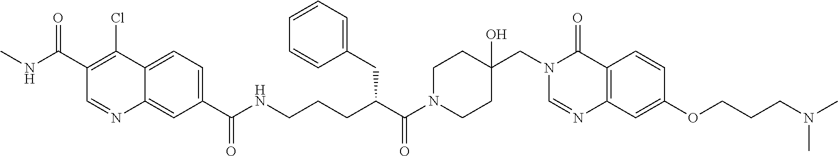 |
| 130 | 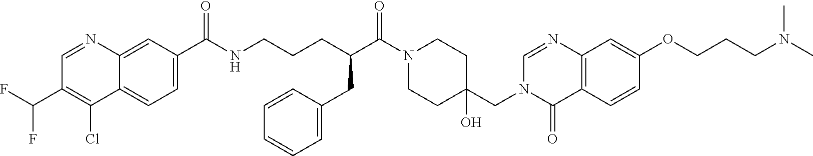 [SMILES: CN(C)CCCOc1ccc2c(=O)n(CC3(O)CCN(C(=O)[C@H](CCCNC(=O)c4ccc5c(Cl)c(C(F)F)cnc5c4)Cc4ccccc4)CC3)cnc2c1] |
| 131 | 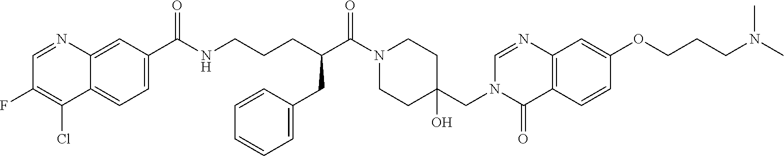 [SMILES: CN(C)CCCOc1ccc2c(=O)n(CC3(O)CCN(C(=O)[C@H](CCCNC(=O)c4ccc5c(Cl)c(F)cnc5c4)Cc4ccccc4)CC3)cnc2c1] |
| 132 | 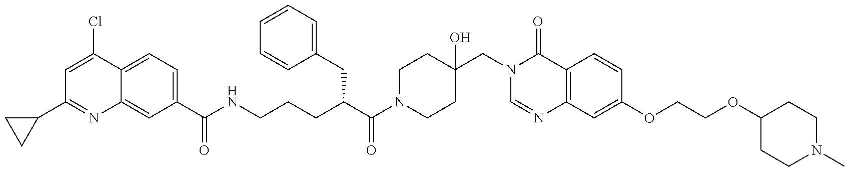 [SMILES: CN1CCC(OCCOc2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1] |
| 133 | 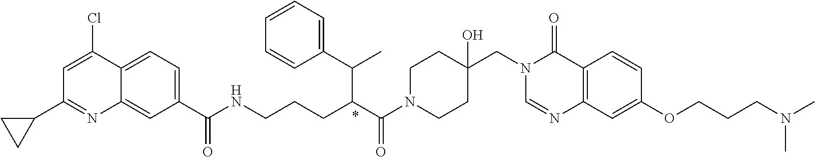 [SMILES: CC(c1ccccc1)C(CCCNC(=O)c1ccc2c(Cl)cc(C3CC3)nc2c1)C(=O)N1CCC(O)(Cn2cnc3cc(OCCCN(C)C)ccc3c2=O)CC1] |
| | (absolute stereochemistry not verified; |
| | opposite stereochmistry to next compound at C*) |
| | 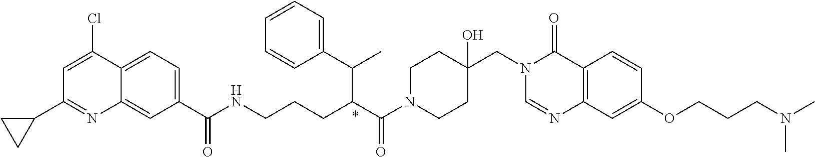 [SMILES: CC(c1ccccc1)C(CCCNC(=O)c1ccc2c(Cl)cc(C3CC3)nc2c1)C(=O)N1CCC(O)(Cn2cnc3cc(OCCCN(C)C)ccc3c2=O)CC1] |
| | (absolute stereochemistry not verified; opposite stereochemistry to 133 at C*) |
| 134 | 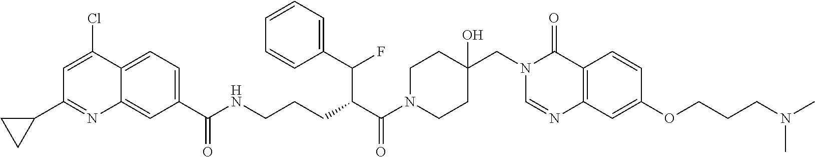 [SMILES: CN(C)CCCOc1ccc2c(=O)n(CC3(O)CCN(C(=O)[C@H](CCCNC(=O)c4ccc5c(Cl)cc(C6CC6)nc5c4)C(F)c4ccccc4)CC3)cnc2c1] |
| 135 | 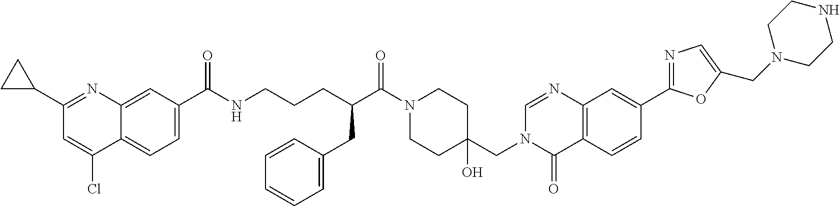 [SMILES: O=C(NCCC[C@H](Cc1ccccc1)C(=O)N1CCC(O)(Cn2cnc3cc(-c4ncc(CN5CCNCC5)o4)ccc3c2=O)CC1)c1ccc2c(Cl)cc(C3CC3)nc2c1] |
| 136 | 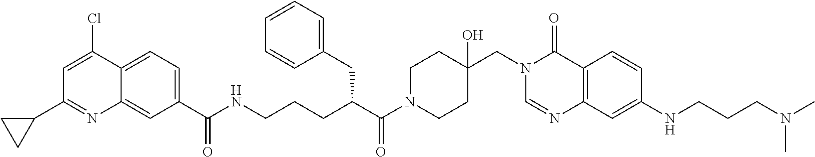 [SMILES: CN(C)CCCNc1ccc2c(=O)n(CC3(O)CCN(C(=O)[C@H](CCCNC(=O)c4ccc5c(Cl)cc(C6CC6)nc5c4)Cc4ccccc4)CC3)cnc2c1] |
| 137 | 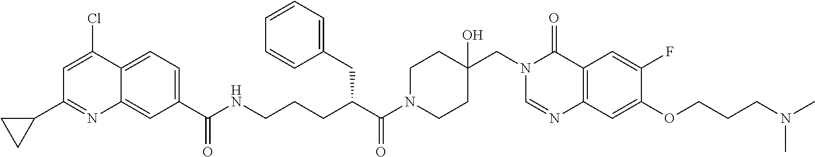 [SMILES: CN(C)CCCOc1cc2ncn(CC3(O)CCN(C(=O)[C@H](CCCNC(=O)c4ccc5c(Cl)cc(C6CC6)nc5c4)Cc4ccccc4)CC3)c(=O)c2cc1F] |
| 138 | 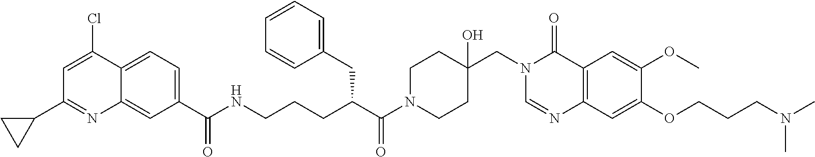 [SMILES: COc1cc2c(=O)n(CC3(O)CCN(C(=O)[C@H](CCCNC(=O)c4ccc5c(Cl)cc(C6CC6)nc5c4)Cc4ccccc4)CC3)cnc2cc1OCCCN(C)C] |
| 139 | 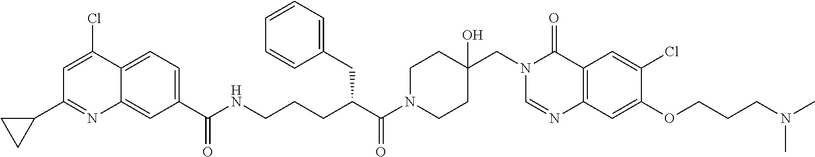 [SMILES: CN(C)CCCOc1cc2ncn(CC3(O)CCN(C(=O)[C@H](CCCNC(=O)c4ccc5c(Cl)cc(C6CC6)nc5c4)Cc4ccccc4)CC3)c(=O)c2cc1Cl] |
| 140 | 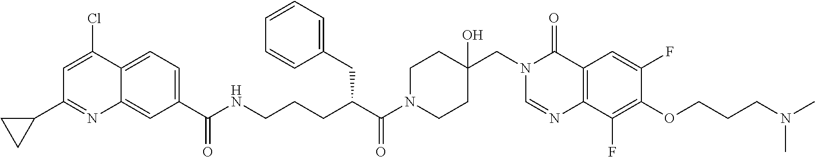 [SMILES: CN(C)CCCOc1c(F)cc2c(=O)n(CC3(O)CCN(C(=O)[C@H](CCCNC(=O)c4ccc5c(Cl)cc(C6CC6)nc5c4)Cc4ccccc4)CC3)cnc2c1F] |
| 141 | 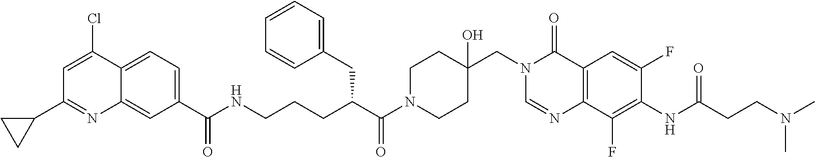 [SMILES: CN(C)CCC(=O)Nc1c(F)cc2c(=O)n(CC3(O)CCN(C(=O)[C@H](CCCNC(=O)c4ccc5c(Cl)cc(C6CC6)nc5c4)Cc4ccccc4)CC3)cnc2c1F] |
| 142 | 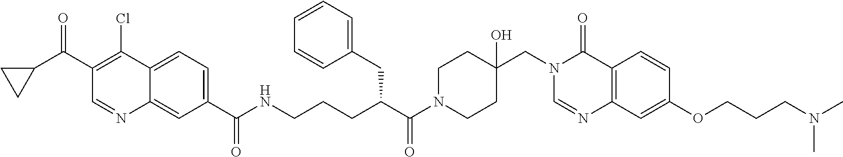 [SMILES: CN(C)CCCOc1ccc2c(=O)n(CC3(O)CCN(C(=O)[C@H](CCCNC(=O)c4ccc5c(Cl)c(C(=O)C6CC6)cnc5c4)Cc4ccccc4)CC3)cnc2c1] |
| 143 | 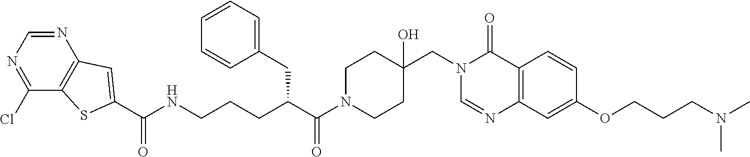 [SMILES: CN(C)CCCOc1ccc2c(=O)n(CC3(O)CCN(C(=O)[C@H](CCCNC(=O)c4cc5ncnc(Cl)c5s4)Cc4ccccc4)CC3)cnc2c1] |
| 144 | 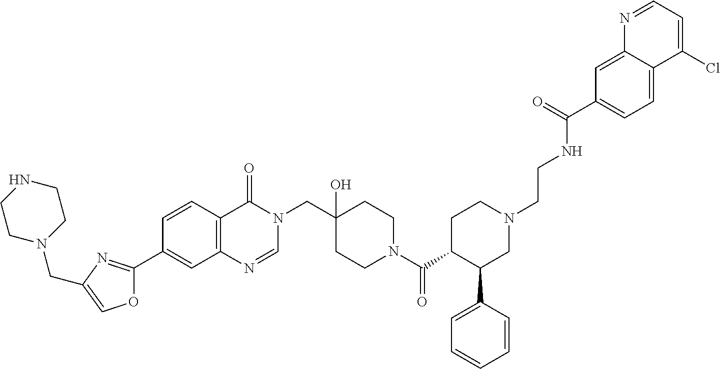 [SMILES: O=C(NCCN1CC[C@@H](C(=O)N2CCC(O)(Cn3cnc4cc(-c5nc(CN6CCNCC6)co5)ccc4c3=O)CC2)[C@H](c2ccccc2)C1)c1ccc2c(Cl)ccnc2c1] |
| | 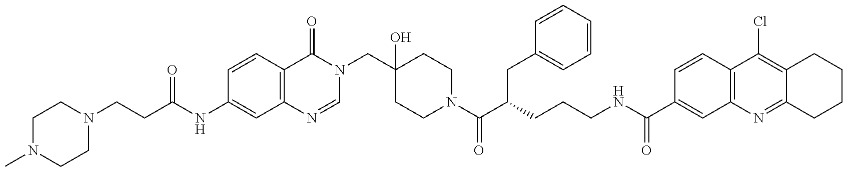 [SMILES: CN1CCN(CCC(=O)Nc2ccc3c(=O)n(CC4(O)CCN(C(=O)[C@@H](CCCNC(=O)c5ccc6c(Cl)c7c(nc6c5)CCCC7)Cc5ccccc5)CC4)cnc3c2)CC1] |
| | *absolute stereochemistry not verified; |
| | opposite stereochemistry as next compound |
| | 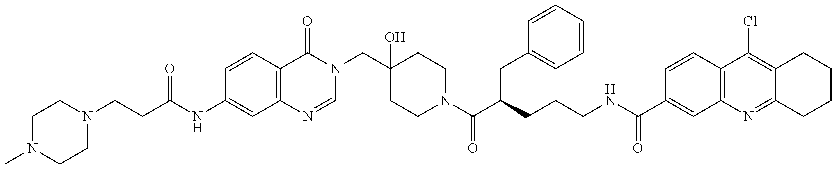 [SMILES: CN1CCN(CCC(=O)Nc2ccc3c(=O)n(CC4(O)CCN(C(=O)[C@H](CCCNC(=O)c5ccc6c(Cl)c7c(nc6c5)CCCC7)Cc5ccccc5)CC4)cnc3c2)CC1] |
| | *absolute sterochemistry not verified; |
| | opposite stereochemistry to previous compound |
| | 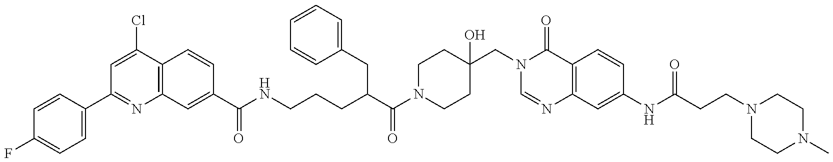 [SMILES: CN1CCN(CCC(=O)Nc2ccc3c(=O)n(CC4(O)CCN(C(=O)C(CCCNC(=O)c5ccc6c(Cl)cc(-c7ccc(F)cc7)nc6c5)Cc5ccccc5)CC4)cnc3c2)CC1] |
| | 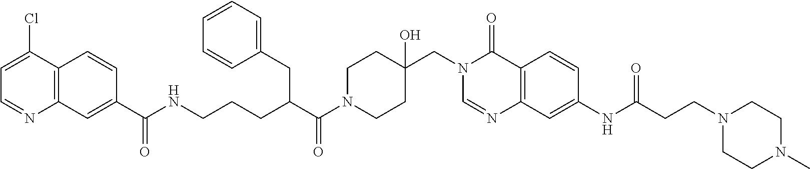 [SMILES: CN1CCN(CCC(=O)Nc2ccc3c(=O)n(CC4(O)CCN(C(=O)C(CCCNC(=O)c5ccc6c(Cl)ccnc6c5)Cc5ccccc5)CC4)cnc3c2)CC1] |
| | 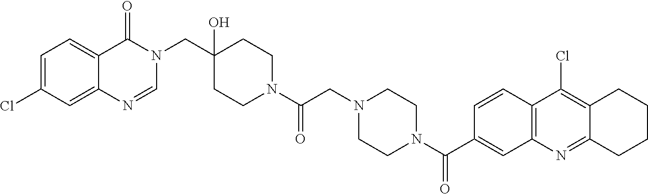 [SMILES: O=C(CN1CCN(C(=O)c2ccc3c(Cl)c4c(nc3c2)CCCC4)CC1)N1CCC(O)(Cn2cnc3cc(Cl)ccc3c2=O)CC1] |
| | 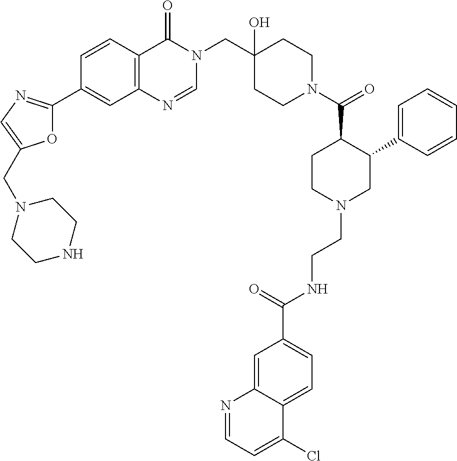 [SMILES: O=C(NCCN1CC[C@@H](C(=O)N2CCC(O)(Cn3cnc4cc(-c5ncc(CN6CCNCC6)o5)ccc4c3=O)CC2)[C@H](c2ccccc2)C1)c1ccc2c(Cl)ccnc2c1] |
| | 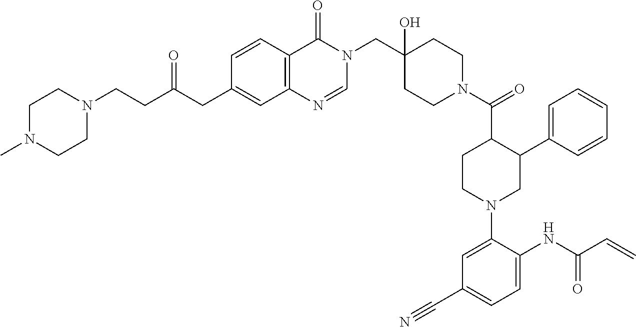 [SMILES: C=CC(=O)Nc1ccc(C#N)cc1N1CCC(C(=O)N2CCC(O)(Cn3cnc4cc(CC(=O)CCN5CCN(C)CC5)ccc4c3=O)CC2)C(c2ccccc2)C1] |
| | 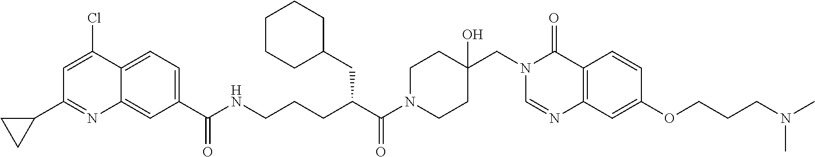 [SMILES: CN(C)CCCOc1ccc2c(=O)n(CC3(O)CCN(C(=O)[C@H](CCCNC(=O)c4ccc5c(Cl)cc(C6CC6)nc5c4)CC4CCCCC4)CC3)cnc2c1] |
| | 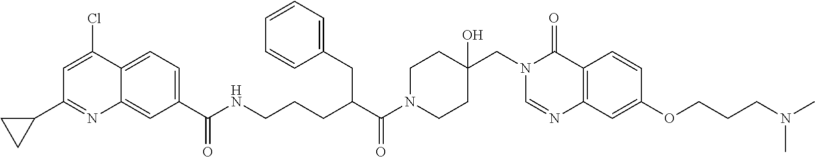 [SMILES: CN(C)CCCOc1ccc2c(=O)n(CC3(O)CCN(C(=O)C(CCCNC(=O)c4ccc5c(Cl)cc(C6CC6)nc5c4)Cc4ccccc4)CC3)cnc2c1] |
| | 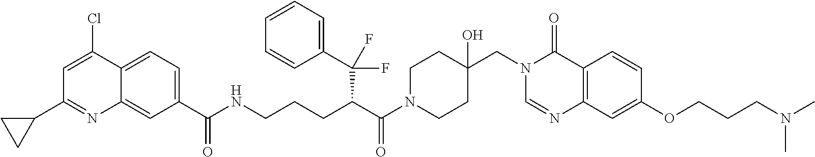 [SMILES: CN(C)CCCOc1ccc2c(=O)n(CC3(O)CCN(C(=O)[C@H](CCCNC(=O)c4ccc5c(Cl)cc(C6CC6)nc5c4)C(F)(F)c4ccccc4)CC3)cnc2c1] |
| | 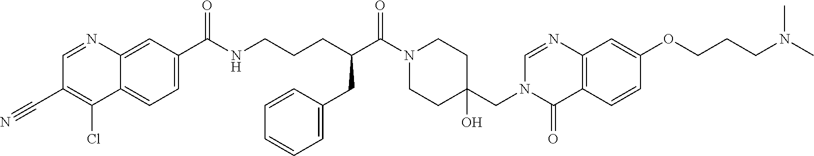 [SMILES: CN(C)CCCOc1ccc2c(=O)n(CC3(O)CCN(C(=O)[C@H](CCCNC(=O)c4ccc5c(Cl)c(C#N)cnc5c4)Cc4ccccc4)CC3)cnc2c1] |
| | 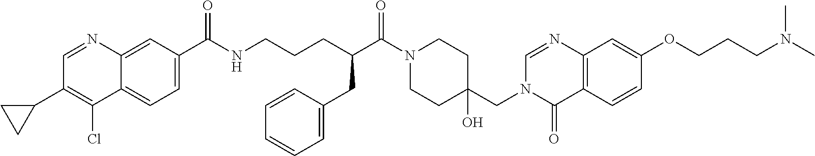 [SMILES: CN(C)CCCOc1ccc2c(=O)n(CC3(O)CCN(C(=O)[C@H](CCCNC(=O)c4ccc5c(Cl)c(C6CC6)cnc5c4)Cc4ccccc4)CC3)cnc2c1] |
| | 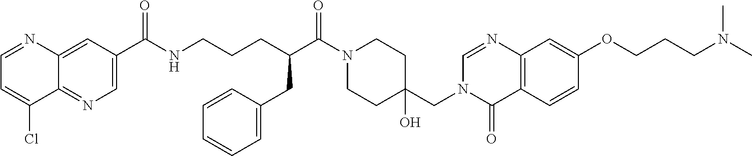 [SMILES: CN(C)CCCOc1ccc2c(=O)n(CC3(O)CCN(C(=O)[C@H](CCCNC(=O)c4cnc5c(Cl)ccnc5c4)Cc4ccccc4)CC3)cnc2c1] |
| | 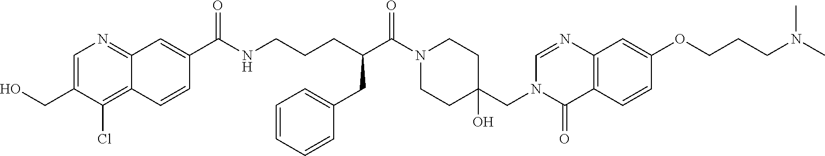 [SMILES: CN(C)CCCOc1ccc2c(=O)n(CC3(O)CCN(C(=O)[C@H](CCCNC(=O)c4ccc5c(Cl)c(CO)cnc5c4)Cc4ccccc4)CC3)cnc2c1] |
| | 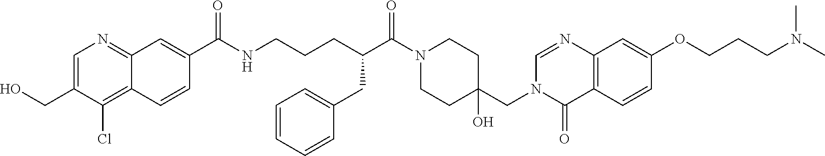 [SMILES: CN(C)CCCOc1ccc2c(=O)n(CC3(O)CCN(C(=O)[C@@H](CCCNC(=O)c4ccc5c(Cl)c(CO)cnc5c4)Cc4ccccc4)CC3)cnc2c1] |
| | 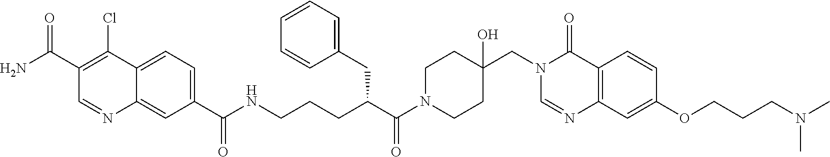 [SMILES: CN(C)CCCOc1ccc2c(=O)n(CC3(O)CCN(C(=O)[C@H](CCCNC(=O)c4ccc5c(Cl)c(C(N)=O)cnc5c4)Cc4ccccc4)CC3)cnc2c1] |
| | (stereochemistry not verified; opposite stereochemistry of next compound) |
| | 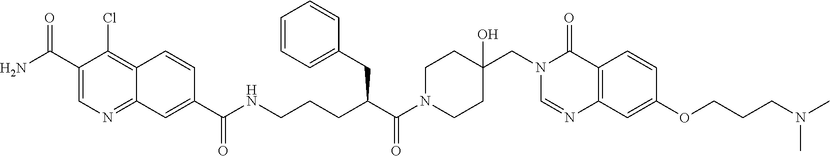 [SMILES: CN(C)CCCOc1ccc2c(=O)n(CC3(O)CCN(C(=O)[C@@H](CCCNC(=O)c4ccc5c(Cl)c(C(N)=O)cnc5c4)Cc4ccccc4)CC3)cnc2c1] |
| | (stereochemistry not verified; opposite stereochmistry of previous compound) |
| | 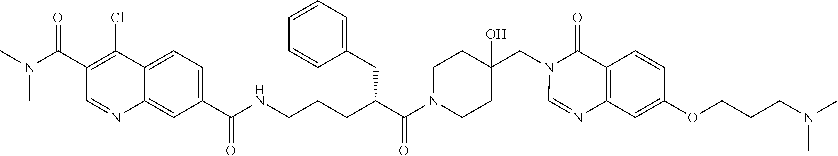 |
| | 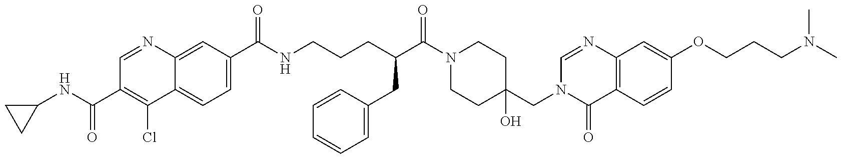 [SMILES: CN(C)CCCOc1ccc2c(=O)n(CC3(O)CCN(C(=O)[C@H](CCCNC(=O)c4ccc5c(Cl)c(C(=O)NC6CC6)cnc5c4)Cc4ccccc4)CC3)cnc2c1] |
| | (stereochemistry not verified; opposite stereochemistry of next compound) |
| | 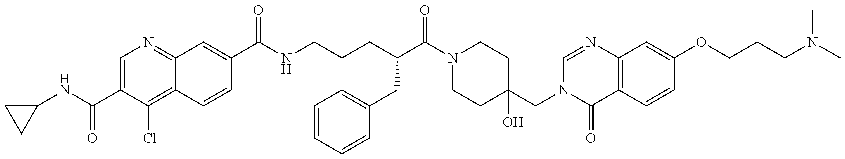 [SMILES: CN(C)CCCOc1ccc2c(=O)n(CC3(O)CCN(C(=O)[C@@H](CCCNC(=O)c4ccc5c(Cl)c(C(=O)NC6CC6)cnc5c4)Cc4ccccc4)CC3)cnc2c1] |
| | (stereochemistry not verified; opposite stereochemistry of previous compound) |
| | 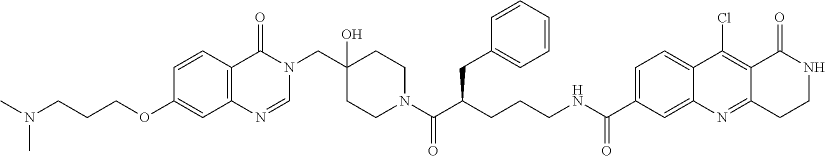 [SMILES: CN(C)CCCOc1ccc2c(=O)n(CC3(O)CCN(C(=O)[C@H](CCCNC(=O)c4ccc5c(Cl)c6c(nc5c4)CCNC6=O)Cc4ccccc4)CC3)cnc2c1] |
| | 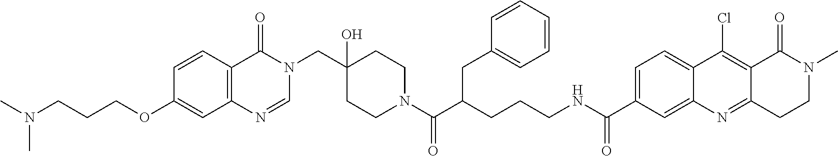 |
| | 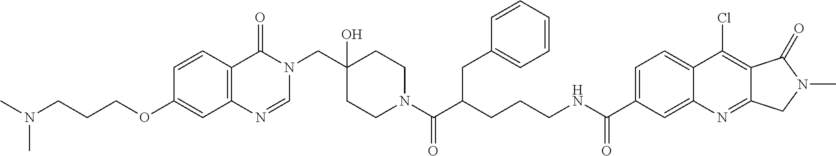 |
| | 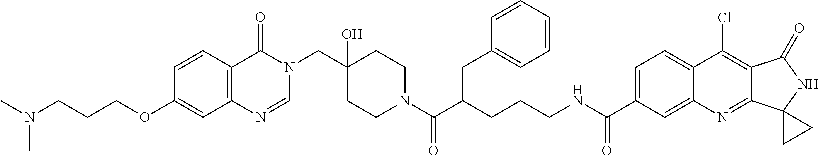 [SMILES: CN(C)CCCOc1ccc2c(=O)n(CC3(O)CCN(C(=O)C(CCCNC(=O)c4ccc5c(Cl)c6c(nc5c4)C4(CC4)NC6=O)Cc4ccccc4)CC3)cnc2c1] |
| | 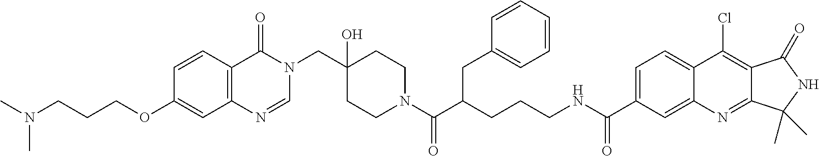 |
| | 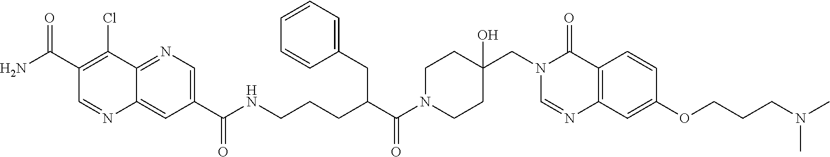 [SMILES: CN(C)CCCOc1ccc2c(=O)n(CC3(O)CCN(C(=O)C(CCCNC(=O)c4cnc5c(Cl)c(C(N)=O)cnc5c4)Cc4ccccc4)CC3)cnc2c1] |
| | 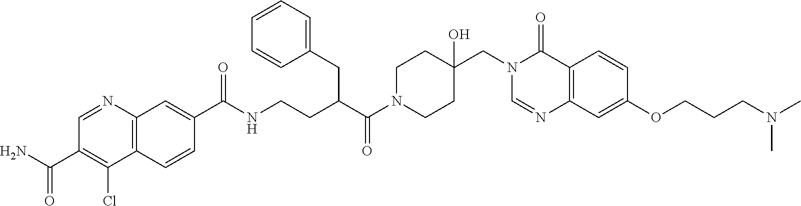 [SMILES: CN(C)CCCOc1ccc2c(=O)n(CC3(O)CCN(C(=O)C(CCNC(=O)c4ccc5c(Cl)c(C(N)=O)cnc5c4)Cc4ccccc4)CC3)cnc2c1] |
| | 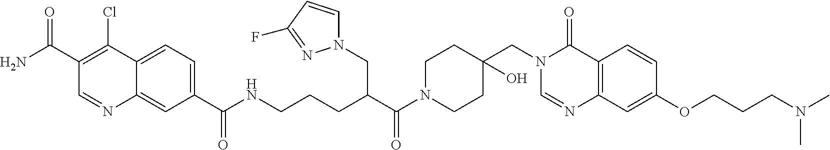 [SMILES: CN(C)CCCOc1ccc2c(=O)n(CC3(O)CCN(C(=O)C(CCCNC(=O)c4ccc5c(Cl)c(C(N)=O)cnc5c4)Cn4ccc(F)n4)CC3)cnc2c1] |
| | 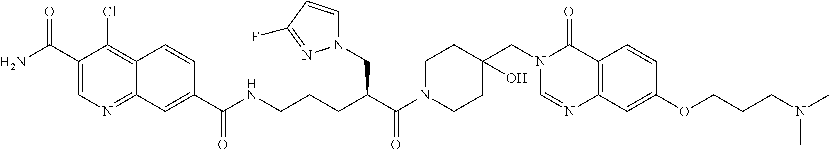 [SMILES: CN(C)CCCOc1ccc2c(=O)n(CC3(O)CCN(C(=O)[C@@H](CCCNC(=O)c4ccc5c(Cl)c(C(N)=O)cnc5c4)Cn4ccc(F)n4)CC3)cnc2c1] |
| | 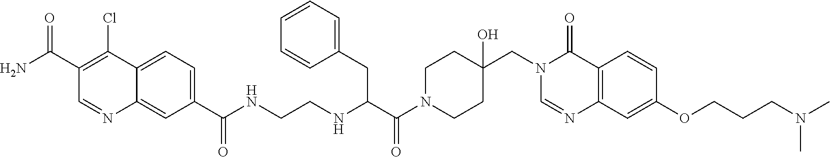 [SMILES: CN(C)CCCOc1ccc2c(=O)n(CC3(O)CCN(C(=O)C(Cc4ccccc4)NCCNC(=O)c4ccc5c(Cl)c(C(N)=O)cnc5c4)CC3)cnc2c1] |
| | 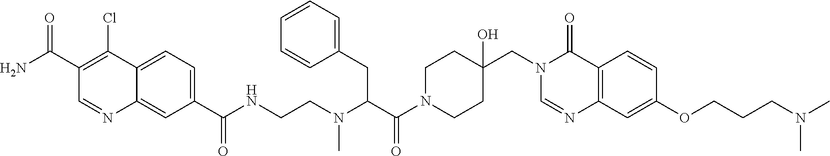 [SMILES: CN(C)CCCOc1ccc2c(=O)n(CC3(O)CCN(C(=O)C(Cc4ccccc4)N(C)CCNC(=O)c4ccc5c(Cl)c(C(N)=O)cnc5c4)CC3)cnc2c1] |
| | 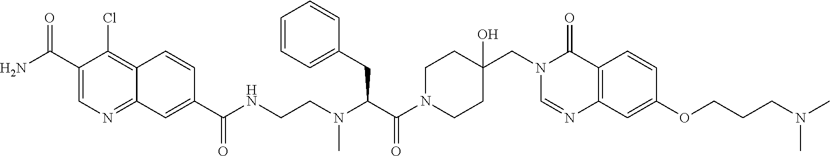 [SMILES: CN(C)CCCOc1ccc2c(=O)n(CC3(O)CCN(C(=O)[C@H](Cc4ccccc4)N(C)CCNC(=O)c4ccc5c(Cl)c(C(N)=O)cnc5c4)CC3)cnc2c1] |
| | 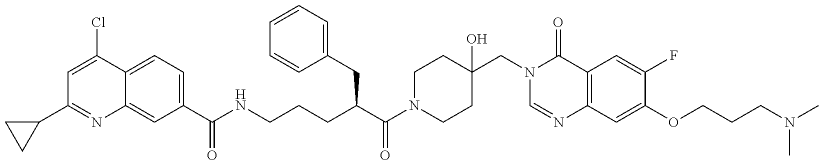 [SMILES: CN(C)CCCOc1cc2ncn(CC3(O)CCN(C(=O)[C@@H](CCCNC(=O)c4ccc5c(Cl)cc(C6CC6)nc5c4)Cc4ccccc4)CC3)c(=O)c2cc1F] |
| | 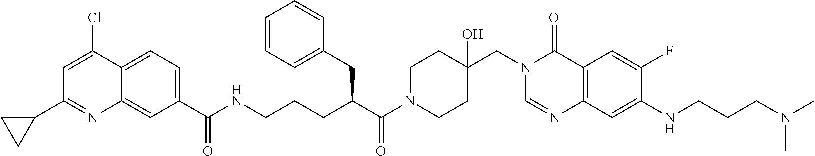 [SMILES: CN(C)CCCNc1cc2ncn(CC3(O)CCN(C(=O)[C@@H](CCCNC(=O)c4ccc5c(Cl)cc(C6CC6)nc5c4)Cc4ccccc4)CC3)c(=O)c2cc1F] |
| | 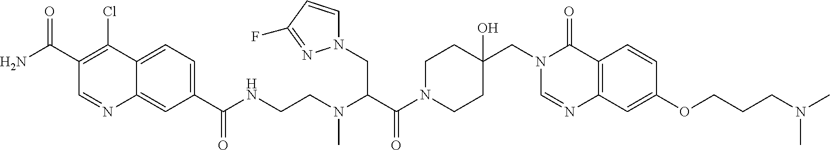 [SMILES: CN(C)CCCOc1ccc2c(=O)n(CC3(O)CCN(C(=O)C(Cn4ccc(F)n4)N(C)CCNC(=O)c4ccc5c(Cl)c(C(N)=O)cnc5c4)CC3)cnc2c1] |
| | 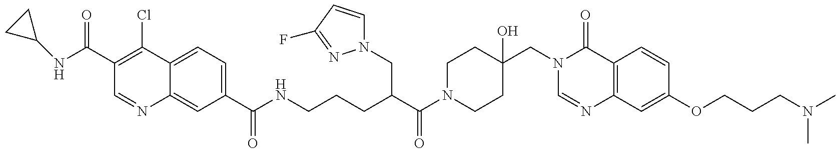 [SMILES: CN(C)CCCOc1ccc2c(=O)n(CC3(O)CCN(C(=O)C(CCCNC(=O)c4ccc5c(Cl)c(C(=O)NC6CC6)cnc5c4)Cn4ccc(F)n4)CC3)cnc2c1] |
| | 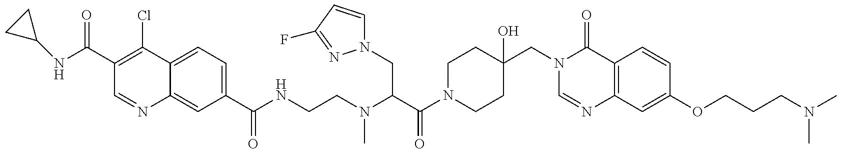 [SMILES: CN(C)CCCOc1ccc2c(=O)n(CC3(O)CCN(C(=O)C(Cn4ccc(F)n4)N(C)CCNC(=O)c4ccc5c(Cl)c(C(=O)NC6CC6)cnc5c4)CC3)cnc2c1] |
| | 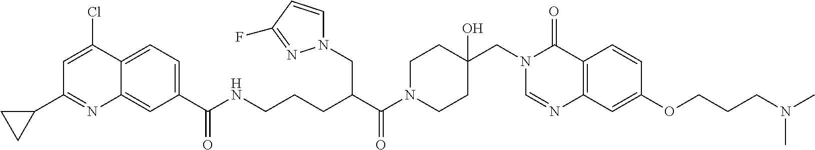 [SMILES: CN(C)CCCOc1ccc2c(=O)n(CC3(O)CCN(C(=O)C(CCCNC(=O)c4ccc5c(Cl)cc(C6CC6)nc5c4)Cn4ccc(F)n4)CC3)cnc2c1] |
| | 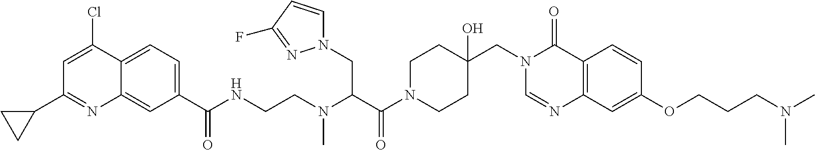 [SMILES: CN(C)CCCOc1ccc2c(=O)n(CC3(O)CCN(C(=O)C(Cn4ccc(F)n4)N(C)CCNC(=O)c4ccc5c(Cl)cc(C6CC6)nc5c4)CC3)cnc2c1] |
| | 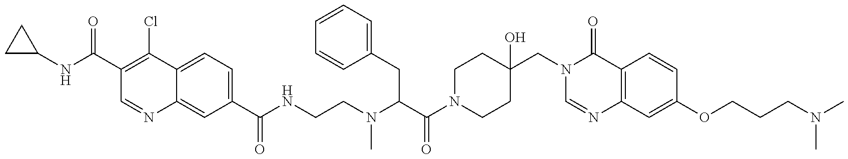 [SMILES: CN(C)CCCOc1ccc2c(=O)n(CC3(O)CCN(C(=O)C(Cc4ccccc4)N(C)CCNC(=O)c4ccc5c(Cl)c(C(=O)NC6CC6)cnc5c4)CC3)cnc2c1] |
| | 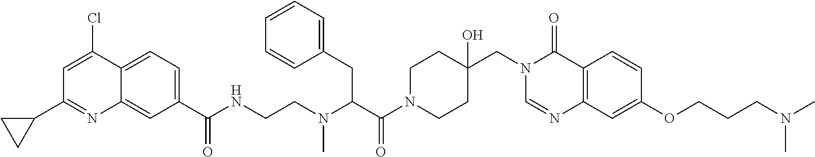 [SMILES: CN(C)CCCOc1ccc2c(=O)n(CC3(O)CCN(C(=O)C(Cc4ccccc4)N(C)CCNC(=O)c4ccc5c(Cl)cc(C6CC6)nc5c4)CC3)cnc2c1] |
| | 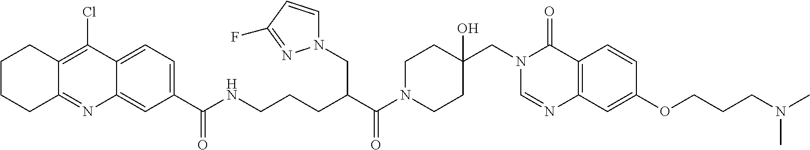 [SMILES: CN(C)CCCOc1ccc2c(=O)n(CC3(O)CCN(C(=O)C(CCCNC(=O)c4ccc5c(Cl)c6c(nc5c4)CCCC6)Cn4ccc(F)n4)CC3)cnc2c1] |
| | 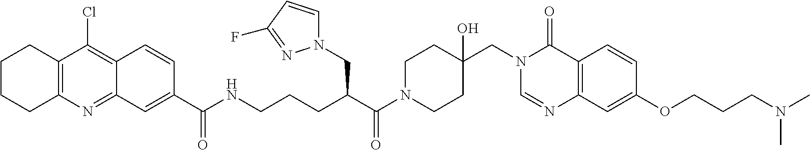 [SMILES: CN(C)CCCOc1ccc2c(=O)n(CC3(O)CCN(C(=O)[C@@H](CCCNC(=O)c4ccc5c(Cl)c6c(nc5c4)CCCC6)Cn4ccc(F)n4)CC3)cnc2c1] |
| | 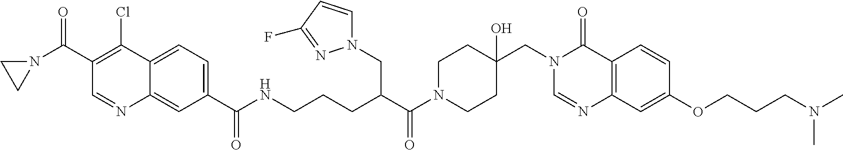 [SMILES: CN(C)CCCOc1ccc2c(=O)n(CC3(O)CCN(C(=O)C(CCCNC(=O)c4ccc5c(Cl)c(C(=O)N6CC6)cnc5c4)Cn4ccc(F)n4)CC3)cnc2c1] |
| | 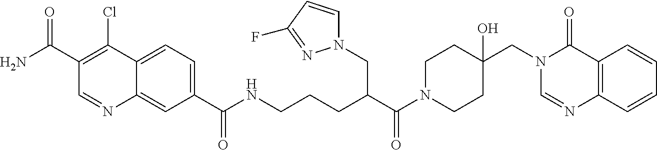 [SMILES: NC(=O)c1cnc2cc(C(=O)NCCCC(Cn3ccc(F)n3)C(=O)N3CCC(O)(Cn4cnc5ccccc5c4=O)CC3)ccc2c1Cl] |
| | 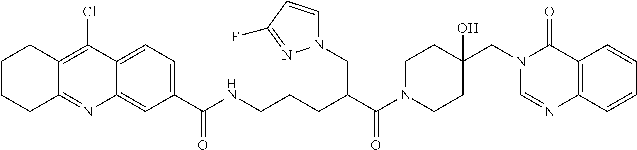 [SMILES: O=C(NCCCC(Cn1ccc(F)n1)C(=O)N1CCC(O)(Cn2cnc3ccccc3c2=O)CC1)c1ccc2c(Cl)c3c(nc2c1)CCCC3] |
| | 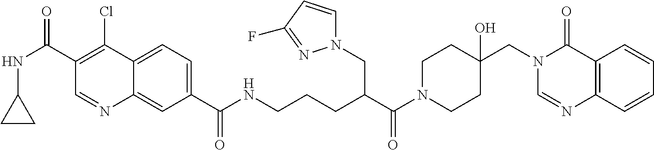 [SMILES: O=C(NCCCC(Cn1ccc(F)n1)C(=O)N1CCC(O)(Cn2cnc3ccccc3c2=O)CC1)c1ccc2c(Cl)c(C(=O)NC3CC3)cnc2c1] |
| | 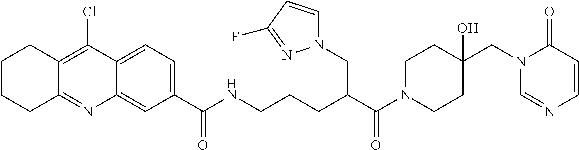 [SMILES: O=C(NCCCC(Cn1ccc(F)n1)C(=O)N1CCC(O)(Cn2cnccc2=O)CC1)c1ccc2c(Cl)c3c(nc2c1)CCCC3] |

In some embodiments, the compound of formula (I) is selected from:

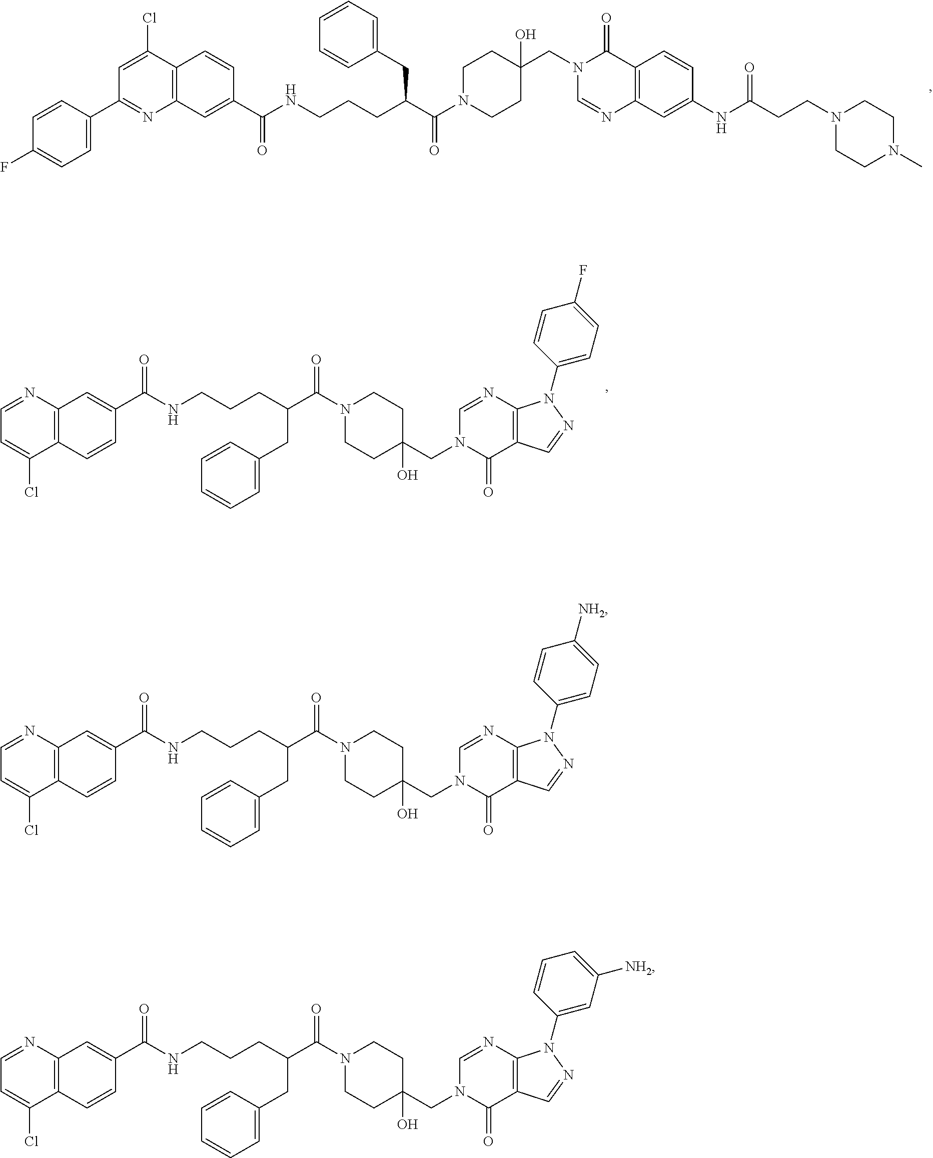 [SMILES: CN1CCN(CCC(=O)Nc2ccc3c(=O)n(CC4(O)CCN(C(=O)[C@@H](CCCNC(=O)c5ccc6c(Cl)cc(-c7ccc(F)cc7)nc6c5)Cc5ccccc5)CC4)cnc3c2)CC1 ; O=C(NCCCC(Cc1ccccc1)C(=O)N1CCC(O)(Cn2cnc3c(cnn3-c3ccc(F)cc3)c2=O)CC1)c1ccc2c(Cl)ccnc2c1 ; Nc1ccc(-n2ncc3c(=O)n(CC4(O)CCN(C(=O)C(CCCNC(=O)c5ccc6c(Cl)ccnc6c5)Cc5ccccc5)CC4)cnc32)cc1 ; Nc1cccc(-n2ncc3c(=O)n(CC4(O)CCN(C(=O)C(CCCNC(=O)c5ccc6c(Cl)ccnc6c5)Cc5ccccc5)CC4)cnc32)c1]

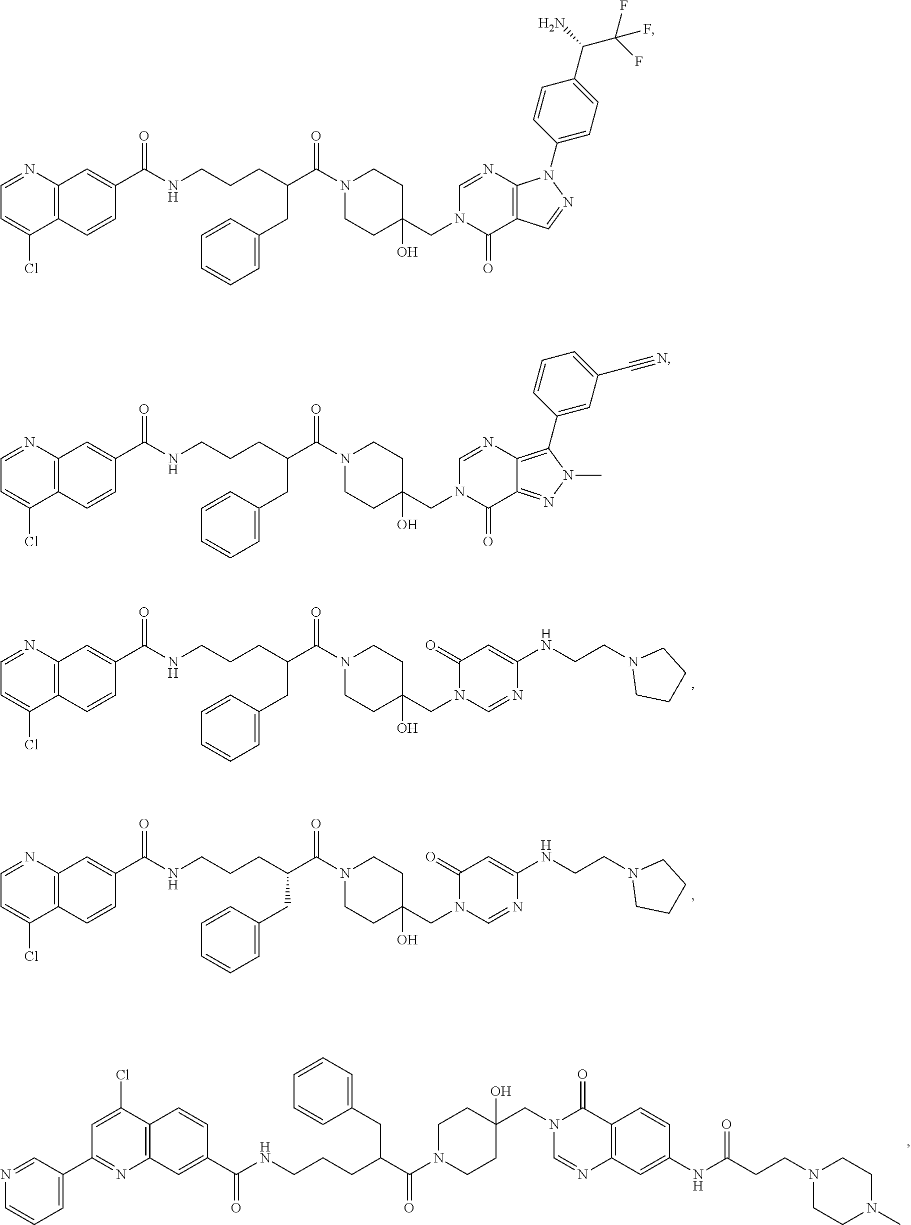 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F ; Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1 ; O=C(NCCCC(Cc1ccccc1)C(=O)N1CCC(O)(Cn2cnc(NCCN3CCCC3)cc2=O)CC1)c1ccc2c(Cl)ccnc2c1 ; O=C(NCCC[C@@H](Cc1ccccc1)C(=O)N1CCC(O)(Cn2cnc(NCCN3CCCC3)cc2=O)CC1)c1ccc2c(Cl)ccnc2c1 ; CN1CCN(CCC(=O)Nc2ccc3c(=O)n(CC4(O)CCN(C(=O)C(CCCNC(=O)c5ccc6c(Cl)cc(-c7cccnc7)nc6c5)Cc5ccccc5)CC4)cnc3c2)CC1]

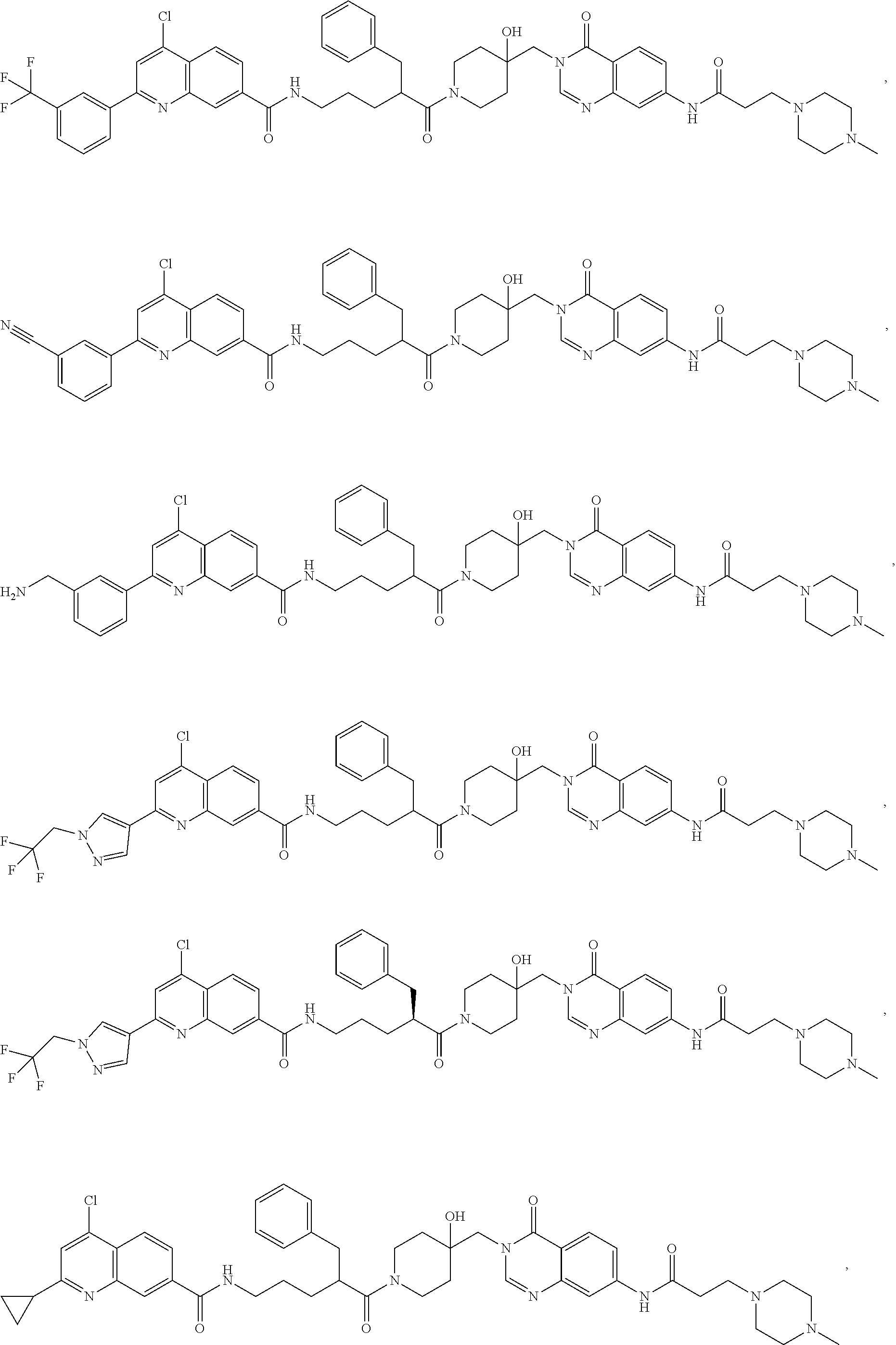

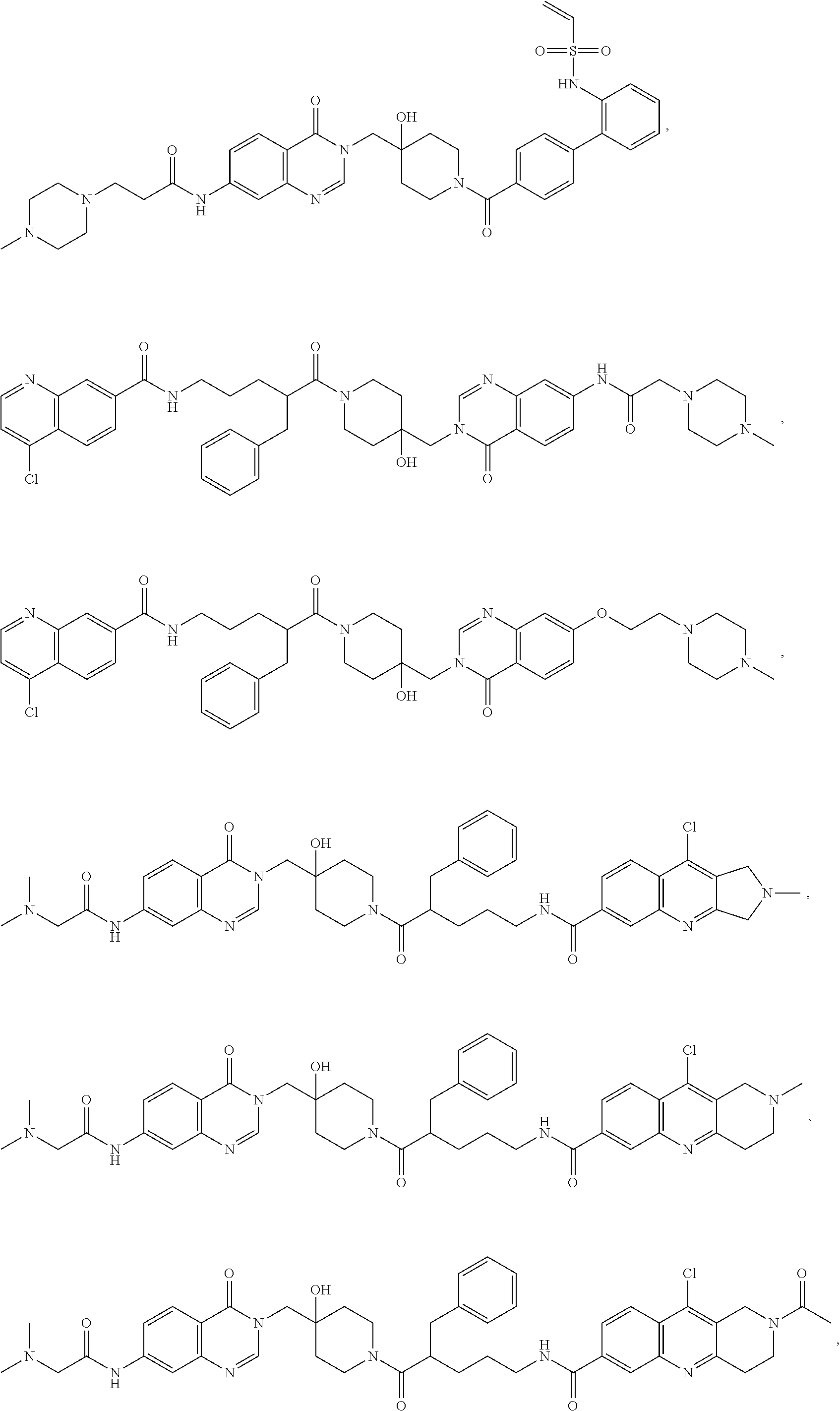

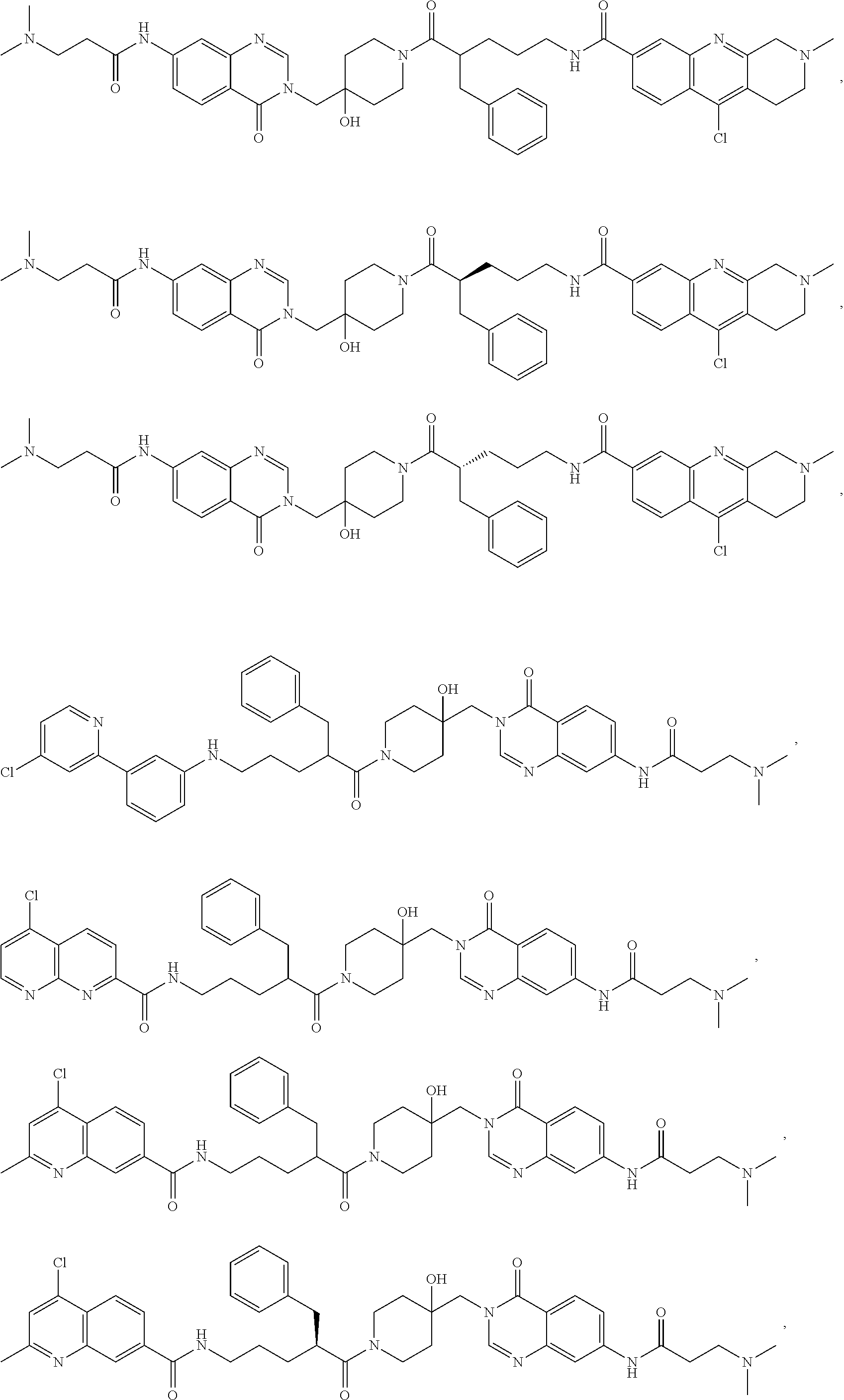

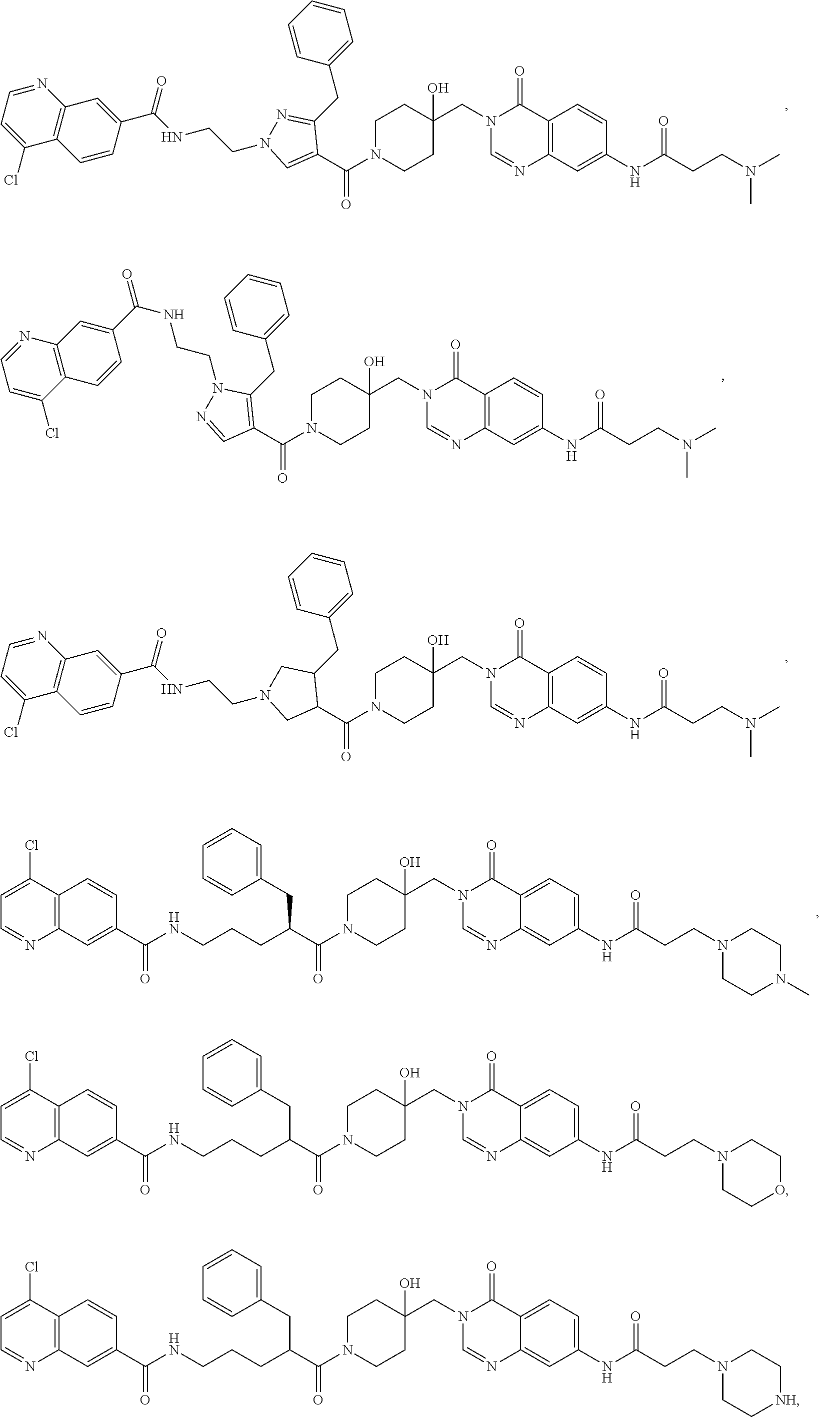

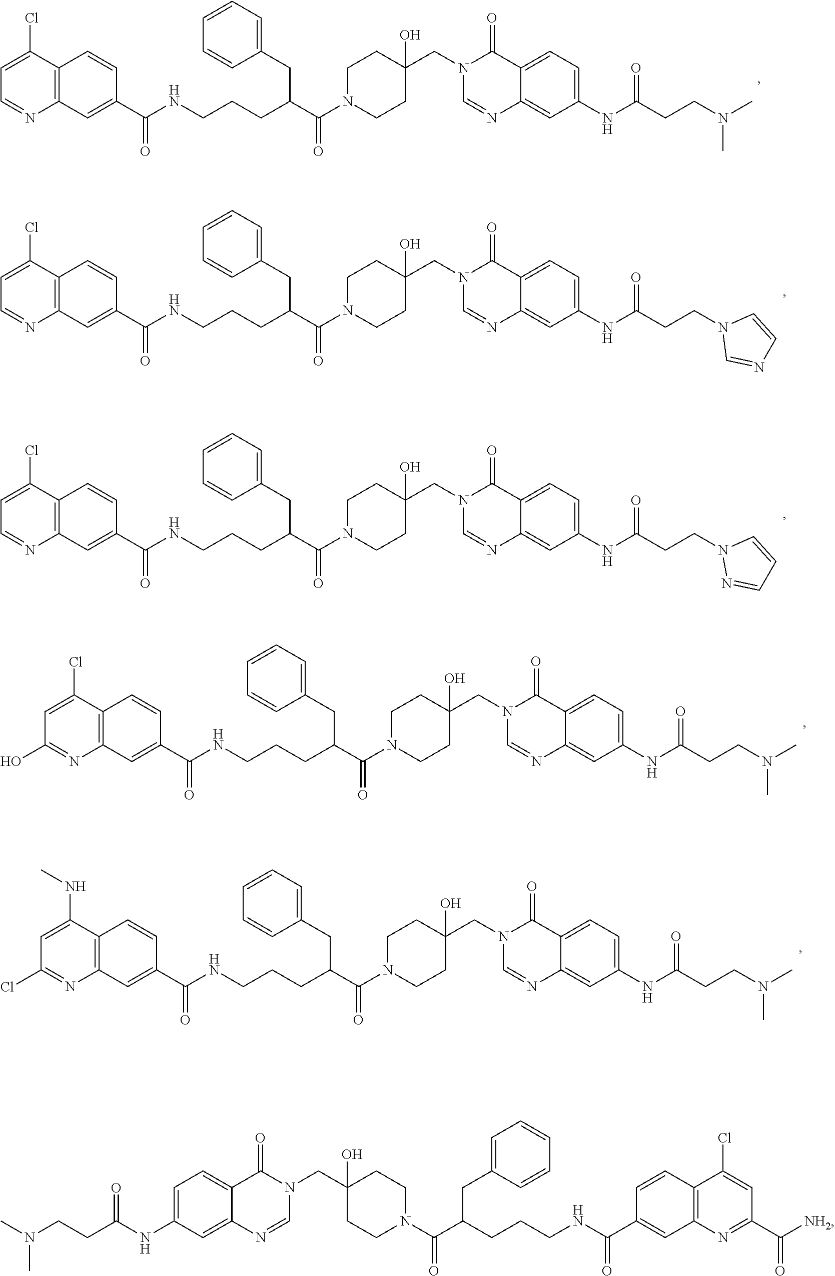

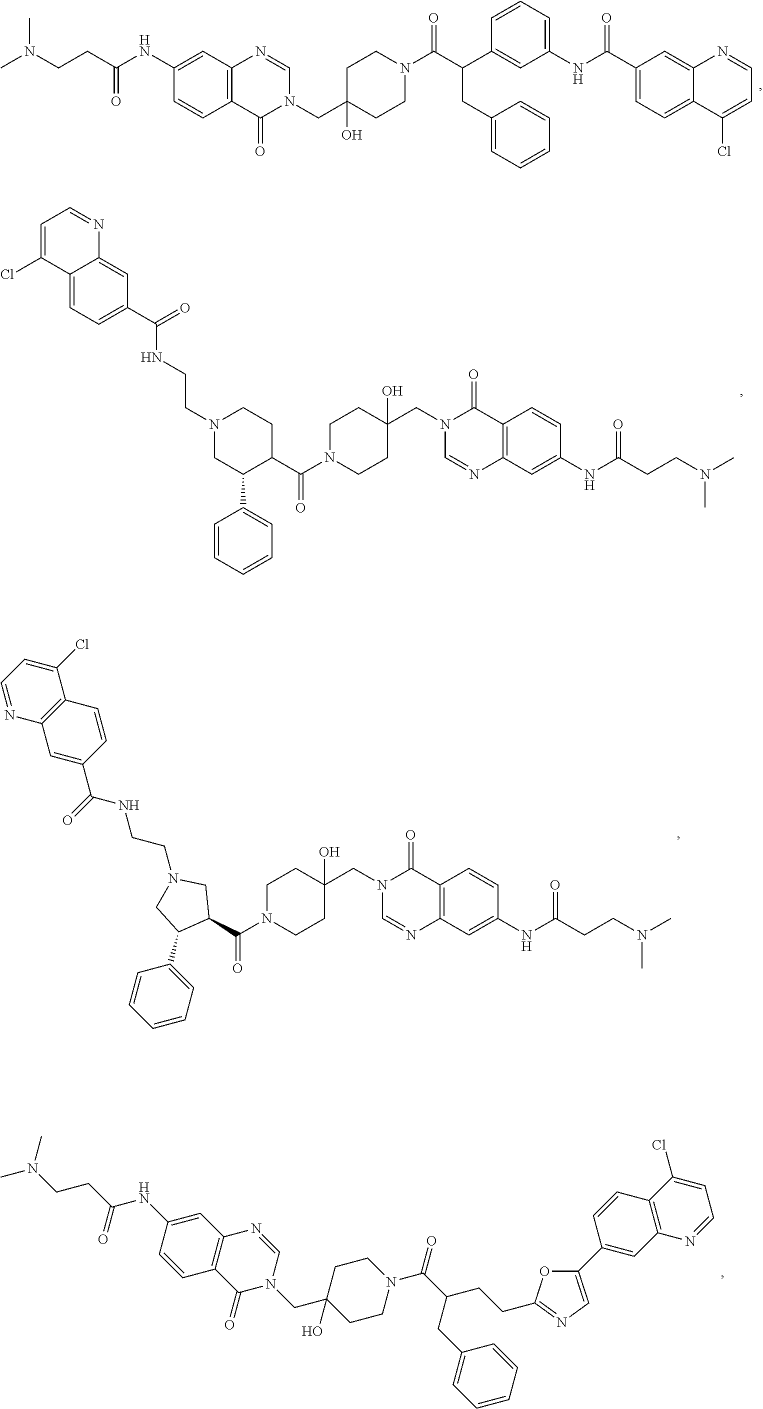

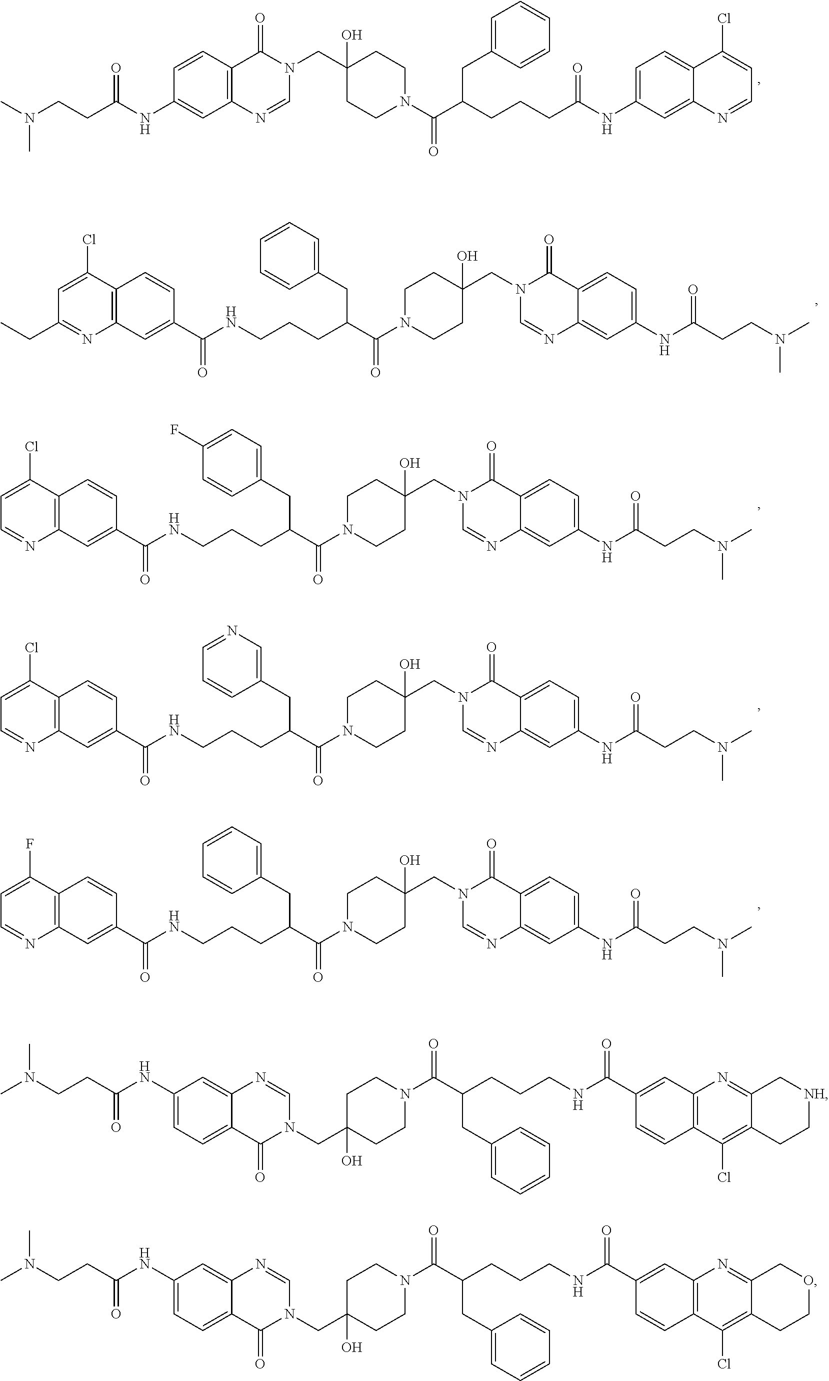

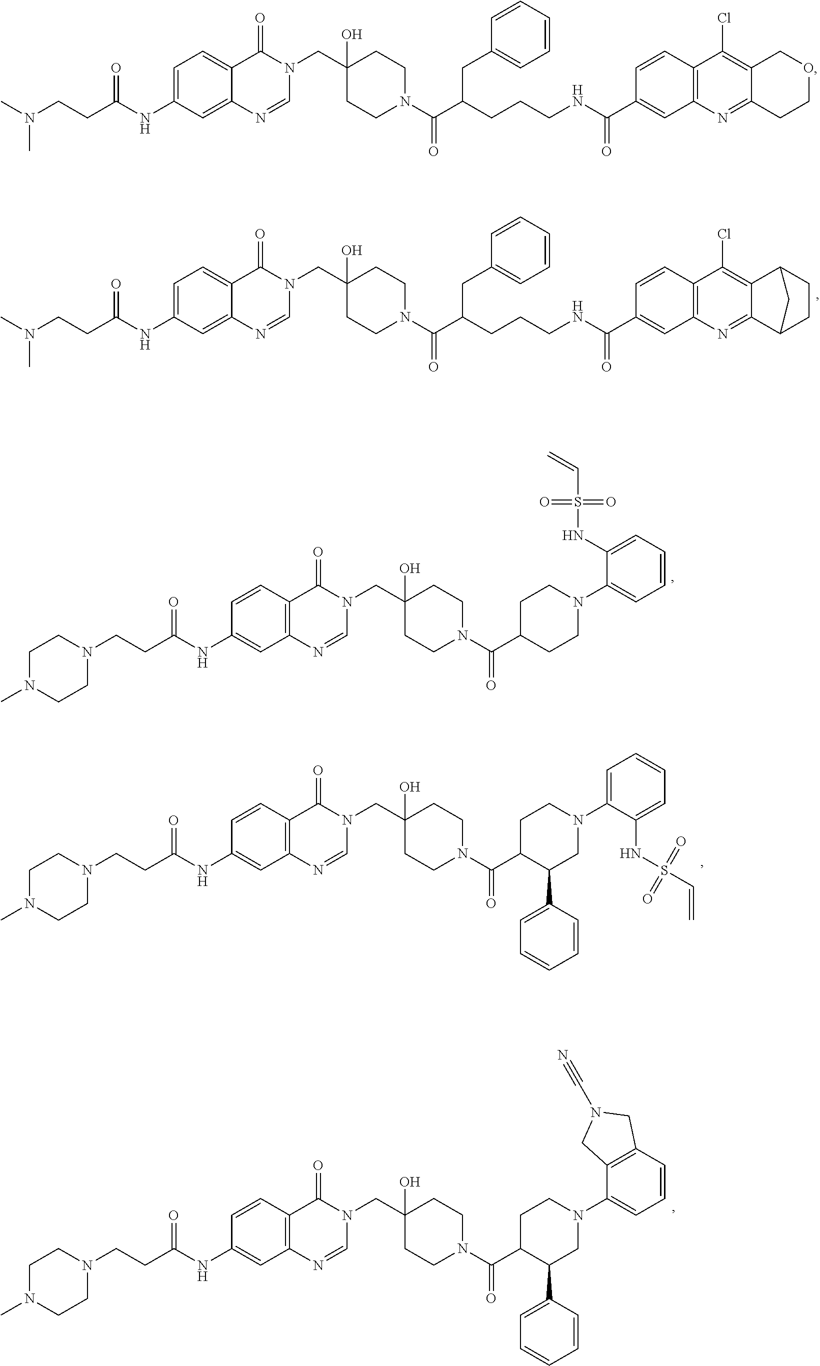

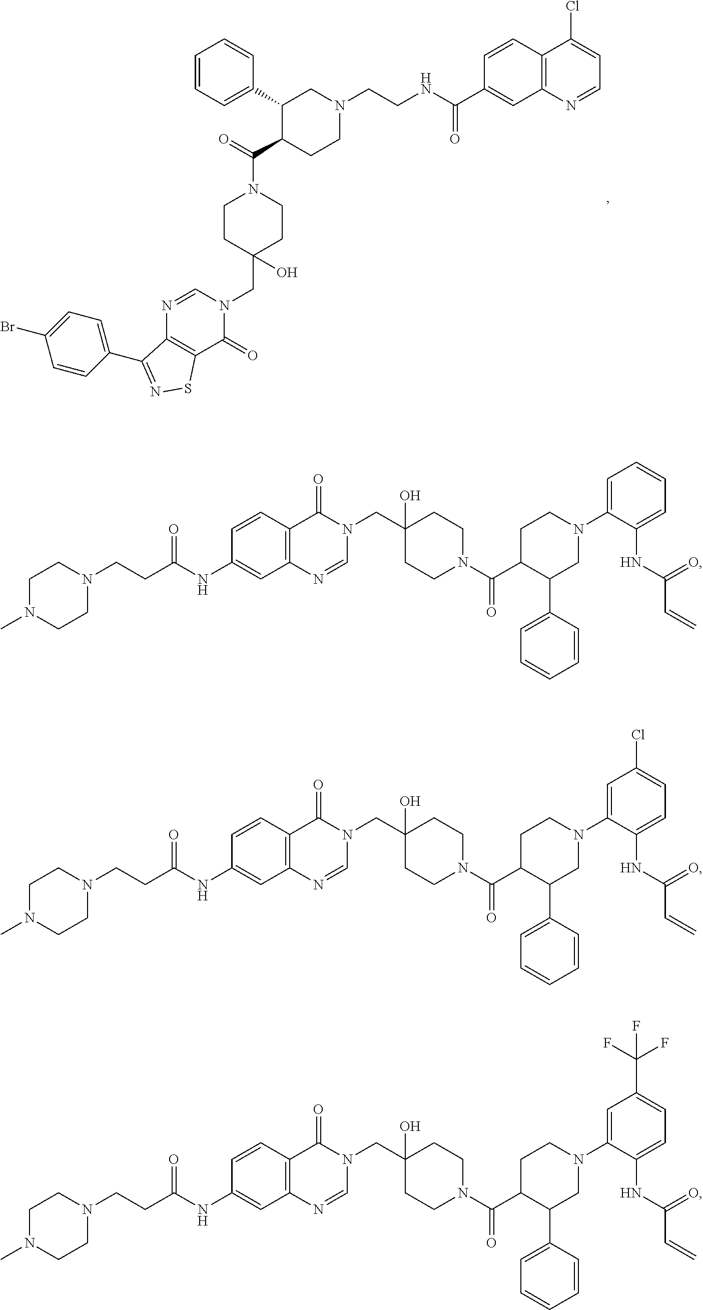

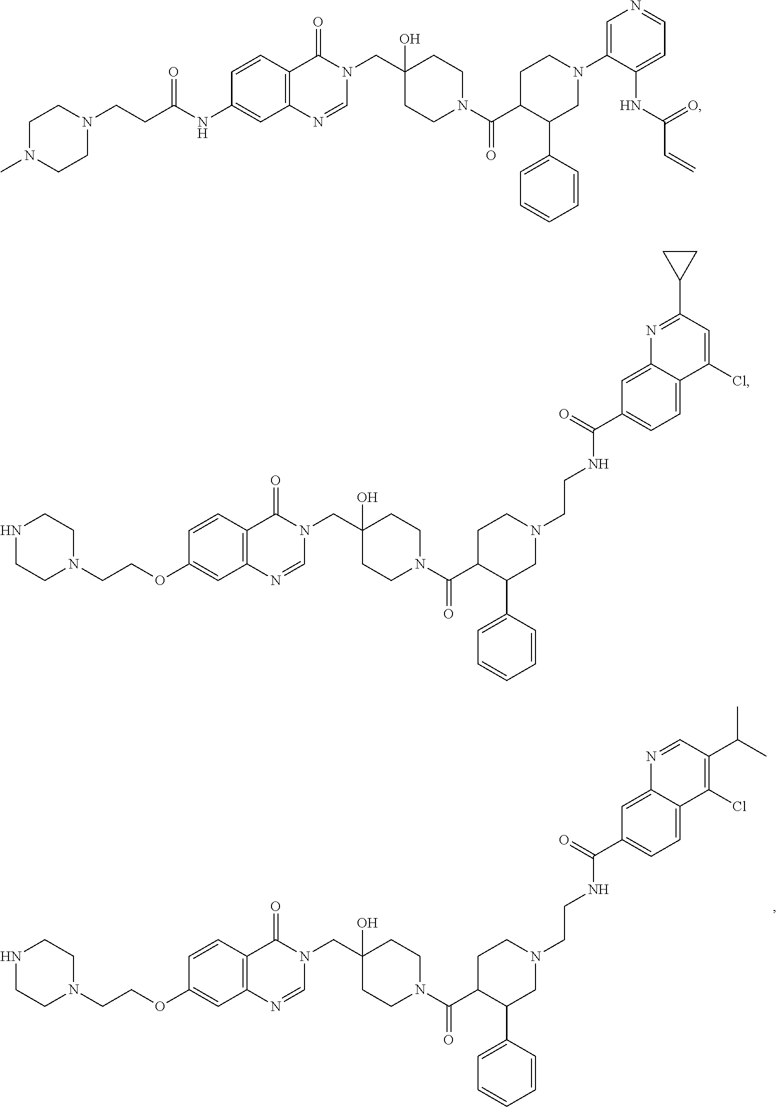 [SMILES: C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1 ; O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1 ; CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl]

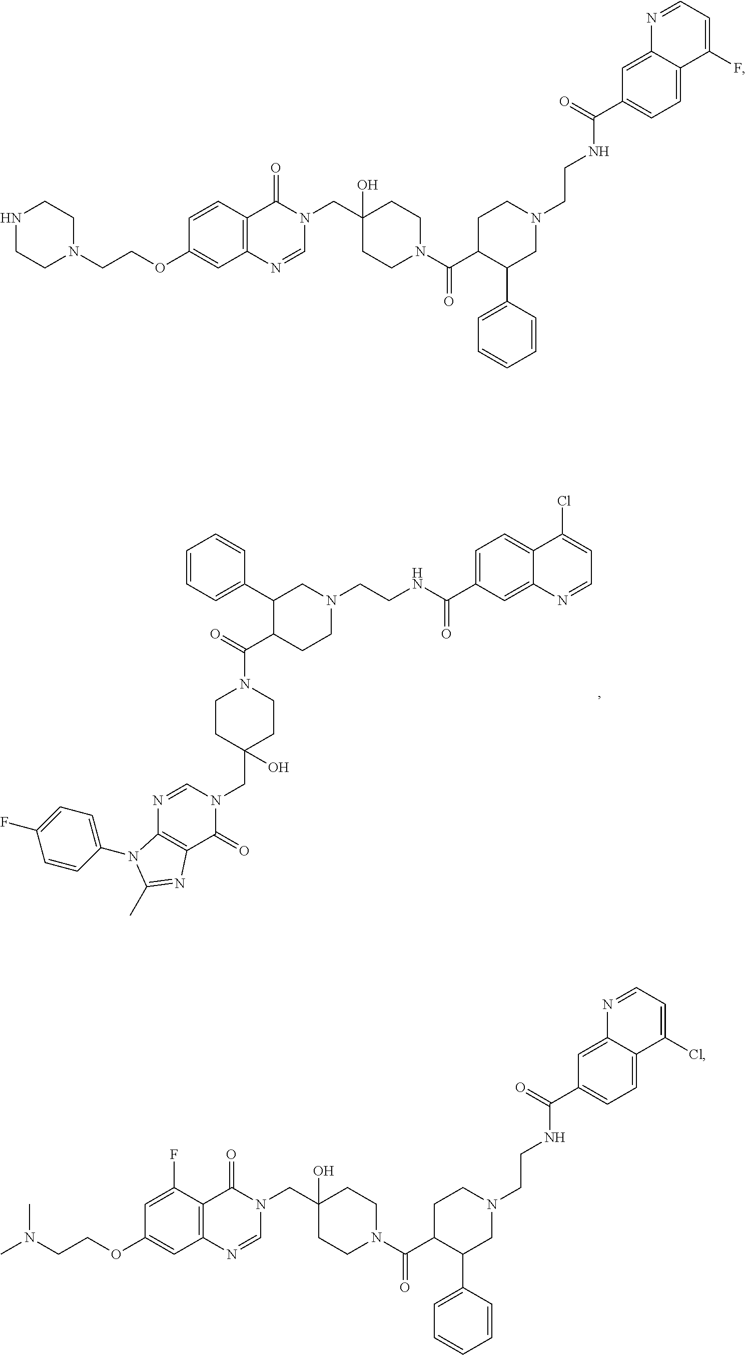 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1 ; Cc1nc2c(=O)n(CC3(O)CCN(C(=O)C4CCN(CCNC(=O)c5ccc6c(Cl)ccnc6c5)CC4c4ccccc4)CC3)cnc2n1-c1ccc(F)cc1 ; CN(C)CCOc1cc(F)c2c(=O)n(CC3(O)CCN(C(=O)C4CCN(CCNC(=O)c5ccc6c(Cl)ccnc6c5)CC4c4ccccc4)CC3)cnc2c1]

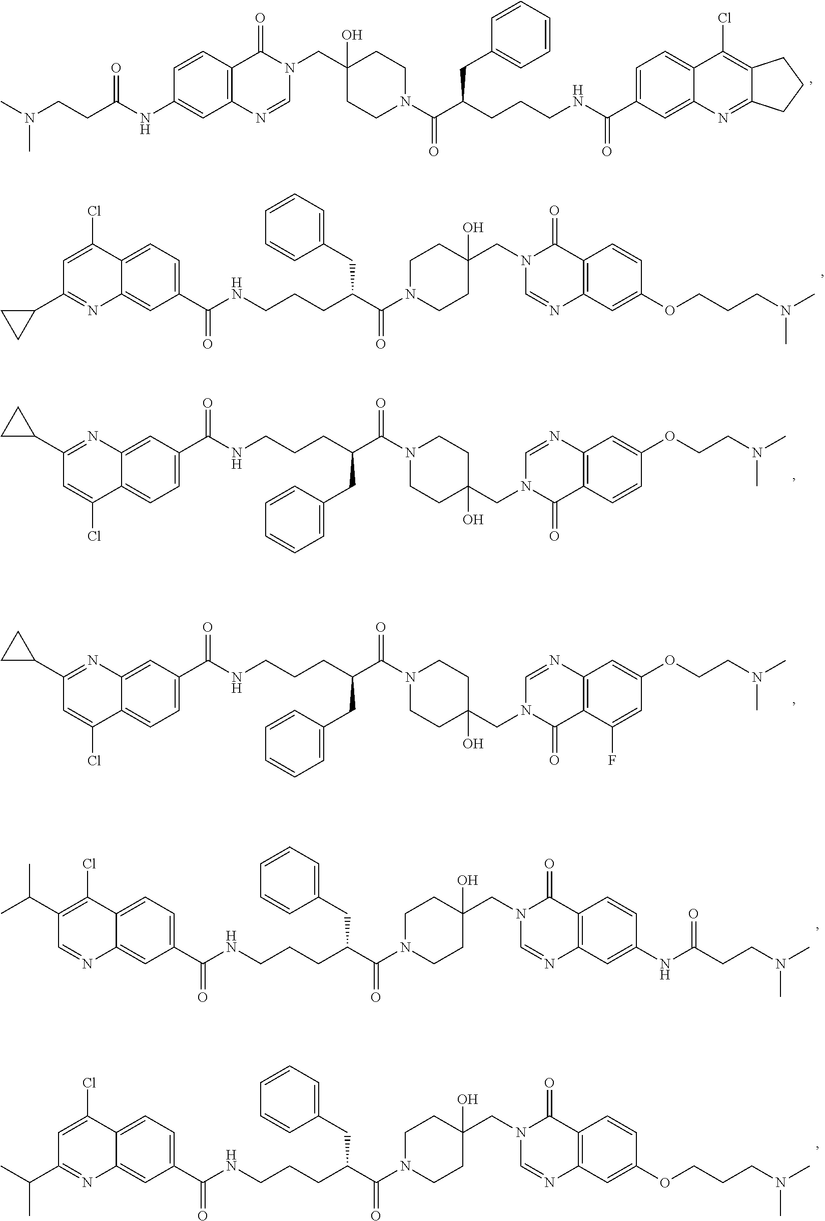

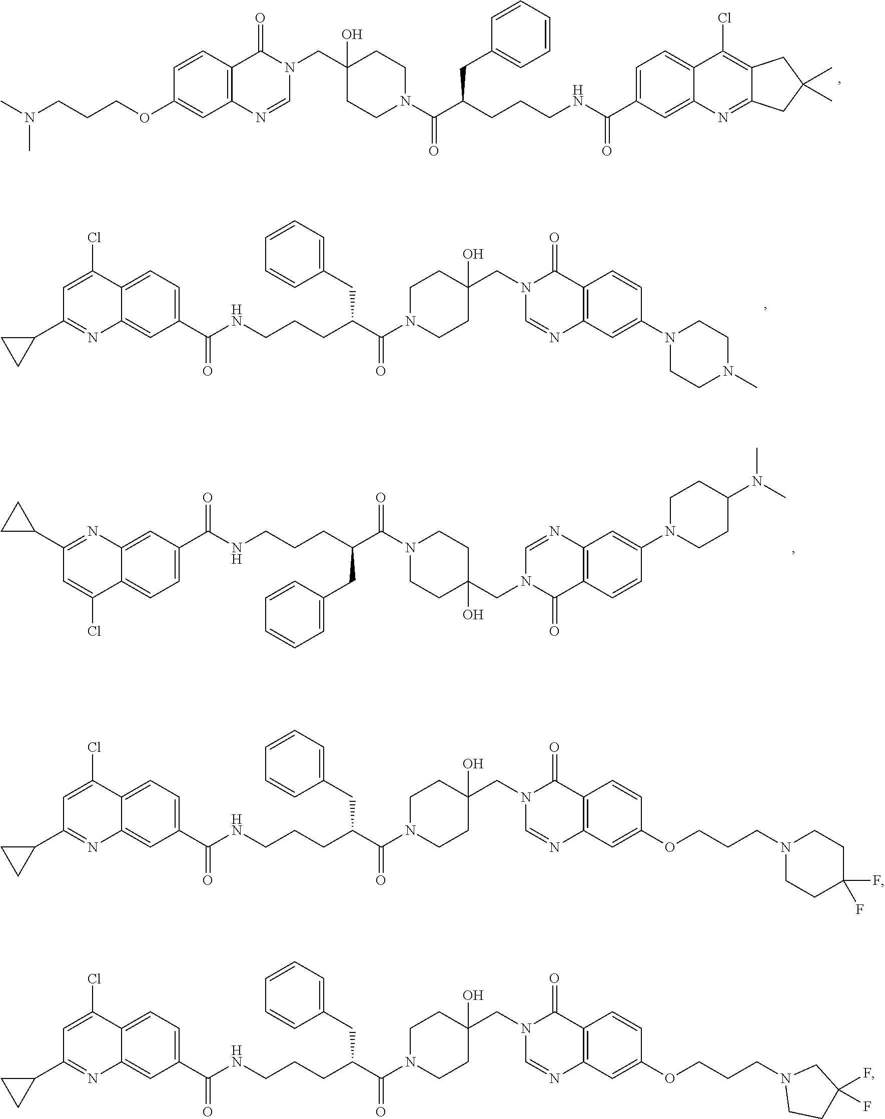

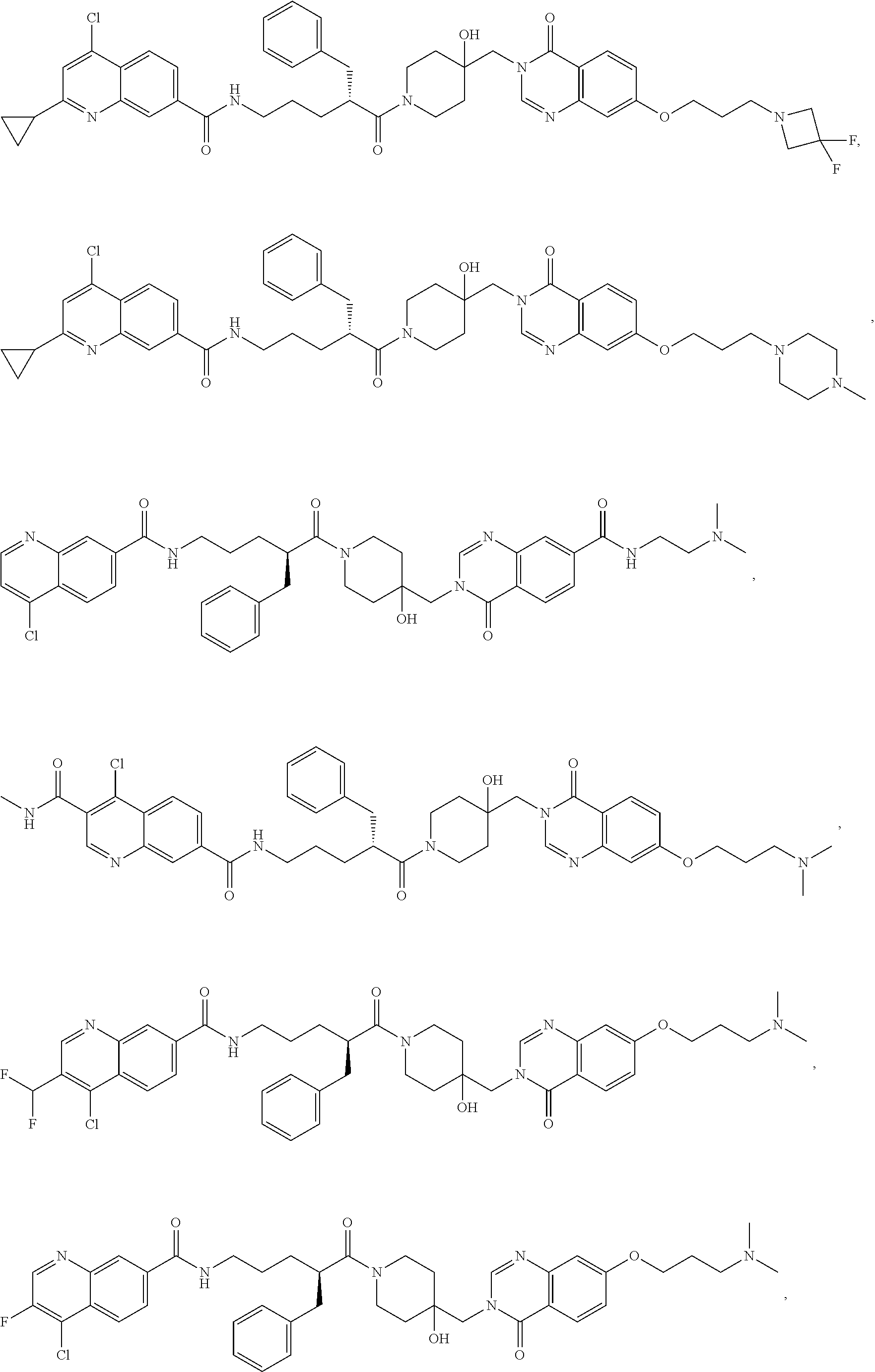

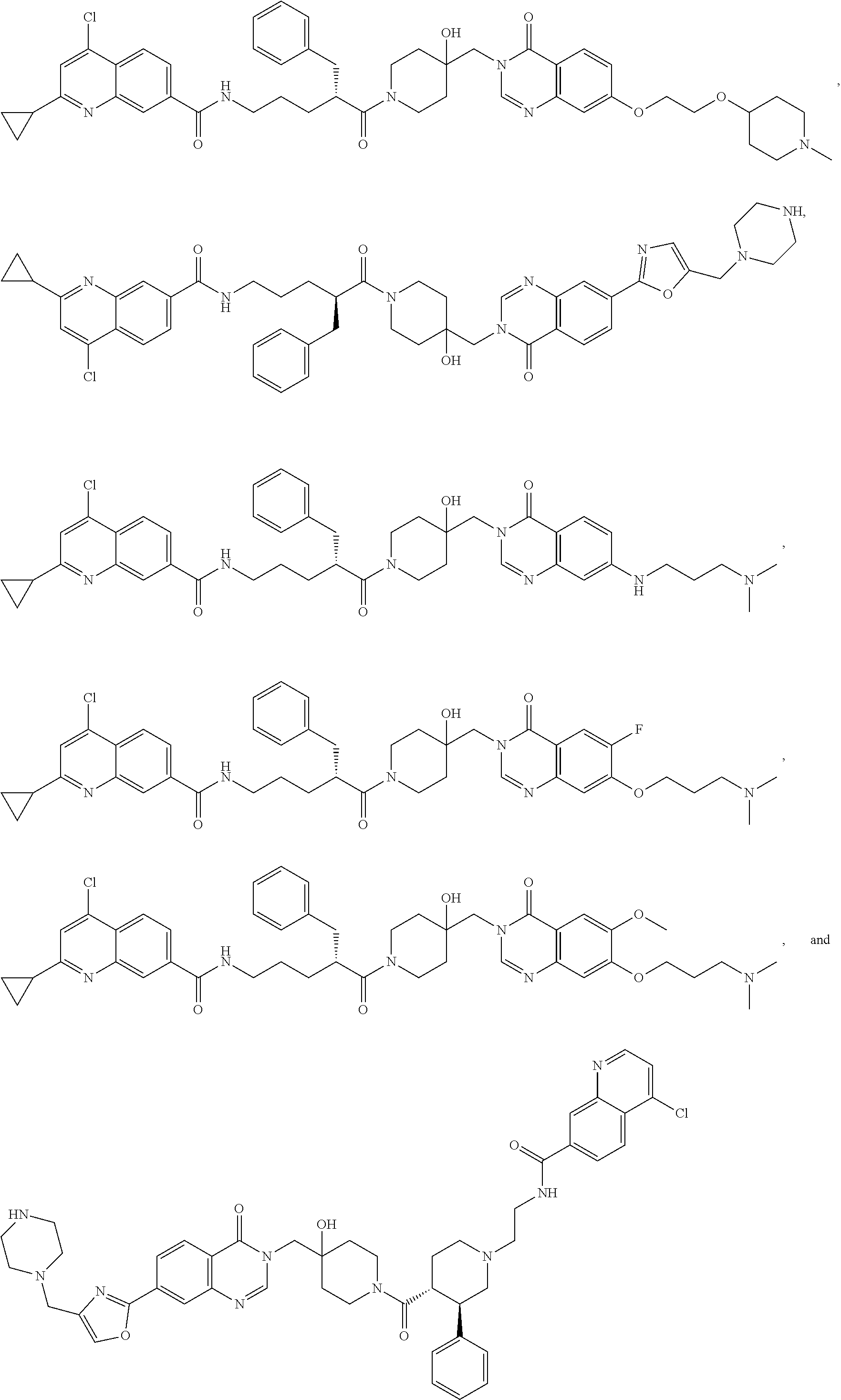

or a pharmaceutically acceptable salt thereof.

In some embodiments, the compound of formula (I) is selected from:

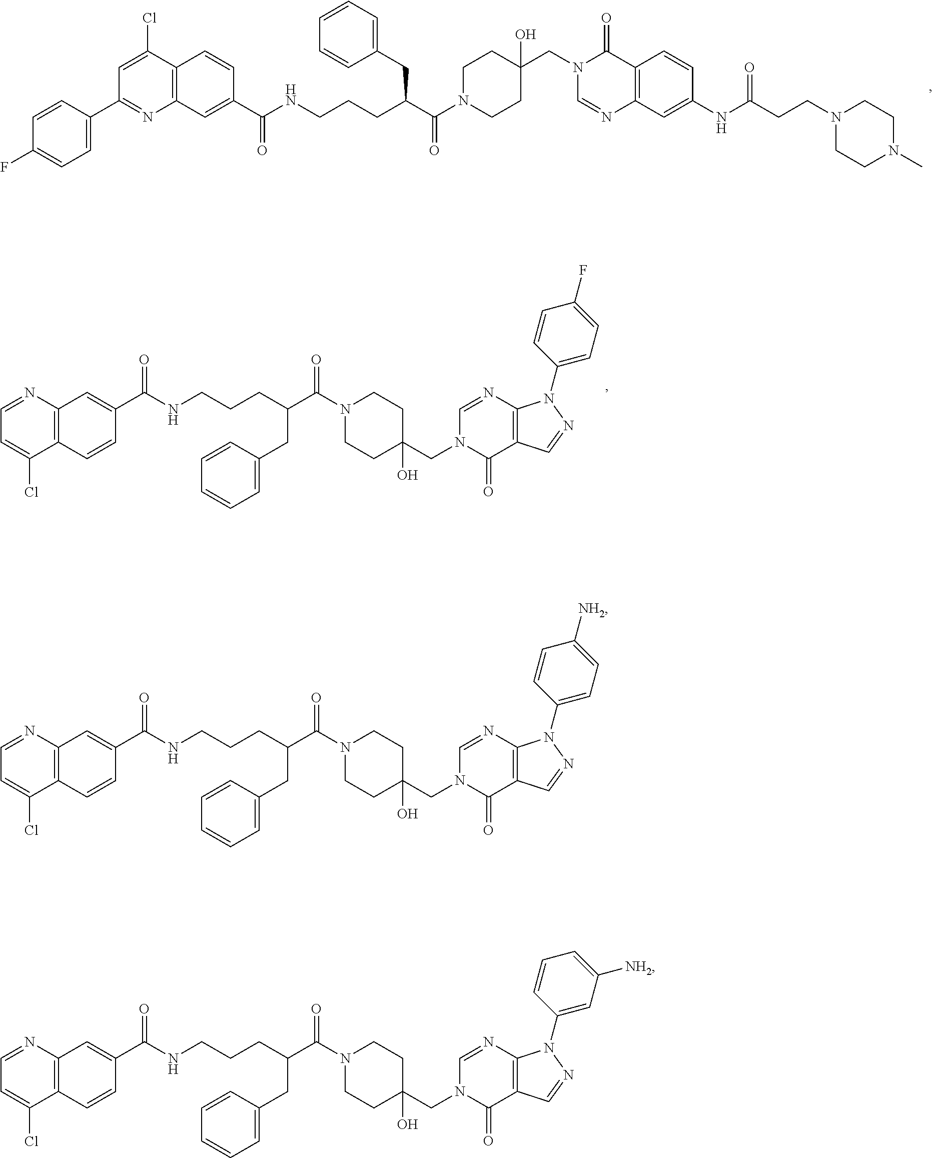 [SMILES: CN1CCN(CCC(=O)Nc2ccc3c(=O)n(CC4(O)CCN(C(=O)[C@@H](CCCNC(=O)c5ccc6c(Cl)cc(-c7ccc(F)cc7)nc6c5)Cc5ccccc5)CC4)cnc3c2)CC1 ; O=C(NCCCC(Cc1ccccc1)C(=O)N1CCC(O)(Cn2cnc3c(cnn3-c3ccc(F)cc3)c2=O)CC1)c1ccc2c(Cl)ccnc2c1 ; Nc1ccc(-n2ncc3c(=O)n(CC4(O)CCN(C(=O)C(CCCNC(=O)c5ccc6c(Cl)ccnc6c5)Cc5ccccc5)CC4)cnc32)cc1 ; Nc1cccc(-n2ncc3c(=O)n(CC4(O)CCN(C(=O)C(CCCNC(=O)c5ccc6c(Cl)ccnc6c5)Cc5ccccc5)CC4)cnc32)c1]

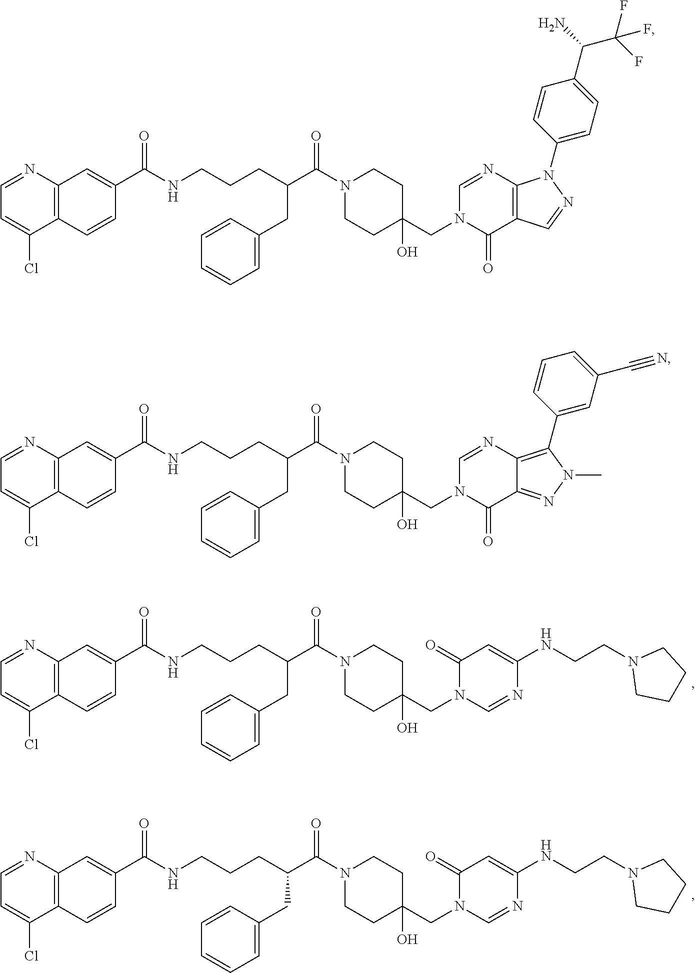 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F ; Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1 ; O=C(NCCCC(Cc1ccccc1)C(=O)N1CCC(O)(Cn2cnc(NCCN3CCCC3)cc2=O)CC1)c1ccc2c(Cl)ccnc2c1 ; O=C(NCCC[C@@H](Cc1ccccc1)C(=O)N1CCC(O)(Cn2cnc(NCCN3CCCC3)cc2=O)CC1)c1ccc2c(Cl)ccnc2c1]

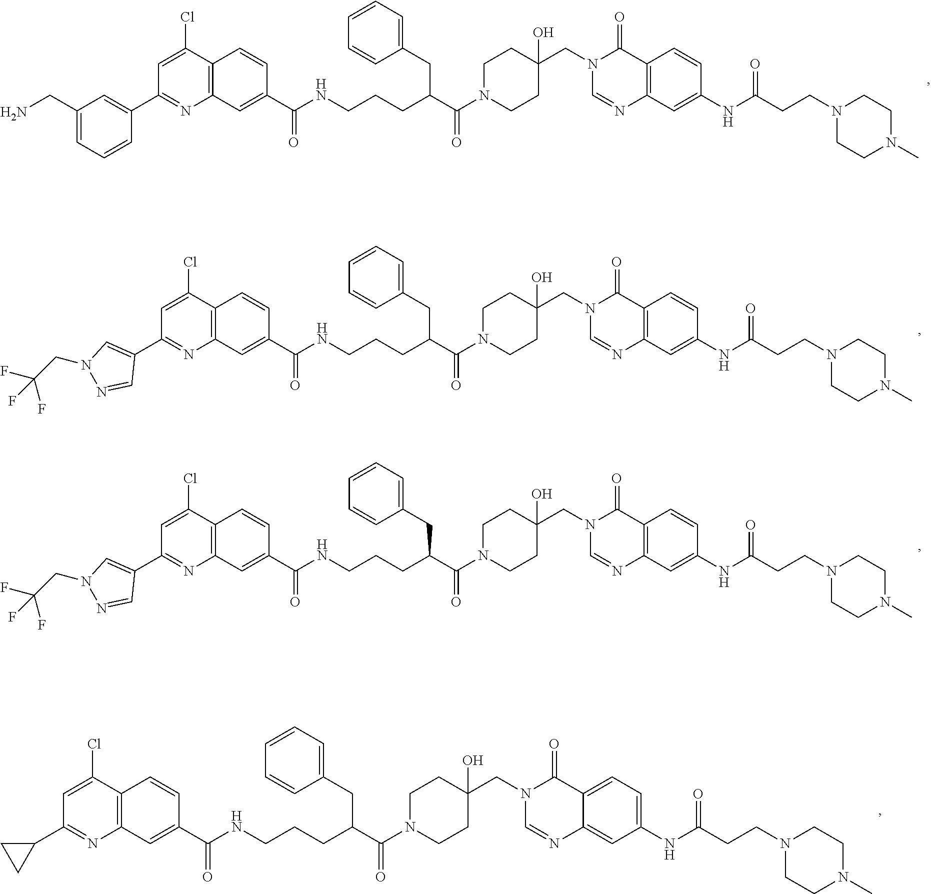 [SMILES: CN1CCN(CCC(=O)Nc2ccc3c(=O)n(CC4(O)CCN(C(=O)C(CCCNC(=O)c5ccc6c(Cl)cc(-c7cccc(CN)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7cnn(CC(F)(F)F)c7)nc6c5)Cc5ccccc5)CC4)cnc3c2)CC1 ; CN1CCN(CCC(=O)Nc2ccc3c(=O)n(CC4(O)CCN(C(=O)[C@@H](CCCNC(=O)c5ccc6c(Cl)cc(-c7cnn(CC(F)(F)F)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(C7CC7)nc6c5)Cc5ccccc5)CC4)cnc3c2)CC1]

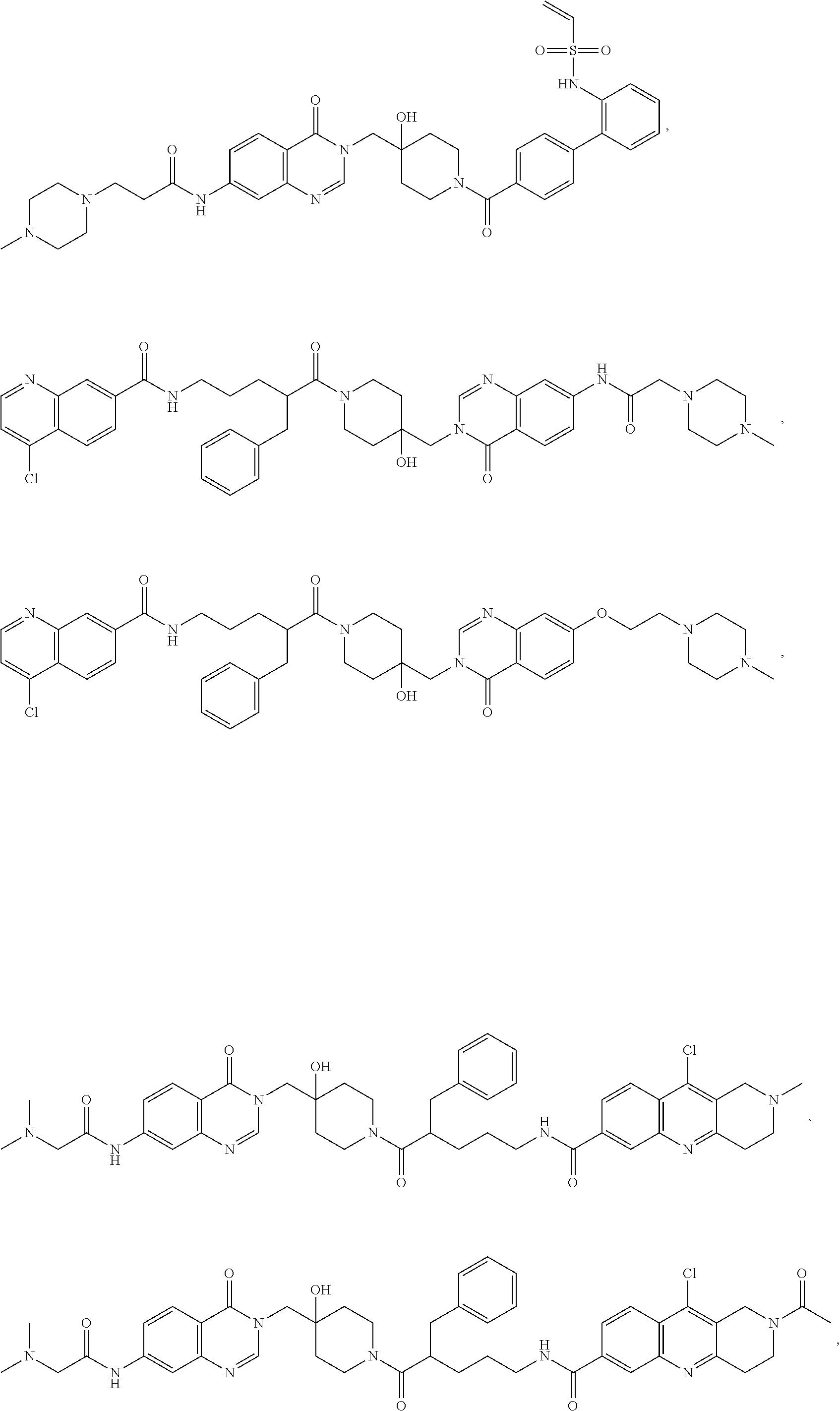

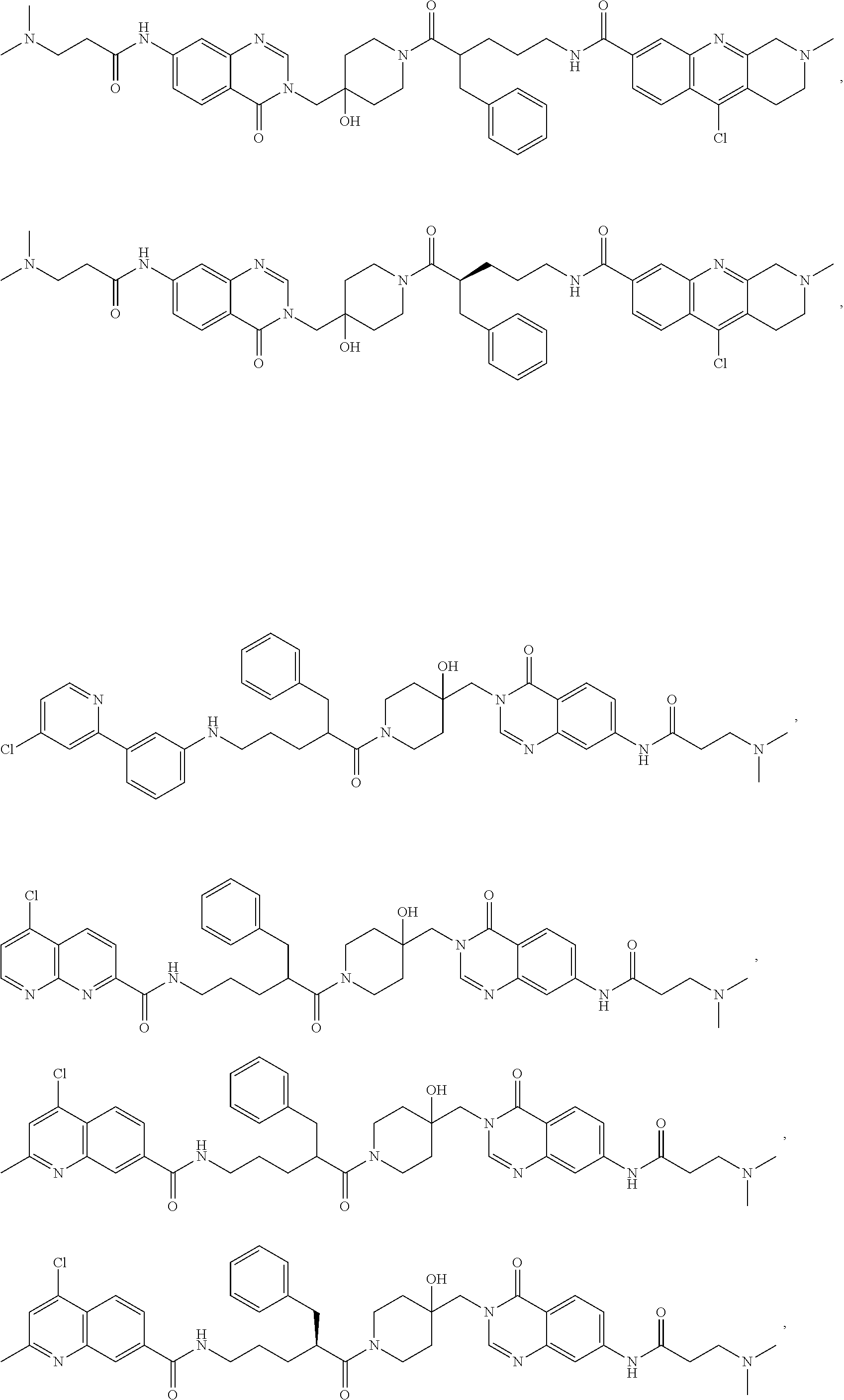

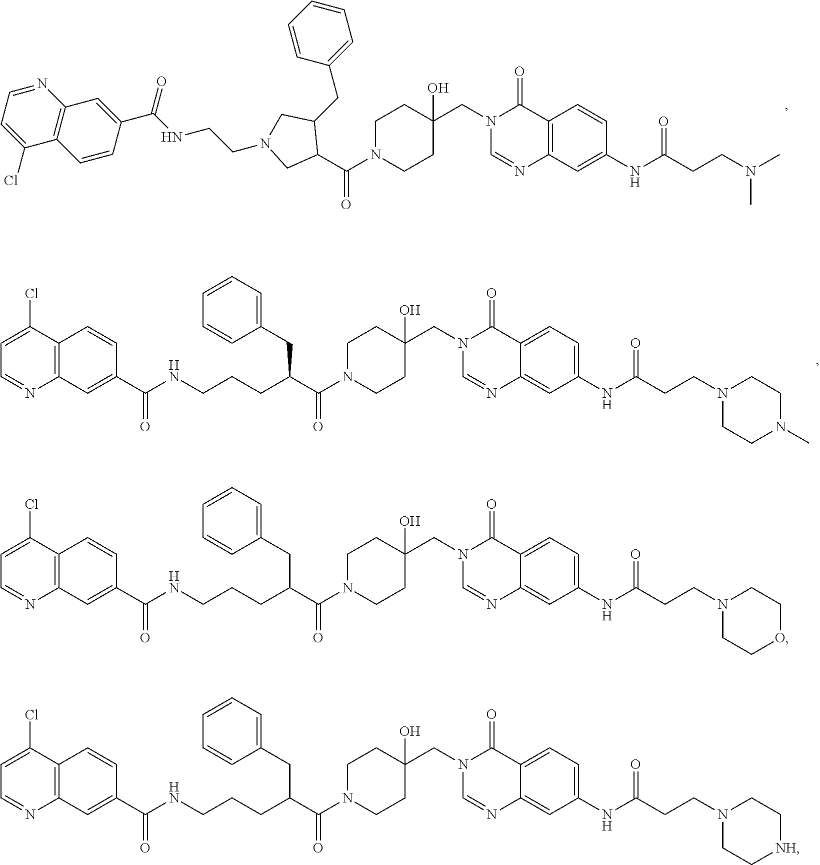 [SMILES: CN(C)CCC(=O)Nc1ccc2c(=O)n(CC3(O)CCN(C(=O)C4CN(CCNC(=O)c5ccc6c(Cl)ccnc6c5)CC4Cc4ccccc4)CC3)cnc2c1 ; CN1CCN(CCC(=O)Nc2ccc3c(=O)n(CC4(O)CCN(C(=O)[C@@H](CCCNC(=O)c5ccc6c(Cl)ccnc6c5)Cc5ccccc5)CC4)cnc3c2)CC1 ; O=C(CCN1CCOCC1)Nc1ccc2c(=O)n(CC3(O)CCN(C(=O)C(CCCNC(=O)c4ccc5c(Cl)ccnc5c4)Cc4ccccc4)CC3)cnc2c1 ; O=C(CCN1CCNCC1)Nc1ccc2c(=O)n(CC3(O)CCN(C(=O)C(CCCNC(=O)c4ccc5c(Cl)ccnc5c4)Cc4ccccc4)CC3)cnc2c1]

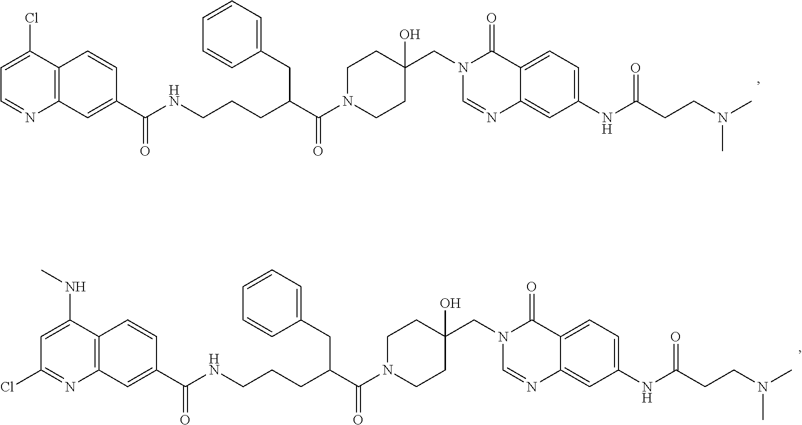 [SMILES: CN(C)CCC(=O)Nc1ccc2c(=O)n(CC3(O)CCN(C(=O)C(CCCNC(=O)c4ccc5c(Cl)ccnc5c4)Cc4ccccc4)CC3)cnc2c1 ; CNc1cc(Cl)nc2cc(C(=O)NCCCC(Cc3ccccc3)C(=O)N3CCC(O)(Cn4cnc5cc(NC(=O)CCN(C)C)ccc5c4=O)CC3)ccc12]

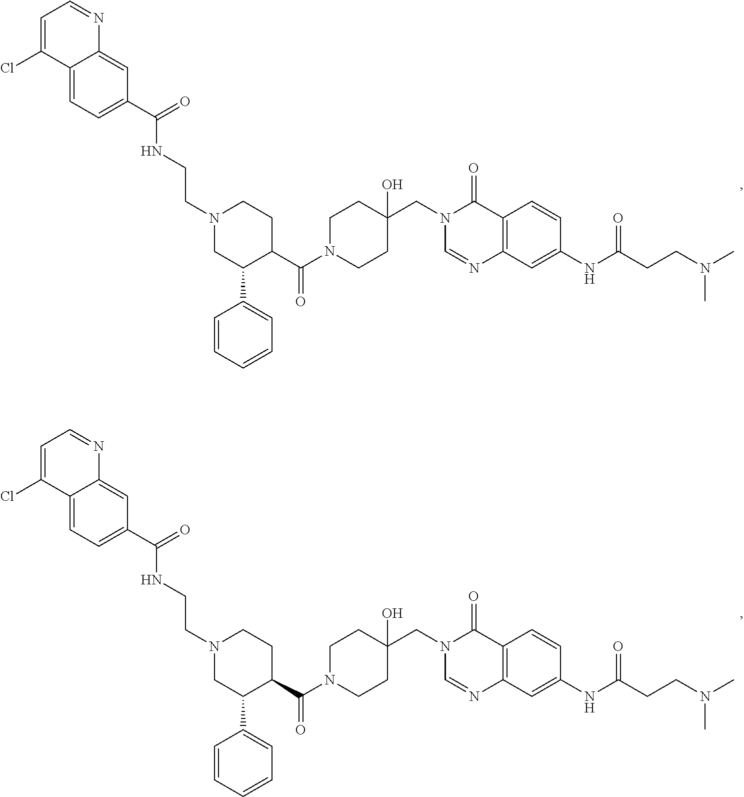 [SMILES: CN(C)CCC(=O)Nc1ccc2c(=O)n(CC3(O)CCN(C(=O)C4CCN(CCNC(=O)c5ccc6c(Cl)ccnc6c5)C[C@H]4c4ccccc4)CC3)cnc2c1 ; CN(C)CCC(=O)Nc1ccc2c(=O)n(CC3(O)CCN(C(=O)[C@@H]4CCN(CCNC(=O)c5ccc6c(Cl)ccnc6c5)C[C@H]4c4ccccc4)CC3)cnc2c1]

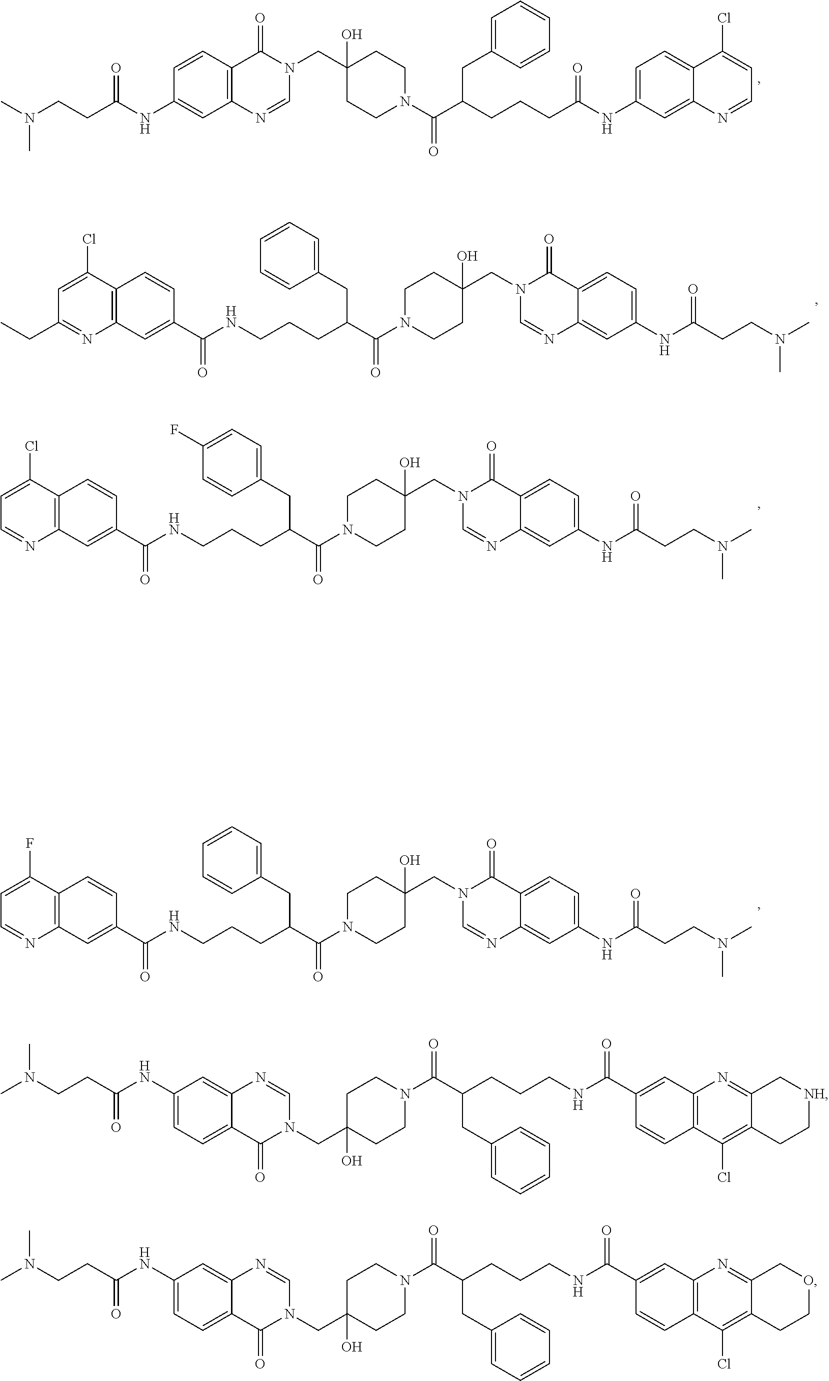

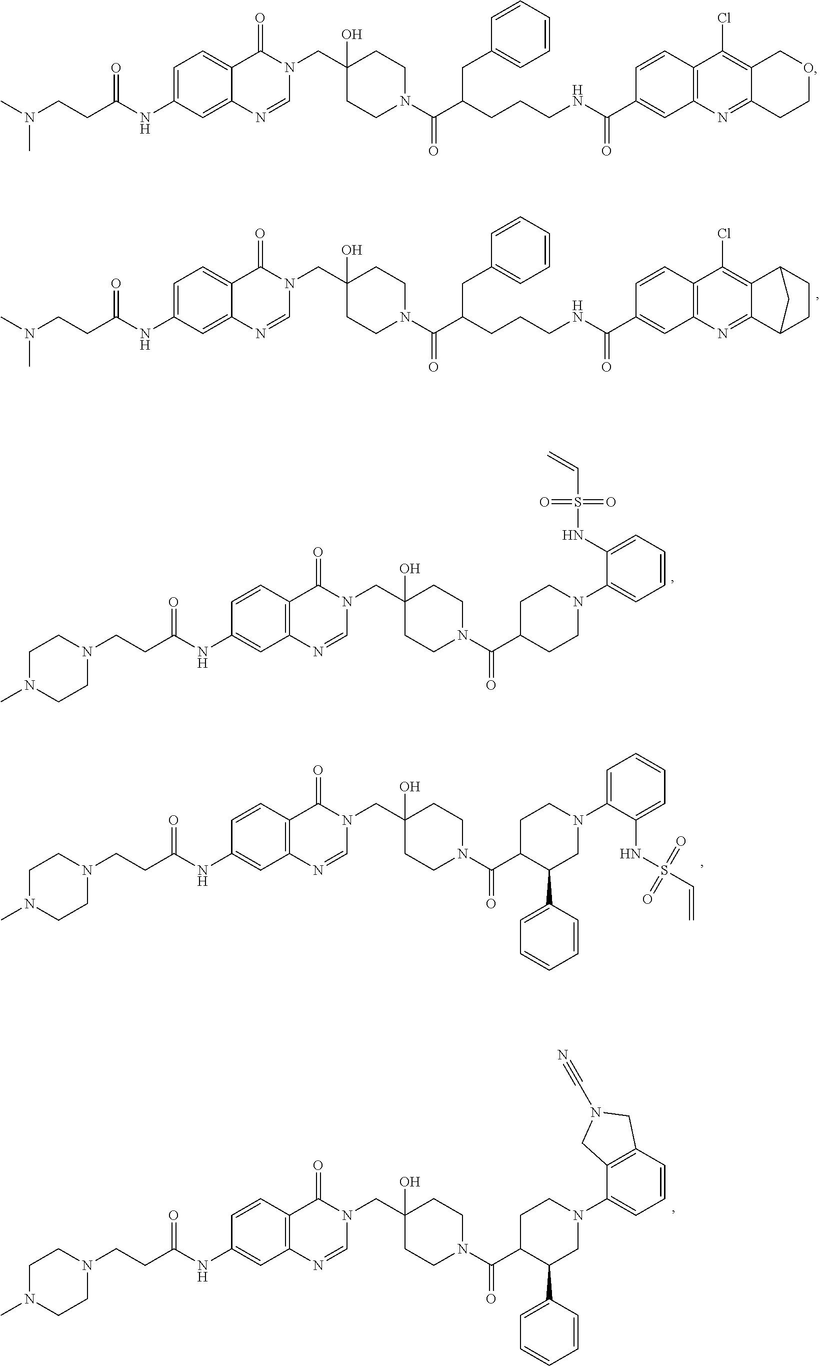

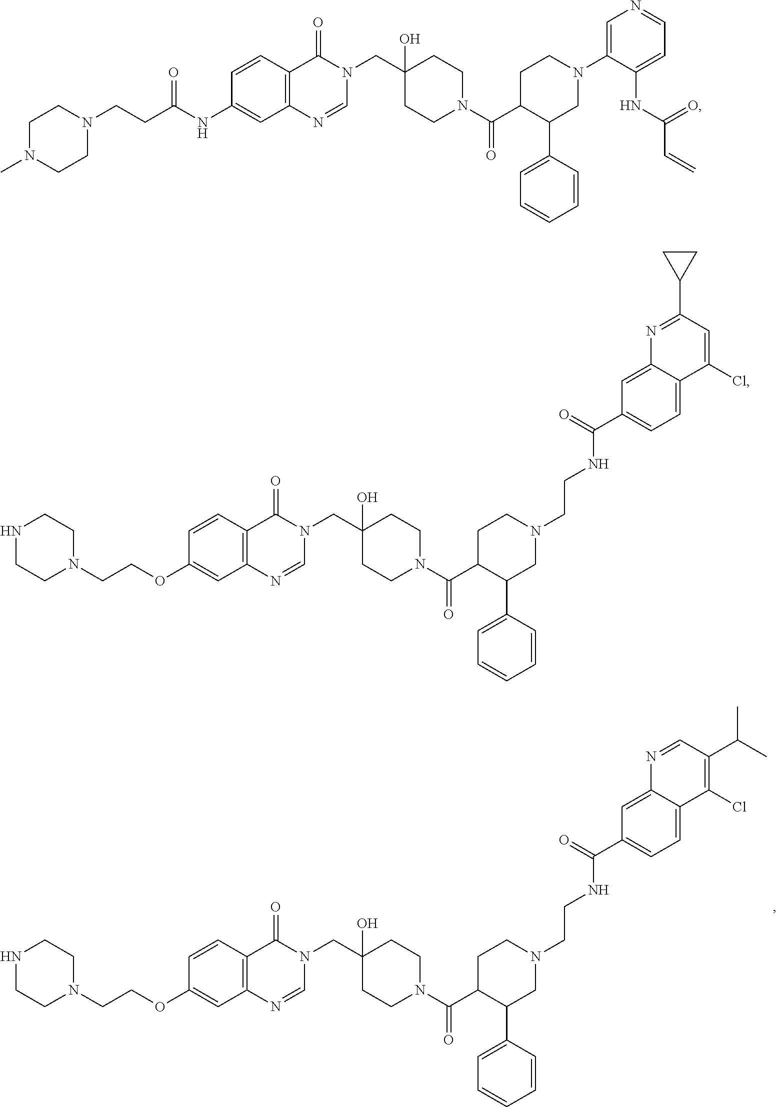 [SMILES: C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1 ; O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1 ; CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl]

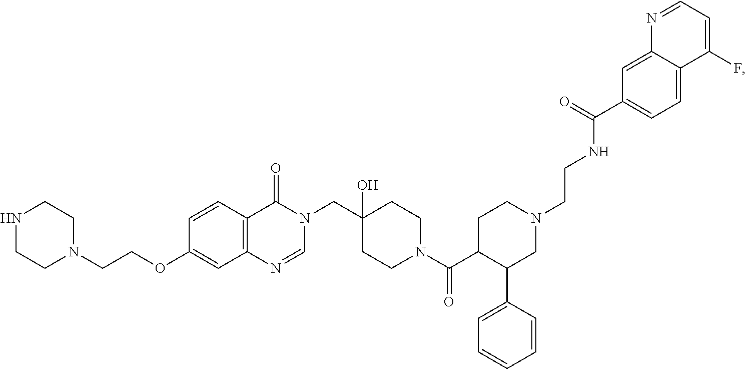 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1]

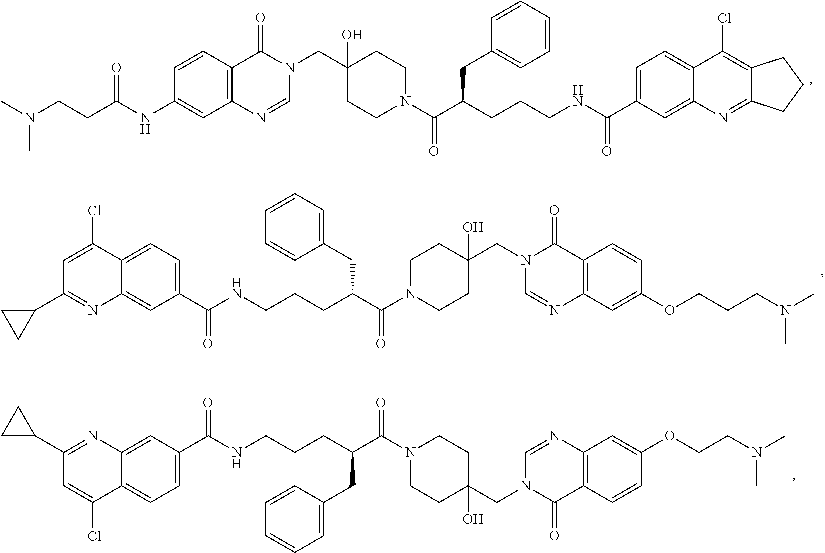 [SMILES: CN(C)CCC(=O)Nc1ccc2c(=O)n(CC3(O)CCN(C(=O)[C@H](CCCNC(=O)c4ccc5c(Cl)c6c(nc5c4)CCC6)Cc4ccccc4)CC3)cnc2c1 ; CN(C)CCCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1 ; CN(C)CCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1]

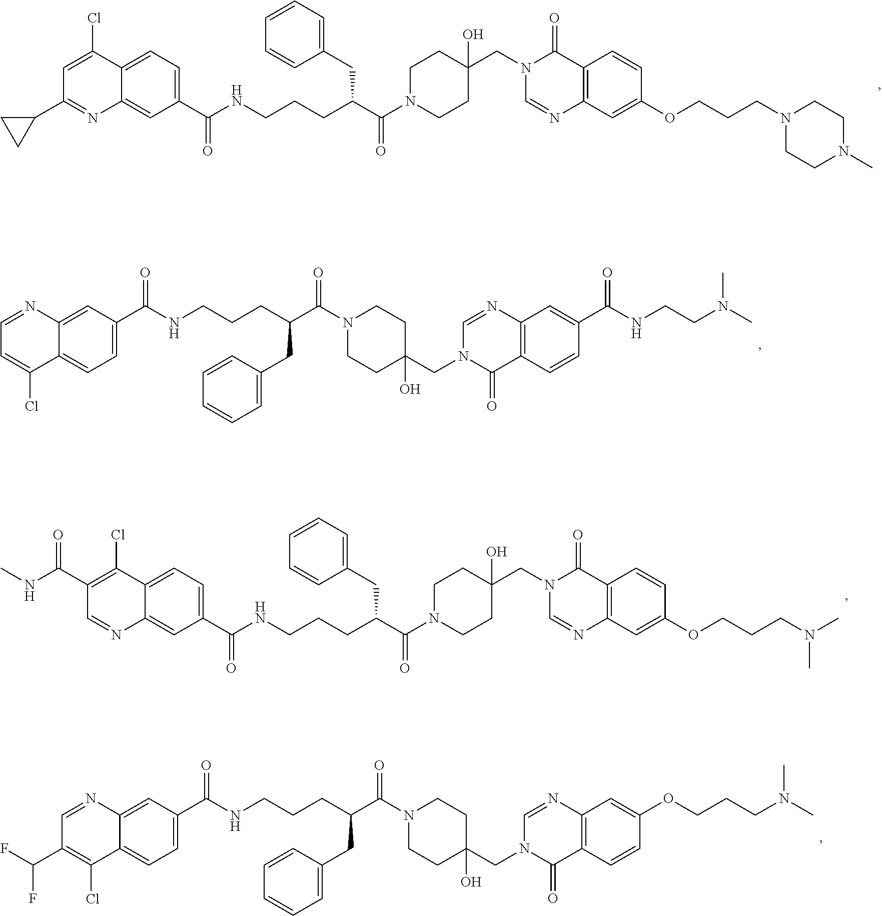

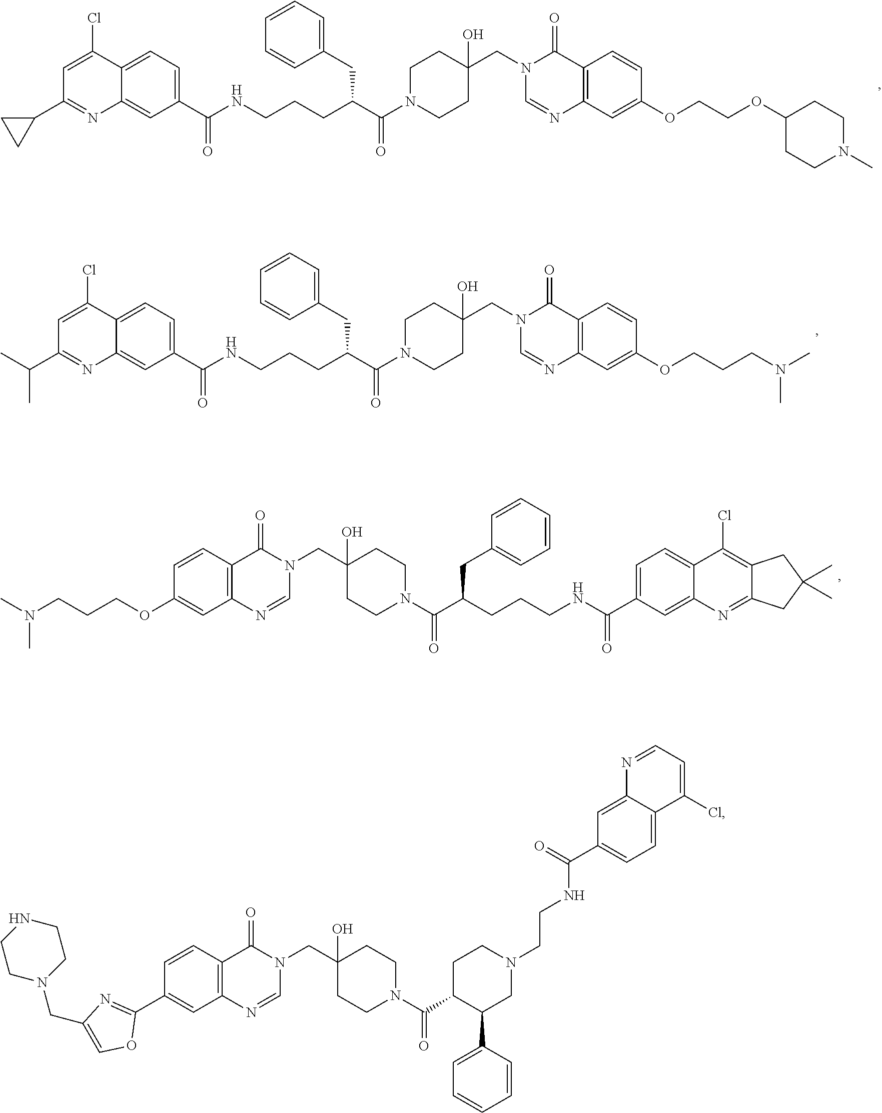

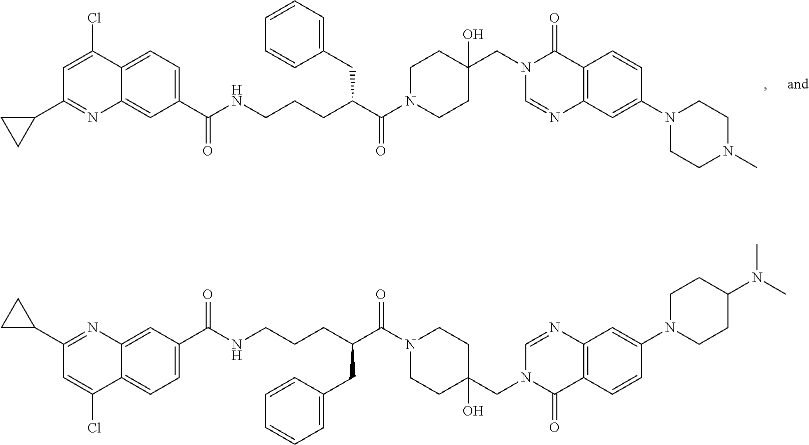 [SMILES: CN1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1 ; CN(C)C1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1]

or a pharmaceutically acceptable salt thereof.

In some embodiments, the present disclosure relates to a pharmaceutical composition comprising a compound of formula (I), such as a compound selected from compounds 4-144, and a pharmaceutically acceptable carrier.

In some embodiments, the present disclosure relates to a method of treating a disease or disorder modulated by USP7, comprising administering to a subject in need thereof a compound of formula (I), such as a compound selected from compounds 4-144, or a pharmaceutical composition comprising a compound of formula (I), such as a compound selected from compounds 4-144.

In some embodiments, the present disclosure relates to a method of inhibiting USP7, comprising administering to a subject in need thereof a compound of formula (I), such as a compound selected from compounds 4-144, or a pharmaceutical composition comprising a compound of formula (I), such as a compound selected from compounds 4-144. In some embodiments, the disease or disorder associated with inhibition of USP7 is cancer and metastasis, neurodegenerative diseases, immunological disorders, diabetes, bone and joint diseases, osteoporosis, arthritis inflammatory disorders, cardiovascular diseases, ischemic diseases, viral infections and diseases, viral infectivity and/or latency, and bacterial infections and diseases.

In some embodiments, the present disclosure relates to a method of treating cancer, comprising administering to a subject in need thereof a compound of formula (I), such as a compound selected from compounds 4-144, or a pharmaceutical composition comprising a compound of formula (I), such as a compound selected from compounds 4-144. In some embodiments, the cancer is liposarcoma, neuroblastoma, glioblastoma, breast cancer, bladder cancer, glioma, adrenocortical cancer, multiple myeloma, colorectal cancer, colon cancer, prostate cancer, non-small cell lung cancer, Human Papilloma Virus-associated cervical cancer, oropharyngeal cancer, penis cancer, ovarian cancer, anal cancer, thyroid cancer, vaginal cancer, Epstein-Barr Virus-associated nasopharyngeal carcinoma, gastric cancer, rectal cancer, thyroid cancer, Hodgkin lymphoma, diffuse large B-cell lymphoma, and Ewing sarcoma. In some embodiments, the cancer is neuroblastoma, multiple myeloma, breast cancer, glioma, colon cancer, prostate cancer, or ovarian cancer. In some embodiments, the cancer is multiple myeloma. In some embodiments, the cancer is Ewing sarcoma.

In some embodiments, the present disclosure relates to a method of inhibiting USP7, wherein thereof a compound of formula (I), such as a compound selected from compounds 4-144, forms a covalent bond with USP7. In some embodiments, the covalent bond forms with a cysteine residue of USP7.

In some embodiments, the present disclosure relates to a use of thereof a compound of formula (I), such as a compound selected from compounds 4-144, for the manufacture of a medicament for treating a disease modulated by USP7.

In some embodiments, the present disclosure relates to thereof a compound of formula (I), such as a compound selected from compounds 4-144, for use in treating a disease modulated by USP7.

### Methods of Use

Ubiquitin is a 76-residue protein that is dynamically conjugated to proteins via an isopeptide bond. Canonically, ubiquitin's C-terminal glycine is linked to a substrate lysine side chain, and ubiquitin can also be conjugated to substrates via cysteine, serine and threonine side chains as well as the N-terminal amine. McDowell, G. S. & Philpott, A. Non-canonical ubiquitylation: Mechanisms and consequences. Int. J. Biochem. Cell Biol. 45, 1833-1842 (2013). Ubiquitin itself possesses 7 lysine side chains, and there are naturally occurring linear or mixed chains of ubiquitin conjugated through these lysine side chains or the N-terminal methionine residue. Ubiquitin conjugation is achieved through the concerted action of ubiquitin-activating (E1), conjugating (E2), and ligating (E3) enzymes, and it can be reversed by deubiquitinating enzymes (DUBs). Mono-ubiquitin tags or ubiquitin chains of different topologies mediate protein conformational changes and binding to numerous scaffolding and adaptor proteins, and ubiquitination plays a key role in many cellular processes including proteasomal degradation (Nandi, D., et al., The Ubiquitin-Proteasome System. J Biosci 31, 137-155 (2016)), membrane trafficking (Hurley, J. H. & Stenmark, H. Molecular Mechanisms of Ubiquitin-Dependent Membrane Traffic. Annu. Rev. Biophys. 40, 119-142 (2011)), chromatin dynamics (Shilatifard, A. Chromatin Modifications by Methylation and Ubiquitination: Implications in the Regulation of Gene Expression. Annu. Rev. Biochem. 75, 243-269 (2006)), and DNA repair (Jackson, S. P. & Durocher, D. Review Regulation of DNA Damage Responses by Ubiquitin and SUMO. Mol. Cell 49, 795-807 (2013)). Ubiquitin signaling is also implicated in numerous disease settings, including cancer (Senft, D., Qi, J. & Ronai, Z. A. Ubiquitin ligases in oncogenic transformation and cancer therapy. Nat. Rev. Cancer 18, 69-88 (2018); Pinto-Fernandez, A. & Kessler, B. M. DUBbing cancer: Deubiquitylating enzymes involved in epigenetics, DNA damage and the cell cycle as therapeutic targets. Front. Genet. 7, 1-13 (2016)), infection (Isaacson, M. K. & Ploegh, H. L. Ubiquitination, Ubiquitin-like Modifiers, and Deubiquitination in Viral Infection. Cell Host Microbe 5, 559-570 (2009)), and neurodegeneration (Ciechanover, A. & Brundin, P. The ubiquitin proteasome system in neurodegenerative diseases: sometimes the chicken, sometimes the egg. Neuron 40, 427-446 The ubiquitin proteasome system in neurodeg (2003)). In particular, the ubiquitin-proteasome system (UPS) has become a target of interest in oncology, as both proteasome inhibitors and bivalent substrate-E3 ligands have been approved as targeted cancer therapies (Manasanch, E. E. & Orlowski, R. Z. Proteasome inhibitors in cancer therapy. Nat. Rev. Clin. Oncol. 14, 417-433 (2017); Bartlett, J. B., et al. The evolution of thalidomide and its IMiD derivatives as anticancer agents. Nat. Rev. Cancer 4, 314-322 (2004)). There are currently no DUB inhibitors in the clinic, a reality driven in part by a dearth of high quality probe compounds for addressing both explorations of fundamental DUB biology and target validation in preclinical disease models.

There are ˜100 human DUBs belonging to seven distinct families, six of which are cysteine proteases (ubiquitin-specific protease [USP], ubiquitin C-terminal hydrolase [UCH], ovarian tumor protease [OTU], Josephin, Mindy, and ZUFSP), and one of which is a family of zinc metalloproteases (JAB/MPN/MOV34 [JAMM/MPN]). Several high quality probes targeting USP7 recently have been developed. These probes share the characteristics of single- or double-digit nM potency against USP7, co-structural confirmation of USP7 catalytic domain binding, and activity profiling verifying selectivity against 40+ DUBs. Lamberto, I. et al. Structure-Guided Development of a Potent and Selective Non-covalent Active-Site Inhibitor of USP7. Cell Chem. Biol. 24, 1490-1500 (2017); Kategaya, L. et al. USP7 small-molecule inhibitors interfere with ubiquitin binding. Nature 550, 534-538 (2017); Turnbull, A. P. et al. Molecular basis of USP7 inhibition by selective small-molecule inhibitors. Nature 550, 481-486 (2017); and Gavory, G. et al. Discovery and characterization of highly potent and selective allosteric USP7 inhibitors. 7, (2017). Collectively, this work represented a sea change in thinking about the druggability of USP7 and DUBs more broadly; prior to 2017, no USP:small molecule co-crystal structures had been published in the Protein Data Bank (PDB), and DUB profiling reported by research had consistently found that previously reported DUB inhibitors typically had weak (≥1 μM) affinity and lacked a high degree of selectivity among DUBs. Ritorto, M. S. et al. Screening of DUB activity and specificity by MALDI-TOF mass spectrometry. Nat. Commun. 5, 4763 (2014).

USP7 is one of the most widely studied DUBs, and it has been associated with multiple substrates, cellular pathways, and disease states. USP7 was first discovered as an interacting partner and stabilizer of the Herpesvirus E3 ligase ICP0. Everett, R. D. et al. A novel ubiquitin-specific protease is dynamically associated with the PML nuclear domain and binds to a herpesvirus regulatory protein. 16, 1519-1530 (1997). Since then, USP7 has also been reported to interact with and regulate numerous mammalian E3 ligases, including MDM2 (Li, M., et al. A dynamic role of HAUSP in the p53-Mdm2 pathway. Mol. Cell 13, 879-886 (2004)), UHRF1 (Ma, H. et al. M phase phosphorylation of the epigenetic regulator UHRF1 regulates its physical association with the deubiquitylase USP7 and stability. Proc. Natl. Acad. Sci. 109, 4828-4833 (2012)), TRIM27 (Zaman, M. M.-U. et al. Ubiquitination-Deubiquitination by the TRIM27-USP7 Complex Regulates Tumor Necrosis Factor Alpha-Induced Apoptosis. Mol. Cell. Biol. 33, 4971-4984 (2013)), RING1B (de Bie, P. et al. Regulation of the Polycomb protein RING1B ubiquitination by USP7. Biochem. Biophys. Res. Commun. 400, 389-395 (2010)), RAD18 (Zlatanou, A. et al. USP7 is essential for maintaining Rad18 stability and DNA damage tolerance. 35, 965-976 (2015)), RNF220 (Ma, P. et al. The Ubiquitin Ligase RNF220 Enhances Canonical Wnt Signaling through USP7-Mediated Deubiquitination of -Catenin. Mol. Cell. Biol. 34, 4355-4366 (2014)), MARCH7 (Nathan, J. A. et al. The ubiquitin E3 ligase MARCH7 is differentially regulated by the deubiquitylating enzymes USP7 and USP9X. Traffic 9, 1130-1145 (2008)), RNF168 (Zhu, Q., Sharma, N., He, J., Wani, G. & Wani, A. A. USP7 deubiquitinase promotes ubiquitin-dependent DNA damage signaling by stabilizing RNF168. Cell Cycle 14, 1413-1425 (2015)), and RNF169 (An, L. et al. Dual-utility NLS drives RNF169-dependent DNA damage responses. Proc. Natl. Acad. Sci. 114, E2872-E2881 (2017)). In addition, USP7 has been found in a binary complex with both GMPS and UVSSA, and USP7 binding appears to be essential for these proteins' cellular function. Van Der Knaap, J. A. et al. GMP synthetase stimulates histone H2B deubiquitylation by the epigenetic silencer USP7. Mol. Cell 17, 695-707 (2005); Schwertman, P. et al. UV-sensitive syndrome protein UVSSA recruits USP7 to regulate transcription-coupled repair. Nat. Genet. 44, 598-602 (2012).

Of all these potential substrates, USP7's interaction with MDM2 has garnered the most interest from a mechanistic and therapeutic standpoint. USP7 binds both MDM2 and p53 through its TRAF domain and has been shown to have DUB activity toward both of these proteins. There is an emerging hypothesis that USP7 acts as a molecular switch, where it deubiquitinates and stabilizes MDM2 during normal cell growth but will change its preferred substrate to p53 in the presence of cellular stress signals. Brazhnik, P. & Kohn, K. W. HAUSP-regulated switch from auto- to p53 ubiquitination by Mdm2 (in silico discovery). Math. Biosci. 210, 60-77 (2007); Kim, R. Q. & Sixma, T. K. Regulation of USP7: A high incidence of E3 complexes. J. Mol. Biol. 429, 3395-3408 (2017). Given the key role of p53 in tumor suppression, USP7 has been proposed as a therapeutic target in TP53-WT tumors, with a putative mechanism-of-action that involves increasing p53 protein levels, similar to the effects of the MDM2-p53 interaction inhibitor RG-7388 and the MDM2/MDM4 dual inhibitor ATSP-7041, which are both currently under clinical investigation. Ding, Q. et al. Discovery of RG7388, a potent and selective p53-MDM2 inhibitor in clinical development. J Med. Chem. 56, 5979-5983 (2013); Chang, Y. S. et al. Stapled α-helical peptide drug development: A potent dual inhibitor of MDM2 and MDMX for p53-dependent cancer therapy. Proc. Natl. Acad. Sci. 110, E3445-E3454 (2013). However, given that USP7 targets multiple substrates, there is an open debate about the relative importance of p53 mutational status in predicting response to USP7 inhibition. Several prior studies on non-selective USP7 inhibitors have indicated that USP7 inhibitors are effective against both p53 WT and mutant disease. Chauhan, D. et al. Article A Small Molecule Inhibitor of Ubiquitin-Specific Protease-7 Induces Apoptosis in Multiple Myeloma Cells and Overcomes Bortezomib Resistance. Cancer Cell 22, 345-358 (2012); Wang, M. et al. The USP7 Inhibitor P5091 Induces Cell Death in Ovarian Cancers with Different P53 Status. Cell. Physiol. Biochem. 43, 1755-1766 (2018). These results were supported in studies using Genentech's DUB-selective USP7 inhibitor GNE-6640, which did not produce significantly different responses in TP53-WT or mutant cell lines when screened in a 181-cell panel. Kategaya, L. et al. USP7 small-molecule inhibitors interfere with ubiquitin binding. Nature 550, 534-538 (2017). On the other hand, it has been found that in the case of the selective USP7 inhibitor compound 42, TP53 status is a key predictor of response in Ewing sarcoma and other cancer cell types. Roti, G. et al., J Exp. Med., 215, 197-216 (2018).

One of the major missing pieces in previous reports of selective USP7 inhibitors was the spectrum of off targets outside of the DUB family. A well annotated off target profile would help clarify whether the p53-independent effects of a USP7 inhibitor are due to other USP7 substrates or other compound targets. Based on the structure of compound 42 bound to USP7's catalytic domain, a rational synthesis of an irreversible, affinity-taggable analog was designed that would be sufficient for proteome-wide profiling experiments and follow-up studies on USP7/p53 biology.

USP7 also alters the level of the p16INK4a tumor suppressor through Bmi1/Mel18 stabilization. Maertens et al., Embo J. 29, 2553-2565 (2010). Additional proteins involved in genomic integrity/regulation such as the DNMT1 DNA methylase and the Claspin adaptor are also stabilized by USP7. Du et al., Science Signaling, 3(146):ra80 (2010); Faustrup et al., J. Cell Biol., 184(1):13-9 (2009). Importantly, the abundance of USP7 and DNMT1, a protein involved in maintaining epigenetic methylation required to silence genes involved in development and cancer, correlates in human colon cancer (Du et al., 2010). USP7 has also been shown in human cells to deubiquitinate the well-known tumor suppressor gene PTEN, which provokes its nuclear export and hence its inactivation. Song et al., Nature, 455(7214), 813-7 (2008). More importantly, USP7 overexpression was reported for the first time in prostate cancer and this overexpression was directly associated with tumour aggressiveness (Song et al., 2008).

Recently, several epigenetic modifiers, including the methyltransferase PHF8 (Wang et al., 2016a), demethylase DNMT1 (Du et al., 2010, Felle et al., Nucleic Acids Res, 39, 8355-65, 2011, Qin et al., J Cell Biochem, 112, 439-44, 2011), and acetyltransferase Tip60, (Dar et al., Mol Cell Biol, 33, 3309-20, 2013), as well as H2B itself (van der Knaap et al., Mol Cell, 17, 695-707, 2005) have been identified as direct targets of USP7. Other notable targets of USP7 include the transcription factors FOXP3, which in Treg cells links this DUB enzyme to immune response (van Loosdregt et al., Immunity, 39, 259-71, 2013), and N-Myc, which is stabilized in neuroblastoma cells. Tavana et al., Nat Med, 22, 1180-1186, 2016. Consistent with its regulation of diverse substrates and biological processes USP7 has emerged as a drug target in a wide range of malignancies including multiple myeloma (Chauhan et al., Cancer Cell, 22, 345-58, 2012), breast cancer (Wang et al., 2016a), neuroblastoma (Tavana et al., 2016), glioma (Cheng et al., Oncol Rep, 29, 1730-6, 2013), and ovarian cancer (Zhang et al., Tohoku J Exp Med, 239, 165-75, 2016). USP7 has also been shown in human cells to deubiquitinate FOXO4, which provokes its nuclear export and hence its inactivation; consequently the oncogenic PI3K/PKB signaling pathway was activated (van der Horst et al., Nat Cell Biol. 2006, 8, 1064-1073) Finally, USP7 plays an important role in p53-mediated cellular responses to various types of stress, such as DNA damage and oxidative stress (Marchenko et al., Embo J. 2007 26, 923-934, Meulmeester et al., Mol Cell 2005, 18, 565-576., van der Horst et al., Nat Cell Biol. 2006, 8, 1064-1073).

Multiple myeloma (MM) is an incurable hematological malignancy characterized by the accumulation of abnormal plasma cells in the bone marrow, which impede production of normal blood cells. The average survival of MM patients has improved in recent years as a result of the introduction of proteasome inhibitors and immunomodulatory agents into treatment regimens but is still quite poor at only 5 years. The proteasome inhibitor bortezomib validates the ubiquitin proteasome system as a therapeutic target for MM drug development. USP7 is a therapeutic target in MM due to its role in the degradation of p53. USP7 is highly expressed in MM patient tumor cells and MM cell lines versus normal bone marrow cells. Mutations or deletions in TP53 are late events in MM suggesting that increasing p53 via pharmacological inhibition of USP7 could be an effective therapeutic strategy for this malignancy.

Ewing sarcoma is a rare type of cancer that occurs in bones or in the soft tissue around the bones. Ewing sarcoma is more common in teenagers and young adults. The current standard of care for Ewing sarcoma is chemotherapy, radiation, and surgery.

Disclosed herein are methods for treating and preventing diseases and conditions that benefit from the modulation of USP7, comprising administering to a subject in need thereof any one of the compounds disclosed herein, or a pharmaceutically acceptable salt thereof.

Disclosed herein are methods for treating and preventing diseases and conditions that benefit from the inhibition of USP7, comprising administering to a subject in need thereof any one of the compounds disclosed herein, or a pharmaceutically acceptable salt thereof.

Disclosed herein are methods for inhibiting USP7, comprising administering to a subject in need thereof any one of the compounds disclosed herein, or a pharmaceutically acceptable salt thereof.

In some embodiments, disclosed herein are methods of treating a disease or disorder modulated by USP7, comprising administering to a subject in need thereof any one of the compounds disclosed herein, or a pharmaceutically acceptable salt thereof. In some embodiments, disclosed herein are methods of preventing a disease or a disorder modulated by USP7 comprising administering to a subject in need thereof any one of the compounds disclosed herein, or a pharmaceutically acceptable salt thereof. In some embodiments, the modulation of USP7 involves inhibiting USP7.

In some embodiments, the disease or disorder is selected from cancer and metastasis, neurodegenerative diseases, immunological disorders, diabetes, bone and joint diseases, osteoporosis, arthritis inflammatory disorders, cardiovascular diseases, ischemic diseases, viral infections and diseases, viral infectivity and/or latency, and bacterial infections and diseases.

Disclosed herein is the use of an inhibitor of USP7 for the preparation of a medicament for treating or preventing a disease or condition modulated by USP7, wherein the medicament comprises any one of the compounds disclosed herein, or a pharmaceutically acceptable salt thereof.

Disclosed herein are any one of the disclosed compounds, or a pharmaceutically acceptable salt thereof, for use in treating a disease or condition modulated by USP7.

Disclosed herein are methods of treating cancer comprising administering to a subject in need thereof any one of the compounds disclosed herein, or a pharmaceutically acceptable salt thereof.

Disclosed herein are methods of inhibiting USP7, wherein any one of the compounds disclosed herein, or a pharmaceutically acceptable salt thereof, forms a covalent bond with USP7. In some embodiments, the covalent bond forms with a cysteine residue of USP7. In some embodiments, the cysteine residue of USP7 is cysteine 223 (C223).

In some embodiments of the methods and uses disclosed herein, the modulation of USP7 involves inhibiting USP7. In some embodiments, inhibition of USP7 is irreversible. In some embodiments, inhibiting USP7 is a novel treatment for a disease or condition.

In some embodiments, exemplary cancers include, but are not limited to, p53 WT cancers.

In some embodiments, exemplary cancers include, but are not limited to, solid tumors.

In some embodiments, exemplary cancers include, but are not limited to, liposarcoma, neuroblastoma, glioblastoma, breast cancer, bladder cancer, glioma, adrenocortical cancer, multiple myeloma, colorectal cancer, colon cancer, prostate cancer, non-small cell lung cancer, Human Papilloma Virus-associated cervical cancer, oropharyngeal cancer, penis cancer, ovarian cancer, anal cancer, thyroid cancer, vaginal cancer, Epstein-Barr Virus-associated nasopharyngeal carcinoma, gastric cancer, rectal cancer, thyroid cancer, Hodgkin lymphoma, diffuse large B-cell lymphoma, and Ewing sarcoma.

In some embodiments, the cancers are selected from neuroblastoma, multiple myeloma, breast cancer, glioma, colon cancer, prostate cancer, and ovarian cancer. In some embodiments, the cancer is neuroblastoma, breast cancer, glioma, multiple myeloma, or ovarian cancer. In some embodiments, the cancer is multiple myeloma. In some embodiments, the cancer is Ewing sarcoma.

Disclosed herein are methods of treating neurodegenerative diseases comprising administering to a subject in need thereof any one of the compounds disclosed herein, or a pharmaceutically acceptable salt thereof.

In some embodiments, neurodegenerative diseases include, but are not limited to, Alzheimer's disease, multiple sclerosis, Huntington's disease, infectious meningitis, encephalomyelitis, Parkinson's disease, amyotrophic lateral sclerosis, or encephalitis.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention (e.g., compound of formula I or Ia) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s). In some embodiments, the conjoint administration provides a synergistic effect. In some embodiments, the combination index is less than 0.6.

In some embodiments, the additional therapeutic agent is a DNA-damaging agent. In some embodiments, the additional therapeutic agent is a p53 stabilizing agent. In some embodiments, the additional therapeutic agent is selected from RG7388, etoposide, GSK2830371, and doxorubicin.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises”, “comprising”, “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The term “and/or” is used in this disclosure to mean either “and” or “or” unless indicated otherwise.

The term “acyl” is art-recognized and refers to a group represented by the general formula hydrocarbylC(═O)—, preferably alkylC(═O)—.

The term “acylamino” is art-recognized and refers to an amino group substituted with an acyl group and may be represented, for example, by the formula hydrocarbylC(═O)NH—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C1-C6 straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like. Furthermore, as valence permits, “alkyl” also refers to a diradical (e.g., “alkylene”).

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “heteroalkyl”, as used herein, refers to a saturated or unsaturated chain of carbon atoms and at least one heteroatom, wherein no two heteroatoms are adjacent.

Moreover, the term “heteroalkyl” (or “lower heteroalkyl”) as used throughout the specification, examples, and claims is intended to include both “unsubstituted heteroalkyl” and “substituted heteroalkyls”, the latter of which refers to heteroalkyl moieties having substituents replacing a hydrogen on one or more carbons or heteroatoms of the backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the heteroalkyl chain can themselves be substituted, if appropriate. For instance, the substituents of a substituted heteroalkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

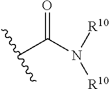

wherein each R¹⁰ independently represents a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

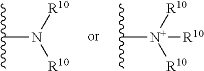

wherein each R¹⁰ independently represents a hydrogen or a hydrocarbyl group, or two R¹⁰ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like. Furthermore, as valence permits, “aryl” also refers to a diradical (e.g., “arylene”).

The term “carbamate” is art-recognized and refers to a group

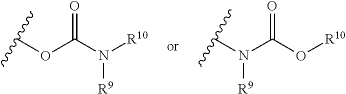

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydroacridine, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. Furthermore, as valence permits, “cycloalkyl” also refers to a diradical (e.g., “cycloalkylene”). The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The terms “carbocyclylalkyl”, as used herein, refers to an alkyl group substituted with a carbocycle group.

The term “carbonate” is art-recognized and refers to a group —OCO2-R10, wherein R10 represents a hydrocarbyl group.

The term “carboxy”, as used herein, refers to a group represented by the formula —CO2H.

The term “ester”, as used herein, refers to a group —C(O)OR10 wherein R10 represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo”, “Hal”, and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like. Furthermore, as valence permits, “heteroaryl” also refers to a diradical (e.g., “heteroarylene”).

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Furthermore, as valence permits, “heterocyclyl” also refers to a diradical (e.g., “heterocyclylene”).

The term “heterocycloalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO3H, or a pharmaceutically acceptable salt thereof.

The term “sulfonamide” is art-recognized and refers to the group represented by the general formulae

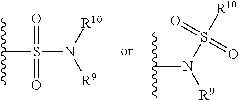

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R10, wherein R10 represents a hydrocarbyl.

The term “sulfonate” is art-recognized and refers to the group SO3H, or a pharmaceutically acceptable salt thereof.

The term “sulfone” is art-recognized and refers to the group —S(O)2-R10, wherein R10 represents a hydrocarbyl.

The term “thioalkyl”, as used herein, refers to an alkyl group substituted with a thiol group.

The term “thioester”, as used herein, refers to a group —C(O)SR10 or —SC(O)R10 wherein R10 represents a hydrocarbyl.

The term “thioether”, as used herein, is equivalent to an ether, wherein the oxygen is replaced with a sulfur.

The term “urea” is art-recognized and may be represented by the general formula

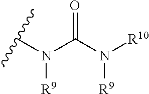

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group.

Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

The present invention includes all pharmaceutically acceptable isotopically-labelled compounds as described herein wherein one or more atoms are replaced by atoms having the same atomic number, but an atomic mass or mass number different from the atomic mass or mass number usually found in nature. In certain embodiments, compounds of the invention are enriched in such isotopically labeled substances (e.g., compounds wherein the distribution of isotopes in the compounds in the composition differ from a natural or typical distribution of isotopes).

Examples of isotopes suitable for inclusion in the compounds of the invention include isotopes of hydrogen, such as ²H and ³H carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ⁵S.

Certain isotopically-labelled compounds as disclosed herein, for example, those incorporating a radioactive isotope, are useful in drug and/or substrate tissue distribution studies. The radioactive isotopes tritium, i.e. ³H, and carbon-14, i.e. ¹⁴C, are useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron-emitting isotopes, such as ¹¹C, ¹⁸F, ¹⁵O and ¹³N, can be useful in Positron Emission Tomography (PET) studies for examining substrate receptor occupancy.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric race mates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. “Diastereomers” are stereoisomers that are not related as mirror images, most commonly because they contain two or more asymmetrically substituted carbon atoms and represent the configuration of substituents around one or more chiral carbon atoms.

Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

Diastereomeric purity by weight is the ratio of the weight of one diastereomer or over the weight of all the diastereomers. When the stereochemistry of a disclosed compound is named or depicted by structure, the named or depicted stereoisomer is at least about 60%, about 70%, about 80%, about 90%, about 99% or about 99.9% by weight relative to the other stereoisomers. When a single enantiomer is named or depicted by structure, the depicted or named enantiomer is at least about 60%, about 70%, about 80%, about 90%, about 99% or about 99.9% by weight optically pure. When a single diastereomer is named or depicted by structure, the depicted or named diastereomer is at least about 60%, about 70%, about 80%, about 90%, about 99% or about 99.9% by weight pure. Percent optical purity is the ratio of the weight of the enantiomer or over the weight of the enantiomer plus the weight of its optical isomer.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

As used herein, the term “pharmaceutically acceptable salt” means any pharmaceutically acceptable salt of the compound of formula (I). For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

In treatment, the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

### EXAMPLES

### Example 1: Preparation of Exemplary Compounds of the Disclosure

### Analytical Methods, Materials, and Instrumentation

All the reagents were commercially obtained in China and used without further purification.

¹H-NMR spectra were recorded in CDCl₃, CD₃OD or DMSO-d6 solutions (reported in ppm) on a Bruker instrument (400 MHz or 500 MHz), using tetramethylsilane (TMS) as the reference standard (0.0 ppm).

Mass spectrometry measurements were carried out using Agilent G6100 series Mass Spectrometer using electrospray ionization source.

Column chromatography was performed on a Biotage system (Manufacturer: Biotage Sweden AB) using silica gel columns.

### Synthesis of Intermediate A-1

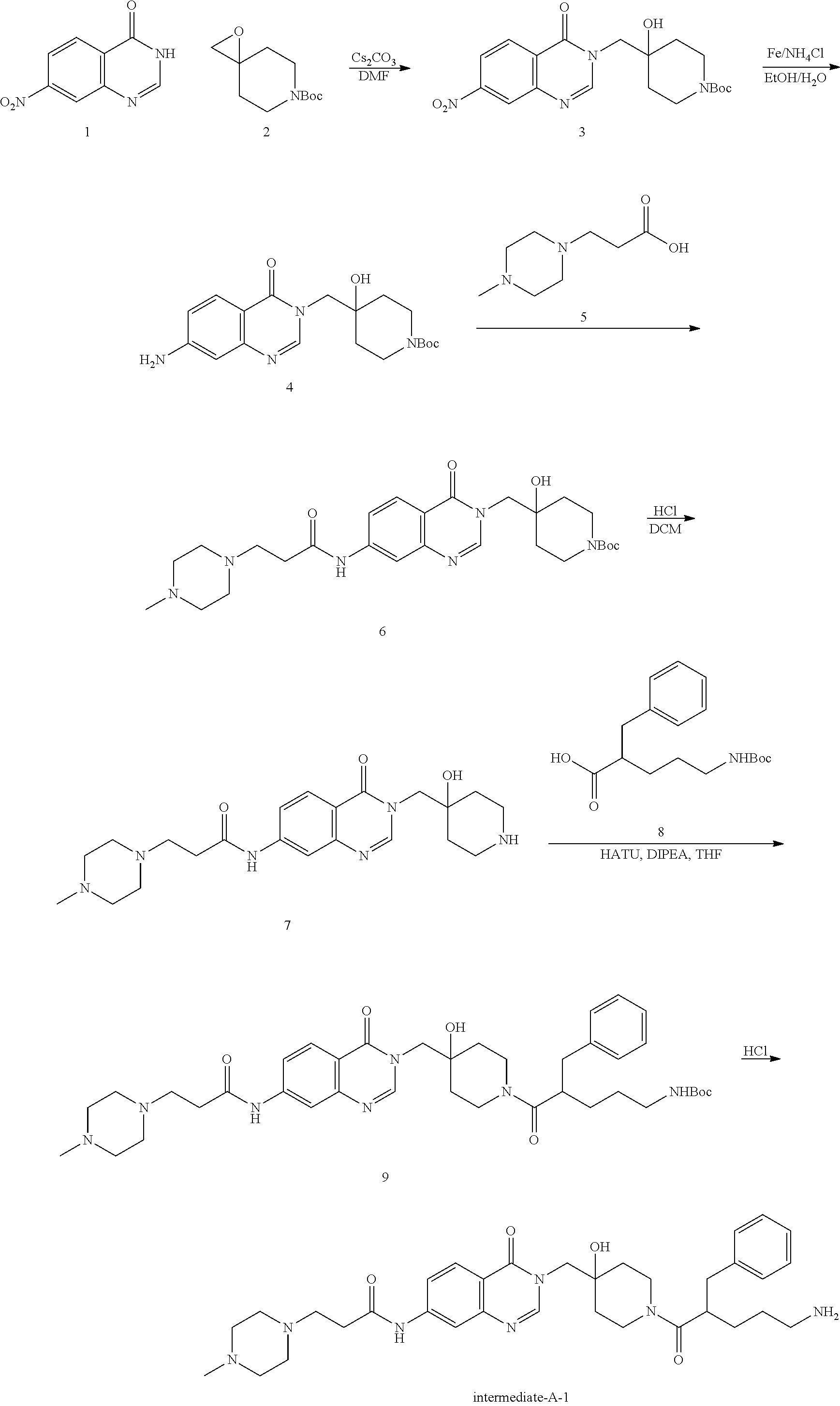

### tert-butyl 4-hydroxy-4-((7-nitro-4-oxoquinazolin-3(4H)-yl)methyl)piperidine-1-carboxylate (Intermediate 3)

A mixture of 7-nitroquinazolin-4(3H)-one (5.0 g, 26.0 mmol) and tert-butyl 1-oxa-6-azaspiro[2.5]octane-6-carboxylate (6.9 g, 32.0 mmol) in DMF (60.0 mL) was stirred at 80° C. for 16 h. After cooled down to rt the mixture was poured into water (200 mL), the resulting solid was collected and further purified by column chromatography on silica-gel (EA\PE=2\1) to afford compound 3 (3.0 g, yield 28%) as yellow solid. LCMS (m/z): 349.1 [M-56+H]⁺.

### tert-butyl 4-((7-amino-4-oxoquinazolin-3(4H)-yl)methyl)-4-hydroxypiperidine-1-carboxylate (Intermediate 4)

A mixture of intermediate 3 (2700 mg, 6.6 mmol), Fe (1848 mg, 6.6 mmol) and NH₄Cl (1848 mg, 6.6 mmol) in EtOH (50.0 mL) and H₂O (6.0 mL) was stirred at 80° C. for 2 h. After cooled down to rt the mixture was diluted with EtOAc (100 mL), washed with saturated NaHCO₃ solution (100 mL), dried over anhydrous Na₂SO₄, filtered and concentrated in vacuum to get compound 4 (2.0 g, yields:83%) as white solid. LCMS (m/z): 375.2 [M+H]⁺.

### tert-butyl 4-hydroxy-4-((7-(3-(4-methylpiperazin-1-yl)propanamido)-4-oxoquinazolin-3(4H)-yl)methyl)piperidine-1-carboxylate (Intermediate 6)

Oxalyl chloride (1142 mg, 9.0 mmol) was added dropwise to the mixture of intermediate 5 (1032 mg, 6.0 mmol) and DMF (0.2 mL) in DCM (30.0 mL) at 0° C., the mixture was stirred at rt for 1 h, the solvent was removed under vacuum, the residue was dissolved in DCM (20 mL) and added dropwise to the solution of intermediate 4 (748 mg, 2.0 mmol), Et₃N (1200 mg, 12.0 mmol) and DMAP (24 mg, 0.2 mmol) in DCM (30.0 mL) at 0° C., the mixture was stirred at 0° C. for 2 h, diluted with saturated NaHCO₃ solution (100 mL) and extracted with DCM (100 mL×2), the combined organic was dried over anhydrous Na₂SO₄, concentrated and purified by column chromatography on silica-gel (MeOH/DCM=1/20) to afford intermediate 6 (1000 mg, yields: 65%) as yellow solid. LCMS (m/z): 529.3 [M+H]⁺.

### N-(3-((4-hydroxypiperidin-4-yl)methyl)-4-oxo-3,4-dihydroquinazolin-7-yl)-3-(4-methylpiperazin-1-yl)propanamide (Intermediate 7)

A mixture of intermediate 6 (1000 mg, 1.9 mmol), HCl-dioxane (4 M, 9.5 mL) in DCM (9.0 mL) was stirred at rt for 2 h. The mixture was concentrated under vacuum to leave crude intermediate 7 (900 mg, yield 100%) as white solid. LCMS (m/z): 429.2 [M+H]⁺.

### tert-butyl (4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)carbamate (Intermediate 9)

A mixture of intermediate 8 (645 mg, 2.1 mmol), HATU (878 mg, 2.3 mmol) and DIPEA (1.8 mL, 10.5 mmol) in DCM (20.0 mL) was stirred at rt for 1 h, the mixture was then added dropwise to the mixture of intermediate 7 (900 mg, 2.1 mmol) and DIPEA (1.8 mL, 10.5 mmol) in DCM (20.0 mL), the resulting mixture was stirred at rt for 2 h, diluted with water (100 mL) and extracted with DCM (100 mL×2), the combined organic was dried over anhydrous Na₂SO₄, concentrated and purified by column chromatography on silica-gel (MeOH/DCM=1/10) to afford intermediate 9 (1.4 g, yield 93%) as yellow solid. LCMS (m/z): 718.5 [M+H]⁺

### N-(3-((1-(5-amino-2-benzylpentanoyl)-4-hydroxypiperidin-4-yl)methyl)-4-oxo-3,4-dihydroquinazolin-7-yl)-3-(4-methylpiperazin-1-yl)propanamide (Intermediate A-1)

To a mixture of intermediate 9 (1400 mg, 1.9 mmol) in DCM (10.0 mL) was added HCl solution in 1,4-dioxane (4 M, 5.0 mL), the mixture was stirred at rt for 2 h and concentrated in vacuum to afford Intermediate A-1 (1.4 g, yield 100%) as white solid. LCMS (m/z): 618.3 [M+H]⁺

### Synthesis of Intermediate C

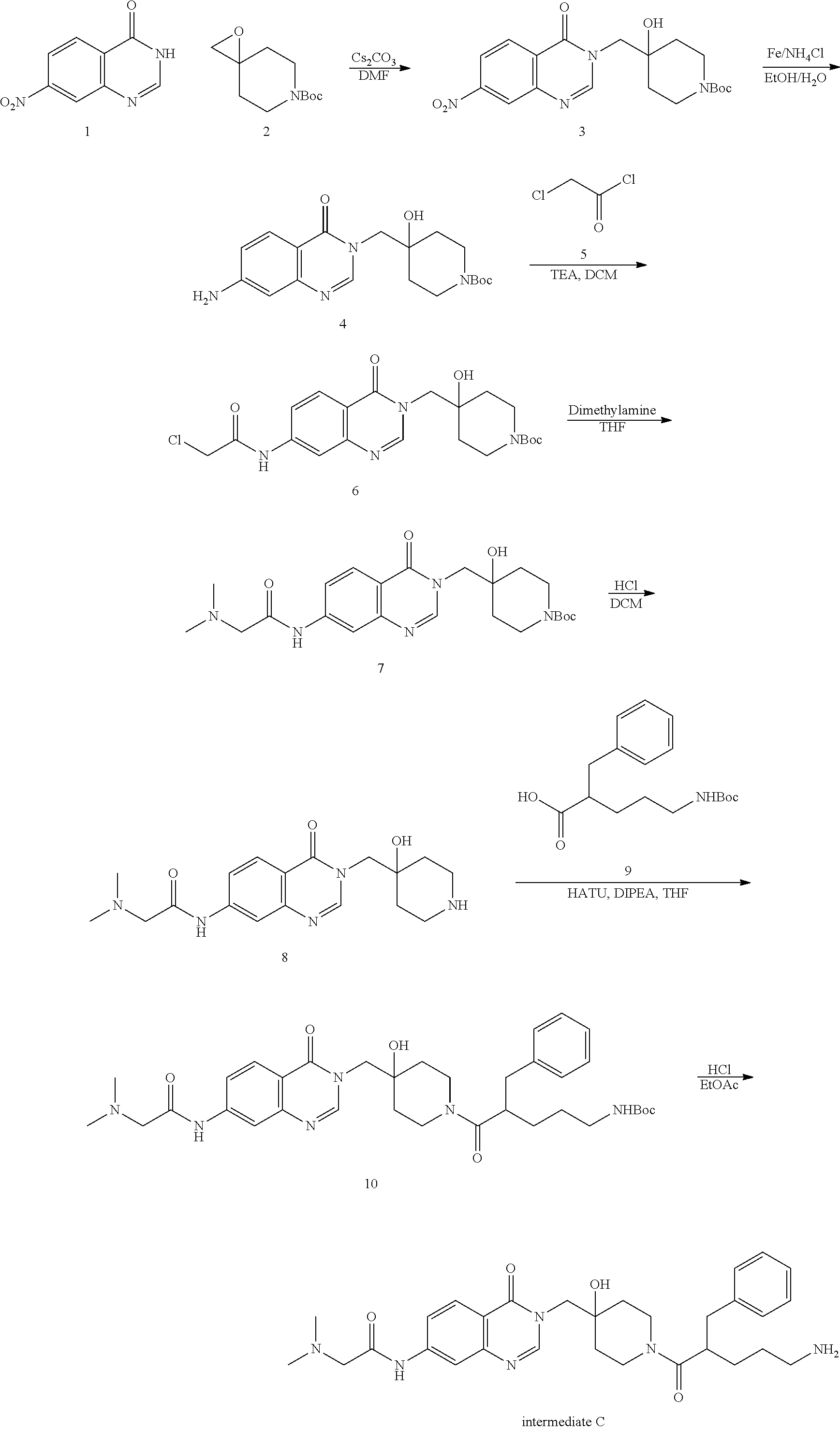

### tert-butyl 4-hydroxy-4-((7-nitro-4-oxoquinazolin-3(4H)-yl)methyl)piperidine-1-carboxylate (Intermediate 3)

A mixture of 7-nitroquinazolin-4(3H)-one (5.0107 g, 26.21 mmol, 1.0 eq), tert-butyl 1-oxa-6-azaspiro[2.5]octane-6-carboxylate (6.7786 g, 31.78 mmol, 1.2 eq) and Cs₂CO₃ (12.7185 g, 39.04 mmol, 1.5 eq) in DMF (60 mL) was stirred at 80° C. for 16 h. The resulting mixture was poured into iced water (200 mL). Ethyl acetate (500 mL) was added into the resulting mixture. Layers separated and the aqueous phase was extracted with ethyl acetate (150 mL). The organic phase was washed with saturated NaCl solution and dried over Na₂SO₄. The filtrate was concentrated by rotary evaporation (40° C.) to afford the target intermediate 3 (9.88 g, 24.455 mmol, 93.31% yield) as brown solid. LCMS (m/z): 349.3 [M-55]⁺.

### tert-butyl 4-((7-amino-4-oxoquinazolin-3(4H)-yl)methyl)-4-hydroxypiperidine-1-carboxylate (Intermediate 4)

A mixture of intermediate 3 (9.88 g, 24.445 mmol, 1.0 eq), Fe (6.8292 g, 122.28 mmol, 5.0 eq) and NH₄Cl (6.5353 g, 122.18 mmol, 5.0 eq) in ethanol/H₂O (75 mL/15 mL) was stirred at 80° C. for 2 h. Ethyl acetate (100 mL) was added while stirring and the resulting mixture was filtered under vacuum. The filtrate was extracted with ethyl acetate (50 mL×3) and the combined organic layer was washed with saturated NaCl solution (100 mL). The organic was concentrated and purified by flash column chromatography (DCM/MeOH=10/1) to afford the target intermediate 4 (6.48 g, 17.326 mmol, 70.88% yield) as dark brown solid. LCMS (m/z): 375.3 [M+H]⁺.

### tert-butyl 4-((7-(2-chloroacetamido)-4-oxoquinazolin-3(4H)-yl)methyl)-4-hydroxypiperidine-1-carboxylate (Intermediate 6)

A mixture of intermediate 5 (0.112 g, 1.0 mmol, 2.0 eq) in anhydrate DCM (10 mL) was added drop by drop into a solution of 4 (0.1871 g, 0.5 mmol, 1.0 eq) and Et₃N (0.32 mL, 3.0 eq) in anhydrate DCM (10 mL) while stirring in an ice-water bath. The ice-water bath was then removed and the resulting mixture was stirred at room temperature for another 4 hours. Water (10 mL) and DCM (50 mL) were added into the reaction mixture and layers separated. The aqueous was extracted with DCM (50 mL×3). The combined organic phase was washed with saturated NaCl solution (10 mL), dried over Na₂SO₄ and filtered. The filtrate was concentrated by rotary evaporation (30° C.) to afford the target intermediate 6 (0.272 g, 0.604 mmol, 60.44% yield) as dark grey solid. LCMS (m/z): 451.1 [M+H]⁺.

### tert-butyl 4-((7-(2-(dimethylamino)acetamido)-4-oxoquinazolin-3(4H)-yl)methyl)-4-hydroxypiperidine-1-carboxylate (Intermediate 7)

In a microwave tube, a mixture of dimethylamine (2 M, 3 mL, 6 mmol, 4.0 eq) and intermediate 6 (0.675 g, 1.5 mmol, 1.0 eq) in THF (8 mL) was stirred at 25° C. for 16 hours. The organic solvent was removed under vacuum. Ethyl acetate (200 mL) was added into the reaction mixture. The mixture was then washed with water (10 mL) and layers separated. The aqueous phase was extracted with ethyl acetate (100 mL×3). The combined organic phase was washed with saturated NaCl solution (100 mL), dried over Na₂SO₄ and filtered. The filtrate was concentrated using rotary evaporation (40° C.) to afford intermediate 7 (0.645 g, 1.404 mmol, 93.62% yield) as dark brown solid. LCMS (m/z): 460.3 [M+H]⁺.

### 2-(dimethylamino)-N-(3-((4-hydroxypiperidin-4-yl)methyl)-4-oxo-3,4-dihydroquinazolin-7-yl)acetamide (Intermediate 8)

A mixture of intermediate 7 (0.1191 g, 0.259 mmol, 1.0 eq) and HCl solution in 1,4-dioxane (4 M, 2 mL, 20.0 eq) in DCM (8 mL) was stirred at room temperature for 2 hours. The reaction mixture was concentrated using rotary evaporation (50° C.) to afford intermediate 8 (0.093 g, 0.259 mmol, 99.86% yield) as white solid. LC-MS (m/z): 360.2 [M+H]⁺.

### tert-butyl 4-benzyl-5-(4-((7-(2-(dimethylamino)acetamido)-4-oxoquinazolin-3(4H)-yl)methyl)-4-hydroxypiperidin-1-yl)-5-oxopentylcarbamate (Intermediate 10)

A mixture of intermediate 8 (0.6636 g, 1.847 mmol, 1.2 eq) and DIPEA (0.4200 g, 3.250 mmol, 3.0 eq) in THF (10 mL) was stirred at room temperature while a mixture of intermediate 9 (0.3312 g, 1.078 mmol, 1.0 eq), HATU (0.4532 g, 1.192 mmol, 1.2 eq) and DIPEA (0.2633 g, 2.037 mmol, 2.0 eq) in THF (15 mL) was stirred at room temperature. After an hour, the two mixtures were mixed together and the resulting mixture was stirred at room temperature for another 2 hours. Water (10 mL) and DCM (100 mL) were added into the reaction mixture and layers separated. The aqueous phase was extracted with DCM (100 mL×3). The combined organic phase was washed with saturated NaCl solution (100 mL), dried over Na₂SO₄ and filtered. The filtrate was concentrated and purified by flash column chromatography (EA/MeOH=9/1) to afford intermediate 10 (0.453 g, 0.699 mmol, 64.85% yield) as yellow solid. LC-MS (m/z): 649.4 [M+H]⁺.

### N-(3-((1-(5-amino-2-benzylpentanoyl)-4-hydroxypiperidin-4-yl)methyl)-4-oxo-3,4-dihydroquinazolin-7-yl)-2-(dimethylamino)acetamide (Intermediate C)

A mixture of intermediate 10 (0.453 g, 0.7 mmol, 1.0 eq) and HCl/1,4-dioxane (4 M, 3.5 mL, 20.0 eq) in EtOAc (10 mL) was stirred at room temperature for 5 hours. The solvent was then removed under vacuum to afford the target compound Intermediate C (0.3832 g, 0.699 mmol, 100% yield) as white solid. LC-MS (m/z): 649.4 [M+H]⁺.

### Synthesis of Compounds 4 and 5

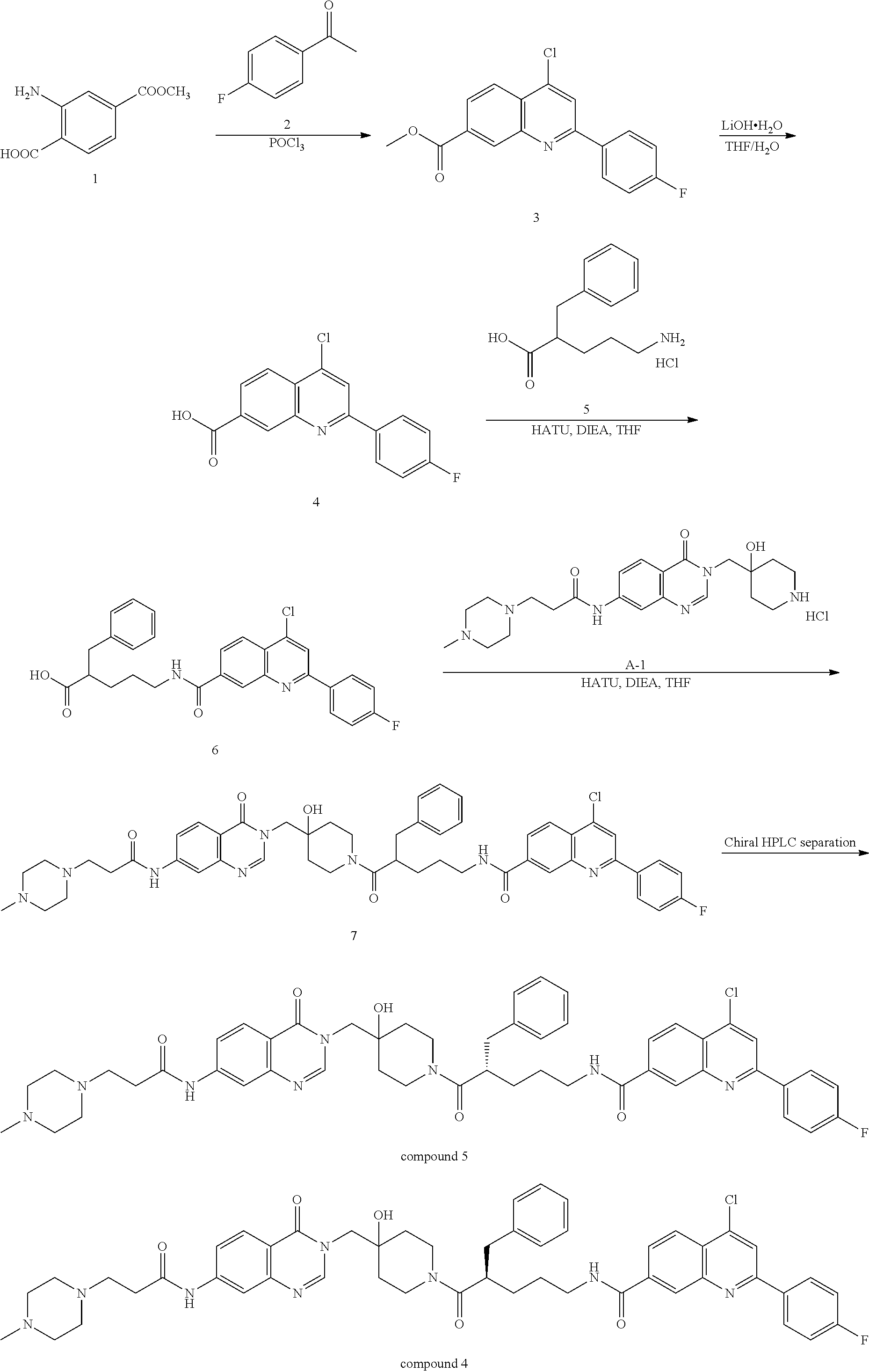

### methyl 4-chloro-2-(4-fluorophenyl)quinoline-7-carboxylate (Intermediate 3)

A mixture of 2-amino-4-(methoxycarbonyl)benzoic acid (585 mg, 5.0 mmol) and 1-(4-fluorophenyl)ethanone (1036 mg, 7.5 mmol) in POCl₃ (10 mL) was stirred at 100° C. for 3 hours, after cooled down to rt, the mixture was concentrated in vacuum, the residue was taken into ice water (20 mL), adjusted to pH 8-9 with solid NaHCO₃, and extracted with DCM (3×50 mL). The combined organics was dried over anhydrous Na₂SO₄, concentrated and purified by flash chromatograph on silica gel (DCM/MeOH=40/1) to give intermediate 3 (174 mg, Yield 11%). LCMS: (m/z) 316.0 [M+H]⁺

### 4-chloro-2-(4-fluorophenyl)quinoline-7-carboxylic acid (Intermediate 4)

To a solution of methyl 4-chloro-2-(4-fluorophenyl)quinoline-7-carboxylate (255 mg, 0.808 mmol) in THF (20 mL) was added a solution of LiOH·H₂O (68 mg, 1.616 mmol) in H₂O (5 mL). The mixture was stirred at rt for 16 hours and the solvent was removed in vacuum. The residue was taken up in water and acidified with 1 M HCl to pH 4-5, the resulting mixture was extracted with ethyl acetate (3×50 mL), dried over anhydrous Na₂SO₄, filtered and concentrated in vacuum to leave the crude product (233 mg, Yield 95.8%) as a thick gum. LCMS: (m/z) 330.2 [M+Na]⁺

### 2-benzyl-5-(4-chloro-2-(4-fluorophenyl) quinoline-7-carboxamido)pentanoic acid (Intermediate 6)

The mixture of 4-chloro-2-(4-fluorophenyl)quinoline-7-carboxylic acid (203 mg, 0.67 mmol), HATU (255 mg, 0.67 mmol) and DIEA (173 mg, 1.34 mmol) in THF (10 mL) was stirred at rt for 30 minutes, and then added into a mixture of DIEA (134 mg, 1.34 mmol) and 5-amino-2-benzylpentanoic acid hydrochloride (163 mg, 0.67 mmol) in THF (10 mL). The mixture was stirred at rt for 16 hours concentrated and purified by flash column chromatograph (DCM/CH₃OH=20/1) to give intermediate 6 (265 mg, Yield 70%) as white solid. LCMS: (m/z) 491.2 [M+H]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(4-fluorophenyl)quinoline-7-carboxamide (Intermediate 7)

A mixture of N-(3-((4-hydroxypiperidin-4-yl)methyl)-4-oxo-3,4-dihydroquinazolin-7-yl)-3-(4-methylpiperazin-1-yl)propanamide hydrochloride (240 mg, 0.49 mmol), 2-benzyl-5-(4-chloro-2-(4-fluorophenyl)quinoline-7-carboxamido)pentanoic acid (210 mg, 0.49 mmol), HATU (205 mg, 0.539 mmol) and DIEA (190 mg, 1.47 mmol) in THF (10 mL) was stirred at rt for 16 hours. The mixture was concentrated in vacuum, the residue was purified by flash chromatograph (DCM/MeOH=10/1) to give crude product which was further purified by prep-HPLC (C18 column, CH₃CN/H₂O, containing 0.05% NH₄HCO₃) to give intermediate 7 (115 mg, Yield 26%) as a white solid. LCMS: (m/z) 901.8 [M+H]⁺

### (S)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(4-fluorophenyl)quinoline-7-carboxamide (Compound 5)

### (R)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(4-fluorophenyl)quinoline-7-carboxamide (Compound 4)

N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(4-fluorophenyl)quinoline-7-carboxamide (92 mg, 0.102 mmol) was purified by Chiral-HPLC (Instrument: Gilson-281, Column: SA 20*250, 10 um, Mobile Phase: MEOH (0.2% Methanol Ammonia):ACN (0.2% Methanol Ammonia)=70:30, FlowRate: 40 ml/min, Run time per injection: 22 min, Injection: 1.5 ml, Sample solution: 100 mg in 45 mL MeOH) to give compound 5 (29.5 mg) and compound 4 (9.0 mg) as solid.

### Compound 5:

¹H NMR (500 MHz, DMSO-d₆) δ (ppm) 10.57 (s, 1H), 9.15 (s, 1H), 8.94-8.86 (m, 1H), 8.65-8.61 (m, 1H), 8.50-8.35 (m, 3H), 8.28-8.23 (m, 1H), 8.17-7.97 (m, 4H), 7.65-7.57 (m, 1H), 7.44-7.35 (m, 2H), 7.27-7.07 (m, 4H), 4.88 (s, 1H), 4.17-3.99 (m, 1H), 3.91-3.57 (m, 3H), 3.31-3.10 (m, 4H), 2.89-2.52 (m, 9H), 2.47-2.20 (m, 6H), 2.13 (s, 3H), 1.72-1.03 (m, 8H), LCMS: (m/z) 901.4 [M+H]⁺,

### Compound 4:

¹H NMR (400 MHz, DMSO-d₆) δ (ppm) 8.99-8.88 (m, 1H), 8.66-8.63 (m, 1H), 8.54-8.47 (m, 1H), 8.45-8.36 (m, 2H), 8.30-8.21 (m, 1H), 8.20-7.98 (m, 4H), 7.69-7.58 (m, 1H), 7.46-7.35 (m, 2H), 7.27-7.07 (m, 4H), 4.18-3.97 (m, 1H), 3.94-3.38 (m, 6H), 3.23-3.29 (m, 4H), 2.91-2.54 (m, 6H), 2.45-2.18 (m, 6H), 2.13 (s, 3H), 1.71-1.00 (m, 8H), LCMS: (m/z) 901.4 [M+H]⁺,

### Synthesis of Compound 6

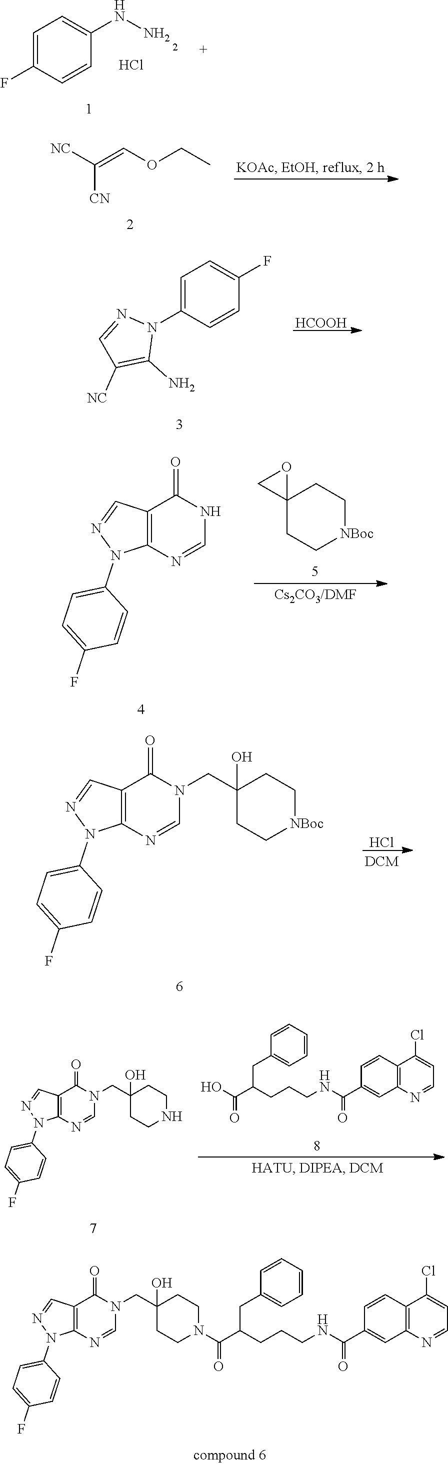

### 5-amino-1-(4-fluorophenyl)-1H-pyrazole-4-carbonitrile (Intermediate 3)

A mixture of (4-fluorophenyl)hydrazine (1.6 g, 10.0 mmol) and KOAc (0.98 g, 10.0 mmol) in EtOH (80.0 mL) was stirred at 80° C. for 20 min, and then 2-(ethoxymethylene)malononitrile (1.2 g, 10.0 mmol) was added, the mixture was stirred at 80° C. for 2 hour, concentrated and purified by column chromatography on silica-gel (EA/PE=1/3) to afford intermediate 3 (1.5 g, yield: 75%) as yellow solid. LCMS (m/z): 203.1 [M+H]⁺.

### 1-(4-fluorophenyl)-1,5-dihydro-4H-pyrazolo[3,4-d]pyrimidin-4-one (Intermediate 4)

A mixture of intermediate 3 (808 mg, 4.0 mmol) and HCOOH (8.0 mL) was stirred at 100° C. for 16 hours. After cooled down to rt, the mixture was diluted with ice-cold water (100 mL), filtered and dried in the air to give intermediate 4 (600 mg, yield: 65%) as white solid. LCMS (m/z): 231.1 [M+H]⁺.

### tert-butyl 4-((1-(4-fluorophenyl)-4-oxo-1,4-dihydro-5H-pyrazolo[3,4-d]pyrimidin-5-yl)methyl)-4-hydroxypiperidine-1-carboxylate (Intermediate 6)

A mixture of intermediate 4 (400 mg, 1.45 mmol), tert-butyl 1-oxa-6-azaspiro[2.5]octane-6-carboxylate (370 mg, 1.73 mmol) and Cs₂CO₃ (709 mg, 2.1 mmol) in DMF (15.0 mL) was stirred at 80° C. for 16 h. After cooled down to rt, the mixture was diluted with water (100 mL) and extracted with EtOAc (100 mL*2), the combined organic was dried over anhydrous Na₂SO₄, filtered, concentrated and purified by by column chromatography on silica-gel (EA/PE=1/2) to obtained intermediate 6 (500 mg, yield: 77.7%) as white solid. LCMS (m/z): 344.1 [M-100+H]⁺.

### 1-(4-fluorophenyl)-5-((4-hydroxypiperidin-4-yl)methyl)-1,5-dihydro-4H-pyrazolo[3,4-d]pyrimidin-4-one (Intermediate 7)

A mixture of intermediate 6 (60 mg, 0.13 mmol) and HCl solution in 1,4-dioxane (4 M, 0.6 mL) in DCM (6.0 mL) was stirred at rt for 2 h, the mixture was concentrated to leave the crude intermediate 7 (60 mg, 100%) as white solid. LCMS (m/z): 344.1 [M+H]⁺.

### N-(4-benzyl-5-(4-((1-(4-fluorophenyl)-4-oxo-1,4-dihydro-5H-pyrazolo[3,4-d]pyrimidin-5-yl)methyl)-4-hydroxypiperidin-1-yl)-5-oxopentyl)-4-chloroquinoline-7-carboxamide (Compound 6)

A mixture of 2-benzyl-5-(4-chloroquinoline-7-carboxamido) pentanoic acid (intermediate 8) (39.6 mg, 0.1 mmol), compound 7 (34.3 mg, 0.1 mmol), HATU (41.8 mg, 0.11 mmol) and DIPEA (0.1 mL, 0.5 mmol) in DCM (6.0 mL) was stirred at rt for 1 h, the mixture was diluted with DCM (30 mL), washed with water (20.0 mL), dried over anhydrous Na₂SO₄, filtered, concentrated and purified by prep-HPLC (C18 column, CH₃CN/H₂O, containing 0.05% NH₄HCO₃) to obtain compound 6 (40 mg, yield 55%) as white solid. ¹H NMR (DMSO-d₆, 400 MHz): δ (ppm) 8.94-8.88 (m, 2H), 8.61-8.59 (m, 1H), 8.39-8.35 (m, 1H), 8.29-8.25 (m, 2H), 8.20-8.15 (m, 1H), 8.09-8.04 (m, 2H), 7.86 (d, J=4.8 Hz, 1H), 7.45-7.40 (m, 2H), 7.29-7.12 (m, 5H), 4.84 (s, 1H), 4.17-3.83 (m, 2H), 3.72-3.59 (m, 2H), 3.32-3.10 (m, 3H), 2.86-2.51 (m, 3H), 1.70-1.05 (m, 9H). LCMS (m/z): 722.2 [M+H]⁺

### Synthesis of Compound 7

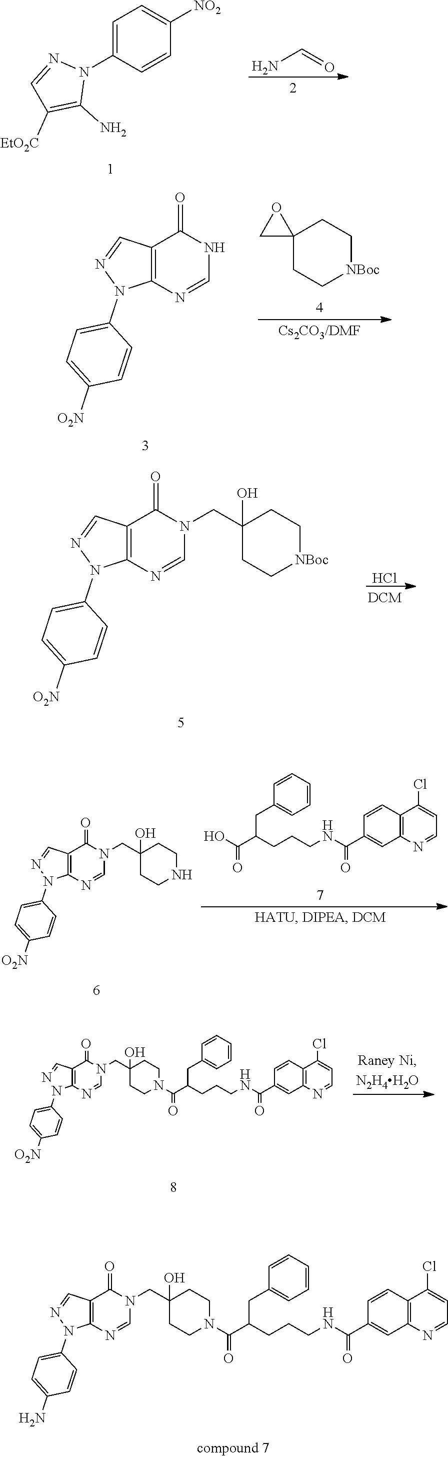

### 1-(4-nitrophenyl)-1,5-dihydro-4H-pyrazolo[3,4-d]pyrimidin-4-one (Intermediate 3)

The mixture of ethyl 5-amino-1-(4-nitrophenyl)-1H-pyrazole-4-carboxylate (intermediate 1) (500 mg, 1.8 mmol) in formamide (10.0 mL) was stirred at 150° C. for 8 h, after cooled down to rt, the mixture was poured into ice-cold water (100 mL), the resulting solid was filtered and dried in the air to get intermediate 3 (400 mg, yields: 86%) as brown solid. LCMS (m/z): 258.1 [M+H]⁺.

### tert-butyl 4-hydroxy-4-((1-(4-nitrophenyl)-4-oxo-1,4-dihydro-5H-pyrazolo[3,4-d]pyrimidin-5-yl)methyl)piperidine-1-carboxylate (Intermediate 5)

A mixture of intermediate 3 (400 mg, 1.5 mmol), tert-butyl 1-oxa-6-azaspiro[2.5]octane-6-carboxylate (490 mg, 2.3 mmol) and Cs₂CO₃ (758 mg, 2.3 mmol) in DMF (10.0 mL) was stirred at 80° C. for 16 h. After cooled down to rt the mixture was diluted with water (100 mL) and extracted with EtOAc (100 mL*2), the combined organic was dried over anhydrous Na₂SO₄, filtered, concentrated and purified by column chromatography on silica-gel (EA/PE=1/2) to obtained compound intermediate 5 (130 mg, yields: 18%) as white solid. LCMS (m/z): 371.1 [M-100+H]⁺.

### 5-((4-hydroxypiperidin-4-yl)methyl)-1-(4-nitrophenyl)-1,5-dihydro-4H-pyrazolo[3,4-d]pyrimidin-4-one (Intermediate 6)

A mixture of intermediate 5 (120 mg, 0.25 mmol) and) and HCl solution in 1,4-dioxane (4 M, 1.5 mL) in DCM (6.0 mL) was stirred at rt for 2 h, the mixture was concentrated to leave the crude product, which was further purified by prep-HPLC to afford the pure intermediate 6 (40 mg, yields: 43%) as yellow solid. LCMS (m/z): 371.2 [M+H]⁺.

### N-(4-benzyl-5-(4-hydroxy-4-((1-(4-nitrophenyl)-4-oxo-1,4-dihydro-5H-pyrazolo[3,4-d]pyrimidin-5-yl)methyl)piperidin-1-yl)-5-oxopentyl)-4-chloroquinoline-7-carboxamide (Intermediate 8)

A mixture of intermediate 7 (39.6 mg, 0.1 mmol), intermediate 6 (37 mg, 0.1 mmol), HATU (41.8 mg, 0.11 mmol) and DIPEA (0.1 mL, 0.5 mmol) in DCM (6.0 mL) was stirred at rt for 2 h, the suspension was filtered, the cake was dried in the air to get intermediate 8 (60 mg, yields: 80%) as white solid. LCMS (m/z): 749.4 [M+H]⁺.

### N-(5-(4-((1-(4-aminophenyl)-4-oxo-1,4-dihydro-5H-pyrazolo[3,4-d]pyrimidin-5-yl)methyl)-4-hydroxypiperidin-1-yl)-4-benzyl-5-oxopentyl)-4-chloroquinoline-7-carboxamide (Intermediate 7)

A mixture of intermediate 8 (60 mg, 0.08 mmol), Raney Ni (30 mg) and N₂H₄·H₂O (0.9 mL) in EtOH (3.0 mL) and THF (3.0 mL) was stirred at rt for 3 h, the mixture was filtered, the filtrate was concentrated and purified by prep-HPLC (C18 column, CH₃CN/H₂O, containing 0.05% NH₄HCO₃) to give intermediate 7 (21 mg, yield 36%) as a white solid. ¹H NMR (DMSO-d₆, 400 MHz): δ (ppm) 8.93-8.88 (m, 2H), 8.61-8.59 (m, 1H), 8.28-8.16 (m, 4H), 7.86 (d, J=4.4 Hz, 1H), 7.53-7.51 (m, 2H), 7.26-7.12 (m, 5H), 6.68 (d, J=8.0 Hz, 2H), 5.39 (s, 2H), 4.81 (s, 1H), 4.16-3.61 (m, 4H), 3.15-3.13 (m, 3H), 2.86-2.62 (m, 4H), 1.65-1.09 (m, 8H). LCMS (m/z): 719.2 [M+H]⁺;

### Synthesis of Compound 8

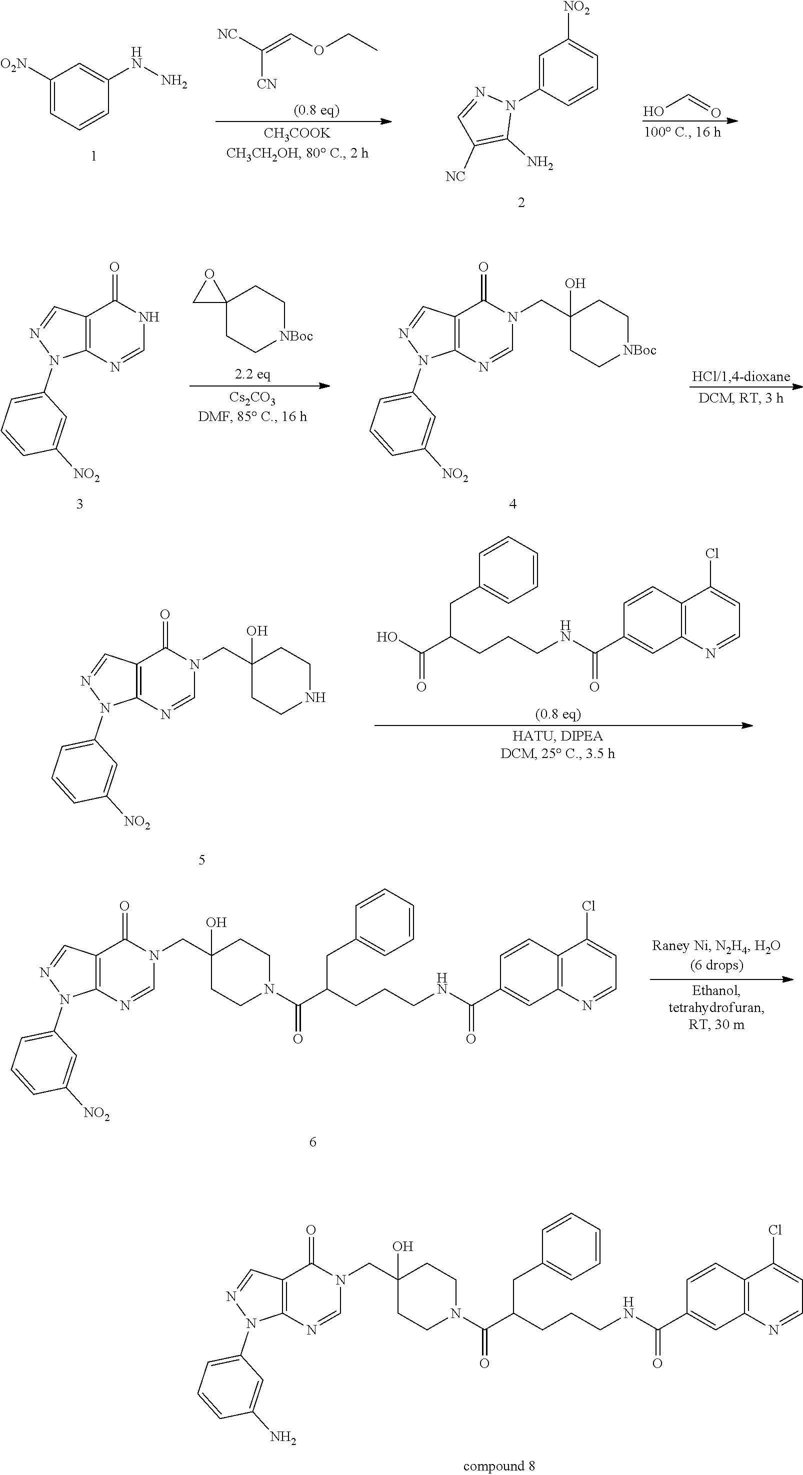

### 5-amino-1-(3-nitrophenyl)-1H-pyrazole-4-carbonitrile (Intermediate 2)

A mixture of (3-nitrophenyl)hydrazine (1002.8 mg, 5.289 mmol, 1.0 eq) and KOAc (517.3 mg, 5.271 mmol, 0.8 eq) in CH₃CH₂OH (50 mL) was stirred in a round bottom flask at 80° C. for 30 minutes. Then 5-amino-1-(3-nitrophenyl)-1H-pyrazole-4-carbonitrile (537.1 mg, 4.398 mmol, 0.8 eq) was added into the reaction mixture, the resulting mixture was stirred at 80° C. for another 2 hours. After filtration, the filtrate was concentrated under vacuum to dryness and purified by flash column chromatography (EA/PE=2/8) to afford intermediate 2 as brown solid (0.236 g, 19.47% yield). LCMS: (m/z) 230.7 [M+H]⁺

### 1-(3-nitrophenyl)-1H-pyrazolo[3,4-d]pyrimidin-4(5H)-one (Intermediate 3)

In a round bottom flask, 5-amino-1-(3-nitrophenyl)-1H-pyrazole-4-carbonitrile (236 mg, 1.030 mmol, 1.0 q) was dissolved in formic acid (15 mL). The resulting mixture was stirred at 100° C. for 16 hours. The reaction mixture was poured into iced water (50 mL) while stirring, the resulting solid was collected and dried under vacuum to afford intermediate 3 as white solid (93 mg, 35.15% yield). LCMS: (m/z) 257.7 [M+H]⁺

### tert-butyl 4-hydroxy-4-((1-(3-nitrophenyl)-4-oxo-1H-pyrazolo[3,4-d]pyrimidin-5(4H)-yl)methyl)piperidine-1-carboxylate (Intermediate 4)

A mixture of 1-(3-nitrophenyl)-1H-pyrazolo[3,4-d]pyrimidin-4(5H)-one (169 mg, 0.657 mmol, 1.0 eq), tert-butyl 1-oxa-6-azaspiro[2.5]octane-6-carboxylate (308.6 mg, 1.446 mmol, 2.2 eq) and Cs₂CO₃ (535.2 mg, 1.643 mmol, 2.5 eq) was stirred at 85° C. for 16 hours. Ethyl acetate (100 mL) was added into the reaction mixture and the resulting mixture was washed with water (10 mL×4). The aqueous was extracted with ethyl acetate (50 mL×6). The combined organic phase was washed with saturated NaCl solution (10 mL) and dried over Na₂SO₄. After filtration, the filtrate was concentrated by rotary evaporation (40° C.). The crude residue was purified by flash column chromatography (EA/PE=1/2) to afford intermediate 4 as white solid (231 mg, 0.491 mmol, 74.78% yield). LC-MS: (m/z) 371.1 [M-100+H]⁺

### 5-((4-hydroxypiperidin-4-yl)methyl)-1-(3-nitrophenyl)-1H-pyrazolo[3,4-d]pyrimidin-4(5H)-one (Intermediate 5)

To a solution of tert-butyl 4-hydroxy-4-((1-(3-nitrophenyl)-4-oxo-1H-pyrazolo[3,4-d]pyrimidin-5(4H)-yl)methyl)piperidine-1-carboxylate (231 mg, 0.491 mmol, 1.0 eq) in dichloromethane (5 mL) was added HCl solution in 1,4-dioxane (4 M, 15 mL, 20.0 eq), the mixture was stirred at room temperature for 3 hours, and then concentrated un vacuum to afford intermediate 5 as white solid (181.7 mg, 0.491 mmol, 100% yield). LC-MS: (m/z) 371.1 [M+H]⁺

### N-(4-benzyl-5-(4-hydroxy-4-((1-(3-nitrophenyl)-4-oxo-1H-pyrazolo[3,4-d]pyrimidin-5(4H)-yl)methyl)piperidin-1-yl)-5-oxopentyl)-4-chloroquinoline-7-carboxamide (Intermediate 6)

A mixture of 2-benzyl-5-(4-chloroquinoline-7-carboxamido)pentanoic acid (57.0 mg, 0.144 mmol, 0.8 eq), HATU (65.5 mg, 0.172 mmol, 1.0 eq) and DIPEA (93.0 mg, 0.720 mmol, 5.0 eq) in dichloromethane (2 mL) was stirred at 25° C. for 30 minutes. Then 5-((4-hydroxypiperidin-4-yl)methyl)-1-(3-nitrophenyl)-1H-pyrazolo[3,4-d]pyrimidin-4(5H)-one (67.8 mg, 0.183 mmol, 1.0 eq) was added into the reaction mixture and the resulting mixture was stirred at 25° C. for another 3.5 hours. Dichloromethane (50 mL) and water (10 mL) were added into the reaction mixture and layers separated. The aqueous phase was extracted with dichloromethane (50 mL×3). The combined organic phase was washed with saturated NaCl solution (10 mL) and dried over Na₂SO₄. After filtration, the filtrate was concentrated by rotary evaporation (40° C.). The crude residue was purified by flash column chromatography (DCM/MeOH=1/5) to afford intermediate 6 as white solid (115 mg, 0.153 mmol, 83.61% yield). LCMS (m/z): 749.2 [M+H]⁺

### N-(5-(4-((1-(3-aminophenyl)-4-oxo-1H-pyrazolo[3,4-d]pyrimidin-5(4H)-yl)methyl)-4-hydroxypiperidin-1-yl)-4-benzyl-5-oxopentyl)-4-chloroquinoline-7-carboxamide (Compound 8)

A mixture of N-(4-benzyl-5-(4-hydroxy-4-((1-(3-nitrophenyl)-4-oxo-1H-pyrazolo[3,4-d]pyrimidin-5(4H)-yl)methyl)piperidin-1-yl)-5-oxopentyl)-4-chloroquinoline-7-carboxamide (97 mg, 0.1296 mmol, 1.0 eq), Raney Ni (100 mg) and N₂H₄H₂O (6 drops) in ethanol (2 mL) and tetrahydrofuran (2 mL) was stirred at room temperature for 30 minutes. The reaction mixture was then concentrated by rotary evaporation (40° C.) and further purified by prep-HPLC (C18 column, CH₃CN/H₂O, containing 0.05% NH₄HCO₃) to afford the target compound 8 (10.6 mg, 0.015 mmol, 11.39% yield) as white solid. ¹H NMR (DMSO-d₆, 400 MHz): δ (ppm) 8.94-8.89 (m, 2H), 8.62-8.58 (m, 1H), 8.32-8.15 (m, 4H), 7.86 (d, J=4.4 Hz, 1H), 7.28-7.12 (m, 8H), 6.57 (br, 1H), 5.44 (s, 2H), 4.83 (s, 1H), 4.16-3.58 (m, 4H), 3.31-3.09 (m, 3H), 2.86-2.62 (m, 4H), 1.67-1.07 (m, 8H). LCMS (m/z): 719.3 [M+H]⁺

### Synthesis of Compound 9

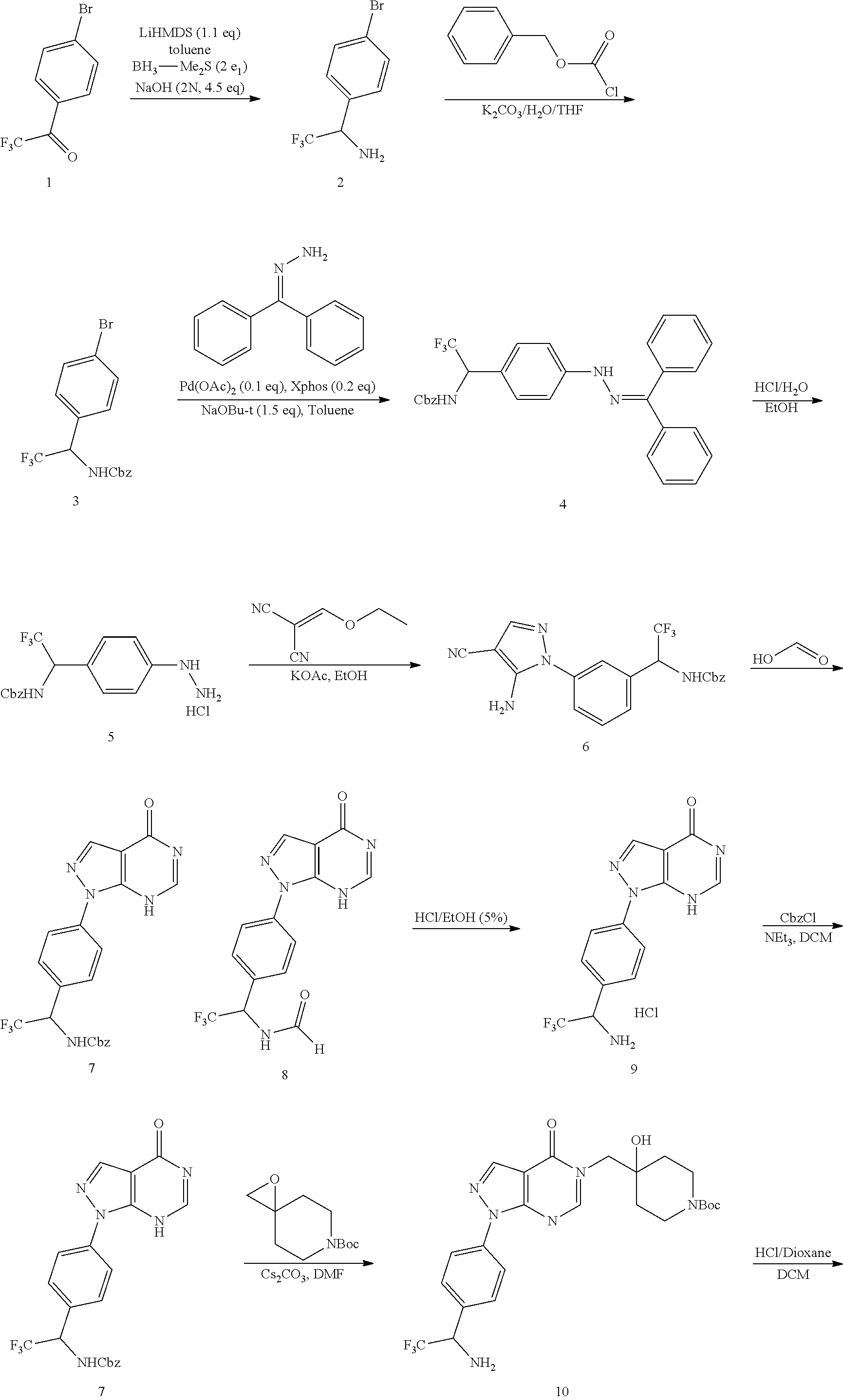

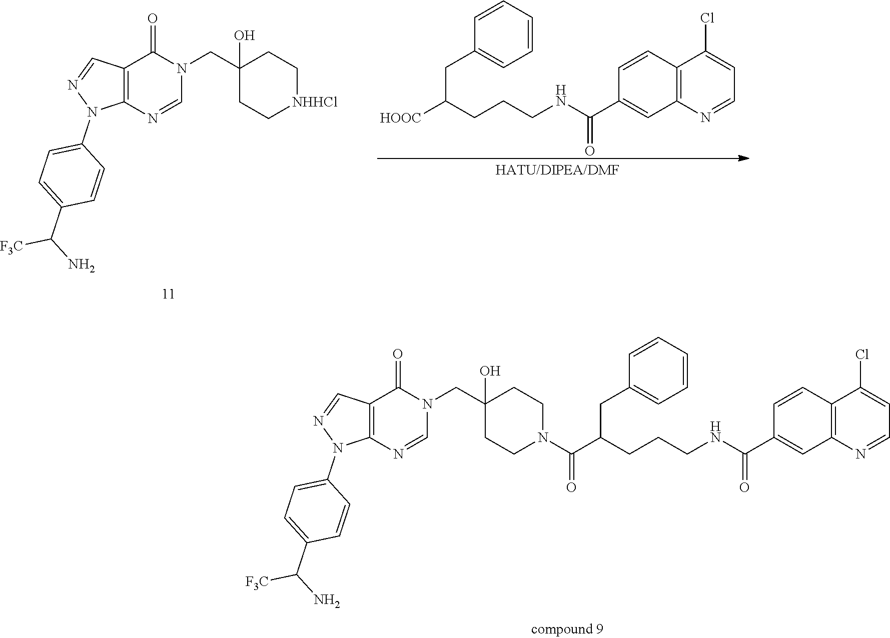

### 1-(4-bromophenyl)-2,2,2-trifluoroethanamine (Intermediate 2)

To a mixture of 1-(4-bromophenyl)-2,2,2-trifluoroethanone (2530 mg, 10.0 mmol) in toluene (60 mL) was added dropwise LiHMDS (11 mL, 11 mmol). After stirred at rt for 15 min, borane-THF (10 mL, 20 mmol) was added. The resulting mixture was stirred at rt for 20 min, cooled down to 0° C., and then 2 N NaOH solution (23 mL) was added. After stirred at rt for 90 min the mixture was diluted with EtOAc (100 mL), the organic layer was separated, washed with 2 N NaOH solution (100 mL), water (100 mL) and brine (100 mL), dried over anhydrous Na₂SO₄, concentrated and purified by column chromatography (10% EtOAc in hexane) to give intermediate 2 (2.22 g, Yield 87.4) as oil. LCMS: (m/z) 254.0, 256.0 [M+H]⁺

### benzyl 1-(4-bromophenyl)-2,2,2-trifluoroethylcarbamate (Intermediate 3)

To a solution of intermediate 2 (1967 mg, 7.74 mmol) in THF (25 mL) was added a solution of K₂CO₃ (1451 mg, 8.51 mmol) in water (25 mL) at 0° C., and then benzyl carbonochloridate (1451 mg, 8.51 mmol) was added dropwise. The mixture was stirred at rt for 16 hours. Brine (20 mL) and EtOAc (50 mL) were added to the mixture, and the phases were separated. The aqueous phase was extracted with EtOAc (40 mL×2). The combined organic phase was dried over magnesium sulfate, filtered and concentrated in vacuum. The residue was purified by flash column chromatograph (PE/EtOAc=10/1) to give intermediate 3 (2.05 g, Yield 68.6%) as oil. LCMS: (m/z) 410.0 [M+Na]⁺

### benzyl 1-(4-(2-(diphenylmethylene)hydrazinyl)phenyl)-2,2,2-trifluoroethylcarbamate (Intermediate 4)

Under an argon atmosphere, a solution of benzyl 1-(4-bromophenyl)-2,2,2-trifluoroethylcarbamate (2.126 g, 5.48 mmol) in toluene (20 mL) was added to a mixture of (diphenylmethylene)hydrazine (1.129 g, 6.58 mmol), sodium-tert-butoxide (790 mg, 8.22 mmol), palladium(II)acetate (123 mg, 0.55 mmol) and X-phos (524 mg, 1.1 mmol) in toluene (30 mL). The reaction mixture was stirred at 90° C. for 16 hours. After cooled down to rt the mixture was diluted with EtOAc (200 mL), washed with brine (200 mL), dried over anhydrous Na₂SO₄, concentrated and purified by flash column chromatograph (DCM/MeOH=30/1) to give intermediate 4 (1.575 g, Yield 52.3%) as a pale yellow solid. LCMS: (m/z) 504.2 [M+H]⁺

### benzyl 2,2,2-trifluoro-1-(4-hydrazinylphenyl)ethylcarbamate hydrochloride (Intermediate 5)

To a mixture of benzyl 1-(4-(2-(diphenylmethylene)hydrazinyl)phenyl)-2,2,2-trifluoroethylcarbamate (805 mg, 1.6 mmol) in EtOH (32 mL) was added concentrated HCl solution (8.0 mL, 96.0 mmol). The mixture was stirred at 45° C. for 4 hours. After cooled down to rt the mixture was diluted with H₂O (40 mL) and extracted with DCM (3×50 mL). The combined organics were extracted with H₂O (2×25 mL), the combined aqueous were concentrated and lyophilized to give intermediate 5 (300 mg) as pale brown solid. LCMS: (m/z) 340.1 [M+H]⁺

### benzyl 1-(3-(5-amino-4-cyano-1H-pyrazol-1-yl)phenyl)-2,2,2-trifluoroethylcarbamate (Intermediate 6)

A mixture of benzyl 2,2,2-trifluoro-1-(4-hydrazinylphenyl)ethylcarbamate hydrochloride (350 mg, 0.93 mmol), 2-(ethoxymethylene)malononitrile (114 mg, 0.93 mmol) and KOAc (91 mg, 0.93 mmol) in EtOH (20 mL) was stirred at 80° C. for 2 hours. After cooled down to rt the mixture was filtered and washed with EtOH (10 mL). The combined filtrate and washing was concentrated to leave crude intermediate 6 (330 mg, Yield 80%) as pale yellow solid. LCMS: (m/z) 416.1 [M+H]⁺

### benzyl 2,2,2-trifluoro-1-(4-(4-oxo-4H-pyrazolo[3,4-d]pyrimidin-1(7H)-yl)phenyl)ethylcarbamate (7) and N-(2,2,2-trifluoro-1-(4-(4-oxo-4H-pyrazolo[3,4-d]pyrimidin-1(7H)-yl)phenyl)ethyl)formamide (Intermediate 8)

A mixture of benzyl 1-(3-(5-amino-4-cyano-1H-pyrazol-1-yl)phenyl)-2,2,2-trifluoroethylcarbamate (330 mg, 0.79 mmol) in HCOOH (10 mL) was stirred at 100° C. for 16 hours. After cooled down to rt the mixture the mixture was poured into ice water (30 mL) and extracted with EtOAc (3×50 mL). The combined organics were washed with brine (100 mL), dried over Na₂SO₄, filtered and concentrated to give a mixture of intermediate 7 (26% in LCMS) and 8 (60% in LCMS) (totally 270 mg) as a pale yellow solid, which was used directly in the next step. LCMS: 7: (m/z) 441.3 [M+H]⁺, 8: (m/z) 338.1 [M+H]⁺

### 1-(4-(1-amino-2,2,2-trifluoroethyl)phenyl)-1H-pyrazolo[3,4-d]pyrimidin-4(7H)-one (Intermediate 9)

The mixture of compound 7 and 8 (243 mg, 0.72 mmol) in HCl/EtOH (5%, 10 mL) was stirred at 80° C. for 2 hours. The mixture was concentrated to give intermediate 9 as brown solid (260 mg). LCMS: (m/z) 310.1 [M+H]⁺

### benzyl 2,2,2-trifluoro-1-(4-(4-oxo-4H-pyrazolo[3,4-d]pyrimidin-1(7H)-yl)phenyl)ethylcarbamate (Intermediate 7)

To a stirred mixture of 1-(4-(1-amino-2,2,2-trifluoroethyl)phenyl)-1H-pyrazolo[3,4-d]pyrimidin-4(7H)-one hydrochloride (260 mg, 0.84 mmol) and NEt₃ (255 mg, 2.521 mmol) in DCM (20 mL) was added a solution of CbzCl (172 mg, 1.00 mmol). The mixture was stirred at rt for 16 hours, diluted with DCM (100 mL), washed with aq. NaHCO₃ solution (50 mL) and brine (50 mL), dried over anhydrous Na₂SO₄, concentrated and purified by flash column chromatograph (DCM/MeOH=30/1) to give intermediate 7 (158 mg, Yield 47%) as a brown solid. LCMS: (m/z) 444.1 [M+H]⁺

### tert-butyl 4-((1-(4-(1-amino-2,2,2-trifluoroethyl)phenyl)-4-oxo-1H-pyrazolo[3,4-d]pyrimidin-5(4H)-yl)methyl)-4-hydroxypiperidine-1-carboxylate (Intermediate 10)

To a stirred mixture of benzyl 2,2,2-trifluoro-1-(4-(4-oxo-4H-pyrazolo[3,4-d]pyrimidin-1(7H)-yl)phenyl)ethylcarbamate (136 mg, 0.306 mmol) and 1-oxa-6-azaspiro[2.5]octane-6-carboxylic acid, 1,1-dimethylethyl ester (85 mg, 0.398 mmol) in DMF (10 mL) was added Cs₂CO₃ (150 mg, 0.459 mmol). The mixture was stirred at 90° C. for 7 hours. After cooled down to rt the mixture was diluted with H₂O (30 mL) and extracted with EtOAc (3×30 mL), the combined organic was dried over anhydrous Na₂SO₄, concentrated and purified by prep-HPLC (C18 column, CH₃CN/H₂O, containing 0.05% NH₄HCO₃) to give intermediate 10 (46.9 mg, Yield 25.2%) as a white solid. LCMS: (m/z) 545.3 [M+Na]⁺

### 1-(4-(1-amino-2,2,2-trifluoroethyl)phenyl)-5-((4-hydroxypiperidin-4-yl)methyl)-1H-pyrazolo[3,4-d]pyrimidin-4(5H)-one hydrochloride (Intermediate 11)

To a mixture of tert-butyl 4-((1-(4-(1-amino-2,2,2-trifluoroethyl)phenyl)-4-oxo-1H-pyrazolo[3,4-d]pyrimidin-5(4H)-yl)methyl)-4-hydroxypiperidine-1-carboxylate (46.9 mg, 0.09 mmol) in DCM (5 mL) was added HCl solution in 1,4-dioxane (4 M, 0.45 mL, 1.8 mmol) at 0° C. The mixture was stirred at rt for 4 hours and concentrated in vacuum to give intermediate 11 (42 mg, Yield 100%) as a white solid. LCMS: (m/z) 423.1 [M+H]⁺,

### N-(5-(4-((1-(4-(1-amino-2,2,2-trifluoroethyl)phenyl)-4-oxo-1H-pyrazolo[3,4-d]pyrimidin-5(4H)-yl)methyl)-4-hydroxypiperidin-1-yl)-4-benzyl-5-oxopentyl)-4-chloroquinoline-7-carboxamide (Compound 9)

A mixture of 1-(4-(1-amino-2,2,2-trifluoroethyl)phenyl)-5-((4-hydroxypiperidin-4-yl)methyl)-1H-pyrazolo[3,4-d]pyrimidin-4(5H)-one hydrochloride (42 mg, 0.091 mmol), 2-benzyl-5-(4-chloroquinoline-7-carboxamido)pentanoic acid (28 mg, 0.07 mmol), HATU (29 mg, 0.077 mmol) and DIPEA (36 mg, 0.091 mmol) in DMF (5 mL) was stirred at rt for 1 h. The mixture was purified directly by prep-HPLC (C18 column, CH₃CN/H₂O, containing 0.05% NH₄HCO₃) to give compound 9 (18.2 mg, Yield 24.8%) as a white solid. ¹H NMR (400 MHz, DMSO-d₆) δ (ppm) 8.96-8.84 (m, 2H), 8.61-8.58 (m, 1H), 8.39-8.35 (m, 1H), 8.31-8.24 (m, 2H), 8.20-8.13 (m, 1H), 8.09-8.01 (m, 2H), 7.86 (d, 4.8 Hz), 7.70-7.68 (m, 2H), 7.29-7.09 (m, 5H), 4.84 (s, 1H), 4.65-4.54 (m, 1H), 4.18-3.56 (m, 4H), 3.19-3.08 (m, 2H), 2.88-2.55 (m, 6H), 1.70-1.04 (m, 8H). LCMS: (m/z) 801.3 [M+H]⁺

### Synthesis of Compound 10

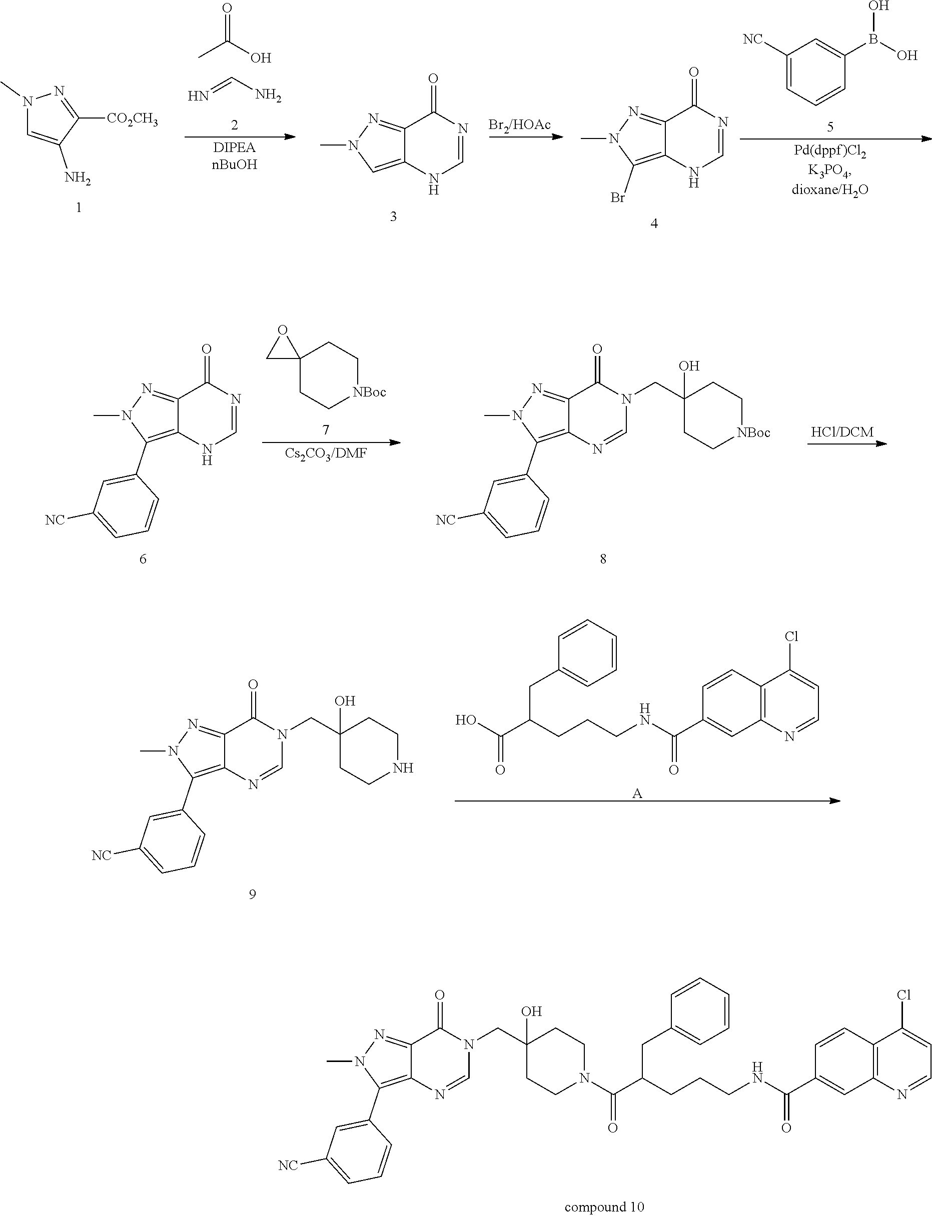

### 1-(4-nitrophenyl)-1,5-dihydro-4H-pyrazolo[3,4-d]pyrimidin-4-one (Intermediate 3)

A mixture of methyl 4-amino-1-methyl-1H-pyrazole-3-carboxylate (500 mg, 3.2 mmol), formimidamide acetate (370 mg, 3.5 mmol) and DIPEA (3.0 mL) in n-BuOH (4.0 mL) was stirred at 110° C. for 1 h. After cooled down to rt the mixture was filtered to get intermediate 3 (450 mg, yield 93%) as off-white solid. LCMS (m/z): 151.1 [M+H]⁺.

### 3-bromo-2-methyl-2,4-dihydro-7H-pyrazolo[4,3-d]pyrimidin-7-one (Intermediate 4)

A mixture of intermediate 3 (400 mg, 2.6 mmol) and Br₂ (853 mg, 5.2 mmol) in AcOH (15.0 mL) was stirred at 95° C. for 16 h. After cooled down to rt the mixture was filtered to get the crude compound 4 (800 mg) as yellow solid. LCMS (m/z): 229.1 [M+H]⁺.

### 3-(2-methyl-7-oxo-4,7-dihydro-2H-pyrazolo[4,3-d]pyrimidin-3-yl)benzonitrile (Intermediate 6)

A mixture of compound 4 (700 mg, 3.0 mmol), (3-cyanophenyl)boronic acid (1100 mg, 7.5 mmol), K₃PO₄ (1900 mg, 9.0 mmol) and Pd(dppf)Cl₂ (220 mg, 0.3 mmol) in 1,4-dioxane (20.0 mL) and H₂O (4.0 mL) was heated in a microwave reactor at 150° C. for 40 min, the mixture was diluted with water (50 mL) and extracted with EtOAc (50 mL×2), the combined organic was dried over anhydrous Na₂SO₄, filtered, concentrated and purified by column chromatography on silica-gel (MeOH/DCM=1/30) to get intermediate 6 (300 mg, yield 40%) as white solid. LCMS (m/z): 252.2 [M+H]⁺.

### tert-butyl 4-((3-(3-cyanophenyl)-2-methyl-7-oxo-2,7-dihydro-6H-pyrazolo[4,3-d]pyrimidin-6-yl)methyl)-4-hydroxypiperidine-1-carboxylate (Intermediate 8)

A mixture of intermediate 6 (125 mg, 0.5 mmol), tert-butyl 1-oxa-6-azaspiro[2.5]octane-6-carboxylate (127 mg, 0.6 mmol) and Cs₂CO₃ (244 mg, 0.75 mmol) in DMF (10.0 mL) was stirred at 80° C. for 16 h. After cooled down to rt the mixture was diluted with EtOAc (100 mL), washed with water (20 mL*4), dried over anhydrous Na₂SO₄, filtered, concentrated and purified by column chromatography on silica-gel (EA/PE=1/2) to get intermediate 8 (150 mg, yield 64%) as yellow solid. LCMS (m/z): 409.2 [M-55+H]⁺.

### 3-(6-((4-hydroxypiperidin-4-yl)methyl)-2-methyl-7-oxo-6,7-dihydro-2H-pyrazolo[4,3-d]pyrimidin-3-yl)benzonitrile (Intermediate 9)

To the mixture of intermediate 8 (92.8 mg, 0.2 mmol) in DCM (4.0 mL) was added HCl solution in 1,4-dioxane (4 M, 1.0 mL), the mixture was stirred at rt for 2 h and concentrated in vacuum to leave crude intermediate 9 (80 mg) as oil. LCMS (m/z): 365.2 [M+H]⁺.

### N-(4-benzyl-5-(4-((3-(3-cyanophenyl)-2-methyl-7-oxo-2,7-dihydro-6H-pyrazolo[4,3-d]pyrimidin-6-yl)methyl)-4-hydroxypiperidin-1-yl)-5-oxopentyl)-4-chloroquinoline-7-carboxamide (Compound 10)

A mixture of compound A (79.2 mg, 0.2 mmol), HATU (83.6 mg, 0.22 mmol) and DIPEA (0.2 mL, 1.0 mmol) in DCM (10.0 mL) was stirred at rt for 30 min, the mixture was added to the solution of intermediate 9 (72.8 mg, 0.2 mmol) and DIPEA (0.1 mL, 0.2 mmol) in DCM (10.0 mL), the resulting mixture was stirred at rt for 2 h, diluted with DCM (20 mL), washed with water (20 mL), dried over anhydrous Na₂SO₄, filtered, concentrated and purified by column chromatography on silica-gel (MeOH/DCM=1/20) and prep-HPLC (C18 column, CH₃CN/H₂O, containing 0.05% NH₄HCO₃) to afford compound 10 as white solid (43.3 mg, yield 29%). ¹H NMR (DMSO-d₆, 400 MHz): δ (ppm) 8.93-8.88 (m, 2H), 8.61-8.58 (m, 1H), 8.30-8.26 (m, 1H), 8.21-8.15 (m, 2H), 8.09-8.05 (m, 1H), 8.01-7.95 (m, 2H), 7.86 (d, J=4.8 Hz, 1H), 7.82-7.78 (m, 1H), 7.29-7.12 (m, 5H), 4.80 (s, 1H), 4.17-4.15 (m, 4H), 3.95-3.60 (m, 3H), 3.28-3.16 (m, 3H), 3.13-2.67 (m, 4H), 1.65-1.09 (m, 8H). LCMS (m/z): 743.3 [M+H]⁺

### Synthesis of Compound 11, 12, and 13

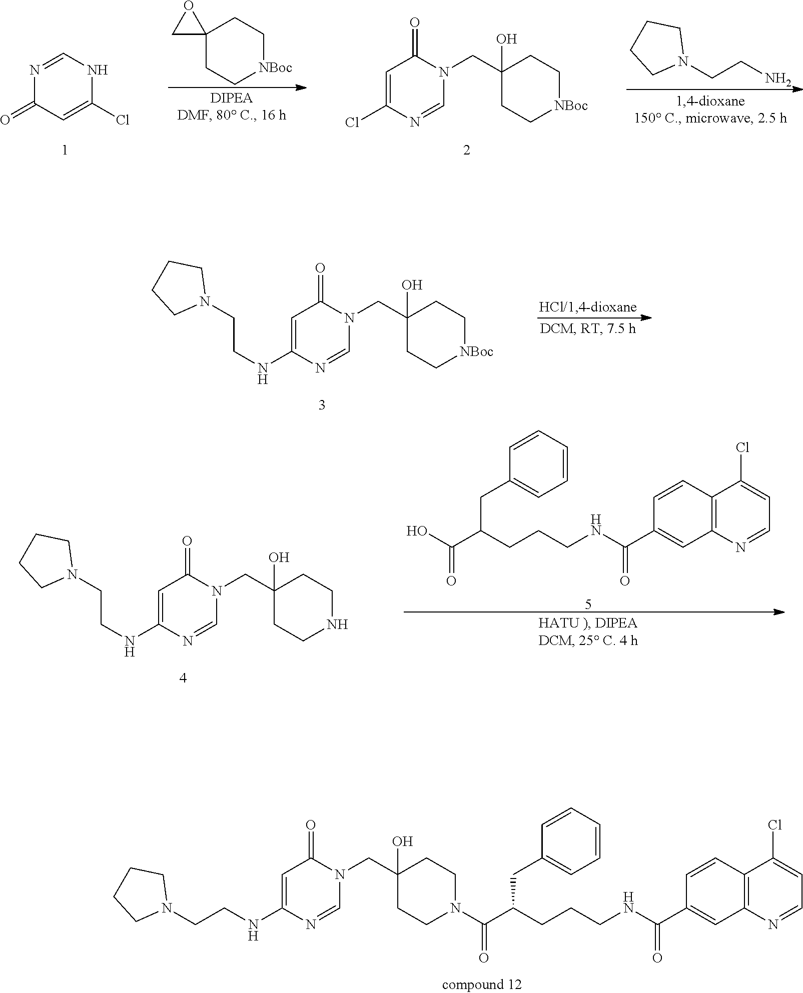

### tert-butyl 4-((4-chloro-6-oxopyrimidin-1(6H)-yl)methyl)-4-hydroxypiperidine-1-carboxylate (Intermediate 2)

The mixture of tert-butyl 1-oxa-6-azaspiro[2.5]octane-6-carboxylate (5026 mg, 23.6 mmol), 6-chloropyrimidin-4(1H)-one (1536 mg, 11.8 mmol) and DIEA (3 mL) in DMF (50 mL) was stirred at 80° C. over night. The mixture was diluted with EtOAc (100 mL), washed with water (100 mL) and brine (100 mL), dried over anhydrous Na₂SO₄, concentrated and purified by column chromatography on silica gel (EA/PE=1/2) to afford intermediate 2 (2.7 g, yield 66%) as a white solid. LCMS: (m/z) 366 [M+Na]⁺

### tert-butyl 4-hydroxy-4-((6-oxo-4-((2-(pyrrolidin-1-yl)ethyl)amino)pyrimidin-1(6H)-yl)methyl)piperidine-1-carboxylate (Intermediate 3)

The mixture of compound 2 (498 mg, 1.45 mmol) and 2-(pyrrolidin-1-yl)ethan-1-amine (550 mg, 4.8 mmol) in 1,4-dioxane (10 mL) was heated in a microwave reactor at 150° C. for 2.5 hours. The reaction mixture was concentrated and purified by HPLC to afford intermediate 3 (200 mg, yield 32.6%) as a white solid. LCMS: (m/z) 422 [M+H]⁺

### 3-((4-hydroxypiperidin-4-yl)methyl)-6-((2-(pyrrolidin-1-yl)ethyl)amino)pyrimidin-4(3H)-one (Intermediate 4)

To the mixture of compound 3 (200 mg, 0.47 mmol) in DCM (4 mL) was added HCl-dioxane (2 mL), the mixture was stirred at rt for 2 h and concentrated to leave crude intermediate 4 (250 mg) as a yellow solid. LCMS (m/z): 322 [M+H]⁺

### N-(4-benzyl-5-(4-hydroxy-4-((6-oxo-4-(2-(pyrrolidin-1-yl)ethylamino)pyrimidin-1(6H)-yl)methyl)piperidin-1-yl)-5-oxopentyl)-4-chloroquinoline-7-carboxamide (compounds 11, 12, 13)

The mixture of intermediate 4 (180 mg, 0.5 mmol), intermediate 5 (200 mg, 0.5 mmol), DIEA (0.5 mL) and HATU (198 mg, 0.55 mmol) in DMF (4 mL) was stirred at rt for 30 minutes. The reaction mixture was purified directly by prep-HPLC (C18 column, CH₃CN/H₂O, containing 0.050% NH₄HCO₃) to afford 100 mg of compound 11, which was separated by chiral HPLC to afford two enantiomers: compound 12 (isomer 1: 29.6 mg) as a white solid and compound 13 (isomer 2: 24 mg) as a white solid.

### Compound 12:

¹H NMR (400 MHz, DMSO-d₆) δ (ppm) 8.99-8.81 (m, 2H), 8.59 (d, J=4.8 Hz, 1H), 8.28 (dd, J=8.7, 2.0 Hz, 1H), 8.22-8.13 (m, 1H), 7.90 (dd, J=30.3, 14.0 Hz, 2H), 7.31-7.07 (m, 5H), 6.96 (s, 1H), 5.08 (s, 1H), 4.11-3.94 (m, 1H), 3.75-3.65 (m, 3H), 3.31-3.13 (m, 6H), 2.73 (ddd, J=26.2, 24.9, 16.8 Hz, 9H), 1.78-1.39 (m, 9H), 1.20 (ddd, J=53.0, 32.7, 12.5 Hz, 4H). LC-MS: (m/z) 700 [M+H]⁺

### Compound 13:

¹H NMR (400 MHz, DMSO-d₆) δ (ppm) 8.99-8.81 (m, 2H), 8.59 (d, J=4.8 Hz, 1H), 8.28 (dd, J=8.7, 2.0 Hz, 1H), 8.22-8.13 (m, 1H), 7.90 (dd, J=30.3, 14.0 Hz, 2H), 7.31-7.07 (m, 5H), 6.96 (s, 1H), 5.08 (s, 1H), 4.11-3.94 (m, 1H), 3.75-3.65 (m, 3H), 3.31-3.13 (m, 6H), 2.73 (ddd, J=26.2, 24.9, 16.8 Hz, 9H), 1.80-1.60 (m, 5H), 1.50 (s, 3H), 1.38-1.00 (m, 4H). LC-MS: (m/z) 700 [M+H]⁺

### Synthesis of Compound 14

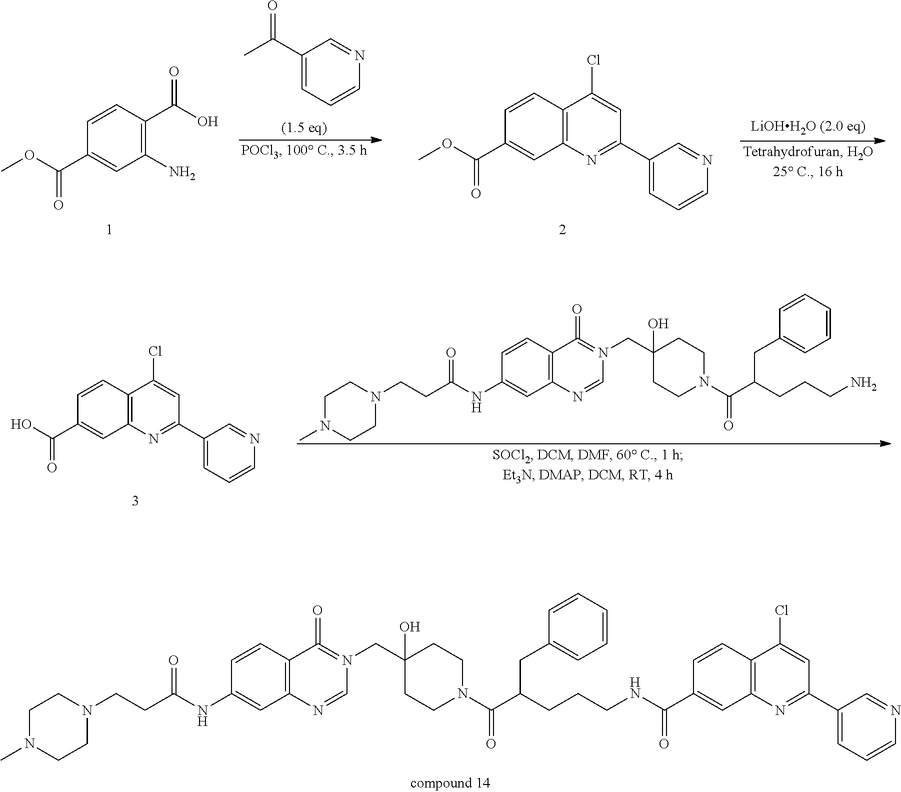

### methyl 4-chloro-2-(pyridin-3-yl)quinoline-7-carboxylate (Intermediate 2)

A mixture of 2-amino-4-(methoxycarbonyl)benzoic acid (506.1 mg, 2.593 mmol) and 1-(pyridin-3-yl)ethanone (322.4 mg, 2.661 mmol) in POCl₃ (8 mL) was stirred at 100° C. for 3.5 hours. Iced water (50 mL) was poured into the reaction mixture while stirring. Saturated Na₂CO₃ solution was added into the solution to adjust to pH 8. DCM (50 mL) was added into the solution and the layers were separated. The aqueous phase was extracted with DCM (40 mL×6), the combined organic phase was washed with saturated NaCl solution (100 mL), dried over anhydrous Na₂SO₄, filtered, concentrated and purified by flash column chromatography (EA/PE=1/4) to afford intermediate 2 (0.260 g, yield 33.63%) as yellow solid. LCMS: (m/z) 299.1 [M+H]⁺

### 4-chloro-2-(pyridin-3-yl)quinoline-7-carboxylic acid (Intermediate 3)

A mixture of methyl 4-chloro-2-(pyridin-3-yl)quinoline-7-carboxylate (260 mg, 0.872 mmol, 1.0 eq) and LiOH·H₂O (74.9 mg, 1.785 mmol, 2.0 eq) in tetrahydrofuran (10 mL) and water (3 mL) was stirred at 25° C. for 16 hours. The reaction mixture was concentrated under vacuum, the residue was diluted with water (50 mL), adjusted to ph 4 with diluted HCl solution and then lyophilized overnight to afford intermediate 3 (180 mg, 72.68% yield) as yellow solid. LCMS: (m/z) 285.0 [M+H]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(pyridin-3-yl)quinoline-7-carboxamide (Compound 14)

SOCl₂ (76 μL, 0.8 eq) was added into a solution of 4-chloro-2-(pyridin-3-yl)quinoline-7-carboxylic acid (0.1857 g, 0.654 mmol, 1.0 eq) in anhydrate dichloromethane (10 mL) and N,N-dimethylformamide (0.1 mL) while stirring in an ice-water bath. The mixture was stirred at 60° C. for 1 hour, and then concentrated in vacuum, the residue was dissolved in anhydrate dichloromethane (25 mL), added dropwise into a solution of N-(3-((1-(5-amino-2-benzylpentanoyl)-4-hydroxypiperidin-4-yl)methyl)-4-oxo-3,4-dihydroquinazolin-7-yl)-3-(4-methylpiperazin-1-yl)propanamide (225.2 mg, 0.365 mmol, 0.6 eq), Et₃N (0.2 mL, 8.0 eq) and DMAP (4.5 mg, 0.037 mmol, 0.1 eq) in anhydrate dichloromethane (15 mL) while stirring in an ice-water bath. The resulting mixture was stirred at room temperature for 4 hours, concentrated and purified by prep-HPLC to afford the target compound 14 (14.9 mg, 2.58% yield) as white solid. ¹H NMR (400 MHz, DMSO-d₆) δ (ppm) 10.55 (d, J=6.9 Hz, 1H), 9.49 (s, 1H), 9.00-8.94 (m, 1H), 8.80-8.60 (m, 4H), 8.32-8.0 (m, 5H), 7.65-7.57 (m, 2H), 7.28-7.10 (m, 5H), 4.87 (s, 1H), 4.17-4.01 (m, 1H), 3.95-3.49 (m, 3H), 3.45-2.65 (m, 13H), 2.60-2.10 (m, 9H), 1.75-1.02 (m, 8H). LC-MS: (m/z) 884.3 [M+H]⁺

### Synthesis of Compound 15

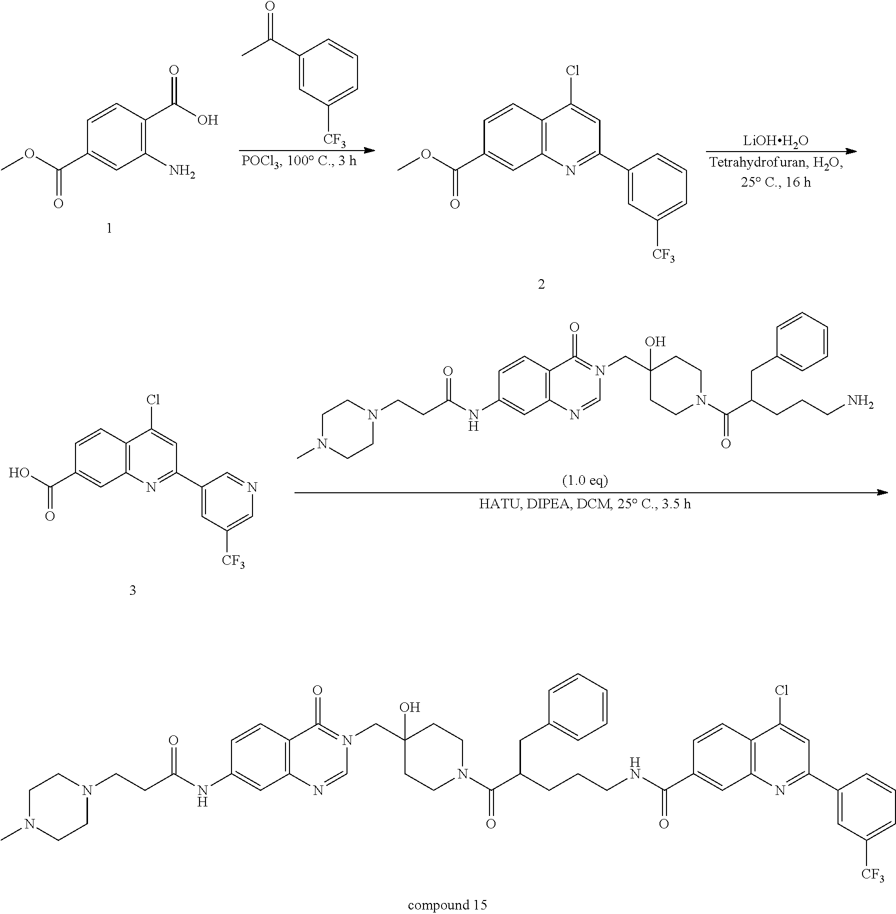

### methyl 4-chloro-2-(3-(trifluoromethyl)phenyl)quinoline-7-carboxylate (Intermediate 2)

A mixture of 2-amino-4-(methoxycarbonyl)benzoic acid (344.3 mg, 1.7652 mmol, 1.0 eq) and 1-(3-(trifluoromethyl)phenyl)ethanone (504.3 mg, 2.6819 mmol, 1.5 eq) in POCl₃ (6 mL) was stirred at 100° C. for 3 hours. The reaction mixture was concentrated in vacuum, iced water was added while stirring, the mixture was adjusted to pH 8 with solid sodium bicarbonate and saturated sodium carbonate solution. Dichloromethane (50 mL) was added and layers were separated. The aqueous phase was washed with dichloromethane (40 mL×3), the combined organic phase was washed with saturated NaCl solution (100 mL), dried over anhydrous Na₂SO₄, filtered, concentrated and purified by flash column chromatography (EA/PE=1/5) to afford intermediate 2 (250 mg, 0.685 mmol, yield 38.80%) as white solid. LCMS (m/z): 366.0 [M+H]⁺

### 4-chloro-2-(3-(trifluoromethyl)phenyl)quinoline-7-carboxylic acid (Intermediate 3)

A mixture of methyl 4-chloro-2-(3-(trifluoromethyl)phenyl)quinoline-7-carboxylate (250 mg, 0.685 mmol, 1.0 eq) and LiOH H₂O (56.4 mg, 1.344 mmol, 2.0 eq) in tetradrofuran (10 mL) and H₂O (3 mL) was stirred at 25° C. for 16 hours. The reaction mixture was concentrated in vacuum, diluted with water (50 mL) and adjusted to pH-5 with diluted HCl solution. The mixture was extracted with ethyl acetate (50 mL×3), the combined organic phase was washed with saturated NaCl solution (100 mL), dried over anhydrous Na₂SO₄, filtered and concentrated in vacuum to leave the crude intermediate 3 (173 mg, 0.493 mmol, 71.95% yield) as white solid. LC-MS (m/z): 352.0 [M+H]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(3-(trifluoromethyl)phenyl)quinoline-7-carboxamide (Compound 15)

A mixture of 4-chloro-2-(3-(trifluoromethyl)phenyl)quinoline-7-carboxylic acid (32 mg, 0.091 mmol, 1.0 eq), DIPEA (75.8 mg, 0.587 mmol, 5.0 eq) and HATU (41.6 mg, 0.109 mmol, 1.2 eq) in dichloromethane (2 mL) was stirred at 25° C. for 30 minutes, and then N-(3-((1-(5-amino-2-benzylpentanoyl)-4-hydroxypiperidin-4-yl)methyl)-4-oxo-3,4-dihydroquinazolin-7-yl)-3-(4-methylpiperazin-1-yl)propanamide (57.1 mg, 0.093 mmol, 1.0 eq) was added. The resulting mixture was stirred at 25° C. for 3.5 hours and diluted with dichloromethane (10 mL) and water (50 mL). Layers was separated and the aqueous phase was extracted with dichloromethane (50 mL×3). The combined organic was dried over anhydrous Na₂SO₄, filtered, concentrated and purified by prep-HPLC to afford the target compound 15 (27.9 mg, 32.26% yield) as white solid. ¹H NMR (400 MHz, DMSO-d₆) δ (ppm) 10.52 (d, J=7.6 Hz, 1H), 8.95-8.90 (m, 1H), 8.73-8.62 (m, 4H), 8.31-8.27 (m, 1H), 8.22-8.16 (m, 1H), 8.12-7.98 (m, 3H), 7.93-7.89 (m, 1H), 7.85-7.80 (m, 1H), 7.64-7.56 (m, 1H), 7.26-7.10 (m, 5H), 4.85-4.84 (m, 1H), 4.16-3.60 (m, 5H), 3.20-3.10 (m, 2H), 2.85-2.60 (m, 7H), 2.45-2.20 (m, 7H), 1.75-0.95 (m, 10H). LC-MS (m/z): 951.3 [M+H]⁺

### Synthesis of Compound 16

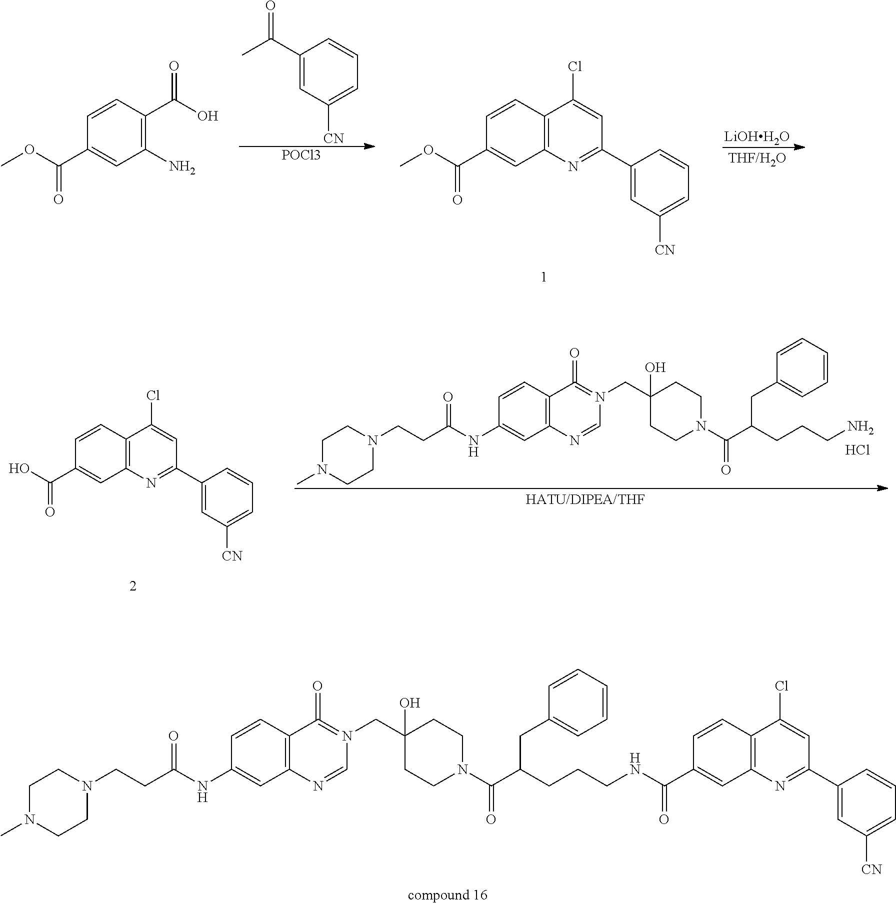

### methyl 4-chloro-2-(3-cyanophenyl)quinoline-7-carboxylate (Intermediate 1)

A mixture of 2-amino-4-(methoxycarbonyl)benzoic acid (558 mg, 2.86 mmol) and 3-acetylbenzonitrile (623 mg, 4.29 mmol) in POCl₃ (10 mL) was stirred at 100° C. for 3 hour, after cooled down to rt, the mixture was concentrated, the residue was taken into ice water (20 mL), adjusted to pH 8-9 with solid NaHCO₃ and extracted with DCM (50 mL×3). The combined organic was dried over anhydrous Na₂SO₄, concentrated and purified by flash column chromatograph (DCM/MeOH=40/1) to give intermediate 1 (245 mg, Yield 11%) as pale yellow solid. LCMS: (m/z) 323.1 [M+H]⁺,

### 4-chloro-2-(3-cyanophenyl)quinoline-7-carboxylic acid (Intermediate 2)

To a solution of methyl 4-chloro-2-(3-cyanophenyl)quinoline-7-carboxylate (245 mg, 0.759 mmol) in THF (10 mL) was added a solution of LiOH·H₂O (48 mg, 1.138 mmol) in H₂O (5 mL). The mixture was stirred at rt for 16 hours and then concentrated in vacuum. The residue was diluted with water (20 mL) and acidified with 1 M HCl solution (pH 4-5), the resulting mixture was extracted with ethyl acetate (3×50 mL). The combined organic phase was dried and concentrated in vacuum to leave crude intermediate 2 (120 mg, Yield 51.3%) as a yellow solid. LCMS: (m/z) 309.1 [M+Na]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(3-cyanophenyl)quinoline-7-carboxamide (Compound 16)

A mixture of intermediate 2 (62 mg, 0.2 mmol), SM-2 (124 mg, 0.2 mmol), HATU (84 mg, 0.22 mmol) and DIEA (78 mg, 0.6 mmol) in THF (5 mL) was stirred at rt for 2 hours. The mixture was concentrated and purified by flash column chromatograph (DCM/MeOH=10/1) and prep-HPLC (C18 column, CH₃CN/H₂O, containing 0.05% NH₄HCO₃) to give compound 16 (24 mg, Yield 13%) as a white solid. ¹H NMR (500 MHz, DMSO-d₆) δ (ppm) 10.52 (d, J=9.5 Hz, 1H), 8.98-8.89 (m, 1H), 8.79 (d, J=9.0 Hz, 1H), 8.70 (d, J=8.5 Hz, 1H), 8.68-8.62 (m, 2H), 8.31-8.27 (m, 1H), 8.21-8.14 (m, 1H), 8.12-7.97 (m, 4H), 7.82-7.75 (m, 1H), 7.63-7.55 (m, 1H), 7.26-7.08 (m, 5H), 4.84 (d, J=8.0 Hz, 1H), 4.18-3.98 (m, 1H), 3.90-3.58 (m, 3H), 3.32-3.09 (m, 4H), 2.91-2.52 (m, 9H), 2.47-2.18 (m, 6H), 2.14 (s, 3H), 1.70-1.02 (m, 8H). LCMS: (m/z) 908.3 [M+H]⁺

### Synthesis of Compound 17

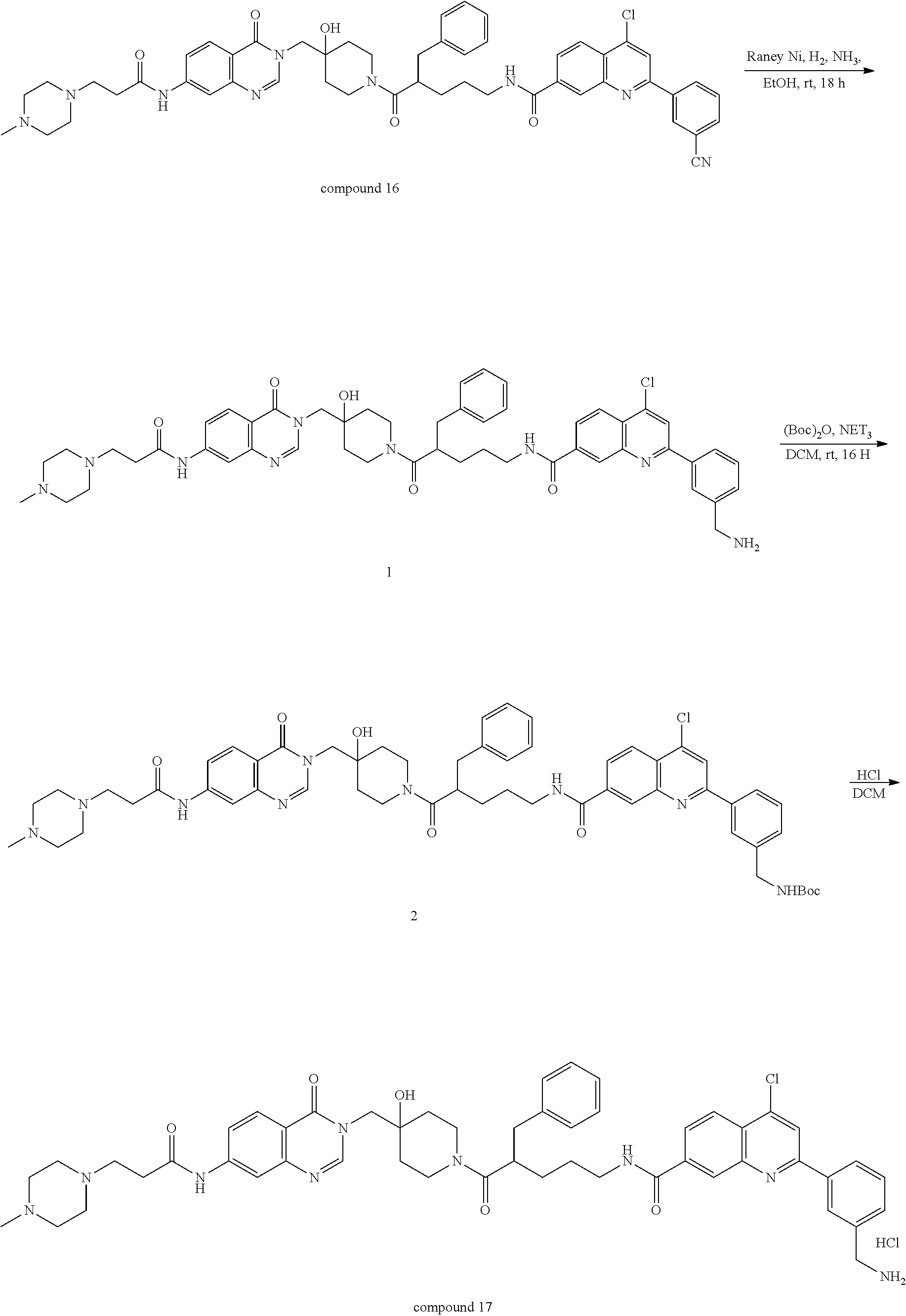 [SMILES: CN1CCN(CCC(=O)Nc2ccc3c(=O)n(CC4(O)CCN(C(=O)C(CCCNC(=O)c5ccc6c(Cl)cc(-c7cccc(C#N)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7cccc(CN)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7cccc(CNC(=O)OC(C)(C)C)c7)nc6c5)Cc5ccccc5)CC4)cnc3c2)CC1 ; Cl ; CN1CCN(CCC(=O)Nc2ccc3c(=O)n(CC4(O)CCN(C(=O)C(CCCNC(=O)c5ccc6c(Cl)cc(-c7cccc(CN)c7)nc6c5)Cc5ccccc5)CC4)cnc3c2)CC1 ; Cl]

### 2-(3-(aminomethyl)phenyl)-N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (Intermediate 1)

To a mixture of compound 16 (239 mg, 0.263 mmol) in EtOH (40 mL) was added Raney Ni (309 mg, 5.26 mmol) and NH₃ solution in CH₃OH (7M, 5 mL). The mixture was stirred at 50° C. under H₂ (1 atm) for 16 hours. The mixture was cooled down to rt and filtered, the cake was washed with EtOH (20 mL), the combined filtrate and washing was concentrated to leave crude intermediate 1 (254 mg) as grey solid. LCMS: (m/z) 912.3 [M+H]⁺,

### tert-butyl 3-(7-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentylcarbamoyl)-4-chloroquinolin-2-yl)benzylcarbamate (Intermediate 2)

To a mixture of 2-(3-(aminomethyl)phenyl)-N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (254 mg, 0.278 mmol) and TEA (56 mg, 0.556 mmol) in DCM (20 mL) was added (Boc)₂O (67 mg, 0.306 mmol) at rt. The mixture was stirred at rt for 16 hours, diluted with DCM (100 mL), washed with aq. NaHCO₃ solution (100 mL) and brine (100 mL), dried over anhydrous Na₂SO₄, concentrated and purified by prep-HPLC (C18 column, CH₃CN/H₂O, containing 0.05% NH₄HCO₃) to give intermediate 2 (48 mg, yield 17%) as a white solid. LCMS: (m/z) 1012.5 [M+Na]⁺

### 2-(3-(aminomethyl)phenyl)-N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide hydrochloride (Compound 17)

To a stirred mixture of tert-butyl 3-(7-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentylcarbamoyl)-4-chloroquinolin-2-yl)benzylcarbamate (40 mg, 0.04 mmol) in DCM (5 mL) was added HCl solution in 1,4-dioxane (4 M, 0.5 mL, 2 mmol). The mixture was stirred at rt for 3 hours and concentrated in vacuum, the residue was triturated with Et₂O to give compound 17 (36 mg, yield 95%) as a white solid. ¹H NMR (400 MHz, CD₃OD) δ (ppm) 9.95 (d, J=12.0 Hz, 1H), 8.49-8.46 (m, 1H), 8.25-8.15 (m, 4H), 8.12-7.92 (m, 3H), 7.57-7.47 (m, 3H), 7.21-6.92 (m, 5H), 4.10 (s, 2H), 3.98-3.23 (m, 15H), 3.08-2.92 (m, 3H), 2.84 (s, 3H), 2.70-2.58 (m, 3H), 1.72-0.91 (m, 8H). LCMS: (m/z) 912.4 [M+H]⁺

### Synthesis of Compound 18

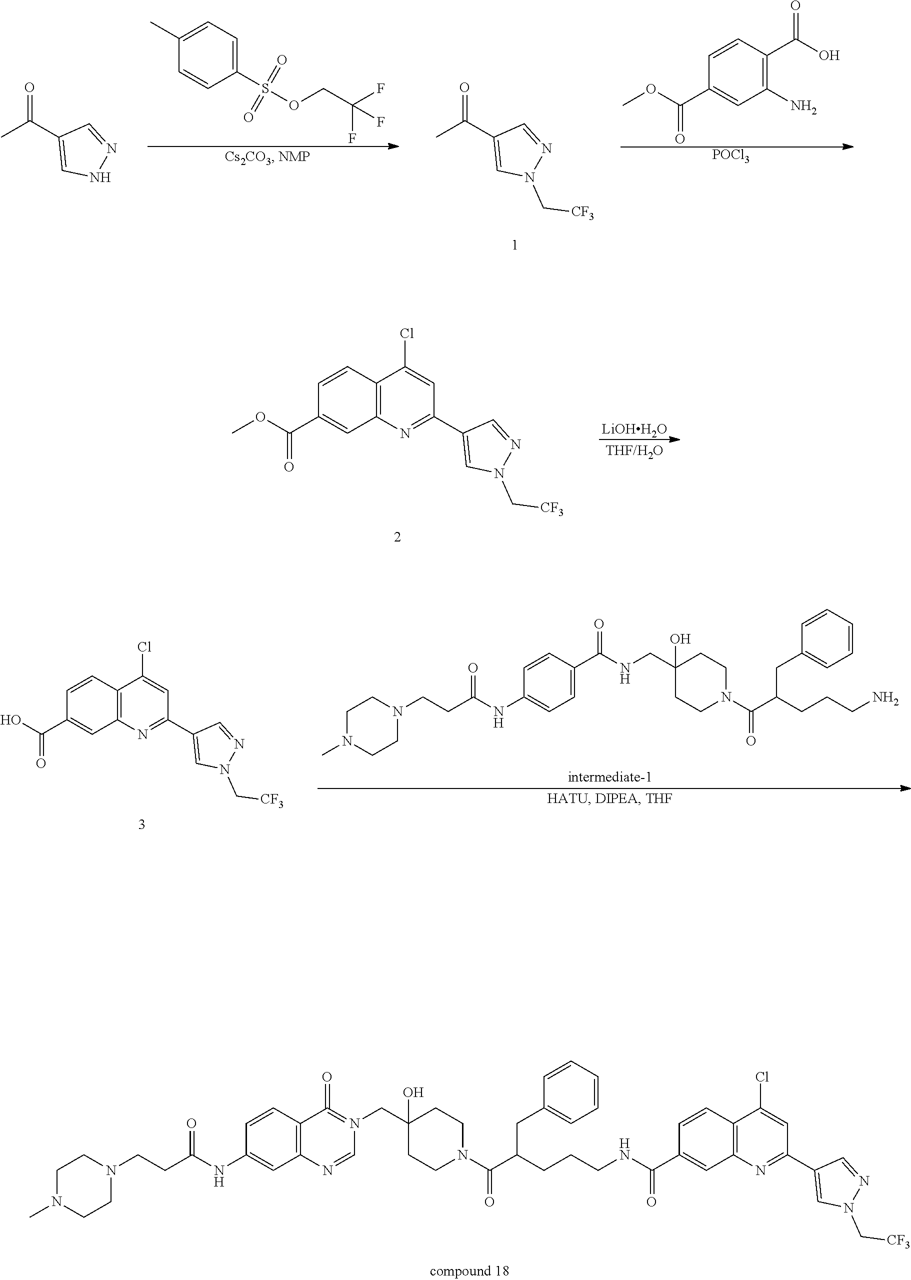

### 1-(1-(2,2,2-trifluoroethyl)-1H-pyrazol-4-yl)ethanone (Intermediate 1)

To a mixture of 1-(1H-pyrazol-4-yl)ethanone (500 mg, 4.54 mmol) and 2,2,2-trifluoroethyl 4-methylbenzenesulfonate (1270 mg, 4.99 mmol) in NMP (10 mL) was added Cs₂CO₃ (2219 mg, 6.81 mmol). The mixture was stirred at 60° C. for 18 h, after cooled down to rt, the mixture was diluted with H₂O (30 mL) and extracted with EtOAc (3×40 mL). The combined organics were washed with brine (2×20 mL), dried over anhydrous Na₂SO₄, filtered, concentrated and purified by flash column chromatograph (0-50% EtOAc in PE) to give intermediate 1 (720 mg, Yield 82.6%) as an oil. LCMS: (m/z) 193.1 [M+H]⁺,

### methyl 4-chloro-2-(1-(2,2,2-trifluoroethyl)-1H-pyrazol-4-yl)quinoline-7-carboxylate (Intermediate 2)

A mixture of intermediate 1 (720 mg, 3.75 mmol) and 2-amino-4-(methoxycarbonyl)benzoic acid (1098 mg, 5.625 mmol) in POCl₃ (10 mL) was stirred at 100° C. for 3 hour, after cooled down to rt, the mixture was concentrated in vacuum, the residue was taken into ice water (20 mL), adjusted to pH 8-9 with solid NaHCO₃ and extracted with DCM (50 mL×3). The combined organics was dried over anhydrous Na₂SO₄, filtered, concentrated and purified by flash column chromatograph (DCM/MeOH=40/1) to give intermediate 2 (105 mg, Yield 7.5%) as pale yellow solid. LCMS: (m/z) 370.1 [M+H]⁺

### 4-chloro-2-(1-(2,2,2-trifluoroethyl)-1H-pyrazol-4-yl)quinoline-7-carboxylic acid (Intermediate 3)

To a solution of methyl 4-chloro-2-(1-(2,2,2-trifluoroethyl)-1H-pyrazol-4-yl)quinoline-7-carboxylate (105 mg, 0.284 mmol) in THF (50 mL) was added a solution of LiOH·H₂O (18 mg, 0.426 mmol) in H₂O (2 mL). The mixture was stirred at rt for 16 hours and then concentrated in vacuum. The residue was taken up in water (20 mL) and acidified with 1 M HCl solution (pH 4-5), the resulting mixture was extracted with ethyl acetate (50 mL×3), the combined organic phase was dried over anhydrous Na₂SO₄ and concentrated in vacuum to leave crude intermediate 3 (95 mg, Yield 94.2%) as solid. LCMS: (m/z) 356.0 [M+H]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(1-(2,2,2-trifluoroethyl)-1H-pyrazol-4-yl)quinoline-7-carboxamide (Compound 18)

A mixture of 4-chloro-2-(1-(2,2,2-trifluoroethyl)-1H-pyrazol-4-yl)quinoline-7-carboxylic acid (95 mg, 0.267 mmol), N-((1-(5-amino-2-benzylpentanoyl)-4-hydroxypiperidin-4-yl)methyl)-4-(3-(4-methylpiperazin-1-yl)propanamido) benzamide hydrochloride (178 mg, 0.267 mmol), HATU (112 mg, 0.294 mmol) and DIPEA (103 mg, 0.801 mmol) in THF (10 mL) was stirred at rt for 2 hours. The mixture was concentrated and purified by prep-HPLC (C18 column, CH₃CN/H₂O, containing 0.05% NH₄HCO₃) to give compound 18 (32.5 mg, Yield 13%) as a white solid. ¹H NMR (400 MHz, CD₃OD) δ (ppm) 10.53 (d, J=4.8 Hz, 1H), 8.93-8.86 (m, 1H), 8.71 (s, 1H), 8.53 (d, J=10.4 Hz, 1H), 8.41 (s, 1H), 8.30 (d, J=3.2 Hz, 1H), 8.23-8.18 (m, 1H), 8.15-8.98 (m, 4H), 7.65-7.57 (m, 1H), 7.28-7.07 (m, 5H), 5.29 (q, J=8.4 Hz, 2H), 4.88 (s, 1H), 4.18-3.98 (m, 1H), 3.97-3.54 (m, 3H), 3.35-3.10 (m, 4H), 2.92-2.53 (m, 9H), 2.49-2.20 (m, 6H), 2.14 (s, 3H), 1.71-1.00 (m, 8H). LCMS: (m/z) 955.3 [M+H]⁺

### Synthesis of Compound 21

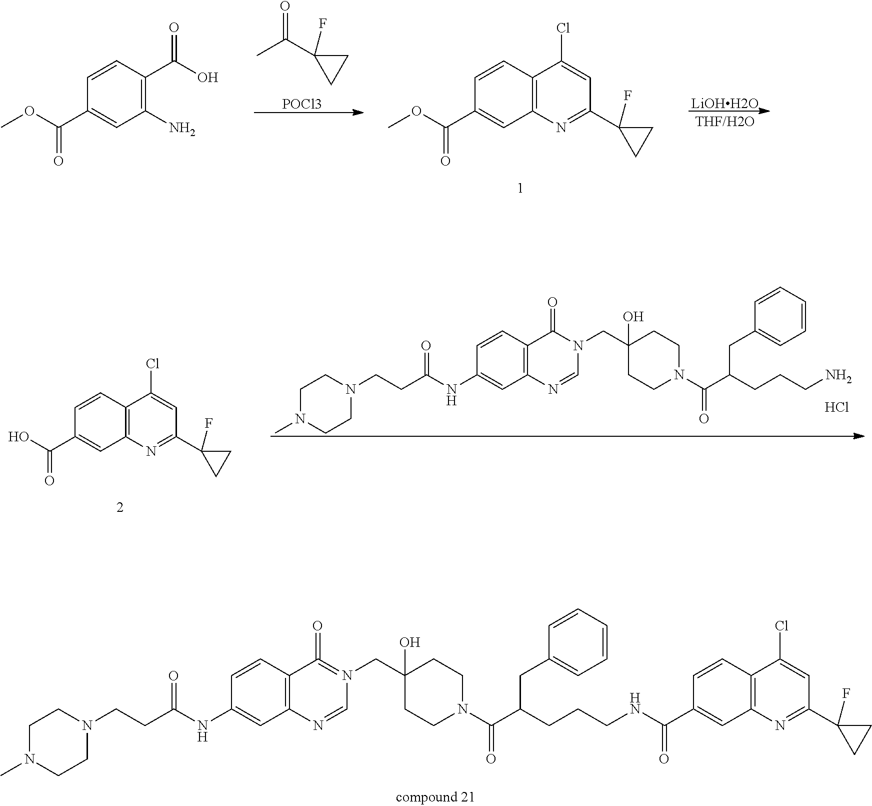

### methyl 4-chloro-2-(1-fluorocyclopropyl)quinoline-7-carboxylate (Intermediate 1)

To a mixture of 1-(1-fluorocyclopropyl) ethanone (169 mg, 1.66 mmol) in POCl₃ (6 ml) was added 2-amino-4-(methoxycarbonyl)benzoic acid (162 mg, 0.83 mmol) at rt. The mixture was heated in a microwave reactor at 100° C. for 30 min. Upon cooling to rt, the mixture was concentrated, the residue was taken into ice water (20 mL), adjusted to pH 8-9 with NaHCO₃ solution and extracted with DCM (50 mL×3). The combined organics was dried over anhydrous Na₂SO₄, filtered, concentrated and purified by flash chromatograph (DCM/MeOH=40/1) to give intermediate (12 mg, Yield 2.76%) as a solid. LCMS: (m/z) 280.1 [M+H]⁺

### 4-chloro-2-(1-fluorocyclopropyl)quinoline-7-carboxylic acid (Intermediate 2)

To a mixture of methyl 4-chloro-2-(1-fluorocyclopropyl)quinoline-7-carboxylate (12 mg, 0.043 mmol) in THF (3 ml) and H₂O (1.0 ml) was added LiOH·H₂O (2.7 mg, 0.065 mmol). The mixture was stirred at rt for 16 hours and quenched with HCl solution in 1,4-dioxane (4 M, 0.02 mL. 0.08 mmol). The mixture was concentrated to leave crude intermediate 2 (19 mg, Yield 100%) as a white solid. LCMS: (m/z) 266.1[M+H]⁺

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloro-2-(1-fluorocyclopropyl)quinoline-7-carboxamide (Compound 21)

To a stirred solution of 4-chloro-2-(1-fluorocyclopropyl)quinoline-7-carboxylic acid (19 mg, crude 0.071 mmol), N-((1-(5-amino-2-benzylpentanoyl)-4-hydroxypiperidin-4-yl)methyl)-4-(3-(4-methylpiperazin-1-yl)propanamido)benzamide hydrochloride (46 mg, 0.071 mmol) and DIEA (37 mg, 0.284 mmol) in DMF (5 mL) was added HATU (30 mg, 0.078 mmol). The mixture was stirred at rt for 1 hour, diluted with EtOAc (100 mL), washed with aqueous NaHCO₃ solution (50 mL) and brine (50 mL), dried over anhydrous Na₂SO₄, filtered, concentrated and purified by prep-HPLC (C18 column, CH₃CN/H₂O, containing 0.05% NH₄HCO₃) to give compound 21 (12.4 mg, Yield 20%) as a white solid. ¹H NMR (400 MHz, CD₃OD) δ (ppm) 10.52 (s, 1H), 8.89-8.82 (m, 1H), 8.48 (dd, J=1.2 Hz, 10.0 Hz, 1H), 8.28-8.23 (m, 1H), 8.17-7.95 (m, 5H), 7.65-7.57 (m, 1H), 7.27-7.07 (m, 5H), (m, 5H), 4.84 (s, 1H), 4.16-3.97 (m, 1H), 3.94-3.55 (m, 3H), 3.30-3.08 (m, 4H), 2.90-2.79 (m, 1H), 2.79-2.60 (m, 5H), 2.57-2.52 (m, 2H), 2.47-2.22 (m, 7H), 2.14 (s, 3H), 1.72-1.02 (m, 12H). LCMS: (m/z) 865.3[M+H]⁺

### Synthesis of Compound 23

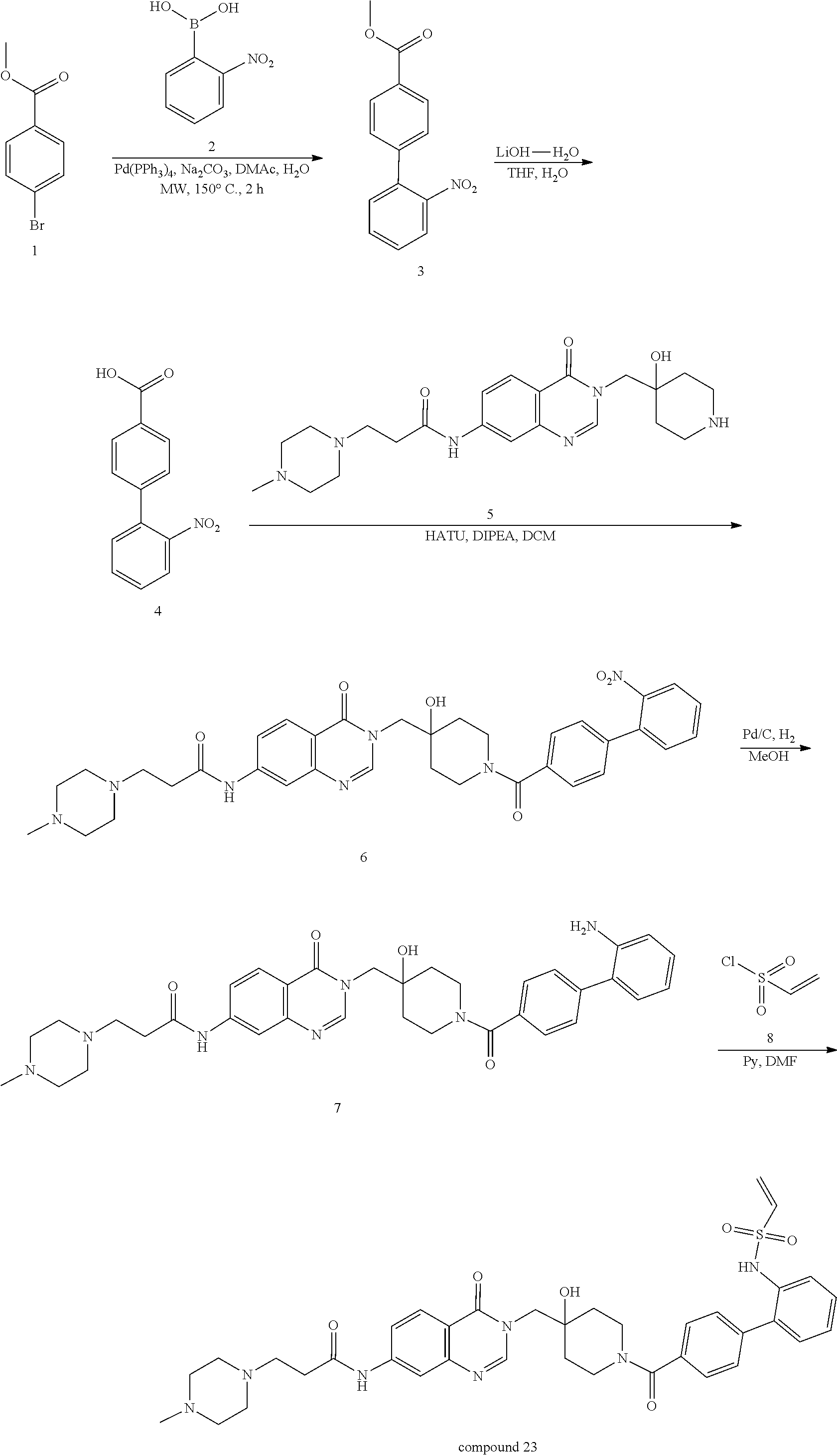

### methyl 2′-nitro-[1,1′-biphenyl]-4-carboxylate (Intermediate 3)

A mixture of methyl 4-bromobenzoate (1500 mg, 7.0 mmol), (2-nitrophenyl)boronic acid (1520 mg, 9.1 mmol), NaHCO₃ (1806 mg, 21.0 mmol) and Pd(PPh₃)₄ (404 mg, 0.35 mmol) in DMAc (20.0 mL) and H₂O (4.0 mL) was heated in a microwave reactor at 150° C. for 2 h. The mixture was diluted with H₂O (30 mL) and extracted with EtOAc (50 mL), the organic was dried over anhydrous Na₂SO₄, filtered, concentrated and purified by column chromatography on silica-gel (EA/PE=1/2) to afford intermediate 3 (1.2 g, yield 67%) as white solid. LCMS (m/z): 258.1 [M+H]⁺.

### 2′-nitro-[1,1′-biphenyl]-4-carboxylic acid (Intermediate 4)

A mixture of compound 3 (1000 mg, 3.8 mmol) and LiOH (186 mg, 7.7 mmol) in THF (15.0 mL) and H₂O (3.0 mL) was stirred at 40° C. for 16 h. The mixture was concentrated in vacuum, the residue was diluted with water (50 mL) and washed with EtOAc (50 mL), the aqueous phase was adjusted to pH 5 with concentrated HCl solution, the resulting solid was collected and dried under vacuum to afford intermediate 4 (800 mg, yield 86%) as white solid. LCMS (m/z): 244.1 [M+H]⁺.

### N-(3-((4-hydroxy-1-(2′-nitro-[1,1′-biphenyl]-4-carbonyl)piperidin-4-yl)methyl)-4-oxo-3,4-dihydroquinazolin-7-yl)-3-(4-methylpiperazin-1-yl)propanamide (Intermediate 6)

A mixture of intermediate 4 (122 mg, 0.5 mmol), HATU (209 mg, 0.55 mmol) and DIPEA (0.6 mL, 2.5 mmol) in DCM (10.0 mL) was stirred at rt for 1 h, the reaction solution was then added dropwise to the mixture of intermediate 5 (214 mg, 0.1 mmol) and DIPEA (0.1 mL, 0.5 mmol) in DCM (5.0 mL), the mixture was stirred at rt for 2 hours, diluted with DCM (50 mL), washed with water (50 mL), dried over anhydrous Na₂SO₄, filtered, concentrated and purified by column chromatography on silica-gel (MeOH/DCM=1/20) to afford intermediate 6 (250 mg, yield 76%) as yellow solid. LCMS (m/z): 654.3 [M+H]⁺.

### N-(3-((1-(2′-amino-[1,1′-biphenyl]-4-carbonyl)-4-hydroxypiperidin-4-yl)methyl)-4-oxo-3,4-dihydroquinazolin-7-yl)-3-(4-methylpiperazin-1-yl)propanamide (Intermediate 7)

A mixture of compound 6 (250 mg, 0.38 mmol) and Pd/C (120 mg) in MeOH (30.0 mL) was stirred at rt under H₂ (1 atm) for 16 h. The mixture was filtered, the filtrate was concentrated to get intermediate 7 (200 mg, yield 84%) as yellow solid. LCMS (m/z): 624.3 [M+H]⁺.

### N-(3-((4-hydroxy-1-(2′-(vinylsulfonamido)-[1,1′-biphenyl]-4-carbonyl)piperidin-4-yl)methyl)-4-oxo-3,4-dihydroquinazolin-7-yl)-3-(4-methylpiperazin-1-yl)propanamide (Compound 23)

The solution of ethenesulfonyl chloride (100 mg, 0.8 mmol) in dried DMF (1.0 mL) was added dropwise to the mixture of intermediate 7 (100 mg, 0.16 mmol) and dried pyridine (252 mg, 3.2 mmol) in dried DMF (3.0 mL) at rt, the mixture was stirred at rt for 16 h, the solvent was purged with N₂, the residue was dissolved in DMF, purified by prep-HPLC (C18 column, CH₃CN/H₂O, containing 0.05% NH₄HCO₃) to obtain compound 23 (34.3 mg, yield 30%) as white solid. ¹H NMR (DMSO-d₆, 400 MHz): δ (ppm) 10.52 (s, 1H), 9.26 (m, 1H), 8.22 (s, 1H), 8.09-8.02 (m, 3H), 7.62-7.60 (m, 1H), 7.50-7.43 (m, 4H), 7.38-7.29 (m, 4H), 6.60 (dd, J₁=16.4 Hz, J₂=10.0 Hz, 1H), 5.93 (d, J=16.8 Hz, 1H), 5.95 (d, J=10.0 Hz, 1H), 5.04 (s, 1H), 4.25-4.15 (m, 1H), 4.02 (br, 2H), 3.60-3.45 (m, 1H), 3.25-3.10 (m, 1H), 2.66-2.62 (m, 3H), 2.60-2.54 (m, 2H), 2.42-2.33 (m, 7H), 2.14 (s, 3H), 1.63-1.238 (m, 5H). LCMS (m/z): 714.1 [M+H]⁺

### Synthesis of Compound 24

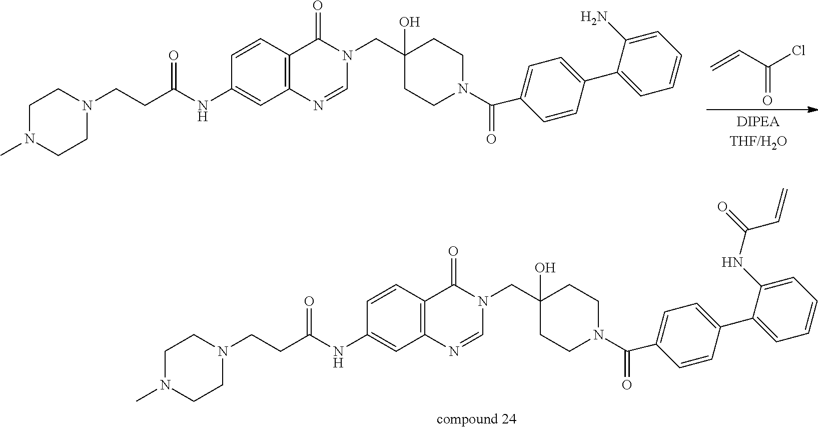

### N-(4′-(4-hydroxy-4-((7-(3-(4-methylpiperazin-1-yl)propanamido)-4-oxoquinazolin-3(4H)-yl)methyl)piperidine-1-carbonyl)biphenyl-2-yl)acrylamide (Compound 24)

To a mixture of N-(3-((1-(2′-aminobiphenylcarbonyl)-4-hydroxypiperidin-4-yl)methyl)-4-oxo-3,4-dihydroquinazolin-7-yl)-3-(4-methylpiperazin-1-yl)propanamide (40 mg, 0.064 mmol) and DIPEA (24 mg, 0.192 mmol) in THF/H₂O (6 mL/2 mL) was added acryloyl chloride (17.4 mg, 0.192 mmol) at 0° C. The mixture was stirred at rt for 3 hours, diluted with EtOAc (50 mL), washed with brine (50 mL), dried over anhydrous Na₂SO₄, concentrated and purified by prep-HPLC (C18 column, CH₃CN/H₂O, containing 0.05% NH₄HCO₃) to give compound 24 (8 mg, Yield 18.5%) as white solid. ¹H NMR (400 MHz, DMSO-d₆) δ (ppm) 10.51 (s, 1H), 9.52 (s, 1H), 8.21 (s, 1H), 8.08 (d, J=1.6 Hz, 1H), 8.01 (d, J=1.6 Hz, 1H), 7.63-7.53 (m, 2H), 7.46-7.29 (m, 7H), 6.36-6.27 (m, 1H), 6.16-6.09 (m, 1H), 5.68-5.61 (m, 1H), 5.02 (s, 1H), 4.26-3.96 (m, 4H), 3.24-3.10 (m, 1H), 2.69-2.54 (m, 4H), 2.46-2.20 (m, 7H), 2.14 (s, 3H), 1.71-1.17 (m, 5H). LCMS: (m/z) 678.3 [M+H]⁺

### Synthesis of Compound 25

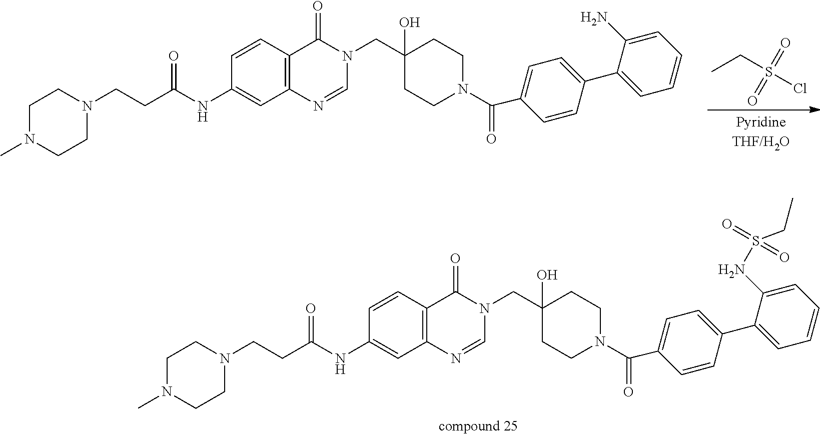

### N-(3-((1-(2′-(ethylsulfonamido)biphenylcarbonyl)-4-hydroxypiperidin-4-yl)methyl)-4-oxo-3,4-dihydroquinazolin-7-yl)-3-(4-methylpiperazin-1-yl)propenamide (Compound 25)

To a mixture of N-(3-((1-(2′-aminobiphenylcarbonyl)-4-hydroxypiperidin-4-yl)methyl)-4-oxo-3,4-dihydroquinazolin-7-yl)-3-(4-methylpiperazin-1-yl)propanamide (60 mg, 0.096 mmol) and pyridine (38 mg, 0.48 mmol) in THF/H₂O (5 mL/1 mL) was added ethanesulfonyl chloride (123 mg, 0.96 mmol) at 0° C. The mixture was stirred at rt for 3 hours. The mixture was diluted with EtOAc (50 mL), washed with brine (50 mL), dried over anhydrous Na₂SO₄, concentrated and purified by prep-HPLC (C18 column, CH₃CN/H₂O, containing 0.05% NH₄HCO₃) to afford compound 25 (20.3 mg, Yield 29.6%) as white solid. ¹H NMR (400 MHz, DMSO-d₆) δ (ppm) 10.52 (s, 1H), 8.94 (s, 1H), 8.21 (s, 1H), 8.08 (d, J=8.4 Hz, 1H), 8.01 (d, J=1.6 Hz, 1H), 7.61 (dd, J=2.0 Hz, 8.8 Hz, 1H), 7.50 (d, J=8.0 Hz, 2H), 7.45 (d, J=8.0 Hz, 2H), 7.42-7.38 (m, 2H), 7.37-7.31 (m, 2H), 5.04 (s, 1H), 4.20 (bs, 1H), 4.02 (s, 2H), 3.24-3.11 (m, 2H), 2.80 (q, J=7.2 Hz, 2H), 2.64 (t, 6 J=8.0 Hz, 3H), 2.53 (t, J=6.8 Hz, 2H), 2.48-2.23 (m, 7H), 2.15 (s, 3H), 1.73-1.29 (m, 5H), 0.97 (t, J=7.2 Hz, 3H). LCMS: (m/z) 716.7[M+H]⁺

### Synthesis of Compound 26

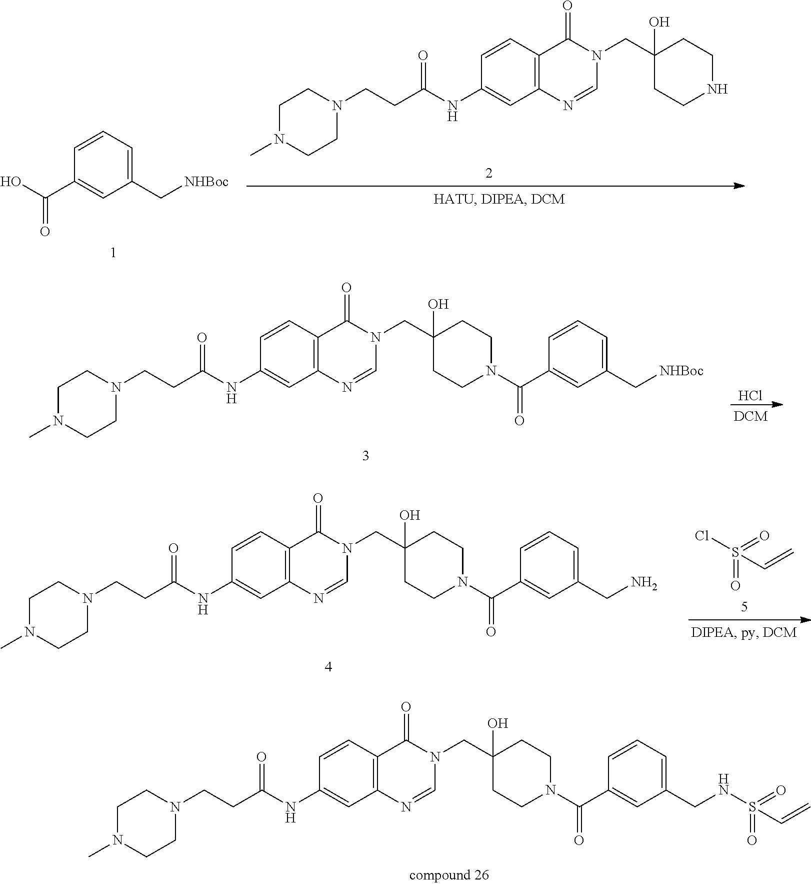

### tert-butyl (3-(4-hydroxy-4-((7-(3-(4-methylpiperazin-1-yl)propanamido)-4-oxoquinazolin-3(4H)-yl)methyl)piperidine-1-carbonyl)benzyl)carbamate (Intermediate 3)

A mixture of 3-(((tert-butoxycarbonyl)amino)methyl)benzoic acid (200 mg, 0.8 mmol), HATU (334 mg, 0.88 mmol), DIPEA (0.5 mL, 2.0 mmol) and DCM (20.0 mL) was stirred at rt for 1 h, the reaction mixture was added dropwise to the mixture of compound 2 (340 mg, 0.8 mmol) and DIPEA (0.5 mL, 2.0 mmol) in DCM (20.0 mL), the mixture was stirred at rt for 2 h., diluted with DCM (50 mL), washed with water (50 mL), dried over anhydrous Na₂SO₄, filtered, concentrated and purified by column chromatography on silica-gel (MeOH/DCM=1/20) to get intermediate 3 (400 mg, yield 75%) as yellow solid. LCMS (m/z): 662.3 [M+H]⁺.

### N-(3-((1-(3-(aminomethyl)benzoyl)-4-hydroxypiperidin-4-yl)methyl)-4-oxo-3,4-dihydroquinazolin-7-yl)-3-(4-methylpiperazin-1-yl)propanamide (Intermediate 4)

A mixture of compound 3 (400 mg, 0.6 mmol), HCl solution in 1,4-dioxane (3.0 mL) and DCM (5.0 mL) was stirred at rt for 2 hours, the mixture was concentrated in vacuum to get the crude product (300 mg) as solid. LCMS (m/z): 562.3 [M+H]⁺.

### N-(3-((4-hydroxy-1-(3-(vinylsulfonamidomethyl)benzoyl)piperidin-4-yl)methyl)-4-oxo-3,4-dihydroquinazolin-7-yl)-3-(4-methylpiperazin-1-yl)propanamide (Compound 26)

Ethylenesulfonyl chloride (220 mg, 1.75 mmol) was added dropwise to the mixture of compound 4 (200 mg, 0.35 mmol), DIPEA (225 mg, 1.75 mmol) and Py (4.0 mL) and dry DCM (2.0 mL), the mixture was stirred at rt for 2 hours, quenched with water (0.5 mL) and the solvents was blowed off with N₂, the residue was dissolved in DMSO (1 mL) and purified by prep-HPLC (C18 column, CH₃CN/H₂O, containing 0.05% NH₄HCO₃) to obtain compound 26 (15 mg, yield 6.6%) as white solid. ¹H NMR (400 MHz, DMSO-d₆): δ (ppm) 10.52 (s, 1H), 8.20 (s, 1H), 8.08-7.89 (m, 3H), 7.62-7.59 (m, 1H), 7.43-7.27 (m, 4H), 6.69 (dd, J₁=16.4 Hz, J₂=10.0 Hz, 1H), 6.03 (d, J=16.4 Hz, 1H), 5.95 (d, J=10.0 Hz, 1H), 5.02 (s, 1H), 4.27-4.00 (m, 5H), 3.20-3.09 (m, 3H), 2.67-2.62 (m, 2H), 2.54-2.50 (m, 2H), 2.42-2.17 (m, 7H), 2.13 (s, 3H), 1.59-1.29 (m, 5H). LCMS (m/z): 652.1 [M+H]⁺

### Synthesis of Compound 29

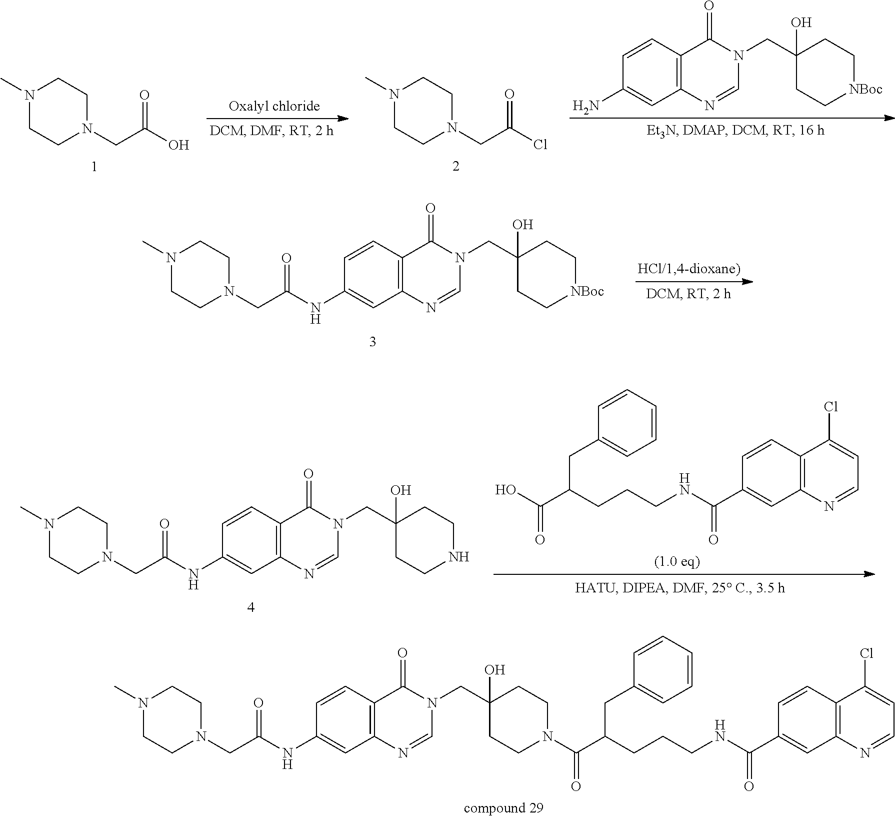

### 2-(4-methylpiperazin-1-yl)acetyl chloride (Intermediate 2)

To a solution of 2-(4-methylpiperazin-1-yl)acetic acid (474.9 mg, 3.002 mmol, 1.0 eq) in anhydrous dichloromethane (30 mL) and N,N-dimethylformamide (0.1 mL) was added oxalyl chloride (0.41 mL, 1.0 eq) while stirring in an ice-water bath. The resulting mixture was stirred at room temperature for 2 hours, and then concentrated in vacuum to leave the crude intermediate 2 (525.21 mg, 2.983 mmol, 99% yield) as brown solid. LCMS (m/z): 173.2 [M-Cl+OCH₃]⁺

### tert-butyl 4-hydroxy-4-((7-(2-(4-methylpiperazin-1-yl)acetamido)-4-oxoquinazolin-3(4H)-yl)methyl)piperidine-1-carboxylate (Intermediate 3)

To a mixture of tert-butyl 4-((7-amino-4-oxoquinazolin-3(4H)-yl)methyl)-4-hydroxypiperidine-1-carboxylate (374.3 mg, 1.0 mmol, 0.33 eq), Et₃N (1.06 mL, 2.67 eq) and DMAP (12.3 mg, 0.107 mmol, 0.03 eq) in anhydrous dichloromethane (20 mL) was added dropwise the solution of 2-(4-methylpiperazin-1-yl)acetyl chloride (525.21 mg, 2.983 mmol, 1.0 eq) in anhydrous dichloromethane (30 mL) while stirring in an ice-water bath. The resulting mixture was stirred at room temperature for another 16 hours, and then concentrated and purified by flash column chromatography (DCM/MeOH=9/1) to afford the target intermediate 3 (286 mg, 0.556 mmol, 55.6% yield) as brown solid. LC-MS (m/z): 515.3 [M+H]⁺

### N-(3-((4-hydroxypiperidin-4-yl)methyl)-4-oxo-3,4-dihydroquinazolin-7-yl)-2-(4-methylpiperazin-1-yl)acetamide (Intermediate 4)

To a solution of tert-butyl 4-hydroxy-4-((7-(2-(4-methylpiperazin-1-yl)acetamido)-4-oxoquinazolin-3(4H)-yl)methyl)piperidine-1-carboxylate (276 mg, 0.537 mmol, 1.0 eq) in dichloromethane (5 mL) was added HCl solution in 1,4-dioxane (4 M, 30 mL, 20.0 eq). The resulting mixture was stirred at room temperature for another 2 hours, and then concentrated in vacuum to leave the crude intermediate 4 (222.3 mg, 0.537 mmol, 100% yield) as white solid. LC-MS (m/z): 208.2 [M/2+H]⁺

### N-(4-benzyl-5-(4-hydroxy-4-((7-(2-(4-methylpiperazin-1-yl)acetamido)-4-oxoquinazolin-3(4H)-yl)methyl)piperidin-1-yl)-5-oxopentyl)-4-chloroquinoline-7-carboxamide (compound 29)

A mixture of 2-benzyl-5-(4-chloroquinoline-7-carboxamido)pentanoic acid (47.9 mg, 0.121 mmol, 1.0 eq), HATU (55.1 mg, 0.145 mmol, 1.2 eq) and DIPEA (31.3 mg, 0.242 mmol, 2.0 eq) in N,N-dimethylromamide (2 mL) was stirred at 25° C. for 1.5 hours, and then added dropwise to the mixture of N-(3-((4-hydroxypiperidin-4-yl)methyl)-4-oxo-3,4-dihydroquinazolin-7-yl)-2-(4-methylpiperazin-1-yl)acetamide (0.05 g, 0.121 mmol, 1.0 eq) and DIPEA (0.0469 g, 0.363 mmol, 3.0 eq) in N,N-dimethylromamide (2 mL), the resulting mixture was stirred at 25° C. for another 3.5 hours, concentrated and purified by prep-HPLC to afford the target compound 29 (11.3 mg, yield 11.79%) as white solid. ¹H NMR (400 MHz, DMSO-d₆): δ (ppm) 10.15 (brs, 1H), 8.98-8.84 (m, 2H), 8.60 (d, J=5.2 Hz, 1H), 8.32-8.01 (m, 5H), 7.86 (d, J=4.8 Hz, 1H), 7.74-7.65 (m, 1H), 7.28-7.07 (m, 5H), 4.86 (s, 1H), 4.17-3.57 (m, 6H), 3.21-3.07 (m, 5H), 2.89-2.64 (m, 5H), 2.45-2.26 (m, 3H), 2.18 (s, 3H), 1.69-1.03 (m, 10H). LC-MS (m/z): 793.3 [M+H]⁺

### Synthesis of Compound 30

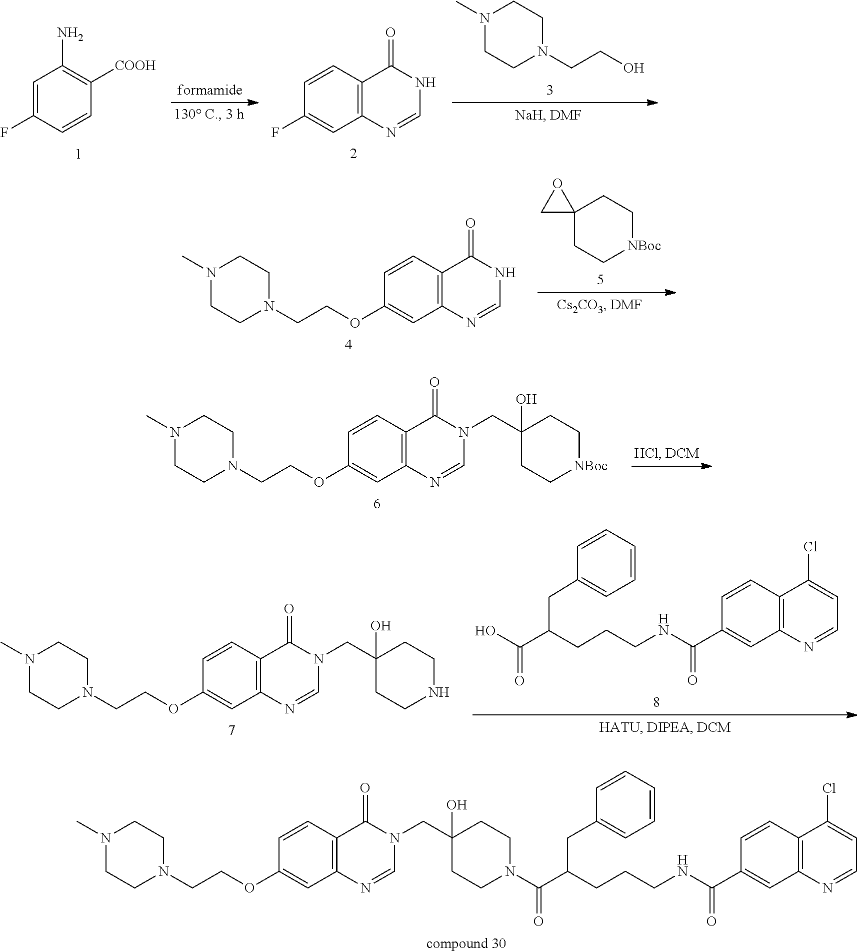

### 7-fluoroquinazolin-4(3H)-one (Intermediate 2)

The mixture of 2-amino-4-fluorobenzoic acid (1550 mg, 10 mmol) in formamide (6.0 mL) was stirred at 130° C. for 3 h. After cooled down to rt the reaction mixture was poured into water (100 mL), the resulting solid was collected and dried under vacuum to get intermediate 2 (1.3 g, yield 78%) as brown solid. LCMS (m/z): 165.1 [M+H]⁺. 7-(2-(4-methylpiperazin-1-yl)ethoxy)quinazolin-4(3H)-one (Intermediate 4) NaH (1100 mg, 27.5 mmol) was added portionwise to the mixture of intermediate 3 (1584 mg, 11.0 mmol) in DMF (20.0 mL), the mixture was stirred at rt for 1 h, and then than 2-(4-methylpiperazin-1-yl)ethan-1-ol (900 mg, 5.5 mmol) was added, the resulting mixture was stirred at 100° C. for 2 h. After cooled down to rt the reaction mixture was diluted with water (100 mL) and washed with DCM (50 mL), the aqueous phase was concentrated in vacuum, the residue was dissolved in a mixture (DCM/EA=3/1 (80 mL)), stirred at rt for 3 h and filtered to get intermediate 4 (1.2 g, yield 75%) as yellow solid. LCMS (m/z): 289.2 [M+H]⁺.

### tert-butyl 4-hydroxy-4-((7-(2-(4-methylpiperazin-1-yl)ethoxy)-4-oxoquinazolin-3(4H)-yl)methyl)piperidine-1-carboxylate (Intermediate 6)

A mixture of intermediate 4 (800 mg, 2.8 mmol), tert-butyl 1-oxa-6-azaspiro[2.5]octane-6-carboxylate (715 mg, 3.3 mmol) and Cs₂CO₃ (1369 mg, 4.2 mmol) in DMF (12.0 mL) was stirred at 80° C. for 16 h. After cooled down to rt the reaction mixture was diluted with EtOAc (100 mL), washed with water (20 mL*4), dried over anhydrous Na₂SO₄, filtered, concentrated and purified by column chromatography on silica-gel (MeOH/DCM=1/20) to obtain intermediate 6 (290 mg, yield 21%) as yellow solid. LCMS (m/z): 502.4 [M+H]⁺.

### 3-((4-hydroxypiperidin-4-yl)methyl)-7-(2-(4-methylpiperazin-1-yl)ethoxy)quinazolin-4(3H)-one (Intermediate 7)

To the mixture of intermediate 6 (90 mg, 0.18 mmol) in DCM (2.0 mL) was added HCl solution in 1,4-dioxane (4M, 1.0 mL), the mixture was stirred at rt for 2 h and concentrated in vacuum to leave the crude intermediate 7 (80 mg,) as solid. LCMS (m/z): 402.1 [M+H]⁺.

### N-(4-benzyl-5-(4-hydroxy-4-((7-(2-(4-methylpiperazin-1-yl)ethoxy)-4-oxoquinazolin-3(4H)-yl)methyl)piperidin-1-yl)-5-oxopentyl)-4-chloroquinoline-7-carboxamide (Compound 30)

A mixture of intermediate 8 (79 mg, 0.2 mmol), HATU (84 mg, 0.22 mmol) and DIPEA (1.0 mL, 0.5 mmol) in DCM (10.0 mL) was stirred at rt for 30 min, the mixture was then added dropwise to the solution of intermediate 7 (80 mg, 0.2 mmol) and DIPEA (1.0 mL, 0.5 mmol) in DCM (10.0 mL), the resulting mixture was stirred at rt for 2 h, diluted with water (20 mL) and extracted with DCM (20 mL×3), the combined organic was dried over anhydrous Na₂SO₄, filtered, concentrated and purified by prep-HPLC (C18 column, CH₃CN/H₂O, containing 0.05% NH₄HCO₃) to obtain compound 30 (38 mg, yield 24%) as white solid. ¹H NMR (400 MHz, DMSO-d₆): δ (ppm) 8.94-8.87 (m, 2H), 8.61-8.59 (m, 1H), 8.29-8.26 (m, 1H), 8.20-7.98 (m, 3H), 7.86 (d, J=4.8 Hz, 1H), 7.26-7.07 (m, 7H), 4.84 (s, 1H), 4.23-4.19 (m, 2H), 4.15-3.60 (m, 4H), 3.31-3.13 (m, 4H), 2.87-2.65 (m, 6H), 2.51-2.40 (m, 7H), 2.15 (s, 3H), 1.64-1.06 (m, 8H). LCMS (m/z): 780.2 [M+H]

### Synthesis of Compound 31

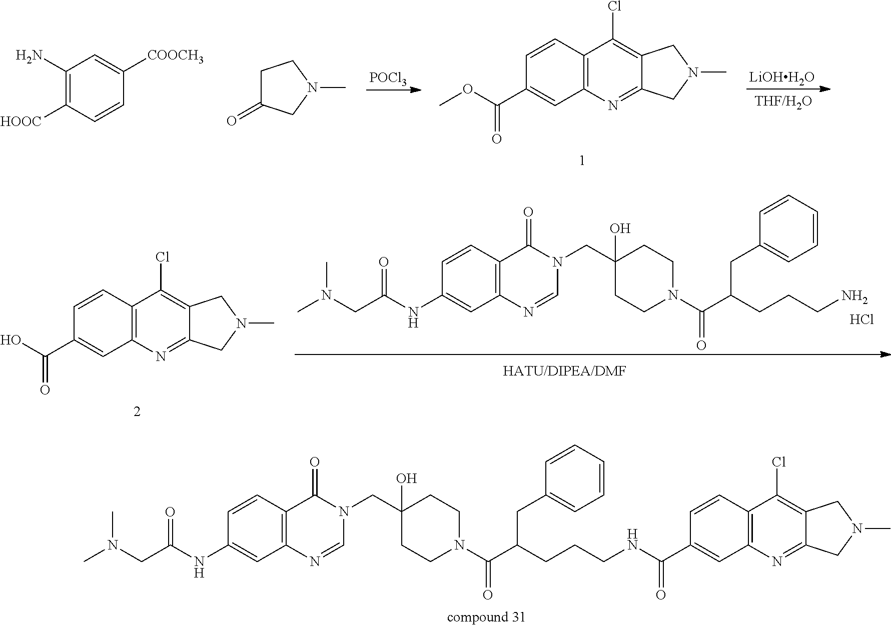

### methyl 9-chloro-2-methyl-2,3-dihydro-1H-pyrrolo[3,4-b]quinoline-6-carboxylate (Intermediate 1)

A mixture of 2-amino-4-(methoxycarbonyl)benzoic acid (390 mg, 2.0 mmol) and 1-methylpyrrolidin-3-one (990 mg, 10.0 mmol) in POCl₃ (10 mL) was stirred at 100° C. for 30 minutes under microwave irradiation. The mixture was concentrated in vacuum, the residue was diluted with DCM (100 mL), and adjusted to pH 7-8 with aqueous Na₂CO₃ solution. The organic layer was separated, the aqueous layer was extracted with DCM (50 mL×3). The combined organics was washed with brine (100 mL), dried over anhydrous Na₂SO₄, concentrated and purified by flash chromatograph (DCM/MeOH=30/1) to give intermediate 1 (62 mg, yield 10.7%) as a brown solid. LCMS: (m/z) 277.1 [M+H]⁺

### 9-chloro-2-methyl-2,3-dihydro-1H-pyrrolo[3,4-b]quinoline-6-carboxylic acid (Intermediate 2)

To a mixture of methyl 9-chloro-2-methyl-2,3-dihydro-1H-pyrrolo[3,4-b]quinoline-6-carboxylate (62 mg, 0.224 mmol) in THF/H₂O (10 mL/2 mL) was added LiOH·H₂O (14 mg, 0.336 mmol). The mixture was stirred at rt for 16 hours and then HCl solution in 1,4-Dioxane (4 M, 0.1 mL, 0.4 mmol) was added, the mixture was concentrated and dried under vacuum to leave crude intermediate 2 (70 mg, yield 100%) as yellow solid. LCMS: (m/z) 263.0 [M+H]⁺

### N-(4-benzyl-5-(4-((7-(2-(dimethylamino)acetamido)-4-oxoquinazolin-3(4H)-yl)methyl)-4-hydroxypiperidin-1-yl)-5-oxopentyl)-9-chloro-2-methyl-2,3-dihydro-1H-pyrrolo[3,4-b]quinoline-6-carboxamide (Compound 31)

To a stirred solution of 9-chloro-2-methyl-2,3-dihydro-1H-pyrrolo[3,4-b]quinoline-6-carboxylic acid (32 mg, crude 0.1 mmol), N-(3-((1-(5-amino-2-benzylpentanoyl)-4-hydroxypiperidin-4-yl)methyl)-4-oxo-3,4-dihydroquinazolin-7-yl)-2-(dimethylamino)acetamide hydrochloride (58 mg, 0.1 mmol) and DIEA (52 mg, 0.4 mmol) in DMF (5 mL) was added HATU (421 mg, 0.11 mmol). The mixture was stirred at rt for 1 hour and then concentrated, the residue was purified by prep-HPLC (C18 column, CH₃CN/H₂O, containing 0.05% NH₄HCO₃) to afford compound 31 (34.5 mg, Yield 43%) as white solid. ¹H NMR (400 MHz, DMSO-d₆) δ (ppm) 10.20 (d, 4.8 Hz, 1H), 8.85-8.77 (m, 1H), 8.52 (dd, J=1.2 Hz, 11.6 Hz, 1H), 8.22-8.16 (m, 1H), 8.15-8.00 (m, 4H), 7.79-7.00 (m, 1H), 7.27-7.07 (m, 5H), 4.83 (d, J=2.4 Hz, 1H), 4.16-3.96 (m, 5H), 3.95-3.76 (m, 2H), 3.67-3.56 (m, 2H), 3.30-3.23 (m, 2H), 3.15 (s, 3H), 2.90-2.60 (m, 3H), 2.59-2.53 (m, 3H), 2.30 (s, 6H), 1.69-0.99 (m, 8H). LCMS: (m/z) 793.3 [M+H]⁺

### Synthesis of Compound 32

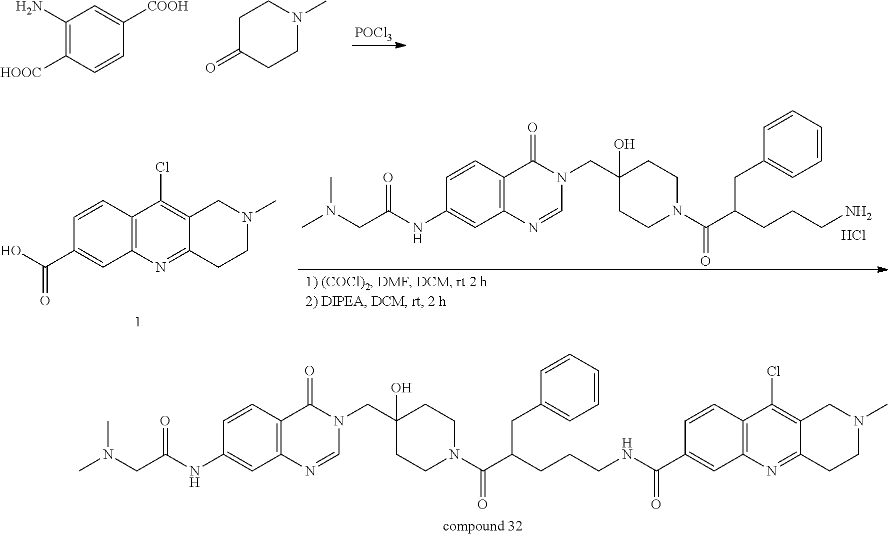

### 10-chloro-2-methyl-1,2,3,4-tetrahydrobenzo[b][1,6]naphthyridine-7-carboxylic acid (Intermediate 1)

A mixture of 2-aminoterephthalic acid (181 mg, 1.0 mmol) and 1-methylpiperidin-4-one (226 mg, 2.0 mmol) in POCl₃ (8 mL) was stirred at 100° C. for for 30 minutes under microwave irradiation. The mixture was concentrated in vacuum, the residue was diluted with H₂O (10 mL) and purified by prep-HPLC (C18 column, CH₃CN/H₂O, containing 0.05% NH₄HCO₃) to give intermediate 1 (47.8 mg, Yield 15.7%) as a white solid. LCMS: (m/z) 277.1 [M+H]⁺

### N-(4-benzyl-5-(4-((7-(2-(dimethylamino)acetamido)-4-oxoquinazolin-3(4H)-yl)methyl)-4-hydroxypiperidin-1-yl)-5-oxopentyl)-10-chloro-2-methyl-1,2,3,4-tetrahydrobenzo[b][1,6]naphthyridine-7-carboxamide (Compound 32)

To a stirred solution of 10-chloro-2-methyl-1,2,3,4-tetrahydrobenzo[b][1,6]naphthyridine-7-carboxylic acid (47.8 mg, 0.173 mmol) in dried DCM (10 mL) was added dried DMF (0.05 mL), followed by addition of (COCl)₂ (33 mg, 0.26 mmol) at 0° C. The mixture was stirred at rt for 3 hours and concentrated in vacuum, the residue was diluted with DCM (10 mL) and added dropwise to the mixture of N-(3-((1-(5-amino-2-benzylpentanoyl)-4-hydroxypiperidin-4-yl)methyl)-4-oxo-3,4-dihydroquinazolin-7-yl)-2-(dimethylamino)acetamide hydrochloride (101 mg, 0.173 mmol) and DIEA (89 mg, 0.692 mmol) in DCM (10 mL). The resulting mixture was stirred at rt for 2 hours, concentrated and purified by prep-HPLC (C18 column, CH₃CN/H₂O, containing 0.05% NH₄HCO₃) to afford compound 32 (20.7 mg, Yield 15%) as white solid. ¹H NMR (400 MHz, DMSO-d₆) δ (ppm) 10.20 (d, J=5.2 Hz, 1H), 8.84-8.73 (m, 1H), 8.48 (d, J=11.2 Hz, 1H), 8.23-7.98 (m, 5H), 7.79-7.70 (m, 1H), 7.30-7.05 (m, 5H), 4.83 (s, 1H), 4.17-3.95 (m, 1H), 3.95-3.54 (m, 5H), 3.29-3.22 (m, 2H), 3.20-2.60 (m, 11H), 2.47 (s, 3H), 2.29 (s, 6H), 1.71-0.99 (m, 8H). LCMS: (m/z) 807.3[M+H]⁺

### Synthesis of Compound 33

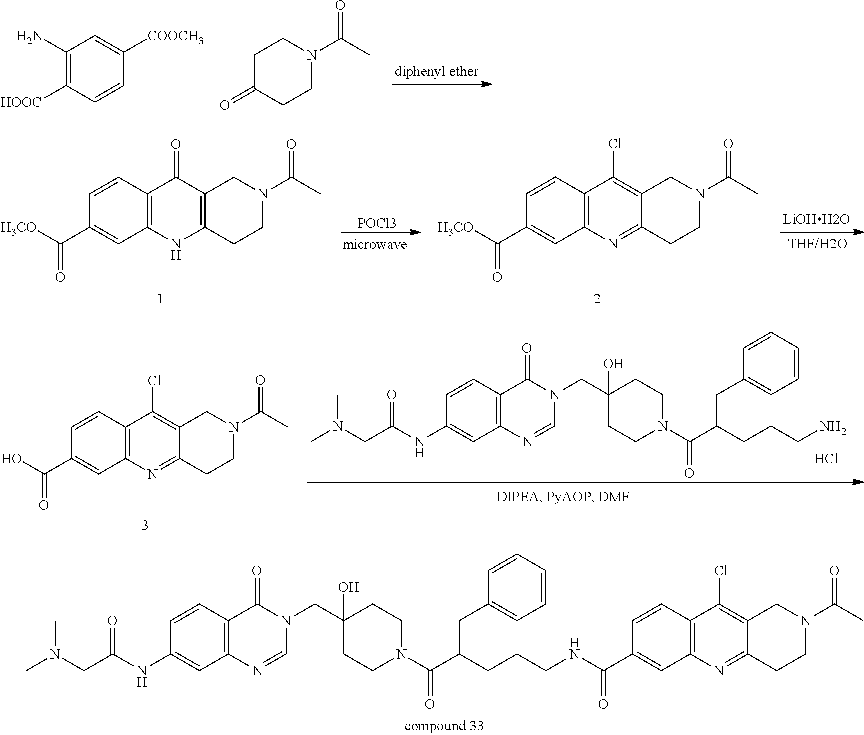

### methyl 2-acetyl-10-oxo-1,2,3,4,5,10-hexahydrobenzo[b][1,6]naphthyridine-7-carboxylate (Intermediate 1)

A mixture of 2-amino-4-(methoxycarbonyl)benzoic acid (586 mg, 3.0 mmol) and 1-acetylpiperidin-4-one (847 mg, 6.0 mmol) in diphenyl ether (30 mL) was stirred at 200° C. for 4 hour. After cooled down to rt PE (200 mL) was added, the mixture was stirred at rt for 30 minutes and filtered, the cake was purified by flash chromatograph (DCM/MeOH=30/1) to give intermediate 1 (300 mg, yield 33%) as a brown solid. LCMS: (m/z) 301.1[M+H]⁺

### methyl 2-acetyl-10-chloro-1,2,3,4-tetrahydrobenzo[b][1,6]naphthyridine-7-carboxylate (Intermediate 2)

A mixture of 1-acetylpiperidin-4-one (300 mg, 1.0 mmol) in POCl₃ (10 mL) was stirred at 80° C. for 20 minutes under microwave irradiation After cooled down to rt the mixture was concentrated in vacuum, the residue was diluted with H₂O (20 mL), adjusted to pH 5-6 and extracted with DCM (3×50 mL), the combined organics were washed with brine (100 ml), dried over anhydrous Na₂SO₄, concentrated and purified by flash column chromatograph (DCM/MeOH=30/1) to give intermediate 2 (125 mg, Yield 39%) as a brown solid. LCMS: (m/z) 319.1[M+H]⁺

### 2-acetyl-10-chloro-1,2,3,4-tetrahydrobenzo[b][1,6]naphthyridine-7-carboxylic acid (Intermediate 3)

To a mixture of methyl 2-acetyl-10-chloro-1,2,3,4-tetrahydrobenzo[b][1,6]naphthyridine-7-carboxylate (125 mg, 0.393 mmol) in THF/H₂O (10 mL/2 mL) was added LiOH·H₂O (25 mg, 0.59 mmol). The mixture was stirred at rt for 16 hours and then HCl solution in 1,4-Dioxane (4 M, 0.2 mL, 0.8 mmol) was added, the mixture was concentrated and dried under vacuum to leave crude intermediate 3 (100 mg, yield 84%) as yellow solid. LCMS: (m/z) 305.1[M+H]⁺

### 2-acetyl-N-(4-benzyl-5-(4-((7-(2-(dimethylamino)acetamido)-4-oxoquinazolin-3(4H)-yl)methyl)-4-hydroxypiperidin-1-yl)-5-oxopentyl)-10-chloro-1,2,3,4-tetrahydrobenzo[b][1,6]naphthyridine-7-carboxamide (Compound 33)

To a stirred mixture of 2-acetyl-10-chloro-1,2,3,4-tetrahydrobenzo[b][1,6]naphthyridine-7-carboxylic acid (80 mg, 0.263 mmol) and N-(3-((1-(5-amino-2-benzylpentanoyl)-4-hydroxypiperidin-4-yl)methyl)-4-oxo-3,4-dihydroquinazolin-7-yl)-2-(dimethylamino)acetamide hydrochloride (154 mg, 0.263 mmol) in DMF (5 mL) was added DIPEA (93 mg, 0.72 mmol) and PyAOP (137 mg, 0.289 mmol). The mixture was stirred at rt for 1 hour and then purified by prep-HPLC (C18 column, CH₃CN/H₂O, containing 0.05% NH₄HCO₃) to afford compound 33 (24.4 mg, Yield 12.1%) as white solid. ¹H NMR (400 MHz, DMSO-d₆, 60° C.) δ (ppm) 10.00 (s, 1H), 8.61 (s, 1H), 8.48 (s, 1H), 8.21 (d, J=8.8 Hz, 1H), 8.16-7.97 (m, 4H), 7.73 (d, J=7.6 Hz, 1H), 7.27-7.05 (m, 5H), 4.88 (s, 2H), 4.70 (s, 1H), 4.15-3.54 (m, 6H), 3.37-3.18 (m, 7H), 3.06-2.60 (m, 5H), 2.31 (s, 6H), 2.16 (s, 2H), 1.76-1.06 (m, 8H). LCMS: (m/z) 835.3 [M+H]⁺

### Synthesis of Compound 34

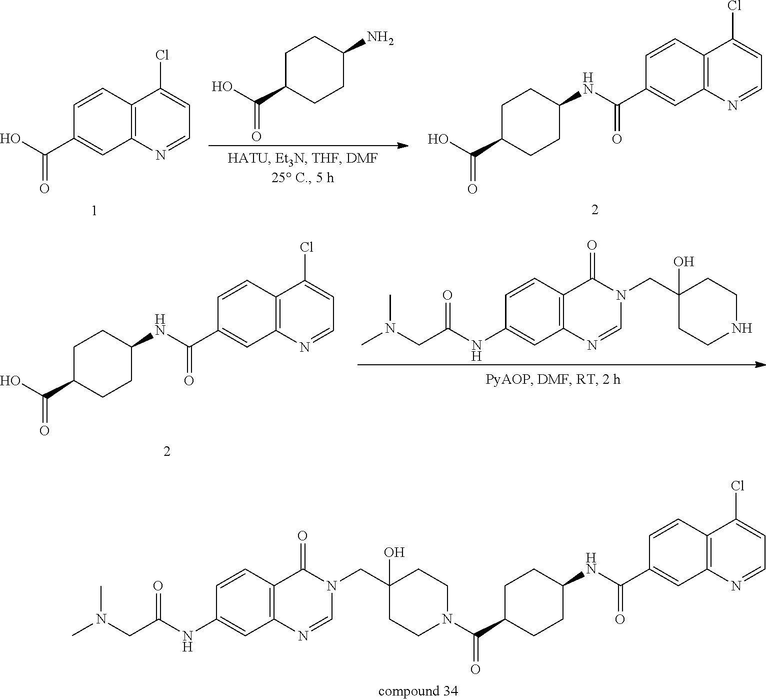

### (1s,4s)-4-(4-chloroquinoline-7-carboxamido)cyclohexanecarboxylic acid (Intermediate 2)

A mixture of 4-chloroquinoline-7-carboxylic acid (0.1235 g, 0.592 mmol, 1.0 eq), HATU (0.2399 g, 0.631 mmol, 1.05 eq) and Et₃N (0.1236 g, 1.221 mmol, 2.0 eq) in tetrahydrofuran (15 mL) and N,N-dimethylformamide (0.1 mL) was stirred at 25° C. for an hour, and then (1s,4s)-4-aminocyclohexanecarboxylic acid (0.1269 g, 0.887 mmol, 1.5 eq) was added. The resulting mixture was stirred at 25° C. for another 5 hours. Water (10 mL) was added into the mixture and layers separated. The aqueous phase was extracted with ethyl acetate (150 mL×3). The combined organic phase was washed with saturated NaCl solution (100 mL), dried over anhydrous Na₂SO₄, filtered, concentrated and purified by prep-HPLC to afford the target intermediate 2 (43 mg, 0.129 mmol, 21.87%) as white solid. LCMS (m/z): 333.1 [M+H]⁺

### 4-chloro-N-((1s,4s)-4-(4-((7-(2-(dimethylamino)acetamido)-4-oxoquinazolin-3(4H)-yl)methyl)-4-hydroxypiperidine-1-carbonyl)cyclohexyl)quinoline-7-carboxamide (Compound 34)

A mixture of intermediate 2 (43 mg, 0.129 mmol, 1.0 eq) and 2-(dimethylamino)-N-(3-((4-hydroxypiperidin-4-yl)methyl)-4-oxo-3,4-dihydroquinazolin-7-yl)acetamide (55.7 mg, 0.155 mmol, 1.2 eq) in N,N-dimethylformamide (8 mL) was stirred at room temperature for an hour. And then PyAOP (73.5 mg, 0.141 mmol, 1.1 eq) was added. The resulting mixture was stirred at room temperature for another 2 hours, diluted with Ethyl acetate (50 mL), washed with saturated Na₂CO₃ solution (100 mL) and brine (50 mL), dried over anhydrous Na₂SO₄, filtered, concentrated and purified by prep-HPLC to afford the target compound 34 (22.4 mg, 25.79%) as white solid. ¹H NMR (400 MHz, DMSO-d₆, 60° C.) δ (ppm) 10.23 (s, 1H), 8.94 (d, J=4.8 Hz, 1H), 8.70-8.62 (m, 2H), 8.28-8.07 (m, 5H), 7.86 (d, J=4.8 Hz, 1H), 7.76 (dd, J=7.6 Hz, 1.2 Hz, 1H), 5.009 (s, 1H), 4.07-3.93 (m, 4H), 3.73-3.68 (m, 1H), 3.168 (s, 2H), 3.02-2.95 (m, 2H), 2.74 (br, 1H), 2.306 (s, 6H), 1.91-1.65 (m, 4H), 1.65-1.53 (m, 2H), 1.53-1.43 (m, 6H). LC-MS (m/z): 674.3 [M+H]⁺

### Synthesis of Compound 35

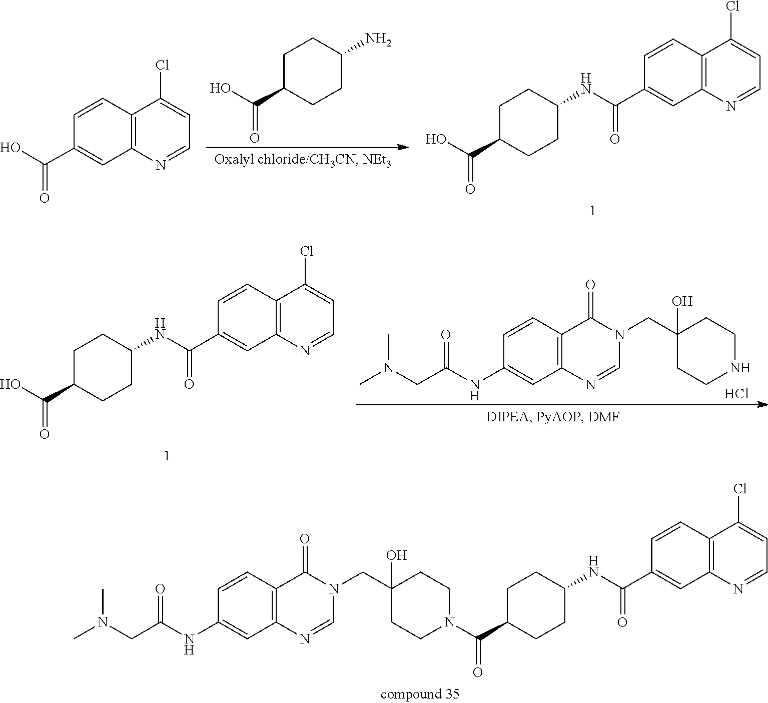

### (1R,4R)-4-(4-chloroquinoline-7-carboxamido)cyclohexanecarboxylic acid (Intermediate 1)

To a mixture of 4-chloroquinoline-7-carboxylic acid (104 mg, 0.5 mmol) in dry CH₃CN (10 mL) was added Oxalyl chloride (70 mg, 0.55 mmol) at rt. The mixture was stirred at rt for 3 hours and then added dropwise to a mixture of trans-4-aminocyclohexanecarboxylic acid (143 mg, 1.0 mmol) and TEA (202 mg, 2.0 mmol) in dry THF (10 mL). The mixture was stirred at rt for 4 hours and concentrated in vacuum, the residue was diluted with DCM (100 mL), washed with brine (100 mL), dried over anhydrous Na₂SO₄, filtered, concentrated and purified by prep-HPLC (C18 column, CH₃CN/H₂O, containing 0.05% NH₄HCO₃) to give intermediate 1 (60 mg, Yield 36%) as a white solid. LCMS: (m/z) 333.1[M+H]⁺,

### 4-chloro-N-((1R,4R)-4-(4-((7-(2-(dimethylamino)acetamido)-4-oxoquinazolin-3(4H)-yl)methyl)-4-hydroxypiperidine-1-carbonyl)cyclohexyl)quinoline-7-carboxamide (Compound 35)

To a stirred mixture of (1R,4R)-4-(4-chloroquinoline-7-carboxamido)cyclohexanecarboxylic acid (80 mg, 0.24 mmol) and 2-(dimethylamino)-N-(3-((4-hydroxypiperidin-4-yl)methyl)-4-oxo-3,4-dihydroquinazolin-7-yl)acetamide hydrochloride (95 mg, 0.24 mmol) in DMF (5 mL) was added DIPEA (93 mg, 0.72 mmol) and PyAOP (138 mg, 0.264 mmol). The mixture was stirred at rt for 1 hour and then purified by prep-HPLC (C18 column, CH₃CN/H₂O, containing 0.05% NH₄HCO₃) to afford the target compound 35 (27.2 mg, Yield 16.6%) as white solid. ¹H NMR (400 MHz, DMSO-d₆) δ (ppm) 10.19 (s, 1H), 8.93 (d, J=4.8 Hz, 1H), 8.70 (d, J=7.6 Hz, 1H), 8.61 (d, J=1.2 Hz, 1H), 8.27 (d, J=8.4 Hz, 1H), 8.22 (s, 1H), 8.17 (dd, J=1.6 Hz, 8.8 Hz, 1H), 8.12-8.05 (m, 2H), 7.86 (d, J=4.8 Hz, 1H), 7.76 (dd, J=2.0 Hz, 8.8 Hz, 1H), 4.98 (s, 1H), 4.13-4.02 (m, 2H), 3.99-3.88 (m, 1H), 3.86-3.68 (m, 2H), 3.14 (s, 2H), 3.02-2.91 (m, 1H), 2.64-2.53 (m, 1H), 2.29 (s, 6H), 1.94 (s, 2H), 1.73 (s, 2H), 1.61-1.37 (m, 8H). LCMS: (m/z) 674.3 [M+H]⁺

### Synthesis of Compound 36

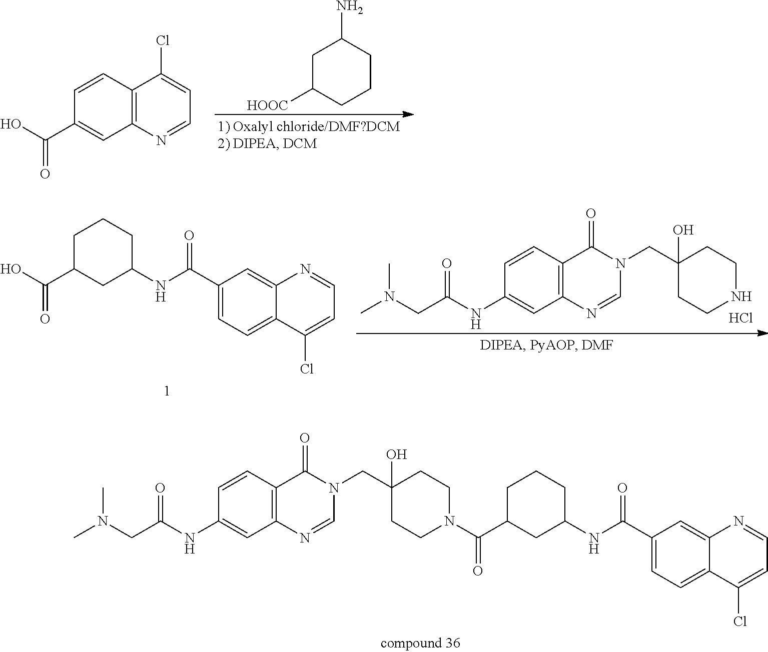

### 3-(4-chloroquinoline-7-carboxamido)cyclohexanecarboxylic acid (Intermediate 1)

To a stirred solution of 4-chloroquinoline-7-carboxylic acid (207 mg, 1.0 mmol) in dry DCM (15 mL) was added dry DMF (0.05 mL), followed by addition of (COCl)₂ (190 mg, 1.5 mmol) at 0° C. The mixture was stirred at rt for 3 hours, and concentrated in vacuum, the residue was diluted with dry DCM (15 mL) and added dropwise to the mixture of 3-aminocyclohexanecarboxylic acid (143 mg, 1.0 mmol) and DIEA (517 mg, 4.0 mmol) in dry DCM (15 mL). The resulting mixture was stirred at rt for 16 hours, quenched with MeOH (5 mL), concentrated and purified by prep-HPLC (C18 column, CH₃CN/H₂O, containing 0.05% NH₄HCO₃) to give intermediate 1 (90 mg, Yield 27%) as white solid. LCMS: (m/z) 333.1[M+H]⁺

### 4-chloro-N-(3-(4-((7-(2-(dimethylamino)acetamido)-4-oxoquinazolin-3(4H)-yl)methyl)-4-hydroxypiperidine-1-carbonyl)cyclohexyl)quinoline-7-carboxamide (Compound 36)

To a stirred mixture of 3-(4-chloroquinoline-7-carboxamido)cyclohexanecarboxylic acid (90 mg, 0.27 mmol) and 2-(dimethylamino)-N-(3-((4-hydroxypiperidin-4-yl)methyl)-4-oxo-3,4-dihydroquinazolin-7-yl)acetamide hydrochloride (107 mg, 0.27 mmol) in DMF (5 mL) was added DIPEA (105 mg, 0.81 mmol) and PyAOP (155 mg, 0.297 mmol). The mixture was stirred at rt for 1 hour, and then purified directly by prep-HPLC (C18 column, CH₃CN/H₂O, containing 0.050% NH₄HCO₃) to afford the target compound 36 (23.9 mg, Yield 13%) as white solid. ¹H NMR (400 MHz, DMSO-d₆) δ (ppm) 10.20 (d, J=3.2 Hz, 1H), 8.93 (t, J=4.8 Hz, 1H), 8.72 (t, J=7.6 Hz, 1H), 8.62 (d, J=8.8 Hz, 1H), 8.27 (t, J=8.4 Hz, 1H), 8.23-8.14 (m, 3H), 8.12-8.03 (m, 2H), 7.86 (t, J=4.8 Hz, 1H), 7.75 (t, J=7.6 Hz, 1H), 4.98 (s, 1H), 4.13-3.89 (m, 4H), 3.83-3.72 (m, 1H), 3.32-3.27 (m, 1H), 3.14 (s, 2H), 3.02-2.90 (m, 1H), 2.88-2.77 (m, 1H), 2.29 (s, 6H), 1.94-1.21 (m, 12H). LCMS: (m/z) 674.3[M+H]⁺

### Synthesis of Compound 37

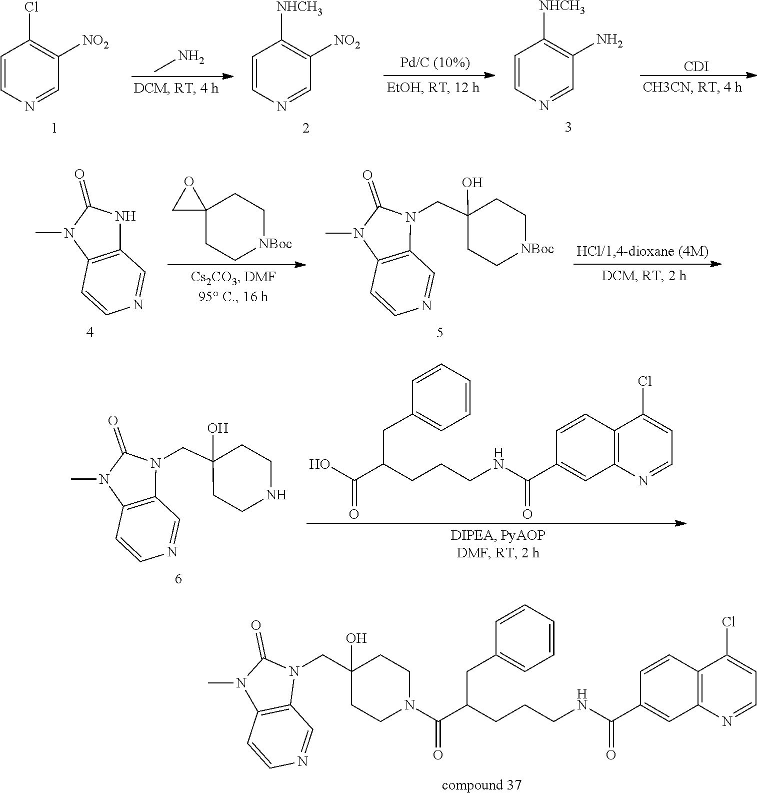

### N-methyl-3-nitropyridin-4-amine (Intermediate 2)

A mixture of 4-chloro-3-nitropyridine (2 g, 12.73 mmol, 1.0 eq), methylamine (12.7 mL, 2 M in THF, 2.0 eq) in DCM (20 mL) was stirred at room temperature for 4 hours. Water (50 mL) was added into the reaction mixture, the resulting suspension was filtered, the cake was dried under vacuum to afford the target intermediate 2 (1.74 g, 11.36 mmol, 89.31% yield) as solid. LCMS (m/z): 154.1

### N4-methylpyridine-3,4-diamine (Intermediate 3)

A mixture of intermediate 2 (1 g, 6.7 mmol, 1.0 eq) and Pd/C (10%, 0.21 g, 0.03 eq) in EtOH (10 mL) was stirred at room temperature under H₂ (1 atm) for 12 hours. The mixture was filtered through celite, the filtrate was concentrated under vacuum to afford the target intermediate 3 (0.72 g, 5.9 mmol, 88.06% yield) as solid. LCMS (m/z): 124.1

### 1-methyl-1H-imidazo[4,5-c]pyridin-2(3H)-one (Intermediate 4)

A mixture of intermediate 3 (0.4 g, 3.2 mmol, 1.0 eq) and CDI (0.53 g, 3.36 mmol, 1.05 eq) in CH₃CN (10 mL) was stirred at room temperature for 4 hours. The mixture was filtered, the solid collected was washed with CH₃CN to afford the target intermediate 4 (0.28 g, 1.9 mmol, 59.4% yield) as white powder. LCMS (m/z): 150.1

### tert-butyl 4-hydroxy-4-((1-methyl-2-oxo-1H-imidazo[4,5-c]pyridin-3(2H)-yl)methyl)piperidine-1-carboxylate (Intermediate 5)

A mixture of intermediate 4 (80 mg, 0.54 mmol, 1.0 eq), tert-butyl 1-oxa-6-azaspiro[2.5]octane-6-carboxylate (170 mg, 0.8 mmol, 1.5 eq) and Cs₂CO₃ (300 mg, 0.8 mmol, 1.5 eq) in DMF (4 mL) was stirred at 95° C. for 16 hours. The mixture was concentrated and purified by prep-HPLC to afford intermediate 5 (169 mg, 0.468 mmol, 86.69%) as white solid. LCMS (m/z): 363.2

### 3-((4-hydroxypiperidin-4-yl)methyl)-1-methyl-1H-imidazo[4,5-c]pyridin-2(3H)-one (Intermediate 6)

To a mixture of intermediate 5 (208 mg, 0.574 mmol, 1.0 eq) in DCM (10 mL) was added HCl solution in 1,4-dioxane (4 M, 3.0 mL, 20.0 eq), the mixture was stirred at room temperature for 2 hours. The solvent was then removed under vacuum to afford intermediate 6 (150 mg, 0.573 mmol, 99%) as pale yellow solid. LC-MS (m/z): 263.2 [M+H]⁺

### N-(4-benzyl-5-(4-hydroxy-4-((1-methyl-2-oxo-1H-imidazo[4,5-c]pyridin-3(2H)-yl)methyl)piperidin-1-yl)-5-oxopentyl)-4-chloroquinoline-7-carboxamide (Compound 37)

A mixture of intermediate 6 (54.5 mg, 0.208 mmol, 1.0 eq), 2-benzyl-5-(4-chloroquinoline-7-carboxamido)pentanoic acid (66.3 mg, 0.167 mmol, 1.2 eq) and DIPEA (133.7 mg, 1.035 mmol, 5.0 eq) in DMF (5 mL) was stirred at room temperature. After 10 minutes, PyAOP (99.4 mg, 0.191 mmol, 1.1 eq) was added into the reaction mixture. The resulting mixture was stirred at room temperature for 2 hours. Ethyl acetate (50 mL) was added into the reaction mixture. Saturated Na₂CO₃ solution (20 mL) was added into the mixture and layers separated. The organic phase was washed with saturated NaCl solution (50 mL), dried over anhydrous Na₂SO₄, filtered, concentrated and purified by prep-HPLC to afford the target compound 37 (16.3 mg, 12.24% yield) as white solid. ¹H NMR (400 MHz, DMSO-d₆) δ (ppm), 8.92 (d, J=4.8 Hz, 1H), 8.91-8.85 (m, 1H), 8.60 (d, J=4.8 Hz, 1H), 8.45-8.13 (m, 4H), 7.80-7.82 (m, 1H), 7.27-7.01 (m, 6H), 4.76 (d, J=5.2 Hz, 1H), 4.22-4.01 (m, 1H), 3.77-3.50 (m, 4H), 3.33-3.23 (m, 4H), 3.21-3.07 (m, 2H), 2.86-2.63 (m, 3H), 1.72-0.99 (m, 8H). LC-MS (m/z): 641.3 [M+H]⁺

### Synthesis of Compound 38

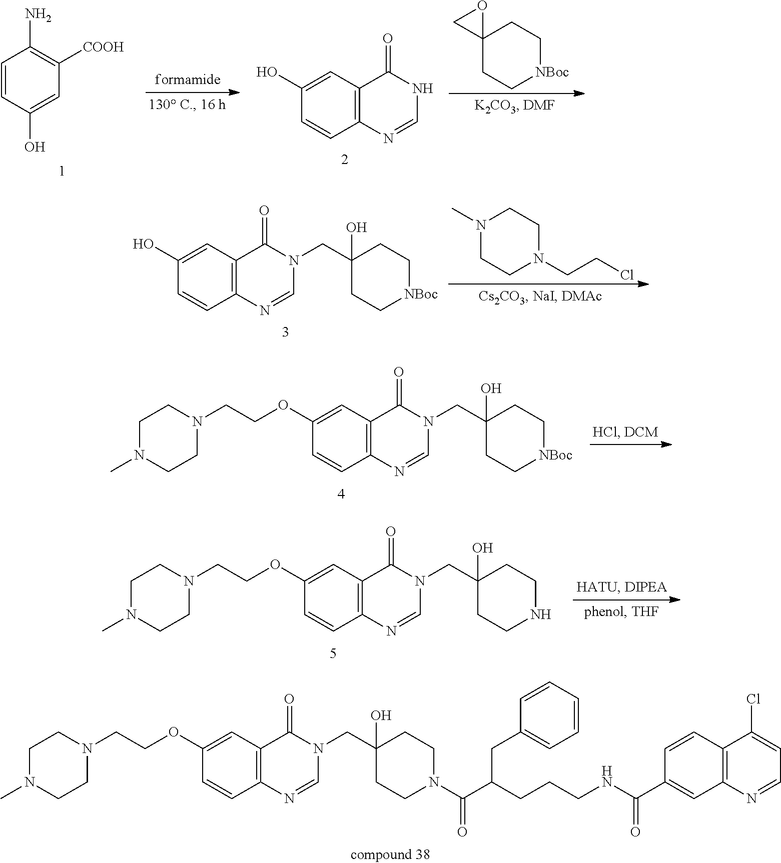

### 6-hydroxyquinazolin-4(3H)-one (Intermediate 2)

A mixture of 2-amino-5-hydroxybenzoic acid (3000 mg, 19.6 mmol) and formamide (15.0 mL) was stirred at 130° C. for 16 h. After cooled down to rt the mixture was poured into water (100 mL), the cake was collected and washed with MeOH (20 mL) to afford intermediate 2 (2.0 g, yield 62%) as brown solid. LCMS (m/z): 163.1 [M+H]⁺.

### tert-butyl 4-hydroxy-4-((6-hydroxy-4-oxoquinazolin-3(4H)-yl)methyl)piperidine-1-carboxylate (Intermediate 3)

A mixture of intermediate 2 (600 mg, 3.7 mmol), tert-butyl 1-oxa-6-azaspiro[2.5]octane-6-carboxylate (606 mg, 2.8 mmol) and K₂CO₃ (580 mg, 4.2 mmol) in DMF (15.0 mL) was stirred at 60° C. for 16 h. After cooled down to rt the mixture was diluted with EtOAc (50 mL), washed with water (20 mL*4), dried over anhydrous Na₂SO₄, filtered, concentrated and purified by prep-HPLC (C18 column, CH₃CN/H₂O, containing 0.05% NH₄HCO₃) to afford intermediate 3 (600 mg, yield 57%) as white solid. LCMS (m/z): 376.3 [M+H]⁺.

### tert-butyl 4-hydroxy-4-((6-(2-(4-methylpiperazin-1-yl)ethoxy)-4-oxoquinazolin-3(4H)-yl)methyl)piperidine-1-carboxylate (Intermediate 4)

A mixture of intermediate 3 (300 mg, 0.8 mmol), 1-(2-chloroethyl)-4-methylpiperazine (225 mg, 0.96 mmol), Cs₂CO₃ (521 mg, 1.6 mmol) and NaI (24 mg, 0.16 mmol) in DMAc (6.0 mL) was stirred at 120° C. for 16 h. The mixture was diluted with water (2.0 mL) and purified directly by prep-HPLC (C18 column, CH₃CN/H₂O, containing 0.05% NH₄HCO₃) to afford intermediate 3 (170 mg, yield 42%) as white solid. LCMS (m/z): 502.2 [M+H]⁺.

### 3-((4-hydroxypiperidin-4-yl)methyl)-6-(2-(4-methylpiperazin-1-yl)ethoxy)quinazolin-4(3H)-one (Intermediate 5)

To a mixture of intermediate 4 (170 mg, 0.339 mmol) in DCM (15 mL) was added HCl solution in 1,4-dioxane (2.0 mL, 4 M, 8.0 mmol), the mixture was stirred at room temperature for 2.5 hours and concentrated under vacuum to afford the target intermediate 5 (134 mg, 98% yield) as white solid. LCMS (m/z): 402.1 [M+H]⁺.

### N-(4-benzyl-5-(4-hydroxy-4-((6-(2-(4-methylpiperazin-1-yl)ethoxy)-4-oxoquinazolin-3(4H)-yl)methyl)piperidin-1-yl)-5-oxopentyl)-4-chloroquinoline-7-carboxamide (Compound 38)

A mixture of 2-benzyl-5-(4-chloroquinoline-7-carboxamido)pentanoic acid (0.0463 g, 0.1167 mmol, 1.0 eq), HATU (0.0524 g, 0.1378 mmol, 1.2 eq) and DIPEA (0.0459 g, 0.3552 mmol, 2.0 eq) in THF (4 mL) was stirred at room temperature for 1 hour, and then added dropwise to the mixture of intermediate 5 (0.1150 g, 0.2864 mmol, 1.2 eq), phenol (46.3 mg, 0.4920 mmol, 3.6 eq) and DIPEA (0.0665 g, 0.5146 mmol, 3.0 eq) in THF (4 mL), the mixture was stirred at room temperature for 2 hours. Water (100 mL) and DCM (100 mL) were added into the reaction mixture and layers separated. The aqueous phase was extracted with DCM (50 mL×3). The combined organic phase was washed with saturated NaCl solution (100 mL), dried over anhydrous Na₂SO₄, concentrated and purified by prep-HPLC (C18 column, CH₃CN/H₂O, containing 0.05% NH₄HCO₃) to afford the target compound 38 (14 mg, 15.38% yield) as white solid. ¹H NMR (400 MHz, DMSO-d₆) δ (ppm) 8.92 (d, J=4.4 Hz, 1H), 8.87 (t, J=5.6 Hz, 1H), 8.58 (d, J=10.8 Hz, 1H), 8.29-8.25 (m, 1H), 8.16 (t, J=10.4 Hz, 1H), 8.09-8.04 (m, 1H), 7.83 (d, J=4.4 Hz, 1H), 7.60 (d, J=8.8 Hz, 1H), 7.59-7.49 (m, 1H), 7.45-7.38 (m, 1H), 7.28-7.07 (m, 5H), 4.86 (br, 1H), 4.24-3.80 (m, 5H), 3.68-3.55 (m, 2H), 3.34-3.25 (m, 2H), 3.30-3.07 (m, 2H), 2.90-2.60 (m, 6H), 2.45-2.09 (m, 9H), 1.74-1.01 (m, 8H). LCMS (m/z): 780.3 [M+H]⁺.

### Synthesis of Compound 39

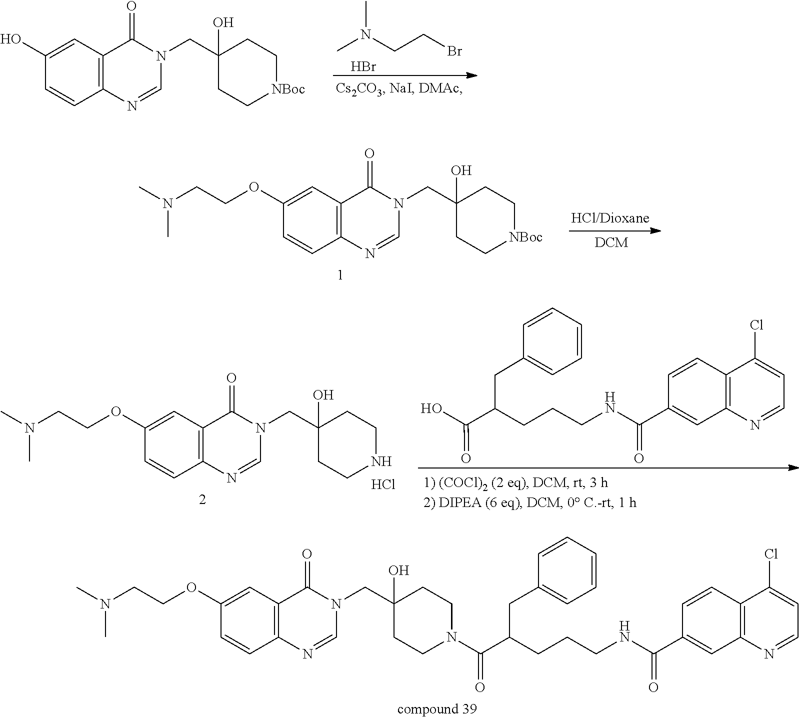

### tert-butyl 4-((6-(2-(dimethylamino)ethoxy)-4-oxoquinazolin-3(4H)-yl)methyl)-4-hydroxypiperidine-1-carboxylate (Intermediate 1)

A mixture of tert-butyl 4-hydroxy-4-((6-hydroxy-4-oxoquinazolin-3(4H)-yl)methyl)piperidine-1-carboxylate (275 mg, 0.73 mmol), 2-bromo-N,N-dimethylethanamine hydrobromide (172 mg, 0.74 mmol), Cs₂CO₃ (476 mg, 1.46 mmol) and NaI (25 mg, 0.17 mmol) in DMAC (6.0 mL) was stirred at 120° C. for 16 h. The mixture was diluted with water (30 mL) and extracted with DCM (20 mL×2), the combined organic was dried over anhydrous Na₂SO₄, concentrated and purified by prep-HPLC (C18 column, CH₃CN/H₂O, containing 0.05% NH₄HCO₃) to give intermediate 1 (91 mg, Yield 28%) as brown solid. LCMS (m/z): 447.3 [M+H]⁺.

### 6-(2-(dimethylamino)ethoxy)-3-((4-hydroxypiperidin-4-yl)methyl)quinazolin-4(3H)-one hydrochloride (Intermediate 2)

To a solution of N-(4-benzyl-5-(4-((6-(2-(dimethylamino)ethoxy)-4-oxoquinazolin-3(4H)-yl)methyl)-4-hydroxypiperidin-1-yl)-5-oxopentyl)-4-chloroquinoline-7-carboxamide (91 mg, 0.2 mmol) in DCM (3 mL) was added HCl solution in 1,4-Dioxane (4 M, 1 mL, 4 mmol) at rt. the mixture was stirred at rt for 3 hours and concentrated in vacuum to leave the crude intermediate 2 (77 mg, Yield 100%) as pale yellow solid. LCMS: (m/z) 347.2[M+H]⁺

### N-(4-benzyl-5-(4-((6-(2-(dimethylamino)ethoxy)-4-oxoquinazolin-3(4H)-yl)methyl)-4-hydroxypiperidin-1-yl)-5-oxopentyl)-4-chloroquinoline-7-carboxamide (Compound 39)

To a stirred solution of 2-benzyl-5-(4-chloroquinoline-7-carboxamido)pentanoic acid (38 mg, 0.096 mmol) in dry DCM (10 mL) was added dry DMF (0.05 mL), followed by addition of (COCl)₂ (18.3 mg, 0.144 mmol) at 0° C. The reaction mixture was stirred at rt for 3 hours and concentrated in vacuum, the residue was diluted with dry DCM (10 mL) and added dropwise to a mixture of 6-(2-(dimethylamino)ethoxy)-3-((4-hydroxypiperidin-4-yl)methyl)quinazolin-4(3H)-one hydrochloride (38.5 mg, 0.096 mmol) and DIEA (50 mg, 0.384 mmol) in dry DCM (10 mL). The resulting mixture was stirred at rt for 2 hours, concentrated and purified by prep-HPLC (C18 column, CH₃CN/H₂O, containing 0.05% NH₄HCO₃) to give product (18.5 mg, Yield 26.6%) as white solid. ¹H NMR (400 MHz, DMSO-d₆) δ (ppm) 8.93 (d, J=4.8 Hz, 1H), 8.88 (q, J=5.6 Hz, 1H), 8.60 (d, J=10.4 Hz, 1H), 8.30-8.24 (m, 1H), 8.20-8.13 (m, 1H), 8.11-8.05 (m, 1H), 7.85 (d, J=4.8 Hz, 1H), 7.64-7.49 (m, 2H), 7.45-7.37 (m, 1H), 7.28-7.06 (m, 5H), 4.83 (bs, 1H), 4.30-3.80 (m, 5H), 3.79-3.36 (m, 10H), 3.22-3.06 (m, 2H), 2.91-2.80 (m, 1H), 2.80-2.60 (m 4H), 2.23 (d, J=10.0 Hz, 6H), 1.75-1.01 (m, 8H). LCMS: (m/z) 725.3[M+H]⁺

### Synthesis of Compound 40

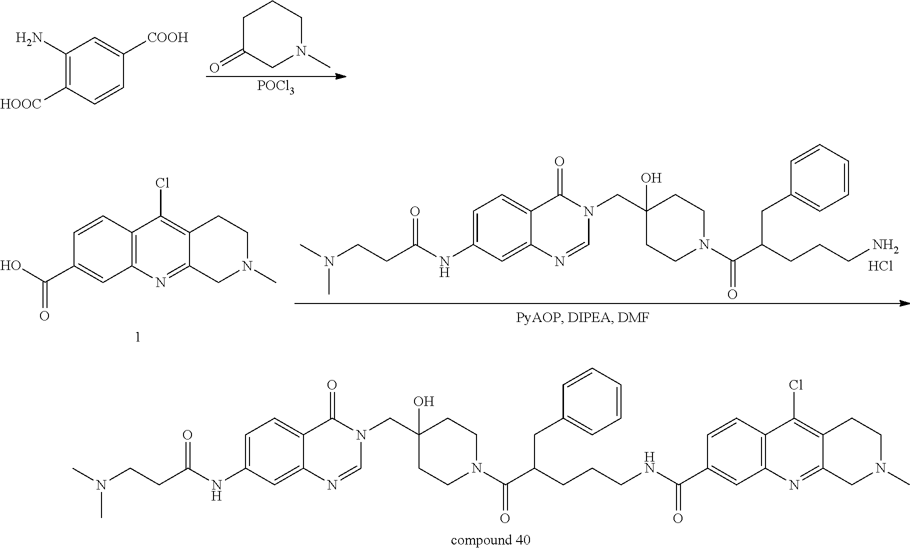

### 5-chloro-2-methyl-1,2,3,4-tetrahydrobenzo[b][1,7]naphthyridine-8-carboxylic acid (Intermediate 1)

A mixture of 2-aminoterephthalic acid (181 mg, 1.0 mmol) and 1-methylpiperidin-3-one (226 mg, 2.0 mmol) in POCl₃ (5 mL) was stirred at 100° C. for 30 minutes under microwave irradiation. After cooled down to rt the mixture was concentrated and purified by prep-HPLC (C18 column, CH₃CN/H₂O, containing 0.05% NH₄HCO₃) to give intermediate 1 (56 mg, Yield 18.4%) as white solid. LCMS (m/z): 277.1 [M+H]⁺.

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-5-chloro-2-methyl-1,2,3,4-tetrahydrobenzo[b][1,7]naphthyridine-8-carboxamide (Compound 40)

To a stirred solution of 5-chloro-2-methyl-1,2,3,4-tetrahydrobenzo[b][1,7]naphthyridine-8-carboxylic acid (30 mg, 0.108 mmol), N-(3-((1-(5-amino-2-benzylpentanoyl)-4-hydroxypiperidin-4-yl)methyl)-4-oxo-3,4-dihydroquinazolin-7-yl)-3-(dimethylamino)propanamide hydrochloride (65 mg, 0.108 mmol) and DIPEA (56 mg, 0.432 mmol) in dry DMF (5 mL) was added PyAOP (62 mg, 0.119 mmol). The reaction mixture was stirred at rt for 1 hour, diluted with EtOAc (100 mL), washed with Na₂CO₃ solution (50 mL) and brine (50 mL), dried over anhydrous Na₂SO₄, concentrated and purified by prep-HPLC (C18 column, CH₃CN/H₂O, containing 0.05% NH₄HCO₃) to give compound 40 (23 mg, Yield 26%) as white solid. ¹H NMR (400 MHz, DMSO-d₆) δ (ppm) 10.48 (d, J=6.0 Hz, 1H), 8.84-8.76 (m, 1H), 8.51-8.44 (m, 1H), 8.20-8.00 (m, 5H), 7.66-7.58 (m, 1H), 7.28-7.08 (m, 5H), 4.84 (d, J=2.8 Hz, 1H), 4.17-3.97 (m, 1H), 3.90-3.55 (m, 5H), 3.33-3.24 (m, 2H), 3.20-3.09 (m, 2H), 3.06-2.98 (m, 2H), 2.92-2.52 (m, 9H), 2.41 (d, J=9.2 Hz, 3H), 2.18 (s, 6H), 1.72-1.02 (m, 8H). LCMS: (m/z) 821.3 [M+H]⁺.

### Synthesis of Compound 44

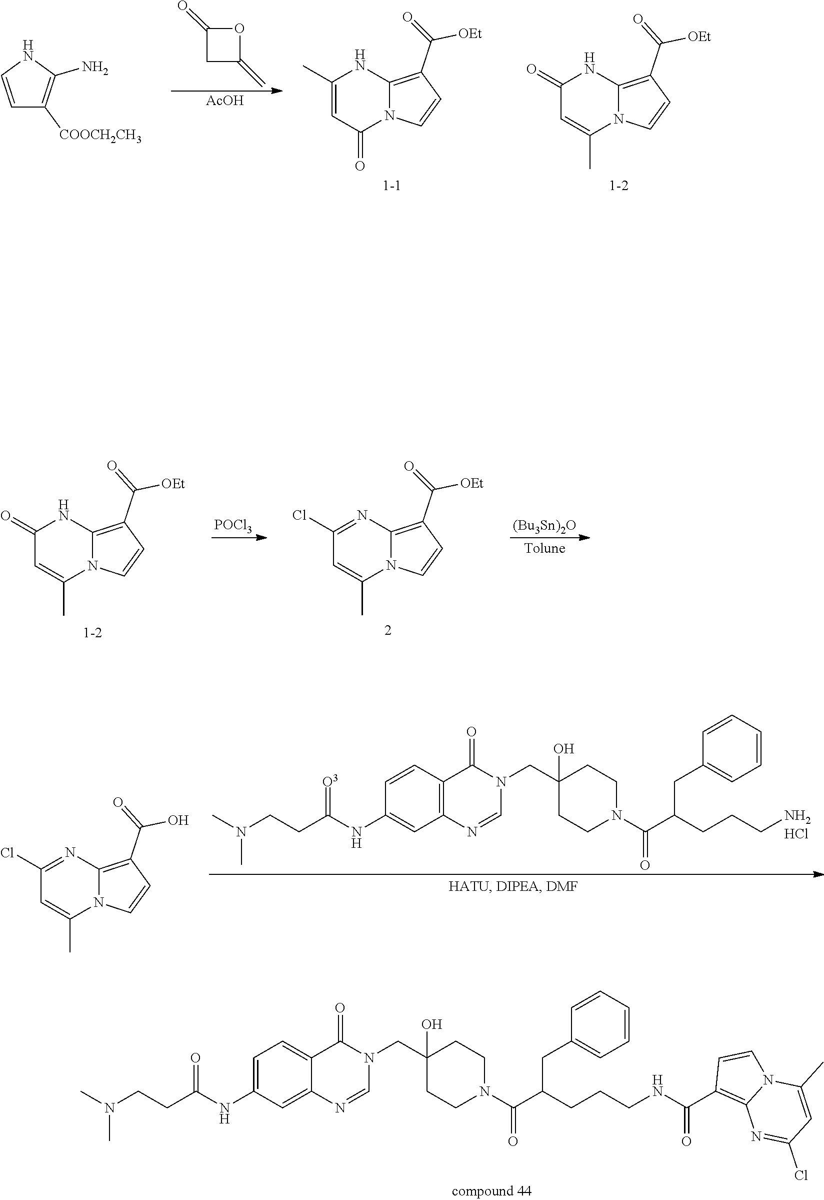

### 5-chloro-2-methyl-1,2,3,4-tetrahydrobenzo[b][1,7]naphthyridine-8-carboxylic acid (Intermediate 1)

To a mixture of ethyl 2-amino-1H-pyrrole-3-carboxylate (846 mg, 6.5 mmol) in HOAc (12 mL) was added 4-methyleneoxetan-2-one (1295 mg, 18.1 mmol). The mixture was stirred at 110° C. (preheated) for 2 hours. After cooled down to rt, the mixture was concentrated and purified by flash chromatograph (PE/EA=1/1) to give ethyl 2-methyl-4-oxo-1,4-dihydropyrrolo[1,2-a]pyrimidine-8-carboxylate (1-1, 530 mg, Yield 36.9%) as yellow solid, and ethyl 4-methyl-2-oxo-1,2-dihydropyrrolo[1,2-a]pyrimidine-8-carboxylate (1-2, 360 mg, Yield 25%) as red solid. LCMS (m/z): 1-2, 221.1 [M+H]⁺.

### Ethyl 2-chloro-4-methylpyrrolo[1,2-a]pyrimidine-8-carboxylate (Intermediate 2)

A mixture of ethyl 4-methyl-2-oxo-1,2-dihydropyrrolo[1,2-a]pyrimidine-8-carboxylate (120 mg, 0.54 mmol) in POCl₃ (5 mL) was stirred at 80° C. for 45 minutes under microwave irradiation. Upon cooling, the mixture was concentrated in vacuum, the residue was diluted with DCM (100 mL), adjusted to pH 7-8 with NaHCO₃ solution, the organics phase was separated, the aqueous was extracted with DCM (50 mL×2), the combined organics were washed with brine (50 mL), dried over anhydrous Na₂SO₄, filtered and concentrated to leave crude intermediate 2 (130 mg, Yield 99%) as brown solid. LCMS: (m/z) 239.1[M+H]⁺.

### 2-chloro-4-methylpyrrolo[1,2-a]pyrimidine-8-carboxylic acid (Intermediate 3)

A mixture of ethyl 2-chloro-4-methylpyrrolo[1,2-a]pyrimidine-8-carboxylate (82 mg, 0.343 mmol) and (Bu₃Sn)₂O (818 mg, 1.372 mmol) in dry Toluene (3 mL) was heated to reflux for 48 hours. Upon cooling, the mixture was concentrated in vacuum, the residue was diluted with EtOAc (50 mL) and extracted with saturated NaHCO₃ solution (50 mL×3). The combined aqueous phases were adjusted to pH 4-5 with 3 N HCl solution, and extracted with DCM (50 mL×3). The combined organics were dried over anhydrous Na₂SO₄, filtered and concentrated in vacuum to leave crude intermediate 3 (31.6 mg, Yield 43.9%) as yellow solid. LCMS: (m/z) 211.0[M+H]⁺.

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-2-chloro-4-methylpyrrolo[1,2-a]pyrimidine-8-carboxamide (Compound 44)

To a stirring solution of 2-chloro-4-methylpyrrolo[1,2-a]pyrimidine-8-carboxylic acid (31.6 mg, 0.15 mmol), N-(3-((1-(5-amino-2-benzylpentanoyl)-4-hydroxypiperidin-4-yl)methyl)-4-oxo-3,4-dihydroquinazolin-7-yl)-3-(dimethylamino)propanamide hydrochloride (90 mg, 0.15 mmol) and DIPEA (78 mg, 0.6 mmol) in DMF (5 mL) was added HATU (63 mg, 0.17 mmol). The mixture was stirred at rt for 1 hour, diluted with EtOAc (100 mL), washed with aq. NaHCO₃ (50 mL) and brine (50 mL), dried over anhydrous Na₂SO₄, filtered, concentrated and purified by prep-HPLC (C18 column, CH₃CN/H₂O, containing 0.05% NH₄HCO₃) to give compound 44 (24.8 mg, Yield 22%) as white solid. ¹H NMR (400 MHz, DMSO-d₆) δ (ppm) 10.48 (d, J=5.2 Hz, 1H), 8.16-7.99 (m, 3H), 7.92-7.86 (m, 1H), 7.65-7.58 (m, 1H), 7.54-7.50 (m, 1H), 7.38-7.33 (m, 1H), 7.25-7.06 (m, 5H), 7.02 (d, J=2.8 Hz, 1H), 4.82 (s, 1H), 4.15-3.96 (m, 1H), 3.96-3.54 (m, 4H), 3.19-3.06 (m, 2H), 2.89-2.63 (m, 7H), 2.62-2.52 (m, 4H), 2.19 (s, 6H), 1.71-0.98 (m, 8H). LCMS: (m/z) 755.9[M+H]⁺.

### Synthesis of Compound 45

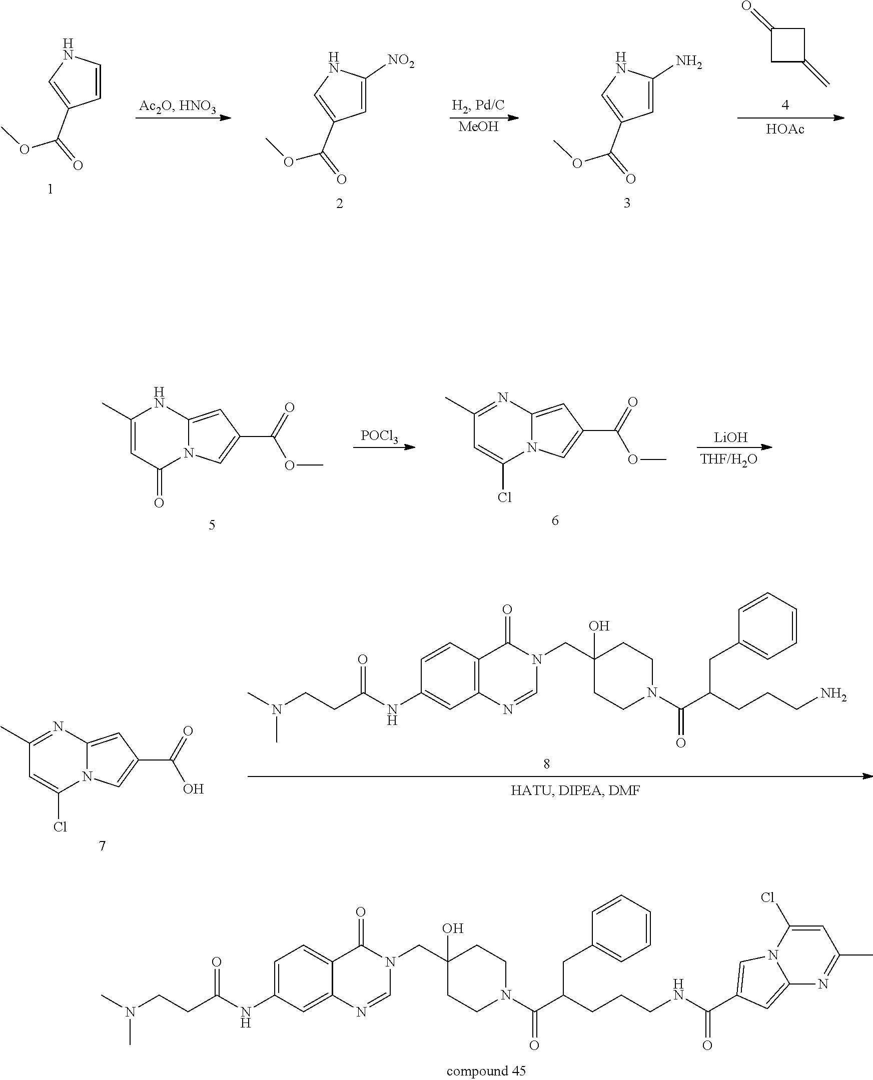

### methyl 5-nitro-1H-pyrrole-3-carboxylate (Intermediate 2)

The solution of methyl 1H-pyrrole-3-carboxylate (500 mg, 4.00 mmol) in acetic anhydride (4.8 mL) was added dropwise to a mixture of nitric acid (70%, 1.0 mL) and acetic anhydride (2.4 mL) at 50° C. The mixture was stirred at 60° C. overnight. After cooled down to rt, the reaction mixture was poured into crushed ice, the mixture was extracted with EtOAc (50 mL), dried over anhydrous Na₂SO₄, concentrated and purified by flash column chromatography (ethyl acetate:petroleum ether=1:1) to obtain intermediate 2 (130 mg, Yield 19.1%) as yellow solid. LCMS (m/z): 171.7 [M+1]⁺.

### methyl 5-amino-1H-pyrrole-3-carboxylate (Intermediate 3)

To a solution of methyl 5-nitro-1H-pyrrole-3-carboxylate (130.0 mg, 0.76 mmol) in MeOH (10 mL) was added Pd/C (10%, 13 mg), the mixture was stirred at rt under H₂ (1 atm) overnight and filtered through celite, the filtrate was concentrated in vacuum to obtain intermediate 3 (80 mg, Yield 74.8%) as grey solid. LCMS (m/z): 141.1 [M+H]⁺.

### methyl 2-methyl-4-oxo-1,4-dihydropyrrolo[1,2-a]pyrimidine-7-carboxylate (Intermediate 5)

To a solution of methyl 5-amino-1H-pyrrole-3-carboxylate (80.0 mg, 0.57 mmol) in HOAc (10 mL) was added 3-methylenecyclobutan-1-one (134.4 mg, 1.60 mmol) in one portion. The reaction mixture was stirred at 110° C. for 2 hours and concentrated in vacuum, the residue was purified by flash column chromatography on silica gel (PE/EA: 20˜10:1) to afford intermediate 5 (70 mg, Yield 59.5%) as white solid. LCMS: (m/z) 207.1 [M+H]⁺.

### methyl 4-chloro-2-methylpyrrolo[1,2-a]pyrimidine-7-carboxylate (Intermediate 6)

A mixture of methyl 2-methyl-4-oxo-1,4-dihydropyrrolo[1,2-a]pyrimidine-7-carboxylate (70.0 mg, 0.34 mmol) in POCl₃ (5 mL) was heated at 80° C. for 1 hour. The reaction mixture was concentrated in vacuum, the residue was adjusted to pH 6-7 with saturated NaHCO₃ solution and extracted with ethyl acetate (20 mL*3), the combined organic was washed with brine (50 mL), dried over anhydrous Na₂SO₄, filtered and concentrated to leave crude intermediate 6 (70 mg, Yield 92.1%) as white solid. LCMS: (m/z) 225.1 [M+H]⁺.

### 4-chloro-2-methylpyrrolo[1,2-a]pyrimidine-7-carboxylic acid (Intermediate 7)

A solution of LiOH (11.2 mg, 0.312 mmol) in H₂O (5 mL) was added to the solution of methyl 4-chloro-2-methylpyrrolo[1,2-a]pyrimidine-7-carboxylate (70.0 mg, 0.312 mmol) in THF (5 mL), the mixture was stirred at rt overnight, adjusted to pH 1-2 with 1 N HCl solution and extracted with ethyl acetate (20 mL×3), the combined organic was washed with brine (50 mL), dried over anhydrous Na₂SO₄, filtered and concentrated to leave intermediate 7 (56 mg, Yield 85.4%) as white solid. LCMS: (m/z) 211.1 [M+H]⁺.

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-2-methylpyrrolo[1,2-a]pyrimidine-7-carboxamide (Compound 45)

To a mixture of 4-chloro-2-methylpyrrolo[1,2-a]pyrimidine-7-carboxylic acid (20.1 mg, 0.10 mmol), N-(3-((1-(5-amino-2-benzylpentanoyl)-4-hydroxypiperidin-4-yl)methyl)-4-oxo-3,4-dihydroquinazolin-7-yl)-3-(dimethylamino)propanamide (56.3 mg, 0.10 mmol) and DIPEA (41.5 mg, 0.30 mmol) in DMF (10 mL) was added HATU (41.8 mg, 0.11 mmol). The mixture was stirred at rt for 30 minutes, diluted with EtOAc (80 mL), washed with saturated NaHCO₃ solution (5 mL), dried over anhydrous Na₂SO₄, filtered, concentrated and purified by prep HPLC (C18 column, CH₃CN/H₂O, containing 0.05% NH₄HCO₃) to obtain compound 45 (15 mg, Yield 20.0%) as white solid. ¹H NMR (400 MHz, DMSO-d₆) δ (ppm) 10.48 (d, J=4.4 Hz, 1H), 8.35 (dt, J=11.3, 5.8 Hz, 1H), 8.18-7.99 (m, 3H), 7.88 (dd, J=15.6, 1.5 Hz, 1H), 7.62 (td, J=9.0, 2.0 Hz, 1H), 7.27-7.19 (m, 2H), 7.17-7.08 (m, 3H), 6.91 (dd, J=13.0, 1.5 Hz, 1H), 6.83 (s, 1H), 4.85 (s, 1H), 4.13 (d, J=12.5 Hz, 1H), 4.04-3.76 (m, 2H), 3.63 (d, J=13.6 Hz, 1H), 3.25 (d, J=6.0 Hz, 2H), 3.11 (d, J=10.5 Hz, 2H), 2.85 (t, J=10.7 Hz, 1H), 2.70 (ddd, J=17.2, 12.9, 6.8 Hz, 3H), 2.60 (t, J=5.8 Hz, 5H), 2.54 (s, 1H), 2.19 (s, 6H), 1.69-1.56 (m, 1H), 1.45 (dd, J=17.0, 13.2 Hz, 4H), 1.33-1.05 (m, 3H). LCMS (m/z): 756.3 [M+H]⁺.

### Synthesis of Compound 47

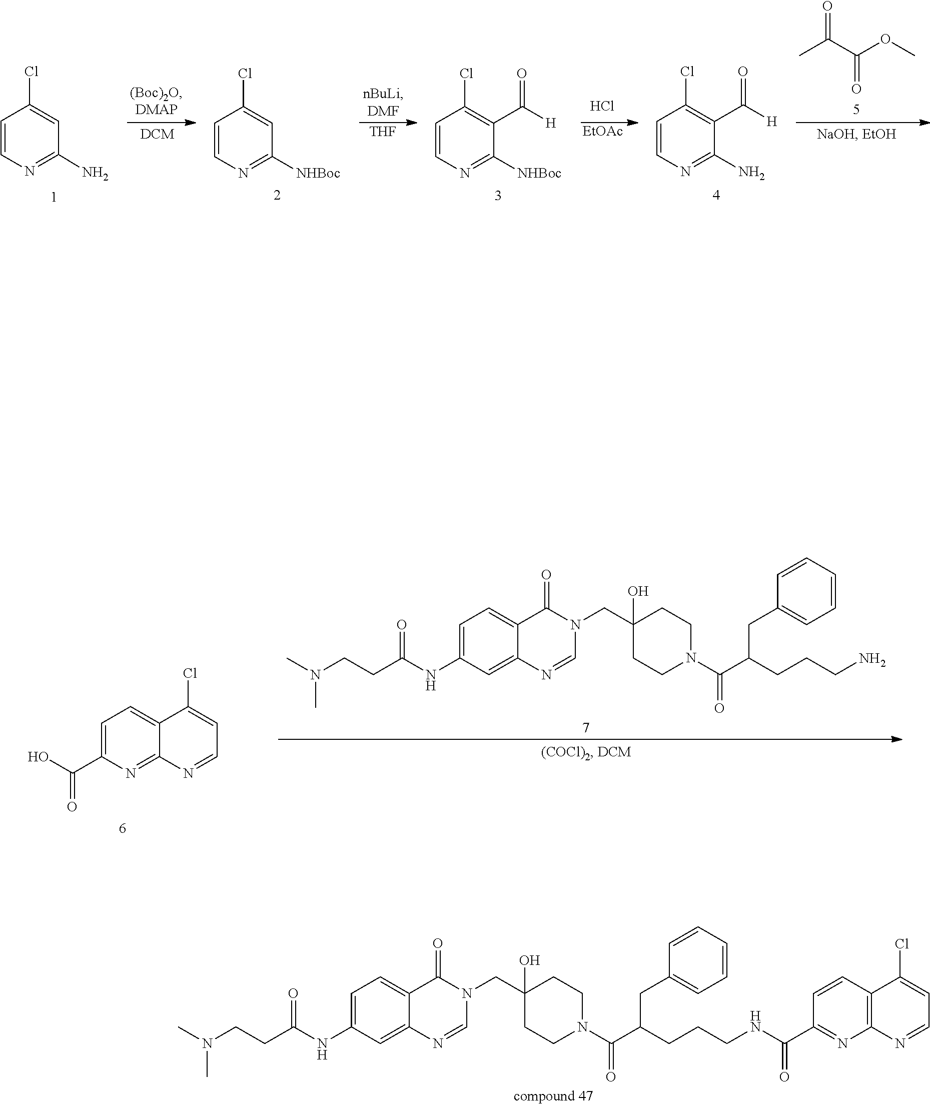

### tert-butyl (4-chloropyridin-2-yl)carbamate (Intermediate 2)

To the solution of 4-chloropyridin-2-amine (3.0 g, 23.3 mmol) in DCM (100 mL) was added di-tert-butyl dicarbonate (5.6 g, 25.7 mmol), followed by slow addition of DMAP (0.57 g, 4.66 mmol). Vigorous bubbling persisted for about 1 h after which the reaction was concentrated in vacuo. The residue was purified by flash column chromatography on silica gel (PE/EA:20˜10:1) to afford intermediate 2 (2.8 g, yield 52.4%) as white solid. LCMS (m/z): 173.1 [M-55]⁺

### tert-butyl (4-chloro-3-formylpyridin-2-yl)carbamate (Intermediate 3)

To a solution of tert-butyl (4-chloropyridin-2-yl)carbamate (1.8 g, 7.87 mmol) in THF (30 mL) at −60° C. was added dropwise n-butyllithium solution (2.5 M in hexanes, 7.4 mL, 18.50 mmol). The solution was stirred at −60° C. for 1 h, and then DMF (2.7 g, 36.99 mmol) was added dropwise. After stirred at −60° C. for 1 hour, the reaction mixture was quenched by the dropwise addition of saturated ammonium chloride solution (50 mL). The mixture was extracted with ethyl acetate (50 mL×3), the combined organic were dried over anhydrous MgSO₄, filtered, concentrated and purified by column chromatography (elution with ethyl acetate/PE, 1:10) to give intermediate 3 (1 g, yield 49.5%) as white solid. LCMS (m/z): 201.0 [M-55]⁺

### 2-amino-4-chloronicotinaldehyde (Intermediate 4)

To tert-butyl (4-chloro-3-formylpyridin-2-yl)carbamate (1.0 g, 3.90 mmol) was added HCl solution in EtOAc (20 mL), the mixture was stirred at rt overnight and filtered, the cake was dried under vacuum to obtain intermediate 4 (600 mg, yield 98.5%) as white solid. LCMS: (m/z) 157.1 [M+H]⁺

### 5-chloro-1,8-naphthyridine-2-carboxylic acid (Intermediate 6)

To the mixture of 2-amino-4-chloronicotinaldehyde (450 mg, 2.87 mmol) and methyl 2-oxopropanoate (322.5 mg, 5.75 mmol) in EtOH (10 mL) and H₂O (2 mL) was added NaOH solution (420.2 mg, 10.5 mmol) in H₂O (10 mL). The mixture was stirred at rt for another 3 hours, adjusted to pH 1-2 with 1 N HCl solution and concentrated under reduced pressure, the residue was extracted with ethyl acetate (20 mL×3), the combined organic was washed with brine (50 mL), dried over anhydrous Na₂SO₄, filtered and concentrated under vacuum to obtain intermediate 6 (280 mg, yield 46.7%) as white solid. LCMS: (m/z) 291.1 [M+Na]⁺.

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-5-chloro-1,8-naphthyridine-2-carboxamide (Compound 47)

To a mixture of 5-chloro-1,8-naphthyridine-2-carboxylic acid (40 mg, 0.192 mmol) in dry DCM (5 mL) was added one drop of DMF at 0° C. under N₂, and then (COCl)₂ (36.5 mg, 0.290 mmol) was added, the mixture was stirred at 0° C. for 10 minutes, then warm to room temperature for another 1 hours. The mixture was added dropwise to a mixture of N-(3-((1-(5-amino-2-benzylpentanoyl)-4-hydroxypiperidin-4-yl)methyl)-4-oxo-3,4-dihydroquinazolin-7-yl)-3-(dimethylamino)propanamide (70 mg, 0.124 mmol) in dry DCM (5 mL) at 0-5° C., and then warm to room temperature for another 1 hour. The reaction mixture was diluted with DCM (80 mL), washed with saturated NaHCO₃ solution (5 mL), dried over anhydrous Na₂SO₄, concentrated and purified by column chromatographic (DCM: MeOH 10:1) to obtain compound 47 (14.1 mg, yield 18.6%) as white solid. ¹H NMR (400 MHz, DMSO-d₆) δ 10.51 (d, J=6.5 Hz, 1H), 9.15 (t, J=4.5 Hz, 2H), 8.85 (dd, J=8.6, 2.8 Hz, 1H), 8.39 (dd, J=11.9, 8.6 Hz, 1H), 8.19-7.90 (m, 4H), 7.62 (dd, J=11.5, 9.4 Hz, 1H), 7.32-6.98 (m, 5H), 4.82 (s, 1H), 4.17-3.74 (m, 4H), 3.64-3.60 (m, 2H), 3.16 (s, 2H), 2.82-2.65 (m, 6H), 2.57 (s, 1H), 2.30 (s, 6H), 1.75-1.30 (m, 6H), 1.2-1.1 (m, 2H). LCMS (m/z): 753.3 [M+H]⁺.

### Synthesis of Compound 48

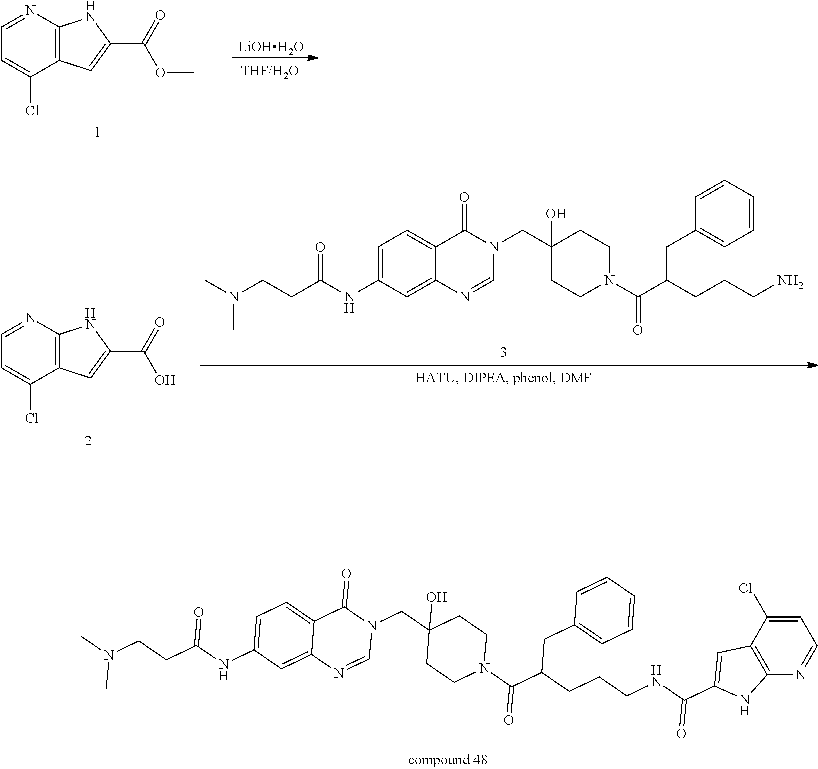

### 4-chloro-1H-pyrrolo[2,3-b]pyridine-2-carboxylic acid (2)

A solution of LiOH (17.1 mg, 0.71 mmol) in H₂O (5 mL) was added to a solution of methyl 4-chloro-1H-pyrrolo[2,3-b]pyridine-2-carboxylate (100.0 mg, 0.47 mmol) in THF (5 mL), the mixture was stirred at rt overnight, adjusted to pH 1-2 with 1 N HCl solution and concentrated to leave crude intermediate 2 (110 mg, crude) as white solid, which was used directly in the next step. LCMS (m/z): 197.1 [M+H]⁺.

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-1H-pyrrolo[2,3-b]pyridine-2-carboxamide (Compound 48)

To a mixture of N-(3-((1-(5-amino-2-benzylpentanoyl)-4-hydroxypiperidin-4-yl)methyl)-4-oxo-3,4-dihydroquinazolin-7-yl)-3-(dimethylamino)propanamide (69.0 mg, 0.12 mmol), 4-chloro-1H-pyrrolo[2,3-b]pyridine-2-carboxylic acid (40.1 mg, 0.20 mmol), DIPEA (41.5 mg, 0.30 mmol) and phenol (28.2 mg, 0.30 mmol) in DMF (10 mL) was added HATU (41.8 mg, 0.11 mmol), the mixture was stirred at rt for 30 minutes, diluted with saturated NaHCO₃ solution (30 mL) and extracted with ethyl acetate (20 mL×3), the combined organic was washed with brine (50 mL), dried over anhydrous Na₂SO₄, concentrated and purified by prep HPLC to obtain compound 48 (5.9 mg, yield 7.96%) as white solid. ¹H NMR (400 MHz, DMSO-d₆) δ (ppm) 12.49 (s, 1H), 10.65 (d, J=4.2 Hz, 1H), 9.40 (s, 1H), 8.63 (dd, J=12.1, 6.1 Hz, 1H), 8.33-7.96 (m, 4H), 7.68-7.57 (m, 1H), 7.26 (d, J=5.2 Hz, 1H), 7.21 (dd, J=9.9, 4.6 Hz, 2H), 7.15 (dd, J=12.3, 4.8 Hz, 2H), 7.09 (dd, J=13.8, 6.6 Hz, 1H), 4.82 (s, 1H), 4.17-3.91 (m, 2H), 3.82 (d, J=13.6 Hz, 1H), 3.63 (d, J=13.7 Hz, 2H), 3.41 (d, J=5.2 Hz, 2H), 3.25 (d, J=5.6 Hz, 2H), 3.15 (d, J=11.0 Hz, 2H), 2.91 (t, J=6.9 Hz, 2H), 2.83 (d, J=4.3 Hz, 6H), 2.75-2.71 (m, 1H), 2.67 (dd, J=12.7, 5.1 Hz, 1H), 1.65-1.55 (m, 1H), 1.55-1.35 (m, 4H), 1.33-1.06 (m, 3H). LCMS (m/z): 741.3 [M+H]⁺.

### Synthesis of Compound 49, 50, and 51

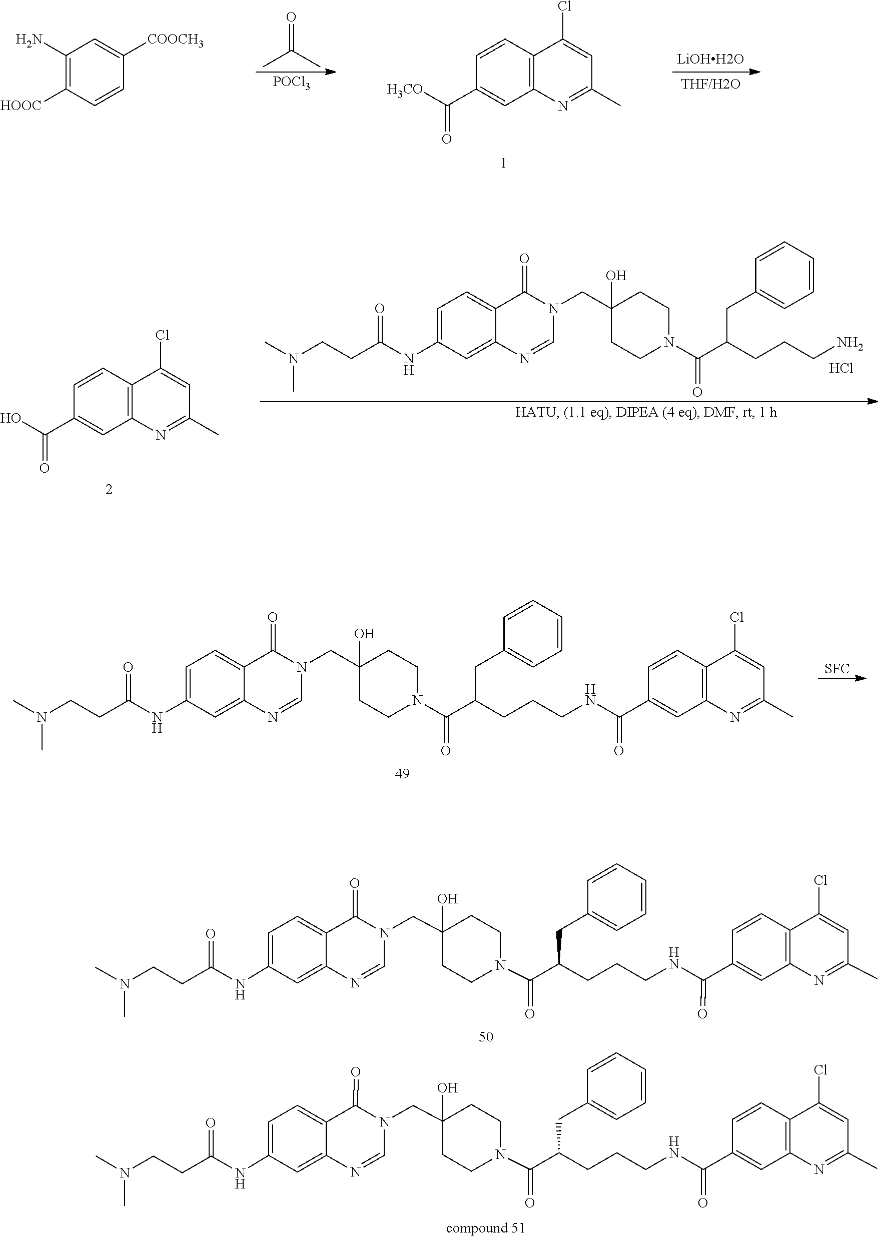

### 5-chloro-2-methyl-1,2,3,4-tetrahydrobenzo[b][1,7]naphthyridine-8-carboxylic acid (Intermediate 1)

A mixture of 2-amino-4-(methoxycarbonyl)benzoic acid (605 mg, 3.1 mmol) and acetone (1.8 g, 31 mmol) in POCl₃ (10 mL) was stirred at 100° C. for 30 minutes under microwave irradiation. The mixture was concentrated in vacuum, the residue was diluted with DCM (100 mL), adjusted to pH 7-8 with Na₂CO₃ solution, and extracted with DCM (50 mL×3). The combined organics was washed with brine (100 mL), dried over anhydrous Na₂SO₄, filtered, concentrated and purified by flash column chromatograph (DCM/MeOH=30/1) to give intermediate 1 (237 mg, yield 32.2%) as brown solid. LCMS (m/z): 236.1 [M+H]⁺.

### 4-chloro-2-methylquinoline-7-carboxylic acid (Intermediate 2)

To a mixture of methyl 4-chloro-2-methylquinoline-7-carboxylate (237 mg, 1.0 mmol) in THF/H₂O (10 mL/2 mL) was added LiOH·H₂O (63 mg, 1.5 mmol). The mixture was stirred at rt for 16 hours, and then HCl solution in 1,4-Dioxane (4 M, 0.4 mL, 1.6 mmol) was added, the mixture was concentrated to leave crude intermediate 2 (280 mg, yield 100%) as yellow solid. LCMS: (m/z) 222.0[M+H]⁺,

### N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-2-methylquinoline-7-carboxamide (Compound 49)

To a stirred solution of 4-chloro-2-methylquinoline-7-carboxylic acid (28 mg, crude 0.1 mmol), N-(3-((1-(5-amino-2-benzylpentanoyl)-4-hydroxypiperidin-4-yl)methyl)-4-oxo-3,4-dihydroquinazolin-7-yl)-3-(dimethylamino)propanamide hydrochloride (60 mg, 0.1 mmol) and DIPEA (52 mg, 0.4 mmol) in DMF (5 mL) was added HATU (42 mg, 0.11 mmol). The mixture was stirred at rt for 1 hour, and then purified directly by prep-HPLC ((C18 column, CH₃CN/H₂O, containing 0.05% TFA) to give compound 49 (33.9 mg, Yield 44%) as solid. ¹H NMR (400 MHz, DMSO-d₆) δ (ppm) 10.71 (d, J=4.4 Hz, 1H), 9.53 (s, 1H), 8.87-8.79 (m, 1H), 8.53-8.47 (m, 1H), 8.23-8.01 (m, 5H), 7.78 (d, J=2.4 Hz, 1H), 7.68-7.59 (m, 1H), 7.27-7.06 (m, 5H), 4.18-3.88 (m, 4H), 3.67-3.52 (m, 3H), 3.46-3.37 (m, 2H), 3.35-3.24 (m, 2H), 3.21-3.08 (m, 2H), 2.98-2.89 (m, 2H), 2.84 (d, J=4.0 Hz, 6H), 2.79-2.59 (m, 5H), 1.77-0.99 (m, 7H). LCMS: (m/z) 766.3[M+H]⁺.

### (R)—N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-2-methylquinoline-7-carboxamide (Compound 50)

### (S)—N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-2-methylquinoline-7-carboxamide (Compound 51)

N-(4-benzyl-5-(4-((7-(3-(dimethylamino)propanamido)-4-oxoquinazolin-3(4H)-yl)methyl)-4-hydroxypiperidin-1-yl)-5-oxopentyl)-4-chloro-2-methylquinoline-7-carboxamide (21 mg, 0.1 mmol) was purified by Chiral HPLC (Instrument: Gilson-281, Column: IC 20*250, 10 um; Mobile Phase: MEOH (0.2% Methanol Ammonia):CAN (0.2% Methanol Ammonia)=50:50; Flow Rate: 50 mL/min; Run time per injection: 26 min; Injection: 1 ml; Sample solution: 20 mg in 3 mL MEOH) to give the 2 enantiomers compound 50 (8.5 mg), LCMS: (m/z) 766.3[M+H]⁺, and compound 51 (8.5 mg), LCMS: (m/z) 766.3[M+H]⁺.

### Synthesis of Compound 52

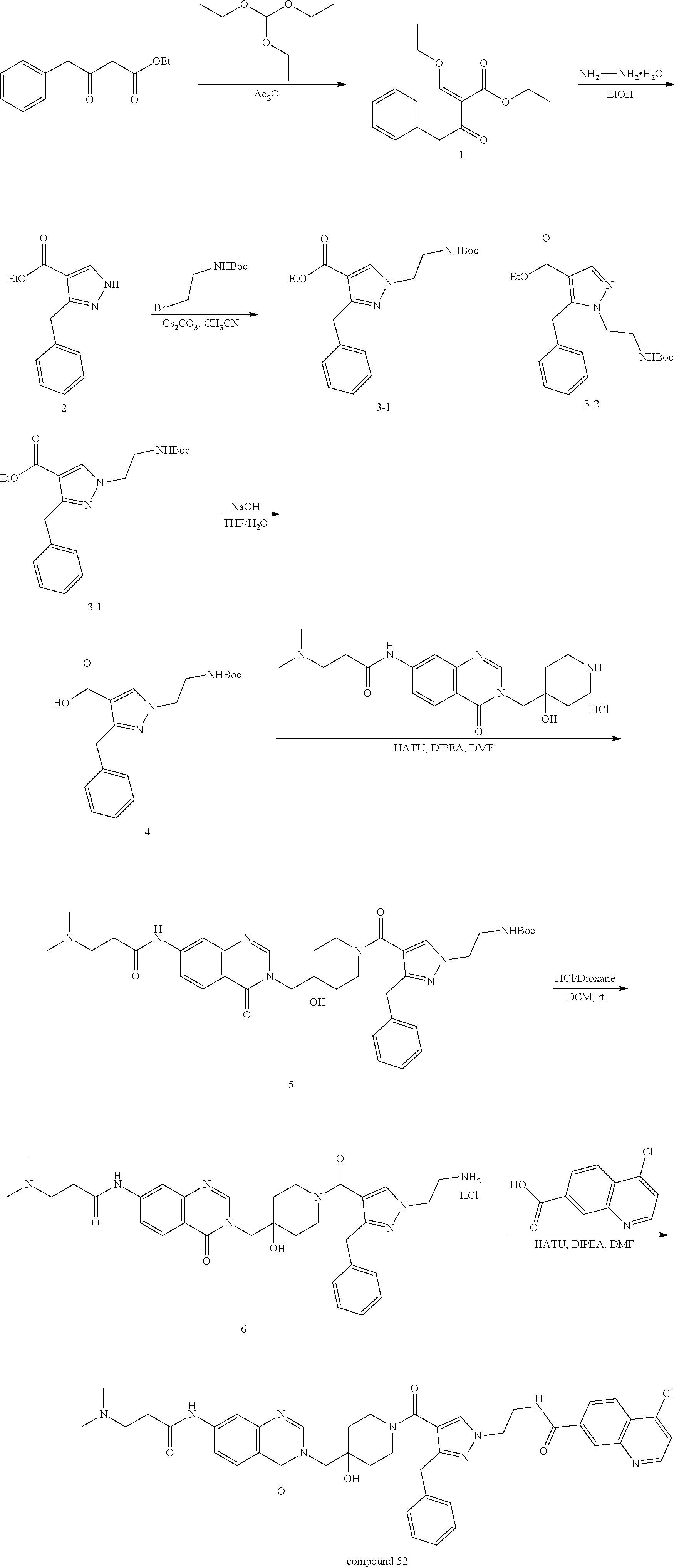

### (Z)-ethyl 2-(ethoxymethylene)-3-oxo-4-phenylbutanoate (Intermediate 1)

A mixture of ethyl 3-oxo-4-phenylbutanoate (2062 mg, 10 mmol), triethoxymethane (4456 mg, 30 mmol) and Ac₂O (1633 mg, 16 mmol) was stirred at 100° C. for 17 hours. Upon cooling, the mixture was diluted with EtOAc (100 mL) and H₂O (100 mL), the mixture was stirred at rt for 10 minutes, the organic phase was separated, washed with aqueous NaHCO₃ solution (50 mL) and brine (50 mL), dried over anhydrous Na₂SO₄, filtered and concentrated to leave crude intermediate 1 (2.62 g) as red oil. LCMS (m/z): 263.1 [M+H]⁺.

### Ethyl 3-benzyl-1H-pyrazole-4-carboxylate (Intermediate 2)

To a stirred solution of (Z)-ethyl 2-(ethoxymethylene)-3-oxo-4-phenylbutanoate (2358 mg, 9.0 mmol) in dry EtOH (30 mL) was added NH₂—NH₂·H₂O (85%, 583 mg, 9.9 mmol) at 0° C. The mixture was stirred at rt for 12 h, the mixture was concentrated in vacuum, the residue was diluted with EtOAc (100 mL), washed with H₂O (100 mL), dried over anhydrous Na₂SO₄, filtered, concentrated and purified by flash column chromatograph (PE/EA=1/1) to give intermediate 2 (1.346 g, yield 65%) as solid. LCMS: (m/z) 231.1[M+H]⁺,

### Ethyl 3-benzyl-1-(2-(tert-butoxycarbonylamino)ethyl)-1H-pyrazole-4-carboxylate (Intermediate 3-1)

### Ethyl 5-benzyl-1-(2-(tert-butoxycarbonylamino)ethyl)-1H-pyrazole-4-carboxylate (Intermediate 3-2)

To a mixture of ethyl 3-benzyl-1H-pyrazole-4-carboxylate (1.26 g, 5.47 mmol) and Cs₂CO₃ (3.564 g, 10.94 mmol) in CH₃CN (40 mL) was added tert-butyl 2-bromoethylcarbamate (1.838 g, 8.20 mmol). The mixture was stirred at rt for 16 hours and filtered. The solid was washed with DCM, the combined filtrate and washing was concentrated and purified by flash column chromatograph (DCM/CH₃OH=10/1) to give ethyl 3-benzyl-1-(2-(tert-butoxycarbonylamino)ethyl)-1H-pyrazole-4-carboxylate (3-1, 920 mg, Yield 38%) and ethyl 5-benzyl-1-(2-(tert-butoxycarbonylamino)ethyl)-1H-pyrazole-4-carboxylate (3-2, 430 mg, yield 17.8%). LCMS: 3-1, (m/z) 374.0[M+H]⁺, 3-2, (m/z) 374.0[M+H]⁺,

### 3-benzyl-1-(2-(tert-butoxycarbonylamino)ethyl)-1H-pyrazole-4-carboxylic acid (Intermediate 4)

A mixture of ethyl 3-benzyl-1-(2-(tert-butoxycarbonylamino)ethyl)-1H-pyrazole-4-carboxylate (460 mg, 1.23 mmol) and NaOH (197 mg, 4.92 mmol) in THF/H₂O (10 mL/2 mL) was heated to reflux for 16 hours. The mixture was cooled down to rt, acidified with 1 N HCl solution and extracted with EtOAc (50 mL). The organic was dried over anhydrous Na₂SO₄, filtered and concentrated to leave crude intermediate 4 (390 mg, yield 92%) as white solid. LCMS: (m/z) 346.2[M+H]⁺.

### Tert-butyl 2-(3-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethylcarbamate (Intermediate 5)

To a stirred solution of 3-benzyl-1-(2-(tert-butoxycarbonylamino)ethyl)-1H-pyrazole-4-carboxylic acid (138 mg, 0.4 mmol), 3-(dimethylamino)-N-(3-((4-hydroxypiperidin-4-yl)methyl)-4-oxo-3,4-dihydroquinazolin-7-yl)propanamide hydrochloride (164 mg, 0.4 mmol) and DIPEA (207 mg, 1.6 mmol) in DMF (6 mL) was added HATU (167 mg, 0.44 mmol). The mixture was stirred at rt for 1 hour, diluted with EtOAc (100 mL), washed with H₂O (50 mL), dried over anhydrous Na₂SO₄, filtered and concentrated in vacuum. The residue was purified by flash column chromatograph (DCM/MeOH=10/1) to give intermediate 5 (216 mg, yield 77%) as white solid. LCMS: (m/z) 701.4 [M+H]⁺.

### N-(3-((1-(1-(2-aminoethyl)-3-benzyl-1H-pyrazole-4-carbonyl)-4-hydroxypiperidin-4-yl)methyl)-4-oxo-3,4-dihydroquinazolin-7-yl)-3-(dimethylamino)propanamide hydrochloride (Intermediate 6)

To a solution of tert-butyl 2-(3-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethylcarbamate (216 mg, 0.308 mmol) in DCM (10 mL) was added HCl solution in 1,4-Dioxane (4 M, 1.5 mL, 6.0 mmol). The mixture was stirred at rt for 3 h and concentrated to give intermediate 6 (220 mg, yield 100%) as HCl salt. LCMS: (m/z) 601.3 [M+H]⁺.

### N-(2-(3-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethyl)-4-chloroquinoline-7-carboxamide (Compound 52)

To a stirred solution of N-(3-((1-(1-(2-aminoethyl)-3-benzyl-1H-pyrazole-4-carbonyl)-4-hydroxypiperidin-4-yl)methyl)-4-oxo-3,4-dihydroquinazolin-7-yl)-3-(dimethylamino)propanamide hydrochloride (74 mg, 0.116 mmol), 4-chloroquinoline-7-carboxylic acid (24 mg, 0.116 mmol) and DIPEA (60 mg, 0.464 mmol) in DMF (6 mL) was added HATU (48 mg, 0.128 mmol). The mixture was stirred at rt for 1 hour, diluted with EtOAc (100 mL), washed with H₂O (50 mL), dried over anhydrous Na₂SO₄, filtered and concentrated in vacuum. The residue was purified by prep-HPLC (C18 column, CH₃CN/H₂O, containing 0.05% NH₄HCO₃) to give compound 52 (18.8 mg, yield 20.5%) as white solid. ¹H NMR (400 MHz, DMSO-d₆) δ (ppm) 10.47 (s, 1H), 9.00 (t, J=5.6 Hz, 1H), 8.88 (d, J=4.8 Hz, 1H), 8.54 (d, J=1.2 Hz, 1H), 8.23 (d, J=8.4 Hz, 1H), 8.15 (s, 1H), 8.11-8.03 (m, 3H), 7.84 (s, 1H), 7.82 (d, J=4.8 Hz, 1H), 7.61 (dd, J=2.0 Hz, 8.8 Hz, 1H), 7.19-7.05 (m, 5H), 4.87 (s, 1H), 3.30 (t, J=6.0 Hz, 2H), 3.96-3.69 (m, 8H), 3.01 (bs, 2H), 2.61-2.52 (m, 4H), 2.18 (s, 6H), 1.22 (bs, 4H). LCMS: (m/z) 790.3 [M+H]⁺.

### Synthesis of Compound 53

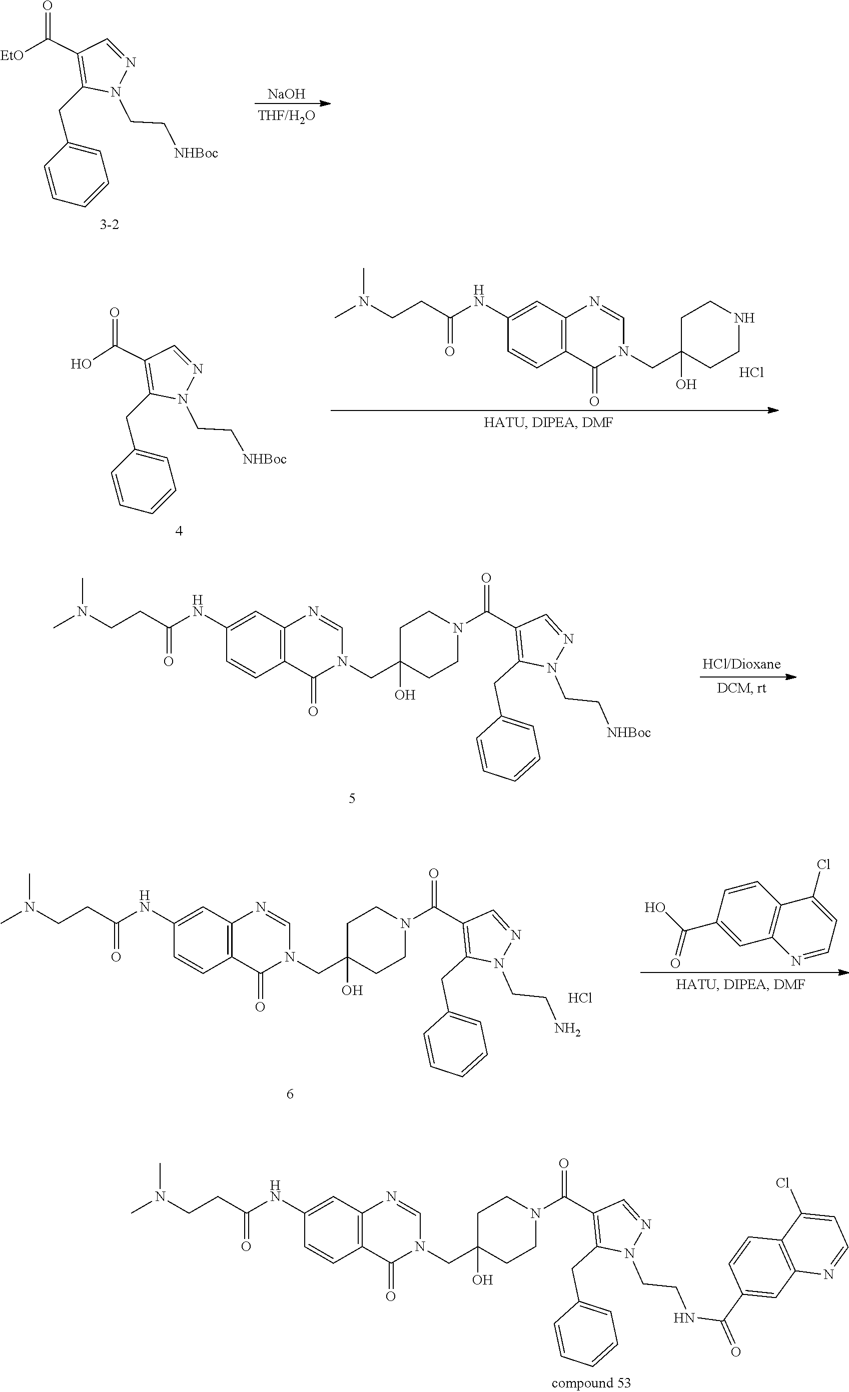

### 5-benzyl-1-(2-(tert-butoxycarbonylamino)ethyl)-1H-pyrazole-4-carboxylic acid (Intermediate 4)

A mixture of ethyl 5-benzyl-1-(2-(tert-butoxycarbonylamino)ethyl)-1H-pyrazole-4-carboxylate (430 mg, 1.15 mmol) and NaOH (184 mg, 4.60 mmol) in THF/H₂O (10/2 mL) was stirred at reflux for 16 hours. The mixture was cooled down to rt, acidified with 1 N HCl solution and extracted with EtOAc (50 mL×3), the combined organic was dried over anhydrous Na₂SO₄, filtered and concentrated to leave crude intermediate 4 (400 mg, yield 100%) as white solid. LCMS (m/z): 346.1 [M+H]⁺.

### tert-butyl 2-(5-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethylcarbamate (Intermediate 5)

To a stirred solution of 5-benzyl-1-(2-(tert-butoxycarbonylamino)ethyl)-1H-pyrazole-4-carboxylic acid (138 mg, 0.4 mmol), 3-(dimethylamino)-N-(3-((4-hydroxypiperidin-4-yl)methyl)-4-oxo-3,4-dihydroquinazolin-7-yl)propanamide hydrochloride (164 mg, 0.4 mmol) and DIPEA (207 mg, 1.6 mmol) in DMF (6 mL) was added HATU (167 mg, 0.44 mmol). The mixture was stirred at rt for 1 hour. The mixture was diluted with EtOAc (100 mL), washed with H₂O (50 mL), dried over anhydrous Na₂SO₄, filtered and concentrated in vacuum. The residue was purified by flash column chromatograph (DCM/MeOH=10/1) to give intermediate 5 (100 mg, yield 35.7%) as white solid. LCMS: (m/z) 701.4 [M+H]⁺

### N-(3-((1-(1-(2-aminoethyl)-5-benzyl-1H-pyrazole-4-carbonyl)-4-hydroxypiperidin-4-yl)methyl)-4-oxo-3,4-dihydroquinazolin-7-yl)-3-(dimethylamino)propanamide hydrochloride (Intermediate 6)

To a solution of tert-butyl 2-(5-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethylcarbamate (100 mg, 0.143 mmol) in DCM (5 mL) was added HCl solution in 1,4-Dioxane (4 M, 0.8 mL, 3.2 mmol). The mixture was stirred at rt for 3 h and concentrated to leave crude product (101 mg, yield 100%) as HCl salt. LCMS: (m/z) 301.0 [M/2+H]⁺.

### N-(2-(5-benzyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethyl)-4-chloroquinoline-7-carboxamide (Compound 53)

To a stirred solution of N-(3-((1-(1-(2-aminoethyl)-5-benzyl-1H-pyrazole-4-carbonyl)-4-hydroxypiperidin-4-yl)methyl)-4-oxo-3,4-dihydroquinazolin-7-yl)-3-(dimethylamino)propanamide hydrochloride (101 mg, 0.158 mmol), 4-chloroquinoline-7-carboxylic acid (32.8 mg, 0.158 mmol) and DIEA (82 mg, 0.632 mmol) in DMF (5 mL) was added HATU (66 mg, 0.174 mmol). The mixture was stirred at rt for 1 hour, diluted with EtOAc (100 mL), washed with H₂O (50 mL), dried over anhydrous Na₂SO₄, filtered and concentrated in vacuum. The residue was purified by prep-HPLC (C18 column, CH₃CN/H₂O, containing 0.05% NH₄HCO₃) to give product (42.2 mg, yield 34%) as white solid. ¹H NMR (400 MHz, DMSO-d₆) δ (ppm) 10.52 (s, 1H), 9.07 (t, J=5.6 Hz, 1H), 8.92 (d, J=4.4 Hz, 1H), 8.53 (s, 1H), 8.27 (d, J=8.8 Hz, 1H), 8.21 (s, 1H), 8.15-8.04 (m, 3H), 7.84 (d, J=4.4 Hz, 1H), 7.64 (dd, J=1.6 Hz, 9.2 Hz, 1H), 7.60 (s, 1H), 7.29-7.10 (m, 5H), 4.99 (s, 1H), 4.26 (t, J=5.6 Hz, 2H), 4.14 (s, 2H), 3.99-3.57 (m, 6H), 3.29-2.98 (m, 2H), 2.61 (t, J=6.4 Hz, 2H), 2.54 (t, J=6.4 Hz, 2H), 2.20 (s, 6H), 2.08 (bs, 4H). LCMS: (m/z) 790.3 [M+H]⁺.

### Synthesis of Compounds 55

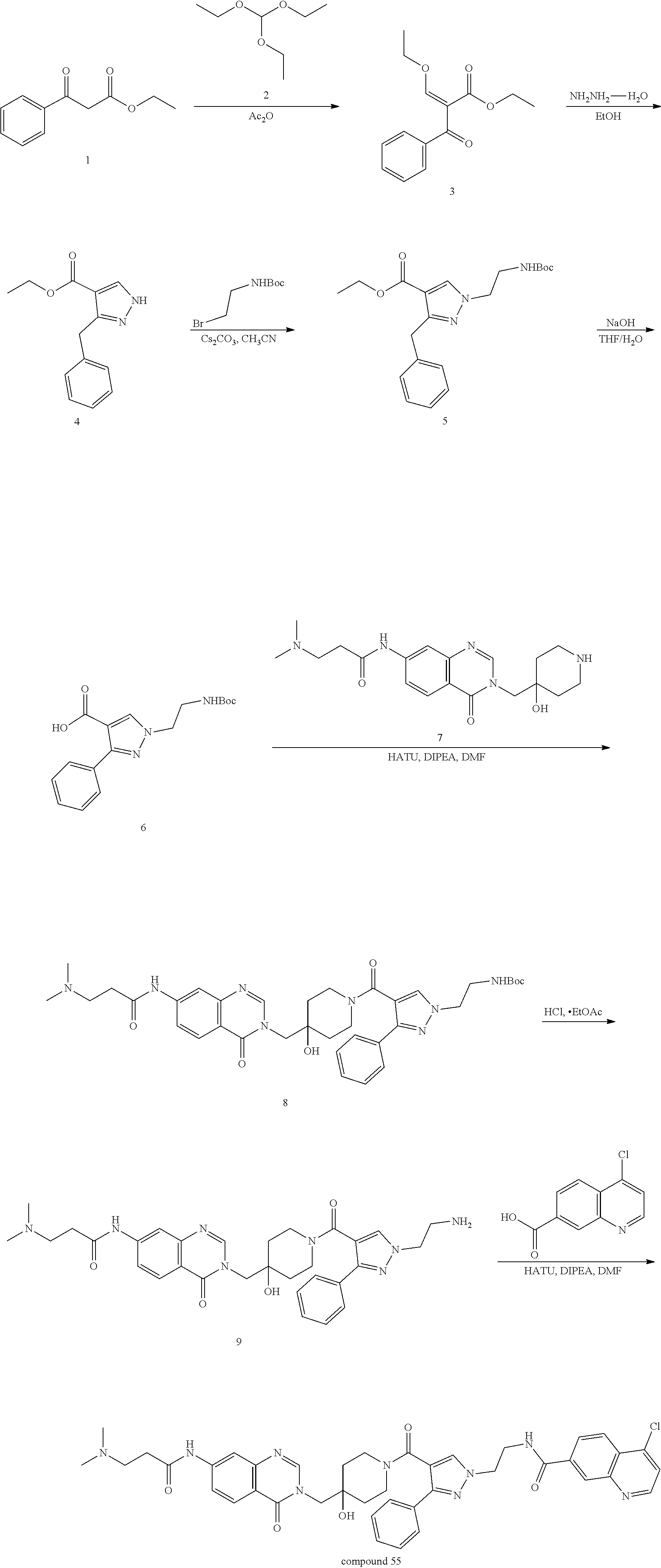

ethyl (Z)-2-benzoyl-3-ethoxyacrylate (Intermediate 3)

The mixture of ethyl 3-oxo-3-phenylpropanoate (1.0 g, 5.20 mmol) and tri-ethylorthoformate (1.6 g, 8.32 mmol) were heated to reflux for 30 minutes, and then acetic anhydride (1.6 g, 15.60 mmol) was added, the resulting mixture was heated to reflux for 12 h. The mixture was cooled down to rt, diluted with EtOAc (100 mL) and water (50 mL), the mixture was stirred at rt for 10 minutes to decompose excess triethylor-thoformate. The organic phase was separated, the aqueous layer was extracted with EtOAc (50 mL). The combined organic layers were washed with water (50 mL) and brine (50 mL), dried over anhydrous Na₂SO₄, filtered and concentrated in vacuo to leave crude intermediate 3 (1.5 g, crude) as yellow solid. LCMS (m/z): 249.1 [M+H]⁺

### ethyl 3-phenyl-1H-pyrazole-4-carboxylate (Intermediate 4)

A solution of hydrazine monohydrate (1.2 g, 2.21 mmol) in ethanol (1 mL) was added dropwise at 0° C. to a solution of ethyl (Z)-2-benzoyl-3-ethoxyacrylate (0.5 g, 2.01 mmol) in ethanol (5 mL). The reaction mixture was stirred at rt for 12 h and then filtered, the cake was washed with water and cold ethanol, and dried under vacuum to afford intermediate 4 (0.35 g, yield 80.4%) as yellow solid. LCMS (m/z): 217.1 [M+H]⁺

### Ethyl 1-(2-((tert-butoxycarbonyl)amino)ethyl)-3-phenyl-1H-pyrazole-4-carboxylate (Intermediate 5)

A mixture of ethyl 3-phenyl-1H-pyrazole-4-carboxylate (230.0 mg, 1.06 mmol), tert-butyl (2-bromoethyl)carbamate (238.4 mg, 1.06 mmol) and Cs₂CO₃ (345.4 mg, 1.06 mmol) in MeCN (10 mL) was stirred at 80° C. overnight, the mixture was cooled down to rt, diluted with H₂O (30 mL) and extracted with ethyl acetate (20 mL×3), the combined organic was washed with brine (50 mL), dried over anhydrous Na₂SO₄, concentrated and purified by flash column chromatography on silica gel (EA/PE: 5-20:1) to afford intermediate 5 (360 mg, yield 94.2%) as white solid. LCMS: (m/z) 360.2 [M+H]⁺

### 1-(2-((tert-butoxycarbonyl)amino)ethyl)-3-phenyl-1H-pyrazole-4-carboxylic acid (Intermediate 6)

A solution of NaOH (57.9 mg, 40.00 mmol) in H₂O (5 mL) was added to a solution of ethyl 1-(2-((tert-butoxycarbonyl)amino)ethyl)-3-phenyl-1H-pyrazole-4-carboxylate (260.0 mg, 0.72 mmol) in THF (5 mL), the mixture was stirred at 70° C. overnight. After cooled down to rt the mixture was adjusted to pH 1-2 with 1 N HCl solution and extracted with ethyl acetate (20 mL×3), the combined organic was washed with brine (50 mL), dried over anhydrous Na₂SO₄, filtered and concentrated to get 180 mg of crude product, which was purified by HPLC to get intermediate 6 (80 mg, yield 33%) as white solid. LCMS: (m/z) 332.2 [M+H]⁺.

### tert-butyl (2-(4-(4-((7-(3-(dimethylamino)propanamido)-4-oxoquinazolin-3(4H)-yl)methyl)-4-hydroxypiperidine-1-carbonyl)-3-phenyl-1H-pyrazol-1-yl)ethyl)carbamate (Intermediate 8)

To a mixture of 1-(2-((tert-butoxycarbonyl)amino)ethyl)-3-phenyl-1H-pyrazole-4-carboxylic acid (37.0 mg, 0.112 mmol), 3-(dimethylamino)-N-(3-((4-hydroxypiperidin-4-yl)methyl)-4-oxo-3,4-dihydroquinazolin-7-yl)propanamide (41.7 mg, 0.112 mmol) and DIPEA (46.3 mg, 0.336 mmol) in DMF (10 mL) was added HATU (46.8 mg, 0.123 mmol), the mixture was stirred at rt for 30 minutes, diluted with EtOAc (80 mL), washed with saturated NaHCO₃ solution (50 mL), dried over anhydrous Na₂SO₄, filtered and concentrated to leave the crude intermediate 8 (52.1 mg, yield 67.9%), which was used directly in the next step. LCMS: (m/z) 687.4 [M+H]⁺.

### N-(3-((1-(1-(2-aminoethyl)-3-phenyl-1H-pyrazole-4-carbonyl)-4-hydroxypiperidin-4-yl)methyl)-4-oxo-3,4-dihydroquinazolin-7-yl)-3-(dimethylamino)propanamide (Intermediate 9)

To the crude intermediate 8 (52.1 mg, 0.0759 mmol) was added HCl solution in EtOAc (1M, 5 mL), the mixture was stirred at rt overnight and concentrated under reduced pressure to leave crude intermediate 9 (45.3 mg, crude) as white solid. LCMS: (m/z) 587.3 [M+H]⁺.

### 4-chloro-N-(2-(4-(4-((7-(3-(dimethylamino)propanamido)-4-oxoquinazolin-3(4H)-yl)methyl)-4-hydroxypiperidine-1-carbonyl)-3-phenyl-1H-pyrazol-1-yl)ethyl)quinoline-7-carboxamide (Compound 55)

To a mixture of N-(3-((1-(1-(2-aminoethyl)-3-phenyl-1H-pyrazole-4-carbonyl)-4-hydroxypiperidin-4-yl)methyl)-4-oxo-3,4-dihydroquinazolin-7-yl)-3-(dimethylamino)propanamide (45.3 mg, 0.0772 mmol), 4-chloroquinoline-7-carboxylic acid (16.1 mg, 0.0772 mmol) and DIPEA (32.1 mg, 0.2316 mmol) in DMF (10 mL) was added HATU (32.3 mg, 0.0849 mmol), the mixture was stirred at rt for 30 minutes, diluted with EtOAc (80 mL), washed with saturated NaHCO₃ solution (50 mL), dried over anhydrous Na₂SO₄, concentrated and purified by prep-HPLC to get compound 55 (10.2 mg, yield 17.0%) as white solid. ¹H NMR (400 MHz, DMSO-d₆) δ (ppm) 10.62 (s, 1H), 9.05 (s, 1H), 8.88 (d, J=4.7 Hz, 1H), 8.56 (s, 1H), 8.23 (d, J=8.7 Hz, 1H), 8.16-8.10 (m, 2H), 8.05 (d, J=8.4 Hz, 2H), 7.96 (s, 1H), 7.82 (d, J=4.7 Hz, 1H), 7.62 (d, J=8.1 Hz, 1H), 7.54 (d, J=7.3 Hz, 2H), 7.31 (t, J=7.3 Hz, 2H), 7.25 (t, J=7.3 Hz, 1H), 4.83 (s, 1H), 4.40 (d, J=5.3 Hz, 2H), 4.18 (br, 1H), 4.0-3.75 (m, 5H), 3.05-1.95 (m, 2H), 2.81 (br, 2H), 2.66-2.55 (m, 2H), 2.35 (s, 6H), 1.42 (br, 2H), 1.20-0.95 (m, 2H). LCMS (m/z): 775.7 [M+H]⁺.

### Synthesis of Compound 56

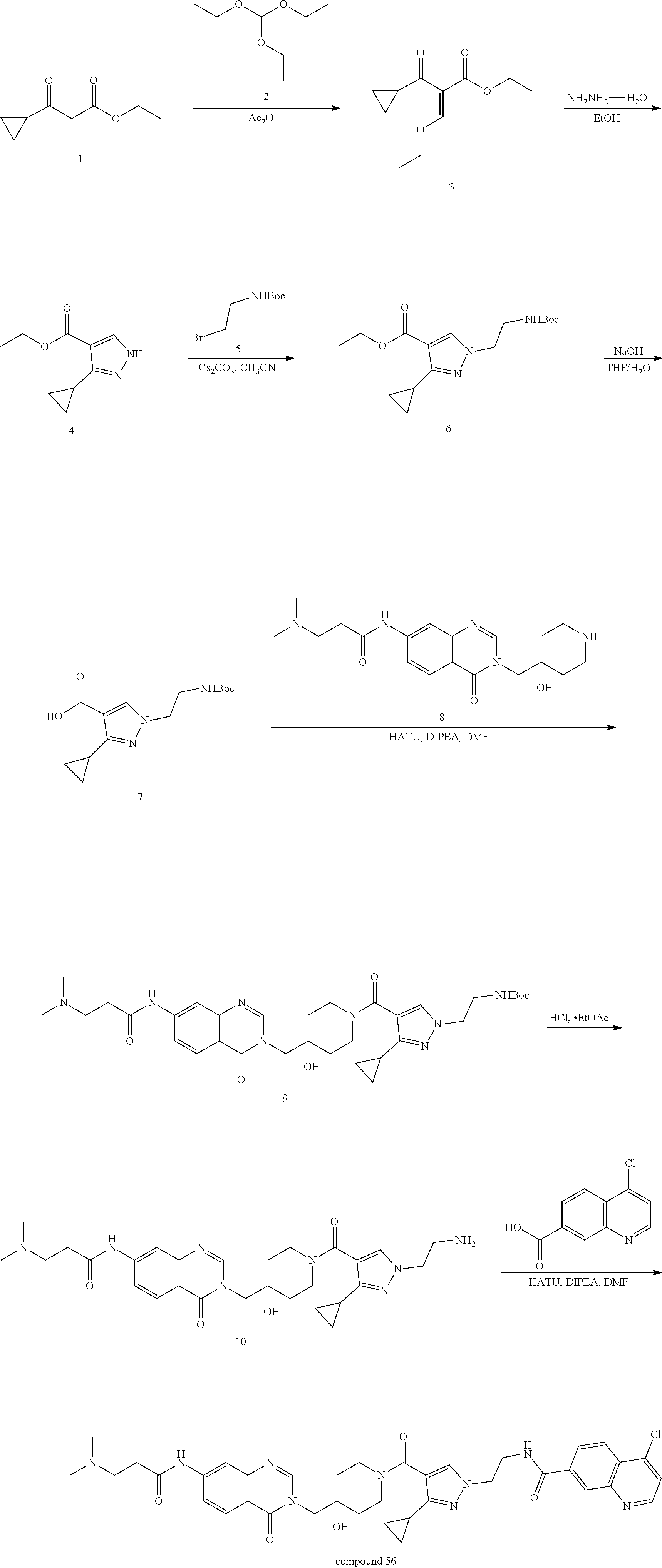

### ethyl (E)-2-(cyclopropanecarbonyl)-3-ethoxyacrylate (Intermediate 3)

A mixture of ethyl 3-cyclopropyl-3-oxopropanoate (1.0 g, 6.40 mmol) and tri-ethylorthoformate (1.1 g, 7.68 mmol) were heated to reflux for 30 minutes, and then acetic anhydride (1.96 g, 19.2 mmol) was added, the resulting mixture was heated to reflux for 12 h. The mixture was cooled down to rt, diluted with EtOAc (100 mL) and water (50 mL), the mixture was stirred at rt for 10 minutes to decompose excess triethylor-thoformate. The organic phase was separated, the aqueous layer was extracted with EtOAc (50 mL). The combined organic layers were washed with water (50 mL) and brine (50 mL), dried over anhydrous Na₂SO₄, filtered and concentrated in vacuo to leave crude intermediate 3 (1 g, yield 73.6%) as yellow solid. LCMS (m/z): 213.1 [M+H]⁺

### ethyl 3-cyclopropyl-1H-pyrazole-4-carboxylate (Intermediate 4)

A solution of hydrazine monohydrate (0.25 g, 5.18 mmol) in ethanol (10 mL) was added dropwise at 0° C. to a solution of ethyl (E)-2-(cyclopropanecarbonyl)-3-ethoxyacrylate (1.0 g, 4.71 mmol) in ethanol (10 mL). The reaction mixture was stirred at rt for 12 h, the resulting solid was collected, washed with water and cold ethanol, and dried under vacuum to afford crude product, which was further purified by flash column chromatography (PE:EA 20-10:1) to get intermediate 4 (560 mg, yield 66.0%) as yellow solid. LCMS (m/z): 181.1 [M+H]⁺

### ethyl 1-(2-((tert-butoxycarbonyl)amino)ethyl)-3-cyclopropyl-1H-pyrazole-4-carboxylate (Intermediate 6)

A mixture of ethyl 3-cyclopropyl-1H-pyrazole-4-carboxylate (560.0 mg, 3.11 mmol), tert-butyl (2-bromoethyl)carbamate (1044.6 mg, 4.66 mmol) and Cs₂CO₃ (1013.3 mg, 3.11 mmol) in CH₃CN (10 mL) was stirred at 80° C. overnight, the mixture was cooled down to rt, diluted with H₂O (30 mL) and extracted with ethyl acetate (20 mL×3), the combined organic was washed with brine (50 mL), dried over anhydrous Na₂SO₄, concentrated and purified by flash column chromatography on silica gel (PE/EA: 20:1˜10:1) to afford intermediate 6 (250 mg, yield 25.0%) as white solid. LCMS: (m/z) 324.2 [M+H]⁺

### 1-(2-((tert-butoxycarbonyl)amino)ethyl)-3-cyclopropyl-1H-pyrazole-4-carboxylic acid (Intermediate 7)

A solution of NaOH (49.5 mg, 1.24 mmol) in H₂O (10 mL) was added to a solution of ethyl 1-(2-((tert-butoxycarbonyl)amino)ethyl)-3-cyclopropyl-1H-pyrazole-4-carboxylate (200.0 mg, 0.62 mmol) in EtOH (5 mL), the mixture was stirred at 90° C. overnight. After cooled down to rt the mixture was adjusted to pH 1-2 with 1 N HCl solution and extracted with ethyl acetate (20 mL×3), the combined organic was washed with brine (50 mL), dried over anhydrous Na₂SO₄, filtered and concentrated to leave intermediate 7 (138 mg, yield 75.4%) as white solid. LCMS: (m/z) 296.2 [M+H]⁺.

### tert-butyl (2-(3-cyclopropyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethyl)carbamate (Intermediate 9)

To a mixture of 1-(2-((tert-butoxycarbonyl)amino)ethyl)-3-cyclopropyl-1H-pyrazole-4-carboxylic acid (59.1 mg, 0.20 mmol), 3-(dimethylamino)-N-(3-((4-hydroxypiperidin-4-yl)methyl)-4-oxo-3,4-dihydroquinazolin-7-yl)propanamide (74.1 mg, 0.20 mmol) and DIPEA (77.6 mg, 0.60 mmol) in DMF (10 mL) was added HATU (83.7 mg, 0.22 mmol), the mixture was stirred at rt for 30 minutes, diluted with EtOAc (80 mL), washed with saturated NaHCO₃ solution (50 mL), dried over anhydrous Na₂SO₄, filtered and concentrated to leave the crude compound, which was purified by flash column chromatography on silica gel (DCM/MeOH: 10:1) to afford intermediate 9 (50 mg, yield 38.5%) as white solid. LCMS: (m/z) 651.3 [M+H]⁺.

### N-(3-((1-(1-(2-aminoethyl)-3-cyclopropyl-1H-pyrazole-4-carbonyl)-4-hydroxypiperidin-4-yl)methyl)-4-oxo-3,4-dihydroquinazolin-7-yl)-3-(dimethylamino)propanamide (Intermediate 10)

To the crude compound 9 (50.0 mg, 0.077 mmol) was added HCl solution in EtOAc (1M, 5 mL), the mixture was stirred at rt overnight and concentrated under reduced pressure to leave crude intermediate 10 (50 mg, crude) as white solid. LCMS: (m/z) 551.3 [M+H]⁺.

### 4-chloro-N-(2-(3-cyclopropyl-4-(4-((7-(3-(dimethylamino)propanamido)-4-oxoquinazolin-3(4H)-yl)methyl)-4-hydroxypiperidine-1-carbonyl)-1H-pyrazol-1-yl)ethyl)quinoline-7-carboxamide (Compound 56)

To a mixture of N-(3-((1-(1-(2-aminoethyl)-3-cyclopropyl-1H-pyrazole-4-carbonyl)-4-hydroxypiperidin-4-yl)methyl)-4-oxo-3,4-dihydroquinazolin-7-yl)-3-(dimethylamino)propanamide (50.0 mg, 0.10 mmol), 4-chloroquinoline-7-carboxylic acid (20.8 mg, 0.10 mmol) and DIPEA (38.8 mg, 0.30 mmol) in DMF (10 mL) was added HATU (41.8 mg, 0.11 mmol), the mixture was stirred at rt for 30 minutes, diluted with EtOAc (80 mL), washed with saturated NaHCO₃ solution (50 mL), dried over anhydrous Na₂SO₄, concentrated and purified by prep-HPLC to afford compound 56 (1.3 mg, yield 1.93%) as white solid. ¹H NMR (400 MHz, DMSO-d₆) δ (ppm) 10.49 (s, 1H), 8.95 (s, 1H), 8.88 (d, J=4.6 Hz, 1H), 8.52 (s, 1H), 8.27-8.16 (m, 2H), 8.08 (dd, J=19.2, 8.7 Hz, 3H), 7.86-7.76 (m, 2H), 7.62 (d, J=8.7 Hz, 1H), 4.96 (s, 1H), 4.23 (t, J=5.7 Hz, 2H), 3.94 (s, 2H), 3.75-3.6 (m, 2H), 3.25-3.10 (m, 4H), 2.58 (d, J=5.9 Hz, 2H), 2.54 (s, 1H), 2.18 (s, 6H), 1.99-1.88 (m, 2H), 1.55-1.45 (m, 2H), 1.45-1.30 (m, 2H), 0.78 (d, J=8.0 Hz, 2H), 0.72 (d, J=2.7 Hz, 2H). LCMS (m/z): 740.3 [M+H]⁺.

### Synthesis of Compounds 58 and 59

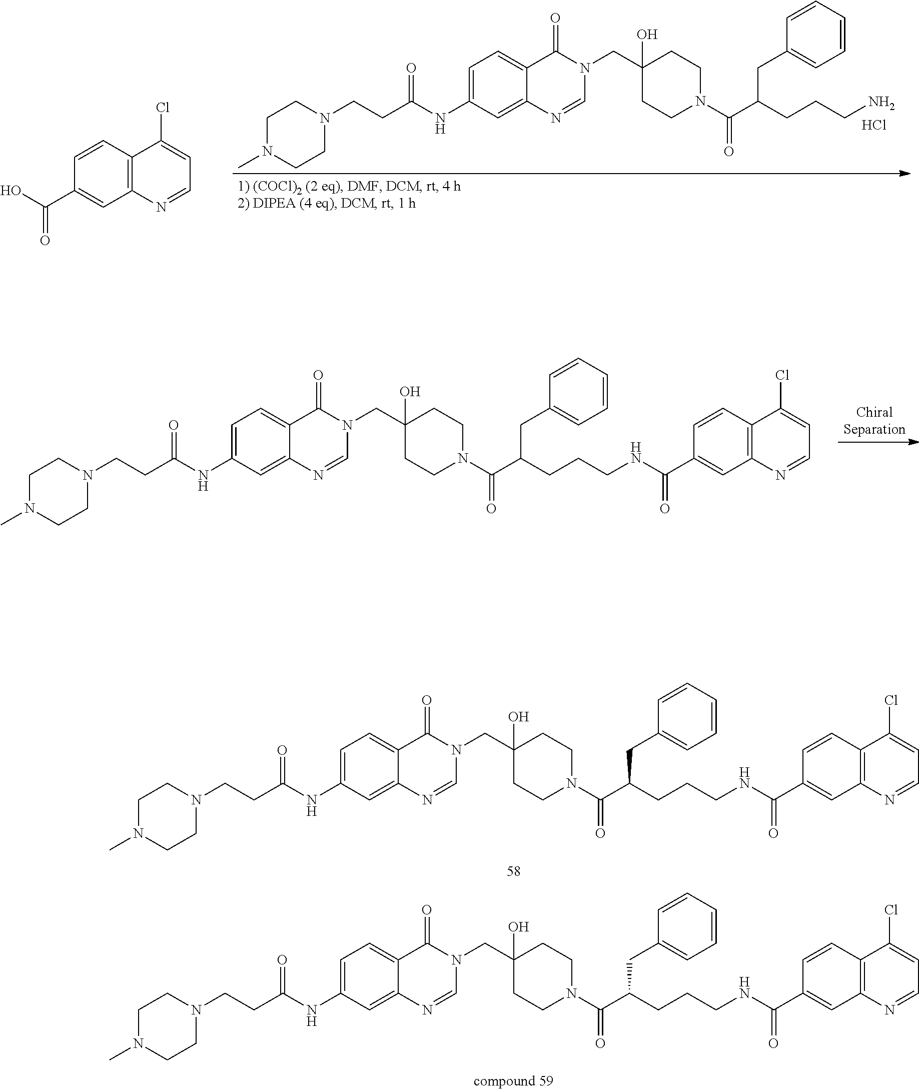 [SMILES: O=C(O)c1ccc2c(Cl)ccnc2c1 ; CN1CCN(CCC(=O)Nc2ccc3c(=O)n(CC4(O)CCN(C(=O)C(CCCN)Cc5ccccc5)CC4)cnc3c2)CC1 ; Cl ; CN1CCN(CCC(=O)Nc2ccc3c(=O)n(CC4(O)CCN(C(=O)C(CCCNC(=O)c5ccc6c(Cl)ccnc6c5)Cc5ccccc5)CC4)cnc3c2)CC1 ; CN1CCN(CCC(=O)Nc2ccc3c(=O)n(CC4(O)CCN(C(=O)[C@H](CCCNC(=O)c5ccc6c(Cl)ccnc6c5)Cc5ccccc5)CC4)cnc3c2)CC1 ; CN1CCN(CCC(=O)Nc2ccc3c(=O)n(CC4(O)CCN(C(=O)[C@@H](CCCNC(=O)c5ccc6c(Cl)ccnc6c5)Cc5ccccc5)CC4)cnc3c2)CC1]

### N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide

To a mixture of 4-chloroquinoline-7-carboxylic acid (83 mg, 0.4 mmol) in dry DCM (10 mL) was added DMF (2 drops), followed by addition of (COCl)₂ (101 mg, 0.8 mmol). The mixture was stirred at rt for 4 hours and concentrated in vacuum, the residue was re-dissolved in DCM (10 mL) and added dropwise to a mixture of N-(3-((1-(5-amino-2-benzylpentanoyl)-4-hydroxypiperidin-4-yl)methyl)-4-oxo-3,4-dihydroquinazolin-7-yl)-3-(4-methylpiperazin-1-yl)propanamide hydrochloride (209 mg, 0.32 mmol) and DIPEA (155 mg, 1.2 mmol) in DCM (10 mL) at 0° C. The mixture was stirred at rt for 1 hour, concentrated and purified by prep-HPLC (C18 column, CH₃CN/H₂O, containing 0.05% NH₄HCO₃) to give N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (75.7 mg, yield 29%) as a white solid. ¹H NMR (400 MHz, DMSO-d₆) δ (ppm) 10.52 (d, J=4.0 Hz, 1H), 8.95-8.85 (m, 2H), 8.60 (dd, J=1.2 Hz, 10.8 Hz, 1H), 8.28 (dd, J=5.6 Hz, 10.8 Hz, 1H), 8.21-7.98 (m, 5H), 7.85 (d, J=4.8 Hz, 1H), 7.65-7.58 (m, 1H), 7.27-7.07 (m, 5H), 4.84 (s, 1H), 4.20-3.98 (m, 1H), 3.97-3.56 (m, 3H), 3.34-3.08 (m, 4H), 2.91-2.60 (m, 6H), 2.58-2.52 (m, 2H), 2.48-2.21 (m, 7H), 2.15 (s, 3H), 1.72-1.02 (m, 8H). LCMS (m/z): 807.4 [M+H]⁺.

### (R)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (Compound 58)

### (S)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (Compound 59)

N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (20 mg, 0.025 mmol) was separated by Chiral HPLC (Instrument: Gilson-281, Column: IC, 20*250, 10 um; Mobile Phase: MEOH (0.2% Methanol Ammonia):CAN (0.2% Methanol Ammonia)=50:50; Flow Rate: 50 mL/min; Run time: per injection: 30 min.) to give (R)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (8.8 mg) as solid and (S)—N-(4-benzyl-5-(4-hydroxy-4-((7-(3-(4-methylpiperazin-1-yl)propanamido)-4-oxoquinazolin-3(4H)-yl)methyl)piperidin-1-yl)-5-oxopentyl)-4-chloroquinoline-7-carboxamide (9.1 mg) as solid. LCMS (m/z): compound 58, 807.4 [M+H]⁺; compound 59, 807.4 [M+H]⁺.

### Synthesis of Compound 60

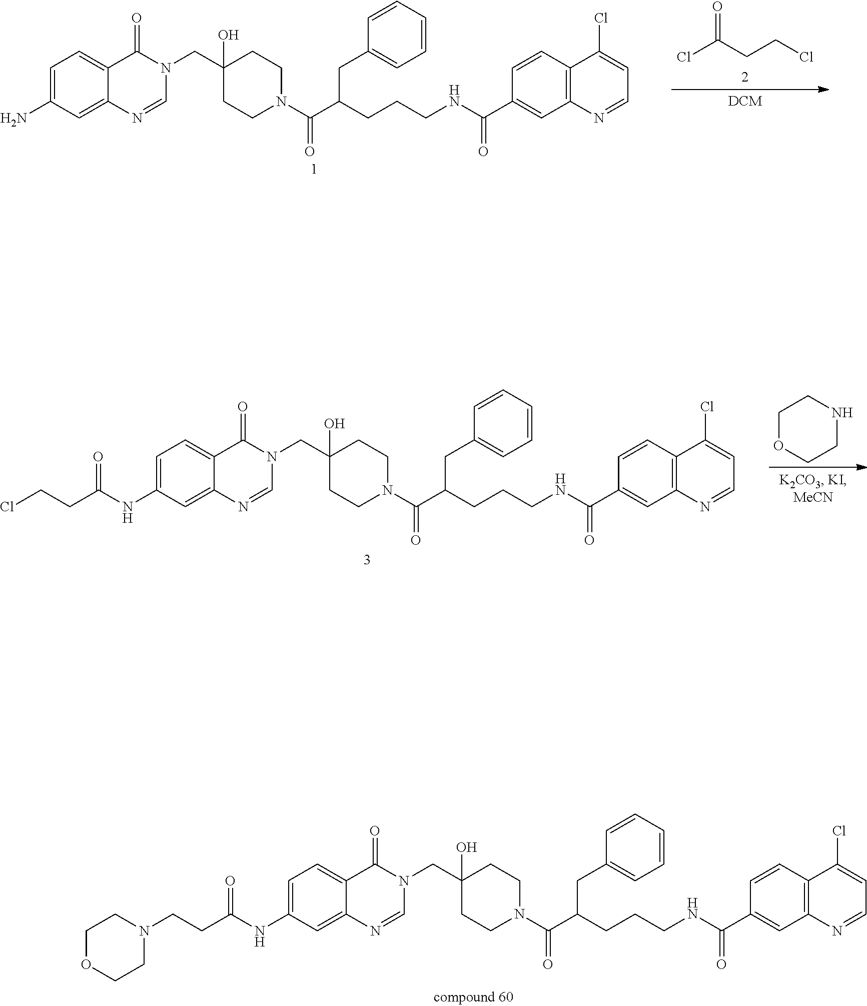

### N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (Intermediate 3)

To a mixture of N-(5-(4-((7-amino-4-oxoquinazolin-3(4H)-yl)methyl)-4-hydroxypiperidin-1-yl)-4-benzyl-5-oxopentyl)-4-chloroquinoline-7-carboxamide (400.0 mg, 0.6124 mmol) in dry DCM (10 mL) was added dropwised 3-chloropropanoyl chloride (233.2 mg, 1.8372 mmol), the mixture was stirred at rt for 1 h, and then MTBE (10 mL) was added, the resulting suspension was filtrated, the cake was dried under vacuum to afford intermediate 3 (350 mg, yield 76.9%) as white solid. LCMS (m/z): 743.2 [M+H]⁺

### N-(4-benzyl-5-(4-hydroxy-4-((7-(3-morpholinopropanamido)-4-oxoquinazolin-3(4H)-yl)methyl)piperidin-1-yl)-5-oxopentyl)-4-chloroquinoline-7-carboxamide (Compound 60)

A mixture of N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (60.0 mg, 0.0807 mmol), morpholine (21.1 mg, 0.242 mmol), K₂CO₃ (55.7 mg, 0.403 mmol) and KI (77.0 mg, 0.403 mmol) in MeCN (5 ml) was heated at 120° C. under microwave irradiation for 3 hours, the mixture was concentrated in vacuum, the residue was diluted with H₂O (20 mL) and extracted with ethyl acetate (20 mL*3), the combined organic was washed with brine (50 mL), dried over anhydrous Na₂SO₄, concentrated and purified by prep-HPLC to obtain compound 60 (19.5 mg, yield 30.5%) as white solid. ¹H NMR (400 MHz, DMSO-d₆) δ (ppm) 10.66 (d, J=4.8 Hz, 1H), 9.78 (s, 1H), 8.98-8.84 (m, 2H), 8.60 (d, J=9.7 Hz, 1H), 8.28 (dd, J=8.7 Hz, 5.4 Hz, 1H), 8.19-8.12 (m, 2H), 8.08-8.03 (m, 1H), 7.86 (d, J=4.7 Hz, 1H), 7.70-7.57 (m, 1H), 7.25-7.07 (m, 5H), 4.83 (s, 1H), 4.13 (d, J=12.1 Hz, 1H), 3.99 (s, 2H), 3.94-3.87 (m, 1H), 3.82 (d, J=13.8 Hz, 1H), 3.68-3.60 (m, 5H), 3.30 (s, 2H), 3.14 (s, 4H), 2.94 (t, J=6.8 Hz, 2H), 2.88-2.81 (m, 1H), 2.80-2.61 (m, 4H), 1.65 (s, 1H), 1.50 (s, 3H), 1.28-1.23 (m, 2H), 4.96 (s, 1H), 1.15-1.0 (m, 2H). LCMS (m/z): 794.3 [M+H]⁺.

### Synthesis of Compound 61

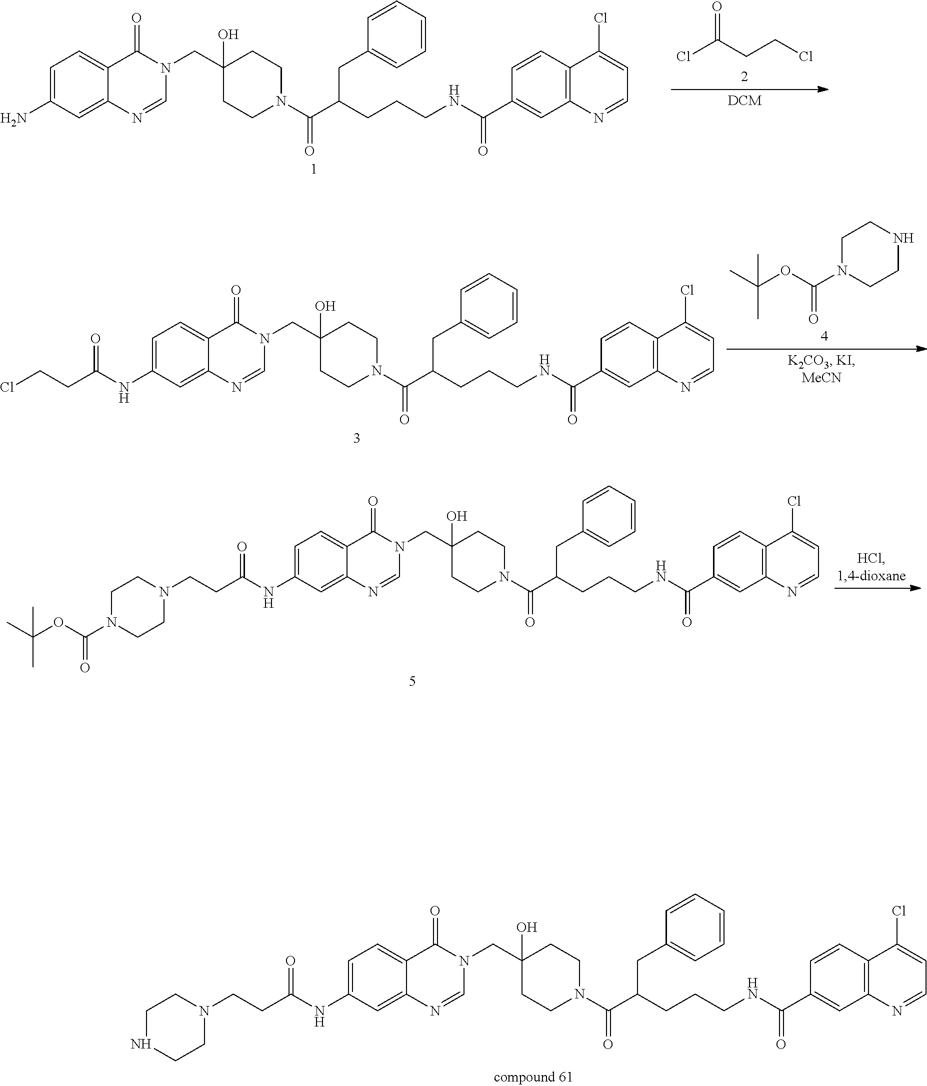 [SMILES: Nc1ccc2c(=O)n(CC3(O)CCN(C(=O)C(CCCNC(=O)c4ccc5c(Cl)ccnc5c4)Cc4ccccc4)CC3)cnc2c1 ; O=C(Cl)CCCl ; O=C(CCCl)Nc1ccc2c(=O)n(CC3(O)CCN(C(=O)C(CCCNC(=O)c4ccc5c(Cl)ccnc5c4)Cc4ccccc4)CC3)cnc2c1 ; CC(C)(C)OC(=O)N1CCNCC1 ; CC(C)(C)OC(=O)N1CCN(CCC(=O)Nc2ccc3c(=O)n(CC4(O)CCN(C(=O)C(CCCNC(=O)c5ccc6c(Cl)ccnc6c5)Cc5ccccc5)CC4)cnc3c2)CC1 ; O=C(CCN1CCNCC1)Nc1ccc2c(=O)n(CC3(O)CCN(C(=O)C(CCCNC(=O)c4ccc5c(Cl)ccnc5c4)Cc4ccccc4)CC3)cnc2c1]

### N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (Intermediate 3)

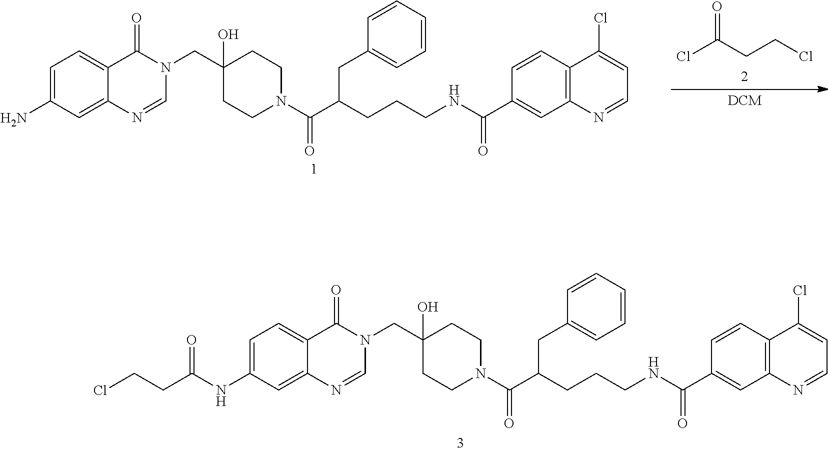 [SMILES: Nc1ccc2c(=O)n(CC3(O)CCN(C(=O)C(CCCNC(=O)c4ccc5c(Cl)ccnc5c4)Cc4ccccc4)CC3)cnc2c1 ; O=C(Cl)CCCl ; O=C(CCCl)Nc1ccc2c(=O)n(CC3(O)CCN(C(=O)C(CCCNC(=O)c4ccc5c(Cl)ccnc5c4)Cc4ccccc4)CC3)cnc2c1]

To a mixture of N-(5-(4-((7-amino-4-oxoquinazolin-3(4H)-yl)methyl)-4-hydroxypiperidin-1-yl)-4-benzyl-5-oxopentyl)-4-chloroquinoline-7-carboxamide (400 mg, 0.612 mmol) in dry DCM (10 mL) was added dropwise 3-chloropropanoyl chloride (233 mg, 1.837 mmol), the mixture was stirred at rt for 1 h, and then MTBE (10 mL) was added, the resulting suspension was filtrated, the cake was dried under vacuum to afford intermediate 3 (350 mg, yield 76.9%) as white solid. LCMS (m/z): 743.2 [M+H]⁺

### tert-butyl 4-(3-((3-((1-(2-benzyl-5-(4-chloroquinoline-7-carboxamido)pentanoyl)-4-hydroxypiperidin-4-yl)methyl)-4-oxo-3,4-dihydroquinazolin-7-yl)amino)-3-oxopropyl)piperazine-1-carboxylate (Intermediate 5)

A mixture of N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (60.0 mg, 0.081 mmol), tert-butyl piperazine-1-carboxylate (45.1 mg, 0.242 mmol), K₂CO₃ (55.7 mg, 0.403 mmol) and KI (66.9 mg, 0.403 mmol) in MeCN (5 ml) was heated at 120° C. under microwave irradiation for 3 hours, the mixture was concentrated in vacuum, the residue was diluted with H₂O (20 mL) and extracted with ethyl acetate (20 mL*3), the combined organic was washed with brine (50 mL), dried over anhydrous Na₂SO₄, concentrated and purified by prep-HPLC to afford intermediate 5 (30 mg, Yield 41.6%) as white solid. LCMS (m/z): 893.4 [M+H]⁺.

### N-(4-benzyl-5-(4-hydroxy-4-((4-oxo-7-(3-(piperazin-1-yl)propanamido)quinazolin-3(4H)-yl)methyl)piperidin-1-yl)-5-oxopentyl)-4-chloroquinoline-7-carboxamide (Compound 61)

To a mixture of tert-butyl 4-(3-((3-((1-(2-benzyl-5-(4-chloroquinoline-7-carboxamido)pentanoyl)-4-hydroxypiperidin-4-yl)methyl)-4-oxo-3,4-dihydroquinazolin-7-yl)amino)-3-oxopropyl)piperazine-1-carboxylate (30 mg, 0.03 mmol) in 1,4-dioxane (5 mL) was added HCl solution in 1,4-dioxane (5 mL), the mixture was stirred at rt for 5 hours, concentrated and purified by prep-HPLC to afford compound 61 (5.2 mg, yield 19.5%) as white solid. ¹H NMR (400 MHz, DMSO-d₆) δ (ppm) 10.59 (d, J=4.8 Hz, 1H), 8.93 (dd, J=4.7, 1.5 Hz, 1H), 8.89 (dt, J=11.2, 5.7 Hz, 1H), 8.63-8.55 (m, 1H), 8.28 (dd, J=8.7, 5.3 Hz, 1H), 8.16 (ddd, J=26.6, 17.5, 5.5 Hz, 3H), 8.01 (dd, J=6.6, 1.8 Hz, 1H), 7.85 (d, J=4.7 Hz, 1H), 7.65-7.58 (m, 1H), 7.17 (dtd, J=23.6, 15.5, 7.4 Hz, 6H), 4.85 (s, 1H), 4.17-3.97 (m, 1H), 3.94-3.79 (m, 1H), 3.63 (d, J=13.7 Hz, 2H), 3.15 (d, J=9.7 Hz, 2H), 2.88-2.82 (m, 1H), 2.79-2.73 (m, 2H), 2.69 (s, 4H), 2.63-2.58 (m, 4H), 2.56-2.53 (m, 2H), 2.36 (s, 4H), 1.75-1.60 (m, 1H), 1.50 (s, 3H), 1.40-1.25 (m, 2H), 1.20-1.0 (m, 2H). LCMS (m/z): 793.3 [M+H]⁺.

### Synthesis of Compound 63

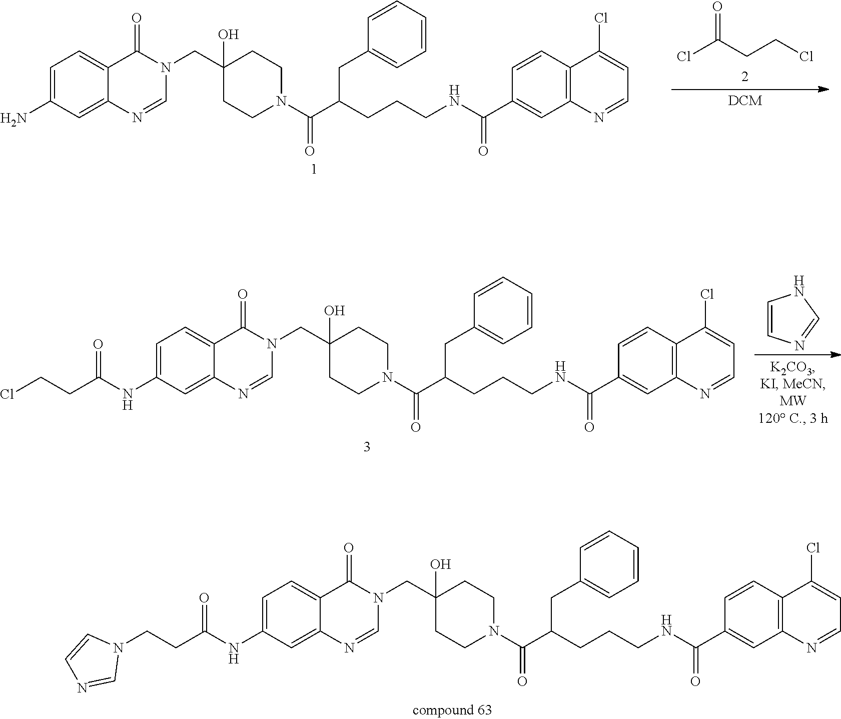

### N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (Intermediate 3)

To a mixture of N-(5-(4-((7-amino-4-oxoquinazolin-3(4H)-yl)methyl)-4-hydroxypiperidin-1-yl)-4-benzyl-5-oxopentyl)-4-chloroquinoline-7-carboxamide (400 mg, 0.612 mmol) in Dry DCM (10 mL) was added dropwise 3-chloropropanoyl chloride (233.2 mg, 1.837 mmol), the mixture was stirred at rt for 1 h, and then MTBE (10 mL) was added, the resulting suspension was filtrated, the cake was dried under vacuum to afford intermediate 3 (350 mg, yield 76.9%) as white solid. LCMS (m/z): 743.2 [M+H]⁺

### N-(5-(4-((7-(3-(1H-imidazol-1-yl)propanamido)-4-oxoquinazolin-3(4H)-yl)methyl)-4-hydroxypiperidin-1-yl)-4-benzyl-5-oxopentyl)-4-chloroquinoline-7-carboxamide (Compound 63)

A mixture of N-(4-benzyl-5-(4-((7-(3-chloropropanamido)-4-oxoquinazolin-3(4H)-yl)methyl)-4-hydroxypiperidin-1-yl)-5-oxopentyl)-4-chloroquinoline-7-carboxamide (60.0 mg, 0.081 mmol), 1H-imidazole (45.1 mg, 0.242 mmol), K₂CO₃ (55.7 mg, 0.403 mmol) and KI (66.9 mg, 0.403 mmol) in MeCN (5 mL) was heated at 120° C. under microwave irradiation for 3 hours, the mixture was concentrated in vacuum, the residue was diluted with H₂O (20 mL) and extracted with ethyl acetate (20 mL*3), the combined organic was washed with brine (50 mL), dried over anhydrous Na₂SO₄, concentrated and purified by prep-HPLC to afford compound 63 (2.0 mg, Yield 4.0%) as white solid. ¹H NMR (400 MHz, DMSO-d₆) δ (ppm) 10.60 (d, J=4.7 Hz, 1H), 9.06 (s, 1H), 8.97-8.83 (m, 2H), 8.60 (d, J=9.7 Hz, 1H), 8.28 (dd, J=8.6, 4.8 Hz, 1H), 8.20-7.97 (m, 4H), 7.86 (d, J=4.7 Hz, 1H), 7.76 (s, 1H), 7.60 (d, J=17.3 Hz, 2H), 7.26-7.08 (m, 5H), 4.85 (s, 1H), 4.51 (t, J=5.8 Hz, 2H), 4.10-4.05 (m, 1H), 3.90-3.75 (m, 1H), 3.63 (d, J=13.6 Hz, 2H), 3.14 (s, 2H), 3.07 (s, 2H), 2.84 (d, J=10.9 Hz, 1H), 2.75-2.60 (m, 2H), 2.69-2.52 (m, 2H), 1.65 (s, 1H), 1.50 (s, 3H), 1.35-1.25 (m, 2H), 1.18-1.00 (m, 2H). LCMS (m/z): 775.3 [M+H]⁺.

### Synthesis of Compound 64

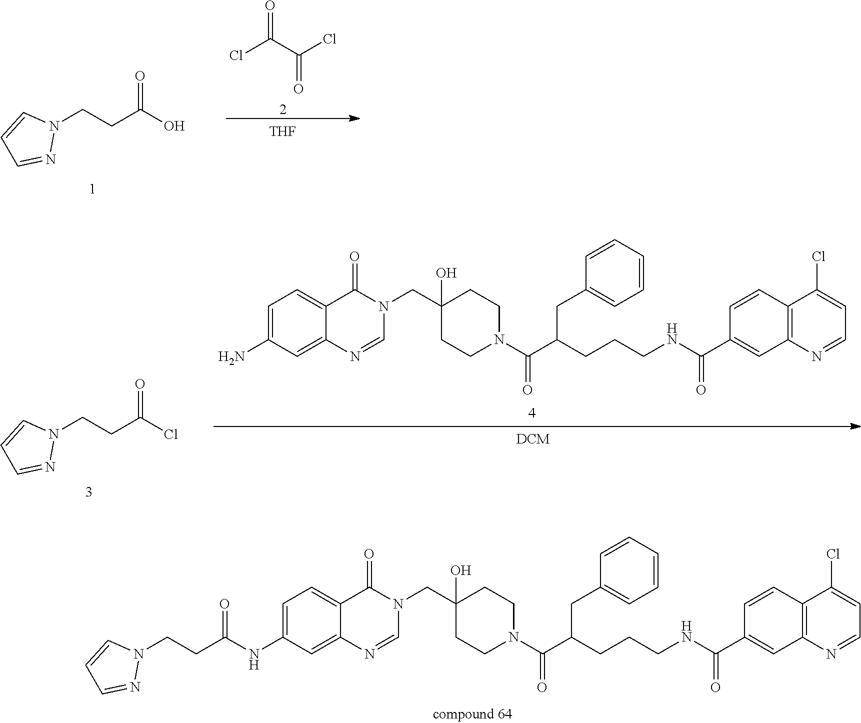

### 3-(1H-pyrazol-1-yl)propanoyl chloride (Intermediate 3)

To an ice-cooled solution of 3-(1H-pyrazol-1-yl)propanoic acid (14.0 mg, 0.10 mmol) in THF (5.0 mL) was added oxalyl dichloride (63.5 mg, 0.50 mmol), the mixture was stirred at 0-5° C. for 10 minutes and then stirred at room temperature for another 1 hours. The mixture was concentrated to leave crude intermediate 3 (18 mg, crude) as red solid. LCMS (m/z): 155.1 [M+H]⁺

### N-(5-(4-((7-(3-(JH-pyrazol-1-yl)propanamido)-4-oxoquinazolin-3(4H)-yl)methyl)-4-hydroxypiperidin-1-yl)-4-benzyl-5-oxopentyl)-4-chloroquinoline-7-carboxamide (Compound 64)

The solution of the above crude 3-(1H-pyrazol-1-yl)propanoyl chloride (18.0 mg, 0.113 mmol) in dry DCM (2 mL) was added dropwise to a solution of N-(5-(4-((7-amino-4-oxoquinazolin-3(4H)-yl)methyl)-4-hydroxypiperidin-1-yl)-4-benzyl-5-oxopentyl)-4-chloroquinoline-7-carboxamide (37.1 mg, 0.0567 mmol) in DCM (5 mL) at 0° C., the mixture was stirred at room temperature for 2 h, diluted with H₂O (10 mL) and extracted with DCM (20 mL×3), the combined organic was washed with brine (50 mL), dried over anhydrous Na₂SO₄, concentrated and purified by prep-HPLC to get compound 64 (3.1 mg, Yield 7.0%) as white solid. ¹H NMR (400 MHz, DMSO-d₆) δ (ppm) 10.46 (d, J=4.7 Hz, 1H), 8.93 (d, J=4.7 Hz, 1H), 8.88 (dt, J=11.0, 5.6 Hz, 1H), 8.60 (d, J=10.5 Hz, 1H), 8.28 (dd, J=8.7, 5.3 Hz, 1H), 8.21-8.04 (m, 3H), 8.03-7.99 (m, 1H), 7.85 (d, J=4.7 Hz, 1H), 7.71 (d, J=2.0 Hz, 1H), 7.64-7.55 (m, 1H), 7.44 (d, J=1.4 Hz, 1H), 7.23 (dd, J=15.7, 8.2 Hz, 2H), 7.18-7.07 (m, 3H), 6.21 (t, J=2.0 Hz, 1H), 4.84 (s, 1H), 4.45 (t, J=6.7 Hz, 2H), 4.20-4.0 (in, 1H), 3.94-3.79 (in, 1H), 3.63 (d, J 13.6 Hz, 2H), 3.35-3.25 (m, 2H), 3.20-3.10 (m, 2H), 2.97 (J=6.7 Hz, 2H), 2.85 (t, J=10.9 Hz, 11H), 2.76-2.65 (m, 2H), 1.75-1.60 (m, 11H), 1.55-1.0 (in, 7H). LCMS (m/z): 775.3 [M+H]⁺.

### Example 2: In Vitro Assays Using Exemplar Compounds of the Disclosure

Enzymatic Assays with Exemplary USP7 Irreversible Inhibitors

Tables 1 and 2: USP7 activity of exemplary compounds in USP7 assay. ++++ indicates an IC₅₀ of less than about 20 nM, +++ indicates an IC₅₀ from about 20 nM to about 100 nM, ++ indicates an IC₅₀ from about 100 nM to about 1 μM, and + indicates an IC₅₀ greater than 1 μM; mouse liver microsomal stability of exemplary compounds; plasma stability of exemplary compounds. ND refers to not disclosed.

TABLE 1

| | IC₅₀, | MLM t_{1/2}, | Plasma t_{1/2}, | | IC₅₀, | MLM t_{1/2}, | Plasma t_{1/2}, |
|---|---|---|---|---|---|---|---|
| Compound | μM | min | min | Compound | μM | min | min |
| 4 | + | ND | ND | 58 | + | ND | ND |
| 5 | ++ | 18 | 320 | 59 | +++ | ND | 78.9 |
| 6 | +++ | 1.0 | ND | 60 | +++ | 2.71 | ND |
| 7 | ++++ | 4.3 | ND | 61 | +++ | 28.41 | 193.37 |
| 8 | +++ | ND | ND | 62 | +++ | 12.28 | 193.72 |
| 9 | +++ | ND | ND | 63 | ++ | 4.16 | ND |
| 10 | +++ | 1.5 | ND | 64 | ++ | ND | ND |
| 11 | +++ | 12.1 | ND | 65 | ++ | 12.8 | ND |
| 12 | + | ND | ND | 66 | + | ND | ND |
| 13 | +++ | ND | 91.9 | 67 | + | ND | ND |
| 14 | ++ | ND | ND | 68 | + | ND | ND |
| 15 | ++ | ND | ND | 69 | + | 4.5 | ND |
| 16 | ++ | ND | ND | 70 | +++ | 10.3 | ND |
| 17 | +++ | ND | ND | 71 | ++ | 54.6 | ND |
| 18 | +++ | 21.2 | ND | 72 | ++ | 29.5 | ND |
| 19 | + | ND | ND | 73 | +++ | 46.84 | 191.17 |
| 20 | +++ | 25.8 | 226.0 | 74 | + | NL | ND |
| 21 | + | 18.5 | ND | 75 | ++++ | 69.41 | 67.42 |
| 22 | +++ | 18.3 | 134.9 | 76 | ++ | ND | ND |
| 23 | +++ | 49.3 | 10.5 | 78 | ++++ | 14.55 | 77.26 |
| 24 | + | ND | 23.0 | 79 | +++ | 10.48 | 234.31 |
| 25 | + | ND | ND | 80 | + | 5.41 | ND |
| 26 | + | ND | ND | 81 | +++ | 11.85 | ND |
| 27 | + | 20.0 | ND | 82 | ++ | ND | ND |
| 28 | + | ND | ND | 83 | + | ND | ND |
| 29 | +++ | 16.7 | ND | 87 | ++++ | 23.17 | 60.79 |
| 30 | +++ | 15.7 | 6706.0 | 88 | + | ND | ND |
| 31 | ++ | 2.9 | ND | 89 | +++ | 3.34 | ND |
| 32 | +++ | 2.5 | ND | 90 | +++ | ND | ND |
| 33 | ++++ | 2.1 | ND | 91 | +++ | 2.97 | 335.22 |
| 34 | + | ND | ND | 92 | +++ | ND | ND |
| 35 | + | ND | ND | 93 | ++++ | 14.68 | 10.31 |
| 36 | + | ND | ND | 94 | ++ | 30.78 | 1.31 |
| 37 | + | ND | ND | 98 | ++ | ND | ND |
| 38 | + | ND | ND | 99 | + | ND | ND |
| 39 | + | ND | ND | 100 | + | ND | ND |
| 40 | +++ | 7.4 | ND | 101 | + | ND | ND |
| 41 | ++++ | ND | ND | 102 | ++ | 26.91 | 4.25 |
| 42 | ++ | ND | ND | 103 | ++ | ND | ND |
| 43 | +++ | 8.3 | 185.7 | 104 | ++ | ND | ND |
| 44 | + | ND | ND | | | | |
| 45 | + | ND | ND | 106 | ++ | 38.95 | 6.8 |
| | | | | | (1 h) | | |
| | | | | | ++++ | | |
| | | | | | (6 h) | | |
| | | | | | ++++ | | |
| | | | | | (16 h) | | |
| 46 | + | ND | ND | 107 | +++ | ND | ND |
| 47 | +++ | 12.4 | | 108 | ++++ | 14 | ∞* |
| 48 | + | ND | ND | 109 | ++++ | 22.41 | 101.85 |
| 49 | ++++ | 13.5 | ND | 110 | ++ | 2.21 | ND |
| 50 | + | ND | ND | 112 | ++ | 45.26 | 305.08 |
| 51 | +++ | ND | ND | 114 | ++++ | 4.13 | 702.27 |
| 52 | ++ | 15.1 | ND | 116 | ++++ | 27.08 | ∞* |
| 53 | ++ | ND | ND | 117 | +++ | 20.28 | ∞* |
| 54 | +++ | 36.5 | ND | 118 | ++ | ND | ND |
| 55 | + | 35.6 | ND | 119 | ++++ | 3.04 | ND |
| 77 | +++ | 1.73 | 160.8 | 120 | ++++ | 5.89 | ND |
| 130 | ++++ | ND | ND | 121 | ++++ | 6 | ND |
| 131 | ++ | ND | ND | 122 | +++ | 3091 | 226.19 |
| 56 | + | 95.9 | ND | 123 | ++++ | 24.26 | 177.72 |
| 57 | + | ND | ND | 136 | ++++ | ND | ND |
| 132 | +++ | 6.96 | ∞* | 138 | +++ | ND | ND |
| 135 | +++ | ND | ND | 136 | ++++ | ND | ND |
| 144 | +++ | ND | ND | | | | |

*If percent remaining at 60 min was higher than 50%, then t_{1/2} was calculated using extrapolation method; if percentage remaining at 60 min was near 100% and the value of t_{1/2} <0 by Excel default, then t_{1/2} were reported as “∞”.

TABLE 2

| | | MLM | Plasma |
|---|---|---|---|
| | IC₅₀, | t_{1/2}, | t_{1/2}, |
| Compound | μM | Min | min |
| 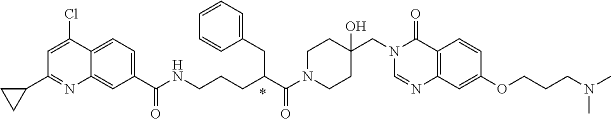 [SMILES: CN(C)CCCOc1ccc2c(=O)n(CC3(O)CCN(C(=O)C(CCCNC(=O)c4ccc5c(Cl)cc(C6CC6)nc5c4)Cc4ccccc4)CC3)cnc2c1] | ND | ND | ND |
| (absolute stereochemistry not verified; opposite stereochemistry to 116) | | | |
| 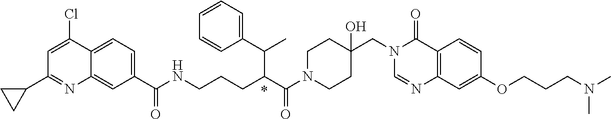 [SMILES: CC(c1ccccc1)C(CCCNC(=O)c1ccc2c(Cl)cc(C3CC3)nc2c1)C(=O)N1CCC(O)(Cn2cnc3cc(OCCCN(C)C)ccc3c2=O)CC1] | +++ | 7.27 | ND |
| (absolute stereochemistry not verified; opposite stereochemistry to 133) | | | |
| 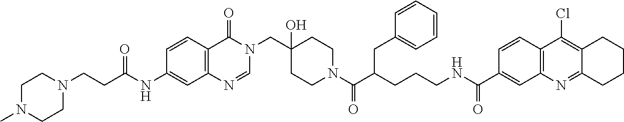 [SMILES: CN1CCN(CCC(=O)Nc2ccc3c(=O)n(CC4(O)CCN(C(=O)C(CCCNC(=O)c5ccc6c(Cl)c7c(nc6c5)CCCC7)Cc5ccccc5)CC4)cnc3c2)CC1] | ++++ | NA | NA |
| 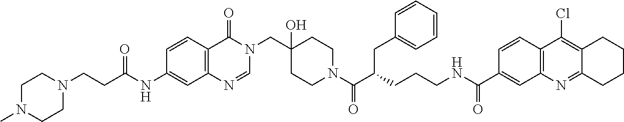 [SMILES: CN1CCN(CCC(=O)Nc2ccc3c(=O)n(CC4(O)CCN(C(=O)[C@@H](CCCNC(=O)c5ccc6c(Cl)c7c(nc6c5)CCCC7)Cc5ccccc5)CC4)cnc3c2)CC1] | ++++ | 2 | >500 |
| (stereochemistry not verified; opposite stereochemistry as next compound) | | | |
| 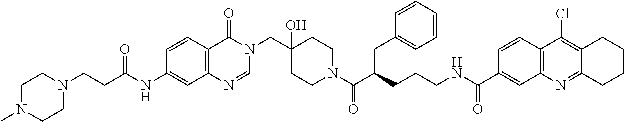 [SMILES: CN1CCN(CCC(=O)Nc2ccc3c(=O)n(CC4(O)CCN(C(=O)[C@H](CCCNC(=O)c5ccc6c(Cl)c7c(nc6c5)CCCC7)Cc5ccccc5)CC4)cnc3c2)CC1] | ++ | NA | NA |
| (stereochemistry not verified; opposite stereochemistry to previous compound) | | | |
| 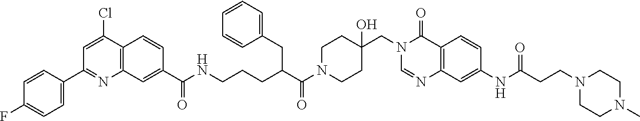 [SMILES: CN1CCN(CCC(=O)Nc2ccc3c(=O)n(CC4(O)CCN(C(=O)C(CCCNC(=O)c5ccc6c(Cl)cc(-c7ccc(F)cc7)nc6c5)Cc5ccccc5)CC4)cnc3c2)CC1] | ++ | 22.7 | NA |
| 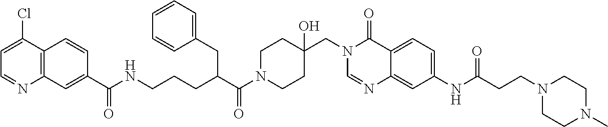 [SMILES: CN1CCN(CCC(=O)Nc2ccc3c(=O)n(CC4(O)CCN(C(=O)C(CCCNC(=O)c5ccc6c(Cl)ccnc6c5)Cc5ccccc5)CC4)cnc3c2)CC1] | +++ | 11.0 | 327.6 |
| 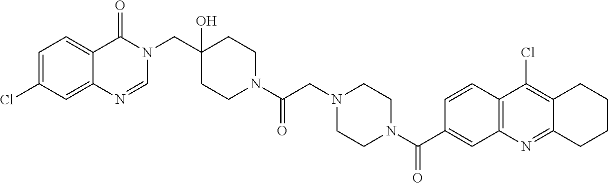 [SMILES: O=C(CN1CCN(C(=O)c2ccc3c(Cl)c4c(nc3c2)CCCC4)CC1)N1CCC(O)(Cn2cnc3cc(Cl)ccc3c2=O)CC1] | + | 2.31 | NA |
| 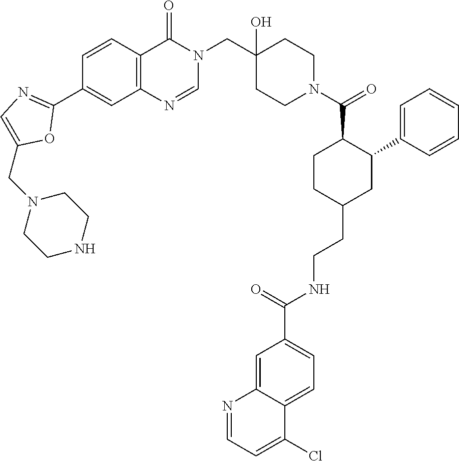 [SMILES: O=C(NCCC1CC[C@@H](C(=O)N2CCC(O)(Cn3cnc4cc(-c5ncc(CN6CCNCC6)o5)ccc4c3=O)CC2)[C@H](c2ccccc2)C1)c1ccc2c(Cl)ccnc2c1] | +++ | ND | ND |
| 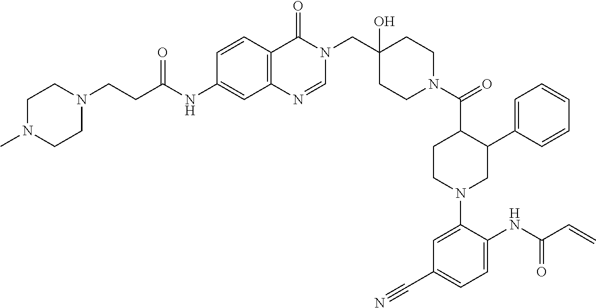 [SMILES: C=CC(=O)Nc1ccc(C#N)cc1N1CCC(C(=O)N2CCC(O)(Cn3cnc4cc(NC(=O)CCN5CCN(C)CC5)ccc4c3=O)CC2)C(c2ccccc2)C1] | + | ND | ND |
| 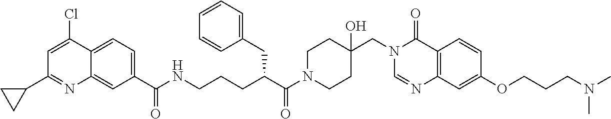 [SMILES: CN(C)CCCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1] | ++++ | 27.08 | ∞* |
| 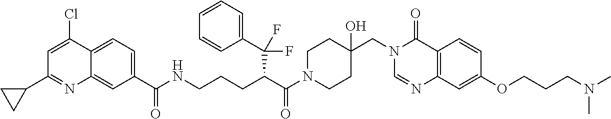 [SMILES: CN(C)CCCOc1ccc2c(=O)n(CC3(O)CCN(C(=O)[C@H](CCCNC(=O)c4ccc5c(Cl)cc(C6CC6)nc5c4)C(F)(F)c4ccccc4)CC3)cnc2c1] | ND | ND | ND |
| 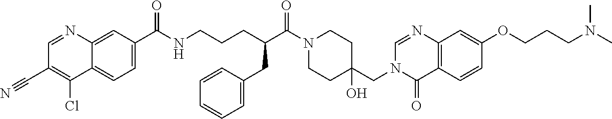 [SMILES: CN(C)CCCOc1ccc2c(=O)n(CC3(O)CCN(C(=O)[C@H](CCCNC(=O)c4ccc5c(Cl)c(C#N)cnc5c4)Cc4ccccc4)CC3)cnc2c1] | ++++ | 1.67 | ND |
| 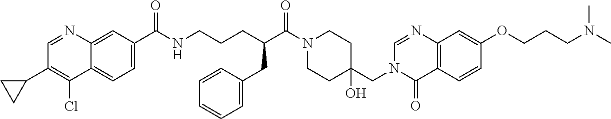 [SMILES: CN(C)CCCOc1ccc2c(=O)n(CC3(O)CCN(C(=O)[C@H](CCCNC(=O)c4ccc5c(Cl)c(C6CC6)cnc5c4)Cc4ccccc4)CC3)cnc2c1] | ++++ | 31.86 | ND |
| 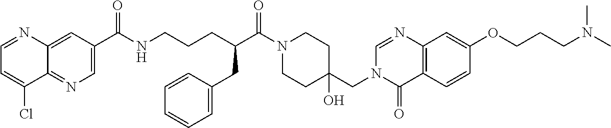 [SMILES: CN(C)CCCOc1ccc2c(=O)n(CC3(O)CCN(C(=O)[C@H](CCCNC(=O)c4cnc5c(Cl)ccnc5c4)Cc4ccccc4)CC3)cnc2c1] | ++ | 43.96 | ND |
| 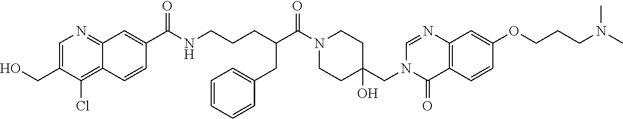 [SMILES: CN(C)CCCOc1ccc2c(=O)n(CC3(O)CCN(C(=O)C(CCCNC(=O)c4ccc5c(Cl)c(CO)cnc5c4)Cc4ccccc4)CC3)cnc2c1] | ++++ | 57.64 | ND |
| 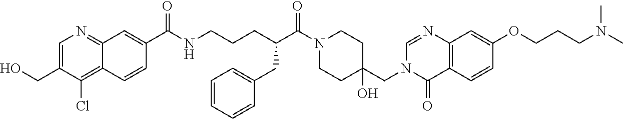 [SMILES: CN(C)CCCOc1ccc2c(=O)n(CC3(O)CCN(C(=O)[C@@H](CCCNC(=O)c4ccc5c(Cl)c(CO)cnc5c4)Cc4ccccc4)CC3)cnc2c1] | ++++ | ND | ND |
| 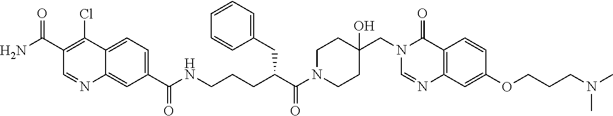 [SMILES: CN(C)CCCOc1ccc2c(=O)n(CC3(O)CCN(C(=O)[C@H](CCCNC(=O)c4ccc5c(Cl)c(C(N)=O)cnc5c4)Cc4ccccc4)CC3)cnc2c1] | ++++ | 46.65 | 111.74 |
| 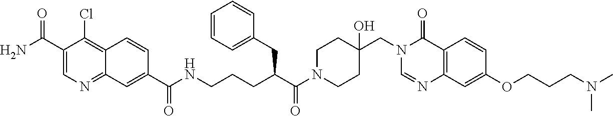 [SMILES: CN(C)CCCOc1ccc2c(=O)n(CC3(O)CCN(C(=O)[C@@H](CCCNC(=O)c4ccc5c(Cl)c(C(N)=O)cnc5c4)Cc4ccccc4)CC3)cnc2c1] | ++++ | 30.25 | |
| (stereochemistry not verified; opposite stereochemistry of next compound) | | | |
| 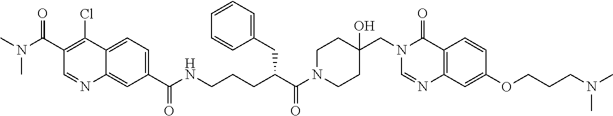 | | | |
| (stereochemistry not verified; opposite stereochemistry of previous compound) | ++ | 33.75 | |
| 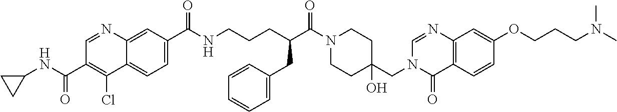 [SMILES: CN(C)CCCOc1ccc2c(=O)n(CC3(O)CCN(C(=O)[C@H](CCCNC(=O)c4ccc5c(Cl)c(C(=O)NC6CC6)cnc5c4)Cc4ccccc4)CC3)cnc2c1] | ++++ | 44.14 | 28.89 |
| (stereochemistry not verified; opposite stereochemistry of next compound) | | | |
| 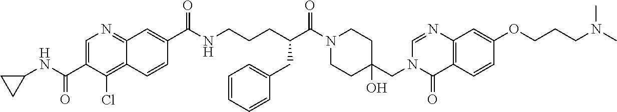 [SMILES: CN(C)CCCOc1ccc2c(=O)n(CC3(O)CCN(C(=O)[C@@H](CCCNC(=O)c4ccc5c(Cl)c(C(=O)NC6CC6)cnc5c4)Cc4ccccc4)CC3)cnc2c1] | ++++ | 62.44 | |
| (stereochemistry not verified; opposite stereochemistry of previous compound) | | | |
| 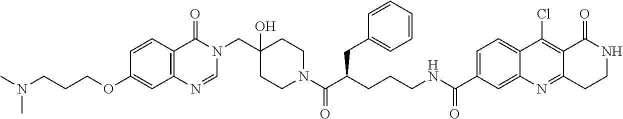 [SMILES: CN(C)CCCOc1ccc2c(=O)n(CC3(O)CCN(C(=O)[C@H](CCCNC(=O)c4ccc5c(Cl)c6c(nc5c4)CCNC6=O)Cc4ccccc4)CC3)cnc2c1] | ++ | 10.17 | NA |
| 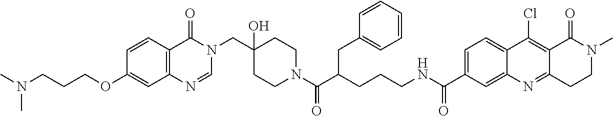 | +++ | 14.43 | NA |
| 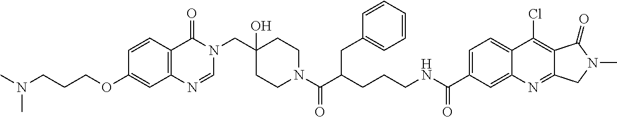 | ++++ | 11.27 | NA |
| 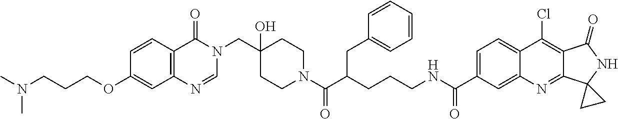 [SMILES: CN(C)CCCOc1ccc2c(=O)n(CC3(O)CCN(C(=O)C(CCCNC(=O)c4ccc5c(Cl)c6c(nc5c4)C4(CC4)NC6=O)Cc4ccccc4)CC3)cnc2c1] | ND | ND | ND |
| 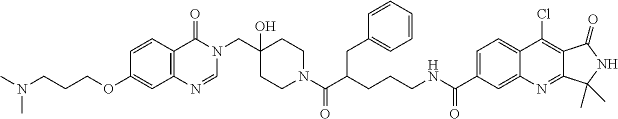 | + | 16.3 | NA |
| 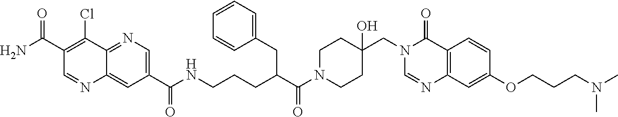 [SMILES: CN(C)CCCOc1ccc2c(=O)n(CC3(O)CCN(C(=O)C(CCCNC(=O)c4cnc5c(Cl)c(C(N)=O)cnc5c4)Cc4ccccc4)CC3)cnc2c1] | ND | ND | ND |
| 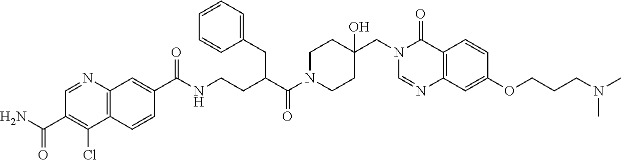 [SMILES: CN(C)CCCOc1ccc2c(=O)n(CC3(O)CCN(C(=O)C(CCNC(=O)c4ccc5c(Cl)c(C(N)=O)cnc5c4)Cc4ccccc4)CC3)cnc2c1] | ++++ | 28.21 | NA |
| 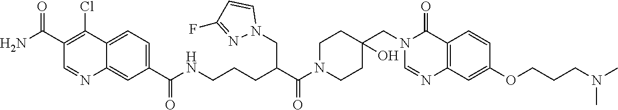 [SMILES: CN(C)CCCOc1ccc2c(=O)n(CC3(O)CCN(C(=O)C(CCCNC(=O)c4ccc5c(Cl)c(C(N)=O)cnc5c4)Cn4ccc(F)n4)CC3)cnc2c1] | ++++ | 130.56 | 85.92 |
| 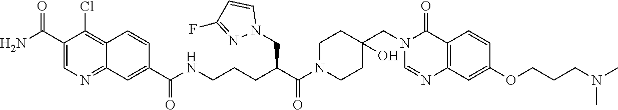 [SMILES: CN(C)CCCOc1ccc2c(=O)n(CC3(O)CCN(C(=O)[C@@H](CCCNC(=O)c4ccc5c(Cl)c(C(N)=O)cnc5c4)Cn4ccc(F)n4)CC3)cnc2c1] | ++++ | 223.19 | 85.92 |
| 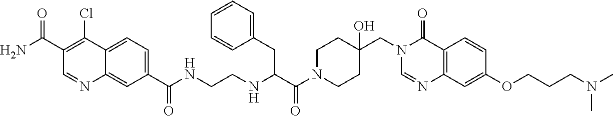 [SMILES: CN(C)CCCOc1ccc2c(=O)n(CC3(O)CCN(C(=O)C(Cc4ccccc4)NCCNC(=O)c4ccc5c(Cl)c(C(N)=O)cnc5c4)CC3)cnc2c1] | ++++ | 65.45 | |
| 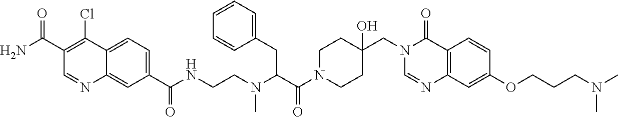 [SMILES: CN(C)CCCOc1ccc2c(=O)n(CC3(O)CCN(C(=O)C(Cc4ccccc4)N(C)CCNC(=O)c4ccc5c(Cl)c(C(N)=O)cnc5c4)CC3)cnc2c1] | ++++ | 36.41 | |
| 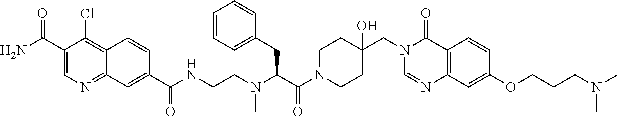 [SMILES: CN(C)CCCOc1ccc2c(=O)n(CC3(O)CCN(C(=O)[C@H](Cc4ccccc4)N(C)CCNC(=O)c4ccc5c(Cl)c(C(N)=O)cnc5c4)CC3)cnc2c1] | + | 100.97 | 148.99 |
| 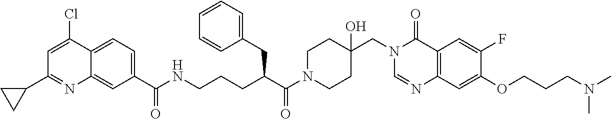 [SMILES: CN(C)CCCOc1cc2ncn(CC3(O)CCN(C(=O)[C@@H](CCCNC(=O)c4ccc5c(Cl)cc(C6CC6)nc5c4)Cc4ccccc4)CC3)c(=O)c2cc1F] | ++++ | 22.09 | |
| 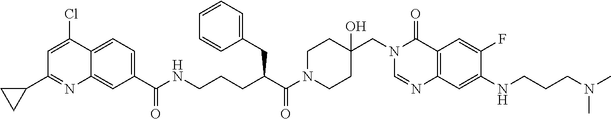 [SMILES: CN(C)CCCNc1cc2ncn(CC3(O)CCN(C(=O)[C@@H](CCCNC(=O)c4ccc5c(Cl)cc(C6CC6)nc5c4)Cc4ccccc4)CC3)c(=O)c2cc1F] | ++++ | 18.56 | |
| 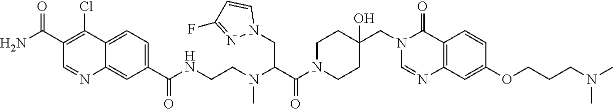 [SMILES: CN(C)CCCOc1ccc2c(=O)n(CC3(O)CCN(C(=O)C(Cn4ccc(F)n4)N(C)CCNC(=O)c4ccc5c(Cl)c(C(N)=O)cnc5c4)CC3)cnc2c1] | +++ | 106.24 | |
| 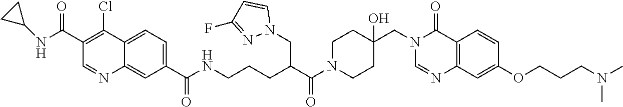 [SMILES: CN(C)CCCOc1ccc2c(=O)n(CC3(O)CCN(C(=O)C(CCCNC(=O)c4ccc5c(Cl)c(C(=O)NC6CC6)cnc5c4)Cn4ccc(F)n4)CC3)cnc2c1] | ++++ | 151.51 | |
| 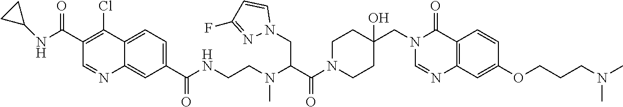 [SMILES: CN(C)CCCOc1ccc2c(=O)n(CC3(O)CCN(C(=O)C(Cn4ccc(F)n4)N(C)CCNC(=O)c4ccc5c(Cl)c(C(=O)NC6CC6)cnc5c4)CC3)cnc2c1] | ND | ND | ND |
| 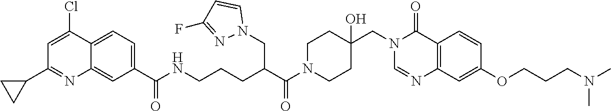 [SMILES: CN(C)CCCOc1ccc2c(=O)n(CC3(O)CCN(C(=O)C(CCCNC(=O)c4ccc5c(Cl)cc(C6CC6)nc5c4)Cn4ccc(F)n4)CC3)cnc2c1] | ++++ | 16.99 | 188.25 |
| 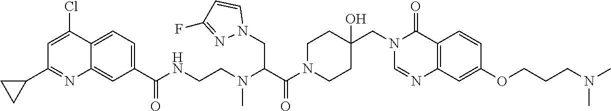 [SMILES: CN(C)CCCOc1ccc2c(=O)n(CC3(O)CCN(C(=O)C(Cn4ccc(F)n4)N(C)CCNC(=O)c4ccc5c(Cl)cc(C6CC6)nc5c4)CC3)cnc2c1] | ++ | NA | NA |
| 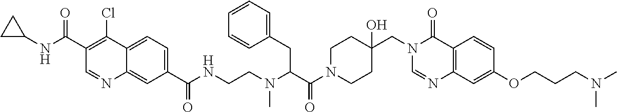 [SMILES: CN(C)CCCOc1ccc2c(=O)n(CC3(O)CCN(C(=O)C(Cc4ccccc4)N(C)CCNC(=O)c4ccc5c(Cl)c(C(=O)NC6CC6)cnc5c4)CC3)cnc2c1] | +++ | 46.55 | NA |
| 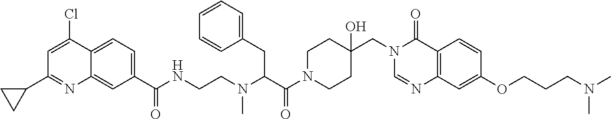 [SMILES: CN(C)CCCOc1ccc2c(=O)n(CC3(O)CCN(C(=O)C(Cc4ccccc4)N(C)CCNC(=O)c4ccc5c(Cl)cc(C6CC6)nc5c4)CC3)cnc2c1] | ++ | NA | NA |
| 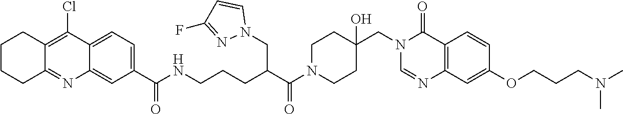 [SMILES: CN(C)CCCOc1ccc2c(=O)n(CC3(O)CCN(C(=O)C(CCCNC(=O)c4ccc5c(Cl)c6c(nc5c4)CCCC6)Cn4ccc(F)n4)CC3)cnc2c1] | ++++ | 2.46 | 396.23 |
| 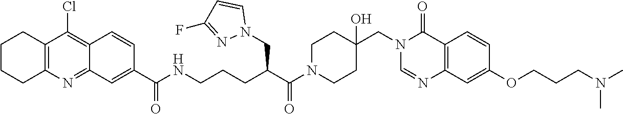 [SMILES: CN(C)CCCOc1ccc2c(=O)n(CC3(O)CCN(C(=O)[C@@H](CCCNC(=O)c4ccc5c(Cl)c6c(nc5c4)CCCC6)Cn4ccc(F)n4)CC3)cnc2c1] | ND | 6.96 | 127.44 |
| 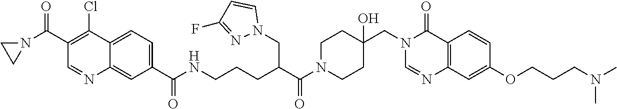 [SMILES: CN(C)CCCOc1ccc2c(=O)n(CC3(O)CCN(C(=O)C(CCCNC(=O)c4ccc5c(Cl)c(C(=O)N6CC6)cnc5c4)Cn4ccc(F)n4)CC3)cnc2c1] | ND | ND | ND |
| 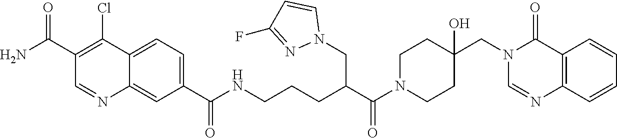 [SMILES: NC(=O)c1cnc2cc(C(=O)NCCCC(Cn3ccc(F)n3)C(=O)N3CCC(O)(Cn4cnc5ccccc5c4=O)CC3)ccc2c1Cl] | +++ | 6.51 | ND |
| 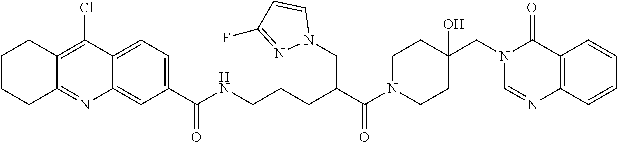 [SMILES: O=C(NCCCC(Cn1ccc(F)n1)C(=O)N1CCC(O)(Cn2cnc3ccccc3c2=O)CC1)c1ccc2c(Cl)c3c(nc2c1)CCCC3] | ++++ | 0.56 | 393.74 |
| 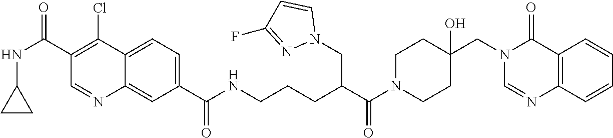 [SMILES: O=C(NCCCC(Cn1ccc(F)n1)C(=O)N1CCC(O)(Cn2cnc3ccccc3c2=O)CC1)c1ccc2c(Cl)c(C(=O)NC3CC3)cnc2c1] | +++ | 4.61 | ND |
| 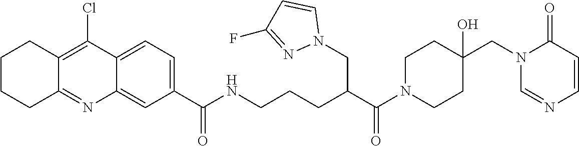 [SMILES: O=C(NCCCC(Cn1ccc(F)n1)C(=O)N1CCC(O)(Cn2cnccc2=O)CC1)c1ccc2c(Cl)c3c(nc2c1)CCCC3] | ND | ND | ND |

### USP7 Assay Protocol

### 1. Materials:

USP7, Active human recombinant protein (CP, Lot. 20200225001), Ubiquitin-Rhodamine 110 (Rh110) (BostonBiochem, Cat. No. U-555), DMSO (Sigma, Cat. N34869), DTT (Sangon Biotech, Cat. No. A620058-0005), XM Tris pH 8.0 (Sigma, Cat. No. T2694-1L), Tween-20 (Sigma, Cat. No. P2287-100ML), EDTA (Invitrogen, Cat. No. 15575020, BSA (Sigma, Cat. No. B2064-100G), 384-well assay plate (Corning, Cat. No. 3573), Echo Qualified 384-Well Polypropylene Microplate 2.0, Clear, Flat Bottom (LABCYTE, Cat. No. 001-14555).

### 2. Instrument Information:

| Name | Model | Manufacturer | SN |
|---|---|---|---|
| Precision | PRC384U | BioTek | 214145 |
| Echo 550 Liquid Handler | Echo 550 | Labcyte | E5XX-1045 |
| Centrifuge | Avanti J-15R | BECKMAN | JBR20A055 |
| | | COULTER | |
| Multi-Mode | SpectraMax | Molecular | 33270-2051 |
| Detection Plaform | Paradigm | | |

### 3. Experimental Methods:

Dilute the compounds to 400× of the final desired highest inhibitor concentration in reaction by 100% DMSO. For all compounds, transfer the compounds to one well in a 384-well Echo plate and serially dilute the compound by 3-fold dilution of 100% DMSO in the next well and so forth for a total of 10 concentrations by Precision. Add 30 μL of 100% DMSO to two empty wells for no compound control and no enzyme control in the same 384-well Echo plate. Make the plate as a source plate.

For USP7 assay, transfer 50 nL of each well from the source plate to a 384-well assay plate (OptiPlate TM-384 F black assay plates) by Echo 550 Liquid Handler.

Prepare 1× assay buffer:

| 50 mM | Tris-HCl pH 8.0 |
|---|---|
| 0.5 mM | EDTA |
| 0.01% | BSA |
| 0.01% | Tween-20 |

Added appropriate amount of USP7 in 1× assay Buffer to prepare 2× enzyme solution (USP7 final concentration: 0.05 nM). Added appropriate amount of Ubiquitin-Rhodamine 110 in 1× assay Buffer to prepare 2× substrate solution (Ubiquitin-Rhodamine 110 final concentration: 190 nM). Added 10 μL of 2× enzyme solution to each reaction well of the 384-well assay plate, except for control wells without enzyme (add 10 μL of 1× Assay Buffer instead as for low control). Centrifuged at 1000 rpm for 1 min, and incubate at RT for 15 min. Transferred 2× substrate solution to the assay plate. Added 10 μL of 2× substrate solution to each well of the 384-well assay plate to start reaction. Centrifuged at 1000 rpm for 1 min. Read the plate kinetically on SpectraMax Paradigm for 20 min with excitation at 485 nm and emission at 535 nm. Copied raw data (slope) from Reader to obtain inhibition values in Excel using equation (1):

$$Inh \% = ( Max - Signal ) / ( Max - Min ) ⋆ 100 \qquad Equation ( 1 )$$

Max signal was obtained from the action of Enzyme and Substrate. Min signal was obtained from the Substrate only. To fit the data in XLFit excel add-in version 5.4.0.8 to obtain IC₅₀ values used equation (2):

$$Y = Bottom + ( Top - Bottom ) / ( 1 + ( ( I {C}_{50} / X ) ⋆ HillSlope ) ) \qquad Equation ( 2 )$$

Y is % inhibition and X is compound concentration.

### Plasma Assay Protocol

1. Preheated 0.05 M sodium phosphate and 0.07 M NaCl buffer, pH 7.4. Dissolved 14.505 g/L Na₂HPO₄·12H₂O, 1.483 g/L NaH₂PO₄·2H₂O and 4.095 g/L NaCl in deionized water. The basic solution was then titrated with the phosphoric acid to pH 7.40. Stored in fridge up to 7 days. Checked pH on the day of experiment and adjust if outside specification of 7.4+/−0.1.
2. Plasma preparation:
Thawed frozen plasma by placing at 37° C. quickly. Centrifuged plasma at 3,000 rpm for 8 minutes to remove clots, pipetted and pooled the supernatant as the plasma to be used in the experiment. Checked and recorded the pH of the plasma. Only used plasma within the range of pH 7.4 to pH 8. If higher than pH 8, discarded the plasma. By using a 5% CO₂ incubator and PBS buffer, a pH of 7.4 was reached after the 4 hour equilibrium dialysis time. Put the plasma on ice until used.
3. Tested compounds and reference compounds spiking solution:
0.5 mM test compounds spiking solution A: Added 10 μL of 10 mM test compounds stock solution to 190 μL DMSO.
0.02 mM spiking solution B: Added 40 μL of spiking solution A to 960 μL of 0.05 mM Sodium phosphate buffer with 0.5% BSA.

Pre-warmed the plasma and spiking solution B at 37° C. for 5 min. Added 10 μL of pre-warmed spiking solution B into the wells designated for all the time points (5, 15, 30, 45, 60 min).

For 0-min, added 400 μL of acetonitrile (ACN) containing the internal standard (IS) to the wells of 0-min plate and then added 90 μL of plasma. Added 90 μL of pre-warmed plasma into the wells designated for the time points (0, 5, 15, 30, 45, 60 min), and started timing. At 5, 15, 30, 45, 60 min, added 400 μL of ACN containing IS to the wells of corresponding plates, respectively, to stop the reaction. After quenching, shook the plates at the vibrator (IKA, MTS 2/4) for 10 min (600 rpm/min) and then centrifuged at 5594 g for 15 min (Thermo Multifuge×3R). Transferred 50 μL of the supernatant from each well into a 96-well sample plate containing 50 μL of ultra pure water (Millipore ZMQS50F01) for LC/MS analysis.

### Microsome Assay Protocol

1. Buffer A: 1.0 L of 0.1 M monobasic Potassium Phosphate buffer containing 1.0 mM EDTA Buffer B: 1.0 L of 0.1 M Dibasic Potassium Phosphate buffer containing 1.0 mM EDTA
Buffer C: 0.1 M Potassium Phosphate buffer, 1.0 mM EDTA, pH 7.4 by titrating 700 mL of buffer B with buffer A while monitoring with a pH meter.
2. Reference compound (Ketanserin) and test compounds spiking solution:
500 μM spiking solution: added 10 μL of 10 mM DMSO stock solution into 190 μL ACN.
1.5 μM spiking solution in microsomes (0.75 mg/mL): added 1.5 μL of 500 μM spiking solution and 18.75 μL of 20 mg/mL liver microsomes into 479.75 μL of Buffer C on ice.
3. Prepared NADPH stock solution (6 mM) by dissolving NADPH into buffer C. Dispensed 30 μL of 1.5 μM spiking solution containing 0.75 mg/mL microsomes solution to the assay plates designated for different time points (0-, 5-, 15-, 30-, 45-min) on ice. For 0-min, added 135 μL of ACN containing IS to the wells of 0-min plate and then added 15 μL of NADPH stock solution (6 mM). Pre-incubated all other plates at 37° C. for 5 minutes. Added 15 μL of NADPH stock solution (6 mM) to the plates to start the reaction and timing. At 5-min, 15-min, 30-min, and 45-min, added 135 μL of ACN containing IS to the wells of corresponding plates, respectively, to stop the reaction. After quenching, shook the plates at the vibrator (IKA, MTS 2/4) for 10 min (600 rpm/min) and then centrifuged at 5594 g for 15 min (Thermo Multifuge×3R). Transferred 50 μL of the supernatant from each well into a 96-well sample plate containing 50 μL of ultra pure water (Millipore, ZMQS50F01) for LC/MS analysis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of Formula (I): 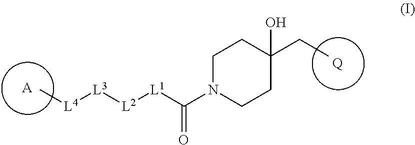
or a pharmaceutically acceptable salt thereof, wherein:
Q is 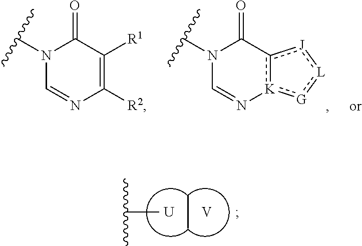
G is CR¹⁰, NR¹⁰, or N;
J is CR¹⁰, NR¹⁰, N, or S;
L is NR¹⁰, CR¹⁰, CR¹¹ or N;
K is C or N; [symbol]
is a single bond or a double bond, when valence permits, provided the ring containing J, L, and G is aromatic;
U is a 5-6 membered heterocyclyl optionally substituted with one or more Hal, —NH₂, —CN, —CF₃, —OH, oxo, or C₁₋₃ alkyl;
V is a 5-6 membered heteroaryl or a C₃₋₆ cycloalkyl, wherein the 5-6 membered heteroaryl or the C₅₋₆ cycloalkyl is optionally substituted with one or more Hal, —NH₂, —CN, —CF₃, —OH, oxo, or C₁₋₃ alkyl;
R¹ is H;
R² is H or a —NHC₁₋₃ alkylene(C₅₋₇ heterocyclyl),
or R¹ and R² taken together with the carbon atoms to which they are attached form a C₆₋₁₀ aryl or a 5-6 membered heteroaryl, wherein the C₆₋₁₀ aryl or the 5-6 membered heteroaryl is optionally substituted with one or more substituents independently selected from Hal, C₁₋₃ alkyl, C₁₋₃ alkoxy, C₆₋₁₀ aryl, 5-6 membered heteroaryl, 4-7 membered heterocyclyl, —NHC(═O)C₁₋₃ alkylene(NR³R⁴), —C(═O)NHC₁₋₃ alkylene(NR³R⁴), —NHC₁₋₃ alkylene(4-7 membered heterocyclyl), —OC₁₋₃ alkylene(4-7 membered heterocyclyl), —OC₁₋₃ alkylene-O—(NR³R⁴), —NHC₁₋₃alkylene(NR³R⁴), or —OC₁₋₃ alkylene(NR³R⁴), and wherein the C₁₋₃ alkyl, the C₆₋₁₀ aryl, the 5-6 membered heteroaryl, or the 4-7 membered heterocyclyl is optionally independently substituted with one or more Hal, —NH₂, —CN, —CF₃, C₁₋₃ alkyl, or —C₀₋₂ alkyl(5-6 membered heterocyclyl);
R³ is a C₁₋₃ alkyl;
R⁴ is a C₁₋₃ alkyl,
or R³ and R⁴ is together with the nitrogen atom to which they are attached form a 5-7 membered heterocyclyl or a 5-7 membered heteroaryl, wherein the 5-7 membered heterocyclyl is optionally substituted with one or more C₁₋₃ alkyl;
L¹ is a C₂₋₄ alkylene substituted with one or more —C₀₋₂ alkyl(C₆ aryl), —NHC₀₋₂ alkyl(C₆ aryl), or —C₀₋₂ alkyl(5-6 membered heteroaryl), wherein one or more carbons in the C₂₋₄ alkylene is optionally replaced with N, which is optionally substituted with C₁₋₃ alkyl; or L¹ is 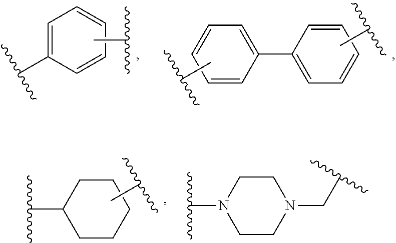
in either direction, 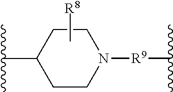
in either direction, or 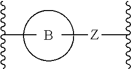
in either direction, wherein the —C₀₋₂ alkyl(C₆ aryl) or the —C₀₋₂ alkyl(5-6 membered heteroaryl) is optionally independently substituted with one or more Hal, —NH₂, —CN, —CF₃, or C₁₋₃ alkyl; Z is a C₁₋₄ alkylene, and [symbol] is a C₆₋₁₀ arylene, 5-7 membered heterocyclylene or a 5-7 membered heteroarylene, and wherein the C₁₋₄ alkylene, the 5-7 membered heterocyclylene, and the 5-7 membered heteroarylene is each optionally independently substituted with a C₀₋₂ alkyl(C₆ aryl) or a C₃₋₆ cycloalkyl;
L² is —NH—, a bond, or 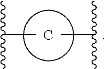
wherein [symbol] is a 5 membered heteroarylene;
L³ is —C(═O)—, —S(═O)₂—, a C₆₋₁₀ arylene, or a bond;
L⁴ is a C₁₋₃ alkylene, a C₂₋₄ alkenylene, or a bond; [symbol]
is H, 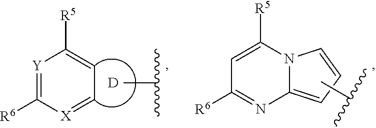
a C₆₋₁₀ aryl, or a 5-7 membered heteroaryl, wherein the C₆₋₁₀ aryl or the 5-7 membered heteroaryl is optionally substituted with one or more Hal, —CN, a C₁₋₃ haloalkyl, —NHC(═O)C₁₋₃ alkenyl;
X is N or CH;
Y is N or CR⁷;
R⁵ is Hal, —NH₂, —NH₂(C₁₋₃ alkyl), —OH, a C₁₋₃ alkoxy, or C₁₋₃ alkyl;
R⁶ is H, Hal, —NH₂, —OH, —C(O)NH₂, a C₁₋₃ alkyl, a C₁₋₃ alkoxy, a C₆₋₁₀ aryl, a 5-6 membered heteroaryl, or a C₃₋₅ cycloalkyl, wherein —NH₂, —OH, the C₁₋₃ alkyl, the C₆₋₁₀ aryl, the 5-6 membered heteroaryl, or the C₃₋₅ cycloalkyl is optionally substituted with one or more Hal, —NH₂, —CN, —CF₃, a C₁₋₃ alkyl, C₁₋₃ alkyl(NH₂), or C₁₋₃ alkyl(CF₃);
R⁷ is H, Hal, a C₁₋₃ alkyl, C₁₋₃ haloalkyl, cyano, C₃₋₅ cycloalkyl, —C(O)NH₂, —C(O)N(C₁₋₃ alkyl)₂, —C(O)NHC₃₋₅ cycloalkyl, or —C(═O)NHC₁₋₃ alkyl, wherein when R⁷ is —C(O)N(C₁₋₃ alkyl)₂ the two C₁₋₃ alkyl groups are optionally taken together with the N atom to which they are attached to form a 3-7 membered heterocyclyl;
or R⁷ and R⁶ taken together with the carbon atoms to which they are attached form a C₅₋₇ cycloalkyl, a C₆₋₈ bridged bicyclic cycloalkyl, or a 5-6 membered heterocyclyl, wherein the C₅₋₇ cycloalkyl or the 5-6 membered heterocyclyl is optionally substituted with one or more C₁₋₃ alkyl, oxo, or
—C(═O)C₁₋₃ alkyl, wherein two C₁₋₃ alkyl on the same carbon are optionally taken together with the carbon atom to which they are attached to form a spiro ring; [symbol]
is a C₆₋₁₀ aryl, a 5-6 membered heterocyclyl, or a 5-6 membered heteroaryl;
R⁸ is a C₆₋₁₀ aryl, optionally substituted with one or more Hal, —NH₂, —CN, —CF₃, a C₁₋₃ alkyl, C₁₋₃ alkyl(NH₂), or C₁₋₃ alkyl(CF₃);
R⁹ is a bond, a C₂₋₄ alkylene, a C₆₋₁₀ arylene, or 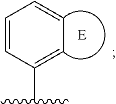 [symbol]
is a 5-6 membered heterocyclyl optionally substituted with one or more Hal, —NH₂, —CN,
—CF₃, a C₁₋₃ alkyl, C₁₋₃ alkyl(NH₂), or C₁₋₃ alkyl(CF₃);
R¹⁰ is H, C₁₋₃ alkylene(C₅₋₇ heterocyclyl), or a C₆₋₁₀ aryl, wherein C₁₋₃ alkylene(C₅₋₇ heterocyclyl) or C₆₋₁₀ aryl is optionally substituted with one or more Hal, —NH₂, —CN, —CF₃, a C₁₋₃ alkyl, C₁₋₃ alkyl(NH₂), C₁₋₃ alkyl(CF₃) or C₁₋₃ alkyl(NH₂)(CF₃); and
R¹¹ is Hal, —NH₂, —CN, —CF₃, or C₁₋₃ alkyl,
provided the compound is not

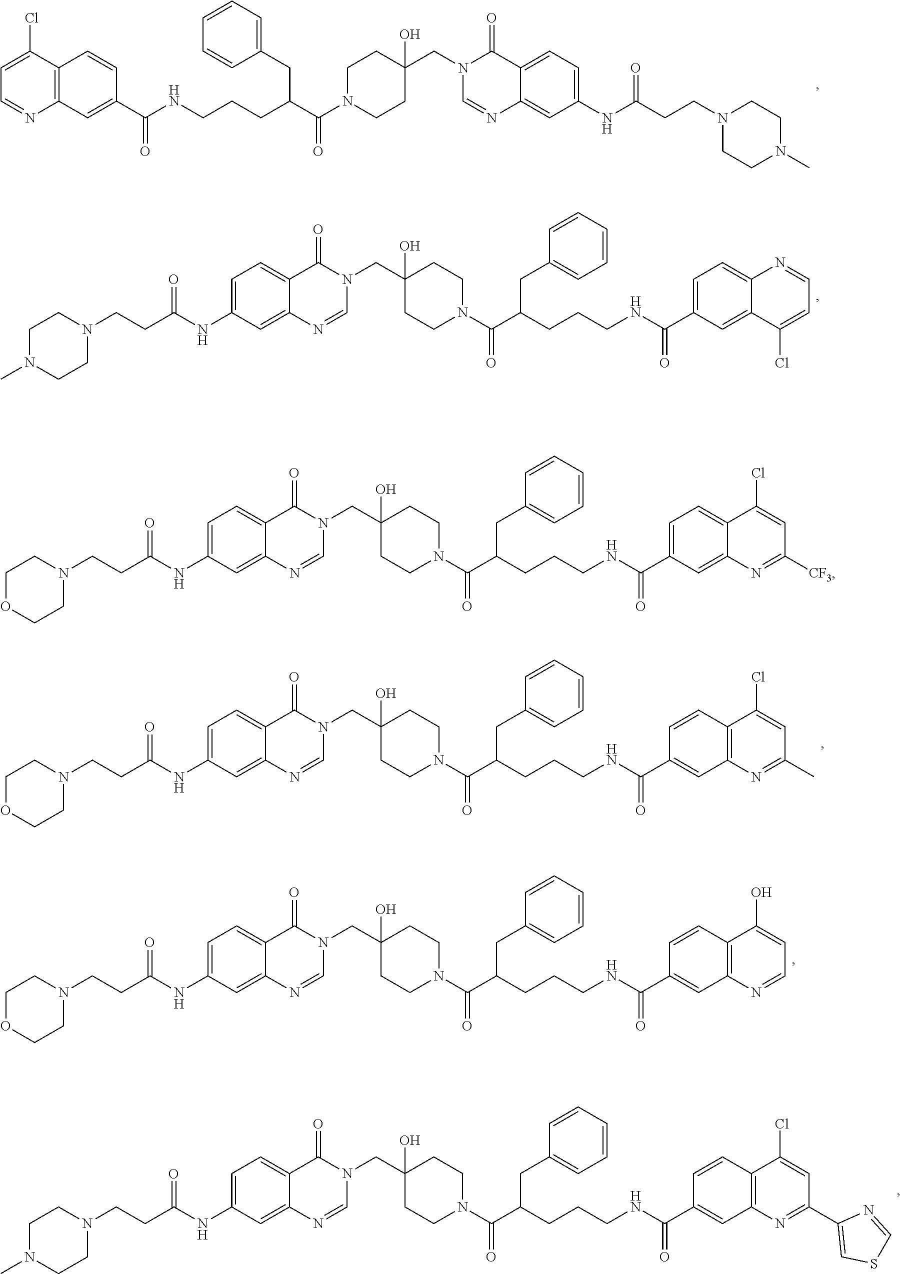

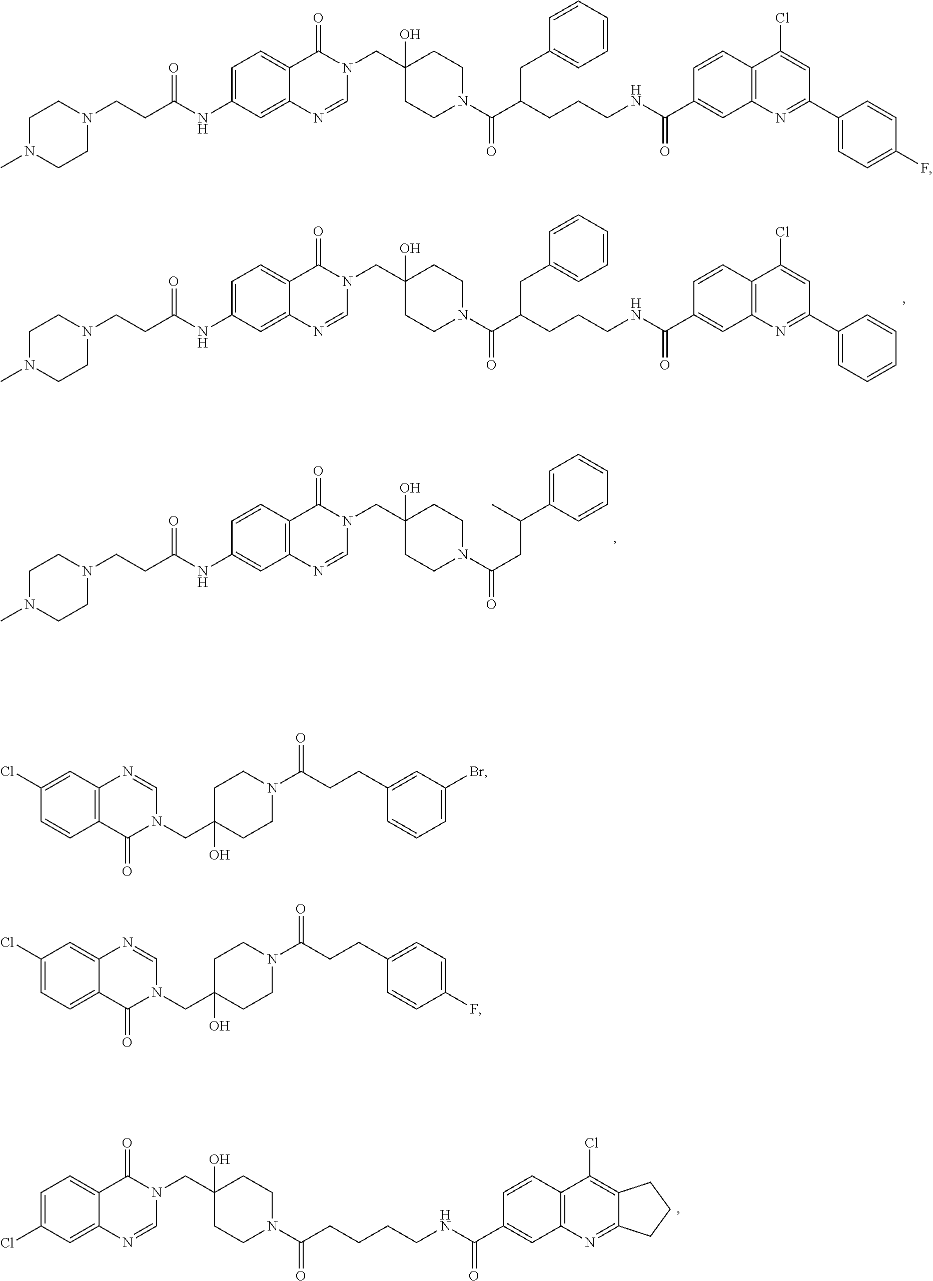 [SMILES: CN1CCN(CCC(=O)Nc2ccc3c(=O)n(CC4(O)CCN(C(=O)C(CCCNC(=O)c5ccc6c(Cl)cc(-c7ccc(F)cc7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7ccccc7)nc6c5)Cc5ccccc5)CC4)cnc3c2)CC1 ; CC(CC(=O)N1CCC(O)(Cn2cnc3cc(NC(=O)CCN4CCN(C)CC4)ccc3c2=O)CC1)c1ccccc1 ; O=C(CCc1cccc(Br)c1)N1CCC(O)(Cn2cnc3cc(Cl)ccc3c2=O)CC1 ; O=C(CCc1ccc(F)cc1)N1CCC(O)(Cn2cnc3cc(Cl)ccc3c2=O)CC1 ; O=C(NCCCCC(=O)N1CCC(O)(Cn2cnc3cc(Cl)ccc3c2=O)CC1)c1ccc2c(Cl)c3c(nc2c1)CCC3]

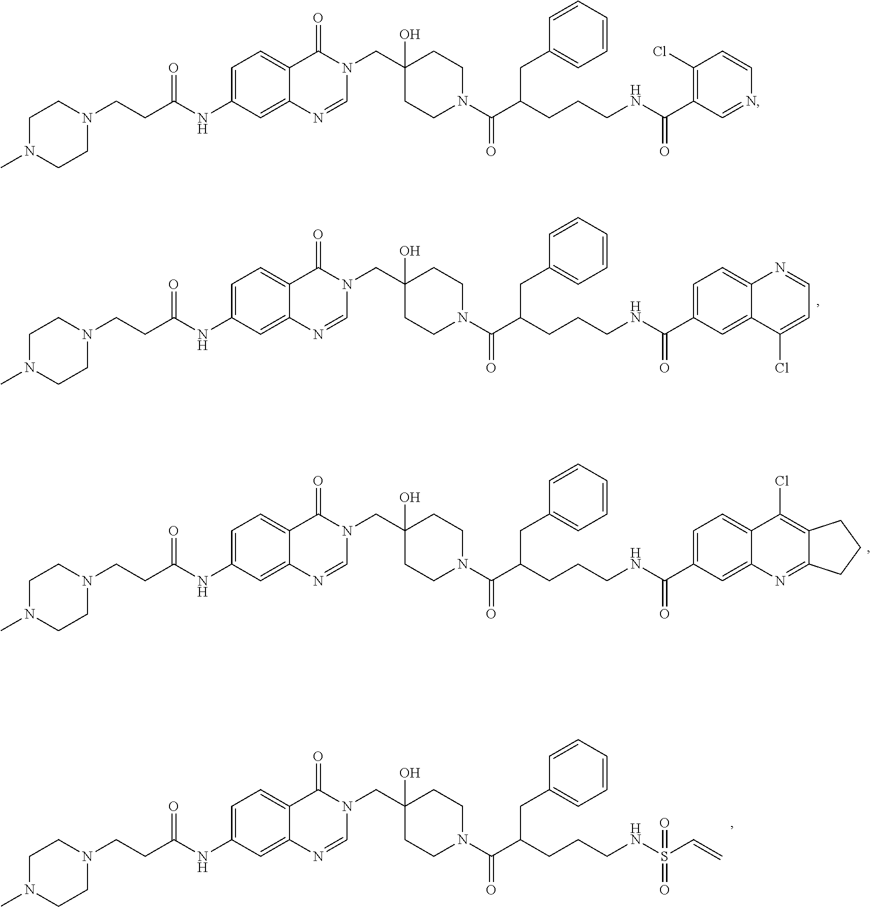 [SMILES: CN1CCN(CCC(=O)Nc2ccc3c(=O)n(CC4(O)CCN(C(=O)C(CCCNC(=O)c5cnccc5Cl)Cc5ccccc5)CC4)cnc3c2)CC1 ; CN1CCN(CCC(=O)Nc2ccc3c(=O)n(CC4(O)CCN(C(=O)C(CCCNC(=O)c5ccc6nccc(Cl)c6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)c7c(nc6c5)CCC7)Cc5ccccc5)CC4)cnc3c2)CC1 ; C=CS(=O)(=O)NCCCC(Cc1ccccc1)C(=O)N1CCC(O)(Cn2cnc3cc(NC(=O)CCN4CCN(C)CC4)ccc3c2=O)CC1]

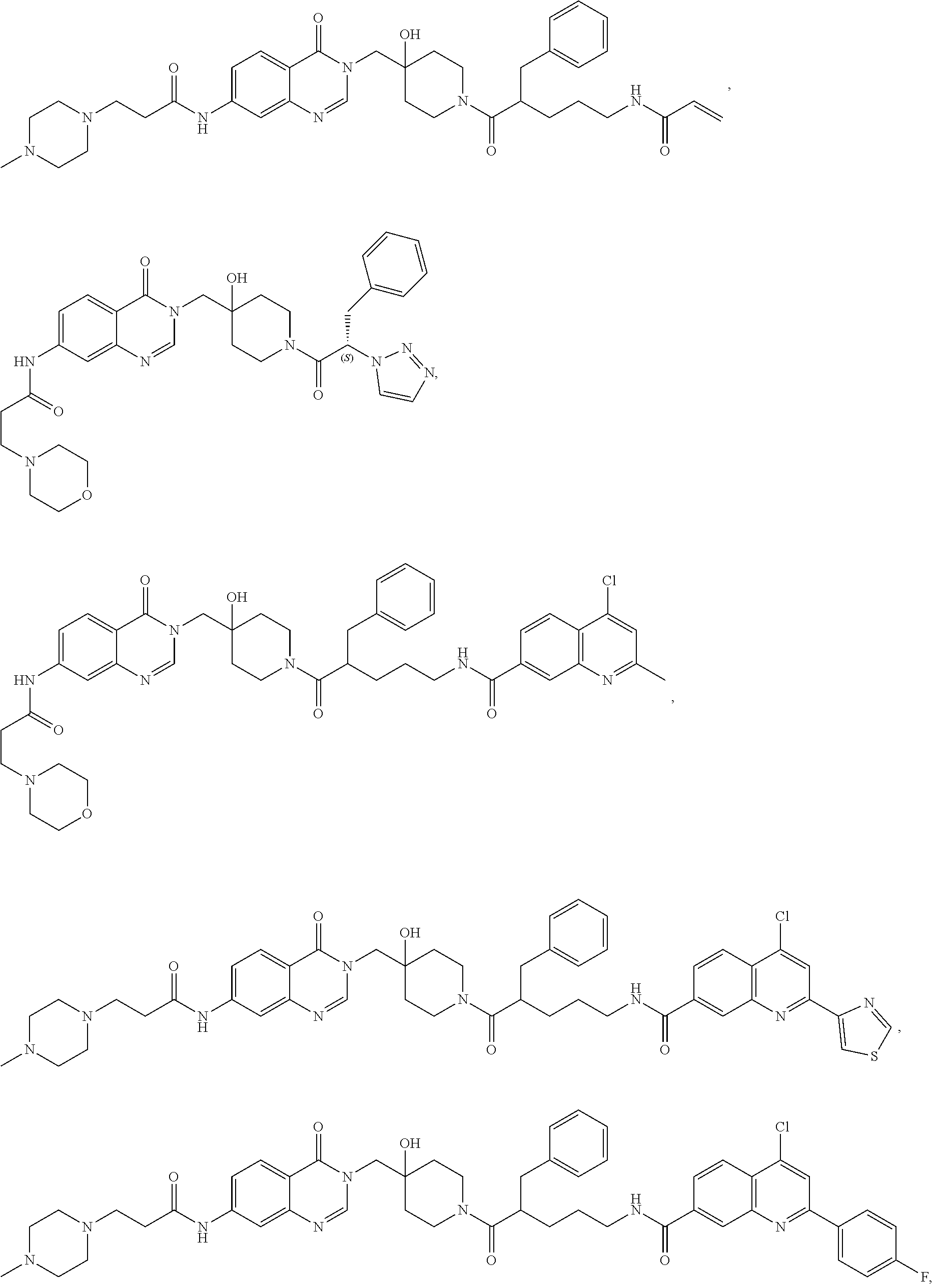

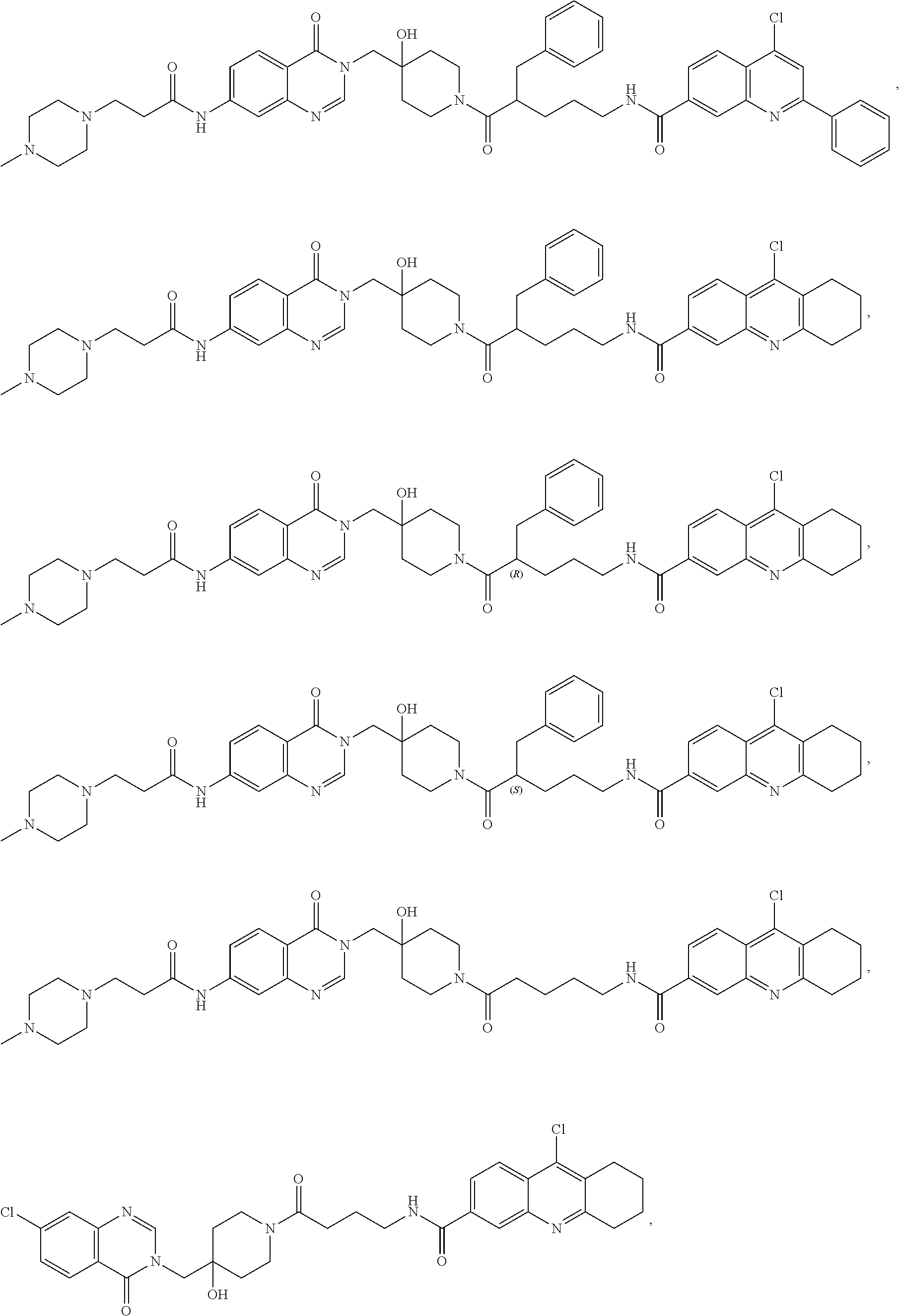

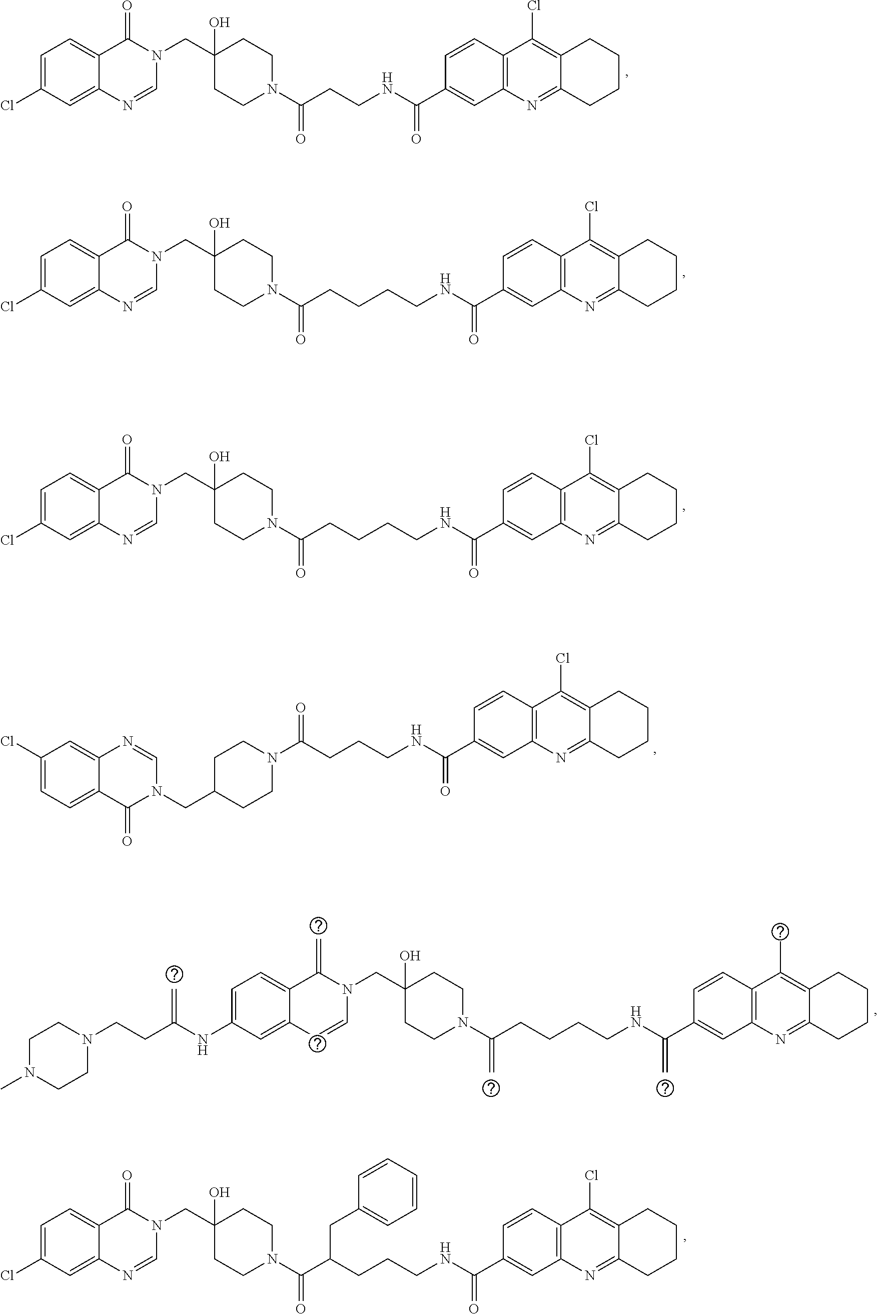 [SMILES: O=C(NCCC(=O)N1CCC(O)(Cn2cnc3cc(Cl)ccc3c2=O)CC1)c1ccc2c(Cl)c3c(nc2c1)CCCC3 ; O=C(NCCCCC(=O)N1CCC(O)(Cn2cnc3cc(Cl)ccc3c2=O)CC1)c1ccc2c(Cl)c3c(nc2c1)CCCC3 ; O=C(NCCCCC(=O)N1CCC(O)(Cn2cnc3cc(Cl)ccc3c2=O)CC1)c1ccc2c(Cl)c3c(nc2c1)CCCC3 ; O=C(NCCCC(=O)N1CCC(Cn2cnc3cc(Cl)ccc3c2=O)CC1)c1ccc2c(Cl)c3c(nc2c1)CCCC3 ; C=C(CCN1CCN(C)CC1)Nc1ccc2c(c1)C=CN(CC1(O)CCN(C(=C)CCCCNC(=C)c3ccc4c(C)c5c(nc4c3)CCCC5)CC1)C2=C ; O=C(NCCCC(Cc1ccccc1)C(=O)N1CCC(O)(Cn2cnc3cc(Cl)ccc3c2=O)CC1)c1ccc2c(Cl)c3c(nc2c1)CCCC3]

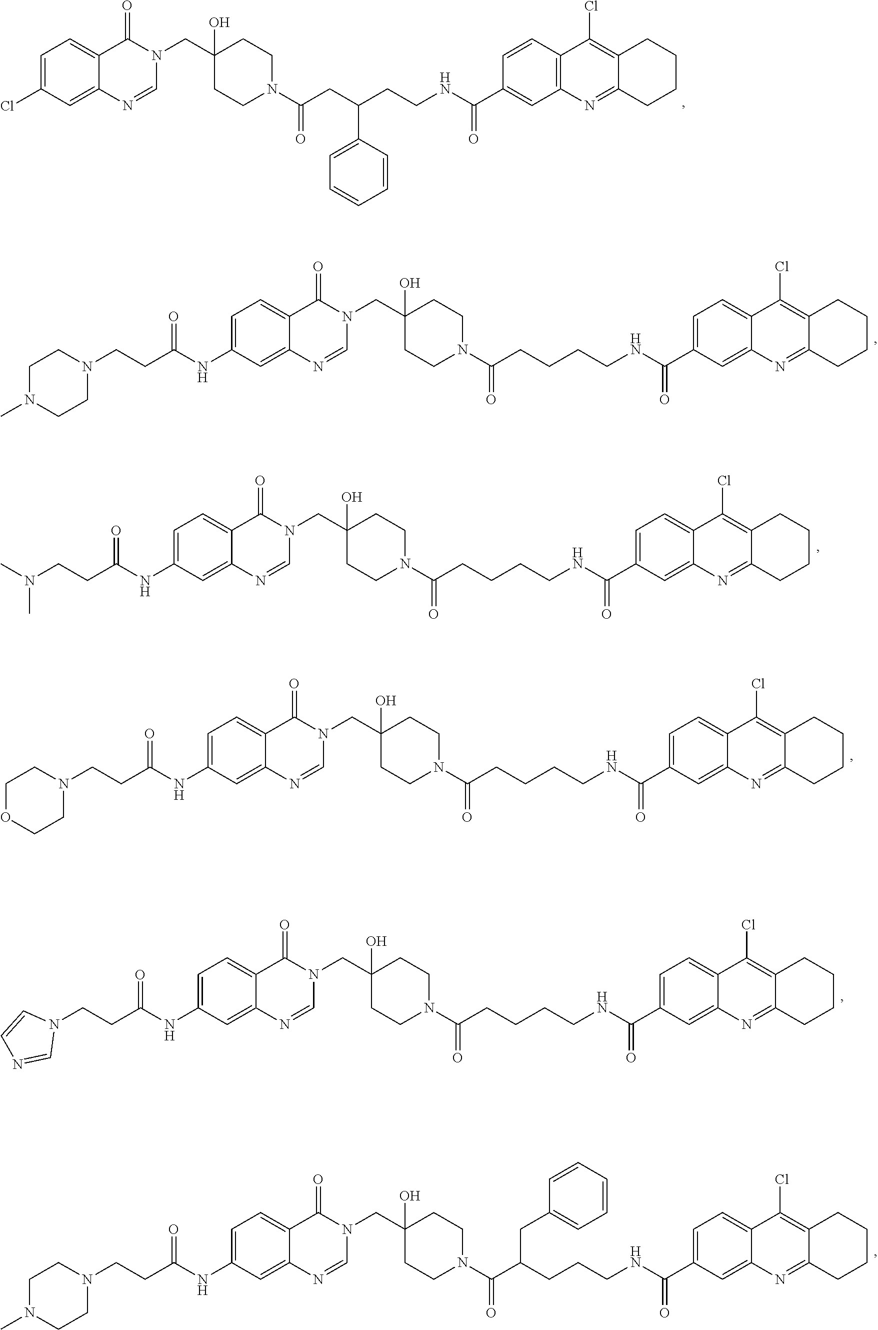

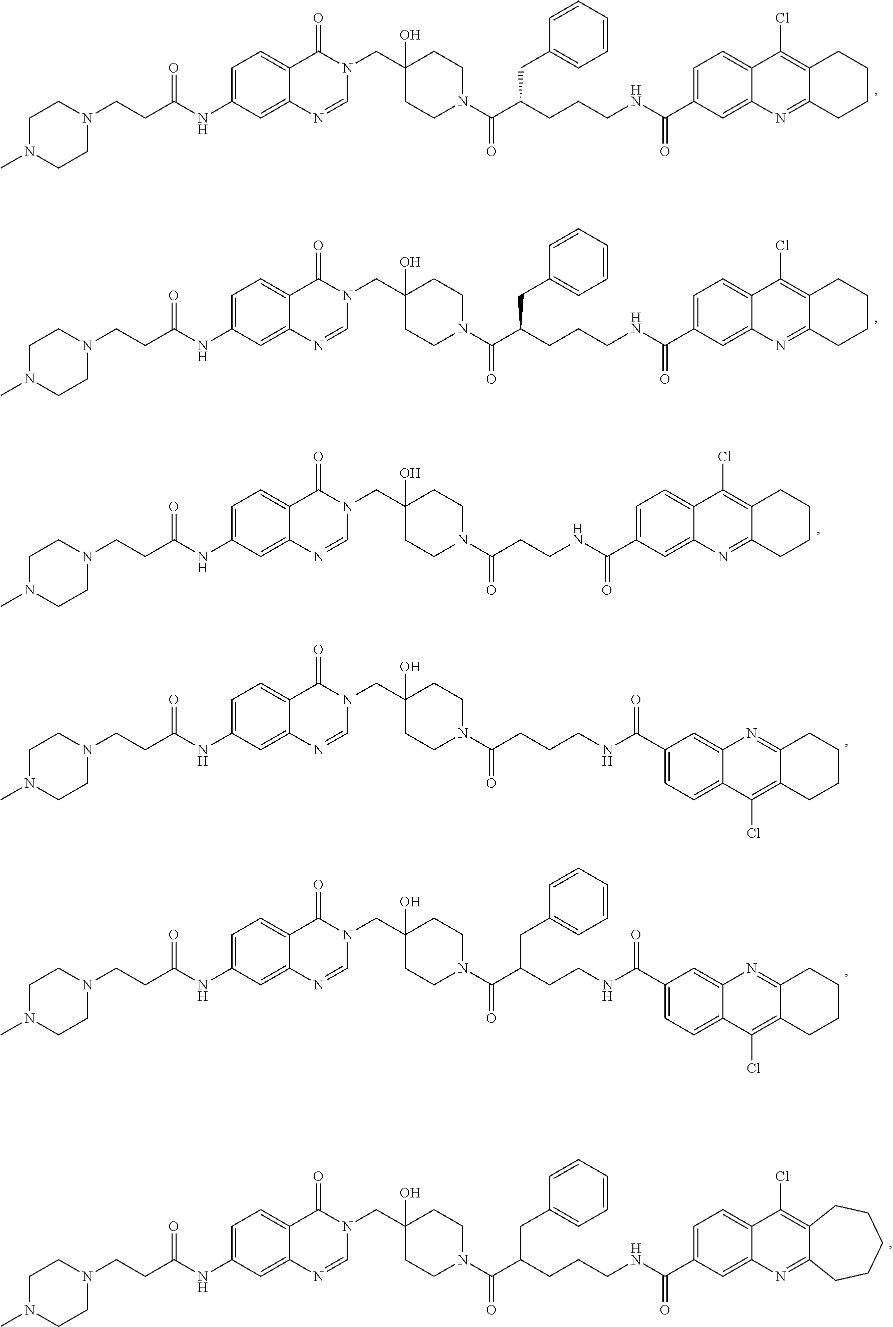

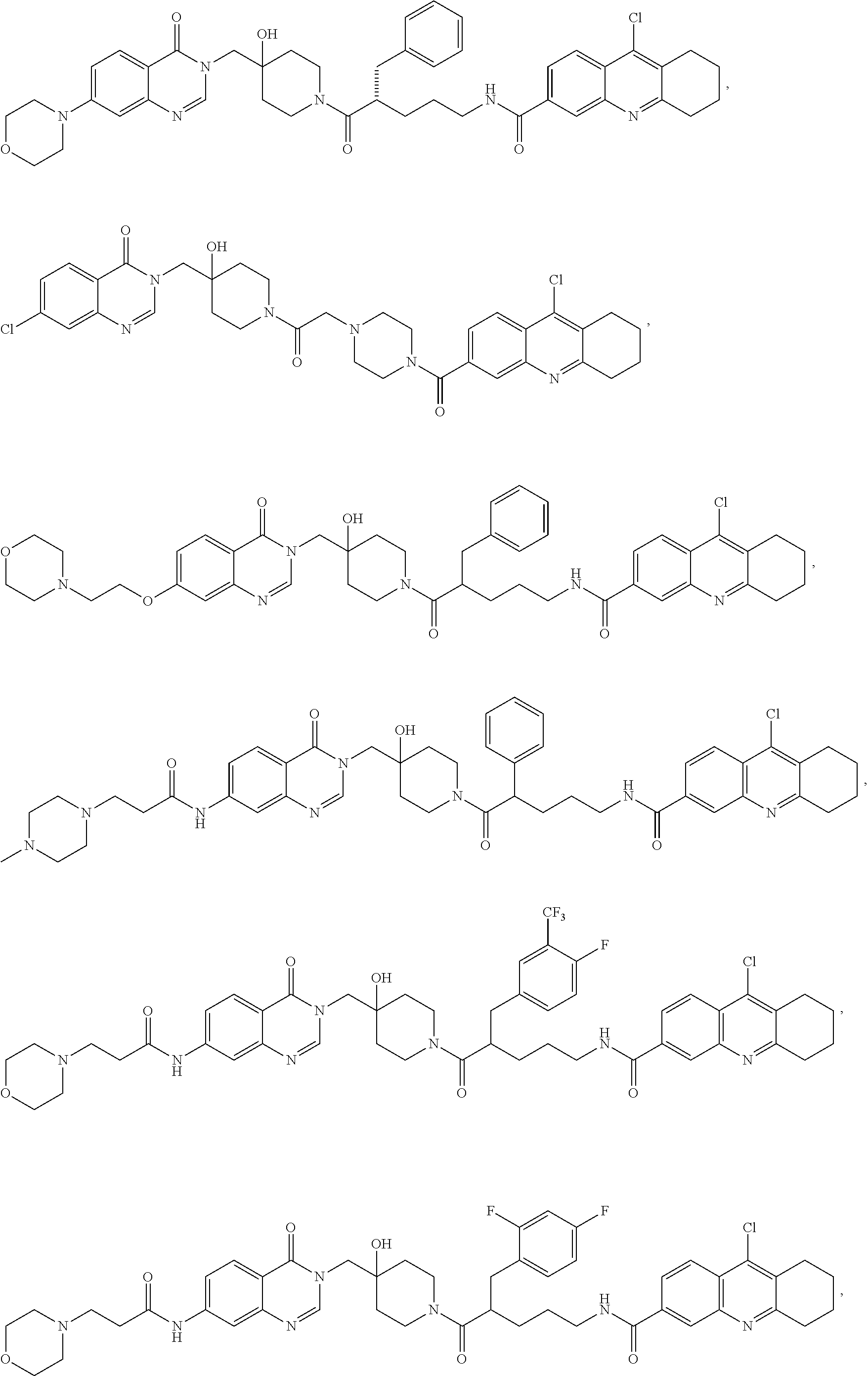

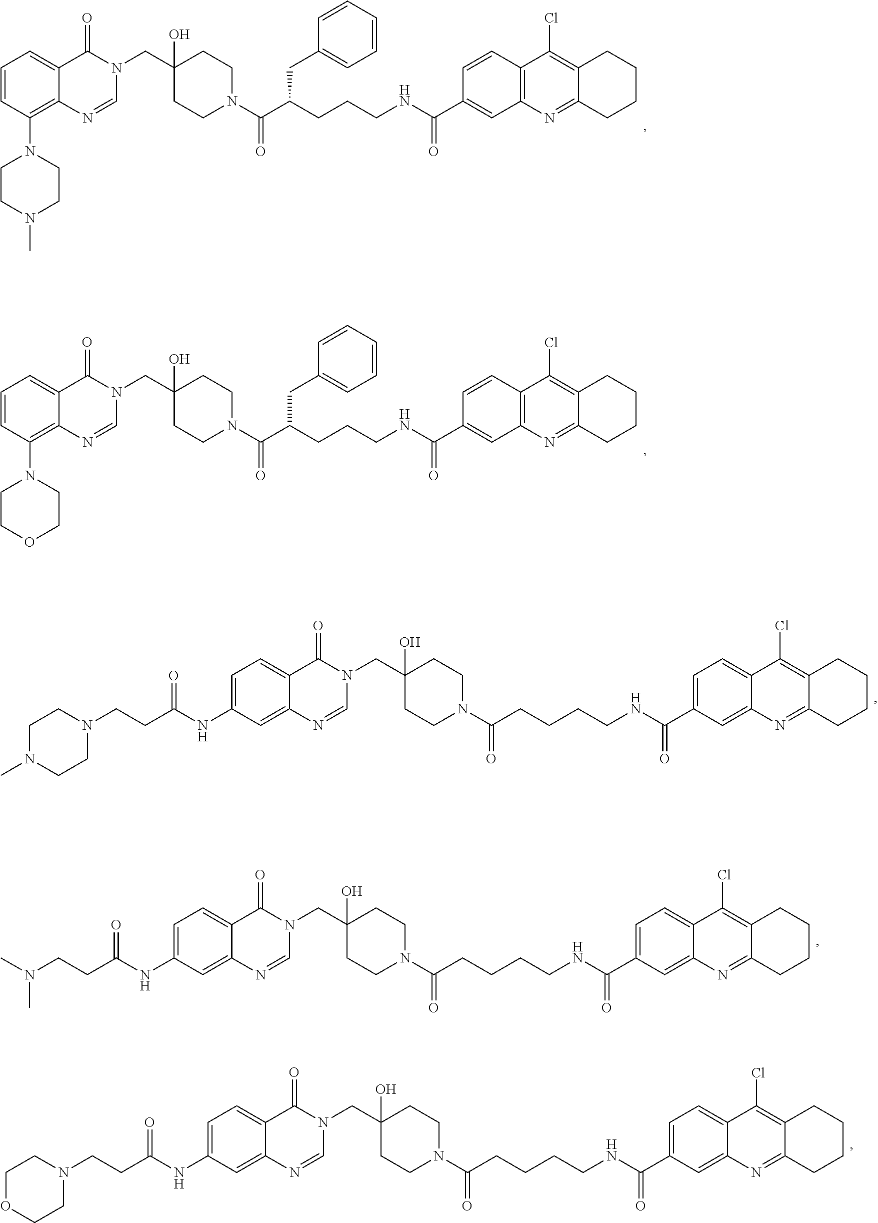 [SMILES: CN1CCN(c2cccc3c(=O)n(CC4(O)CCN(C(=O)[C@@H](CCCNC(=O)c5ccc6c(Cl)c7c(nc6c5)CCCC7)Cc5ccccc5)CC4)cnc23)CC1 ; O=C(NCCC[C@@H](Cc1ccccc1)C(=O)N1CCC(O)(Cn2cnc3c(N4CCOCC4)cccc3c2=O)CC1)c1ccc2c(Cl)c3c(nc2c1)CCCC3 ; CN1CCN(CCC(=O)Nc2ccc3c(=O)n(CC4(O)CCN(C(=O)CCCCNC(=O)c5ccc6c(Cl)c7c(nc6c5)CCCC7)CC4)cnc3c2)CC1 ; CN(C)CCC(=O)Nc1ccc2c(=O)n(CC3(O)CCN(C(=O)CCCCNC(=O)c4ccc5c(Cl)c6c(nc5c4)CCCC6)CC3)cnc2c1 ; O=C(CCN1CCOCC1)Nc1ccc2c(=O)n(CC3(O)CCN(C(=O)CCCCNC(=O)c4ccc5c(Cl)c6c(nc5c4)CCCC6)CC3)cnc2c1]

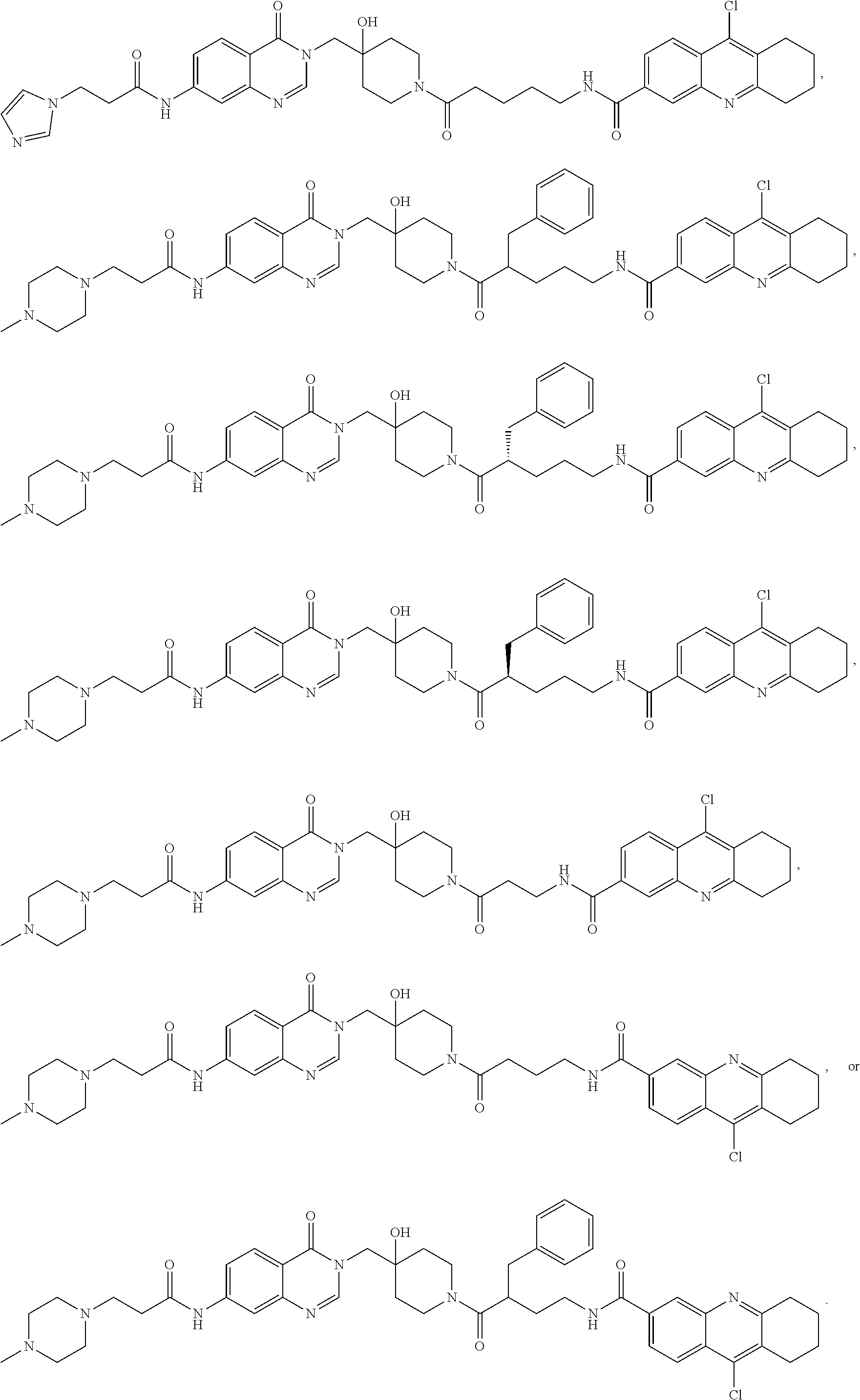

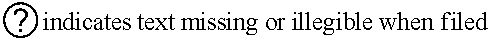

2. The compound of claim 1, wherein Q is

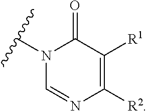

3. The compound of claim 1, wherein Q is

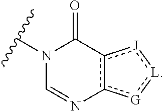

4. The compound of claim 1, wherein Q is

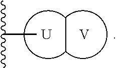

5. The compound of any one of claims 1-4, wherein L¹ is

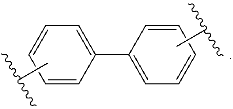

6. The compound of any one of claims 1-4, wherein L¹ is 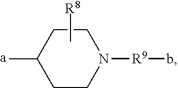
wherein a is the point of attachment to the carbonyl group and b is the point of attachment to L².

7. The compound of any one of claims 1-4, wherein L¹ is 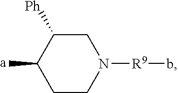
wherein a is the point of attachment to the carbonyl group and b is the point of attachment to L².

8. The compound of any one of claims 1-4, wherein L¹ is 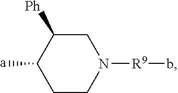
wherein a is the point of attachment to the carbonyl group and b is the point of attachment to L².

9. The compound of any one of claims 6-8, wherein R⁹ is a C₂₋₄ alkylene.

10. The compound of any one of claims 6-8, wherein R⁹ is a C₆₋₁₀ arylene.

11. The compound of any one of claims 1-10, wherein L² is —NH—.

12. The compound of any one of claims 1-11, wherein L³ is —S(═O)₂—.

13. The compound of any one of claims 1-11, wherein L³ is —C(═O)—.

14. The compound of any one of claims 1-13, wherein [symbol] is H and L⁴ is a C₁₋₃ alkyl or a C₃₋₄ alkenyl.

15. The compound of any one of claims 1-14, wherein L⁴ is C₃₋₄ alkenyl.

16. The compound of any one of claims 1-13, wherein [symbol] is

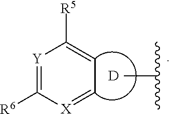

17. The compound of claim 16, wherein [symbol] is a 5-6 membered heterocyclyl or a 5-6 membered heteroaryl.

18. The compound of any one of claims 1-13, wherein [symbol] is

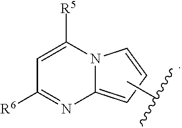

19. The compound of claim 1, wherein the compound of Formula (I) is a compound of Formula (Ia): 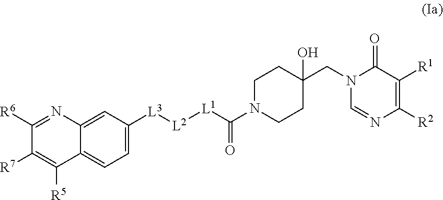
or a pharmaceutically acceptable salt thereof.

20. The compound of claim 19, wherein L² is —NH—.

21. The compound of claim 19, wherein L² is

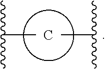

22. The compound of any one of claims 19-21, wherein L³ is —C(═O)—.

23. The compound of claim 1, wherein the compound of Formula (I) is a compound of Formula (Ib): 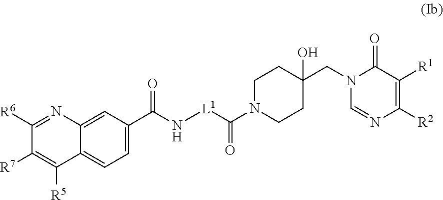
or a pharmaceutically acceptable salt thereof.

24. The compound of any one of claims 1-23, wherein R⁵ is Hal.

25. The compound of claim 24, wherein R⁵ is C₁.

26. The compound of any one of claims 1-23, wherein R is —OH.

27. The compound of any one of claims 1-23, wherein R is Me.

28. The compound of any one of claims 1-27, wherein R⁷ is H.

29. The compound of any one of claims 1-28, wherein R⁶ is H.

30. The compound of any one of claims 1-28, wherein R⁶ is F, —NH₂, or —OH, and wherein —NH₂ or —OH is optionally substituted with a C₁₋₃ alkyl;

31. The compound of any one of claims 1-28, wherein R⁶ is a C₁₋₃ alkyl, and wherein the C₁₋₃ alkyl is optionally substituted with one or more Hal or —NH₂.

32. The compound of any one of claims 1-28, wherein R⁶ is a C₆₋₁₀ aryl, and wherein the C₆₋₁₀ aryl is optionally substituted with one or more Hal, —NH₂, —CN, —CF₃, a C₁₋₃ alkyl, C₁₋₃ alkyl(NH₂), or C₁₋₃ alkyl(CF₃).

33. The compound of any one of claims 1-28, wherein R⁶ is a 5-6 membered heteroaryl.

34. The compound of any one of claims 1-28, wherein R⁶ is a C₃₋₅ cycloalkyl, and wherein the C₃₋₅ cycloalkyl is optionally substituted with one or more Hal.

35. The compound of any one of claims 1-28, wherein R⁷ and R⁶ taken together with the carbon atoms to which they are attached form a C₅ cycloalkyl or a 5-6 membered heterocyclyl, and wherein the C₅ cycloalkyl or the 5-6 membered heterocyclyl is optionally substituted with one or more C₁₋₃ alkyl, oxo, or —C(═O)C₁₋₃ alkyl.

36. The compound of any one of claims 1-28, wherein R⁷ and R⁶ taken together with the carbon atoms to which they are attached form a 5-6 membered heterocyclyl, and wherein the 5-6 membered heterocyclyl is optionally substituted with one or more C₁₋₃ alkyl, oxo, or —C(═O)C₁₋₃ alkyl.

37. The compound of any one of claims 1-28, wherein R⁷ and R⁶ taken together with the carbon atoms to which they are attached form a C₅ cycloalkyl.

38. The compound of any one of claims 1-4 and 11-37, wherein L¹ is a C₂₋₄ alkylene substituted with one or more —C₀₋₂ alkyl(C₆ aryl).

39. The compound of claim 38, wherein the one or more —C₀₋₂alkyl(C₆ aryl) is substituted with one or more Hal, —NH₂, —CN, —CF₃, or C₁₋₃ alkyl.

40. The compound of any one of claims 1-4 and 11-37, wherein L¹ is a C₂₋₄ alkylene substituted with —CH₂Ph.

41. The compound of any one of claims 1-4 and 11-37, wherein L¹ is 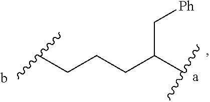
wherein a is the point of attachment to the carbonyl group and b is the point of attachment to L².

42. The compound of any one of claims 1-4 and 11-37, wherein L¹ is 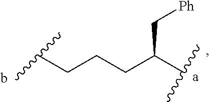
wherein a is the point of attachment to the carbonyl group and b is the point of attachment to L².

43. The compound of any one of claims 1-4 and 11-37, wherein L¹ is 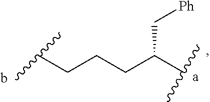
wherein a is the point of attachment to the carbonyl group and b is the point of attachment to L².

44. The compound of any one of claims 1-4 and 11-37, wherein L¹ is

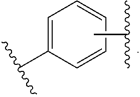

45. The compound of any one of claims 1-4 and 11-37, wherein L¹ is 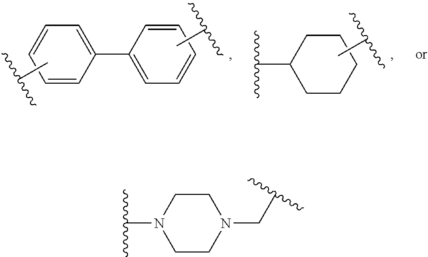
in either direction.

46. The compound of any one of claims 1-4 and 11-37, wherein L¹ is 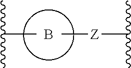
in either direction.

47. The compound of any one of claims 1-4 and 11-37, wherein L¹ is 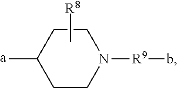
wherein a is the point of attachment to the carbonyl group and b is the point of attachment to L².

48. The compound of any one of claims 1-4 and 11-37, wherein L¹ is 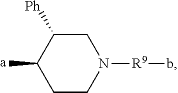
wherein a is the bond to the carbonyl group and b is the bond to L².

49. The compound of any one of claims 1-4 and 11-37, wherein L¹ is 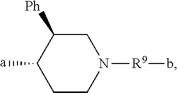
wherein a is the point of attachment to the carbonyl group and b is the point of attachment to L².

50. The compound of any one of claims 47-49, wherein R⁹ is a C₂₋₄ alkylene.

51. The compound of any one of claims 47-49, wherein R⁹ is a C₆₋₁₀ arylene.

52. The compound of claim 1 or 2, wherein R⁹ is

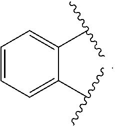 [SMILES: *c1ccccc1*]

53. The compound of any one of claims 1-10, wherein L² is a bond.

54. The compound of any one of claims 1-10, wherein L² is —NH—.

55. The compound of any one of claims 1-10, wherein L²

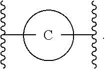

56. The compound of any one of claims 1-11, wherein L³ is —C(═O)—.

57. The compound of any one of claims 1-56, wherein R¹ is H.

58. The compound of any one of claims 1-57, wherein R² is a —NHC₁₋₃ alkylene(C₅₋₇ heterocyclyl).

59. The compound of any one of claims 1-56, wherein R¹ and R² taken together with the carbon atoms to which they are attached form a C₆₋₁₀ aryl.

60. The compound of claim 59, wherein the C₆₋₁₀ aryl is substituted with one or more —NHC(═O)C₁₋₃ alkylene(NR³R⁴).

61. The compound of claim 59, wherein the C₆₋₁₀ aryl is substituted with one or more —NHC(═O)C₂alkylene(NMe₂).

62. The compound of claim 59, wherein the C₆₋₁₀ aryl is substituted with one or more —OC₁₋₃ alkylene(NR³R⁴)

63. The compound of claim 60 or 62, wherein each R³ and R⁴ is independently a C₁₋₃ alkyl.

64. The compound of claim 63, wherein each R³ and R⁴ is Me.

65. The compound of claim 60 or 62, wherein R³ and R⁴ together with the nitrogen atom to which they are attached form a 5-7 membered heterocyclyl.

66. The compound of claim 65, wherein the 5-7 membered heterocyclyl is substituted with one or more C₁₋₃ alkyl.

67. The compound of claim 60 or 62, wherein R³ and R⁴ together with the nitrogen atom to which they are attached form a 5-7 membered heteroaryl.

68. The compound of any one of claims 1-56, wherein R¹ and R² taken together with the carbon atoms to which they are attached form a 5-6 membered heteroaryl.

69. The compound of claim 68, wherein the 5-6 membered heteroaryl is substituted with a one or more C₆₋₁₀ aryl.

70. The compound of claim 68, wherein the C₆₋₁₀ aryl is substituted with one or more Hal, —NH₂, —CN, —CF₃, or C₁₋₃ alkyl.

71. The compound of claim 1, wherein the compound of Formula (I) is a compound of Formula (Ic) or a compound of Formula (Id): 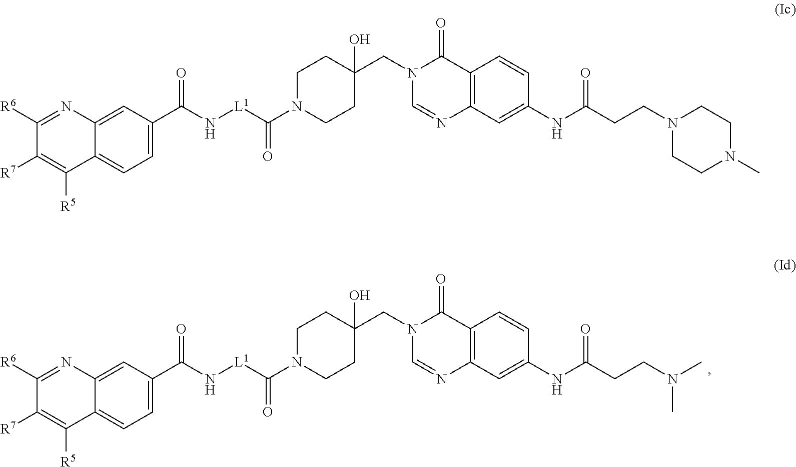
or a pharmaceutically acceptable salt thereof.

72. The compound of any one of claims 1-71, wherein the compound is not

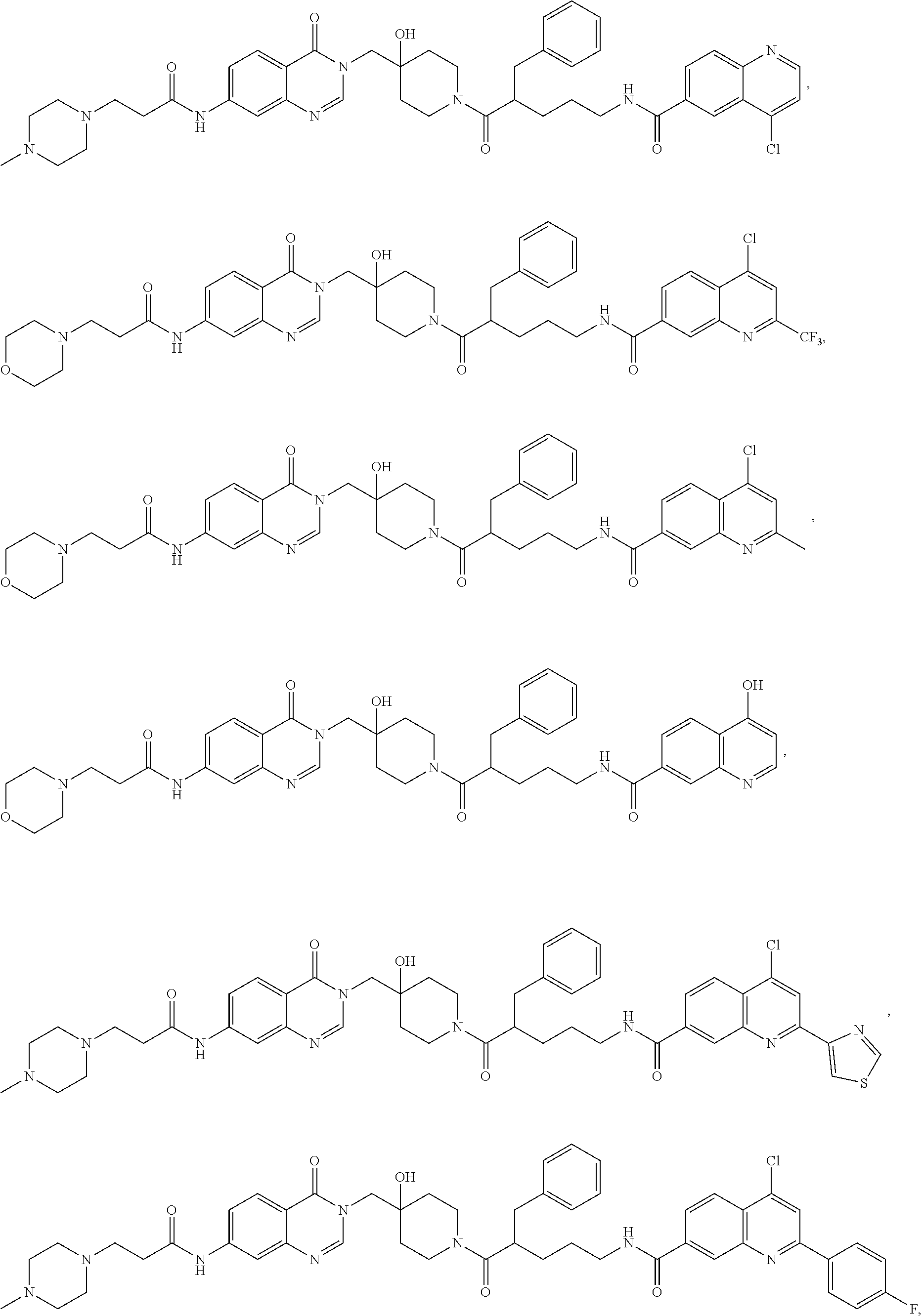

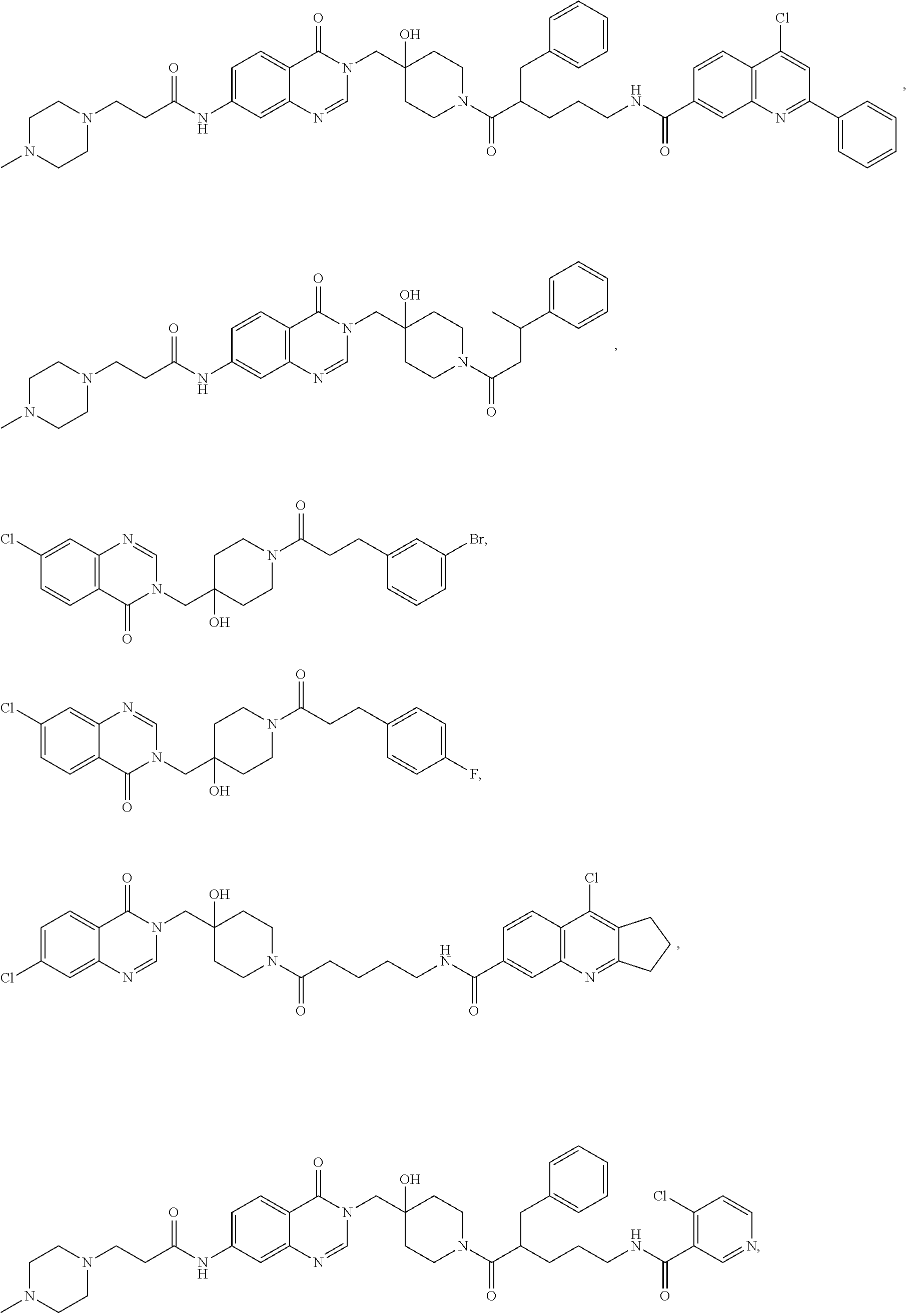 [SMILES: CN1CCN(CCC(=O)Nc2ccc3c(=O)n(CC4(O)CCN(C(=O)C(CCCNC(=O)c5ccc6c(Cl)cc(-c7ccccc7)nc6c5)Cc5ccccc5)CC4)cnc3c2)CC1 ; CC(CC(=O)N1CCC(O)(Cn2cnc3cc(NC(=O)CCN4CCN(C)CC4)ccc3c2=O)CC1)c1ccccc1 ; O=C(CCc1cccc(Br)c1)N1CCC(O)(Cn2cnc3cc(Cl)ccc3c2=O)CC1 ; O=C(CCc1ccc(F)cc1)N1CCC(O)(Cn2cnc3cc(Cl)ccc3c2=O)CC1 ; O=C(NCCCCC(=O)N1CCC(O)(Cn2cnc3cc(Cl)ccc3c2=O)CC1)c1ccc2c(Cl)c3c(nc2c1)CCC3 ; CN1CCN(CCC(=O)Nc2ccc3c(=O)n(CC4(O)CCN(C(=O)C(CCCNC(=O)c5cnccc5Cl)Cc5ccccc5)CC4)cnc3c2)CC1]

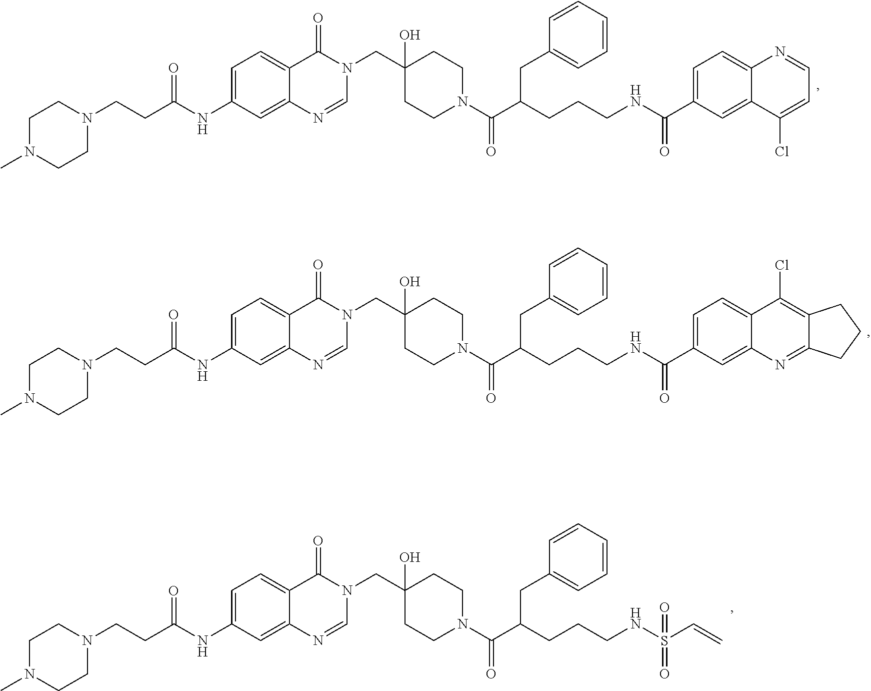 [SMILES: CN1CCN(CCC(=O)Nc2ccc3c(=O)n(CC4(O)CCN(C(=O)C(CCCNC(=O)c5ccc6nccc(Cl)c6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)c7c(nc6c5)CCC7)Cc5ccccc5)CC4)cnc3c2)CC1 ; C=CS(=O)(=O)NCCCC(Cc1ccccc1)C(=O)N1CCC(O)(Cn2cnc3cc(NC(=O)CCN4CCN(C)CC4)ccc3c2=O)CC1]

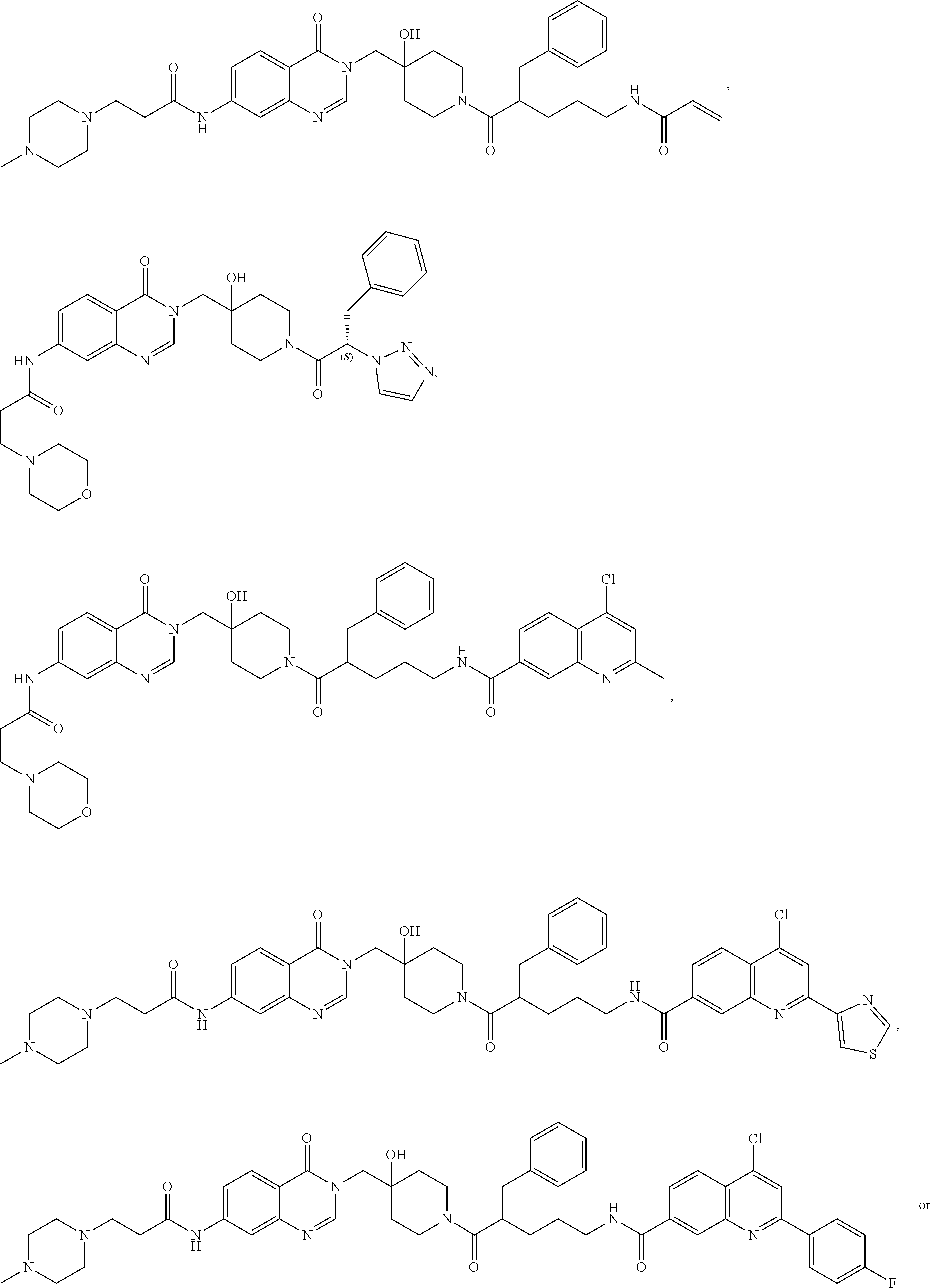

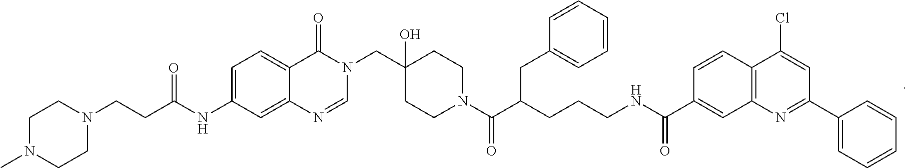 [SMILES: CN1CCN(CCC(=O)Nc2ccc3c(=O)n(CC4(O)CCN(C(=O)C(CCCNC(=O)c5ccc6c(Cl)cc(-c7ccccc7)nc6c5)Cc5ccccc5)CC4)cnc3c2)CC1]

73. The compound of claim 1 selected from the group consisting of 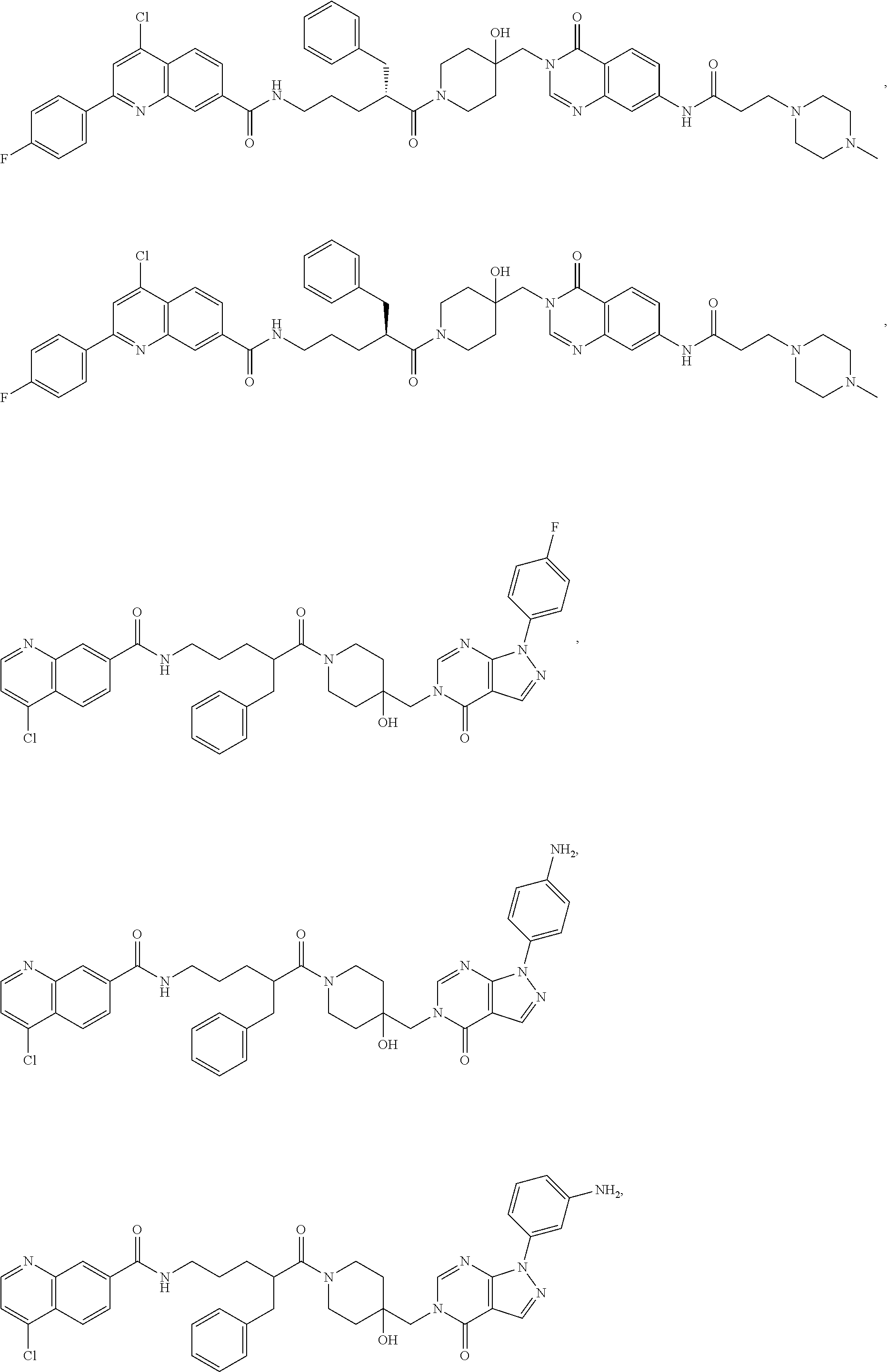 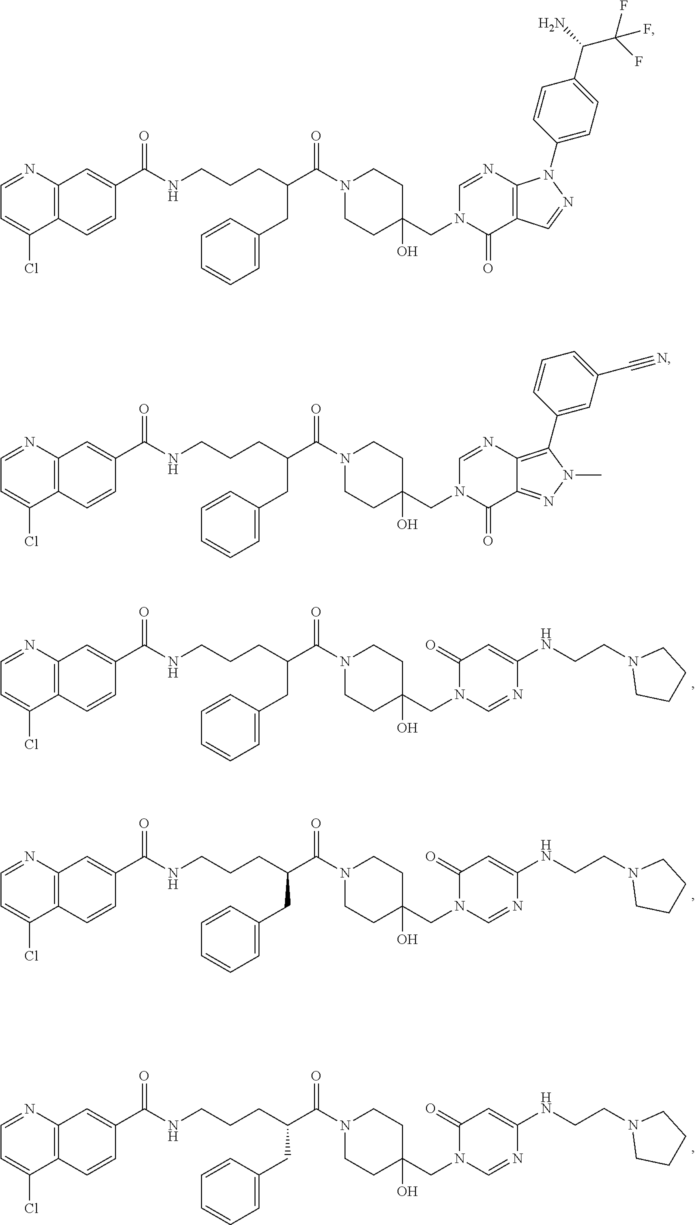 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F ; Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1 ; O=C(NCCCC(Cc1ccccc1)C(=O)N1CCC(O)(Cn2cnc(NCCN3CCCC3)cc2=O)CC1)c1ccc2c(Cl)ccnc2c1 ; O=C(NCCC[C@H](Cc1ccccc1)C(=O)N1CCC(O)(Cn2cnc(NCCN3CCCC3)cc2=O)CC1)c1ccc2c(Cl)ccnc2c1 ; O=C(NCCC[C@@H](Cc1ccccc1)C(=O)N1CCC(O)(Cn2cnc(NCCN3CCCC3)cc2=O)CC1)c1ccc2c(Cl)ccnc2c1] 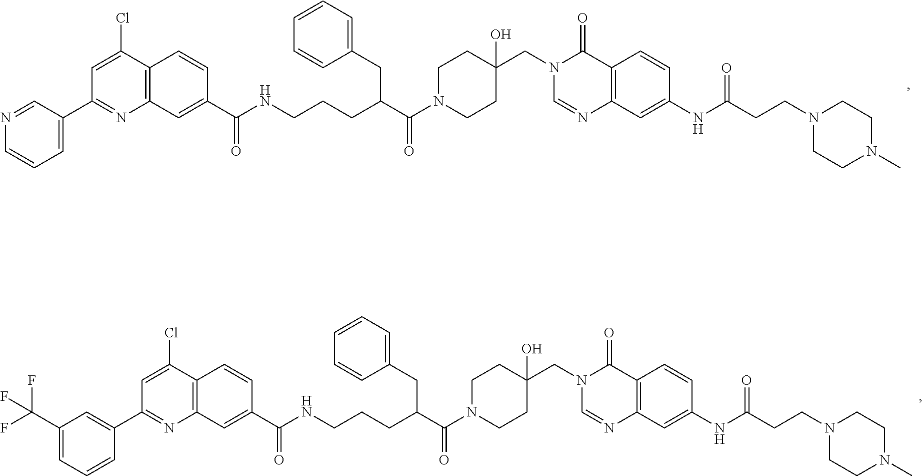 [SMILES: CN1CCN(CCC(=O)Nc2ccc3c(=O)n(CC4(O)CCN(C(=O)C(CCCNC(=O)c5ccc6c(Cl)cc(-c7cccnc7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7cccc(C(F)(F)F)c7)nc6c5)Cc5ccccc5)CC4)cnc3c2)CC1] 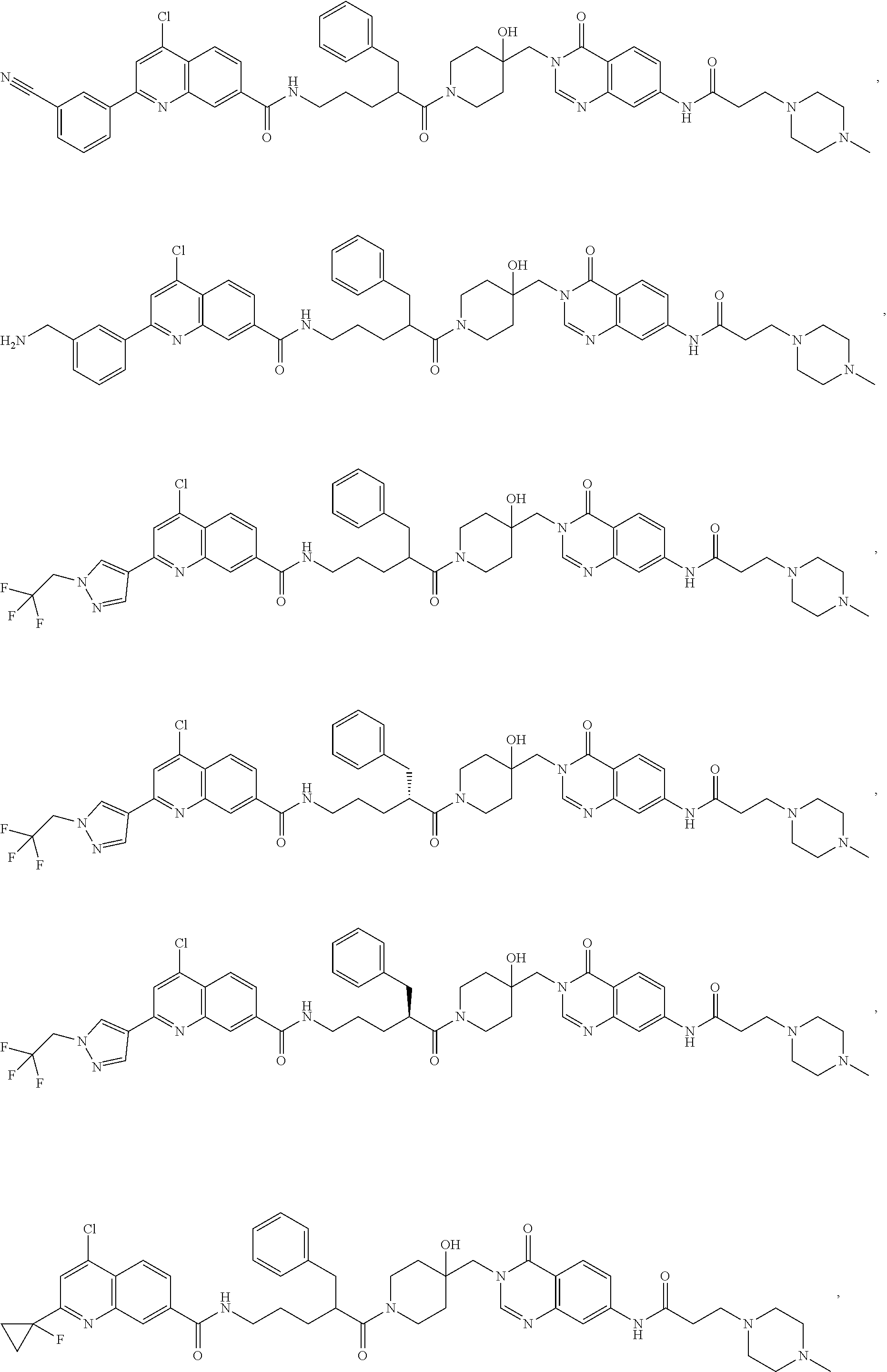 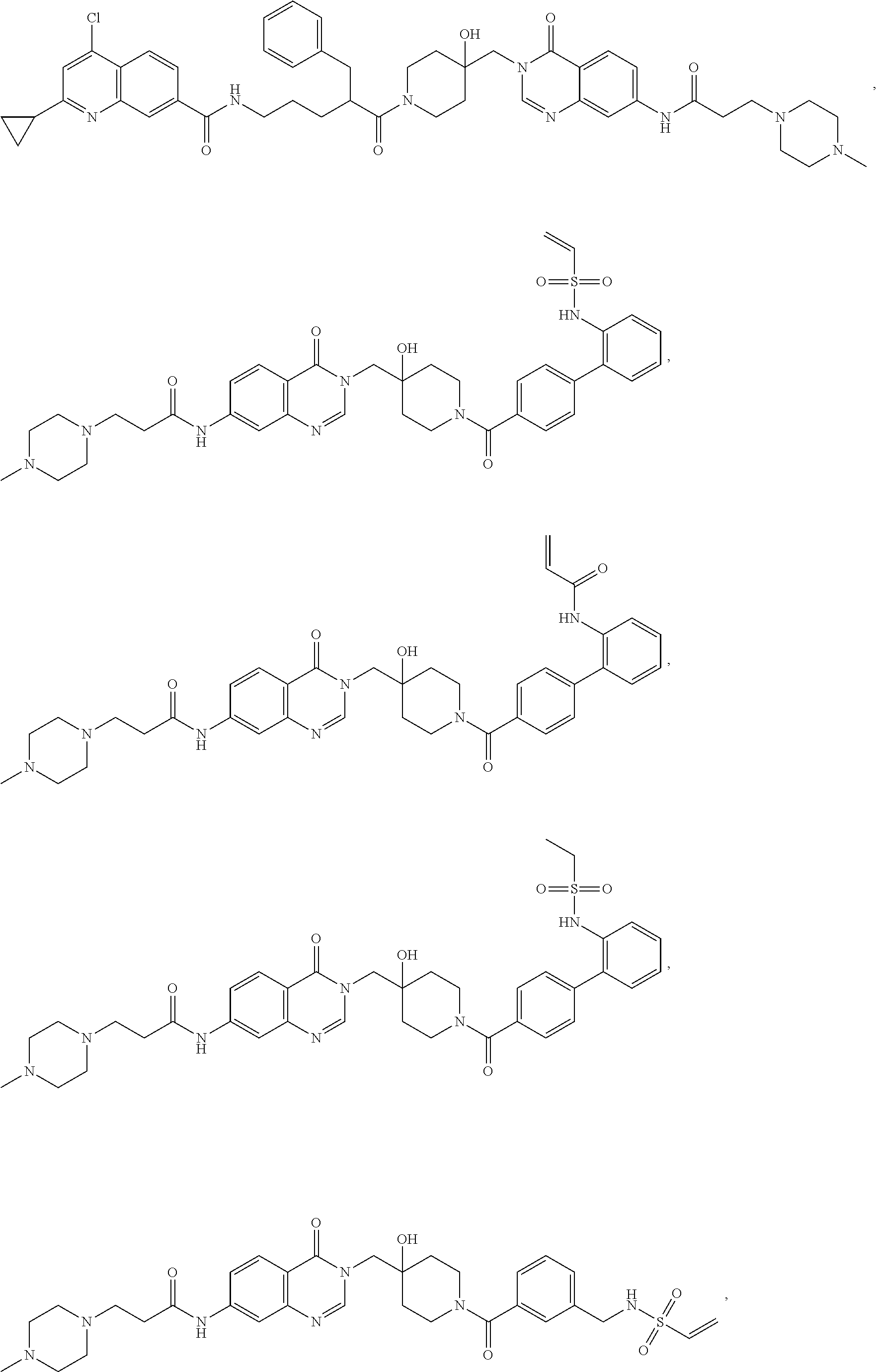 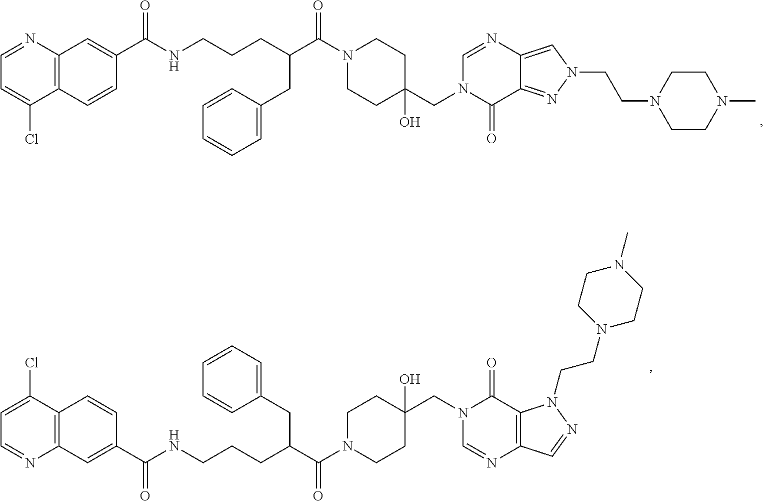 [SMILES: CN1CCN(CCn2cc3ncn(CC4(O)CCN(C(=O)C(CCCNC(=O)c5ccc6c(Cl)ccnc6c5)Cc5ccccc5)CC4)c(=O)c3n2)CC1 ; CN1CCN(CCn2ncc3ncn(CC4(O)CCN(C(=O)C(CCCNC(=O)c5ccc6c(Cl)ccnc6c5)Cc5ccccc5)CC4)c(=O)c32)CC1] 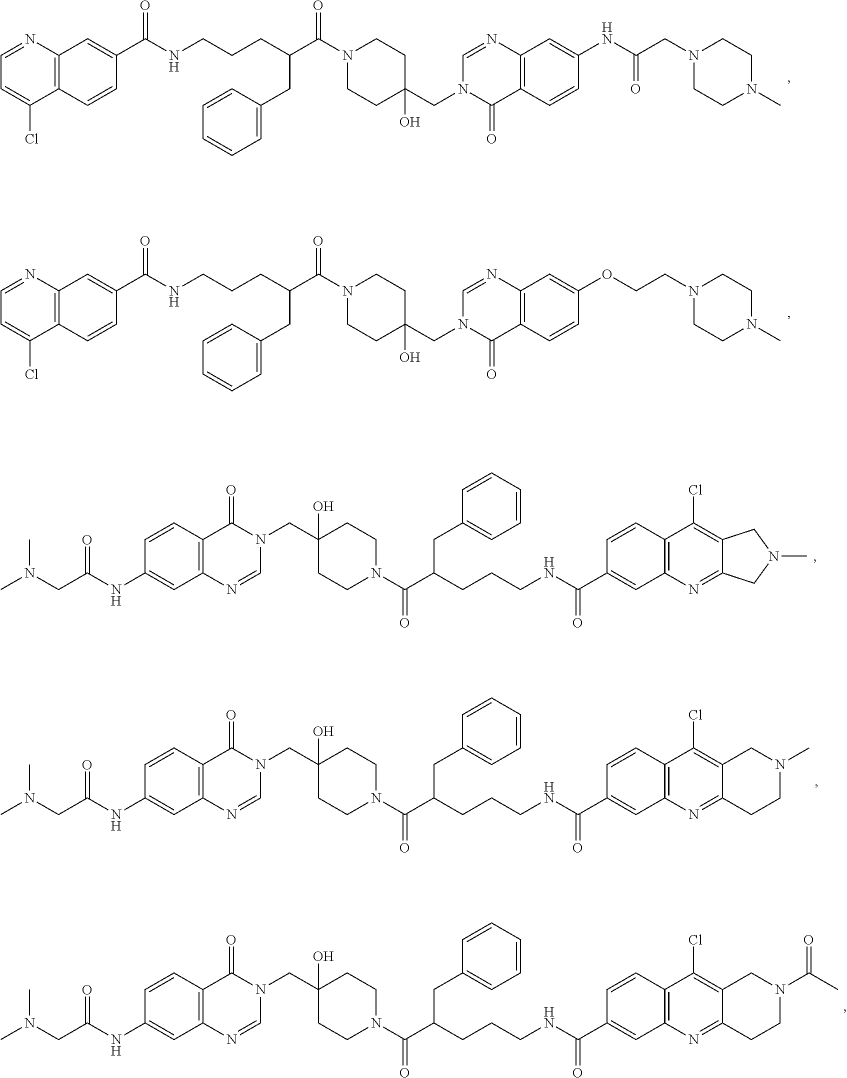 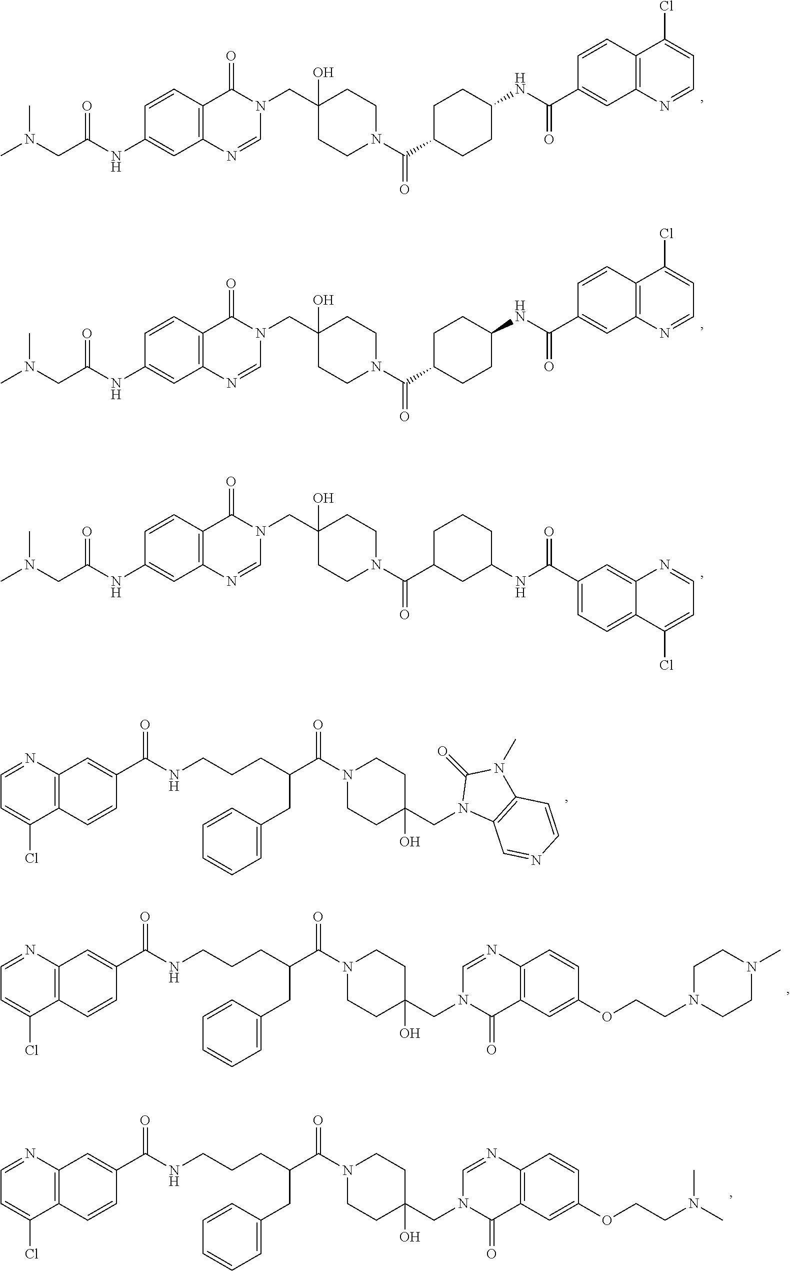 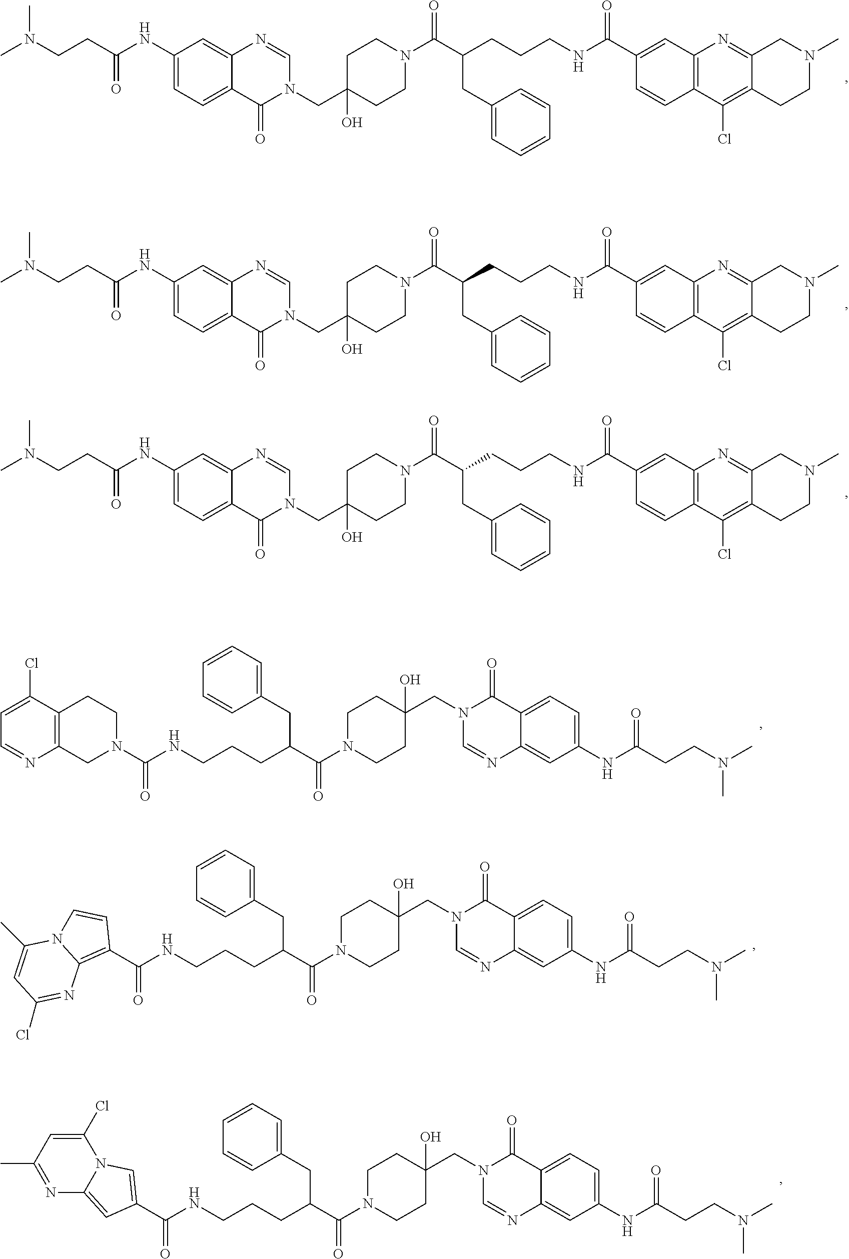 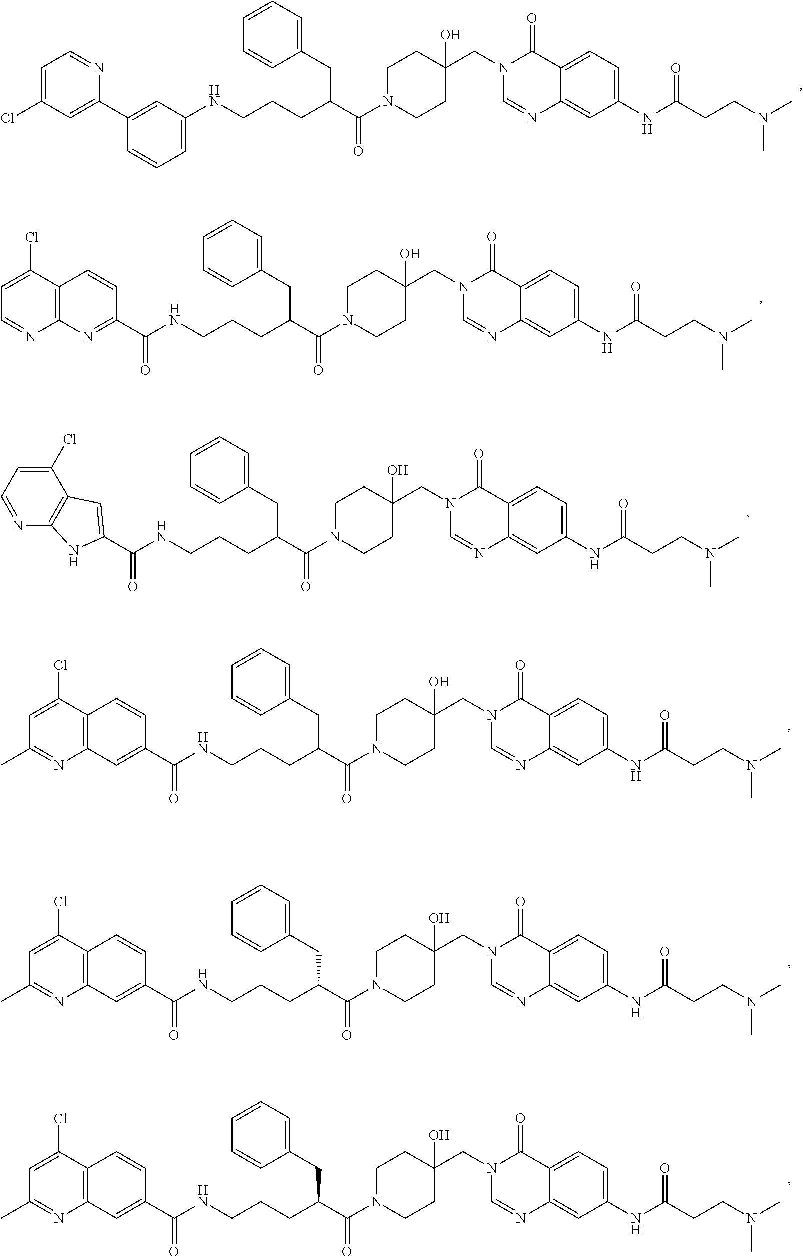 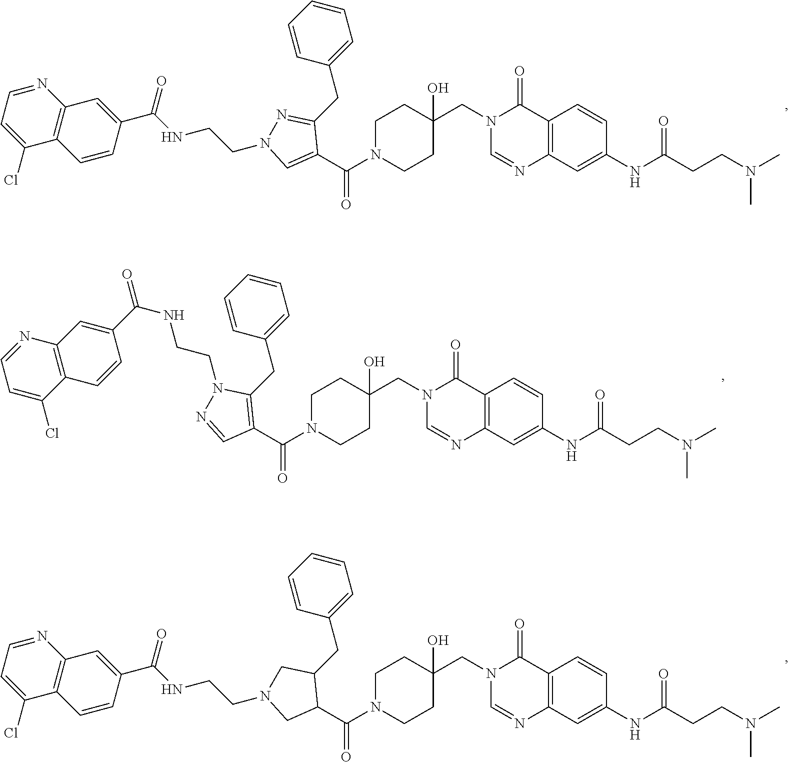 [SMILES: CN(C)CCC(=O)Nc1ccc2c(=O)n(CC3(O)CCN(C(=O)c4cn(CCNC(=O)c5ccc6c(Cl)ccnc6c5)nc4Cc4ccccc4)CC3)cnc2c1 ; CN(C)CCC(=O)Nc1ccc2c(=O)n(CC3(O)CCN(C(=O)c4cnn(CCNC(=O)c5ccc6c(Cl)ccnc6c5)c4Cc4ccccc4)CC3)cnc2c1 ; CN(C)CCC(=O)Nc1ccc2c(=O)n(CC3(O)CCN(C(=O)C4CN(CCNC(=O)c5ccc6c(Cl)ccnc6c5)CC4Cc4ccccc4)CC3)cnc2c1] 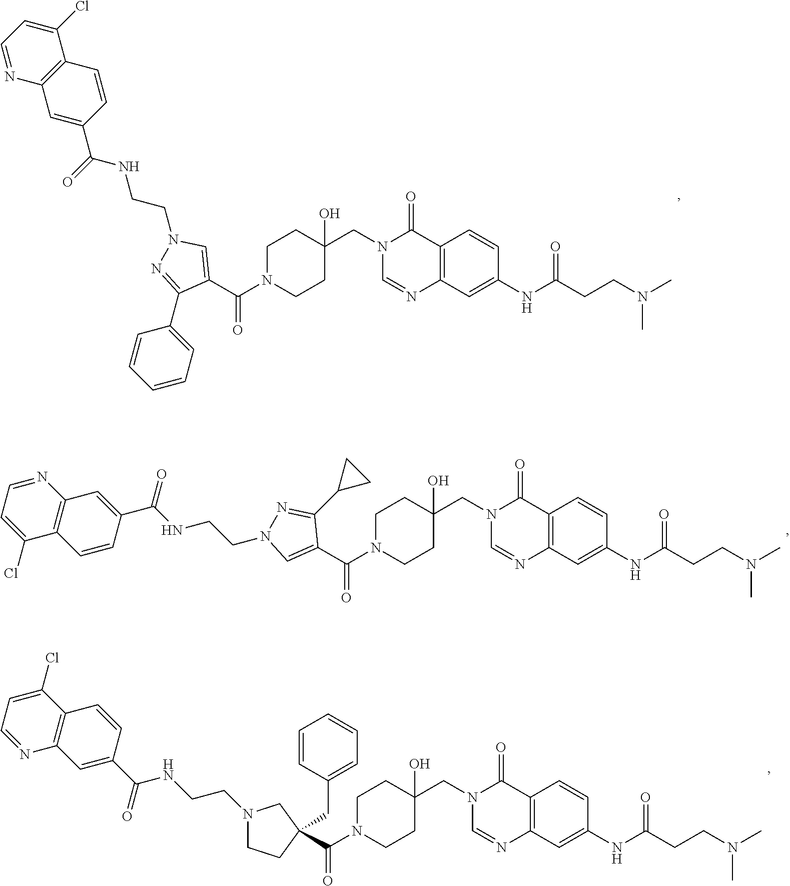 [SMILES: CN(C)CCC(=O)Nc1ccc2c(=O)n(CC3(O)CCN(C(=O)c4cn(CCNC(=O)c5ccc6c(Cl)ccnc6c5)nc4-c4ccccc4)CC3)cnc2c1 ; CN(C)CCC(=O)Nc1ccc2c(=O)n(CC3(O)CCN(C(=O)c4cn(CCNC(=O)c5ccc6c(Cl)ccnc6c5)nc4C4CC4)CC3)cnc2c1 ; CN(C)CCC(=O)Nc1ccc2c(=O)n(CC3(O)CCN(C(=O)[C@@]4(Cc5ccccc5)CCN(CCNC(=O)c5ccc6c(Cl)ccnc6c5)C4)CC3)cnc2c1] 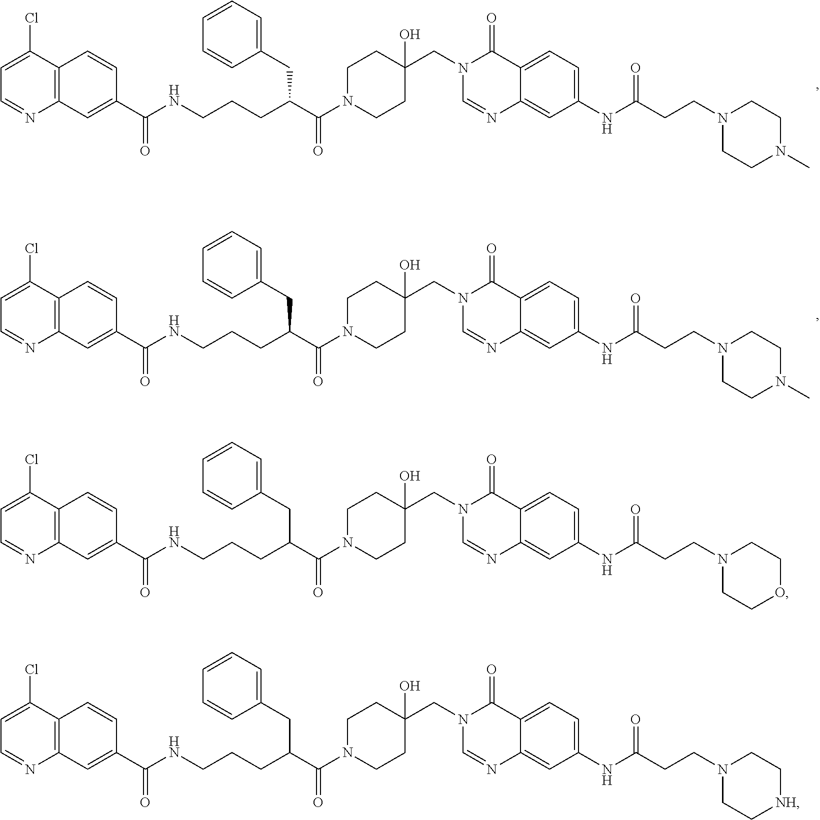 [SMILES: CN1CCN(CCC(=O)Nc2ccc3c(=O)n(CC4(O)CCN(C(=O)[C@H](CCCNC(=O)c5ccc6c(Cl)ccnc6c5)Cc5ccccc5)CC4)cnc3c2)CC1 ; CN1CCN(CCC(=O)Nc2ccc3c(=O)n(CC4(O)CCN(C(=O)[C@@H](CCCNC(=O)c5ccc6c(Cl)ccnc6c5)Cc5ccccc5)CC4)cnc3c2)CC1 ; O=C(CCN1CCOCC1)Nc1ccc2c(=O)n(CC3(O)CCN(C(=O)C(CCCNC(=O)c4ccc5c(Cl)ccnc5c4)Cc4ccccc4)CC3)cnc2c1 ; O=C(CCN1CCNCC1)Nc1ccc2c(=O)n(CC3(O)CCN(C(=O)C(CCCNC(=O)c4ccc5c(Cl)ccnc5c4)Cc4ccccc4)CC3)cnc2c1] 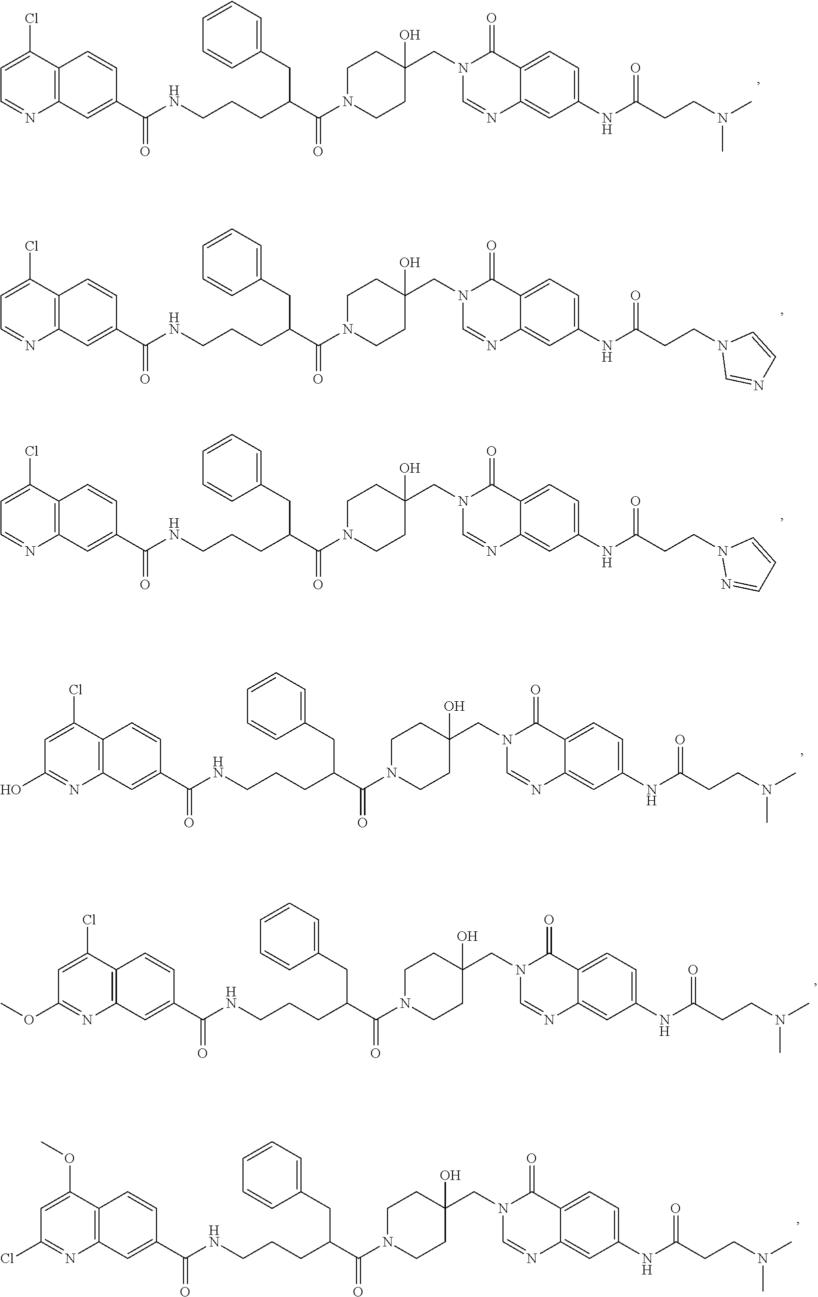 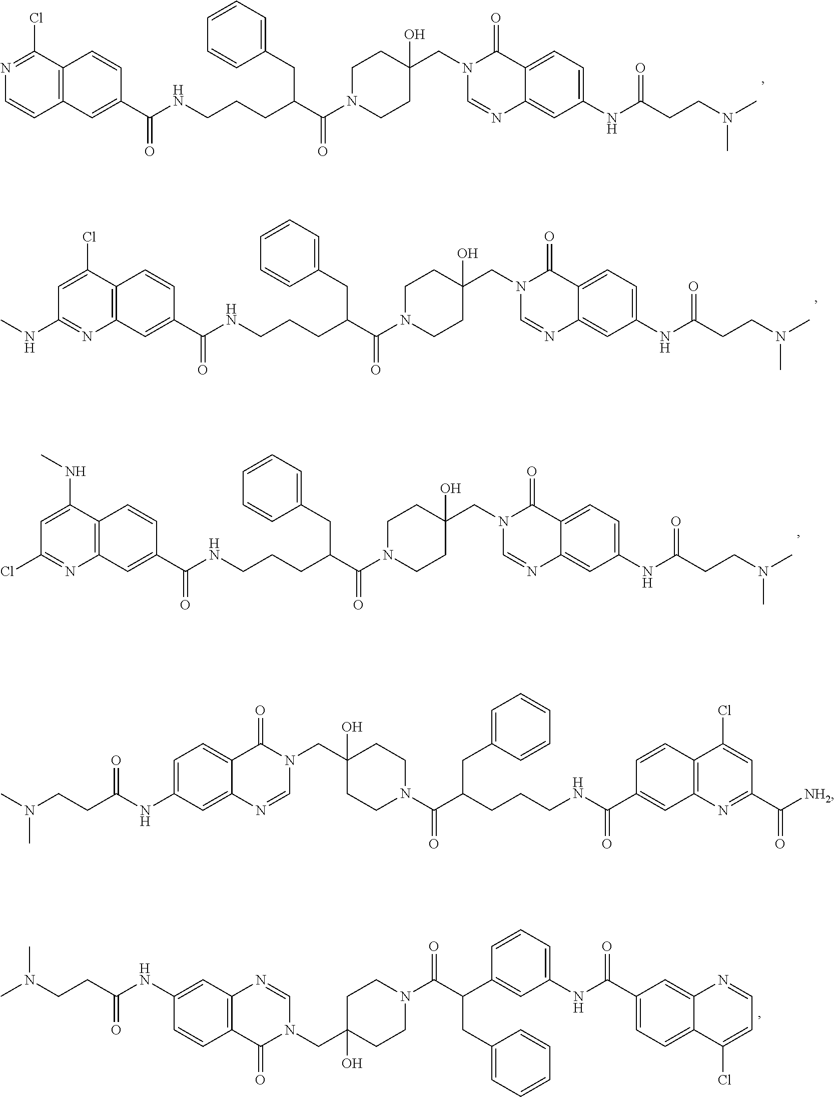 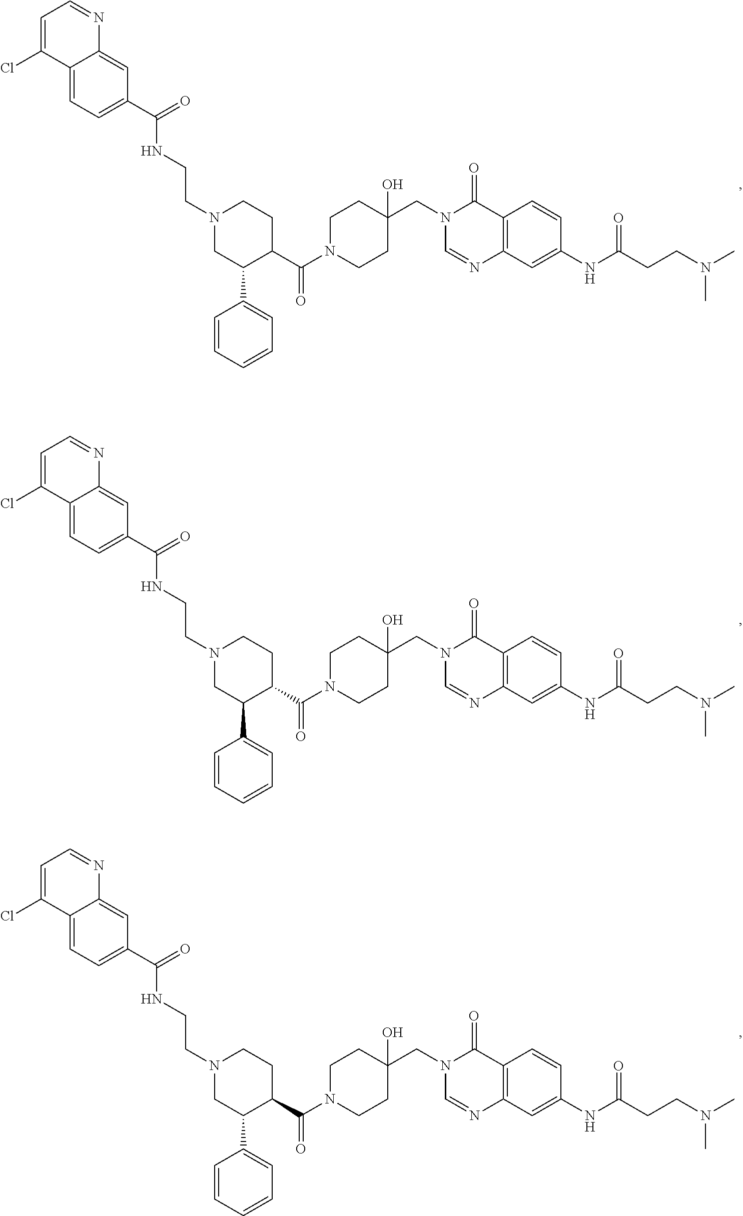 [SMILES: CN(C)CCC(=O)Nc1ccc2c(=O)n(CC3(O)CCN(C(=O)C4CCN(CCNC(=O)c5ccc6c(Cl)ccnc6c5)C[C@H]4c4ccccc4)CC3)cnc2c1 ; CN(C)CCC(=O)Nc1ccc2c(=O)n(CC3(O)CCN(C(=O)[C@H]4CCN(CCNC(=O)c5ccc6c(Cl)ccnc6c5)C[C@@H]4c4ccccc4)CC3)cnc2c1 ; CN(C)CCC(=O)Nc1ccc2c(=O)n(CC3(O)CCN(C(=O)[C@@H]4CCN(CCNC(=O)c5ccc6c(Cl)ccnc6c5)C[C@H]4c4ccccc4)CC3)cnc2c1] 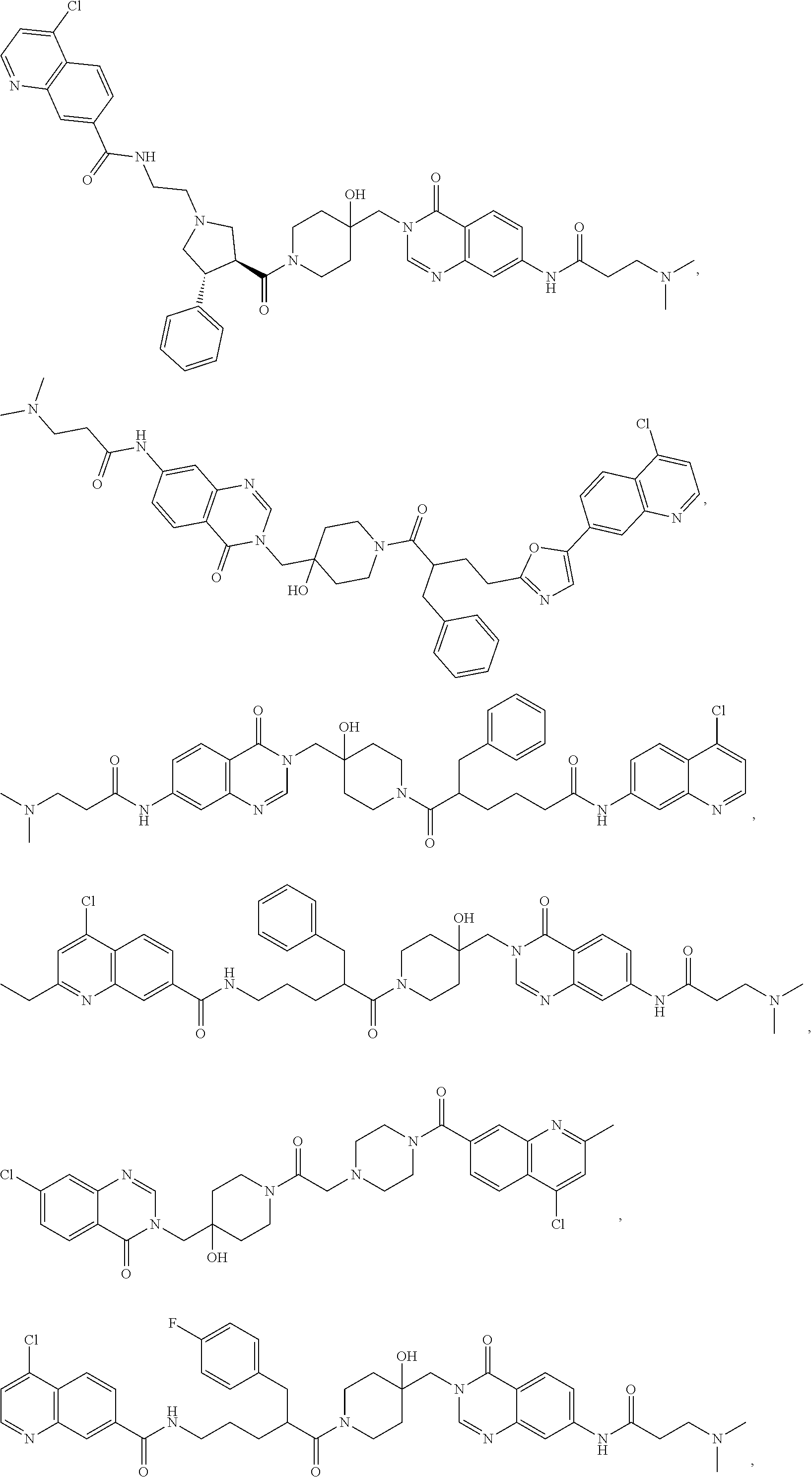 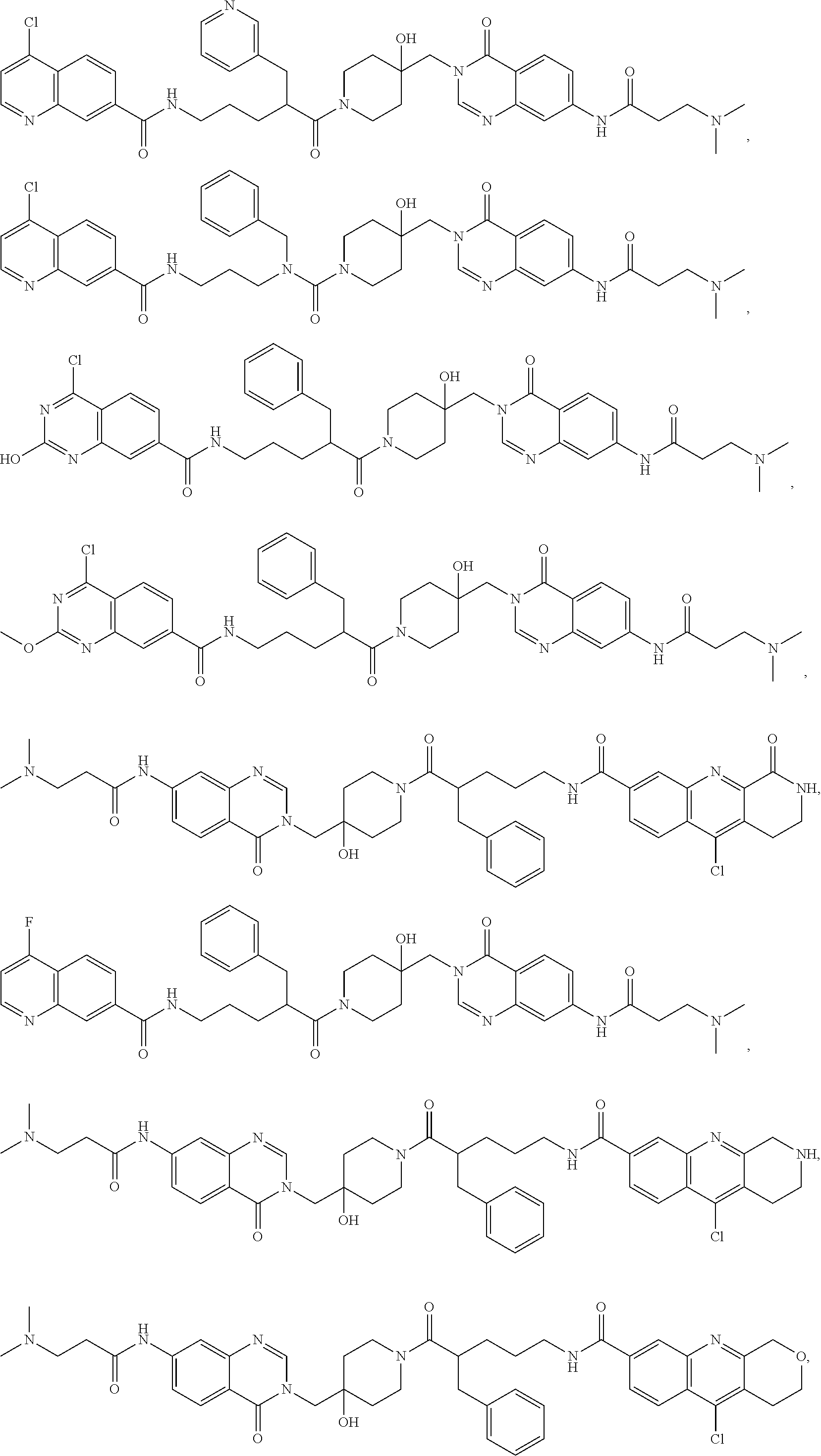 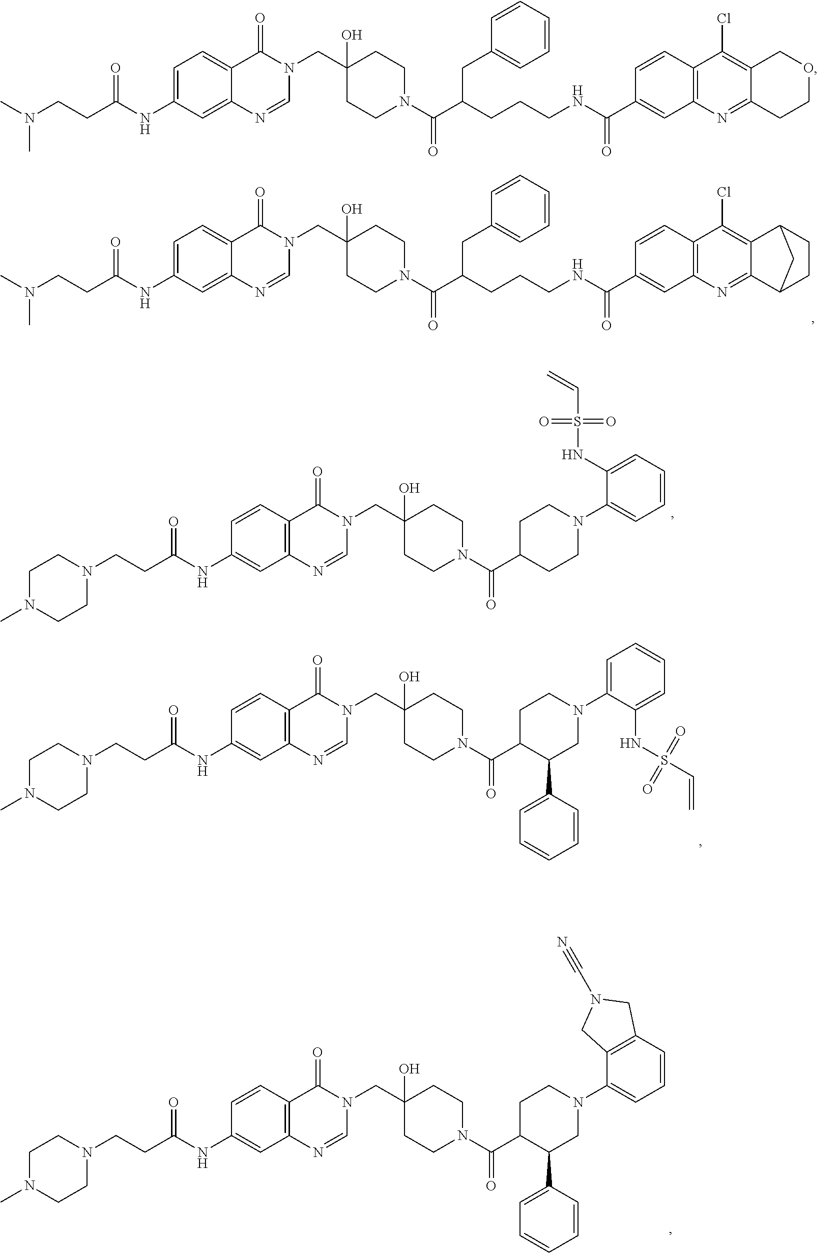 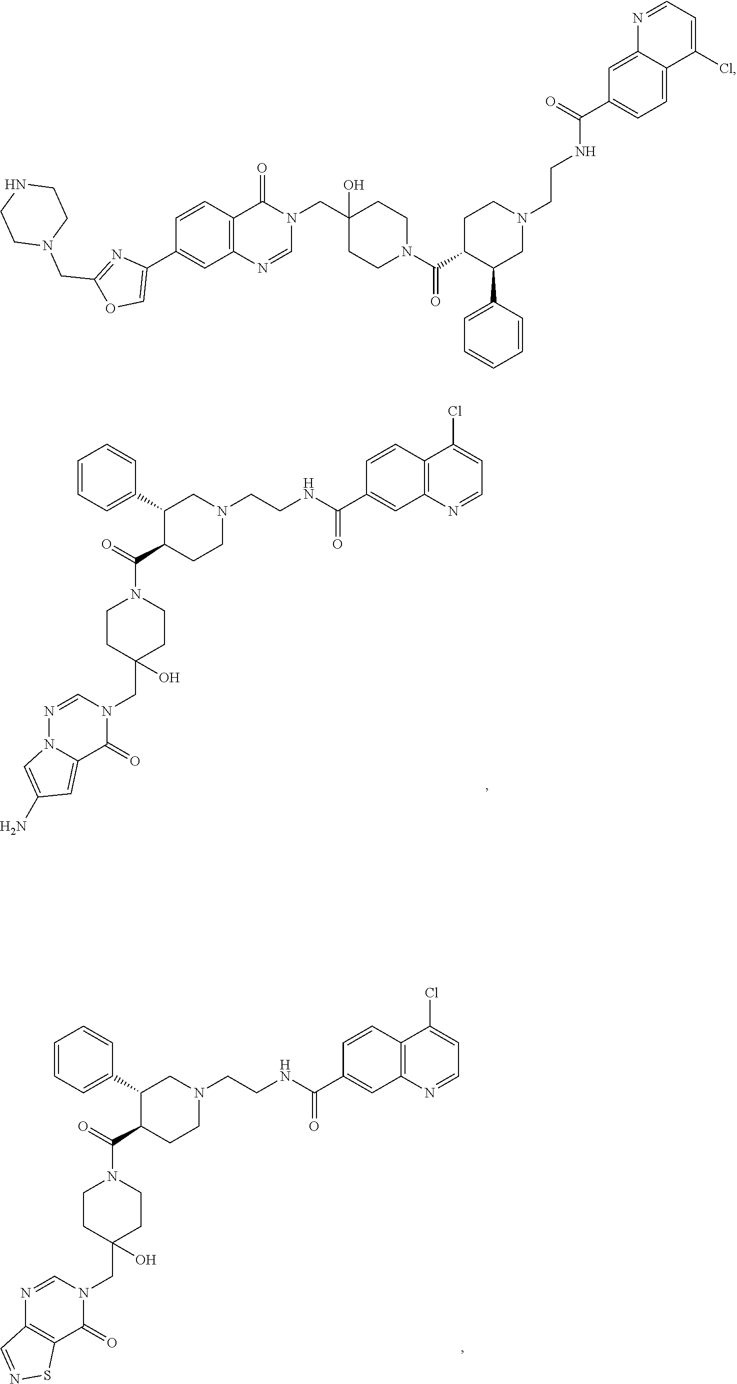 [SMILES: O=C(NCCN1CC[C@@H](C(=O)N2CCC(O)(Cn3cnc4cc(-c5coc(CN6CCNCC6)n5)ccc4c3=O)CC2)[C@H](c2ccccc2)C1)c1ccc2c(Cl)ccnc2c1 ; Nc1cc2c(=O)n(CC3(O)CCN(C(=O)[C@@H]4CCN(CCNC(=O)c5ccc6c(Cl)ccnc6c5)C[C@H]4c4ccccc4)CC3)cnn2c1 ; O=C(NCCN1CC[C@@H](C(=O)N2CCC(O)(Cn3cnc4cnsc4c3=O)CC2)[C@H](c2ccccc2)C1)c1ccc2c(Cl)ccnc2c1] 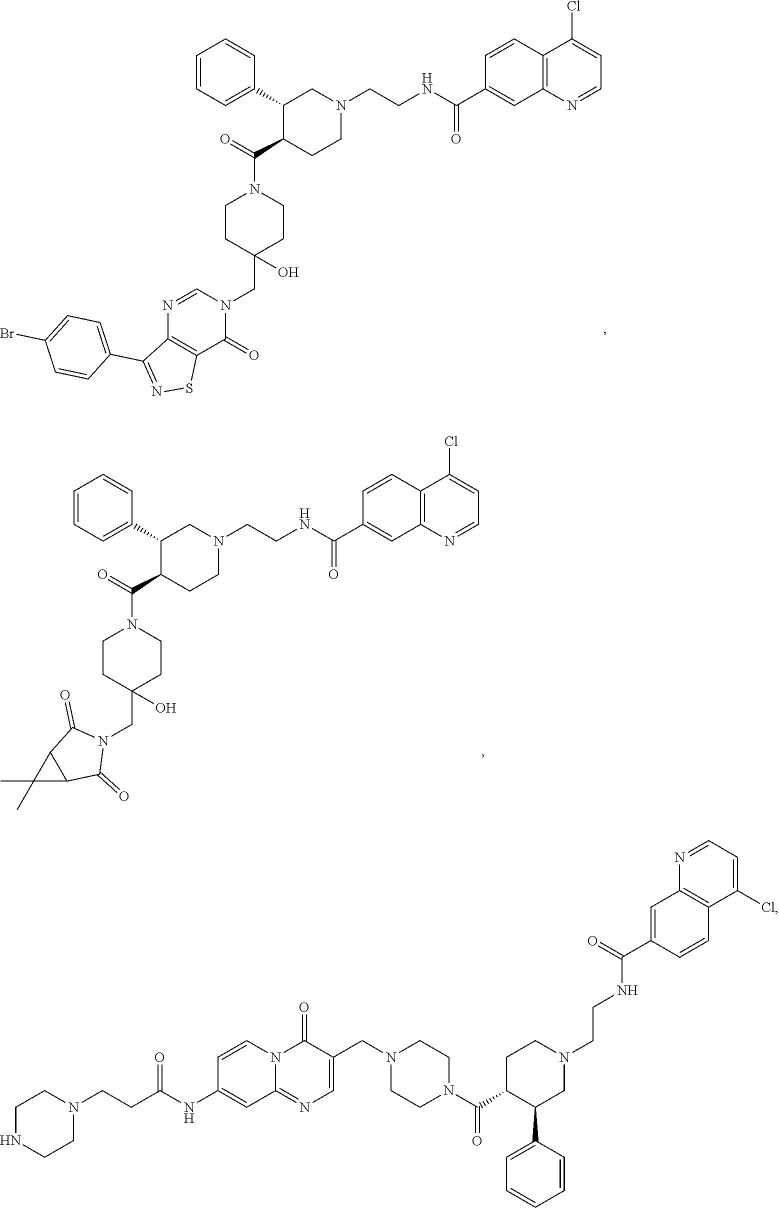 [SMILES: O=C(NCCN1CC[C@@H](C(=O)N2CCC(O)(Cn3cnc4c(-c5ccc(Br)cc5)nsc4c3=O)CC2)[C@H](c2ccccc2)C1)c1ccc2c(Cl)ccnc2c1 ; CC1(C)C2C(=O)N(CC3(O)CCN(C(=O)[C@@H]4CCN(CCNC(=O)c5ccc6c(Cl)ccnc6c5)C[C@H]4c4ccccc4)CC3)C(=O)C21 ; O=C(CCN1CCNCC1)Nc1ccn2c(=O)c(CN3CCN(C(=O)[C@@H]4CCN(CCNC(=O)c5ccc6c(Cl)ccnc6c5)C[C@H]4c4ccccc4)CC3)cnc2c1] 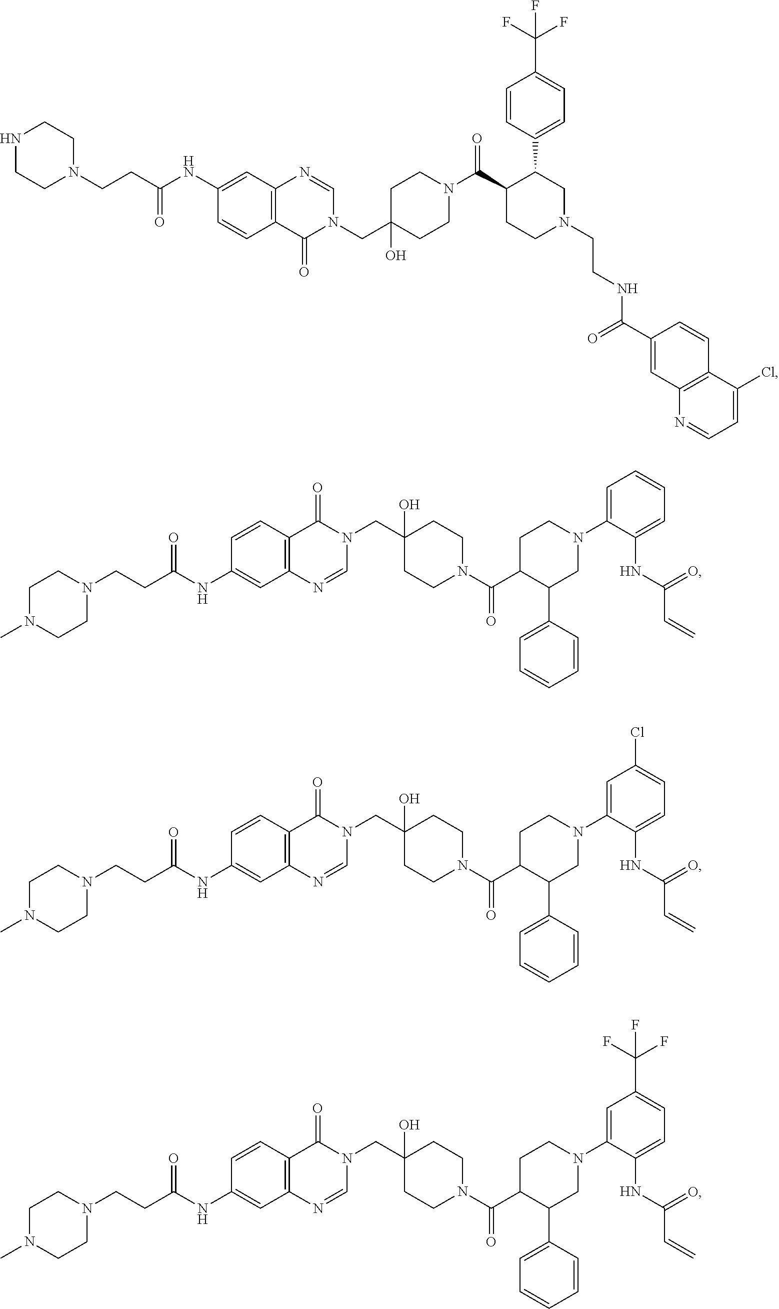 [SMILES: O=C(CCN1CCNCC1)Nc1ccc2c(=O)n(CC3(O)CCN(C(=O)[C@@H]4CCN(CCNC(=O)c5ccc6c(Cl)ccnc6c5)C[C@H]4c4ccc(C(F)(F)F)cc4)CC3)cnc2c1 ; C=CC(=O)Nc1ccccc1N1CCC(C(=O)N2CCC(O)(Cn3cnc4cc(NC(=O)CCN5CCN(C)CC5)ccc4c3=O)CC2)C(c2ccccc2)C1 ; C=CC(=O)Nc1ccc(Cl)cc1N1CCC(C(=O)N2CCC(O)(Cn3cnc4cc(NC(=O)CCN5CCN(C)CC5)ccc4c3=O)CC2)C(c2ccccc2)C1 ; C=CC(=O)Nc1ccc(C(F)(F)F)cc1N1CCC(C(=O)N2CCC(O)(Cn3cnc4cc(NC(=O)CCN5CCN(C)CC5)ccc4c3=O)CC2)C(c2ccccc2)C1] 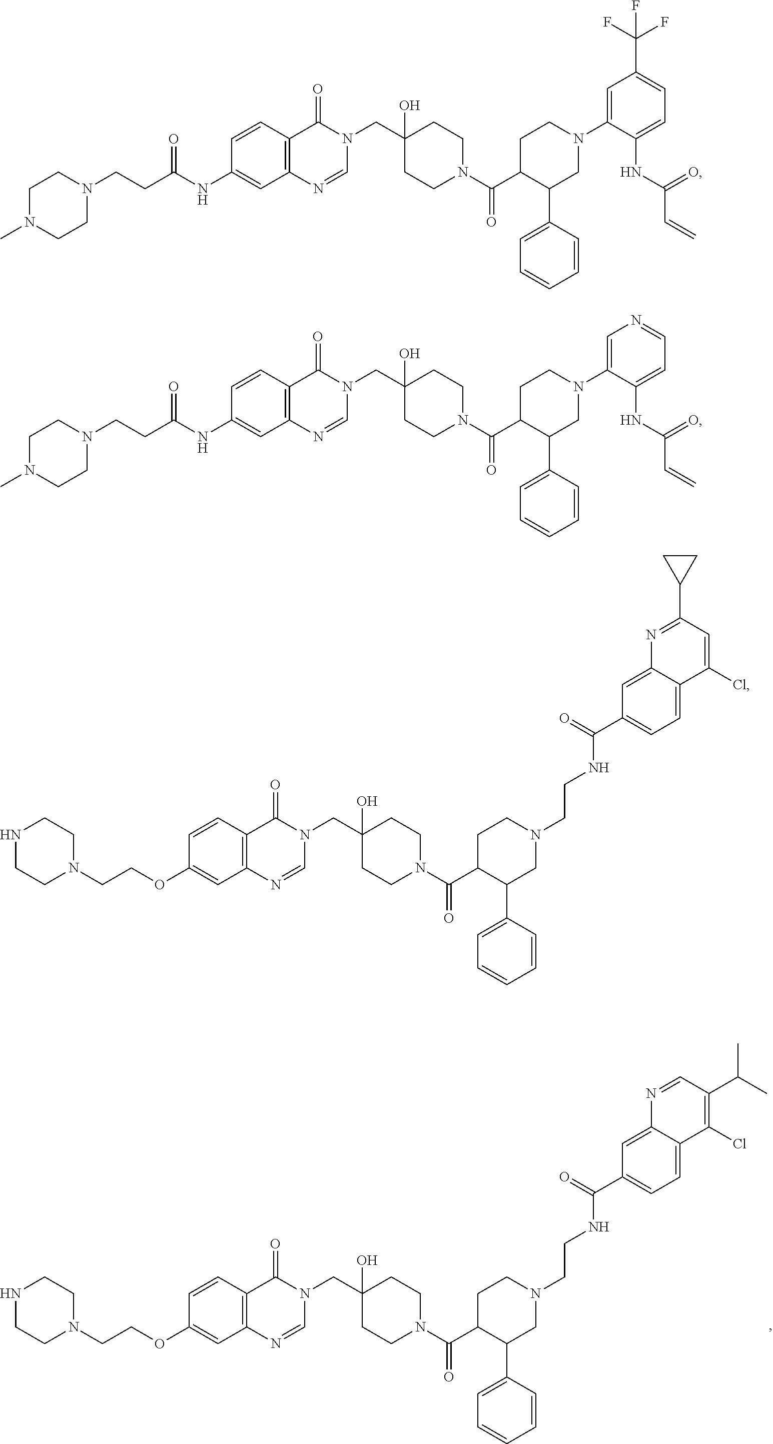 [SMILES: C=CC(=O)Nc1ccc(C(F)(F)F)cc1N1CCC(C(=O)N2CCC(O)(Cn3cnc4cc(NC(=O)CCN5CCN(C)CC5)ccc4c3=O)CC2)C(c2ccccc2)C1 ; C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1 ; O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1 ; CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl] 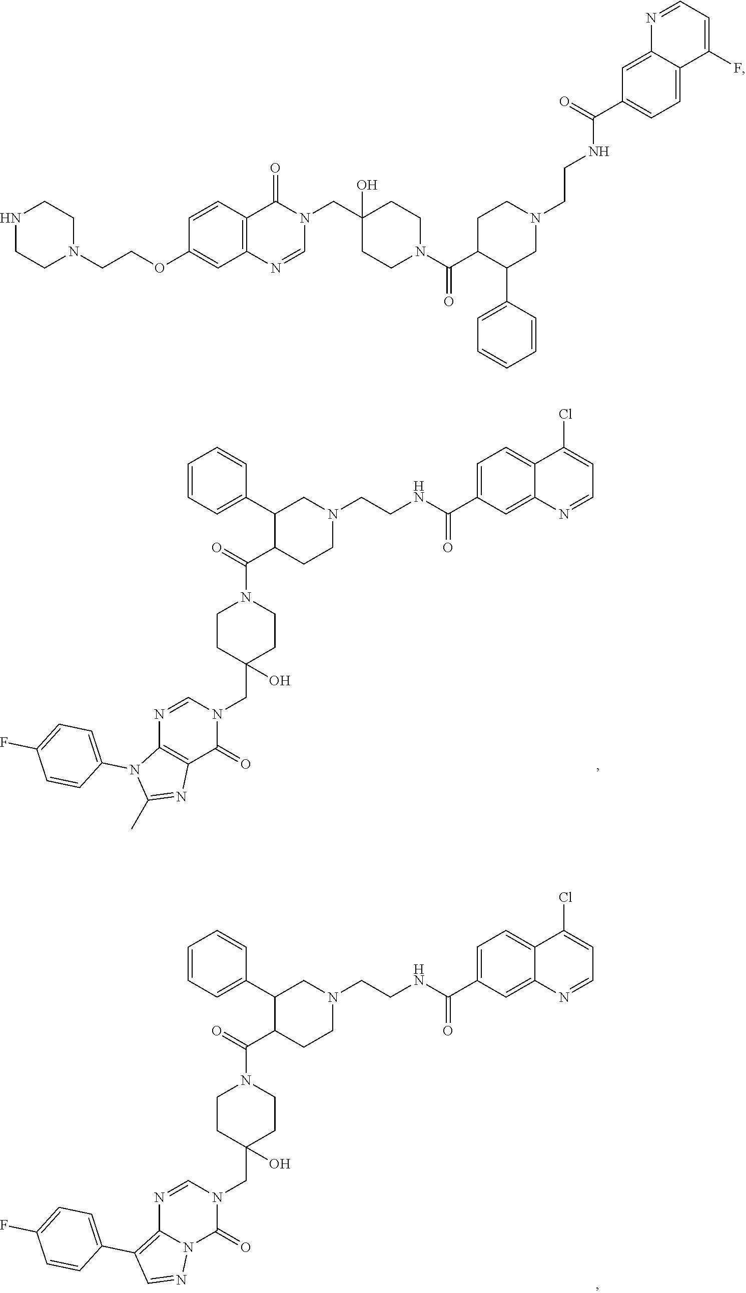 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1 ; Cc1nc2c(=O)n(CC3(O)CCN(C(=O)C4CCN(CCNC(=O)c5ccc6c(Cl)ccnc6c5)CC4c4ccccc4)CC3)cnc2n1-c1ccc(F)cc1 ; O=C(NCCN1CCC(C(=O)N2CCC(O)(Cn3cnc4c(-c5ccc(F)cc5)cnn4c3=O)CC2)C(c2ccccc2)C1)c1ccc2c(Cl)ccnc2c1] 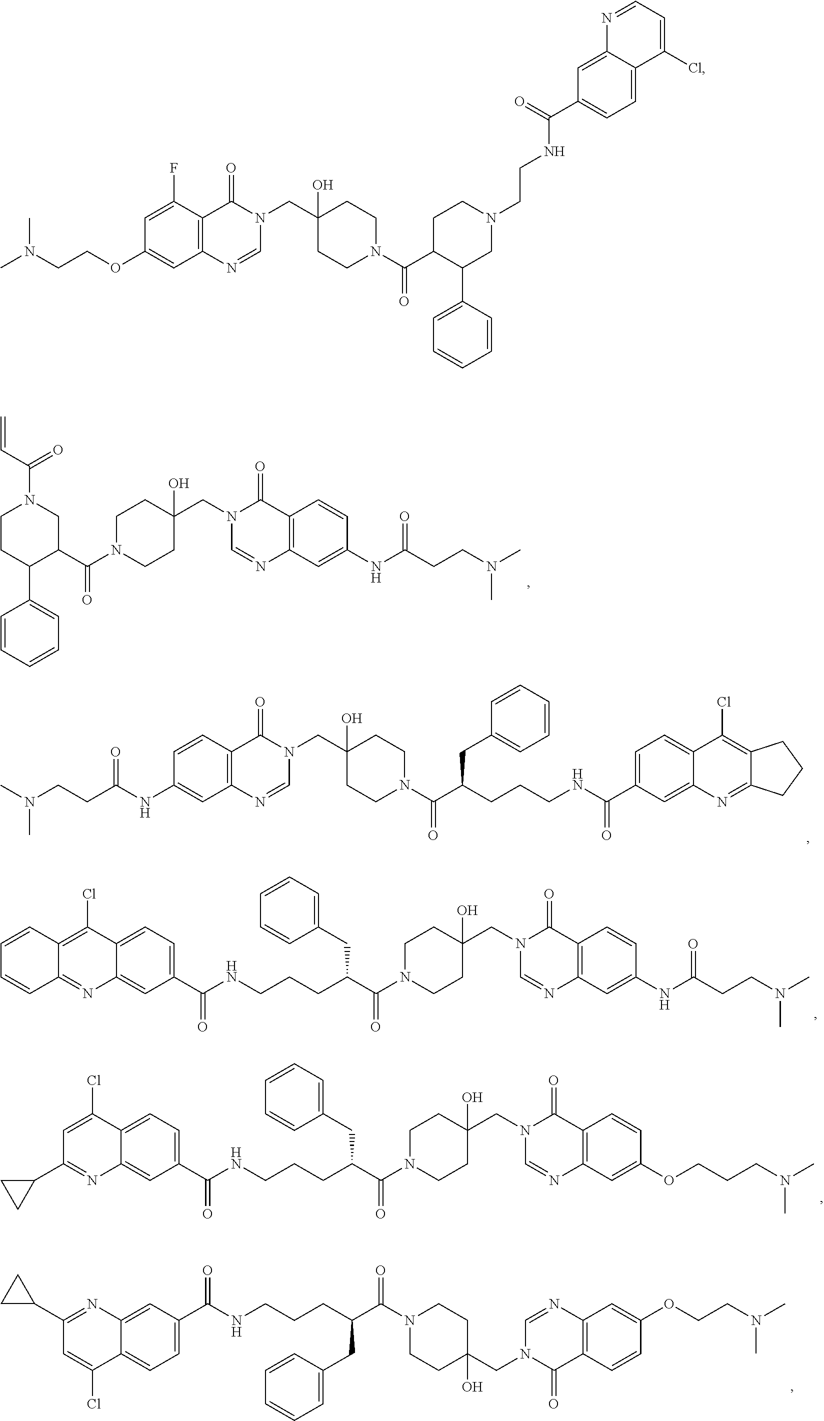 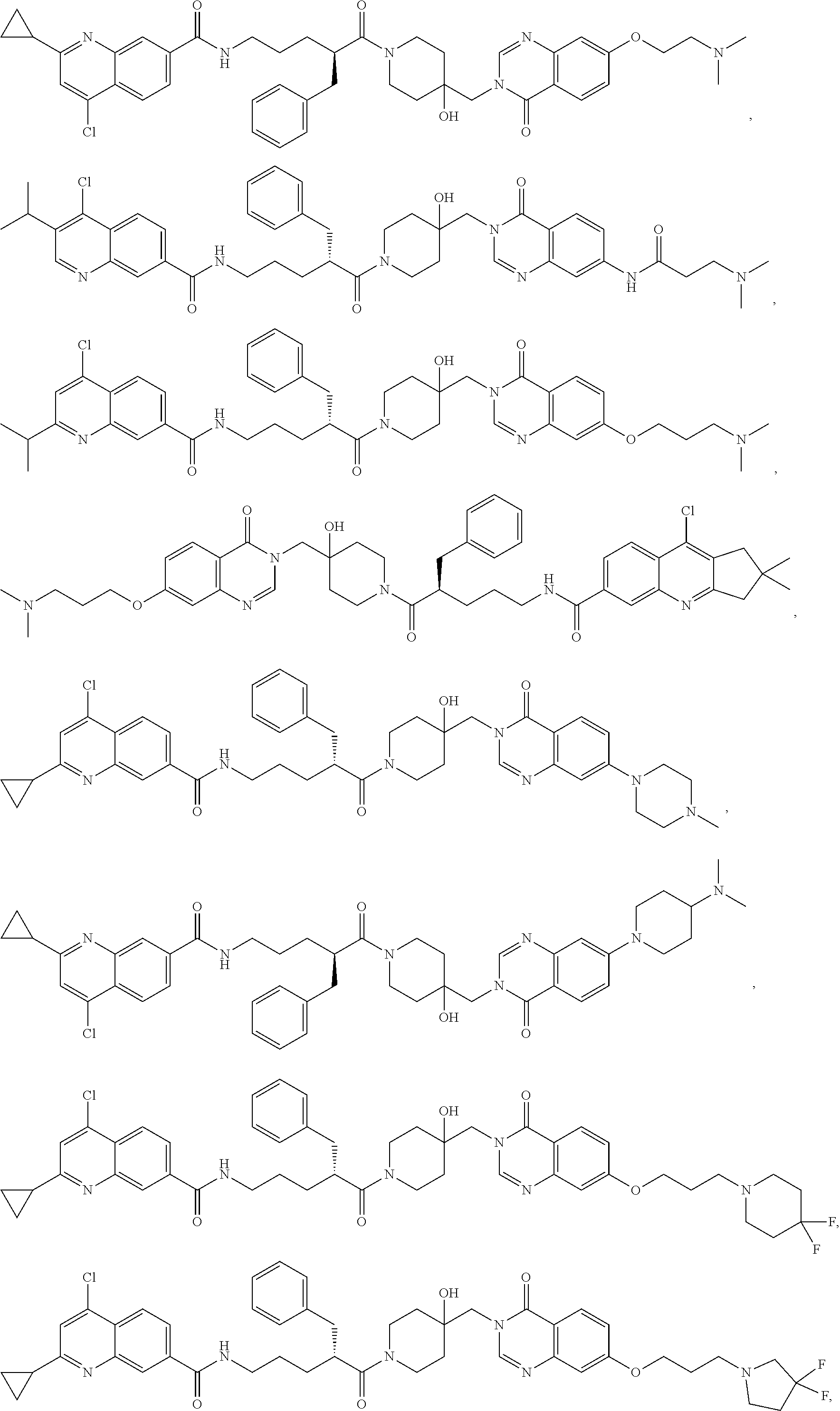 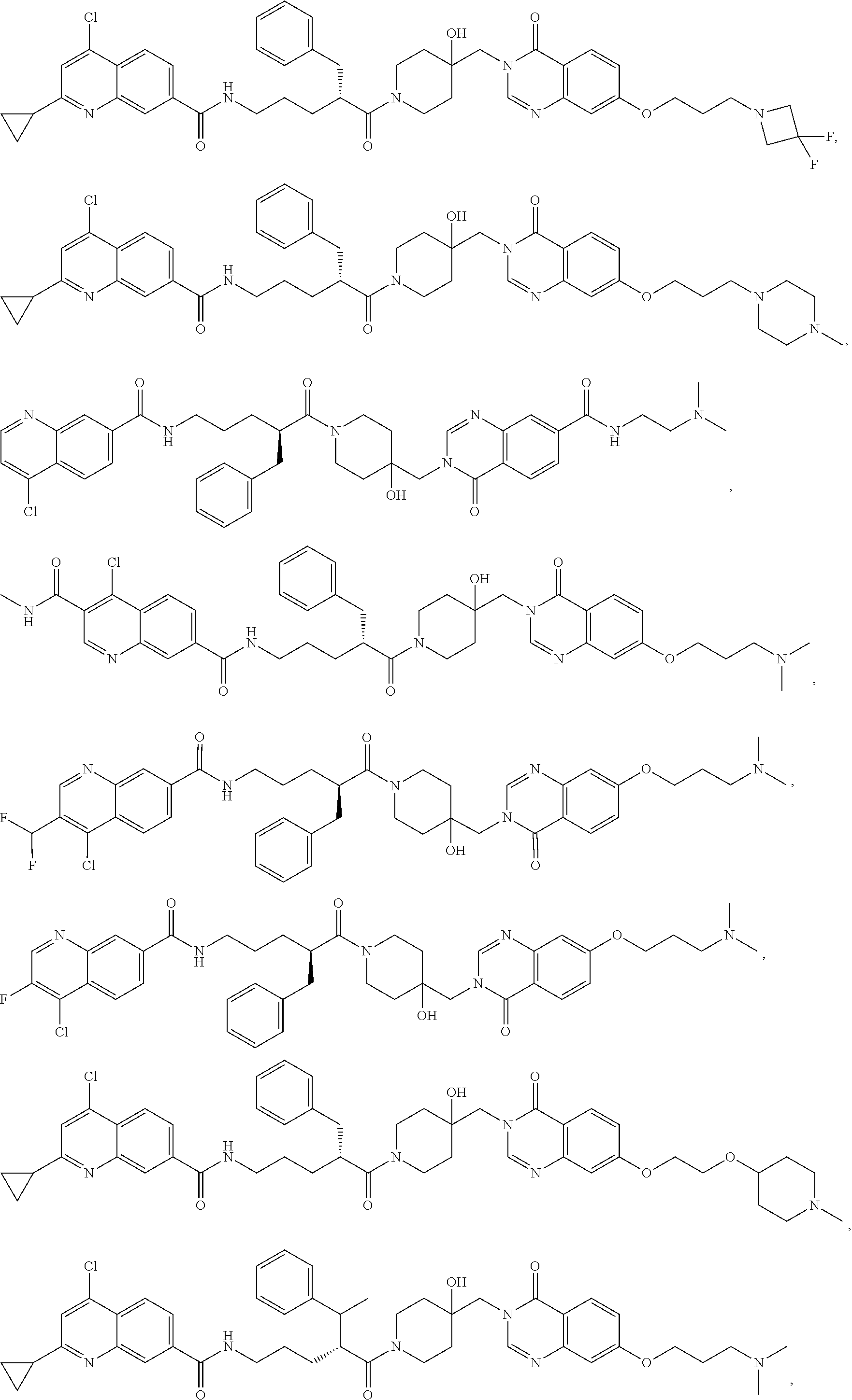 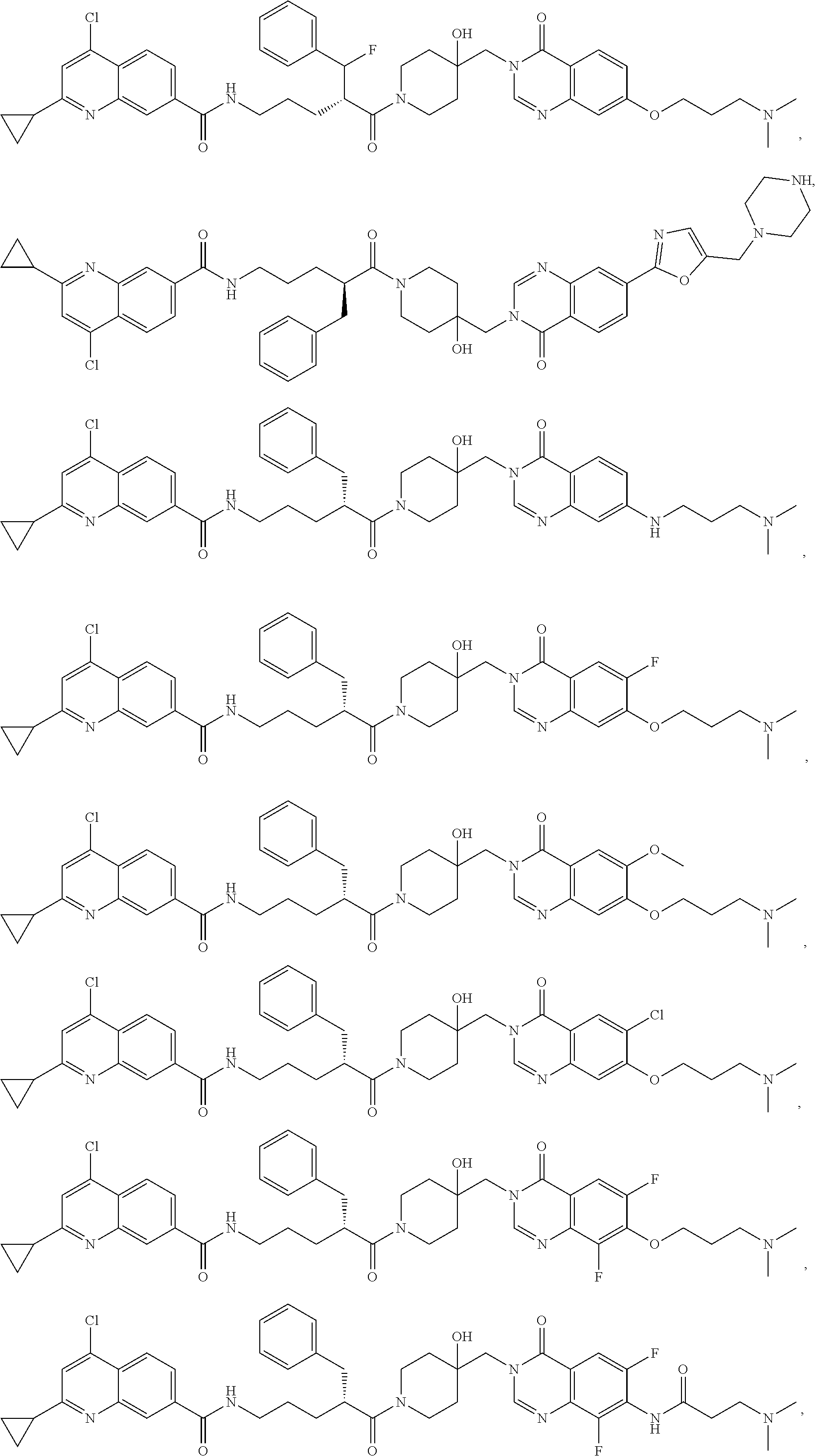 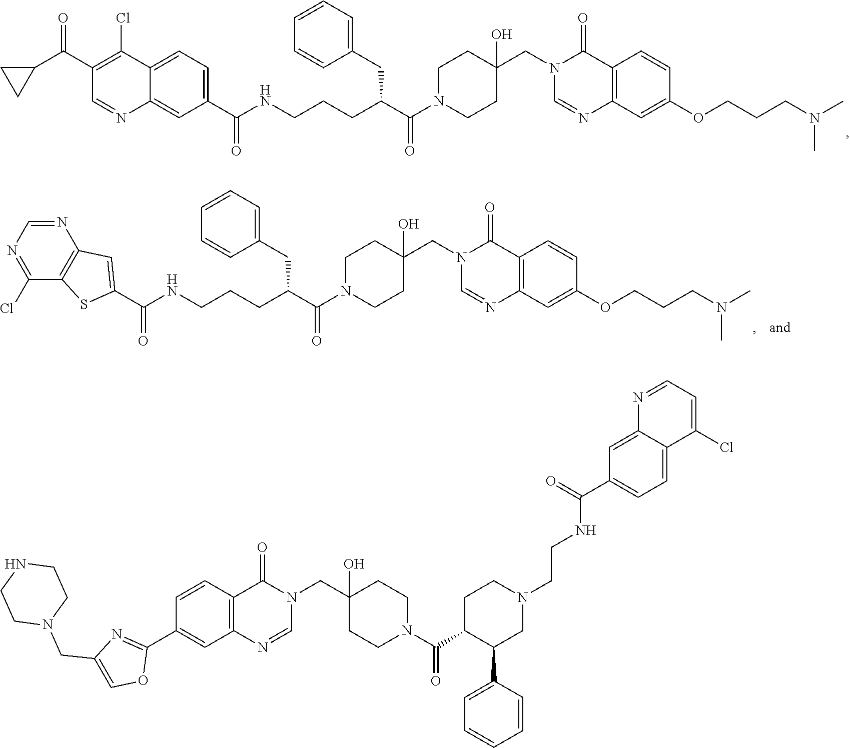
or a pharmaceutically acceptable salt thereof.

74. The compound of claim 73, wherein the compound is selected from 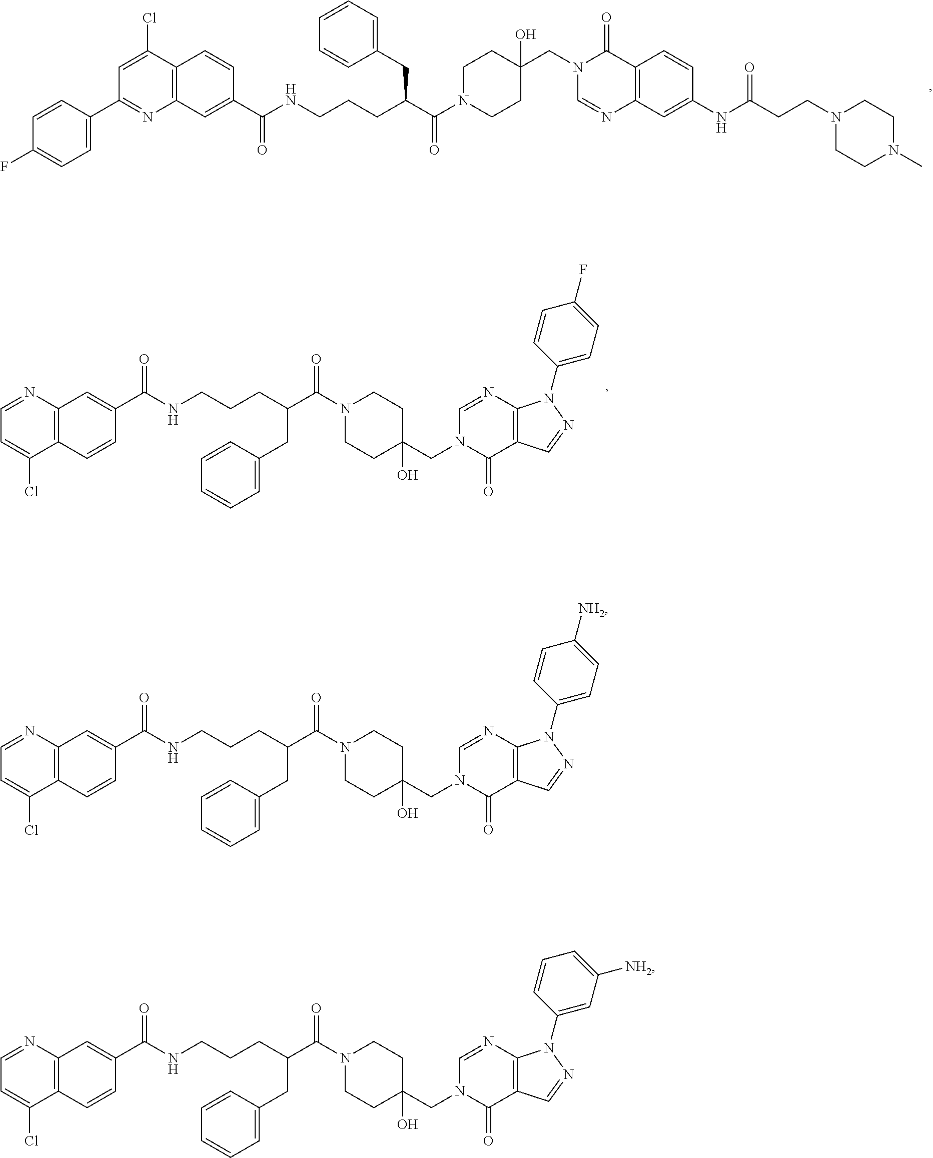 [SMILES: CN1CCN(CCC(=O)Nc2ccc3c(=O)n(CC4(O)CCN(C(=O)[C@@H](CCCNC(=O)c5ccc6c(Cl)cc(-c7ccc(F)cc7)nc6c5)Cc5ccccc5)CC4)cnc3c2)CC1 ; O=C(NCCCC(Cc1ccccc1)C(=O)N1CCC(O)(Cn2cnc3c(cnn3-c3ccc(F)cc3)c2=O)CC1)c1ccc2c(Cl)ccnc2c1 ; Nc1ccc(-n2ncc3c(=O)n(CC4(O)CCN(C(=O)C(CCCNC(=O)c5ccc6c(Cl)ccnc6c5)Cc5ccccc5)CC4)cnc32)cc1 ; Nc1cccc(-n2ncc3c(=O)n(CC4(O)CCN(C(=O)C(CCCNC(=O)c5ccc6c(Cl)ccnc6c5)Cc5ccccc5)CC4)cnc32)c1] 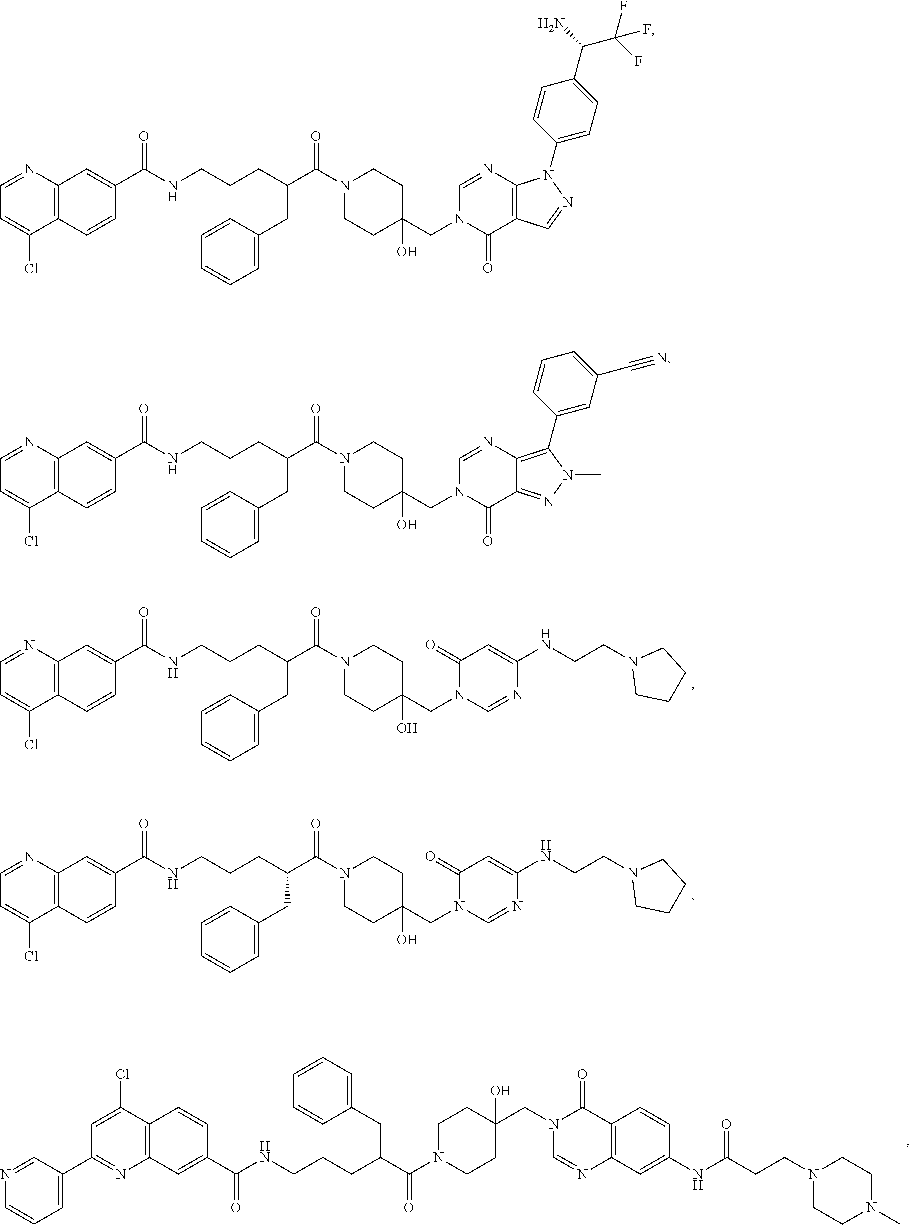 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F ; Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1 ; O=C(NCCCC(Cc1ccccc1)C(=O)N1CCC(O)(Cn2cnc(NCCN3CCCC3)cc2=O)CC1)c1ccc2c(Cl)ccnc2c1 ; O=C(NCCC[C@@H](Cc1ccccc1)C(=O)N1CCC(O)(Cn2cnc(NCCN3CCCC3)cc2=O)CC1)c1ccc2c(Cl)ccnc2c1 ; CN1CCN(CCC(=O)Nc2ccc3c(=O)n(CC4(O)CCN(C(=O)C(CCCNC(=O)c5ccc6c(Cl)cc(-c7cccnc7)nc6c5)Cc5ccccc5)CC4)cnc3c2)CC1] 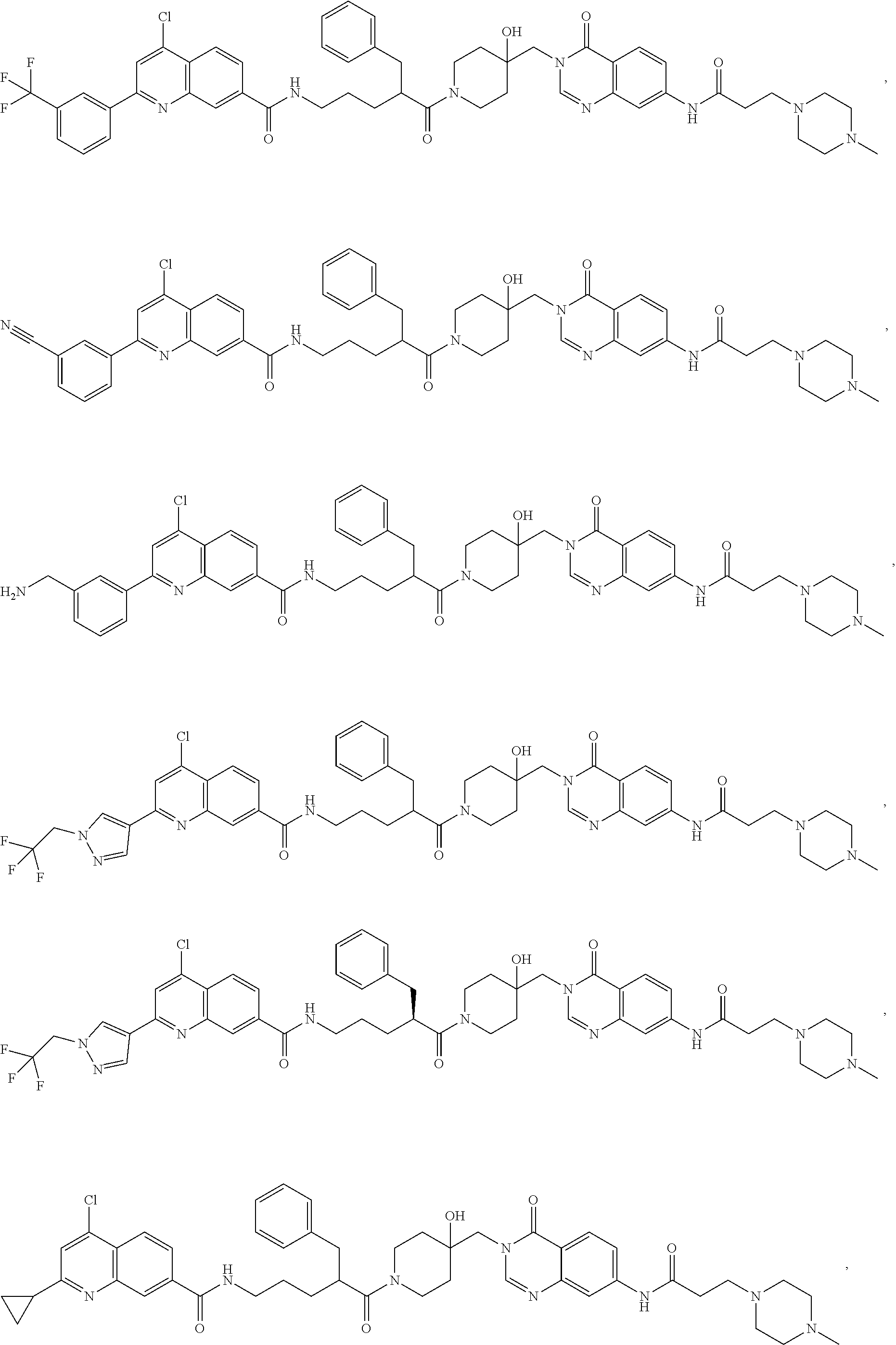 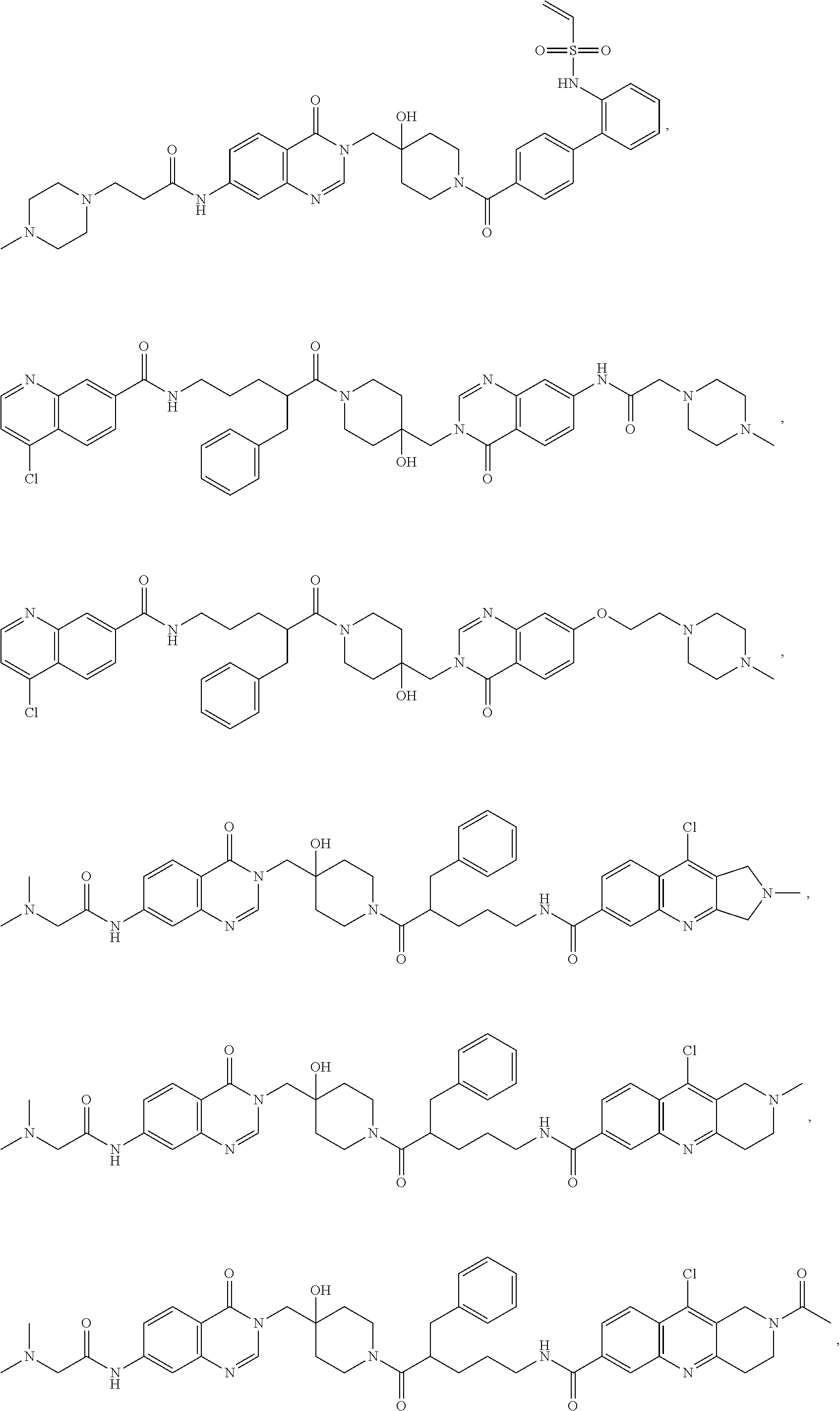 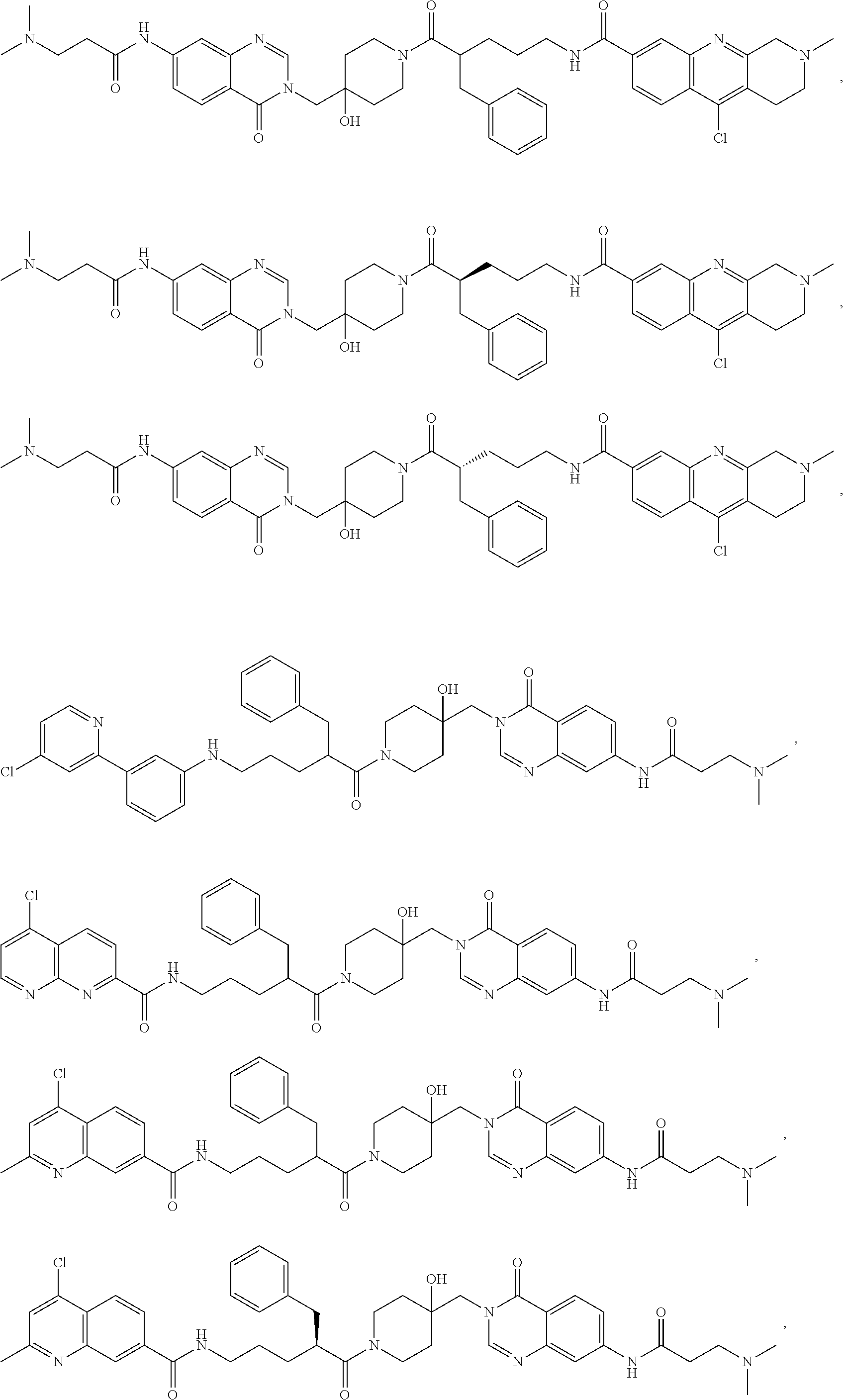 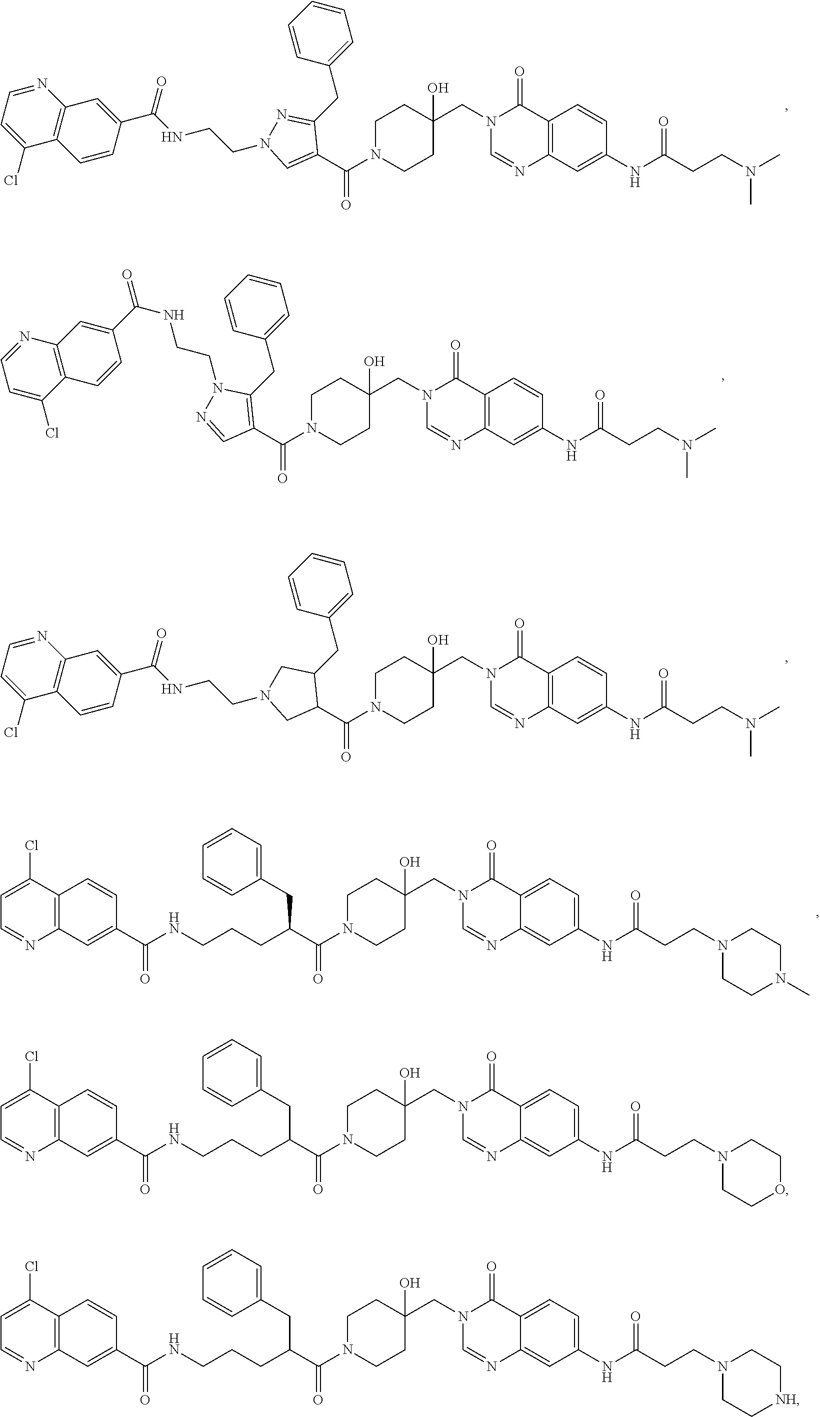 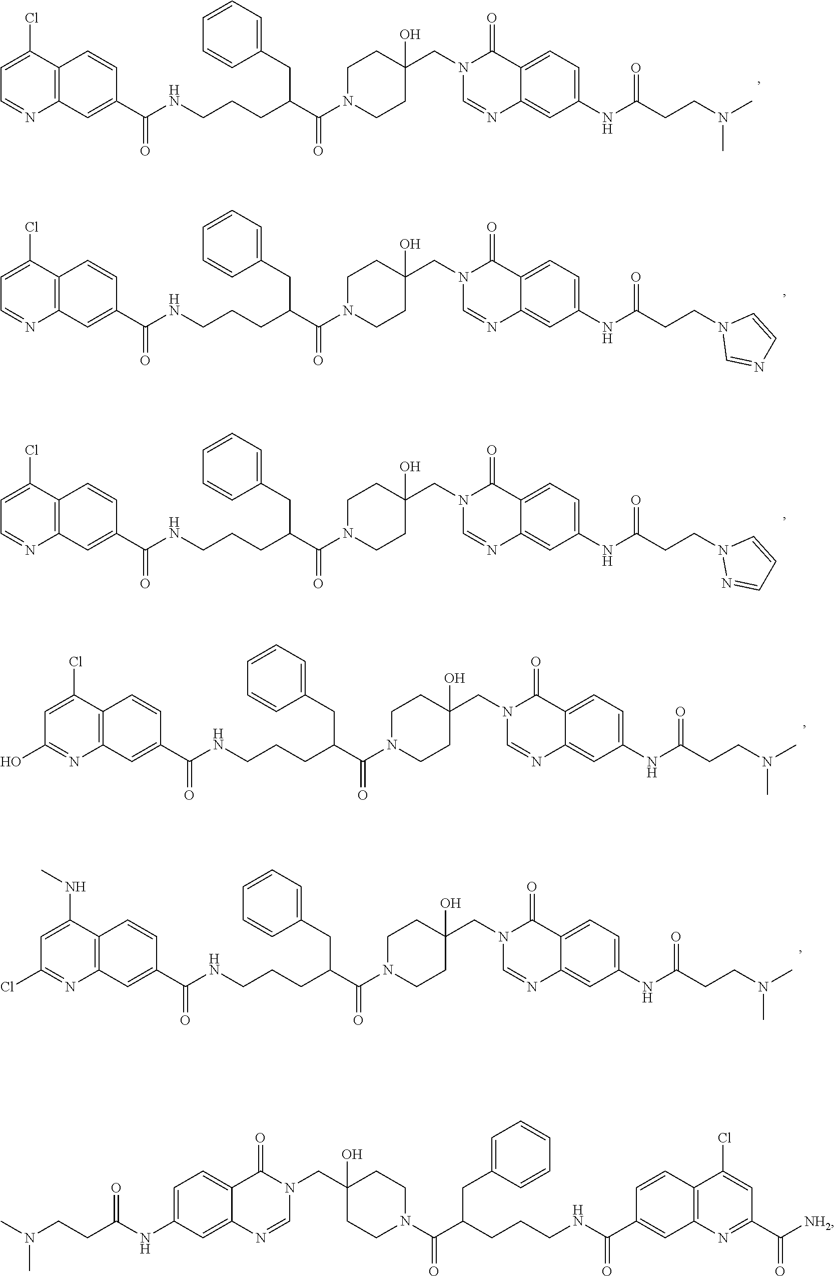 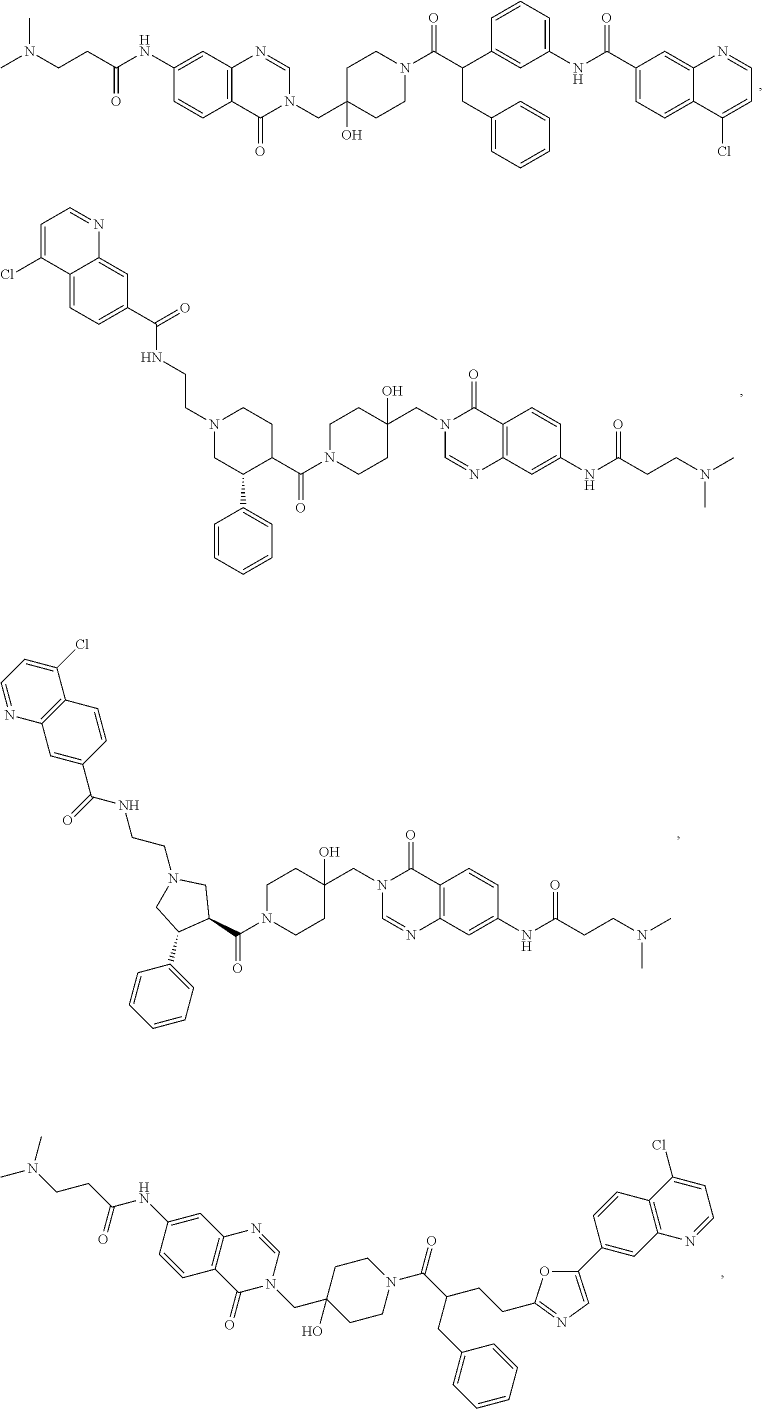 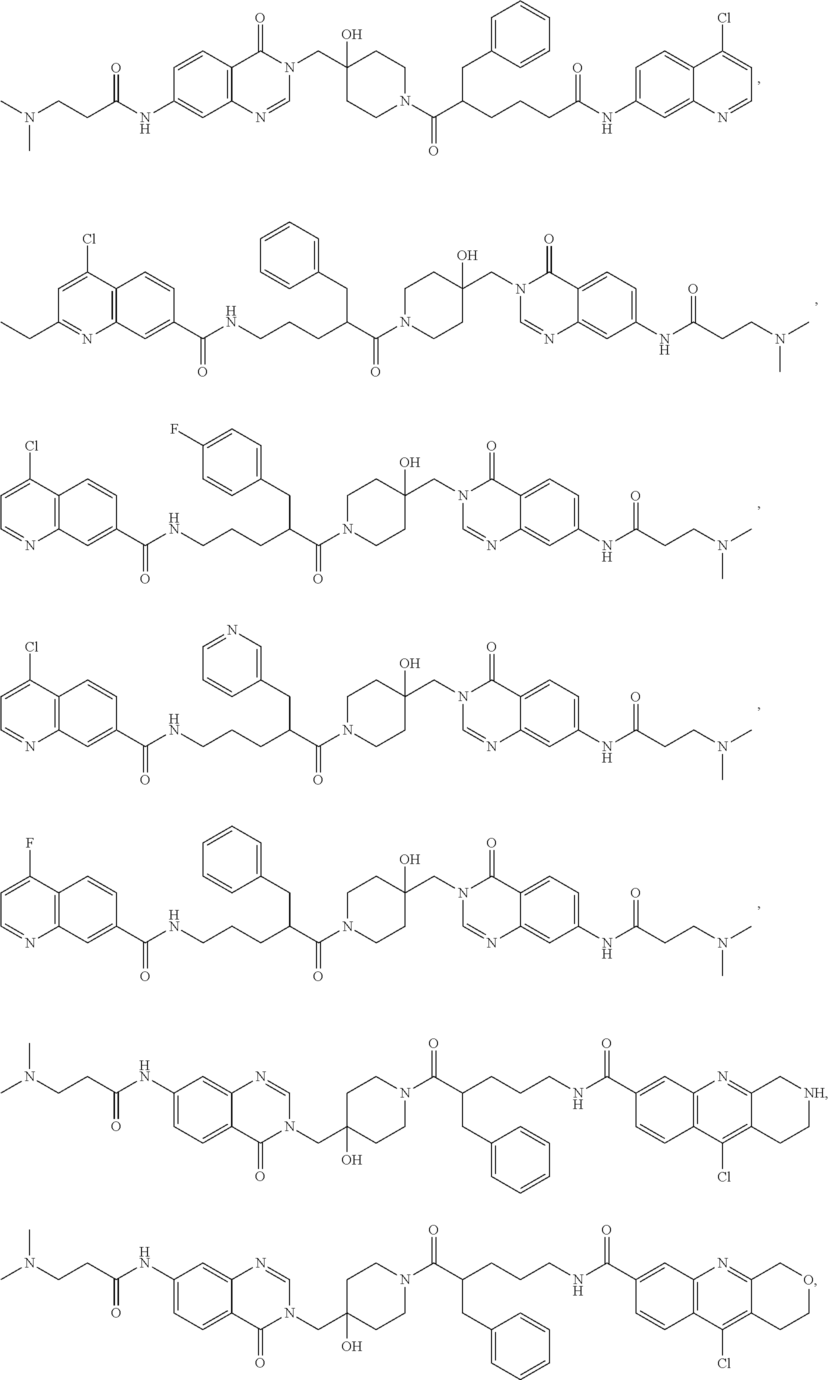 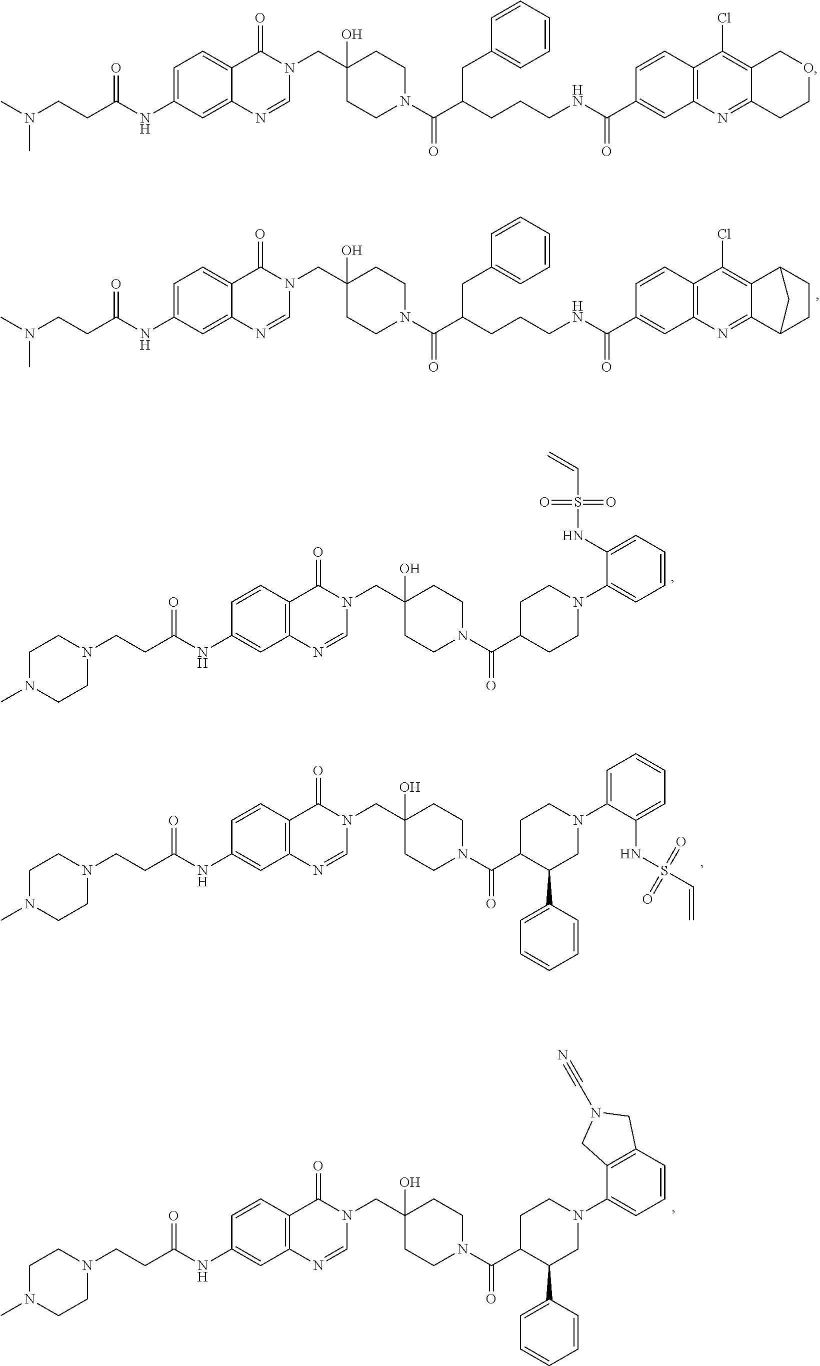 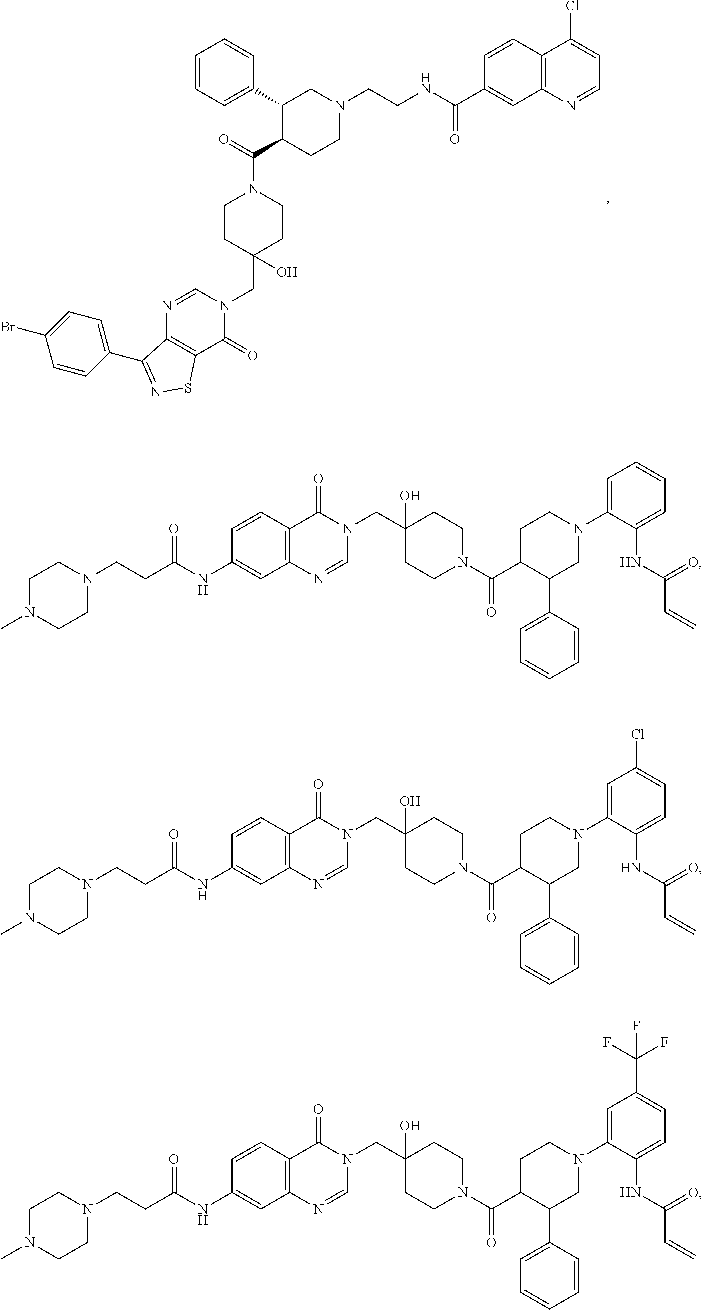 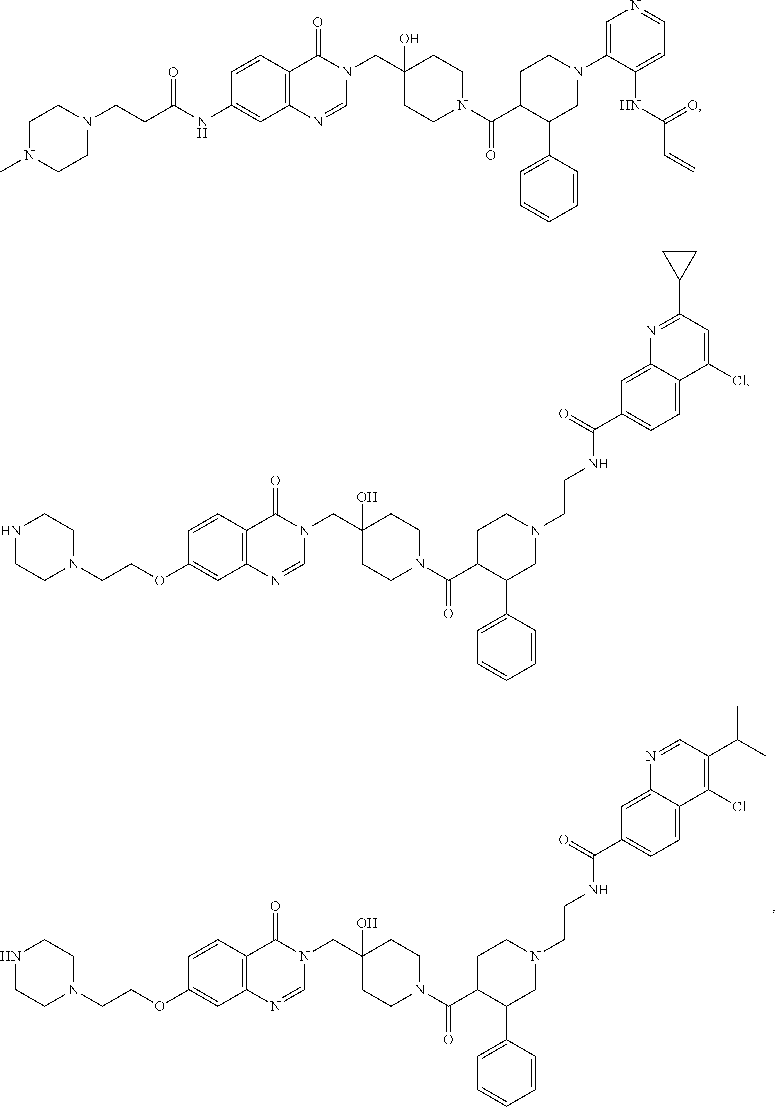 [SMILES: C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1 ; O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1 ; CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl] 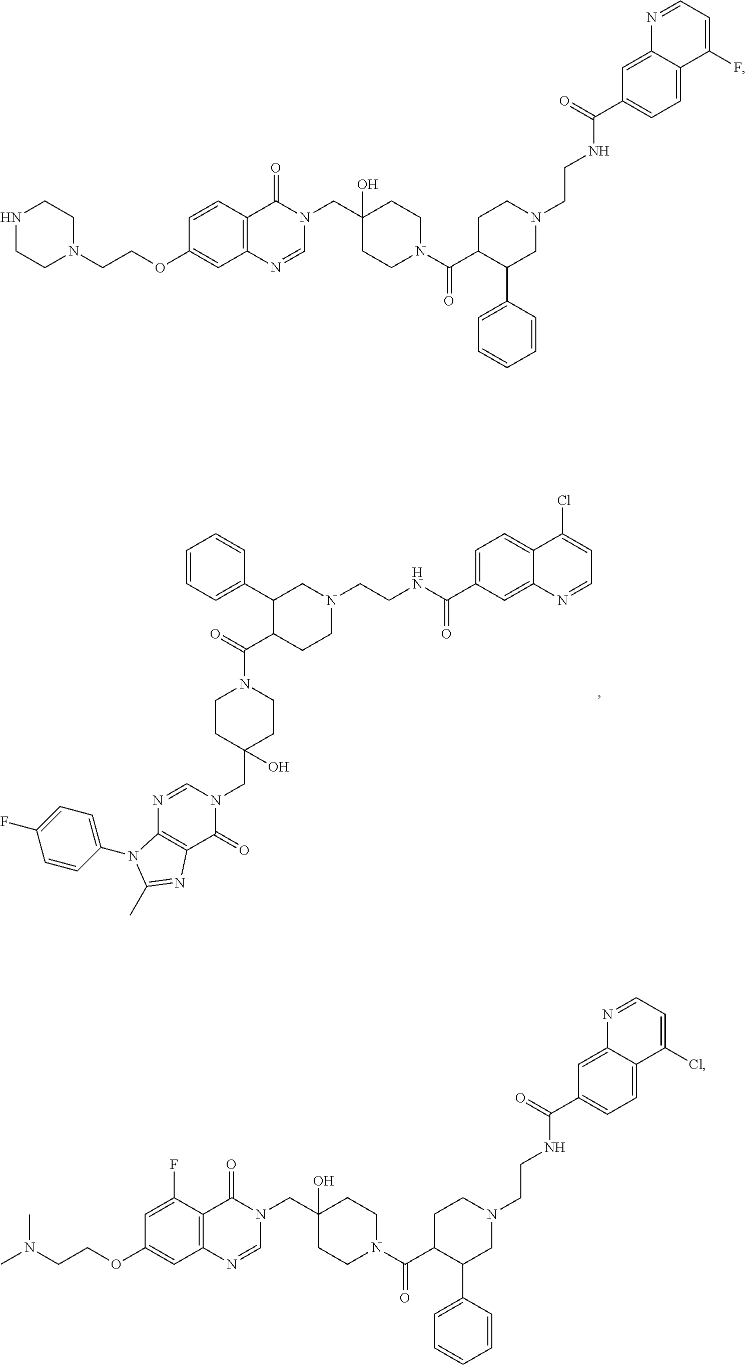 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1 ; Cc1nc2c(=O)n(CC3(O)CCN(C(=O)C4CCN(CCNC(=O)c5ccc6c(Cl)ccnc6c5)CC4c4ccccc4)CC3)cnc2n1-c1ccc(F)cc1 ; CN(C)CCOc1cc(F)c2c(=O)n(CC3(O)CCN(C(=O)C4CCN(CCNC(=O)c5ccc6c(Cl)ccnc6c5)CC4c4ccccc4)CC3)cnc2c1] 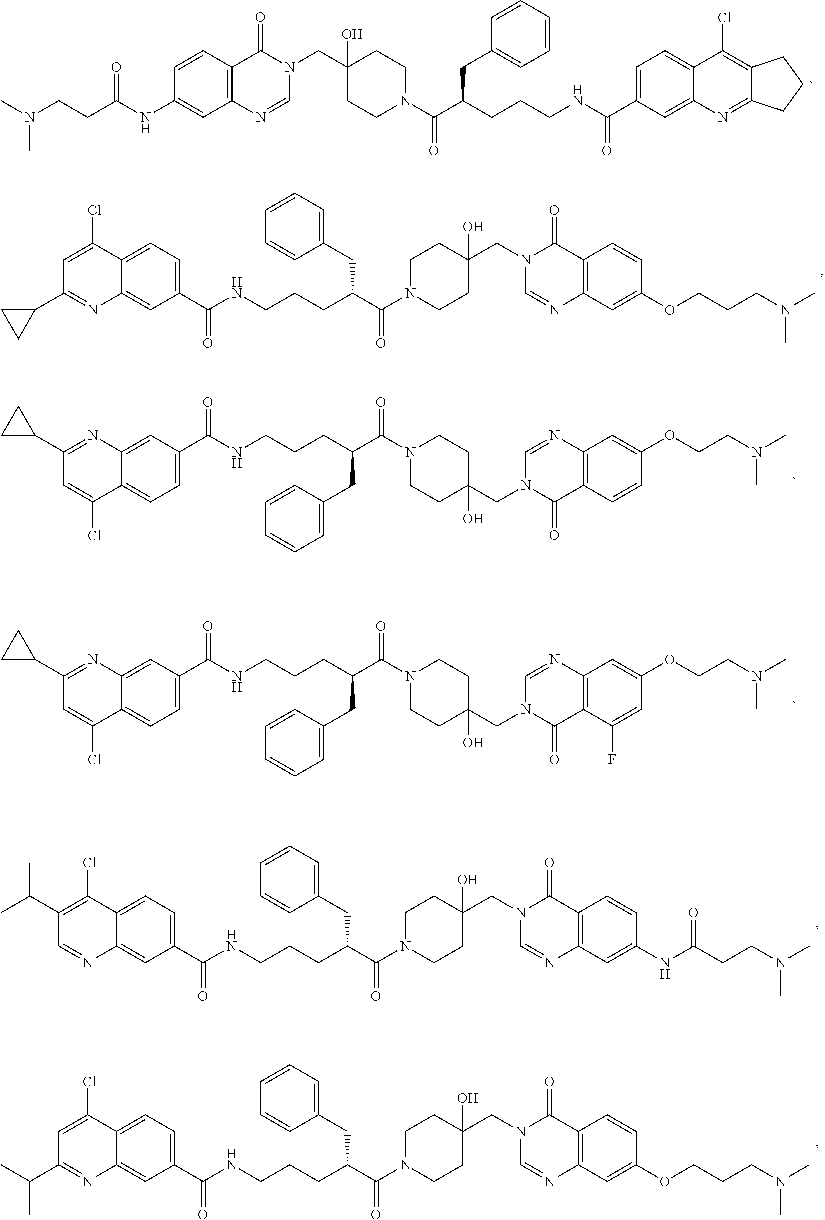 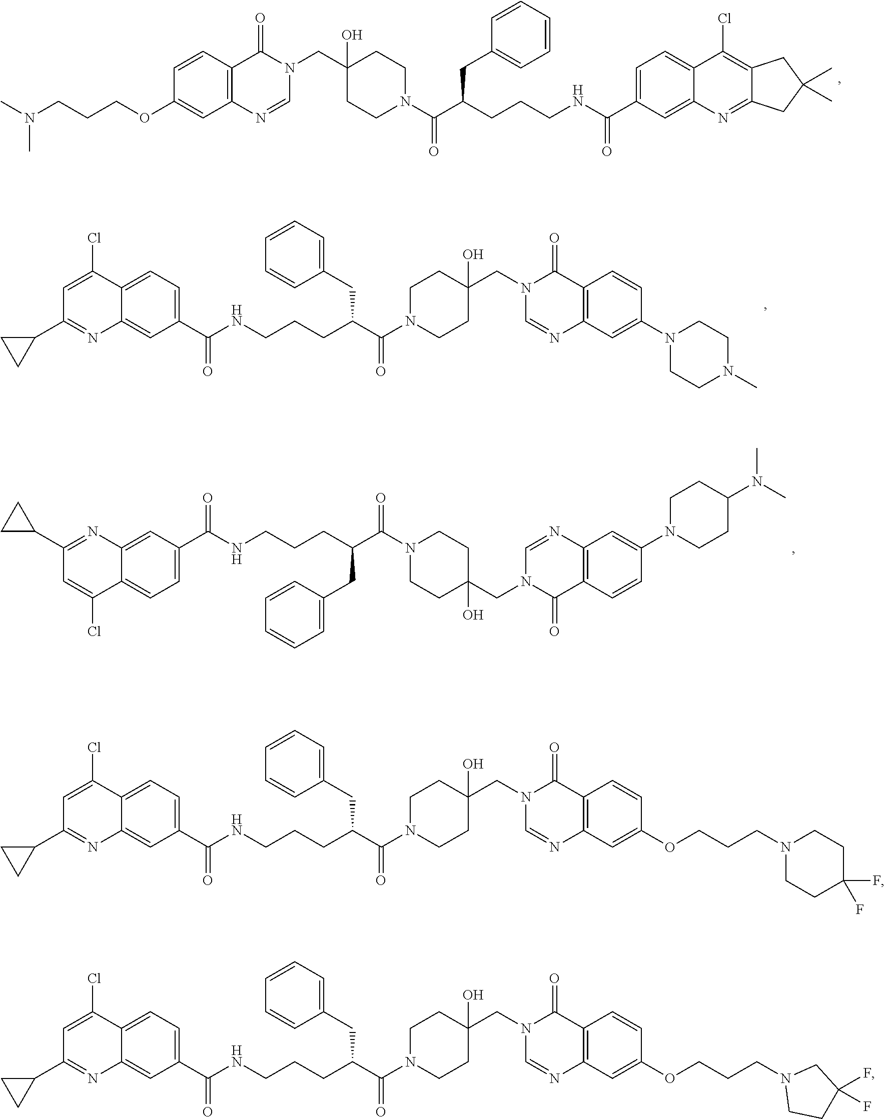 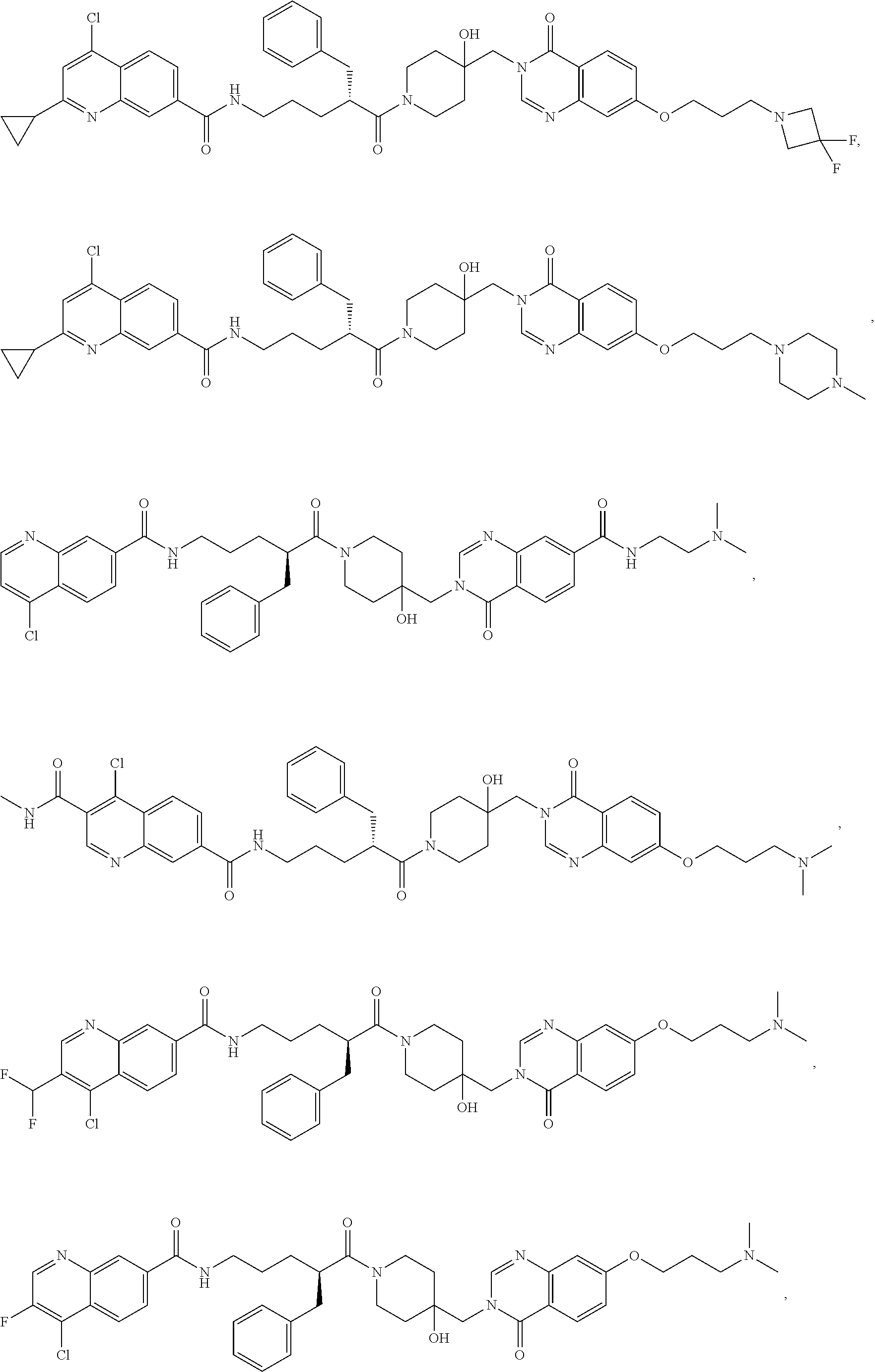 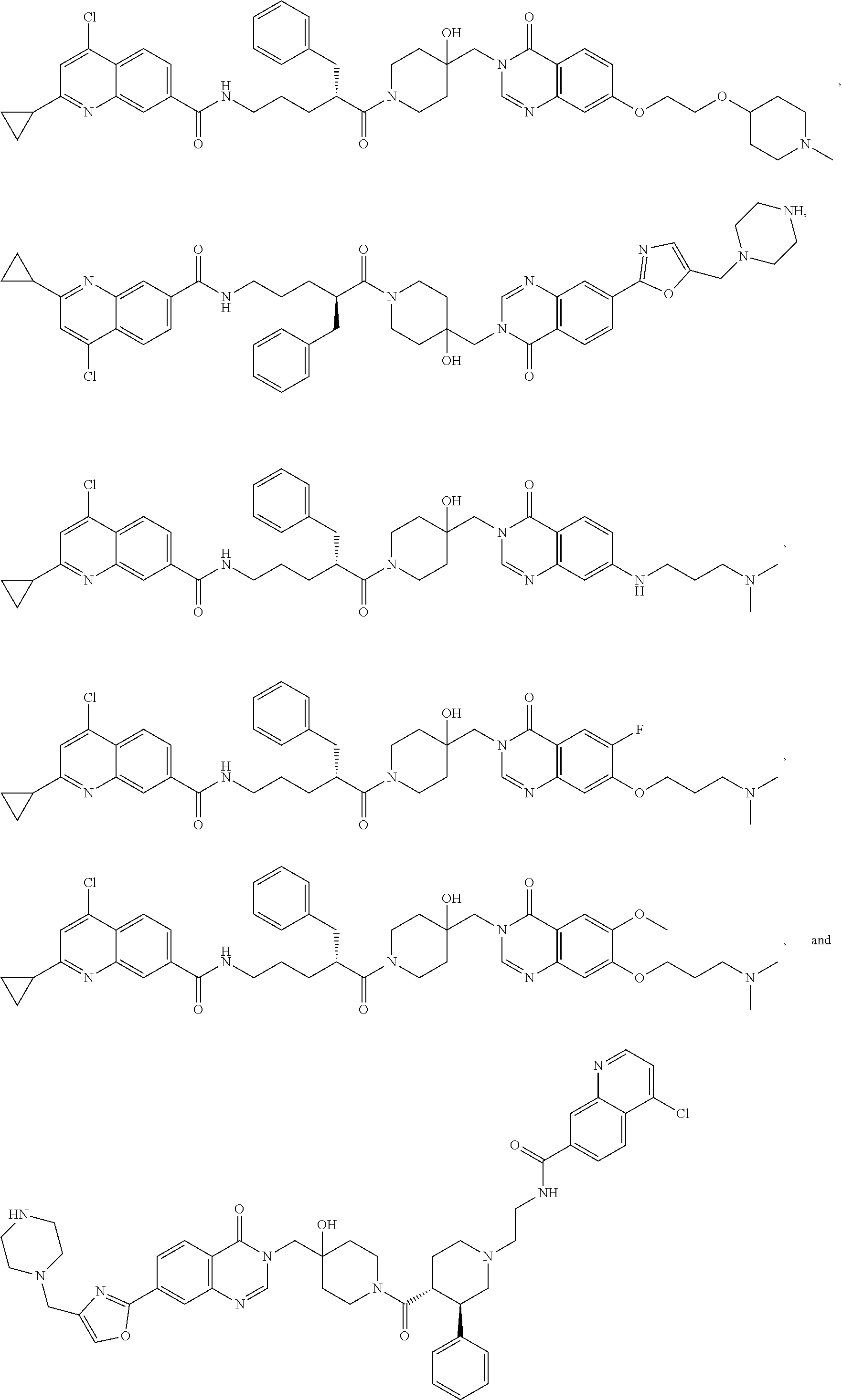
or a pharmaceutically acceptable salt thereof.

75. The compound of claim 73 or 74, wherein the compound is selected from 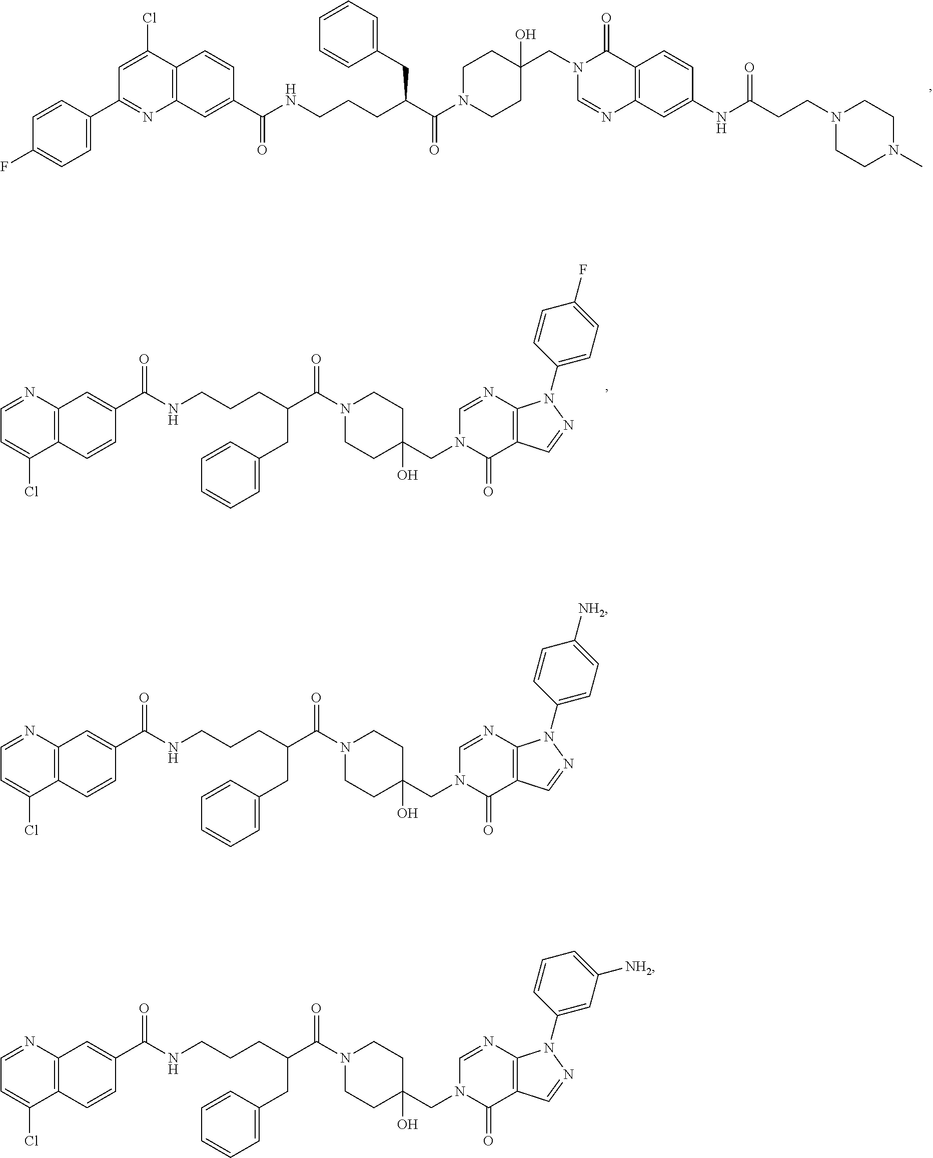 [SMILES: CN1CCN(CCC(=O)Nc2ccc3c(=O)n(CC4(O)CCN(C(=O)[C@@H](CCCNC(=O)c5ccc6c(Cl)cc(-c7ccc(F)cc7)nc6c5)Cc5ccccc5)CC4)cnc3c2)CC1 ; O=C(NCCCC(Cc1ccccc1)C(=O)N1CCC(O)(Cn2cnc3c(cnn3-c3ccc(F)cc3)c2=O)CC1)c1ccc2c(Cl)ccnc2c1 ; Nc1ccc(-n2ncc3c(=O)n(CC4(O)CCN(C(=O)C(CCCNC(=O)c5ccc6c(Cl)ccnc6c5)Cc5ccccc5)CC4)cnc32)cc1 ; Nc1cccc(-n2ncc3c(=O)n(CC4(O)CCN(C(=O)C(CCCNC(=O)c5ccc6c(Cl)ccnc6c5)Cc5ccccc5)CC4)cnc32)c1] 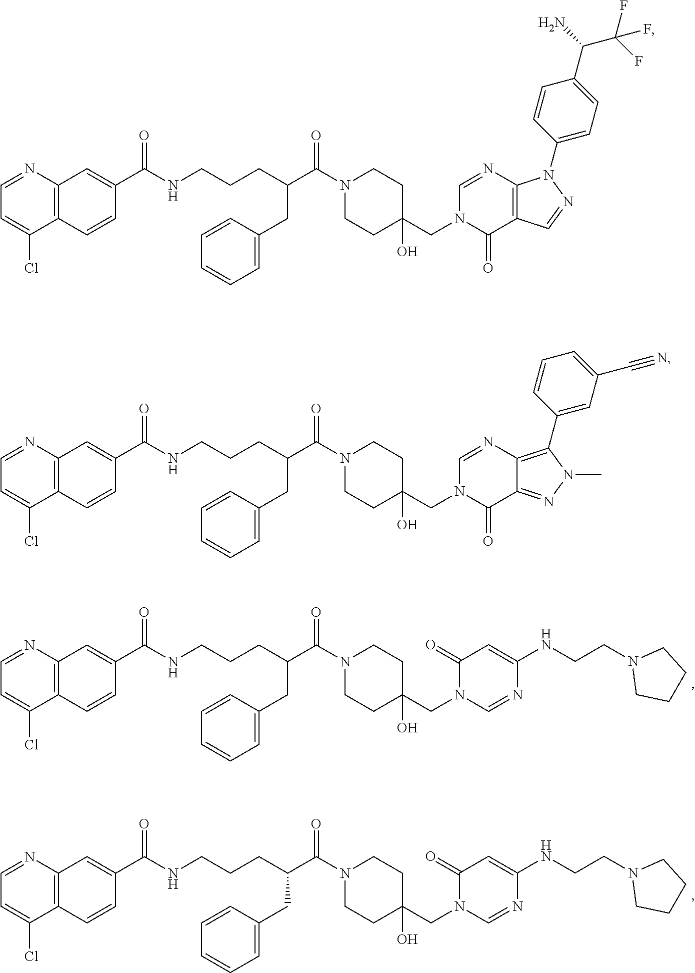 [SMILES: N[C@@H](c1ccc(-n2ncc3c(=O)n(CC4(O)CCN(C(=O)C(CCCNC(=O)c5ccc6c(Cl)ccnc6c5)Cc5ccccc5)CC4)cnc32)cc1)C(F)(F)F ; Cn1nc2c(=O)n(CC3(O)CCN(C(=O)C(CCCNC(=O)c4ccc5c(Cl)ccnc5c4)Cc4ccccc4)CC3)cnc2c1-c1cccc(C#N)c1 ; O=C(NCCCC(Cc1ccccc1)C(=O)N1CCC(O)(Cn2cnc(NCCN3CCCC3)cc2=O)CC1)c1ccc2c(Cl)ccnc2c1 ; O=C(NCCC[C@@H](Cc1ccccc1)C(=O)N1CCC(O)(Cn2cnc(NCCN3CCCC3)cc2=O)CC1)c1ccc2c(Cl)ccnc2c1] 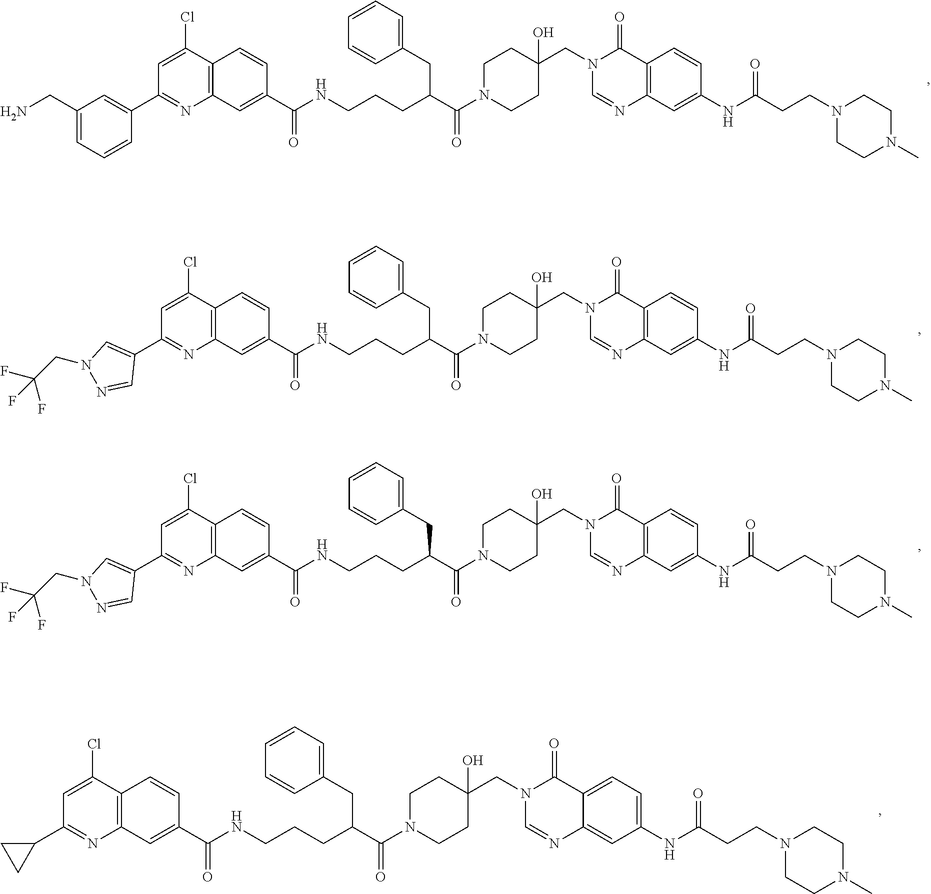 [SMILES: CN1CCN(CCC(=O)Nc2ccc3c(=O)n(CC4(O)CCN(C(=O)C(CCCNC(=O)c5ccc6c(Cl)cc(-c7cccc(CN)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(-c7cnn(CC(F)(F)F)c7)nc6c5)Cc5ccccc5)CC4)cnc3c2)CC1 ; CN1CCN(CCC(=O)Nc2ccc3c(=O)n(CC4(O)CCN(C(=O)[C@@H](CCCNC(=O)c5ccc6c(Cl)cc(-c7cnn(CC(F)(F)F)c7)nc6c5)Cc5ccccc5)CC4)cnc3c2)CC1 ; CN1CCN(CCC(=O)Nc2ccc3c(=O)n(CC4(O)CCN(C(=O)C(CCCNC(=O)c5ccc6c(Cl)cc(C7CC7)nc6c5)Cc5ccccc5)CC4)cnc3c2)CC1] 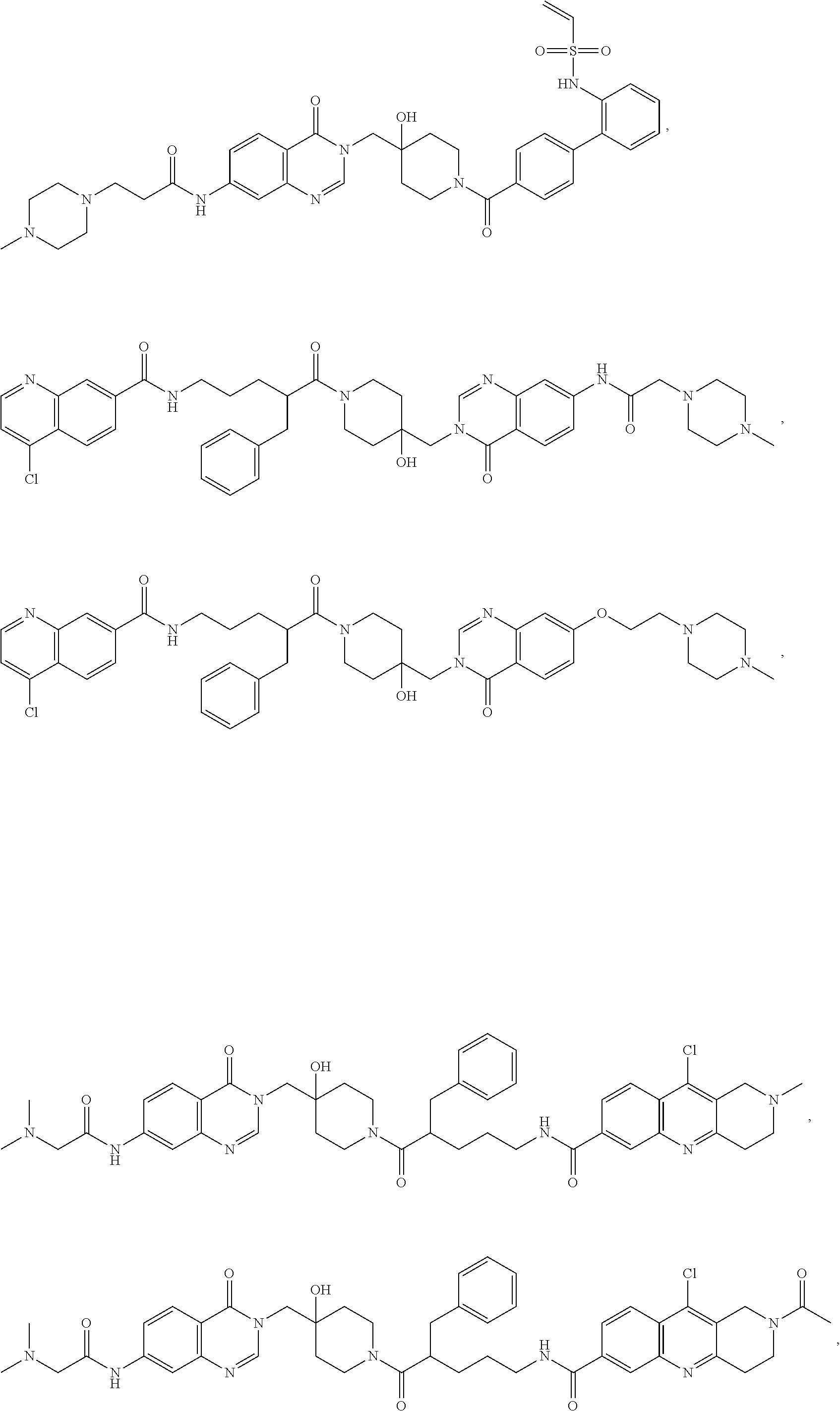 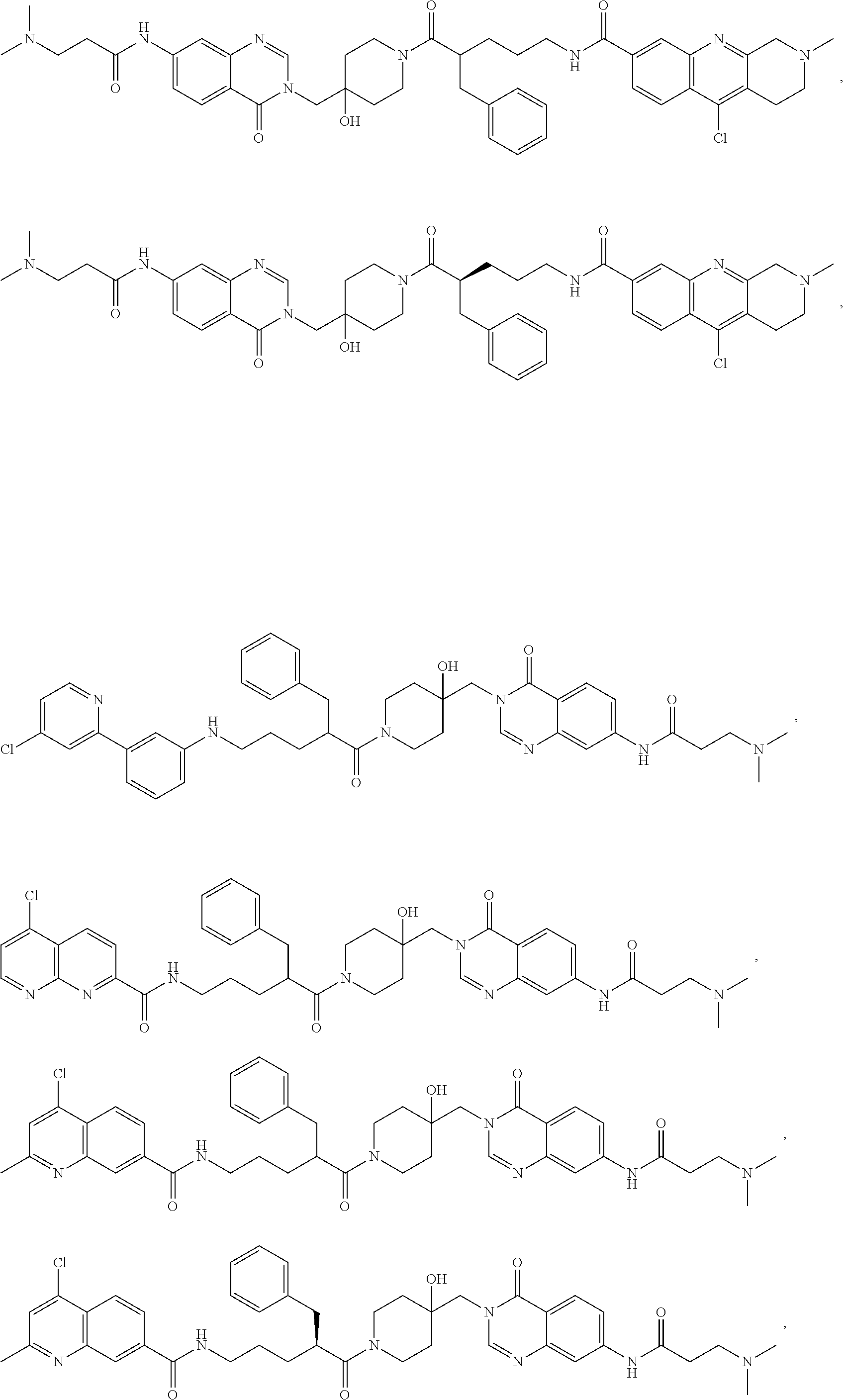 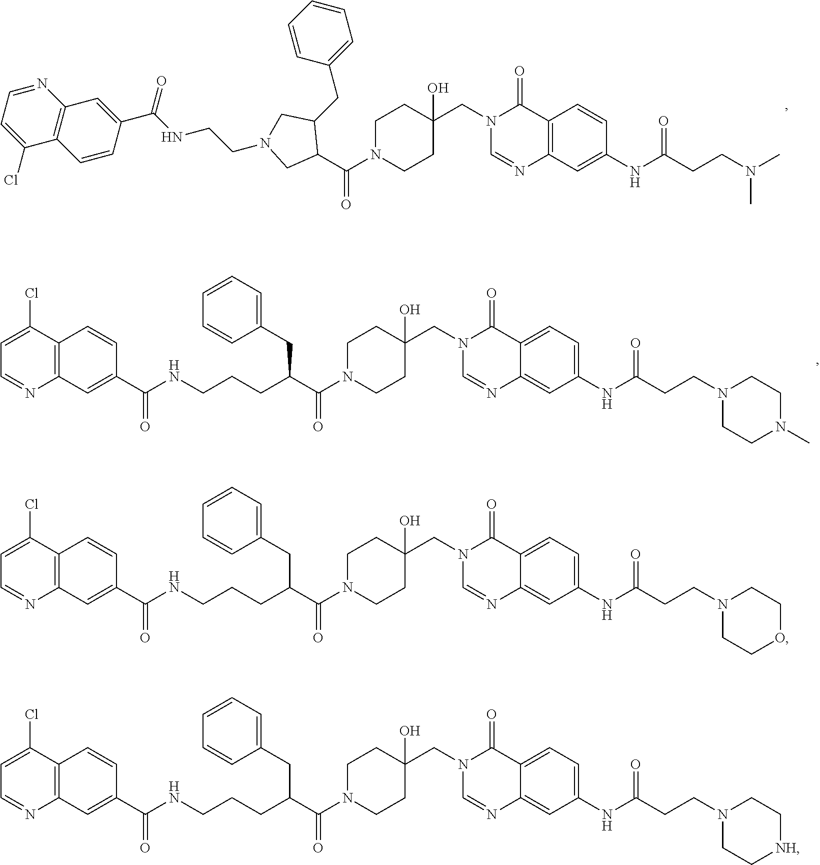 [SMILES: CN(C)CCC(=O)Nc1ccc2c(=O)n(CC3(O)CCN(C(=O)C4CN(CCNC(=O)c5ccc6c(Cl)ccnc6c5)CC4Cc4ccccc4)CC3)cnc2c1 ; CN1CCN(CCC(=O)Nc2ccc3c(=O)n(CC4(O)CCN(C(=O)[C@@H](CCCNC(=O)c5ccc6c(Cl)ccnc6c5)Cc5ccccc5)CC4)cnc3c2)CC1 ; O=C(CCN1CCOCC1)Nc1ccc2c(=O)n(CC3(O)CCN(C(=O)C(CCCNC(=O)c4ccc5c(Cl)ccnc5c4)Cc4ccccc4)CC3)cnc2c1 ; O=C(CCN1CCNCC1)Nc1ccc2c(=O)n(CC3(O)CCN(C(=O)C(CCCNC(=O)c4ccc5c(Cl)ccnc5c4)Cc4ccccc4)CC3)cnc2c1] 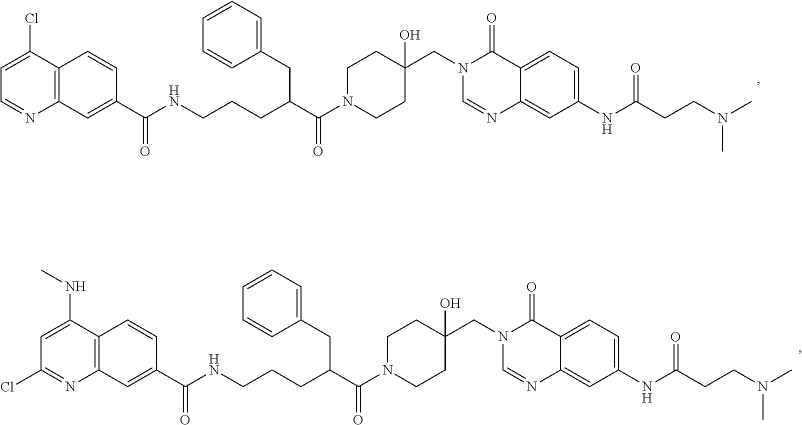 [SMILES: CN(C)CCC(=O)Nc1ccc2c(=O)n(CC3(O)CCN(C(=O)C(CCCNC(=O)c4ccc5c(Cl)ccnc5c4)Cc4ccccc4)CC3)cnc2c1 ; CNc1cc(Cl)nc2cc(C(=O)NCCCC(Cc3ccccc3)C(=O)N3CCC(O)(Cn4cnc5cc(NC(=O)CCN(C)C)ccc5c4=O)CC3)ccc12] 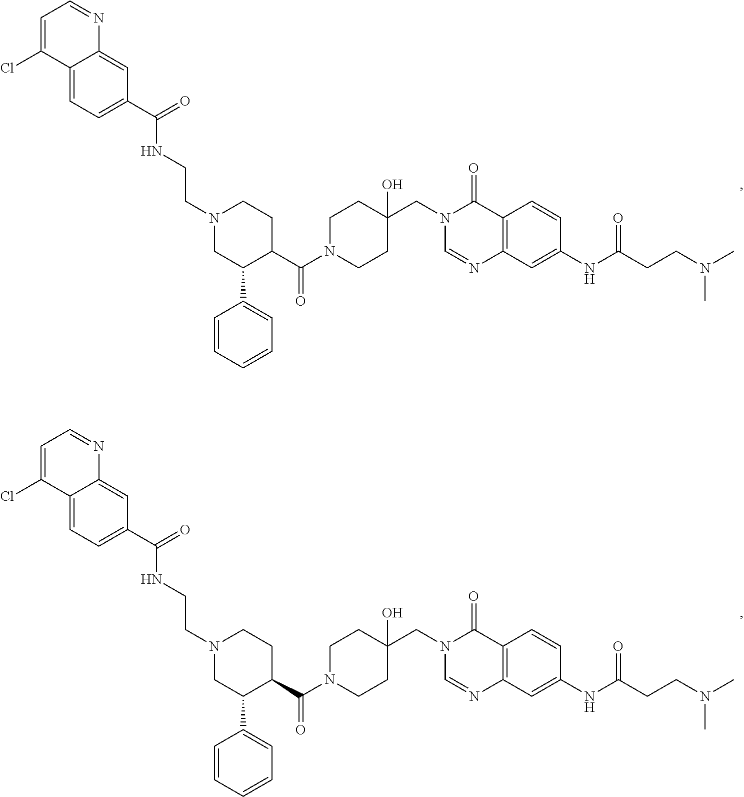 [SMILES: CN(C)CCC(=O)Nc1ccc2c(=O)n(CC3(O)CCN(C(=O)C4CCN(CCNC(=O)c5ccc6c(Cl)ccnc6c5)C[C@H]4c4ccccc4)CC3)cnc2c1 ; CN(C)CCC(=O)Nc1ccc2c(=O)n(CC3(O)CCN(C(=O)[C@@H]4CCN(CCNC(=O)c5ccc6c(Cl)ccnc6c5)C[C@H]4c4ccccc4)CC3)cnc2c1] 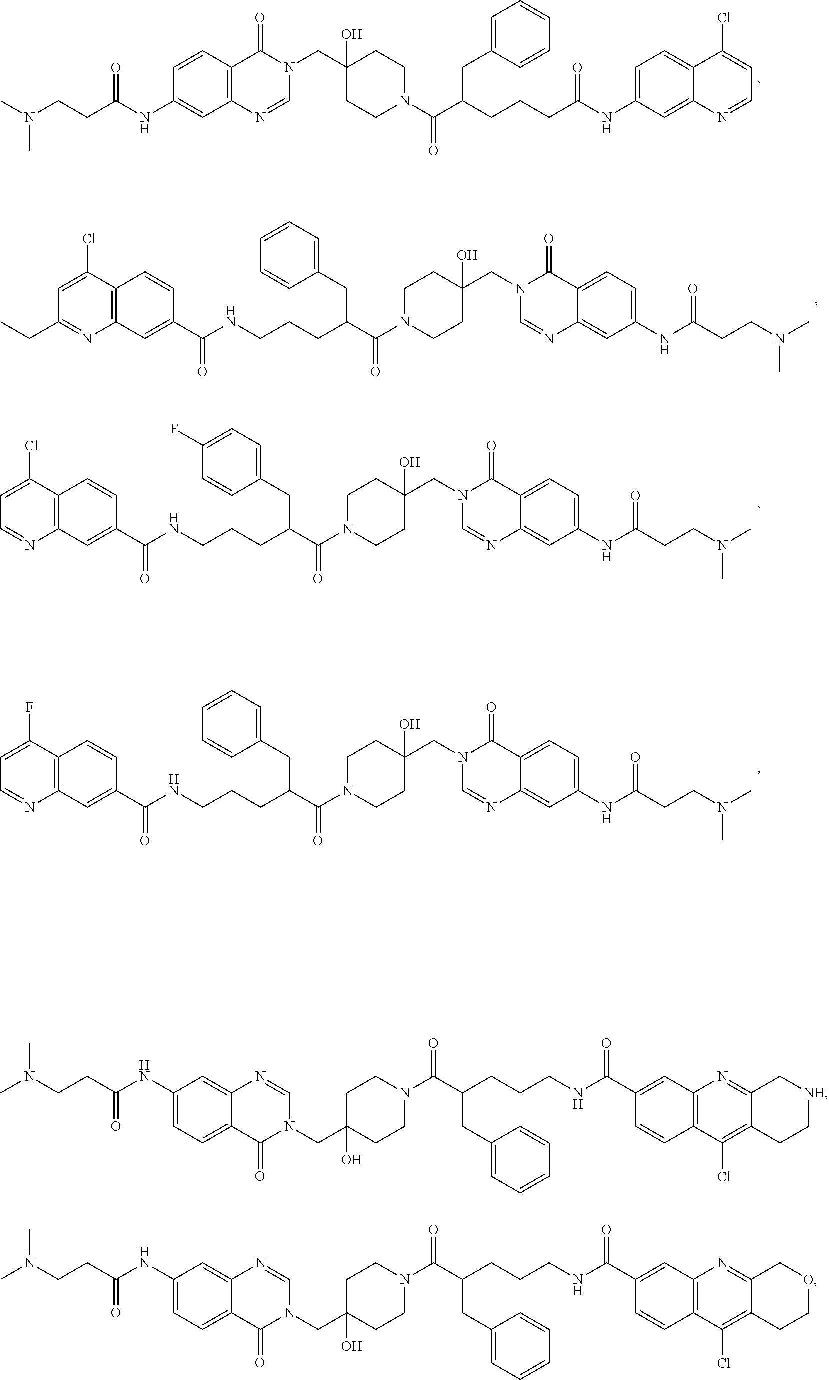 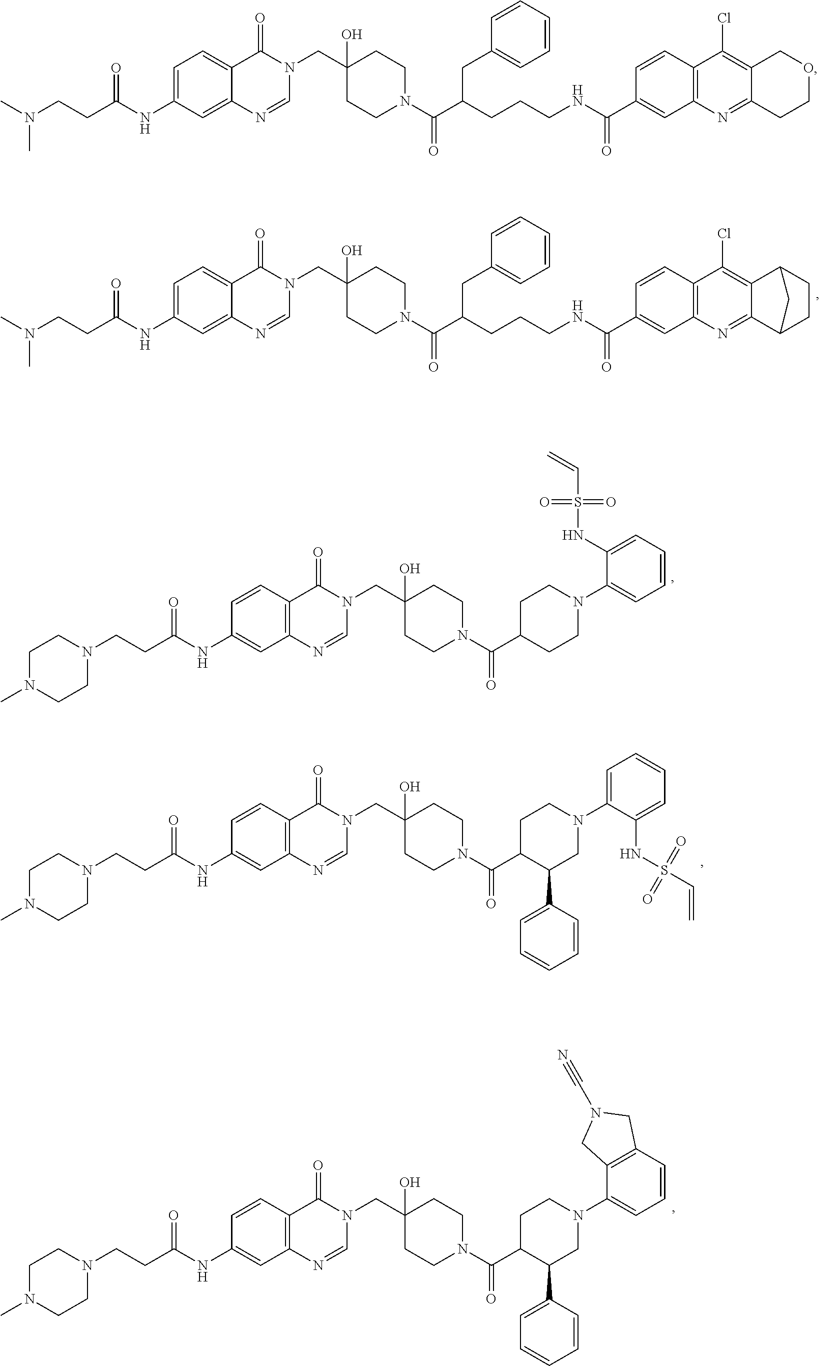 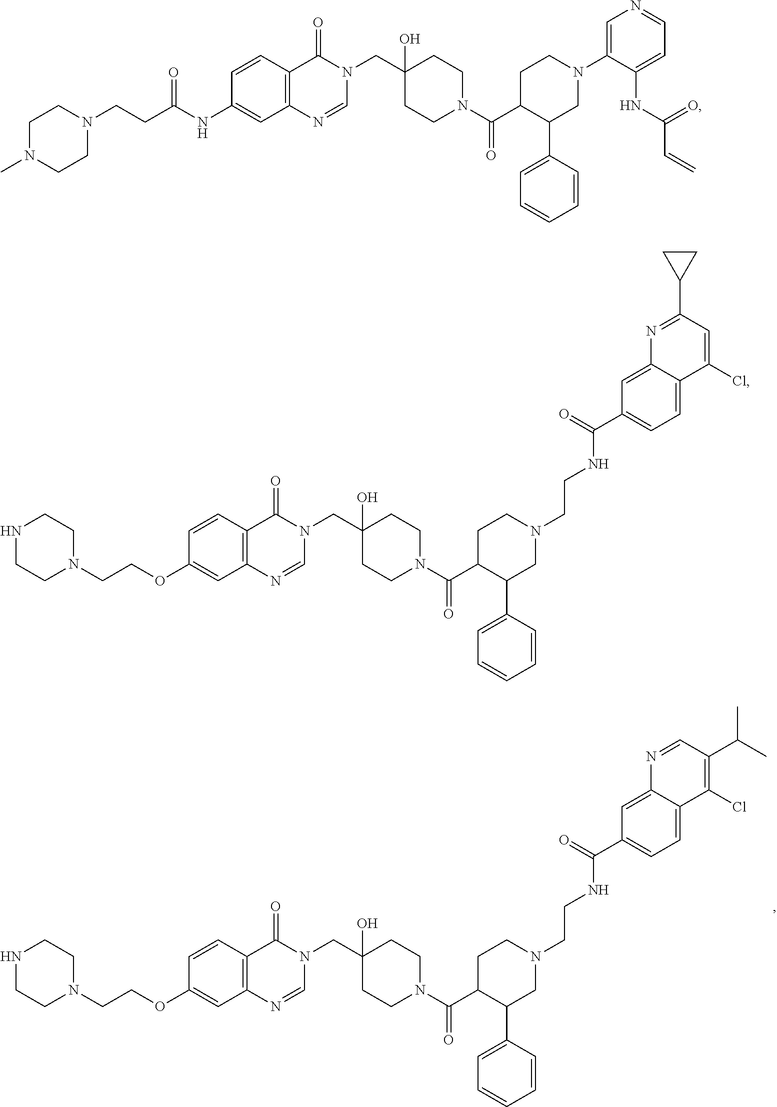 [SMILES: C=CC(=O)Nc1ccncc1N1CCC(C(=O)N2CCC(O)(Cn3cnc4cc(NC(=O)CCN5CCN(C)CC5)ccc4c3=O)CC2)C(c2ccccc2)C1 ; O=C(NCCN1CCC(C(=O)N2CCC(O)(Cn3cnc4cc(OCCN5CCNCC5)ccc4c3=O)CC2)C(c2ccccc2)C1)c1ccc2c(Cl)cc(C3CC3)nc2c1 ; CC(C)c1cnc2cc(C(=O)NCCN3CCC(C(=O)N4CCC(O)(Cn5cnc6cc(OCCN7CCNCC7)ccc6c5=O)CC4)C(c4ccccc4)C3)ccc2c1Cl] 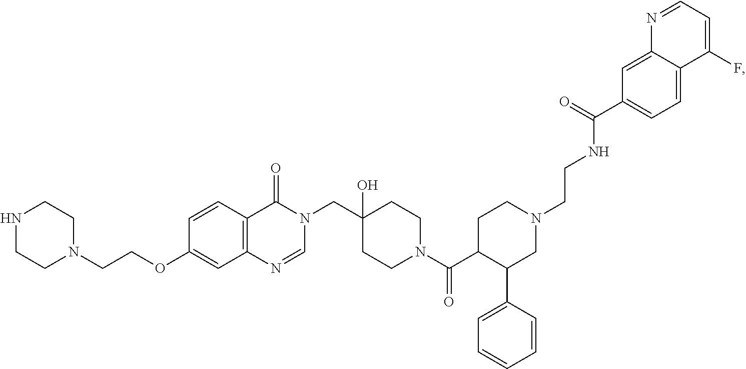 [SMILES: O=C(NCCN1CCC(C(=O)N2CCC(O)(Cn3cnc4cc(OCCN5CCNCC5)ccc4c3=O)CC2)C(c2ccccc2)C1)c1ccc2c(F)ccnc2c1] 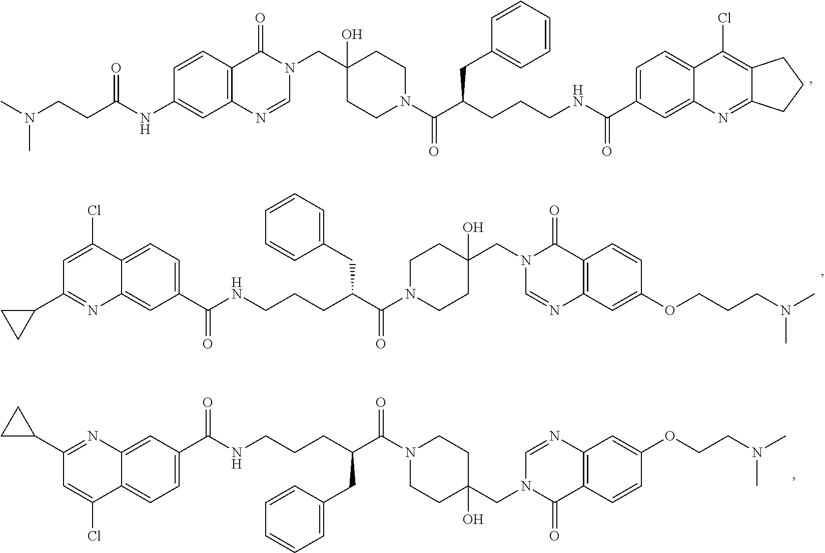 [SMILES: CN(C)CCC(=O)Nc1ccc2c(=O)n(CC3(O)CCN(C(=O)[C@H](CCCNC(=O)c4ccc5c(Cl)c6c(nc5c4)CCC6)Cc4ccccc4)CC3)cnc2c1 ; CN(C)CCCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1 ; CN(C)CCOc1ccc2c(=O)n(CC3(O)CCN(C(=O)[C@H](CCCNC(=O)c4ccc5c(Cl)cc(C6CC6)nc5c4)Cc4ccccc4)CC3)cnc2c1] 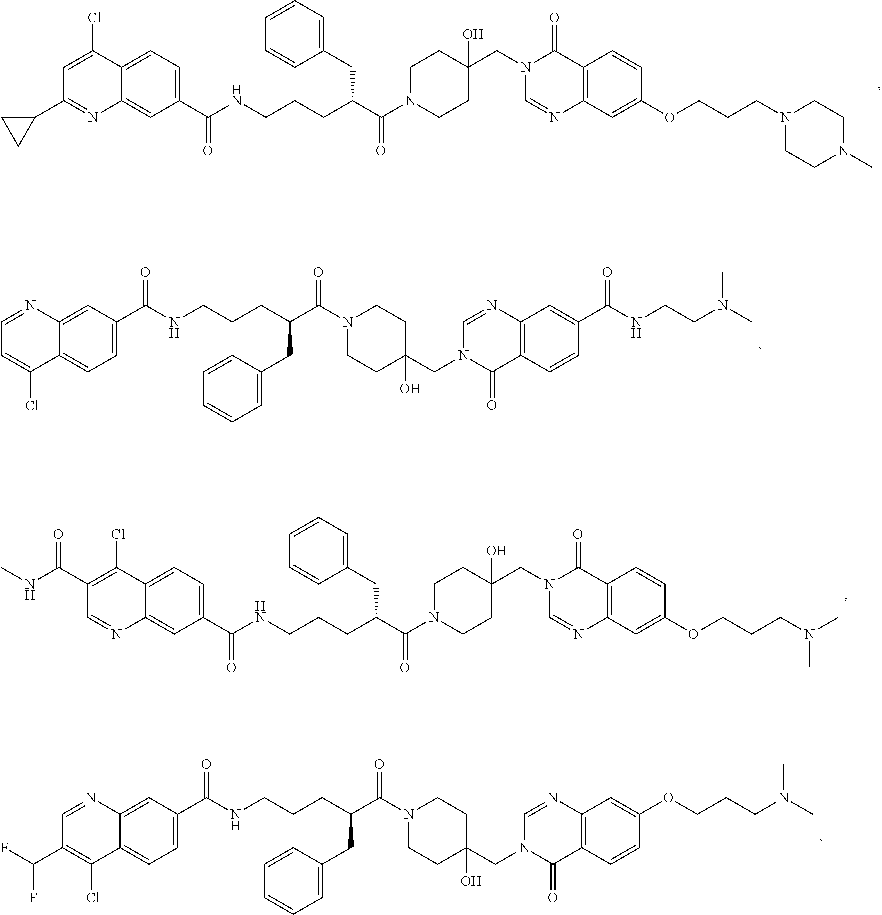 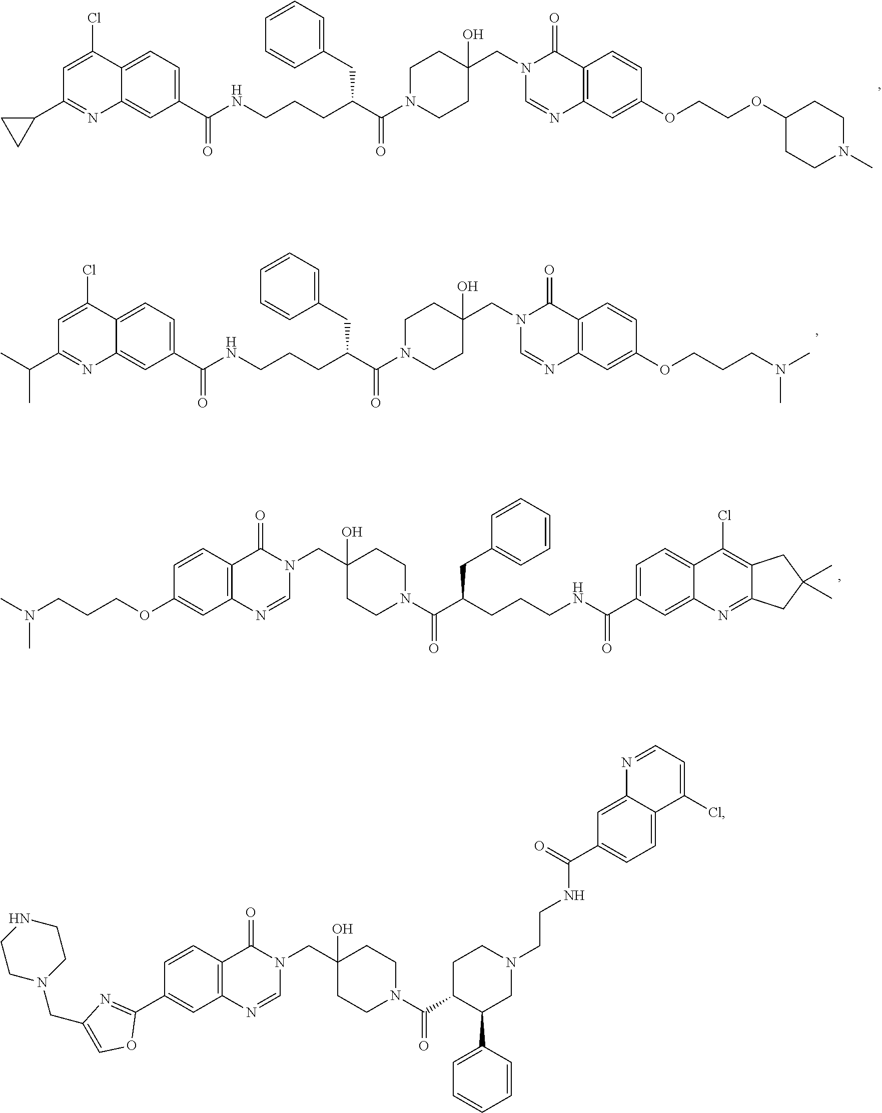 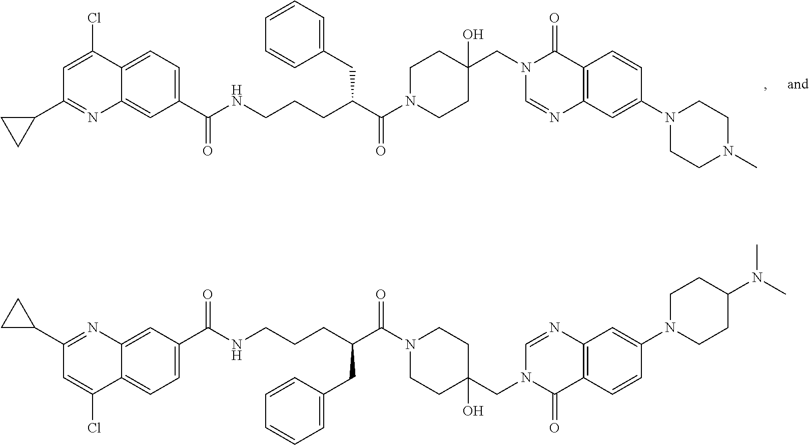 [SMILES: CN1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1 ; CN(C)C1CCN(c2ccc3c(=O)n(CC4(O)CCN(C(=O)[C@H](CCCNC(=O)c5ccc6c(Cl)cc(C7CC7)nc6c5)Cc5ccccc5)CC4)cnc3c2)CC1]
or a pharmaceutically acceptable salt thereof.

76. A pharmaceutical composition comprising a compound of any one of claims 1-75 and a pharmaceutically acceptable carrier.

77. A method of treating a disease or disorder modulated by USP7, comprising administering to a subject in need thereof a compound of any one of claims 1-75 or a pharmaceutical composition of claim 76.

78. A method of inhibiting USP7, comprising administering to a subject in need thereof a compound of any one of claims 1-75 or a pharmaceutical composition of claim 76.

79. The method of claim 77, wherein the disease or disorder associated with inhibition of USP7 is cancer and metastasis, neurodegenerative diseases, immunological disorders, diabetes, bone and joint diseases, osteoporosis, arthritis inflammatory disorders, cardiovascular diseases, ischemic diseases, viral infections and diseases, viral infectivity and/or latency, and bacterial infections and diseases.

80. A method of treating cancer, comprising administering to a subject in need thereof a compound of any one of claims 1-75 or a pharmaceutical composition of claim 76.

81. The method of claim 80, wherein the cancer is liposarcoma, neuroblastoma, glioblastoma, breast cancer, bladder cancer, glioma, adrenocortical cancer, multiple myeloma, colorectal cancer, colon cancer, prostate cancer, non-small cell lung cancer, Human Papilloma Virus-associated cervical cancer, oropharyngeal cancer, penis cancer, ovarian cancer, anal cancer, thyroid cancer, vaginal cancer, Epstein-Barr Virus-associated nasopharyngeal carcinoma, gastric cancer, rectal cancer, thyroid cancer, Hodgkin lymphoma, diffuse large B-cell lymphoma, and Ewing sarcoma.

82. The method of claim 80, wherein the cancer is neuroblastoma, multiple myeloma, breast cancer, glioma, colon cancer, prostate cancer, or ovarian cancer.

83. The method of claim 80, wherein the cancer is multiple myeloma.

84. The method of claim 80, wherein the cancer is Ewing sarcoma.

85. A method of inhibiting USP7, wherein a compound of any one of claims 1-75 forms a covalent bond with USP7.

86. The method of claim 85, wherein the covalent bond forms with a cysteine residue of USP7.

87. Use of a compound of any one of claims 1-75, for the manufacture of a medicament for treating a disease modulated by USP7.

88. A compound of any one of claims 1-75, for use in treating a disease modulated by USP7.
